(19) United States
(12) Patent Application Publication
(10) Pub. No.: US 2022/0102000 A1
(43) Pub. Date: Mar. 31, 2022

(54) PREDICTING BLOOD METABOLITES

(71) Applicant: Yeda Research and Development Co. Ltd., Rehovot (IL)

(72) Inventors: Eran SEGAL, Ramat-HaSharon (IL); Noam BAR, Rehovot (IL); Tal KOREM, Rehovot (IL)

(73) Assignee: Yeda Research and Development Co. Ltd., Rehovot (IL)

(21) Appl. No.: 17/427,223

(22) PCT Filed: Jan. 30, 2020
(86) PCT No.: PCT/IL2020/050121

§ 371 Date: Jul. 30, 2021

(30) Foreign Application Priority Data
Jan. 31, 2019 (IL) 264581

(51) Int. Cl. G16H 50/20; G16B 10/00; G16B 30/10; G16H 20/60; G16B 40/20; C12Q 1/10

(52) U.S. Cl. CPC G16H 50/20; G16B 10/00; C12Q 1/10; G16H 20/60; G16B 40/20; G16B 30/10

(57) ABSTRACT

A method of predicting the quantity of a metabolite in the blood of a subject, accesses a computer readable medium storing a library of trained machine learning procedures, searches the library for a trained machine learning procedure associated with the metabolite, feeds the selected procedure with amount of a plurality of microbes of a microbiome of the subject, and receives from the selected procedure an output indicative of the quantity of the metabolite in the blood.

### RELATED APPLICATION

This application claims the benefit of priority Israeli Patent Application No. 264581 filed Jan. 31, 2019, the contents of which are incorporated herein by reference in their entirety.

### SEQUENCE LISTING STATEMENT

The ASCII file, entitled 80593 Sequence Listing.txt, created on 28 Jan. 2020, comprising 82,571,264 bytes, submitted concurrently with the filing of this application is incorporated herein by reference.

### FIELD AND BACKGROUND OF THE INVENTION

The present invention, in some embodiments thereof, relates to a non-invasive method of quantifying blood metabolites.

Blood serves as a liquid conveyor for molecules inside the body by delivering necessary substances to the cells and transporting metabolic waste products. Of particular importance are the thousands of circulating small molecules termed the serum metabolome, which are either naturally produced by the body or taken up from the environment. While the connection of most of these metabolites to human health is yet to be elucidated, some are known to be predictive diagnostic biomarkers or even causal agents in the development of disease. For example, high blood cholesterol leads to buildup of plaque in the blood vessels, termed atherosclerosis, which in turn increases the risk for a major cardiovascular event such as heart attack, stroke, and peripheral artery disease. As a result, blood cholesterol level serves as both a diagnostic biomarker and a therapeutic target for drugs such as statins. As another example, type II diabetes which impacts around 10% of the population, is diagnosed in part by measurements of blood glucose levels, with a recent study suggesting that a new set of metabolites significantly improves diagnosis. These are only examples for the wealth of potential biomarkers and therapeutic targets that could be found in the blood, making blood an attractive source in which to search for novel biomarkers for early detection and treatment of disease.

Mass spectrometry can accurately identify thousands of metabolites from different biofluids. While some of its identified compounds are well studied and characterized, the determinants of most serum metabolites are still unknown. Studies focusing on human genetics estimated a median heritability of 6.9% for serum metabolites, thereby leaving much of the variation in metabolite levels unaccounted for and suggesting major contributions from environmental factors. Other studies have suggested that the gut microbiome is actively involved in the metabolism of many metabolites which are detectable in human serum, including a diverse set of biochemicals such branched-chain and aromatic amino acids. A notable example is the metabolite trimethylamine N-oxide (TMAO), which is derived from gut microbial metabolism of choline and carnitine, and was reported to act as a marker for cardiovascular disease in humans, with further evidence indicating proatherogenicity and prothromboticity in mouse models. The effect of nutrition on serum metabolites was long established as dietary patterns such as the intake of red meat, whole-grain bread, tea and coffee were linked to changes in a wide range of compounds. Smoking was suggested as impacting serum metabolites, with some of these smoking-related changes in human serum metabolites being reversible after smoking cessation. However, no study to date incorporated all of the above potential determinants within a single human cohort and quantified their relative contribution in explaining serum metabolites.

### SUMMARY OF THE INVENTION

According to an aspect of some embodiments of the present invention there is provided a method of predicting the quantity of a metabolite in the blood of a subject. The method comprises: accessing a computer readable medium storing a library of trained machine learning procedures, each being associated with a different metabolite; searching the library for a trained machine learning procedure associated with the metabolite; feeding the selected procedure with amount of a plurality of microbes of a microbiome of the subject; and receiving from the selected procedure an output indicative of the quantity of the metabolite in the blood.

According to some embodiments of the invention the method comprises measuring the amount of microbes of the microbiome of the subject prior to the analyzing.

According to some embodiments of the invention the microbiome is a fecal microbiome.

According to some embodiments of the invention the plurality of microbes comprises more than 20 microbes.

According to some embodiments of the invention the metabolite is set forth in Table 2.

According to some embodiments of the invention the metabolite is other than glucose and other than cholesterol.

According to some embodiments of the invention at least some of the trained machine learning procedures in the library comprises a set of decision trees.

According to some embodiments of the invention the selected machine learning procedure comprises a set of decision trees, each decision tree comprises a plurality of nodes associated with a respective plurality of decision rules, each decision rule relating to at least one microbe of the microbiome, and wherein a number of decision rules relating to microbes listed in Table 1 is larger than a number of decision rules relating to other microbes of the microbiome.

According to an aspect of some embodiments of the present invention there is provided a method of predicting the quantity of a metabolite set forth in Table 1. The method comprises: accessing a computer readable medium storing a trained machine learning procedure associated with the metabolite; feeding the trained procedure with an amount of N of the corresponding microbes set forth in Table 1, the N being at most 50; and receiving from the procedure an output indicative of the quantity of the metabolite in the blood, thereby predicting the quantity of the metabolite in the blood.

According to some embodiments of the invention the method comprises measuring the amount of microbes of the fecal microbiome of the subject prior to the analyzing.

According to some embodiments of the invention the metabolite is other than glucose and other than cholesterol.

According to an aspect of some embodiments of the present invention there is provided a method of predicting the quantity of a metabolite in the blood of a subject that consumes a diet of a plurality of food types. The method comprises: accessing a computer readable medium storing a library of trained machine learning procedures, each being associated with a different metabolite; searching the library for a trained machine learning procedure associated with the metabolite; feeding the selected procedure with a frequency of consumption of at least 5 of the food types over at least one month and/or a daily mean consumption of at least 5 of the food types; and receiving from the selected procedure an output indicative of the quantity of the metabolite in the blood.

According to some embodiments of the invention the metabolite is other than glucose and other than cholesterol.

According to some embodiments of the invention at least some of the trained machine learning procedures in the library comprises a set of decision trees.

According to some embodiments of the invention each set of decision trees comprises at least 1000 decision trees.

According to some embodiments of the invention the selected machine learning procedure comprises a set of decision trees, each decision tree comprises a plurality of nodes associated with a respective plurality of decision rules, each decision rule relating to at least one food type, and wherein a number of decision rules relating to food types listed in Table 3 is larger than a number of decision rules relating to other food types.

According to an aspect of some embodiments of the present invention there is provided a method of predicting the quantity of a metabolite set forth in Table 3. The method comprises: accessing a computer readable medium storing a trained machine learning procedure associated with the metabolite; feeding the selected procedure with a daily mean consumption and/or frequency of consumption over at least one month of N of the corresponding food types set forth in Table 3 of the subject; and receiving from the selected procedure an output indicative of the quantity of the metabolite in the blood, thereby predicting the quantity of the metabolite in the blood.

According to some embodiments of the invention the N is at most 50.

According to some embodiments of the invention the metabolite is other than glucose and other than cholesterol.

According to some embodiments of the invention the method comprises corroborating the quantity of the metabolite by measuring the amount of the metabolite in a blood sample of the subject.

According to an aspect of some embodiments of the present invention there is provided a method of diagnosing a disease of a subject. The method comprises predicting the quantity of at least one metabolite which is indicative of the disease, wherein the predicting is carried out according to any one of claims 1-21, thereby diagnosing the disease.

According to some embodiments of the invention the disease is selected from the group consisting of a metabolic disease, a cardiovascular disease and kidney disease.

According to an aspect of some embodiments of the present invention there is provided a method of altering the quantity of a metabolite in the blood of the subject. The method comprises: predicting the quantity of the metabolite; and administering to the subject at least one agent which specifically increases or decreases at least one microbe, wherein the agent is selected based on the quantity of the metabolite; wherein the predicting the quantity of the metabolite comprises: accessing a computer readable medium storing a library of trained machine learning procedures, each being associated with a different metabolite; searching the library for a trained machine learning procedure associated with the metabolite; feeding the selected procedure with an amount of a plurality of microbes; and receiving from the selected procedure an output indicative of the quantity of the metabolite in the blood.

According to an aspect of some embodiments of the present invention there is provided a method of altering the amount of a metabolite in the blood of the subject. The method comprises: accessing a computer readable medium storing a library of trained machine learning procedures, each being associated with a different metabolite; searching the library for a trained machine learning procedure associated with the metabolite; feeding the selected procedure with a predetermined quantity of the metabolite; receiving from the selected procedure an output indicative of at least one microbe; and administering to the subject at least one agent which specifically increases or decreases the amount of the at least one microbe, thereby altering the amount of the metabolite in the blood of the subject.

According to some embodiments of the invention the agent which increases the microbe is a probiotic.

According to some embodiments of the invention the agent which decreases the microbe is an antibiotic or a phage directed to the microbe.

According to an aspect of some embodiments of the present invention there is provided a method of providing dietary advice to a subject. The method comprises predicting the quantity of a metabolite in the blood by carrying out the method according to claim 14-22, wherein when the metabolite is above or below the recommended quantity of the metabolite, recommending consumption of at least one food type that alters the quantity of the metabolite.

According to some embodiments of the invention the metabolite is set forth in Table 4.

According to some embodiments of the invention the food type is the corresponding food type set forth in Table 4.

According to an aspect of some embodiments of the present invention there is provided a method of altering the amount of a metabolite set forth in Table 3 in the blood of the subject. The method comprises: accessing a computer readable medium storing a library of trained machine learning procedures, each being associated with a different metabolite; searching the library for a trained machine learning procedure associated with the metabolite; feeding the selected procedure with a predetermined quantity of the metabolite; receiving from the selected procedure an output indicative of a list of food types; and providing dietary advice to the subject, based on the output.

According to some embodiments of the invention the method comprises predicting the amount of the metabolite using another trained machine learning procedure.

Unless otherwise defined, all technical and/or scientific terms used herein have the same meaning as commonly understood by one of ordinary skill in the art to which the invention pertains. Although methods and materials similar or equivalent to those described herein can be used in the practice or testing of embodiments of the invention, exemplary methods and/or materials are described below. In case of conflict, the patent specification, including definitions, will control. In addition, the materials, methods, and examples are illustrative only and are not intended to be necessarily limiting.

### BRIEF DESCRIPTION OF THE DRAWINGS

Some embodiments of the invention are herein described, by way of example only, with reference to the accompanying drawings. With specific reference now to the drawings in detail, it is stressed that the particulars shown are by way of example and for purposes of illustrative discussion of embodiments of the invention. In this regard, the description taken with the drawings makes apparent to those skilled in the art how embodiments of the invention may be practiced.

In the drawings:

FIGS. 1A-E. Accurate and reproducible serum metabolomics from a deeply phenotyped human cohort. (A) Illustration of the measurements we obtained from our cohort. (B) Basic characteristics and demographics of our main and replication cohorts. P-values were calculated using Mann-Whitney U test for continuous variables and Fisher's exact test for binary variables. (C) Breakdown of the 1251 measured metabolites by type. (D) Number of samples (y-axis) in which each metabolite (x-axis) was identified, sorted by prevalence. (E) Spearman correlations (y-axis; box—IQR, whiskers—IQR*1.5) between standardized metabolomic profiles (Methods) of different individuals (n=475; median Spearman 0.05, std=0.12) stratified by sex, and between standardized metabolomic profiles of the same participant (n=20; median Spearman 0.68, std=0.06) taken one week apart. C&V, Cofactors and vitamins; std, Standard deviation.

FIGS. 2A-F. Diet, gut microbiome, genetics and clinical data predict the levels of most serum metabolites. Figure panels refer to results of 5-fold cross validation predictions of the levels of every metabolite based on models derived separately for each feature group. An exception is human genetics for which the EV of each metabolite is determined as that of the single most associated SNP. (A) Box and swarm plots (box, IQR; whiskers, 1.5*IQR) showing the EV (R²) of the top 50 predicted metabolites of each feature group (group names below panel C). Feature groups are sorted by their median EV across these 50 metabolites. (B) Heatmap showing the 95% confidence interval (CI) for EV (color gradient from left to right corresponds to lower and higher CI bounds) predicted for each metabolite (y-axis) by every feature group (x-axis). Only metabolites with significant predictions after strict Bonferonni correction are shown, their number per column shown above panel B. P-values and CIs were estimated using bootstrapping (Methods). (C) Enrichment of metabolite types in the metabolites predicted by each feature group (Mann-Whitney U test; Methods). Only significant enrichments are shown (p<0.05 after 10% FDR correction). Exact p-values are written in each cell. (D) A histogram of the number of metabolites (y-axis) with any value of EV (x-axis) as obtained using the full model. Inset shows the metabolites with EV in the range of 0.3-0.8. (E) Spearman correlations computed between the EV of metabolites for every pair of feature groups. Rows and columns are hierarchically clustered using Euclidean distances between the Spearman correlations. (F) The fraction of total EV (x-axis) of each feature group (y-axis) compared to the total EV of a model with all feature groups excluding genetics (full model). Total EV is the sum of the EV of the first 15 metabolite principal components (PCs) weighted by the EV of each PC (Methods).

FIGS. 3A-C. Validation of metabolite predictions on an independent cohort. (A) R² multiplied by the sign of the Pearson correlation coefficient (x-axis) between metabolite levels and BMI in our study, versus the mean R² multiplied by the sign of the Pearson correlation coefficient (y-axis) of BMI associated metabolites recently reported by a different group. Shown are 36 (out of 49) BMI associated metabolites that were also measured in this cohort. Line and shaded coloring represent the fitting of a linear model and the 95% confidence interval. (B-C) Dot plots showing the R² of metabolites obtained from prediction models trained on the main cohort (x-axis) and evaluated on the validation cohort (y-axis), for models based on microbiome (B) and diet (C) features. Only metabolites for which we obtained statistically significant predictions with over 5% of their variance explained in the main cohort are presented.

FIGS. 4A-F. Diet and gut microbiome data independently explain a wide range of biochemicals. (A) Shown is the EV of every metabolite from prediction models based on the gut microbiome (x-axis) versus diet (y-axis). Dashed red line is y=x. (B) Same for prediction models based on both gut microbiome and diet (x-axis) compared to using only diet (y-axis). (C) A histogram of the differences between the axes in B for metabolites whose predictions were statistically significant and over 5% of their variance was explained in at least one of the models. (D) Shown is the EV of every metabolite from prediction models based on all gut microbiome features (x-axis) compared to using only the top predictor of that metabolite, selected as the feature with the largest mean absolute SHAP value (y-axis). Dashed red palette lines mark different y:x ratios. (E) The levels of the unknown compound X-16124 in individuals for which the bacterial taxa from the Eggerthellaceae family was detectable in stool versus individuals for which it was not. *** Mann-Whitney U p<0.001; (F) Heatmap showing the directional mean absolute SHAP values (Methods) of various features (x-axis) computed from 5-fold cross validation models that predict metabolite levels (y-axis) using two separate models, one based on diet and another on gut microbiome data. Positive SHAP values indicate that higher feature values lead, on average, to higher predicted values, while negative SHAP values indicate that lower feature values lead, on average, to lower predicted values. Metabolites are sorted by their type and clustered within each group. Shown are the top 200 predicted metabolites using diet and gut microbiome, and the top 50 features by maximum mean absolute SHAP value across all metabolites. C&V, Cofactors and vitamins; AAs, Amino Acids.

FIGS. 5A-D. Networks of interactions between phenotypes explain diverse metabolites. Interactions between features from different feature groups predictive of similar metabolites are presented in a graphical layout, in which nodes are either metabolites or features, and edges are the directional mean absolute SHAP values (Methods) computed from models trained only on features from the respective feature group. Circular nodes—metabolites; predictive feature nodes—squares; both colored by relevant categories. Shown are only edges with a mean absolute SHAP value greater than 0.12. (A) Network of associations for the following feature groups: macronutrients, diet, microbiome, lifestyle, drugs and seasonal effects. (B) A large group of metabolites which their predictions are mainly driven by the reported consumption of coffee and the relative abundance of a bacteria from the Clostridiales order. (C) Metabolites explained by seasonal fruit consumption. (D) Selected examples of interactions between metabolites and features in predictive models.

FIGS. 6A-F. Metabolites explained by bread increase following an intervention that increases bread consumption. (A) Measuring associations between dietary features and metabolite levels using samples from this study. (B) Histogram of directional mean absolute SHAP values of whole-wheat bread consumption for metabolites computed based on held-out samples from our cohort. The top 5% (n=62; blue) positively associated metabolites and the top 5% (n=62; red) negatively associated metabolites are marked and used for further analysis. (C) A randomized controlled trial with 20 healthy subjects comparing the effect of consuming traditionally milled and prepared whole-grain sourdough bread to that of consuming industrial white bread made from refined wheat. We analyzed samples from the first week of the trial, in which 10 subjects increased consumption of sourdough bread and 10 others increased consumption of white bread. (D) Box plots (box, IQR; whiskers, 1.5*IQR) showing the mean fold-change (FC) of the top 5% positively (blue) and negatively (red) associated metabolites, separated by intervention group. Among the group which received the sourdough bread intervention the mean FC of the top 5% positively associated metabolites was significantly higher than the mean FC of the top 5% negatively associated metabolites (p<10⁻¹², Mann-Whitney U). *** Mann-Whitney U p<0.001; n.s., Not significant. (E-F) FC (y-axis) of two metabolites separated by intervention groups. In the sourdough bread group the FC of both betaine (E; Mann-Whitney U p<0.004) and cytosine (F; Mann-Whitney U p<0.002) were higher compared to the same FC in the group having white bread.

FIGS. 7A and 7B show results of experiments in which the model of the present embodiments was applied, without modification, to an independent cohort demonstrating a cross-cohort prediction ability.

FIGS. 8A and 8B. Validating metabolomics accuracy by comparing measurements to standard lab tests. Mass-spectrometry measurements (y-axis) versus standardized lab tests results (x-axis; Methods) for creatinine (E; Pearson R=0.87, p<10-20) and cholesterol (F; R=0.79, p<10-20). a.u., Arbitrary units.

FIGS. 9A-E. Gradient boosting decision trees outperform Lasso regression on diet and microbiome data. (A) Metabolite prediction R2 of GBDT vs Lasso regression models using diet data. Shown are only metabolites for which both models achieved significant predictions with R2 above 0.05. (B) Histogram of the differences between the R2 of GBDT compared to Lasso regression using the diet data. (C) The levels of the metabolite hydroxy-CMPF* vs the monthly consumption of cooked, baked or grilled fish as reported in a food frequency questionnaire. The comparison of Spearman and Pearson correlation coefficients suggests that the relationship between the metabolite and the numerical values of the question are monotonic yet non-linear, which explains why GBDT performs better in predicting the levels of hydroxy-CMPF* from diet data. The x-axis is not in scale. (D-E) Same as A-B for microbiome. GBDT, Gradient Boosting Decision Trees; a.u., arbitrary units.

FIG. 10. Comparison of explained variance of metabolites for every pair of feature groups. Every panel shows a dot plot of the explained variance of the metabolite groups from models based on every pair of feature groups. Panels on the diagonal shows the marginal distribution of explained variance of metabolite groups for a certain feature group.

FIG. 11 is a schematic illustration of a computer readable medium storing a library of trained machine learning (ML) procedures, according to some embodiments of the present invention.

FIG. 12 is a schematic illustration of a method suitable for predicting a quantity of a metabolite using a machine learning procedure which is associated with the metabolite and which is trained using microbiome data, according to some embodiments of the present invention.

FIG. 13 is a schematic illustration of a method suitable for predicting a quantity of a metabolite using a machine learning procedure which is associated with the metabolite and which is trained using food consumption data, according to some embodiments of the present invention.

FIG. 14 is a schematic illustration of a method suitable for solving an inverse problem using a machine learning procedure which is trained using microbiome data, according to some embodiments of the present invention.

FIG. 15 is a schematic illustration of a method suitable for solving an inverse problem using a machine learning procedure which is trained using food consumption data, according to some embodiments of the present invention.

FIG. 16. Principal component analysis over the metabolomics data. Shown are the proportion of variance explained by each of the first 400 principal components (left y-axis; black) and their cumulative EV (right y-axis; blue).

FIG. 17. Overall predictive power of gut microbiome and diet data replicates in an independent cohort. The sum of the explained variance (y-axis, R2) for diet and microbiome (x-axis) in the main (blue) and replication (red) cohorts. Shown are only metabolites for which the models achieved significant out-of-sample predictions with R² above 0.05 in the main cohort.

FIG. 18. Replication of associations between genetic loci and the levels of circulating blood metabolites. Explained variance (R²) of a model based on top significantly associated SNPs in the TwinsUK cohort from a previous study6 (x-axis) vs the explained variance of a model based on a single top associated SNP from this study (y-axis). Shown are results for 301 metabolites which were measured in both studies. Line and shaded coloring represent the fitting of a linear model and the 95% confidence interval.

FIGS. 19A-F. Specific dietary features and bacterial taxa underlie the accurate prediction of circulating metabolites. (A-F) Predicted (y-axis) vs measured (x-axis) levels (arbitrary units) of X-16124 (A; Pearson R=0.77, p<10-20), phenylacetylglutamine (B; R=0.63, p<10-20), p-cresol-glucuronide (C; R=0.64, p<10-20), caffeine (D; R=0.68, p<10-20), hydroxy-CMPF (E; R=0.72, p<10-20) and stachydrine (F; R=0.5, p<10-20). Predictions of A-C are based only on microbiome data, and colored by the relative abundance of the bacterial taxa having the highest mean absolute SHAP value for each metabolite. Predictions of D-F are based only on diet data, and colored by the reported consumption of the dietary item having the highest mean absolute SHAP value for each metabolite. p-values for prediction were estimated via bootstrapping.

FIG. 20. Distribution of bacterial phyla in our cohort. Stacked bar plots per sample (x-axis) showing the relative abundance of bacterial phyla (y-axis). Samples are sorted by the relative abundance of the most abundant phylum, Firmicutes. Bacteroidetes is the second most abundant phylum in our cohort. Relative abundance of a phylum is computed as the sum over relative abundances of all bacterial features belonging to that phylum.

### DESCRIPTION OF SPECIFIC EMBODIMENTS OF THE INVENTION

The present invention, in some embodiments thereof, relates to a non-invasive method of quantifying blood metabolites.

Before explaining at least one embodiment of the invention in detail, it is to be understood that the invention is not necessarily limited in its application to the details set forth in the following description or exemplified by the Examples. The invention is capable of other embodiments or of being practiced or carried out in various ways.

The collection of metabolites circulating in the human blood, termed the serum metabolome, contains a plethora of biomarkers and causative agents. Although the origin of specific compounds is known, the understanding of the key determinants of most metabolites is poor.

The present inventors have now measured the levels of 1251 circulating metabolites in 521 serum samples from a healthy cohort, and devised machine learning algorithms to predict their levels in held-out subjects based on a comprehensive profile consisting of gut microbiome, clinical parameters, diet, lifestyle, anthropometric measurements and medication data. Notably, they obtained significant predictions for over 92% of the profiled metabolites, with diet and microbiome each explaining hundreds of metabolites, and with 64% of the variance of some metabolites explained using only gut microbiome data. To corroborate the causality of these predictions, the present inventors showed that some metabolites that were predicted to be positively associated with bread increased in levels following a randomized clinical trial of bread intervention. Overall, the present results unravel the potential determinants of over 1000 metabolites, paving the way towards mechanistic understanding of the alterations in metabolites under different conditions and to designing interventions for manipulating metabolite levels.

Thus, according to a first aspect of the present invention there is provided a method of predicting the quantity of a metabolite in the blood of a subject, the method comprising analyzing the amount of a plurality of microbes of a microbiome of the subject so as to reach a confidence level of at least 95% in the significance of the predictions, thereby predicting the quantity of the metabolite in the blood.

The methods described herein are preferably non-invasive methods. Thus, in one embodiment, the methods described herein are carried out without blood sampling.

As used herein the term “subject” refers to a mammalian subject (e.g. mouse, cow, dog, cat, horse, monkey, human), preferably human.

In one embodiment, the subject is a healthy subject.

As used herein, a “metabolite” is an intermediate or product of metabolism. The term metabolite is generally restricted to small molecules and does not include polymeric compounds such as DNA or proteins greater than 100 amino acids in length. A metabolite may serve as a substrate for an enzyme of a metabolic pathway, an intermediate of such a pathway or the product obtained by the metabolic pathway.

In preferred embodiments, metabolites include but are not limited to sugars, organic acids, amino acids, fatty acids, hormones, vitamins, as well as ionic fragments thereof. In another embodiment, the metabolite is an oligopeptides (less than about 100 amino acids in length). In still another embodiment, the metabolite is not a peptide or a nucleic acid.

In particular, the metabolites are less than about 3000 Daltons in molecular weight, and more particularly from about 50 to about 3000 Daltons.

The metabolite of this aspect of the present invention may be a primary metabolite (i.e. essential to the microbe for growth) or a secondary metabolite (one that does not play a role in growth, development or reproduction, and is formed during the end or near the stationary phase of growth.

Representative examples of metabolic pathways in which the metabolites of the present invention are involved include, without limitation, citric acid cycle, respiratory chain, photosynthesis, photorespiration, glycolysis, gluconeogenesis, hexose monophosphate pathway, oxidative pentose phosphate pathway, production and β-oxidation of fatty acids, urea cycle, amino acid biosynthesis pathways, protein degradation pathways such as proteasomal degradation, amino acid degrading pathways, biosynthesis or degradation of: lipids, polyketides (including, e.g., flavonoids and isoflavonoids), isoprenoids (including, e.g., terpenes, sterols, steroids, carotenoids, xanthophylls), carbohydrates, phenylpropanoids and derivatives, alkaloids, benzenoids, indoles, indole-sulfur compounds, porphyrines, anthocyans, hormones, vitamins, cofactors such as prosthetic groups or electron carriers, lignin, glucosinolates, purines, pyrimidines, nucleosides, nucleotides and related molecules such as tRNAs, microRNAs (miRNA) or mRNAs.

Preferably, the metabolite is set forth in the Human Metabolite Database which is available online at wwwdothmdb.ca/metabolites.

Exemplary metabolites that may be analyzed include, but are not limited to: (N(1)+N(8))-acetylspermidine, “1,2,3-benzenetriol sulfate (1)”, “1,2,3-benzenetriol sulfate (2)”, “1,2-dilinoleoyl-GPC (18:2/18:2)”, “1,2-dilinoleoyl-GPE (18:2/18:2)*”, “1,2-dipalmitoyl-GPC (16:0/16:0)”, “1,3,7-trimethylurate”, “1,3-dimethylurate”, “1,5-anhydroglucitol (1,5-AG)”, “1,7-dimethylurate”, 1-(1-enyl-oleoyl)-GPE (P-18:1)*, 1-(1-enyl-palmitoyl)-2-arachidonoyl-GPC (P-16:0/20:4)*, 1-(1-enyl-palmitoyl)-2-arachidonoyl-GPE (P-16:0/20:4)*, 1-(1-enyl-palmitoyl)-2-linoleoyl-GPC (P-16:0/18:2)*, 1-(1-enyl-palmitoyl)-2-linoleoyl-GPE (P-16:0/18:2)*, 1-(1-enyl-palmitoyl)-2-oleoyl-GPC (P-16:0/18:1)*, 1-(1-enyl-palmitoyl)-2-oleoyl-GPE (P-16:0/18:1)*, 1-(1-enyl-palmitoyl)-2-palmitoleoyl-GPC (P-16:0/16:1)*, 1-(1-enyl-palmitoyl)-2-palmitoyl-GPC (P-16:0/16:0)*, 1-(1-enyl-palmitoyl)-GPC (P-16:0)*, 1-(1-enyl-palmitoyl)-GPE (P-16:0)*, 1-(1-enyl-stearoyl)-2-arachidonoyl-GPE (P-18:0/20:4)*, 1-(1-enyl-stearoyl)-2-linoleoyl-GPE (P-18:0/18:2)*, 1-(1-enyl-stearoyl)-2-oleoyl-GPE (P-18:0/18:1), 1-(1-enyl-stearoyl)-GPE (P-18:0)*, 1-arachidonoyl-GPA (20:4), 1-arachidonoyl-GPC (20:4n6)*, 1-arachidonoyl-GPE (20:4n6)*, 1-arachidonoyl-GPI (20:4)*, 1-arachidonylglycerol (20:4), 1-dihomo-linolenylglycerol (20:3), 1-dihomo-linoleoylglycerol (20:2), 1-docosahexaenoylglycerol (22:6), 1-lignoceroyl-GPC (24:0), 1-linolenoyl-GPC (18:3)*, 1-linolenoylglycerol (18:3), 1-linoleoyl-2-arachidonoyl-GPC (18:2/20:4n6)*, 1-linoleoyl-2-linolenoyl-GPC (18:2/18:3)*, 1-linoleoyl-GPA (18:2)*, 1-linoleoyl-GPC (18:2), 1-linoleoyl-GPE (18:2)*, 1-linoleoyl-GPG (18:2)*, 1-linoleoyl-GPI (18:2)*, 1-linoleoylglycerol (18:2), 1-methylhistidine, 1-methylimidazoleacetate, 1-methylnicotinamide, 1-methylurate, 1-methylxanthine, 1-myristoyl-2-arachidonoyl-GPC (14:0/20:4)*, 1-myristoyl-2-palmitoyl-GPC (14:0/16:0), 1-myristoylglycerol (14:0), 1-oleoyl-2-docosahexaenoyl-GPC (18:1/22:6)*, 1-oleoyl-2-docosahexaenoyl-GPE (18:1/22:6)*, 1-oleoyl-GPC (18:1), 1-oleoyl-GPE (18:1), 1-oleoyl-GPG (18:1)*, 1-oleoyl-GPI (18:1)*, 1-oleoylglycerol (18:1), 1-palmitoleoyl-2-linolenoyl-GPC (16:1/18:3)*, 1-palmitoleoyl-2-linoleoyl-GPC (16:1/18:2)*, 1-palmitoleoyl-GPC (16:1)*, 1-palmitoleoylglycerol (16:1)*, 1-palmitoyl-2-arachidonoyl-GPC (16:0/20:4n6), 1-palmitoyl-2-arachidonoyl-GPE (16:0/20:4)*, 1-palmitoyl-2-arachidonoyl-GPI (16:0/20:4)*, 1-palmitoyl-2-docosahexaenoyl-GPC (16:0/22:6), 1-palmitoyl-2-docosahexaenoyl-GPE (16:0/22:6)*, 1-palmitoyl-2-gamma-linolenoyl-GPC (16:0/18:3n6)*, 1-palmitoyl-2-linoleoyl-GPC (16:0/18:2), 1-palmitoyl-2-linoleoyl-GPE (16:0/18:2), 1-palmitoyl-2-linoleoyl-GPI (16:0/18:2), 1-palmitoyl-2-oleoyl-GPC (16:0/18:1), 1-palmitoyl-2-oleoyl-GPE (16:0/18:1), 1-palmitoyl-2-oleoyl-GPI (16:0/18:1)*, 1-palmitoyl-2-palmitoleoyl-GPC (16:0/16:1)*, 1-palmitoyl-GPA (16:0), 1-palmitoyl-GPC (16:0), 1-palmitoyl-GPE (16:0), 1-palmitoyl-GPG (16:0)*, 1-palmitoyl-GPI (16:0), 1-palmitoylglycerol (16:0), 1-stearoyl-2-arachidonoyl-GPC (18:0/20:4), 1-stearoyl-2-arachidonoyl-GPE (18:0/20:4), 1-stearoyl-2-arachidonoyl-GPI (18:0/20:4), 1-stearoyl-2-docosahexaenoyl-GPC (18:0/22:6), 1-stearoyl-2-docosahexaenoyl-GPE (18:0/22:6)*, 1-stearoyl-2-linoleoyl-GPC (18:0/18:2)*, 1-stearoyl-2-linoleoyl-GPE (18:0/18:2)*, 1-stearoyl-2-linoleoyl-GPI (18:0/18:2), 1-stearoyl-2-oleoyl-GPC (18:0/18:1), 1-stearoyl-2-oleoyl-GPE (18:0/18:1), 1-stearoyl-2-oleoyl-GPI (18:0/18:1)*, 1-stearoyl-2-oleoyl-GPS (18:0/18:1), 1-stearoyl-GPC (18:0), 1-stearoyl-GPE (18:0), 1-stearoyl-GPG (18:0), 1-stearoyl-GPI (18:0), 1-stearoyl-GPS (18:0)*, 10-heptadecenoate (17:1n7), 10-nonadecenoate (19:1n9), 10-undecenoate (11:1n1), “12,13-DiHOME”, 12-HETE, 12-HHTrE, 13-HODE+9-HODE, 13-methylmyristate, 14-HDoHE/17-HDoHE, 15-methylpalmitate, 16a-hydroxy DHEA 3-sulfate, 17-methylstearate, 17alpha-hydroxypregnanolone glucuronide, 17alpha-hydroxypregnenolone 3-sulfate, 1H-indole-7-acetic acid, 2′-deoxyuridine, 2′-O-methylcytidine, 2′-O-methyluridine, “2,3-dihydroxy-2-methylbutyrate”, “2,3-dihydroxyisovalerate”, “2,3-dihydroxypyridine”, 2-acetamidophenol sulfate, 2-aminoadipate, 2-aminobutyrate, 2-aminoheptanoate, 2-aminooctanoate, 2-aminophenol sulfate, 2-arachidonoylglycerol (20:4), 2-docosahexaenoylglycerol (22:6)*, 2-hydroxy-3-methylvalerate, 2-hydroxyacetaminophen sulfate*, 2-hydroxyadipate, 2-hydroxybehenate, 2-hydroxybutyrate/2-hydroxyisobutyrate, 2-hydroxydecanoate, 2-hydroxyglutarate, 2-hydroxyhippurate (salicylurate), 2-hydroxyibuprofen, 2-hydroxylaurate, 2-hydroxynervonate*, 2-hydroxyoctanoate, 2-hydroxypalmitate, 2-hydroxyphenylacetate, 2-hydroxystearate, 2-keto-3-deoxy-gluconate, 2-linoleoylglycerol (18:2), 2-methoxyacetaminophen glucuronide*, 2-methoxyacetaminophen sulfate*, 2-methoxyresorcinol sulfate, 2-methylbutyrylcarnitine (C5), 2-methylcitrate/homocitrate, 2-methylserine, 2-oleoylglycerol (18:1), 2-oxoarginine*, 2-palmitoleoyl-GPC (16:1)*, 2-palmitoyl-GPC (16:0)*, 2-palmitoylglycerol (16:0), 2-piperidinone, 2-pyrrolidinone, 2-stearoyl-GPE (18:0)*, 21-hydroxypregnenolone disulfate, “3,4-methyleneheptanoate”, “3,7-dimethylurate”, 3-(3-hydroxyphenyl)propionate, 3-(3-hydroxyphenyl)propionate sulfate, 3-(4-hydroxyphenyl)lactate, 3-(cystein-S-yl)acetaminophen*, 3-(N-acetyl-L-cystein-S-yl) acetaminophen, 3-acetylphenol sulfate, 3-aminoisobutyrate, 3-carboxy-4-methyl-5-propyl-2-furanpropanoate (CMPF), 3-hydroxy-2-ethylpropionate, 3-hydroxy-3-methylglutarate, 3-hydroxybutyrate (BHBA), 3-hydroxybutyrylcarnitine (1),3-hydroxybutyrylcarnitine (2),3-hydroxycotinine glucuronide, 3-hydroxydecanoate, 3-hydroxyhexanoate, 3-hydroxyhippurate, 3-hydroxyisobutyrate, 3-hydroxylaurate, 3-hydroxyoctanoate, 3-hydroxypyridine sulfate, 3-hydroxyquinine, 3-indoxyl sulfate, 3-methoxycatechol sulfate (1),3-methoxycatechol sulfate (2),3-methoxytyramine sulfate, 3-methoxytyrosine, 3-methyl catechol sulfate (1),3-methyl catechol sulfate (2), 3-methyl-2-oxobutyrate, 3-methyl-2-oxovalerate, 3-methyladipate, 3-methylcytidine, 3-methylglutaconate, 3-methylglutarylcarnitine (2),3-methylhistidine, 3-methylxanthine, 3-phenylpropionate (hydrocinnamate), 3-sulfo-L-alanine, 3-ureidopropionate, 3b-hydroxy-5-cholenoic acid, 3beta-hydroxy-5-cholestenoate, 4-acetamidobenzoate, 4-acetamidobutanoate, 4-acetamidophenol, 4-acetamidophenylglucuronide, 4-acetaminophen sulfate, 4-acetylphenol sulfate, 4-allylphenol sulfate, 4-ethylphenylsulfate, 4-guanidinobutanoate, 4-hydroxybenzoate, 4-hydroxychlorothalonil, 4-hydroxycinnamate sulfate, 4-hydroxycoumarin, 4-hydroxyhippurate, 4-hydroxyphenylacetate, 4-hydroxyphenylpyruvate, 4-imidazoleacetate, 4-methyl-2-oxopentanoate, 4-methylcatechol sulfate, 4-vinylguaiacol sulfate, 4-vinylphenol sulfate, “5,6-dihydrothymine”, 5-(galactosylhydroxy)-L-lysine, 5-acetylamino-6-amino-3-methyluracil, 5-acetylamino-6-formylamino-3-methyluracil, 5-bromotryptophan, 5-dodecenoate (12:1n7), 5-hydroxyhexanoate, 5-hydroxyindoleacetate, 5-hydroxylysine, 5-hydroxymethyl-2-furoic acid, 5-methylthioadenosine (MTA), 5-methyluridine (ribothymidine), 5-oxoproline, “5alpha-androstan-3alpha,17alpha-diol monosulfate”, “5 alpha-androstan-3 alpha,17beta-diol disulfate”, “5alpha-androstan-3alpha,17beta-diol monosulfate (1)”, “5 alpha-androstan-3alpha,17beta-diol monosulfate (2)”, “5alpha-androstan-3beta,17alpha-diol disulfate”, “5alpha-androstan-3beta,17beta-diol disulfate”, “5alpha-androstan-3beta,17beta-diol monosulfate (2)”, “5alpha-pregnan-3 (alpha or beta),20beta-diol disulfate”, “5alpha-pregnan-3beta,20alpha-diol disulfate”, “5alpha-pregnan-3beta,20alpha-diol monosulfate (1)”, “5alpha-pregnan-3beta,20alpha-diol monosulfate (2)”, “5alpha-pregnan-3beta,20beta-diol monosulfate (1)”, “5alpha-pregnan-3beta-ol,20-one sulfate”, 6-hydroxyindole sulfate, 6-oxopiperidine-2-carboxylate, 7-alpha-hydroxy-3-oxo-4-cholestenoate (7-Hoca), 7-methylguanine, 7-methylurate, 7-methylxanthine, “9,10-DiHOME”, 9-hydroxystearate, acesulfame, acetoacetate, acetylcarnitine (C2), acisoga, aconitate [cis or trans], adenine, adenosine, adenosine 5′-monophosphate (AMP), adipate, adipoylcarnitine (C6-DC), ADpSGEGDFXAEGGGVR*, adrenate (22:4n6), ADSGEGDFXAEGGGVR*, alanine, allantoin, alliin, alpha-hydroxyisocaproate, alpha-hydroxyisovalerate, alpha-ketobutyrate, alpha-ketoglutarate, alpha-tocopherol, andro steroid monosulfate C19H28O6S (1)*, “androstenediol (3alpha, 17alpha) monosulfate (2)”, “androstenediol (3alpha, 17alpha) monosulfate (3)”, “androstenediol (3beta,17beta) disulfate (1)”, “androstenediol (3beta,17beta) disulfate (2)”, “androstenediol (3beta,17beta) monosulfate (1)”, “androstenediol (3beta,17beta) monosulfate (2)”, androsterone sulfate, anthranilate, arabinose, arabitol/xylitol, arabonate/xylonate, arachidate (20:0), arachidonate (20:4n6), arachidonoylcarnitine (C20:4), arachidonoylcholine, arachidoylcarnitine (C20)*, argininate*, arginine, asparagine, aspartate, atenolol, azelate (nonanedioate), behenoyl dihydrosphingomyelin (d18:0/22:0)*, behenoyl sphingomyelin (d18:1/22:0)*, benzoate, benzoylcarnitine*, beta-alanine, beta-citrylglutamate, beta-cryptoxanthin, beta-hydroxyisovalerate, betaine, “bilirubin (E,E)*”, “bilirubin (E,Z or Z,E)*”, “bilirubin (Z,Z)”, biliverdin, “bradykinin, des-arg(9)”, butyrylcarnitine (C4), C-glycosyltryptophan, caffeic acid sulfate, caffeine, caprate (10:0), caproate (6:0), caprylate (8:0), carboxyethyl-GABA, carboxyibuprofen, carnitine, carotene diol (1), carotene diol (2), carotene diol (3), catechol glucuronide, catechol sulfate, “ceramide (d16:1/24:1, d18:1/22:1)*”, “ceramide (d18:1/14:0, d16:1/16:0)*”, “ceramide (d18:1/20:0, d16:1/22:0, d20:1/18:0)*”, “ceramide (d18:2/24:1, d18:1/24:2)*”, cerotoylcarnitine (C26)*, cetirizine, chenodeoxycholate, chiro-inositol, cholate, cholesterol, choline, choline phosphate, cinnamoylglycine, cis-4-decenoylcarnitine (C10:1), citraconate/glutaconate, citrate, citrulline, corticosterone, cortisol, cortisone, cotinine, cotinine N-oxide, creatine, creatinine, “cys-gly, oxidized”, cystathionine, cysteine, cysteine s-sulfate, cysteine sulfinic acid, cysteine-glutathione disulfide, cysteinylglycine, cystine, cytidine, cytosine, daidzein sulfate (2), decanoylcarnitine (C10), dehydroisoandrosterone sulfate (DHEA-S), deoxycarnitine, deoxycholate, desmethylnaproxen sulfate, dexlansoprazole, dihomo-linoleate (20:2n6), dihomo-linolenate (20:3n3 or n6), dihomo-linolenoyl-choline, dihomo-linolenoylcarnitine (20:3n3 or 6)*, dihomo-linoleoylcarnitine (C20:2)*, dihydroferulic acid, dihydroorotate, dimethyl sulfone, dimethyl sulfoxide (DMSO), dimethylarginine (SDMA+ADMA), dimethylglycine, docosadienoate (22:2n6), docosadioate, docosahexaenoate (DHA; 22:6n3), docosahexaenoylcarnitine (C22:6)*, docosahexaenoylcholine, docosapentaenoate (n3 DPA; 22:5n3), docosapentaenoate (n6 DPA; 22:5n6), docosatrienoate (22:3n3), dodecanedioate, dopamine 3-O-sulfate, dopamine 4-sulfate, DSGEGDFXAEGGGVR*, ectoine, eicosanodioate, eicosapentaenoate (EPA; 20:5n3), eicosapentaenoylcholine, eicosenoate (20:1), eicosenoylcarnitine (C20:1)*, epiandrosterone sulfate, ergothioneine, erucate (22:1n9), erythritol, erythronate*, escitalopram, estrone 3-sulfate, ethyl glucuronide, ethylmalonate, etiocholanolone glucuronide, eugenol sulfate, ferulic acid 4-sulfate, ferulylglycine (1), fexofenadine, fluoxetine, formiminoglutamate, fructose, fumarate, furaneol sulfate, gabapentin, galactonate, gamma-CEHC, gamma-CEHC glucuronide*, gamma-glutamyl-2-aminobutyrate, gamma-glutamyl-alpha-lysine, gamma-glutamyl-epsilon-lysine, gamma-glutamylalanine, gamma-glutamylglutamate, gamma-glutamylglutamine, gamma-glutamylglycine, gamma-glutamylhistidine, gamma-glutamylisoleucine*, gamma-glutamylleucine, gamma-glutamylmethionine, gamma-glutamylphenylalanine, gamma-glutamylthreonine, gamma-glutamyltryptophan, gamma-glutamyltyrosine, gamma-glutamylvaline, gamma-tocopherol/beta-tocopherol, gentisate, gentisic acid-5-glucoside, gluconate, glucose, glucuronate, glutamate, glutamine, glutarate (pentanedioate), glutarylcarnitine (C5-DC), glycerate, glycerol, glycerol 3-phosphate, glycerophosphoethanolamine, glycerophosphoinositol*, glycerophosphorylcholine (GPC), glycine, glycochenodeoxycholate, glycochenodeoxycholate glucuronide (1), glycochenodeoxycholate sulfate, glycocholate, glycocholate glucuronide (1), glycocholenate sulfate*, glycodeoxycholate, glycodeoxycholate glucuronide (1), glycodeoxycholate sulfate, glycohyocholate, glycolithocholate, glycolithocholate sulfate*, “glycosyl ceramide (d18:1/20:0, d16:1/22:0)*”, “glycosyl ceramide (d18:2/24:1, d18:1/24:2)*”, glycosyl-N-(2-hydroxynervonoyl)-sphingosine (d18:1/24:1(2OH))*, glycosyl-N-behenoyl-sphingadienine (d18:2/22:0)*, glycosyl-N-palmitoyl-sphingosine (d18:1/16:0), glycosyl-N-stearoyl-sphingosine (d18:1/18:0), glycoursodeoxycholate, glycylvaline, guanidinoacetate, guanidinosuccinate, guanosine, gulonate*, heneicosapentaenoate (21:5n3), HEPES, heptanoate (7:0), hexadecadienoate (16:2n6), hexadecanedioate, hexanoylcarnitine (C6), hexanoylglutamine, hippurate, histidine, histidylalanine, homoarginine, homocitrulline, homostachydrine*, HWESASXX*, hydantoin-5-propionic acid, hydrochlorothiazide, hydroquinone sulfate, hydroxybupropion, hydroxycotinine, hypotaurine, hypoxanthine, I-urobilinogen, ibuprofen, ibuprofen acyl glucuronide, imidazole lactate, imidazole propionate, indole-3-carboxylic acid, indoleacetate, indoleacetylglutamine, indolelactate, indolepropionate, indolin-2-one, inosine, isobutyrylcarnitine (C4), isocitrate, isoeugenol sulfate, isoleucine, isoursodeoxycholate, isovalerate, isovalerylcarnitine (C5), isovalerylglycine, kynurenate, kynurenine, L-urobilin, lactate, lactose, lactosyl-N-behenoyl-sphingosine (d18:1/22:0)*, lactosyl-N-nervonoyl-sphingosine (d18:1/24:1)*, lactosyl-N-palmitoyl-sphingosine (d18:1/16:0), lanthionine, laurate (12:0), laurylcarnitine (C12), leucine, leucylalanine, leucylglycine, lignoceroyl sphingomyelin (d18:1/24:0), lignoceroylcarnitine (C24)*, linoleamide (18:2n6), linoleate (18:2n6), linolenate [alpha or gamma; (18:3n3 or 6)], linolenoylcarnitine (C18:3)*, linoleoyl ethanolamide, linoleoyl-arachidonoyl-glycerol (18:2/20:4) [1]*, linoleoyl-arachidonoyl-glycerol (18:2/20:4) [2]*, linoleoyl-linoleoyl-glycerol (18:2/18:2) [1]*, linoleoylcarnitine (C18:2)*, linoleoylcholine*, lysine, malate, maleate, malonate, mannitol/sorbitol, mannose, margarate (17:0), margaroylcarnitine*, metformin, methionine, methionine sulfone, methionine sulfoxide, methyl glucopyranoside (alpha+beta),methyl-4-hydroxybenzoate sulfate, methylphosphate, methylsuccinate, methylsuccinoylcarnitine (1), myo-inositol, myristate (14:0), myristoleate (14:1n5), myristoleoylcarnitine (C14:1)*, myristoyl dihydrosphingomyelin (d18:0/14:0)*, myristoylcarnitine (C14), “N,O-didesmethylvenlafaxine glucuronide”, N-(2-furoyl)glycine, N-acetyl-1-methylhistidine*, N-acetyl-3-methylhistidine*, N-acetyl-aspartyl-glutamate (NAAG), N-acetyl-beta-alanine, N-acetyl-cadaverine, N-acetyl-S-allyl-L-cysteine, N-acetylalanine, N-acetylalliin, N-acetylarginine, N-acetylasparagine, N-acetylaspartate (NAA), N-acetylcarnosine, N-acetylcitrulline, N-acetylglucosamine/N-acetylgalactosamine, N-acetylglucosaminylasparagine, N-acetylglutamate, N-acetylglutamine, N-acetylglycine, N-acetylhistidine, N-acetylisoleucine, N-acetylkynurenine (2), N-acetylleucine, N-acetylmethionine, N-acetylmethionine sulfoxide, N-acetylneuraminate, N-acetylphenylalanine, N-acetylproline, N-acetylputrescine, N-acetylserine, N-acetyltaurine, N-acetylthreonine, N-acetyltryptophan, N-acetyltyrosine, N-acetylvaline, N-behenoyl-sphingadienine (d18:2/22:0)*, N-delta-acetylornithine, N-formylanthranilic acid, N-formylmethionine, N-formylphenylalanine, N-methylpipecolate, N-methylproline, N-methyltaurine, N-oleoylserine, N-oleoyltaurine, N-palmitoyl-heptadecasphingosine (d17:1/16:0)*, N-palmitoyl-sphingadienine (d18:2/16:0)*, N-palmitoyl-sphinganine (d18:0/16:0), N-palmitoyl-sphingosine (d18:1/16:0), N-palmitoylglycine, N-palmitoylserine, N-palmitoyltaurine, N-stearoyl-sphingosine (d18:1/18:0)*, N-stearoyltaurine, N-trimethyl 5-aminovalerate, N1-Methyl-2-pyridone-5-carboxamide, N1-methyladenosine, N1-methylinosine, “N2,N2-dimethylguanosine”, “N2,N5-diacetylornithine”, N2-acetyllysine, N4-acetylcytidine, “N6,N6,N6-trimethyllysine”, N6-acetyllysine, N6-carbamoylthreonyladenosine, N6-succinyladenosine, naproxen, naringenin, naringenin 7-glucuronide, nervonoylcarnitine (C24:1)*, nicotinamide, nisinate (24:6n3), nonadecanoate (19:0), norcotinine, norfluoxetine, o-cresol sulfate, O-desmethylvenlafaxine, O-methylcatechol sulfate, O-sulfo-L-tyrosine, octadecanedioate, octanoylcarnitine (C8), oleamide, oleate/vaccenate (18:1), oleoyl ethanolamide, oleoyl-linoleoyl-glycerol (18:1/18:2) [1], oleoyl-linoleoyl-glycerol (18:1/18:2) [2], oleoylcarnitine (C18:1), oleoylcholine, omeprazole, ornithine, orotate, orotidine, oxalate (ethanedioate), oxypurinol, p-cresol sulfate, p-cresol-glucuronide*, palmitate (16:0), palmitic amide, palmitoleate (16:1n7), palmitoleoylcarnitine (C16:1)*, palmitoloelycholine, palmitoyl dihydrosphingomyelin (d18:0/16:0)*, palmitoyl sphingomyelin (d18:1/16:0), palmitoylcarnitine (C16), palmitoylcholine, pantoprazole, pantothenate, paraxanthine, paroxetine, pentadecanoate (15:0), perfluorooctanesulfonic acid (PFOS), phenol glucuronide, phenol sulfate, phenylacetate, phenylacetylcarnitine, phenylacetylglutamine, phenylalanine, phenylalanylglycine, phenyllactate (PLA), phenylpyruvate, phosphate, phosphoethanolamine, phytanate, picolinate, pimeloylcarnitine/3-methyladipoylcarnitine (C7-DC), pipecolate, piperine, pivaloylcarnitine (C5), pregn steroid monosulfate C21H34O5S*, pregnanediol-3-glucuronide, pregnanolone/allopregnanolone sulfate, pregnen-diol disulfate C21H34O8S2*, pregnenolone sulfate, pristanate, pro-hydroxy-pro, proline, prolylglycine, propionylcarnitine (C3), propionylglycine, propyl 4-hydroxybenzoate, propyl 4-hydroxybenzoate sulfate, pseudoephedrine, pseudouridine, pyridostigmine, pyridoxate, pyroglutamine*, pyrraline, pyruvate, quetiapine, quinate, quinine, quinolinate, retinol (Vitamin A), ribitol, riboflavin (Vitamin B2), ribonate, ribose, riluzole, S-1-pyrroline-5-carboxylate, S-adenosylhomocysteine (SAH), S-allylcysteine, S-carboxymethyl-L-cysteine, S-methylcysteine, S-methylcysteine sulfoxide, S-methylmethionine, saccharin, salicylate, salicyluric glucuronide*, sarcosine, sebacate (decanedioate), serine, serotonin, silibinin, sitagliptin, spermidine, sphinganine-1-phosphate, “sphingomyelin (d17:1/16:0, d18:1/15:0, d16:1/17:0)*”, “sphingomyelin (d17:2/16:0, d18:2/15:0)*”, “sphingomyelin (d18:0/18:0, d19:0/17:0)*”, “sphingomyelin (d18:0/20:0, d16:0/22:0)*”, “sphingomyelin (d18:1/14:0, d16:1/16:0)*”, “sphingomyelin (d18:1/17:0, d17:1/18:0, d19:1/16:0)”, “sphingomyelin (d18:1/18:1, d18:2/18:0)”, “sphingomyelin (d18:1/19:0, d19:1/18:0)*”, “sphingomyelin (d18:1/20:0, d16:1/22:0)*”, “sphingomyelin (d18:1/20:1, d18:2/20:0)*”, “sphingomyelin (d18:1/20:2, d18:2/20:1, d16:1/22:2)*”, “sphingomyelin (d18:1/21:0, d17:1/22:0, d16:1/23:0)*”, “sphingomyelin (d18:1/22:1, d18:2/22:0, d16:1/24:1)*”, “sphingomyelin (d18:1/22:2, d18:2/22:1, d16:1/24:2)*”, “sphingomyelin (d18:1/24:1, d18:2/24:0)*”, “sphingomyelin (d18:1/25:0, d19:0/24:1, d20:1/23:0, d19:1/24:0)*”, “sphingomyelin (d18:2/14:0, d18:1/14:1)*”, “sphingomyelin (d18:2/16:0, d18:1/16:1)*”, sphingomyelin (d18:2/18:1)*, “sphingomyelin (d18:2/21:0, d16:2/23:0)*”, “sphingomyelin (d18:2/23:0, d18:1/23:1, d17:1/24:1)*”, sphingomyelin (d18:2/23:1)*, “sphingomyelin (d18:2/24:1, d18:1/24:2)*”, sphingomyelin (d18:2/24:2)*, sphingosine, sphingosine 1-phosphate, stachydrine, stearate (18:0), stearidonate (18:4n3), stearoyl sphingomyelin (d18:1/18:0), stearoylcarnitine (C18), stearoylcholine*, suberate (octanedioate), suberoylcarnitine (C8-DC), succinate, succinylcarnitine (C4-DC), sucrose, sulfate*, syringol sulfate, tartarate, tartronate (hydroxymalonate), taurine, tauro-beta-muricholate, taurochenodeoxycholate, taurocholate, taurocholenate sulfate, taurodeoxycholate, taurolithocholate 3-sulfate, tauroursodeoxycholate, tetradecanedioate, theanine, theobromine, theophylline, thioproline, threonate, threonine, threonylphenylalanine, thymol sulfate, thyroxine, tiglylcarnitine (C5:1-DC), trans-4-hydroxyproline, trans-urocanate, tricosanoyl sphingomyelin (d18:1/23:0)*, triethanolamine, trigonelline (N′-methylnicotinate), trimethylamine N-oxide, tryptophan, tryptophan betaine, tyramine O-sulfate, tyrosine, umbelliferone sulfate, undecanedioate, uracil, urate, urea, uridine, ursodeoxycholate, valerate, valine, valsartan, vanillactate, vanillic alcohol sulfate, vanillylmandelate (VMA), venlafaxine, warfarin, xanthine, xanthosine, xanthurenate, ximenoylcarnitine (C26:1)*, xylose, X-01911, X-07765, X-11261, X-11299, X-11308, X-11315, X-11372, X-11378, X-11381, X-11407, X-11441, X-11442, X-11444, X-11470, X-11478, X-11483, X-11485, X-11491, X-11522, X-11530, X-11593, X-11640, X-11787, X-11795, X-11843, X-11847, X-11849, X-11850, X-11852, X-11858, X-11880, X-12007, X-12013, X-12015, X-12026, X-12063, X-12096, X-12100, X-12101, X-12104, X-12112, X-12117, X-12126, X-12127, X-12193, X-12206, X-12212, X-12216, X-12221, 4-ethylcatechol sulfate, X-12261, X-12263, X-12283, X-12306, X-12329, X-12407, X-12410, X-12411, X-12456, X-12462, X-12472, X-12524, X-12543, X-12544, X-12565, X-12680, X-12701, X-12712, X-12714, X-12718, X-12726, X-12729, X-12730, X-12731, X-12738, X-12739, X-12740, X-12753, X-12798, X-12812, X-12816, X-12818, X-12820, X-12822, X-12830, X-12831, X-12837, X-12839, X-12844, X-12846, X-12847, X-12849, X-12851, X-12879, X-12906, X-13007, X-13255, X-13431, X-13435, X-13553, X-13658, X-13684, X-13703, X-13723, X-13728, X-13729, X-13737, X-13835, X-13844, X-13846, X-13866, X-14056, X-14082, X-14095, X-14096, X-14314, X-14364, X-14662, X-14904, X-14939, X-15220, X-15245, X-15461, X-15469, X-15486, X-15492, X-15503, X-15666, X-15674, X-15728, X-16087, X-16124, X-16132, X-16397, X-16570, X-16576, X-16580, X-16654, X-16935, X-16938, X-16944, X-16946, X-16964, X-17010, X-17145, X-17146, X-17185, X-17325, X-17327, X-17328, X-17335, X-17337, X-17340, X-17343, X-17348, X-17351, X-17353, X-17354, X-17357, X-17359, X-17367, X-17438, X-17469, X-17612, X-17653, X-17654, X-17655, X-17673, X-17676, X-17677, X-17685, X-17690, X-17704, X-17765, X-18240, X-18249, X-18345, X-18606, X-18779, X-18886, X-18887, X-18899, X-18901, X-18913, X-18914, X-18921, X-18922, X-19141, X-19183, X-19434, X-19438, X-19561, X-21258, X-21285, X-21286, X-21295, X-21310, X-21312, X-21319, X-21327, X-21339, X-21341, X-21342, X-21353, X-21364, X-21383, X-21410, X-21411, X-21441, X-21442, X-21444, X-21448, X-21467, X-21470, X-21474, X-21607, X-21628, X-21657, X-21659, X-21661, X-21729, X-21736, X-21737, X-21742, X-21752, X-21792, X-21796, X-21803, X-21807, X-21815, X-21816, X-21821, X-21829, X-21834, X-21838, X-21839, X-21842, X-21845, X-21851, X-22143, X-22162, X-22475, X-22509, X-22520, X-22716, X-22764, X-22771, X-22775, X-22834, X-23276, X-23291, X-23294, X-23295, X-23297, X-23314, X-23369, X-23583, X-23585, X-23587, X-23588, X-23593, X-23637, X-23639, X-23644, X-23649, X-23652, X-23654, X-23655, X-23659, X-23666, X-23680, X-23739, X-23780, X-23782, X-23787, X-23974, X-23997, X-24106, X-24243, X-24293, X-24295, X-24309, X-24328, X-24329, X-24337, X-24348, X-24352, X-24410, X-24411, X-24422, X-24425, X-24432, X-24435, X-24455, X-24456, X-24473, X-24475, X-24498, X-24512, X-24518, X-24519, X-24527, X-24542, X-24544, X-24546, X-24549, X-24550, X-24551, X-24552, X-24554, X-24555, X-24556, X-24557, X-24558, X-24560, X-24571, X-24588, X-24637, X-24655, X-24686, X-24693, X-24699, X-24706, X-24728, X-24736, X-24747, X-24748, X-24757, X-24760, X-24765, X-24801, X-24809, X-24811, X-24812, X-24813, X-24831, X-24832, X-24849, X-24932, X-24947, X-24948, X-24949, X-24951, X-24952, X-24972, X-24983, X-25116, 1-carboxyethylisoleucine, 1-carboxyethylleucine, 1-carboxyethylphenylalanine, 1-carboxyethylvaline, 1-methyl-5-imidazoleacetate, 1-ribosyl-imidazoleacetate*, “2,2′-Methylenebis(6-tert-butyl-p-cresol)”, “2,3-dihydroxy-5-methylthio-4-pentenoate (DMTPA)*”, “2,6-dihydroxybenzoic acid”, 2-naphthol sulfate, 3-(methylthio)acetaminophen sulfate*, 3-amino-2-piperidone, 3-carboxy-4-methyl-5-pentyl-2-furanpropionate (3-CMPFP)**, 3-formylindole, 3-hydroxyhippurate sulfate, 3-hydroxystachydrine*, “5,6-dihydrouridine”, 5-dodecenoylcarnitine (C12:1), 5-methylthioribose**, androsterone glucuronide, cis-4-decenoate (10:1n6)*, cysteinylglycine disulfide*, dihydrocaffeate sulfate (2), dodecadienoate (12:2)*, dodecenedioate (C12:1-DC)*, eicosenedioate (C20:1-DC)*, Fibrinopeptide A (2-15)**, Fibrinopeptide A (3-15)**, Fibrinopeptide A (3-16)**, Fibrinopeptide A (4-15)**, Fibrinopeptide A (5-16)*, Fibrinopeptide A (7-16)*, Fibrinopeptide B (1-11)**, Fibrinopeptide B (1-12)**, Fibrinopeptide B (1-13)**, gamma-glutamylcitrulline*, glu-gly-asn-val**, glucuronide of C10H18O2 (1)*, glucuronide of C10H18O2 (7)*, glucuronide of C10H18O2 (8)*, glycine conjugate of C10H14O2 (1)*, glyco-beta-muricholate**, hexadecenedioate (C16:1-DC)*, hydroxy-CMPF*, “hydroxy-N6,N6,N6-trimethyllysine*”, hydroxyasparagine**, hydroxypalmitoyl sphingomyelin (d18:1/16:0(OH))**, “N,N,N-trimethyl-alanylproline betaine (TMAP)”, “N,N-dimethyl-5-aminovalerate”, N-acetyl-2-aminooctanoate*, N-acetyl-isoputreanine*, N-methylhydroxyproline**, nonanoylcarnitine (C9), octadecadienedioate (C18:2-DC)*, octadecenedioate (C18:1-DC)*, octadecenedioylcarnitine (C18:1-DC)*, perfluorooctanoate (PFOA), picolinoylglycine, pregnenetriol disulfate*, sulfate of piperine metabolite C16H19NO3 (2)*, sulfate of piperine metabolite C16H19NO3 (3)*, taurochenodeoxycholic acid 3-sulfate, taurodeoxycholic acid 3-sulfate, tetradecadienoate (14:2)*, tridecenedioate (C13:1-DC)*

According to a particular embodiment, the metabolite is not glucose and not cholesterol. According to a particular embodiment the metabolite is set forth in Table 1 and more preferably in Table 2. Sequence identifier for the metagenomic sequences of the unknown bacteria recited in Tables 1 and 2 are provided in Table 10.

Lengthy table referenced here
US20220102000A1-20220331-T00001
Please refer to the end of the specification for access instructions.

Lengthy table referenced here
US20220102000A1-20220331-T00002
Please refer to the end of the specification for access instructions.

As used herein, the term “microbiome” refers to the totality of microbes (bacteria, fungae, protists), their genetic elements (genomes) in a defined environment.

According to a particular embodiment, the microbiome is a gut microbiome (i.e. microbiota of the digestive track). In one embodiment, the environment is the small intestine. In another embodiment the environment is the large intestine. The microbiome may be of the lumen or the mucosa of the small intestine or large intestine. In still another embodiment, the gut microbiome is a fecal microbiome.

In some embodiments, a microbiota sample is collected by any means that allows recovery of the microbes and without disturbing the relative amounts of microbes or components or products thereof of a microbiome. In some embodiments, the microbiota sample is a fecal sample. In other embodiments, the microbiota sample is retrieved directly from the gut—e.g. by endoscopy from the lower gastrointestinal (GI) tract or from the upper GI tract. The microbiota sample may be of the lumen of the GI tract or the mucosa of the GI tract.

According to one embodiment the microbiome sample (e.g. fecal sample) is frozen and/or lyophilized prior to analysis. According to another embodiment, the sample may be subjected to solid phase extraction methods.

In some embodiments, the presence, level, and/or activity of between 5 and 10 species of microbes are measured. In some embodiments, the presence, level, and/or activity of between 5 and 20 species of microbes are measured. In some embodiments, the presence, level, and/or activity of between 5 and 50 species of microbes are measured. In some embodiments, the presence, level, and/or activity of between 5 and 100 species of microbes are measured. In some embodiments, the presence, level, and/or activity of between 5 and 500 species of microbes are measured. In some embodiments, the presence, level, and/or activity of between 5 and 1000 species of microbes are measured. In some embodiments, the presence, level, and/or activity of between 50 and 500 species of microbes (e.g. bacteria) are measured. In some embodiments, the presence, level, and/or activity of substantially all species/classes/families of bacteria within the microbiome are measured. In still more embodiments, the presence, level, and/or activity of substantially all the bacteria within the microbiome are measured.

Measuring a level or presence of a microbe may be effected by analyzing for the presence of microbial component or a microbial by-product. Thus, for example the level or presence of a microbe may be effected by measuring the level of a DNA sequence. In some embodiments, the level or presence of a microbe may be effected by measuring 16S rRNA gene sequences or 18S rRNA gene sequences. In other embodiments, the level or presence of a microbe may be effected by measuring RNA transcripts. In still other embodiments the level or presence of a microbe may be effected by measuring proteins. In still other embodiments, the level or presence of a microbe may be effected by measuring metabolites present in the microbiome sample.

Quantifying Microbial Levels:

It will be appreciated that determining the abundance of microbes may be affected by taking into account any feature of the microbiome. Thus, the abundance of microbes may be affected by taking into account the abundance at different phylogenetic levels; at the level of gene abundance; gene metabolic pathway abundances; sub-species strain identification; SNPs and insertions and deletions in specific bacterial regions; growth rates of bacteria, the diversity of the microbes of the microbiome, as further described herein below.

In some embodiments, determining a level or set of levels of one or more types of microbes or components or products thereof comprises determining a level or set of levels of one or more DNA sequences. In some embodiments, one or more DNA sequences comprises any DNA sequence that can be used to differentiate between different microbial types. In certain embodiments, one or more DNA sequences comprises 16S rRNA gene sequences. In certain embodiments, one or more DNA sequences comprises 18S rRNA gene sequences. In some embodiments, 1, 2, 3, 4, 5, 10, 15, 20, 25, 50, 100, 1,000, 5,000 or more sequences are amplified.

16S and 18S rRNA gene sequences encode small subunit components of prokaryotic and eukaryotic ribosomes respectively. rRNA genes are particularly useful in distinguishing between types of microbes because, although sequences of these genes differs between microbial species, the genes have highly conserved regions for primer binding. This specificity between conserved primer binding regions allows the rRNA genes of many different types of microbes to be amplified with a single set of primers and then to be distinguished by amplified sequences.

In some embodiments, a microbiota sample (e.g. fecal sample) is directly assayed for a level or set of levels of one or more DNA sequences. In some embodiments, DNA is isolated from a microbiota sample and isolated DNA is assayed for a level or set of levels of one or more DNA sequences. Methods of isolating microbial DNA are well known in the art. Examples include but are not limited to phenol-chloroform extraction and a wide variety of commercially available kits, including QIAamp DNA Stool Mini Kit (Qiagen, Valencia, Calif.).

In some embodiments, a level or set of levels of one or more DNA sequences is determined by amplifying DNA sequences using PCR (e.g., standard PCR, semi-quantitative, or quantitative PCR) and then sequencing. In some embodiments, a level or set of levels of one or more DNA sequences is determined by amplifying DNA sequences using quantitative PCR. These and other basic DNA amplification procedures are well known to practitioners in the art and are described in Ausebel et al. (Ausubel F M, Brent R, Kingston R E, Moore D, Seidman J G, Smith J A, Struhl K (eds). 1998. Current Protocols in Molecular Biology. Wiley: New York).

In some embodiments, DNA sequences are amplified using primers specific for one or more sequence that differentiate(s) individual microbial types from other, different microbial types. In some embodiments, 16S rRNA gene sequences or fragments thereof are amplified using primers specific for 16S rRNA gene sequences. In some embodiments, 18S DNA sequences are amplified using primers specific for 18S DNA sequences.

In some embodiments, a level or set of levels of one or more 16S rRNA gene sequences is determined using phylochip technology. Use of phylochips is well known in the art and is described in Hazen et al. (“Deep-sea oil plume enriches indigenous oil-degrading bacteria.” Science, 330, 204-208, 2010), the entirety of which is incorporated by reference. Briefly, 16S rRNA genes sequences are amplified and labeled from DNA extracted from a microbiota sample. Amplified DNA is then hybridized to an array containing probes for microbial 16S rRNA genes. Level of binding to each probe is then quantified providing a sample level of microbial type corresponding to 16S rRNA gene sequence probed. In some embodiments, phylochip analysis is performed by a commercial vendor. Examples include but are not limited to Second Genome Inc. (San Francisco, Calif.).

In some embodiments, determining a level or set of levels of one or more types of microbes comprises determining a level or set of levels of one or more microbial RNA molecules (e.g., transcripts). Methods of quantifying levels of RNA transcripts are well known in the art and include but are not limited to northern analysis, semi-quantitative reverse transcriptase PCR, quantitative reverse transcriptase PCR, and microarray analysis.

Methods for sequence determination are generally known to the person skilled in the art. Preferred sequencing methods are next generation sequencing methods or parallel high throughput sequencing methods. For example, a bacterial genomic sequence may be obtained by using Massively Parallel Signature Sequencing (MPSS). An example of an envisaged sequence method is pyrosequencing, in particular 454 pyrosequencing, e.g. based on the Roche 454 Genome Sequencer. This method amplifies DNA inside water droplets in an oil solution with each droplet containing a single DNA template attached to a single primer-coated bead that then forms a clonal colony. Pyrosequencing uses luciferase to generate light for detection of the individual nucleotides added to the nascent DNA, and the combined data are used to generate sequence read-outs. Yet another envisaged example is Illumina or Solexa sequencing, e.g. by using the Illumina Genome Analyzer technology, which is based on reversible dye-terminators. DNA molecules are typically attached to primers on a slide and amplified so that local clonal colonies are formed. Subsequently one type of nucleotide at a time may be added, and non-incorporated nucleotides are washed away. Subsequently, images of the fluorescently labeled nucleotides may be taken and the dye is chemically removed from the DNA, allowing a next cycle. Yet another example is the use of Applied Biosystems' SOLiD technology, which employs sequencing by ligation. This method is based on the use of a pool of all possible oligonucleotides of a fixed length, which are labeled according to the sequenced position. Such oligonucleotides are annealed and ligated. Subsequently, the preferential ligation by DNA ligase for matching sequences typically results in a signal informative of the nucleotide at that position. Since the DNA is typically amplified by emulsion PCR, the resulting bead, each containing only copies of the same DNA molecule, can be deposited on a glass slide resulting in sequences of quantities and lengths comparable to Illumina sequencing. A further method is based on Helicos' Heliscope technology, wherein fragments are captured by polyT oligomers tethered to an array. At each sequencing cycle, polymerase and single fluorescently labeled nucleotides are added and the array is imaged. The fluorescent tag is subsequently removed and the cycle is repeated. Further examples of sequencing techniques encompassed within the methods of the present invention are sequencing by hybridization, sequencing by use of nanopores, microscopy-based sequencing techniques, microfluidic Sanger sequencing, or microchip-based sequencing methods.

According to one embodiment, the sequencing method allows for quantitating the amount of microbe—e.g. by deep sequencing such as Illumina deep sequencing.

As used herein, the term “deep sequencing” refers to a sequencing method wherein the target sequence is read multiple times in the single test. A single deep sequencing run is composed of a multitude of sequencing reactions run on the same target sequence and each, generating independent sequence readout.

In some embodiments, determining a level or set of levels of one or more types of microbes comprises determining a level or set of levels of one or more microbial polypeptides. Methods of quantifying polypeptide levels are well known in the art and include but are not limited to Western analysis and mass spectrometry.

It will be appreciated that although the abundance of any number of microbes may be measured, a limited number are preferably used in the prediction analysis.

The present inventors have shown that the number of microbes whose abundance should be analyzed in order to predict the amount of a blood metabolite may be particular to that metabolite. Preferably, the abundance of at least 5 bacterial species are analyzed, at least 10 bacterial species are analyzed, at least 15 bacterial species are analyzed, at least 20 bacterial species are analyzed, at least 25 bacterial species are analyzed or more than 25 bacterial species are analyzed.

According to another embodiment, in order to classify a microbe as belonging to a particular genus, family, order, class or phylum, it must comprise at least 90% sequence homology, at least 91% sequence homology, at least 92% sequence homology, at least 93% sequence homology, at least 94% sequence homology, at least 95% sequence homology, at least 96% sequence homology, at least 97% sequence homology, at least 98% sequence homology, at least 99% sequence homology to a reference microbe known to belong to the particular genus. According to a particular embodiment, the sequence homology is at least 95%.

According to another embodiment, in order to classify a microbe as belonging to a particular species, it must comprise at least 90% sequence homology, at least 91% sequence homology, at least 92% sequence homology, at least 93% sequence homology, at least 94% sequence homology, at least 95% sequence homology, at least 96% sequence homology, at least 97% sequence homology, at least 98% sequence homology, at least 99% sequence homology to a reference microbe known to belong to the particular species. According to a particular embodiment, the sequence homology is at least 97%.

In determining whether a nucleic acid or protein is substantially homologous or shares a certain percentage of sequence identity with a sequence of the invention, sequence similarity may be defined by conventional algorithms, which typically allow introduction of a small number of gaps in order to achieve the best fit. In particular, “percent identity” of two polypeptides or two nucleic acid sequences is determined using the algorithm of Karlin and Altschul (Proc. Natl. Acad. Sci. USA 87:2264-2268, 1993). Such an algorithm is incorporated into the BLASTN and BLASTX programs of Altschul et al. (J. Mol. Biol. 215:403-410, 1990). BLAST nucleotide searches may be performed with the BLASTN program to obtain nucleotide sequences homologous to a nucleic acid molecule of the invention. Equally, BLAST protein searches may be performed with the BLASTX program to obtain amino acid sequences that are homologous to a polypeptide of the invention. To obtain gapped alignments for comparison purposes, Gapped BLAST is utilized as described in Altschul et al. (Nucleic Acids Res. 25:3389-3402, 1997). When utilizing BLAST and Gapped BLAST programs, the default parameters of the respective programs (e.g., BLASTX and BLASTN) are employed. See www(dot)ncbi(dot)nlm(dot)nih(dot)gov for more details.

In one embodiment, the abundance of no more than 30 bacterial species are analyzed, no more than 40 bacterial species are analyzed or no more than 50 bacterial species are analyzed.

Preferably, at least one of the bacteria that is analyzed belongs to the Clostridiales order.

Preferably at least one of the bacteria that is analyzed belongs to the phylum Firmicutes.

Preferably, at least 20% of the bacteria that are analyzed for the prediction of a single metabolite belong to the phylum Firmicutes. Preferably, at least 30% of the bacteria that are analyzed for the prediction of a single metabolite belong to the phylum Firmicutes. Preferably, at least 40% of the bacteria that are analyzed for the prediction of a single metabolite, belong to the phylum Firmicutes. Preferably, at least 50% of the bacteria that are analyzed for the prediction of a single metabolite belong to the phylum Firmicutes. Preferably, at least 60% of the bacteria that are analyzed for the prediction of a single metabolite belong to the phylum Firmicutes. Preferably, at least 70% of the bacteria that are analyzed for the prediction of a single metabolite belong to the phylum Firmicutes.

In another embodiment, the bacteria that is analyzed does not belong to the Bacteroidetes phylum. Preferably, less than 50% of the bacteria that are analyzed for the prediction of a single metabolite belong to the Bacteroidetes phylum. Preferably, less than 40% of the bacteria that are analyzed for the prediction of a single metabolite belong to the Bacteroidetes phylum. Preferably, less than 30% of the bacteria that are analyzed for the prediction of a single metabolite belong to the Bacteroidetes phylum. Preferably, less than 20% of the bacteria that are analyzed for the prediction of a single metabolite belong to the Bacteroidetes phylum. Preferably, less than 10% of the bacteria that are analyzed for the prediction of a single metabolite belong to the Bacteroidetes phylum.

According to a particular embodiment at least one of the bacterial features whose abundance are analyzed includes: (8002) S: Streptococcus thermophiles; (4810) S: Blautia sp CAG 237; (4961) G: Eubacterium; (3957) F: Lachnospiraceae; (4960) G: Eubacterium; (4581) S: Dorea longicatena; (4782) U: Unknown; (14322) S: Eggerthella sp CAG 209; (5190) S: Firmicutes bacterium CAG 102; (4577) S: Coprococcus comes; (6359) F: Clostridiaceae; (14861) U: Unknown; (3926) U: Unknown; (15073) G: Oscillibacter; (4749) S: Clostridium sp CAG 7; (6148) F: Peptostreptococcaceae; (4705) S: Clostridium sp CAG 43; (14397) S: Collinsella sp CAG 289; (15119) F: Clostridiales unclassified; (15041) F: Clostridiales unclassified; (5843) S: Allisonella histaminiformans; (14921) U: Unknown; (14306) S: Clostridium sp CAG 138; (15154) F: Clostridiales unclassified; (14816) F: Eggerthellaceae.

Table 1 provides a list of preferred bacteria whose abundance may be measured for the quantitative prediction per metabolite.

According to a particular embodiment, the metabolite which is analyzed is set forth in Table 1 and more preferably in Table 2.

The analysis of the amounts of the microbes of the microbiome is optionally and preferably by executing a machine learning procedure.

As used herein the term “machine learning” refers to a procedure embodied as a computer program configured to induce patterns, regularities, or rules from previously collected data to develop an appropriate response to future data, or describe the data in some meaningful way.

Representative examples of machine learning procedures suitable for the present embodiments, include, without limitation, clustering, association rule algorithms, feature evaluation algorithms, subset selection algorithms, support vector machines, classification rules, cost-sensitive classifiers, vote algorithms, stacking algorithms, Bayesian networks, decision trees, neural networks, instance-based algorithms, linear modeling algorithms, k-nearest neighbors (KNN) analysis, ensemble learning algorithms, probabilistic models, graphical models, logistic regression methods (including multinomial logistic regression methods), gradient ascent methods, singular value decomposition methods and principle component analysis.

Following is an overview of some machine learning procedures suitable for the present embodiments.

Support vector machines are algorithms that are based on statistical learning theory. A support vector machine (SVM) according to some embodiments of the present invention can be used for classification purposes and/or for numeric prediction. A support vector machine for classification is referred to herein as “support vector classifier,” support vector machine for numeric prediction is referred to herein as “support vector regression”.

An SVM is typically characterized by a kernel function, the selection of which determines whether the resulting SVM provides classification, regression or other functions. Through application of the kernel function, the SVM maps input vectors into high dimensional feature space, in which a decision hyper-surface (also known as a separator) can be constructed to provide classification, regression or other decision functions. In the simplest case, the surface is a hyper-plane (also known as linear separator), but more complex separators are also contemplated and can be applied using kernel functions. The data points that define the hyper-surface are referred to as support vectors.

The support vector classifier selects a separator where the distance of the separator from the closest data points is as large as possible, thereby separating feature vector points associated with objects in a given class from feature vector points associated with objects outside the class. For support vector regression, a high-dimensional tube with a radius of acceptable error is constructed which minimizes the error of the data set while also maximizing the flatness of the associated curve or function. In other words, the tube is an envelope around the fit curve, defined by a collection of data points nearest the curve or surface.

An advantage of a support vector machine is that once the support vectors have been identified, the remaining observations can be removed from the calculations, thus greatly reducing the computational complexity of the problem. An SVM typically operates in two phases: a training phase and a testing phase. During the training phase, a set of support vectors is generated for use in executing the decision rule. During the testing phase, decisions are made using the decision rule. A support vector algorithm is a method for training an SVM. By execution of the algorithm, a training set of parameters is generated, including the support vectors that characterize the SVM. A representative example of a support vector algorithm suitable for the present embodiments includes, without limitation, sequential minimal optimization.

In KNN analysis, the affinity or closeness of objects is determined. The affinity is also known as distance in a feature space between objects. Based on the determined distances, the objects are clustered and an outlier is detected. Thus, the KNN analysis is a technique to find distance-based outliers based on the distance of an object from its kth-nearest neighbors in the feature space. Specifically, each object is ranked on the basis of its distance to its kth-nearest neighbors. The farthest away object is declared the outlier. In some cases the farthest objects are declared outliers. That is, an object is an outlier with respect to parameters, such as, a k number of neighbors and a specified distance, if no more than k objects are at the specified distance or less from the object. The KNN analysis is a classification technique that uses supervised learning. An item is presented and compared to a training set with two or more classes. The item is assigned to the class that is most common amongst its k-nearest neighbors. That is, compute the distance to all the items in the training set to find the k nearest, and extract the majority class from the k and assign to item.

Association rule algorithm is a technique for extracting meaningful association patterns among features.

The term “association”, in the context of machine learning, refers to any interrelation among features, not just ones that predict a particular class or numeric value. Association includes, but it is not limited to, finding association rules, finding patterns, performing feature evaluation, performing feature subset selection, developing predictive models, and understanding interactions between features.

The term “association rules” refers to elements that co-occur frequently within the datasets. It includes, but is not limited to association patterns, discriminative patterns, frequent patterns, closed patterns, and colossal patterns.

A usual primary step of association rule algorithm is to find a set of items or features that are most frequent among all the observations. Once the list is obtained, rules can be extracted from them.

The aforementioned self-organizing map is an unsupervised learning technique often used for visualization and analysis of high-dimensional data. Typical applications are focused on the visualization of the central dependencies within the data on the map. The map generated by the algorithm can be used to speed up the identification of association rules by other algorithms. The algorithm typically includes a grid of processing units, referred to as “neurons”. Each neuron is associated with a feature vector referred to as observation. The map attempts to represent all the available observations with optimal accuracy using a restricted set of models. At the same time the models become ordered on the grid so that similar models are close to each other and dissimilar models far from each other. This procedure enables the identification as well as the visualization of dependencies or associations between the features in the data.

Feature evaluation algorithms are directed to the ranking of features or to the ranking followed by the selection of features based on their impact.

Information gain is one of the machine learning methods suitable for feature evaluation. The definition of information gain requires the definition of entropy, which is a measure of impurity in a collection of training instances. The reduction in entropy of the target feature that occurs by knowing the values of a certain feature is called information gain. Information gain may be used as a parameter to determine the effectiveness of a feature in explaining the response to the treatment. Symmetrical uncertainty is an algorithm that can be used by a feature selection algorithm, according to some embodiments of the present invention. Symmetrical uncertainty compensates for information gain's bias towards features with more values by normalizing features to a [0,1] range.

Subset selection algorithms rely on a combination of an evaluation algorithm and a search algorithm. Similarly to feature evaluation algorithms, subset selection algorithms rank subsets of features. Unlike feature evaluation algorithms, however, a subset selection algorithm suitable for the present embodiments aims at selecting the subset of features with the highest impact on the metabolite of interest, while accounting for the degree of redundancy between the features included in the subset. The benefits from feature subset selection include facilitating data visualization and understanding, reducing measurement and storage requirements, reducing training and utilization times, and eliminating distracting features to improve classification.

Two basic approaches to subset selection algorithms are the process of adding features to a working subset (forward selection) and deleting from the current subset of features (backward elimination). In machine learning, forward selection is done differently than the statistical procedure with the same name. The feature to be added to the current subset in machine learning is found by evaluating the performance of the current subset augmented by one new feature using cross-validation. In forward selection, subsets are built up by adding each remaining feature in turn to the current subset while evaluating the expected performance of each new subset using cross-validation. The feature that leads to the best performance when added to the current subset is retained and the process continues. The search ends when none of the remaining available features improves the predictive ability of the current subset. This process finds a local optimum set of features.

Backward elimination is implemented in a similar fashion. With backward elimination, the search ends when further reduction in the feature set does not improve the predictive ability of the subset. The present embodiments contemplate search algorithms that search forward, backward or in both directions. Representative examples of search algorithms suitable for the present embodiments include, without limitation, exhaustive search, greedy hill-climbing, random perturbations of subsets, wrapper algorithms, probabilistic race search, schemata search, rank race search, and Bayesian classifier.

A decision tree is a decision support algorithm that forms a logical pathway of steps involved in considering the input to make a decision.

The term “decision tree” refers to any type of tree-based learning algorithms, including, but not limited to, model trees, classification trees, and regression trees.

A decision tree can be used to classify the datasets or their relation hierarchically. The decision tree has tree structure that includes branch nodes and leaf nodes. Each branch node specifies an attribute (splitting attribute) and a test (splitting test) to be carried out on the value of the splitting attribute, and branches out to other nodes for all possible outcomes of the splitting test. The branch node that is the root of the decision tree is called the root node. Each leaf node can represent a classification (e.g., whether a particular input dataset corresponds to a particular metabolite in the subject's blood) or a value (e.g., the predicted quantity of the particular metabolite in the subject's blood). The leaf nodes can also contain additional information about the represented classification such as a confidence score that measures a confidence level in the represented classification (i.e., the likelihood of the classification being accurate). For example, the confidence score can be a continuous value ranging from 0 to 1, in which a score of 0 indicating a very low confidence (e.g., the indication value of the represented classification is very low) and a score of 1 indicating a very high confidence (e.g., the represented classification is almost certainly accurate).

Regression techniques which may be used in accordance with some embodiments the present invention include, but are not limited to linear Regression, Multiple Regression, logistic regression, probit regression, ordinal logistic regression ordinal Probit-Regression, Poisson Regression, negative binomial Regression, multinomial logistic Regression (MLR) and truncated regression.

A logistic regression or logit regression is a type of regression analysis used for predicting the outcome of a categorical dependent variable (a dependent variable that can take on a limited number of values, whose magnitudes are not meaningful but whose ordering of magnitudes may or may not be meaningful) based on one or more predictor variables. Logistic regression may also predict the probability of occurrence for each data point. Logistic regressions also include a multinomial variant. The multinomial logistic regression model is a regression model which generalizes logistic regression by allowing more than two discrete outcomes. That is, it is a model that is used to predict the probabilities of the different possible outcomes of a categorically distributed dependent variable, given a set of independent variables (which may be real-valued, binary-valued, categorical-valued, etc.). For binary-valued variables, a cutoff between the 0 and 1 associations is typically determined using the Yuden Index.

A Bayesian network is a model that represents variables and conditional interdependencies between variables. In a Bayesian network variables are represented as nodes, and nodes may be connected to one another by one or more links. A link indicates a relationship between two nodes. Nodes typically have corresponding conditional probability tables that are used to determine the probability of a state of a node given the state of other nodes to which the node is connected. In some embodiments, a Bayes optimal classifier algorithm is employed to apply the maximum a posteriori hypothesis to a new record in order to predict the probability of its classification, as well as to calculate the probabilities from each of the other hypotheses obtained from a training set and to use these probabilities as weighting factors for future predictions of the subject's blood contents (particularly the metabolites and optionally and preferably their quantity). An algorithm suitable for a search for the best Bayesian network, includes, without limitation, global score metric-based algorithm. In an alternative approach to building the network, Markov blanket can be employed. The Markov blanket isolates a node from being affected by any node outside its boundary, which is composed of the node's parents, its children, and the parents of its children.

Instance-based techniques generate a new model for each instance, instead of basing predictions on trees or networks generated (once) from a training set.

The term “instance”, in the context of machine learning, refers to an example from a dataset.

Instance-based techniques typically store the entire dataset in memory and build a model from a set of records similar to those being tested. This similarity can be evaluated, for example, through nearest-neighbor or locally weighted methods, e.g., using Euclidian distances. Once a set of records is selected, the final model may be built using several different techniques, such as the naive Bayes.

Neural networks are a class of algorithms based on a concept of inter-connected “neurons.” In a typical neural network, neurons contain data values, each of which affects the value of a connected neuron according to connections with pre-defined strengths, and whether the sum of connections to each particular neuron meets a pre-defined threshold. By determining proper connection strengths and threshold values (a process also referred to as training), a neural network can achieve efficient recognition of images and characters. Oftentimes, these neurons are grouped into layers in order to make connections between groups more obvious and to each computation of values. Each layer of the network may have differing numbers of neurons, and these may or may not be related to particular qualities of the input data.

In one implementation, called a fully-connected neural network, each of the neurons in a particular layer is connected to and provides input value to those in the next layer. These input values are then summed and this sum compared to a bias, or threshold. If the value exceeds the threshold for a particular neuron, that neuron then holds a positive value which can be used as input to neurons in the next layer of neurons. This computation continues through the various layers of the neural network, until it reaches a final layer. At this point, the output of the neural network routine can be read from the values in the final layer. Unlike fully-connected neural networks, convolutional neural networks operate by associating an array of values with each neuron, rather than a single value. The transformation of a neuron value for the subsequent layer is generalized from multiplication to convolution.

The machine learning procedure used according to some embodiments of the present invention is a trained machine learning procedure. A machine learning procedure can be trained according to some embodiments of the present invention by feeding a machine learning training program with microbiome data of a cohort of subjects from which the quantities of the metabolite have been determined by blood tests. Once the data are fed, the machine learning training program generates a trained machine learning procedure of a selected type which can then be used without the need to re-train it.

For example, when it is desired to employ decision trees, machine learning training program learns the structure of each tree in a plurality of decision trees (e.g., how many nodes there are in each tree, and how these are connected to one another), and also selects the decision rules for split nodes of each tree. At least a portion of the decision rules relate to one or more microbes in the microbiome. A simple decision rule may be a threshold for the amount of a particular microbes, but more complex rules, relating to more than one microbes are also contemplated. The machine learning training program also accumulates data at the leaves of the trees. The structures of the trees, the decision rules for the split nodes, and the data at the leaves are all selected by the machine learning training program, automatically and typically without user intervention, such that the microbiome data at the root of the trees provide the quantities of the metabolite as determined by blood tests at the leaves of the trees. The final result of the machine learning training program in this case is a set of trees for each metabolite, where the structures, the decision rules for split nodes, and leaf data for each trees are defined by the machine learning training program.

The Examples section that follows describes machine learning training that was used to generate a set of trees for each of a plurality of metabolite, using training data including metabolite quantities and microbiome data collected from a cohort of about 500 subjects.

While the embodiments below are described with a particular emphasis to decision trees, it is to be understood that other types of machine learning procedures can be employed. The skilled person, provided with training data and the description provided herein would know how to train a different type of machine learning procedure to predict the quantity of the metabolite one fed by a plurality of microbes of the microbiome of the subject.

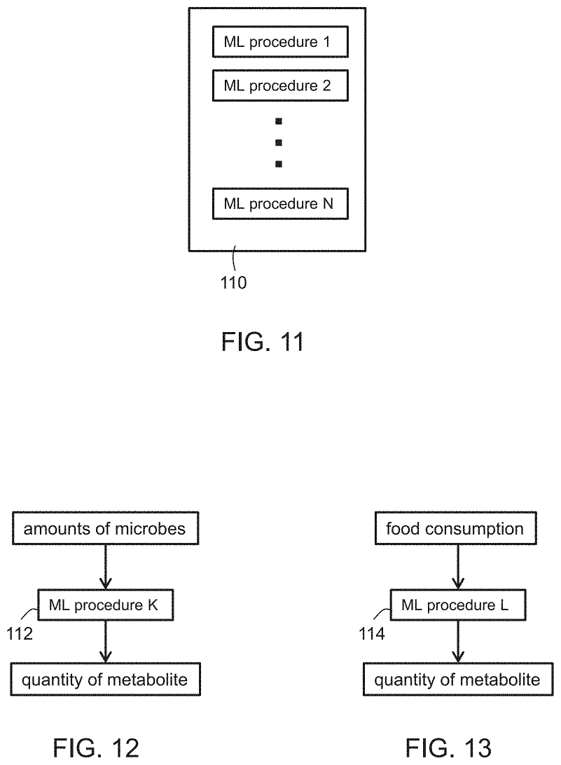

A schematic illustration of the analysis technique according to some embodiments of the present invention is illustrated in FIG. 11. Shown in FIG. 11 is a computer readable medium 110 storing a library of trained machine learning (ML) procedures. Shown are N machine learning (ML) procedures. Typically, each trained machine learning procedures being associated with a different metabolite. Thus, for example, the library can include a machine learning procedure for each of the aforementioned metabolites (in which case N equals the number of the aforementioned metabolites), or a machine learning procedure for each of the metabolites set forth in Table 1 (in which case N equals the number of the metabolites set forth in Table 1), or a machine learning procedure for each of the metabolites set forth in Table 2 (in which case N equals the number of the metabolites set forth in Table 2). Also contemplated are embodiments in which the library includes a machine learning procedure for each of a subset of the aforementioned metabolites or of the metabolites in set forth Table 1, or of the metabolites in set forth Table 2.

The library is accessed and searched for a trained machine learning procedure associated with the metabolite. FIG. 12 illustrates a machine learning procedure 112 which is the Kth (1≤K≤N) procedure in the library, and which is associated with the metabolite of which the quantity in the blood of the subject is to be predicted. The selected trained procedure 112 is fed with the amount of the microbes, and provides an output indicative of the quantity of the metabolite in the blood.

When machine learning procedure 112 includes a set of decision trees, each of the trees receives amounts of microbes, processes these amounts by the split node decision rules that were defined during the training phase, and provides output values in accordance with the data at the leaves that were also defined during the training phase. The output of all trees is optionally and preferably combined (e.g., summed) to provide the quantity of the respective metabolite.

Preferably, the number of trees in the set is at least 1000 or at least 2000 or more. It was found by the inventors that the microbes listed in Table 1 dominate the predicting ability of the decision trees. Thus, in some embodiments of the present invention the number of decision rules relating to microbes listed in Table 1 for the respective metabolite is larger than the number of decision rules relating to other microbes of the microbiome.

According to another aspect of the present invention, there is provided a method of predicting the quantity of a metabolite set forth in Table 1, comprising analyzing the amount of each of the corresponding microbes set forth in Table 1 in the fecal microbiome of the subject, wherein the predicting does not comprise analyzing more than 50 microbes, thereby predicting the quantity of the metabolite in the blood.

Table 1 provides the top five microbes whose abundance should be analyzed in order to predict the quantity of that metabolite.

It will be appreciated that in some cases, additional microbes may be analyzed for each metabolite such that a level of confidence is reached such that the outputted quantities are of clinical relevance e.g. a confidence level of at least 90% and more preferably at least 95%.

As well as using microbial levels to predict the quantity of a blood metabolite, the present inventors further propose using dietary data of the subjects as a proxy for predicting the quantity of a blood metabolite.

Thus, according to another aspect of the present invention there is provided a method of predicting the quantity of a metabolite in the blood of a subject that consumes a diet of a plurality of food types, the method comprising analyzing the frequency of consumption of at least 5 of said food types over at least one month and/or the daily mean consumption of at least 5 of said food types, wherein said frequency and/or said daily mean consumption is predicative, within a confidence level of at least 95% in the significance of the predictions, of the quantity of the metabolite in the blood of the subject consuming said diet.

It will be appreciated that for this aspect of the present invention, the level of a particular metabolite can be predicted in a subject so long as he/she has not significantly changed his/her dietary habits at the time of prediction.

The term “food type” as used herein refers to either a general classification of a food or a particular food product.

In some embodiments of the present invention the food is a food product (e.g., a specific food product marketed as such by a specific manufacturer, or by two or more manufacturers manufacturing the same food product). In some embodiments of the present invention the food is a food type (e.g., a food which exhibit different modifications, for example, white rice, that may have different species, all of which are referred to as “white rice”, or whole wheat bread that may be backed from various mixtures, etc). In some embodiments of the present invention the food is a family of food types. The family can be categorized according to the main ingredient of the food type, for example, sweets, dairies, fruits, herbs, vegetables, fish, meet, etc. In some embodiments of the present invention the family of food types is a food group, such as, but not limited to, carbohydrates, which is a family encompassing food types rich in carbohydrates, proteins, which is a family encompassing food types rich in protein, and fats, which is a family encompassing food types rich in fats, minerals which is a family encompassing food types rich in minerals, vitamins which is a family encompassing food types rich in vitamins, etc. In some embodiments of the present invention the food is a food combination which comprises a plurality of different food products, and/or different food types and/or different food families. Such a combination is referred to as “a complex meal.” The complex meal can be provided as a list of the food products, food types and/or families of food types that form the combination. The list may or may not include the particular amount of each food product, food type and/or family of food types in the combination.

Depending on the particular metabolite being predicted, only the long-term consumption (e.g. over the period of one month) of a particular food type is measured. In another embodiment, only the average daily consumption of a particular food type is measured for predicting the amount of particular metabolites. In other embodiments both the long-term consumption and the average daily consumption is measured.

The information about the subject's food consumption may be obtained by providing the subject with a food questionnaire. The questionnaire may be tailored according to the particular metabolite (or metabolites) which are being investigated. In a particular embodiment, a full survey is obtained from the subject in which the subject is asked to divulge a complete set of food intake per month/per day.

Irrespective of the level of detail the subject is asked to provide with respect to his/her food intake, at least 5 food types are used to predict the level of metabolite. In a particular embodiment, at least 10 food types are used to predict the level of metabolite, at least 15 food types are used to predict the level of metabolite, at least 20 food types are used to predict the level of metabolite, at least 25 food types are used to predict the level of metabolite, at least 30 food types are used to predict the level of metabolite, at least 4 food types are used to predict the level of metabolite, at least 50 food types are used to predict the level of metabolite, or even more than 50 food types are used to predict the level of metabolite. In one embodiment, no more than 50, 60, 70, 80, 90 or 100 food types are used to predict the quantity of a particular metabolite.

The number of food types that are used in the prediction are also dependent on the level of confidence required in the prediction. According to a particular embodiment, the level of confidence is such that the predicted level is clinically relevant. In one embodiment, the prediction is within a confidence level of at least 90%. In another embodiment, the prediction is within a confidence level of at least 95%.

Table 3 herein below, provides exemplary food types that can used to predict particular metabolites.

TABLE 3

| | Top | Directional | Top | Directional | Top | Directional |
|---|---|---|---|---|---|---|
| | predictor | SHAP value | predictor | SHAP value | predictor | SHAP value |
| BIOCHEMICAL | #1 | #1 | #2 | #2 | #3 | #3 |
| X - 16124 | (14816) F: | 0.731921 | (14764) U: | 0.124282 | (14815) F: | 0.120698 |
| | Eggerthellaceae | | Unknown | | Eggerthellaceae | |
| X - 11850 | (14306) S: | 0.377657 | (3926) U: | 0.109301 | (14924) S: | 0.096641 |
| | Clostridium sp | | Unknown | | Firmicutes | |
| | CAG 138 | | | | bacterium | |
| | | | | | CAG 137 | |
| X - 11843 | (14306) S: | 0.354303 | (14924) S: | 0.127773 | (3926) U: | 0.1236 |
| | Clostridium sp | | Firmicutes | | Unknown | |
| | CAG 138 | | bacterium | | | |
| | | | CAG 137 | | | |
| X - 12261 | (3926) U: | 0.165364 | (14306) S: | 0.160851 | (14311) F: | 0.050686 |
| | Unknown | | Clostridium sp | | Clostridiaceae | |
| | | | CAG 138 | | | |
| X - 12013 | (14306) S: | 0.302711 | (3926) U: | 0.138182 | (14924) S: | 0.076535 |
| | Clostridium sp | | Unknown | | Firmicutes | |
| | CAG 138 | | | | bacterium | |
| | | | | | CAG 137 | |
| p-cresol- | (14306) S: | 0.169906 | (15271) S: | 0.110439 | (2328) F: | 0.089761 |
| glucuronide* | Clostridium sp | | Ruthenibacterium | | Rikenellaceae | |
| | CAG 138 | | lactatiformans | | | |
| phenylacetylglutamine | (4951) S: | −0.07214 | (3957) F: | 0.067863 | (15369) S: | 0.048009 |
| | Roseburia | | Lachnospiraceae | | Faecalibacterium sp | |
| | intestinalis | | | | CAG 74 | |
| p-cresol sulfate | (15271) S: | 0.131445 | (14306) S: | 0.092024 | (15236) G: | 0.069206 |
| | Ruthenibacterium | | Clostridium sp | | Firmicutes | |
| | lactatiformans | | CAG 138 | | unclassified | |
| phenylacetate | (3957) F: | 0.083075 | (14306) S: | 0.081132 | (15236) G: | 0.054952 |
| | Lachnospiraceae | | Clostridium sp | | Firmicutes | |
| | | | CAG 138 | | unclassified | |
| X - 12816 | (14921) U: | 0.34516 | (15154) F: | 0.256765 | (15085) F: | 0.075267 |
| | Unknown | | Clostridiales | | Clostridiales | |
| | | | unclassified | | unclassified | |
| quinate | (15154) F: | 0.32007 | (14322) S: | 0.119716 | (4537) S: | −0.06657 |
| | Clostridiales | | Eggerthella sp | | Eubacterium | |
| | unclassified | | CAG 209 | | hallii | |
| 1-methylurate | (15154) F: | 0.354954 | (14921) U: | 0.157361 | (4581) S: | 0.092513 |
| | Clostridiales | | Unknown | | Dorea | |
| | unclassified | | | | longicatena | |
| X - 24811 | (15154) F: | 0.486675 | (14322) S: | 0.13517 | (14921) U: | 0.096585 |
| | Clostridiales | | Eggerthella sp | | Unknown | |
| | unclassified | | CAG 209 | | | |
| 5-acetylamino- | (15154) F: | 0.384464 | (14921) U: | 0.118517 | (4537) S: | −0.07535 |
| 6-amino-3- | Clostridiales | | Unknown | | Eubacterium | |
| methyluracil | unclassified | | | | hallii | |
| 1- | (15154) F: | 0.409986 | (14921) U: | 0.097051 | (4581) S: | 0.072722 |
| methylxanthine | Clostridiales | | Unknown | | Dorea | |
| | unclassified | | | | longicatena | |
| 1,7- | (15154) F: | 0.379716 | (14921) U: | 0.103533 | (4537) S: | −0.07348 |
| dimethylurate | Clostridiales | | Unknown | | Eubacterium | |
| | unclassified | | | | hallii | |
| cinnamoylglycine | (15236) G: | 0.09571 | (15216) F: | 0.071199 | (6140) S: | −0.0711 |
| | Firmicutes | | Clostridiales | | Intestinibacter | |
| | unclassified | | unclassified | | bartlettii | |
| X - 12126 | (15369) S: | 0.116283 | (15234) S: | 0.067535 | (6140) S: | −0.06194 |
| | Faecalibacterium | | Firmicutes | | Intestinibacter | |
| | sp CAG 74 | | bacterium | | bartlettii | |
| | | | CAG 124 | | | |
| 1,3- | (15154) F: | 0.43238 | (14921) U: | 0.085018 | (1861) S: | −0.07465 |
| dimethylurate | Clostridiales | | Unknown | | Bacteroides | |
| | unclassified | | | | thetaiotaomicron | |
| theophylline | (15154) F: | 0.351395 | (14921) U: | 0.105075 | (4537) S: | −0.07713 |
| | Clostridiales | | Unknown | | Eubacterium | |
| | unclassified | | | | hallii | |
| paraxanthine | (15154) F: | 0.48002 | (14322) S: | 0.142044 | (14921) U: | 0.139775 |
| | Clostridiales | | Eggerthella sp | | Unknown | |
| | unclassified | | CAG 209 | | | |
| X - 21442 | (15154) F: | 0.325318 | (14921) U: | 0.149762 | (15295) G: | 0.075169 |
| | Clostridiales | | Unknown | | Gemmiger | |
| | unclassified | | | | | |
| 1,3,7- | (15154) F: | 0.332818 | (14921) U: | 0.100008 | (4537) S: | −0.08922 |
| trimethylurate | Clostridiales | | Unknown | | Eubacterium | |
| | unclassified | | | | hallii | |
| X - 12851 | (4782) U: | 0.141919 | (15346) G: | −0.05185 | (5792) S: | 0.05115 |
| | Unknown | | Faecalibacterium | | Phascolarctobacterium | |
| | | | | | sp CAG | |
| | | | | | 207 | |
| caffeine | (15154) F: | 0.247432 | (4537) S: | −0.08898 | (4960) G: | −0.07476 |
| | Clostridiales | | Eubacterium | | Eubacterium | |
| | unclassified | | hallii | | | |
| X - 12216 | (15369) S: | 0.081022 | (15031) S: | 0.075632 | (3957) F: | 0.060757 |
| | Faecalibacterium | | Firmicutes | | Lachnospiraceae | |
| | sp CAG 74 | | bacterium | | | |
| | | | CAG 110 | | | |
| N-acetyl- | (5843) S: | 0.339842 | (17249) S: | 0.055673 | (15089) S: | 0.042223 |
| cadaverine | Allisonella | | Bifidobacterium | | Firmicutes | |
| | histaminiformans | | longum | | bacterium | |
| | | | | | CAG 83 | |
| 3- | (15236) G: | 0.061088 | (15216) F: | 0.05498 | (15081) F: | 0.042382 |
| phenylpropionate | Firmicutes | | Clostridiales | | Clostridiales | |
| (hydrocinnamate) | unclassified | | unclassified | | unclassified | |
| glycolithocholate | (4552) S: | 0.113145 | (15216) F: | 0.077314 | (4584) S: | −0.06418 |
| sulfate* | Ruminococcus | | Clostridiales | | Ruminococcus | |
| | sp | | unclassified | | gnavus | |
| phenylacetylcarnitine | (14306) S: | 0.10229 | (15356) U: | 0.078615 | (6753) G: | −0.07861 |
| | Clostridium sp | | Unknown | | Clostridium | |
| | CAG 138 | | | | | |
| isoursodeoxycholate | (15265) S: | −0.07283 | (15090) S: | −0.06502 | (15236) G: | −0.06195 |
| | Firmicutes | | Oscillibacter | | Firmicutes | |
| | bacterium | | sp CAG 241 | | unclassified | |
| | CAG 103 | | | | | |
| X - 12837 | (15154) F: | 0.178333 | (6359) F: | 0.162766 | (15106) S: | 0.082873 |
| | Clostridiales | | Clostridiaceae | | Firmicutes | |
| | unclassified | | | | bacterium | |
| | | | | | CAG 176 | |
| X - 24410 | (15119) F: | −0.20813 | (1867) S: | −0.08056 | (1857) S: | −0.05901 |
| | Clostridiales | | Bacteroides | | Bacteroides | |
| | unclassified | | xylanisolvens | | salyersiae | |
| 5alpha- | (14311) F: | 0.099953 | (15244) F: | 0.05353 | (15356) U: | 0.05228 |
| androstan- | Clostridiaceae | | Clostridiales | | Unknown | |
| 3beta,17alpha- | | | unclassified | | | |
| diol disulfate | | | | | | |
| X - 21821 | (4564) S: | −0.06798 | (4940) S: | −0.05164 | (15225) F: | 0.051026 |
| | Ruminococcus | | Roseburia | | Clostridiales | |
| | torques | | inulinivorans | | unclassified | |
| 3-methyl | (15154) F: | 0.195565 | (14861) U: | 0.111519 | (4537) S: | −0.08036 |
| catechol | Clostridiales | | Unknown | | Eubacterium | |
| sulfate (1) | unclassified | | | | hallii | |
| X - 17612 | (3957) F: | 0.086058 | (4940) S: | −0.07886 | (4964) F: | 0.074335 |
| | Lachnospiraceae | | Roseburia | | Eubacteriaceae | |
| | | | inulinivorans | | | |
| 3- | (15154) F: | 0.158727 | (14861) U: | 0.132339 | (4537) S: | −0.06078 |
| hydroxypyridine | Clostridiales | | Unknown | | Eubacterium | |
| sulfate | unclassified | | | | hallii | |
| X - 23655 | (15154) F: | 0.266598 | (14861) U: | 0.135836 | (4961) G: | 0.086895 |
| | Clostridiales | | Unknown | | Eubacterium | |
| | unclassified | | | | | |
| X - 17351 | (4564) S: | −0.08581 | (4940) S: | −0.05069 | (4540) S: | 0.046133 |
| | Ruminococcus | | Roseburia | | Anaerostipes | |
| | torques | | inulinivorans | | hadrus | |
| X - 23997 | (15356) U: | 0.109761 | (15271) S: | 0.097158 | (4951) S: | −0.08016 |
| | Unknown | | Ruthenibacterium | | Roseburia | |
| | | | lactatiformans | | intestinalis | |
| 4- | (14861) U: | 0.13518 | (15154) F: | 0.126693 | (4537) S: | −0.05277 |
| ethylcatechol | Unknown | | Clostridiales | | Eubacterium | |
| sulfate | | | unclassified | | hallii | |
| X - 13729 | (5190) S: | 0.149036 | (3957) F: | 0.116266 | (4571) S: | 0.040898 |
| | Firmicutes | | Lachnospiraceae | | Dorea sp | |
| | bacterium | | | | CAG 105 | |
| | CAG 102 | | | | | |
| ursodeoxycholate | (6148) F: | 0.133438 | (6140) S: | 0.100249 | (4964) F: | −0.09912 |
| | Peptostrep- | | Intestinibacter | | Eubacteriaceae | |
| | tococcaceae | | bartlettii | | | |
| taurolithocholate | (15216) F: | 0.082625 | (14861) U: | 0.050683 | (15356) U: | 0.046204 |
| 3-sulfate | Clostridiales | | Unknown | | Unknown | |
| | unclassified | | | | | |
| X - 17469 | (4552) S: | 0.067777 | (15265) S: | 0.054965 | (4964) F: | 0.054616 |
| | Ruminococcus | | Firmicutes | | Eubacteriaceae | |
| | sp | | bacterium | | | |
| | | | CAG 103 | | | |
| X - 23649 | (15154) F: | 0.214755 | (14861) U: | 0.163407 | (4961) G: | 0.139405 |
| | Clostridiales | | Unknown | | Eubacterium | |
| | unclassified | | | | | |
| 4- | (14397) S: | 0.210295 | (3957) F: | 0.034038 | (15124) F: | 0.028421 |
| methylcatechol | Collinsella sp | | Lachnospiraceae | | Clostridiales | |
| sulfate | CAG 289 | | | | unclassified | |
| indolepropionate | (4810) S: | 0.090297 | (14861) U: | 0.054396 | (4711) F: | 0.049274 |
| | Blautia sp | | Unknown | | Clostridiaceae | |
| | CAG 237 | | | | | |
| citraconate/ | (15154) F: | 0.126893 | (14861) U: | 0.082607 | (4961) G: | 0.078733 |
| glutaconate | Clostridiales | | Unknown | | Eubacterium | |
| | unclassified | | | | | |
| X - 21752 | (6358) S: | 0.070757 | (14311) F: | 0.067583 | (4395) U: | 0.051457 |
| | Clostridium sp | | Clostridiaceae | | Unknown | |
| | CAG 440 | | | | | |
| X - 24243 | (15119) F: | −0.22517 | (4925) S: | 0.057446 | (4771) G: | −0.05377 |
| | Clostridiales | | Roseburia | | Clostridium | |
| | unclassified | | faecis | | | |
| 1-(1-enyl- | (4577) S: | 0.150485 | (6148) F: | 0.074767 | (4960) G: | −0.06689 |
| palmitoyl)-2- | Coprococcus | | Peptostrep- | | Eubacterium | |
| arachidonoyl- | comes | | tococcaceae | | | |
| GPE | | | | | | |
| (P-16:0/20:4)* | | | | | | |
| 5alpha- | (4581) S: | 0.141547 | (4779) S: | 0.105521 | (15120) S: | −0.0944 |
| androstan- | Dorea | | Clostridium sp | | Firmicutes | |
| 3alpha,17beta- | longicatena | | | | bacterium | |
| diol | | | | | CAG 114 | |
| monosulfate | | | | | | |
| (2) | | | | | | |
| hippurate | (14322) S: | 0.086846 | (14861) U: | 0.065422 | (14921) U: | 0.040347 |
| | Eggerthella sp | | Unknown | | Unknown | |
| | CAG 209 | | | | | |
| 5- | (15041) F: | 0.287227 | (15356) U: | 0.110162 | (15042) F: | 0.076708 |
| hydroxyhexanoate | Clostridiales | | Unknown | | Clostridiales | |
| | unclassified | | | | unclassified | |
| indolin-2-one | (3957) F: | 0.085644 | (5190) S: | 0.062315 | (15054) F: | 0.061201 |
| | Lachnospiraceae | | Firmicutes | | Clostridiales | |
| | | | bacterium | | unclassified | |
| | | | CAG 102 | | | |
| X - 17145 | (4564) S: | −0.06692 | (4951) S: | −0.06218 | (15225) F: | 0.051982 |
| | Ruminococcus | | Roseburia | | Clostridiales | |
| | torques | | intestinalis | | unclassified | |
| 2,3- | (15154) F: | 0.289624 | (4537) S: | −0.126 | (14924) S: | −0.1156 |
| dihydroxypyridine | Clostridiales | | Eubacterium | | Firmicutes | |
| | unclassified | | hallii | | bacterium | |
| | | | | | CAG 137 | |
| X - 17354 | (15369) S: | 0.100784 | (4782) U: | 0.089582 | (3940) U: | 0.060603 |
| | Faecalibacterium | | Unknown | | Unknown | |
| | sp CAG 74 | | | | | |
| glycodeoxycholate | (4705) S: | 0.197631 | (4749) S: | 0.173623 | (3957) F: | 0.094735 |
| | Clostridium sp | | Clostridium sp | | Lachnospiraceae | |
| | CAG 43 | | CAG 7 | | | |
| X - 23639 | (15154) F: | 0.162241 | (4714) S: | 0.076215 | (4577) S: | −0.05028 |
| | Clostridiales | | Clostridium sp | | Coprococcus | |
| | unclassified | | | | comes | |
| 6- | (3957) F: | 0.140853 | (5190) S: | 0.077382 | (4581) S: | 0.036247 |
| hydroxyindole | Lachnospiraceae | | Firmicutes | | Dorea | |
| sulfate | | | bacterium | | longicatena | |
| | | | CAG 102 | | | |
| X - 12306 | (4810) S: | 0.135685 | (4960) G: | 0.064322 | (6376) F: | 0.05012 |
| | Blautia sp | | Eubacterium | | Clostridiaceae | |
| | CAG 237 | | | | | |
| phenol sulfate | (4749) S: | 0.062555 | (4788) S: | 0.039635 | (4575) S: | 0.039561 |
| | Clostridium sp | | Firmicutes | | Dorea | |
| | CAG 7 | | bacterium | | formicigenerans | |
| | | | CAG 227 | | | |
| 5-acetylamino- | (15154) F: | 0.299558 | (14322) S: | 0.082648 | (4810) S: | −0.06078 |
| 6-formylamino- | Clostridiales | | Eggerthella sp | | Blautia sp | |
| 3-methyluracil | unclassified | | CAG 209 | | CAG 237 | |
| 1,5- | (15342) S: | 0.096092 | (15154) F: | −0.05172 | (4816) S: | −0.05113 |
| anhydroglucitol | Faecalibacterium | | Clostridiales | | Blautia sp | |
| (1,5-AG) | prausnitzii | | unclassified | | | |
| N- | (4581) S: | 0.086095 | (15216) F: | −0.05304 | (6750) S: | 0.052829 |
| acetylcarnosine | Dorea | | Clostridiales | | Clostridium | |
| | longicatena | | unclassified | | sp | |
| 3-indoxyl | (3957) F: | 0.108384 | (5190) S: | 0.079725 | (4581) S: | 0.042161 |
| sulfate | Lachnospiraceae | | Firmicutes | | Dorea | |
| | | | bacterium | | longicatena | |
| | | | CAG 102 | | | |
| maleate | (14861) U: | 0.080097 | (4961) G: | 0.068112 | (15154) F: | 0.063772 |
| | Unknown | | Eubacterium | | Clostridiales | |
| | | | | | unclassified | |
| L-urobilin | (4425) S: | 0.076548 | (3940) U: | 0.056758 | (15265) S: | 0.051089 |
| | Ruminococcus | | Unknown | | Firmicutes | |
| | sp CAG 254 | | | | bacterium | |
| | | | | | CAG 103 | |
| X - 21286 | (15054) F: | 0.058079 | (4749) S: | −0.05542 | (14252) U: | 0.046933 |
| | Clostridiales | | Clostridium sp | | Unknown | |
| | unclassified | | CAG 7 | | | |
| X - 12718 | (15054) F: | 0.087109 | (3957) F: | 0.056475 | (15089) S: | 0.056144 |
| | Clostridiales | | Lachnospiraceae | | Firmicutes | |
| | unclassified | | | | bacterium | |
| | | | | | CAG 83 | |
| carotene diol | (4810) S: | 0.082594 | (4816) S: | 0.080074 | (4714) S: | 0.062624 |
| (2) | Blautia sp | | Blautia sp | | Clostridium | |
| | CAG 237 | | | | sp | |
| X - 21310 | (3957) F: | 0.104323 | (5190) S: | 0.057593 | (6367) F: | −0.04338 |
| | Lachnospiraceae | | Firmicutes | | Clostridiaceae | |
| | | | bacterium | | | |
| | | | CAG 102 | | | |
| X - 14662 | (6148) F: | 0.055765 | (6140) S: | 0.041721 | (6139) G: | 0.04016 |
| | Peptostrep- | | Intestinibacter | | Intestinibacter | |
| | tococcaceae | | bartlettii | | | |
| glycoursodeoxycholate | (6140) S: | 0.077726 | (15054) F: | −0.05732 | (2325) S: | −0.05048 |
| | Intestinibacter | | Clostridiales | | Alistipes | |
| | bartlettii | | unclassified | | indistinctus | |
| X - 12283 | (4564) S: | −0.08204 | (4608) S: | −0.04699 | (6148) F: | −0.04697 |
| | Ruminococcus | | Ruminococcus | | Peptostrep- | |
| | torques | | torques | | tococcaceae | |
| X - 11315 | (4714) S: | 0.070728 | (4826) S: | −0.04857 | (4810) S: | 0.048092 |
| | Clostridium sp | | Blautia sp | | Blautia sp | |
| | | | | | CAG 237 | |
| trigonelline | (15154) F: | 0.211621 | (14322) S: | 0.083329 | (4961) G: | 0.055074 |
| (N′- | Clostridiales | | Eggerthella sp | | Eubacterium | |
| methylnicotinate) | unclassified | | CAG 209 | | | |
| X - 16654 | (4705) S: | 0.209353 | (4749) S: | 0.121907 | (6962) S: | 0.042728 |
| | Clostridium sp | | Clostridium sp | | Megamonas | |
| | CAG 43 | | CAG 7 | | funiformis | |
| X - 22162 | (15225) F: | 0.080695 | (4867) S: | 0.071521 | (15089) S: | 0.051304 |
| | Clostridiales | | Roseburia sp | | Firmicutes | |
| | unclassified | | CAG 471 | | bacterium | |
| | | | | | CAG 83 | |
| X - 12329 | (14861) U: | 0.108962 | (15154) F: | 0.088758 | (15073) G: | 0.077389 |
| | Unknown | | Clostridiales | | Oscillibacter | |
| | | | unclassified | | | |
| ergothioneine | (4816) S: | 0.062162 | (14991) F: | 0.056879 | (5087) S: | 0.054452 |
| | Blautia sp | | Clostridiaceae | | Eubacterium | |
| | | | | | sp CAG | |
| | | | | | 86 | |
| anthranilate | (3957) F: | 0.097675 | (15369) S: | 0.068549 | (5190) S: | 0.065016 |
| | Lachnospiraceae | | Faecalibacterium | | Firmicutes | |
| | | | sp CAG 74 | | bacterium | |
| | | | | | CAG 102 | |
| cholate | (6148) F: | 0.141591 | (4914) S: | −0.06003 | (6141) F: | 0.055909 |
| | Peptostrep- | | Clostridium sp | | Peptostrep- | |
| | tococcaceae | | | | tococcaceae | |
| 4- | (15369) S: | 0.082561 | (4782) U: | 0.082151 | (2295) S: | 0.058974 |
| hydroxycoumarin | Faecalibacterium | | Unknown | | Alistipes | |
| | sp CAG 74 | | | | shahii | |
| X - 11880 | (17244) S: | 0.125349 | (4940) S: | 0.052936 | (4540) S: | −0.0492 |
| | Bifidobacterium | | Roseburia | | Anaerostipes | |
| | adolescentis | | inulinivorans | | hadrus | |
| X - 22509 | (4782) U: | 0.046536 | (15236) G: | 0.043619 | (4575) S: | −0.03925 |
| | Unknown | | Firmicutes | | Dorea | |
| | | | unclassified | | formicigenerans | |
| 1-lignoceroyl- | (4828) S: | 0.084514 | (4750) G: | −0.05147 | (4705) S: | −0.04686 |
| GPC (24:0) | Blautia sp | | Clostridium | | Clostridium | |
| | | | | | sp CAG | |
| | | | | | 43 | |
| N2,N5- | (4933) S: | −0.06233 | (15132) S: | −0.06226 | (4750) G: | −0.0484 |
| diacetylornithine | Eubacterium | | Flavonifractor | | Clostridium | |
| | rectale | | plautii | | | |
| 3-methyl | (15073) G: | 0.085671 | (15295) G: | 0.082458 | (14861) U: | 0.080126 |
| catechol | Oscillibacter | | Gemmiger | | Unknown | |
| sulfate (2) | | | | | | |
| glutarate | (15119) F: | −0.129 | (4581) S: | 0.043202 | (15154) F: | 0.033058 |
| (pentanedioate) | Clostridiales | | Dorea | | Clostridiales | |
| | unclassified | | longicatena | | unclassified | |
| X - 18249 | (4960) G: | −0.12359 | (8002) S: | 0.08456 | (14861) U: | 0.069294 |
| | Eubacterium | | Streptococcus | | Unknown | |
| | | | thermophilus | | | |
| methyl | (4960) G: | 0.116849 | (4608) S: | −0.05795 | (4867) S: | 0.040669 |
| glucopyranoside | Eubacterium | | Ruminococcus | | Roseburia | |
| (alpha + | | | torques | | sp CAG | |
| beta) | | | | | 471 | |
| 7- | (3957) F: | −0.07918 | (15216) F: | −0.05296 | (17244) S: | 0.052885 |
| methylguanine | Lachnospiraceae | | Clostridiales | | Bifidobacterium | |
| | | | unclassified | | adolescentis | |
| X - 11308 | (4540) S: | −0.06723 | (5736) S: | 0.065334 | (4581) S: | 0.064426 |
| | Anaerostipes | | Acidaminococcus | | Dorea | |
| | hadrus | | intestini | | longicatena | |
| X - 12738 | (4537) S: | −0.09692 | (15073) G: | 0.078229 | (15295) G: | 0.069438 |
| | Eubacterium | | Oscillibacter | | Gemmiger | |
| | hallii | | | | | |
| gentisate | (4957) F: | 0.09566 | (15225) F: | 0.069217 | (4940) S: | −0.06788 |
| | Eubacteriaceae | | Clostridiales | | Roseburia | |
| | | | unclassified | | inulinivorans | |
| carotene diol | (4810) S: | 0.069944 | (15132) S: | −0.06279 | (4714) S: | 0.054082 |
| (1) | Blautia sp | | Flavonifractor | | Clostridium | |
| | CAG 237 | | plautii | | sp | |
| 5alpha- | (4779) S: | 0.12193 | (15120) S: | −0.09663 | (4581) S: | 0.095979 |
| androstan- | Clostridium sp | | Firmicutes | | Dorea | |
| 3alpha,17beta- | | | bacterium | | longicatena | |
| diol disulfate | | | CAG 114 | | | |
| X - 11372 | (17244) S: | 0.100328 | (5736) S: | 0.044797 | (4940) S: | 0.042096 |
| | Bifidobacterium | | Acidaminococcus | | Roseburia | |
| | adolescentis | | intestini | | inulinivorans | |
| X - 17185 | (15154) F: | 0.194347 | (14807) S: | −0.0697 | (1872) S: | −0.06057 |
| | Clostridiales | | Gordonibacter | | Bacteroides | |
| | unclassified | | pamelaeae | | ovatus | |
| X - 23652 | (4577) S: | 0.088679 | (4581) S: | 0.062406 | (6148) F: | 0.05654 |
| | Coprococcus | | Dorea | | Peptostrep- | |
| | comes | | longicatena | | tococcaceae | |
| X - 18240 | (15073) G: | 0.170823 | (1903) S: | 0.059164 | (13982) U: | 0.045312 |
| | Oscillibacter | | Bacteroides | | Unknown | |
| | | | plebeius CAG | | | |
| | | | 211 | | | |
| X - 18914 | (4960) G: | −0.14091 | (8002) S: | 0.133779 | (14921) U: | 0.061579 |
| | Eubacterium | | Streptococcus | | Unknown | |
| | | | thermophilus | | | |
| X - 22520 | (4705) S: | 0.130912 | (4575) S: | 0.070979 | (15265) S: | 0.05918 |
| | Clostridium sp | | Dorea | | Firmicutes | |
| | CAG 43 | | formicigenerans | | bacterium | |
| | | | | | CAG 103 | |
| 3-(3- | (15154) F: | 0.141603 | (15028) G: | −0.06131 | (17248) S: | 0.058498 |
| hydroxyphe- | Clostridiales | | Firmicutes | | Bifidobacterium | |
| nyl)propionate | unclassified | | unclassified | | longum | |
| dimethyl | (4652) S: | 0.064972 | (4940) S: | −0.04917 | (14921) U: | 0.046472 |
| sulfoxide | Clostridium sp | | Roseburia | | Unknown | |
| (DMSO) | CAG 75 | | inulinivorans | | | |
| threonate | (4714) S: | 0.070537 | (4705) S: | −0.0479 | (10068) S: | −0.04741 |
| | Clostridium sp | | Clostridium sp | | Escherichia | |
| | | | CAG 43 | | coli | |
| X - 12730 | (4537) S: | −0.10589 | (14861) U: | 0.086735 | (4961) G: | 0.065742 |
| | Eubacterium | | Unknown | | Eubacterium | |
| | hallii | | | | | |
| X - 19434 | (6148) F: | 0.189861 | (15154) F: | −0.0362 | (4839) G: | 0.033262 |
| | Peptostrep- | | Clostridiales | | Blautia | |
| | tococcaceae | | unclassified | | | |
| X - 24948 | (4940) S: | 0.050366 | (8002) S: | −0.04688 | (4750) G: | 0.044448 |
| | Roseburia | | Streptococcus | | Clostridium | |
| | inulinivorans | | thermophilus | | | |
| 1-(1-enyl- | (4577) S: | 0.120768 | (6148) F: | 0.07976 | (5190) S: | 0.061221 |
| stearoyl)-2- | Coprococcus | | Peptostrep- | | Firmicutes | |
| arachidonoyl- | comes | | tococcaceae | | bacterium | |
| GPE | | | | | CAG 102 | |
| (P-18:0/20:4)* | | | | | | |
| X - 23659 | (4893) S: | 0.07246 | (4816) S: | 0.059256 | (4810) S: | 0.047963 |
| | Clostridium sp | | Blautia sp | | Blautia sp | |
| | | | | | CAG 237 | |
| 5alpha- | (15326) G: | 0.084106 | (4940) S: | 0.064928 | (3964) U: | −0.06018 |
| androstan- | Faecalibacterium | | Roseburia | | Unknown | |
| 3alpha,17alpha- | | | inulinivorans | | | |
| diol | | | | | | |
| monosulfate | | | | | | |
| X - 21339 | (17244) S: | 0.067938 | (4540) S: | −0.06656 | (3964) U: | −0.05966 |
| | Bifidobacterium | | Anaerostipes | | Unknown | |
| | adolescentis | | hadrus | | | |
| 4- | (15370) F: | 0.05494 | (14899) U: | 0.034552 | (4957) F: | 0.031231 |
| ethylphenylsulfate | Ruminococcaceae | | Unknown | | Eubacteriaceae | |
| gamma- | (15078) S: | −0.07302 | (4714) S: | −0.07004 | (4564) S: | 0.065122 |
| glutamylvaline | Oscillibacter | | Clostridium sp | | Ruminococcus | |
| | sp | | | | torques | |
| beta- | (4705) S: | −0.05634 | (15132) S: | −0.0536 | (4575) S: | −0.03664 |
| cryptoxanthin | Clostridium sp | | Flavonifractor | | Dorea | |
| | CAG 43 | | plautii | | formicigenerans | |
| sphingomyelin | (8002) S: | 0.10129 | (14921) U: | 0.052085 | (15271) S: | 0.041212 |
| (d18:1/14:0, | Streptococcus | | Unknown | | Ruthenibacterium | |
| d16:1/16:0)* | thermophilus | | | | lactatiformans | |
| X - 21736 | (4940) S: | 0.054973 | (14823) F: | 0.051274 | (17248) S: | −0.04421 |
| | Roseburia | | Eggerthellaceae | | Bifidobacterium | |
| | inulinivorans | | | | longum | |
| O-methylcatechol | (4537) S: | −0.09144 | (15154) F: | 0.073441 | (14322) S: | 0.057463 |
| sulfate | Eubacterium | | Clostridiales | | Eggerthella | |
| | hallii | | unclassified | | sp CAG | |
| | | | | | 209 | |
| N-(2- | (14861) U: | 0.071344 | (15154) F: | 0.068474 | (15295) G: | 0.066258 |
| furoyl)glycine | Unknown | | Clostridiales | | Gemmiger | |
| | | | unclassified | | | |
| sphingomyelin | (4714) S: | −0.05271 | (5736) S: | −0.05148 | (15373) F: | 0.050773 |
| (d17:2/16:0, | Clostridium sp | | Acidaminococcus | | Ruminococcaceae | |
| d18:2/15:0)* | | | intestini | | | |
| 3- | (4577) S: | 0.098923 | (6148) F: | 0.038941 | (5190) S: | 0.031545 |
| methylhistidine | Coprococcus | | Peptostrep- | | Firmicutes | |
| | comes | | tococcaceae | | bacterium | |
| | | | | | CAG 102 | |
| X - 13835 | (4577) S: | 0.129616 | (4581) S: | 0.086872 | (6148) F: | 0.083047 |
| | Coprococcus | | Dorea | | Peptostrep- | |
| | comes | | longicatena | | tococcaceae | |
| propionylcarnitine | (15286) F: | −0.0589 | (4575) S: | 0.056616 | (6179) G: | 0.05558 |
| (C3) | Ruminococcaceae | | Dorea | | Clostridium | |
| | | | formicigenerans | | | |
| 3- | (15154) F: | 0.097476 | (4537) S: | −0.04878 | (6359) F: | 0.042861 |
| hydroxyhippurate | Clostridiales | | Eubacterium | | Clostridiaceae | |
| | unclassified | | hallii | | | |
| X - 11640 | (4782) U: | 0.065486 | (2295) S: | 0.027253 | (15229) F: | 0.02622 |
| | Unknown | | Alistipes | | Clostridiales | |
| | | | shahii | | unclassified | |
| 3-acetylphenol | (4537) S: | −0.1503 | (4961) G: | 0.10868 | (4953) S: | −0.09756 |
| sulfate | Eubacterium | | Eubacterium | | Roseburia | |
| | hallii | | | | sp CAG | |
| | | | | | 182 | |
| myo-inositol | (3574) U: | 0.058789 | (17244) S: | −0.05649 | (6754) S: | 0.053803 |
| | Unknown | | Bifidobacterium | | Clostridium sp | |
| | | | adolescentis | | | |
| sphingomyelin | (15271) S: | 0.043125 | (4540) S: | 0.040324 | (5803) S: | −0.037 |
| (d18:2/23:1)* | Ruthenibacterium | | Anaerostipes | | Dialister sp | |
| | lactatiformans | | hadrus | | CAG 357 | |
| 2-naphthol | (15350) U: | 0.090311 | (14861) U: | 0.085983 | (15132) S: | 0.055031 |
| sulfate | Unknown | | Unknown | | Flavonifractor | |
| | | | | | plautii | |
| N-delta- | (5087) S: | 0.045935 | (4960) G: | 0.035767 | (9283) S: | 0.027156 |
| acetylornithine | Eubacterium | | Eubacterium | | Sutterella | |
| | sp CAG 86 | | | | wadsworthensis | |
| benzoylcarnitine* | (15081) F: | 0.071059 | (4648) G: | 0.049972 | (14322) S: | 0.048698 |
| | Clostridiales | | Roseburia | | Eggerthella | |
| | unclassified | | | | sp CAG | |
| | | | | | 209 | |
| X - 24473 | (4960) G: | 0.085911 | (4714) S: | 0.069271 | (4269) S: | 0.050226 |
| | Eubacterium | | Clostridium sp | | Clostridium sp | |
| X - 11381 | (8002) S: | 0.055129 | (4714) S: | −0.04985 | (4960) G: | −0.04506 |
| | Streptococcus | | Clostridium sp | | Eubacterium | |
| | thermophilus | | | | | |
| X - 22834 | (4705) S: | 0.159163 | (4824) G: | 0.078445 | (1877) S: | −0.05965 |
| | Clostridium sp | | Blautia | | Bacteroides | |
| | CAG 43 | | | | caccae | |
| oxalate | (4705) S: | −0.06967 | (6754) S: | 0.06754 | (14909) S: | −0.05608 |
| (ethanedioate) | Clostridium sp | | Clostridium sp | | Clostridium | |
| | CAG 43 | | | | sp CAG | |
| | | | | | 169 | |
| alpha- | (4940) S: | 0.060534 | (4951) S: | 0.057954 | (1814) S: | 0.05535 |
| hydroxyisovalerate | Roseburia | | Roseburia | | Bacteroides | |
| | inulinivorans | | intestinalis | | vulgatus | |
| X - 24693 | (4581) S: | −0.05561 | (4810) S: | 0.051504 | (8002) S: | −0.04807 |
| | Dorea | | Blautia sp | | Streptococcus | |
| | longicatena | | CAG 237 | | thermophilus | |
| X - 24736 | (4960) G: | 0.128623 | (14991) F: | 0.089667 | (4810) S: | 0.08736 |
| | Eubacterium | | Clostridiaceae | | Blautia sp | |
| | | | | | CAG 237 | |
| 1H-indole-7- | (4804) S: | 0.076411 | (15225) F: | 0.073963 | (3952) U: | 0.062717 |
| acetic acid | Blautia sp | | Clostridiales | | Unknown | |
| | | | unclassified | | | |
| urate | (9226) S: | −0.04466 | (4540) S: | −0.04131 | (4705) S: | 0.038998 |
| | Akkermansia | | Anaerostipes | | Clostridium | |
| | muciniphila | | hadrus | | sp CAG | |
| | | | | | 43 | |
| taurodeoxycholate | (4705) S: | 0.117422 | (5785) S: | 0.05815 | (6148) F: | −0.05486 |
| | Clostridium sp | | Phascolarctobacterium | | Peptostrep- | |
| | CAG 43 | | sp | | tococcaceae | |
| | | | CAG 266 | | | |
| sphingomyelin | (15373) F: | 0.074581 | (4564) S: | −0.04682 | (5736) S: | −0.0443 |
| (d18:2/14:0, | Ruminococcaceae | | Ruminococcus | | Acidaminococcus | |
| d18:1/14:)* | | | torques | | intestini | |
| glycolithocholate | (6747) S: | −0.09414 | (4425) S: | 0.041059 | (4940) S: | −0.04097 |
| | Clostridium | | Ruminococcus | | Roseburia | |
| | spiroforme | | sp CAG 254 | | inulinivorans | |
| X - 15728 | (15322) S: | 0.063863 | (14311) F: | 0.042364 | (4957) F: | 0.041103 |
| | Faecalibacterium | | Clostridiaceae | | Eubacteriaceae | |
| | prausnitzii | | | | | |
| creatinine | (6750) S: | 0.092459 | (4581) S: | 0.058309 | (4820) S: | −0.04065 |
| | Clostridium sp | | Dorea | | Blautia sp | |
| | | | longicatena | | | |
| X - 15461 | (5843) S: | 0.121367 | (4581) S: | 0.071379 | (1872) S: | −0.03418 |
| | Allisonella | | Dorea | | Bacteroides | |
| | histaminiformans | | longicatena | | ovatus | |
| X - 12822 | (6796) G: | −0.15731 | (6806) S: | −0.08175 | (4871) S: | 0.04609 |
| | Holdemanella | | Holdemanella | | Ruminococcus | |
| | | | biformis | | sp | |
| 4-allylphenol | (6362) S: | −0.05081 | (4781) U: | 0.046877 | (4826) S: | −0.04474 |
| sulfate | Clostridium sp | | Unknown | | Blautia sp | |
| | CAG 343 | | | | | |
| X - 23782 | (14974) U: | 0.063947 | (15154) F: | 0.045085 | (14400) G: | −0.03917 |
| | Unknown | | Clostridiales | | Collinsella | |
| | | | unclassified | | | |
| X - 12212 | (9262) S: | −0.14662 | (4834) G: | 0.03958 | (4810) S: | 0.039211 |
| | Burkholderiales | | Blautia | | Blautia sp | |
| | bacterium | | | | CAG 237 | |
| | 1 1 47 | | | | | |
| tryptophan | (4564) S: | −0.04635 | (4714) S: | 0.039997 | (14861) U: | −0.03518 |
| betaine | Ruminococcus | | Clostridium sp | | Unknown | |
| | torques | | | | | |
| I-urobilinogen | (2318) S: | −0.04726 | (4198) S: | −0.04003 | (15249) S: | −0.03252 |
| | Alistipes | | Eubacterium | | Firmicutes | |
| | putredinis | | siraeum | | bacterium | |
| | | | | | CAG 129 | |
| sphingomyelin | (15154) F: | 0.047126 | (4714) S: | −0.04651 | (8002) S: | 0.033388 |
| (d18:1/19:0, | Clostridiales | | Clostridium sp | | Streptococcus | |
| d19:1/18:0)* | unclassified | | | | thermophilus | |
| 3-carboxy-4- | (4839) G: | 0.080966 | (4828) S: | 0.027953 | (17244) S: | −0.02581 |
| methyl-5- | Blautia | | Blautia sp | | Bifidobacterium | |
| pentyl-2- | | | | | adolescentis | |
| furanpropionate | | | | | | |
| (3-CMPFP)** | | | | | | |
| X - 16935 | (17244) S: | 0.083128 | (3964) U: | −0.0762 | (4581) S: | 0.054756 |
| | Bifidobacterium | | Unknown | | Dorea | |
| | adolescentis | | | | longicatena | |
| sphingomyelin | (14921) U: | 0.064692 | (4714) S: | −0.04131 | (15271) S: | 0.035821 |
| (d17:1/16:0, | Unknown | | Clostridium sp | | Ruthenibacterium | |
| d18:1/15:0, | | | | | lactatiformans | |
| d16:1/17:0)* | | | | | | |
| X - 21829 | (4940) S: | 0.088208 | (14823) F: | 0.058811 | (14993) S: | 0.043001 |
| | Roseburia | | Eggerthellaceae | | Butyricicoccus | |
| | inulinivorans | | | | sp | |
| cystine | (1836) S: | −0.08619 | (6796) G: | 0.046165 | (15216) F: | −0.02599 |
| | Bacteroides | | Holdemanella | | Clostridiales | |
| | uniformis | | | | unclassified | |
| X - 24475 | (4964) F: | 0.092409 | (4810) S: | 0.03616 | (4197) G: | 0.031048 |
| | Eubacteriaceae | | Blautia sp | | Ruminiclostridium | |
| | | | CAG 237 | | | |
| 1-stearoyl-2- | (1862) S: | 0.046774 | (15295) G: | 0.035784 | (4658) S: | 0.029873 |
| docosahexaenoyl-GPC | Bacteroides | | Gemmiger | | Clostridium | |
| (18:0/22:6) | finegoldii | | | | sp CAG | |
| | | | | | 253 | |
| X - 24951 | (4540) S: | −0.04655 | (4581) S: | 0.043836 | (17244) S: | 0.043256 |
| | Anaerostipes | | Dorea | | Bifidobacterium | |
| | hadrus | | longicatena | | adolescentis | |
| X - 24949 | (4936) S: | 0.071924 | (8002) S: | −0.05766 | (14861) U: | −0.05612 |
| | Roseburia | | Streptococcus | | Unknown | |
| | hominis | | thermophilus | | | |
| 2- | (3964) U: | −0.04587 | (4540) S: | −0.04577 | (17244) S: | 0.03493 |
| hydroxylaurate | Unknown | | Anaerostipes | | Bifidobacterium | |
| | | | hadrus | | adolescentis | |
| X - 12063 | (4705) S: | 0.107575 | (4644) S: | −0.06968 | (6376) F: | −0.05699 |
| | Clostridium sp | | Clostridium sp | | Clostridiaceae | |
| | CAG 43 | | CAG 62 | | | |
| 2-hydroxy-3- | (4940) S: | 0.052212 | (1814) S: | 0.035716 | (4564) S: | 0.027496 |
| methylvalerate | Roseburia | | Bacteroides | | Ruminococcus | |
| | inulinivorans | | vulgatus | | torques | |
| argininate* | (15132) S: | −0.06754 | (4953) S: | 0.063846 | (4811) S: | −0.05058 |
| | Flavonifractor | | Roseburia sp | | Blautia | |
| | plautii | | CAG 182 | | obeum | |
| indoleacetate | (3926) U: | 0.092723 | (14899) U: | 0.026394 | (4933) S: | −0.02197 |
| | Unknown | | Unknown | | Eubacterium | |
| | | | | | rectale | |
| ceramide | (8002) S: | 0.122669 | (15154) F: | 0.088126 | (15315) G: | −0.05644 |
| (d18:1/14:0, | Streptococcus | | Clostridiales | | Faecalibacterium | |
| d16:1/16:0)* | thermophilus | | unclassified | | | |
| 5alpha- | (15120) S: | −0.04398 | (4581) S: | 0.042816 | (4303) S: | 0.041358 |
| androstan- | Firmicutes | | Dorea | | Clostridium | |
| 3beta,17beta- | bacterium | | longicatena | | sp CAG | |
| diol disulfate | CAG 114 | | | | 217 | |
| citrulline | (4930) F: | 0.036932 | (5082) S: | −0.03622 | (15272) F: | −0.03503 |
| | Lachnospiraceae | | Eubacterium | | Ruminococcaceae | |
| | | | eligens | | | |
| 1-methyl-5- | (4577) S: | 0.100013 | (4581) S: | 0.05893 | (6148) F: | 0.044928 |
| imidazoleacetate | Coprococcus | | Dorea | | Peptostrep- | |
| | comes | | longicatena | | tococcaceae | |
| X - 12263 | (15154) F: | 0.115405 | (14322) S: | 0.06079 | (1872) S: | −0.05794 |
| | Clostridiales | | Eggerthella sp | | Bacteroides | |
| | unclassified | | CAG 209 | | ovatus | |
| taurodeoxycholic | (6148) F: | −0.0488 | (15143) S: | 0.046284 | (15078) S: | 0.034546 |
| acid 3- | Peptostrep- | | Flavonifractor | | Oscillibacter sp | |
| sulfate | tococcaceae | | sp | | | |
| X - 12543 | (15154) F: | 0.128502 | (15028) G: | −0.05789 | (4771) G: | −0.04424 |
| | Clostridiales | | Firmicutes | | Clostridium | |
| | unclassified | | unclassified | | | |
| sphingomyelin | (15154) F: | 0.047841 | (15373) F: | 0.045007 | (15271) S: | 0.043435 |
| (d18:2/21:0, | Clostridiales | | Ruminococcaceae | | Ruthenibacterium | |
| d16:2/23:0)* | unclassified | | | | lactatiformans | |
| N- | (6179) G: | 0.033585 | (2303) S: | 0.025064 | (6750) S: | 0.015072 |
| acetylmethionine | Clostridium | | Alistipes | | Clostridium sp | |
| | | | finegoldii | | | |
| X - 18901 | (15385) U: | 0.033368 | (4782) U: | 0.023265 | (6422) S: | 0.020133 |
| | Unknown | | Unknown | | Clostridium | |
| | | | | | sp CAG | |
| | | | | | 433 | |
| 1- | (15132) S: | 0.080403 | (5075) S: | 0.071374 | (15216) F: | −0.05975 |
| palmitoylglycerol | Flavonifractor | | Lachnospira | | Clostridiales | |
| (16:0) | plautii | | pectinoschiza | | unclassified | |
| X - 23587 | (4706) F: | 0.052422 | (15031) S: | −0.05126 | (6140) S: | −0.04005 |
| | Clostridiaceae | | Firmicutes | | Intestinibacter | |
| | | | bacterium | | bartlettii | |
| | | | CAG 110 | | | |
| androstenediol | (4581) S: | 0.039049 | (4940) S: | 0.036934 | (5736) S: | 0.027905 |
| (3beta,17beta) | Dorea | | Roseburia | | Acidaminococcus | |
| disulfate (2) | longicatena | | inulinivorans | | intestini | |
| tartronate | (4705) S: | −0.08833 | (6754) S: | 0.059431 | (3988) F: | −0.04224 |
| (hydroxymalonate) | Clostridium sp | | Clostridium sp | | Firmicutes | |
| | CAG 43 | | | | unclassified | |
| X - 24352 | (4964) F: | 0.064927 | (4953) S: | 0.043616 | (4269) S: | 0.03609 |
| | Eubacteriaceae | | Roseburia sp | | Clostridium sp | |
| | | | CAG 182 | | | |
| X - 23654 | (1812) S: | 0.086706 | (15286) F: | −0.08556 | (10068) S: | −0.04185 |
| | Bacteroides | | Ruminococcaceae | | Escherichia | |
| | massiliensis | | | | coli | |
| dihydrocaffeate | (15154) F: | 0.136675 | (15225) F: | −0.0452 | (4029) U: | 0.041803 |
| sulfate (2) | Clostridiales | | Clostridiales | | Unknown | |
| | unclassified | | unclassified | | | |
| sphingomyelin | (15154) F: | 0.054011 | (4714) S: | −0.05102 | (14921) U: | 0.039123 |
| (d18:1/17:0, | Clostridiales | | Clostridium sp | | Unknown | |
| d17:1/18:0, | unclassified | | | | | |
| d19:1/16:0) | | | | | | |
| 3-carboxy-4- | (15332) S: | 0.040653 | (17239) S: | −0.03551 | (4810) S: | 0.033552 |
| methyl-5- | Faecalibacterium | | Bifidobacterium | | Blautia sp | |
| propyl-2- | prausnitzii | | sp N4G05 | | CAG 237 | |
| furanpropanoate | | | | | | |
| (CMPF) | | | | | | |
| X - 18606 | (14991) F: | 0.075629 | (15216) F: | −0.03112 | (6174) S: | 0.029137 |
| | Clostridiaceae | | Clostridiales | | Clostridium | |
| | | | unclassified | | sp CAG | |
| | | | | | 265 | |
| 2,3-dihydroxy- | (4608) S: | −0.07499 | (4810) S: | 0.063775 | (4811) S: | −0.0333 |
| 2-methylbutyrate | Ruminococcus | | Blautia sp | | Blautia | |
| | torques | | CAG 237 | | obeum | |
| X - 12221 | (4960) G: | −0.08349 | (14861) U: | 0.058238 | (6173) S: | 0.052317 |
| | Eubacterium | | Unknown | | Clostridium | |
| | | | | | sp CAG | |
| | | | | | 221 | |
| X - 14082 | (4961) G: | 0.082112 | (15154) F: | 0.066348 | (14861) U: | 0.04638 |
| | Eubacterium | | Clostridiales | | Unknown | |
| | | | unclassified | | | |
| X - 13703 | (14322) S: | 0.05466 | (4961) G: | 0.046668 | (15073) G: | 0.041537 |
| | Eggerthella sp | | Eubacterium | | Oscillibacter | |
| | CAG 209 | | | | | |
| X - 17676 | (14861) U: | 0.11075 | (4537) S: | −0.07179 | (15154) F: | 0.065636 |
| | Unknown | | Eubacterium | | Clostridiales | |
| | | | hallii | | unclassified | |
| X - 24801 | (4705) S: | 0.054674 | (14894) S: | −0.02865 | (2303) S: | −0.0282 |
| | Clostridium sp | | Anaeromassili | | Alistipes | |
| | CAG 43 | | bacillus sp | | finegoldii | |
| | | | An250 | | | |
| N- | (4960) G: | 0.127868 | (4582) S: | −0.03177 | (4581) S: | 0.030253 |
| methylproline | Eubacterium | | Dorea | | Dorea | |
| | | | longicatena | | longicatena | |
| 1-(1-enyl- | (4577) S: | 0.092485 | (5190) S: | 0.068759 | (6148) F: | 0.040227 |
| palmitoyl)-2- | Coprococcus | | Firmicutes | | Peptostrep- | |
| linoleoyl-GPE | comes | | bacterium | | tococcaceae | |
| (P-16:0/18:2)* | | | CAG 102 | | | |
| sphingomyelin | (15373) F: | 0.049768 | (5736) S: | −0.04939 | (15266) G: | −0.04526 |
| (d18:2/23:0, | Ruminococcaceae | | Acidaminococcus | | Firmicutes | |
| d18:1/23:1, | | | intestini | | unclassified | |
| d17:1/24:1)* | | | | | | |
| eicosenedioate | (17244) S: | 0.04508 | (3964) U: | −0.03989 | (4540) S: | −0.03295 |
| (C20:1-DC)* | Bifidobacterium | | Unknown | | Anaerostipes | |
| | adolescentis | | | | hadrus | |
| picolinoylglycine | (8002) S: | 0.071639 | (6179) G: | 0.067192 | (4577) S: | 0.062939 |
| | Streptococcus | | Clostridium | | Coprococcus | |
| | thermophilus | | | | comes | |
| 5alpha- | (4940) S: | 0.067331 | (15326) G: | 0.041349 | (3964) U: | −0.03758 |
| androstan- | Roseburia | | Faecalibacterium | | Unknown | |
| 3alpha,17beta- | inulinivorans | | | | | |
| diol | | | | | | |
| monosulfate | | | | | | |
| (1) | | | | | | |
| S- | (15078) S: | −0.03677 | (5843) S: | −0.03242 | (14594) G: | −0.03007 |
| methylmethionine | Oscillibacter | | Allisonella | | Collinsella | |
| | sp | | histaminiformans | | | |
| glycocholate | (17237) S: | −0.06445 | (15164) F: | 0.060041 | (4782) U: | 0.055125 |
| glucuronide (1) | Bifidobacterium | | Clostridiales | | Unknown | |
| | pseudocatenulatum | | unclassified | | | |
| 1- | (1786) S: | 0.060988 | (15332) S: | 0.059863 | (3957) F: | −0.04925 |
| docosahexaenoylglycerol | Butyricimonas | | Faecalibacterium | | Lachnospiraceae | |
| (22:6) | synergistica | | prausnitzii | | | |
| dodecanedioate | (14921) U: | 0.054548 | (15395) U: | 0.039669 | (15132) S: | 0.034876 |
| | Unknown | | Unknown | | Flavonifractor | |
| | | | | | plautii | |
| androstenediol | (4940) S: | 0.058422 | (3964) U: | −0.0375 | (4564) S: | 0.033429 |
| (3beta,17beta) | Roseburia | | Unknown | | Ruminococcus | |
| monosulfate | inulinivorans | | | | torques | |
| (1) | | | | | | |
| X - 16087 | (4839) G: | 0.082987 | (14400) G: | −0.07288 | (15322) S: | 0.047156 |
| | Blautia | | Collinsella | | Faecalibacterium | |
| | | | | | prausnitzii | |
| S- | (4652) S: | 0.047736 | (6139) G: | 0.025248 | (4584) S: | −0.02511 |
| methylcysteine | Clostridium sp | | Intestinibacter | | Ruminococcus | |
| sulfoxide | CAG 75 | | | | gnavus | |
| X - 23314 | (4960) G: | 0.115378 | (15452) S: | −0.02867 | (4714) S: | 0.025175 |
| | Eubacterium | | Bilophila sp 4 | | Clostridium sp | |
| | | | 1 30 | | | |
| N1- | (4960) G: | −0.10761 | (4644) S: | −0.02687 | (1872) S: | −0.02297 |
| methylinosine | Eubacterium | | Clostridium sp | | Bacteroides | |
| | | | CAG 62 | | ovatus | |
| isobutyrylcarnitine | (9283) S: | −0.05323 | (15356) U: | 0.043612 | (4933) S: | −0.03785 |
| (C4) | Sutterella | | Unknown | | Eubacterium | |
| | wadsworthensis | | | | rectale | |
| X - 12830 | (4577) S: | 0.048621 | (15081) F: | 0.041164 | (15265) S: | 0.029408 |
| | Coprococcus | | Clostridiales | | Firmicutes | |
| | comes | | unclassified | | bacterium | |
| | | | | | CAG 103 | |
| pyroglutamine * | (5785) S: | −0.02204 | (5851) F: | 0.016558 | (15300) S: | 0.015461 |
| | Phascolarctobacterium | | Veillonellaceae | | Gemmiger | |
| | sp | | | | formicilis | |
| | CAG 266 | | | | | |
| X - 11491 | (6148) F: | 0.097771 | (1845) S: | −0.04456 | (9283) S: | −0.03993 |
| | Peptostrep- | | Bacteroides | | Sutterella | |
| | tococcaceae | | intestinalis | | wadsworthensis | |
| | | | CAG 315 | | | |
| N-palmitoyl- | (6140) S: | −0.05902 | (15154) F: | 0.057518 | (2303) S: | −0.04623 |
| sphingosine | Intestinibacter | | Clostridiales | | Alistipes | |
| (d18:1/16:0) | bartlettii | | unclassified | | finegoldii | |
| alpha- | (1814) S: | 0.032839 | (15390) U: | −0.02523 | (6148) F: | 0.024285 |
| hydroxyisocaproate | Bacteroides | | Unknown | | Peptostrep- | |
| | vulgatus | | | | tococcaceae | |
| X - 21410 | (15132) S: | 0.088736 | (1814) S: | 0.079056 | (15332) S: | 0.058448 |
| | Flavonifractor | | Bacteroides | | Faecalibacterium | |
| | plautii | | vulgatus | | prausnitzii | |
| nonadecanoate | (15073) G: | 0.043398 | (6338) F: | 0.028339 | (14974) U: | 0.027807 |
| (19:0) | Oscillibacter | | Clostridiaceae | | Unknown | |
| X - 11478 | (6148) F: | −0.0575 | (6367) F: | −0.03621 | (9226) S: | −0.03547 |
| | Peptostrep- | | Clostridiaceae | | Akkermansia | |
| | tococcaceae | | | | muciniphila | |
| formiminoglutamate | (8002) S: | 0.063505 | (15332) S: | 0.062189 | (6179) G: | 0.050739 |
| | Streptococcus | | Faecalibacterium | | Clostridium | |
| | thermophilus | | prausnitzii | | | |
| X - 11378 | (5736) S: | 0.029329 | (4540) S: | −0.02698 | (2328) F: | −0.02572 |
| | Acidaminococcus | | Anaerostipes | | Rikenellaceae | |
| | intestini | | hadrus | | | |
| erucate | (5075) S: | 0.08554 | (15154) F: | 0.05548 | (4540) S: | −0.04539 |
| (22:1n9) | Lachnospira | | Clostridiales | | Anaerostipes | |
| | pectinoschiza | | unclassified | | hadrus | |
| 7- | (14921) U: | 0.085909 | (4644) S: | −0.04971 | (4537) S: | −0.04821 |
| methylxanthine | Unknown | | Clostridium sp | | Eubacterium | |
| | | | CAG 62 | | hallii | |
| 3- | (4644) S: | −0.0645 | (14921) U: | 0.05496 | (15154) F: | 0.052571 |
| methylxanthine | Clostridium sp | | Unknown | | Clostridiales | |
| | CAG 62 | | | | unclassified | |
| 7-alpha- | (4951) S: | 0.054502 | (1790) S: | −0.04171 | (14909) S: | 0.040551 |
| hydroxy-3-oxo- | Roseburia | | Odoribacter | | Clostridium | |
| 4-cholestenoate | intestinalis | | splanchnicus | | sp CAG | |
| (7-Hoca) | | | | | 169 | |
| 2- | (6179) G: | 0.075033 | (15332) S: | 0.066471 | (4643) S: | −0.05036 |
| aminoadipate | Clostridium | | Faecalibacterium | | Clostridium | |
| | | | prausnitzii | | sp CAG | |
| | | | | | 167 | |
| N- | (15252) F: | 0.035442 | (4608) S: | −0.03347 | (6939) S: | 0.023519 |
| acetylaspartate | Clostridiales | | Ruminococcus | | Veillonella | |
| (NAA) | unclassified | | torques | | parvula | |
| 3- | (14114) S: | 0.057994 | (15256) F: | 0.054033 | (15154) F: | 0.041405 |
| methyladipate | Subdoligranulum | | Clostridiales | | Clostridiales | |
| | sp CAG | | unclassified | | unclassified | |
| | 314 | | | | | |
| gamma- | (6179) G: | 0.047897 | (4714) S: | −0.03889 | (15326) G: | 0.03737 |
| glutamylleucine | Clostridium | | Clostridium sp | | Faecalibacterium | |
| X - 12101 | (6174) S: | 0.041003 | (4652) S: | 0.034255 | (4953) S: | 0.020797 |
| | Clostridium sp | | Clostridium sp | | Roseburia | |
| | CAG 265 | | CAG 75 | | sp CAG | |
| | | | | | 182 | |
| theobromine | (4532) S: | 0.057608 | (4644) S: | −0.05257 | (4537) S: | −0.0471 |
| | Eubacterium | | Clostridium sp | | Eubacterium | |
| | hallii | | CAG 62 | | hallii | |
| 1- | (4577) S: | 0.086446 | (4581) S: | 0.045221 | (4933) S: | −0.0319 |
| methylhistidine | Coprococcus | | Dorea | | Eubacterium | |
| | comes | | longicatena | | rectale | |
| trimethylamine | (17248) S: | −0.03971 | (4721) S: | 0.02819 | (1934) S: | 0.022967 |
| N-oxide | Bifidobacterium | | Clostridium sp | | Parabacteroides | |
| | longum | | CAG 58 | | distasonis | |
| X - 17654 | (17244) S: | 0.053964 | (4581) S: | 0.043974 | (15342) S: | 0.034623 |
| | Bifidobacterium | | Dorea | | Faecalibacterium | |
| | adolescentis | | longicatena | | prausnitzii | |
| ximenoylcarnitine | (1903) S: | 0.10427 | (15346) G: | 0.036248 | (1786) S: | 0.031769 |
| (C26:1)* | Bacteroides | | Faecalibacterium | | Butyricimonas | |
| | plebeius CAG | | | | synergistica | |
| | 211 | | | | | |
| glycosyl | (5843) S: | −0.03897 | (4705) S: | −0.03595 | (4577) S: | −0.02522 |
| ceramide | Allisonella | | Clostridium sp | | Coprococcus | |
| (d18:2/24:1, | histaminiformans | | CAG 43 | | comes | |
| d18:1/24:2)* | | | | | | |
| tiglylcarnitine | (4121) U: | 0.09362 | (8002) S: | 0.045442 | (4577) S: | 0.04075 |
| (C5:1-DC) | Unknown | | Streptococcus | | Coprococcus | |
| | | | thermophilus | | comes | |
| isovalerylglycine | (15339) S: | −0.1054 | (4940) S: | −0.07971 | (14861) U: | 0.074656 |
| | Faecalibacterium | | Roseburia | | Unknown | |
| | prausnitzii | | inulinivorans | | | |
| glutamate | (5075) S: | 0.037256 | (1949) S: | 0.028971 | (9226) S: | −0.02818 |
| | Lachnospira | | Parabacteroides | | Akkermansia | |
| | pectinoschiza | | merdae | | muciniphila | |
| 7-methylurate | (14921) U: | 0.098308 | (15154) F: | 0.092927 | (4537) S: | −0.05381 |
| | Unknown | | Clostridiales | | Eubacterium | |
| | | | unclassified | | hallii | |
| 2- | (4552) S: | 0.055011 | (4933) S: | −0.05078 | (15286) F: | −0.04322 |
| methylbutyrylcarnitine | Ruminococcus | | Eubacterium | | Ruminococcaceae | |
| (C5) | sp | | rectale | | | |
| X - 13844 | (15154) F: | 0.264017 | (14322) S: | 0.105664 | (1872) S: | −0.04441 |
| | Clostridiales | | Eggerthella sp | | Bacteroides | |
| | unclassified | | CAG 209 | | ovatus | |
| X - 12739 | (6140) S: | −0.05159 | (8002) S: | −0.04703 | (1786) S: | 0.024304 |
| | Intestinibacter | | Streptococcus | | Butyricimonas | |
| | bartlettii | | thermophilus | | synergistica | |
| androstenediol | (15154) F: | −0.06373 | (15315) G: | 0.04232 | (9346) S: | −0.03422 |
| (3alpha, | Clostridiales | | Faecalibacterium | | Azospirillum | |
| 17alpha) | unclassified | | | | sp CAG | |
| monosulfate | | | | | 239 | |
| (2) | | | | | | |
| palmitoylcarnitine | (17237) S: | −0.04386 | (6376) F: | −0.03781 | (4804) S: | −0.03548 |
| (C16) | Bifidobacterium | | Clostridiaceae | | Blautia sp | |
| | pseudocatenulatum | | | | | |
| gamma- | (15238) S: | −0.04886 | (15346) G: | 0.04735 | (4951) S: | 0.045291 |
| glutamyl-2- | Firmicutes | | Faecalibacterium | | Roseburia | |
| aminobutyrate | bacterium | | | | intestinalis | |
| | CAG 170 | | | | | |
| acisoga | (4804) S: | −0.0601 | (4749) S: | 0.050771 | (5792) S: | 0.046751 |
| | Blautia sp | | Clostridium sp | | Phascolarctobacterium | |
| | | | CAG 7 | | sp CAG | |
| | | | | | 207 | |
| 1-(1-enyl- | (4705) S: | −0.0961 | (5843) S: | −0.03567 | (4782) U: | 0.0157 |
| palmitoyl)-2- | Clostridium sp | | Allisonella | | Unknown | |
| oleoyl-GPC | CAG 43 | | histaminiformans | | | |
| (P-16:0/18:1)* | | | | | | |
| catechol | (4537) S: | −0.11047 | (14322) S: | 0.056511 | (15154) F: | 0.045195 |
| sulfate | Eubacterium | | Eggerthella sp | | Clostridiales | |
| | hallii | | CAG 209 | | unclassified | |
| 3- | (2290) F: | −0.03072 | (9391) F: | −0.02095 | (15390) U: | −0.01941 |
| methylcytidine | Rikenellaceae | | Oxalobacteraceae | | Unknown | |
| X - 14939 | (17244) S: | 0.07344 | (15271) S: | −0.034 | (8002) S: | −0.03222 |
| | Bifidobacterium | | Ruthenibacterium | | Streptococcus | |
| | adolescentis | | lactatiformans | | thermophilus | |
| pregnenetriol | (8002) S: | −0.04437 | (4940) S: | 0.039132 | (6328) S: | −0.02765 |
| disulfate* | Streptococcus | | Roseburia | | Clostridium | |
| | thermophilus | | inulinivorans | | sp CAG | |
| | | | | | 492 | |
| 1-(1-enyl- | (4577) S: | 0.054186 | (6148) F: | 0.039898 | (17244) S: | −0.03794 |
| stearoyl)-GPE | Coprococcus | | Peptostrep- | | Bifidobacterium | |
| (P-18:0)* | comes | | tococcaceae | | adolescentis | |
| carnitine | (2303) S: | −0.05057 | (4575) S: | 0.042866 | (4933) S: | −0.04003 |
| | Alistipes | | Dorea | | Eubacterium | |
| | finegoldii | | formicigenerans | | rectale | |
| X - 11261 | (4816) S: | −0.03889 | (15216) F: | −0.03588 | (6962) S: | 0.032803 |
| | Blautia sp | | Clostridiales | | Megamonas | |
| | | | unclassified | | funiformis | |
| gamma- | (5082) S: | −0.08064 | (8069) S: | −0.03854 | (14899) U: | 0.035089 |
| glutamylcitrulline* | Eubacterium | | Streptococcus | | Unknown | |
| | eligens | | parasanguinis | | | |
| N-acetyl- | (4960) G: | 0.038558 | (6148) F: | −0.03847 | (4714) S: | 0.028079 |
| isoputreanine* | Eubacterium | | Peptostrep- | | Clostridium sp | |
| | | | tococcaceae | | | |
| 5alpha- | (14252) U: | 0.044691 | (15089) S: | −0.0257 | (4644) S: | 0.024759 |
| pregnan- | Unknown | | Firmicutes | | Clostridium | |
| 3beta,20alpha- | | | bacterium | | sp CAG | |
| diol | | | CAG 83 | | 62 | |
| monosulfate | | | | | | |
| (2) | | | | | | |
| o-cresol sulfate | (15154) F: | 0.11338 | (14993) S: | 0.054371 | (14992) G: | 0.048313 |
| | Clostridiales | | Butyricicoccus | | Butyricicoccus | |
| | unclassified | | sp | | | |
| phenol | (6148) F: | −0.0527 | (4758) S: | 0.036881 | (14861) U: | −0.02897 |
| glucuronide | Peptostrep- | | Clostridium | | Unknown | |
| | tococcaceae | | bolteae | | | |
| leucine | (4714) S: | −0.06304 | (6179) G: | 0.042849 | (15326) G: | −0.03459 |
| | Clostridium sp | | Clostridium | | Faecalibacterium | |
| X - 24544 | (4940) S: | 0.045137 | (8002) S: | −0.04184 | (4750) G: | 0.039685 |
| | Roseburia | | Streptococcus | | Clostridium | |
| | inulinivorans | | thermophilus | | | |
| deoxycholate | (4705) S: | 0.2139 | (14824) F: | 0.084854 | (4749) S: | 0.05729 |
| | Clostridium sp | | Eggerthellaceae | | Clostridium | |
| | CAG 43 | | | | sp CAG | |
| | | | | | 7 | |
| 2-methylserine | (4882) S: | 0.107518 | (14909) S: | −0.09433 | (4933) S: | −0.08823 |
| | Roseburia sp | | Clostridium sp | | Eubacterium | |
| | CAG 100 | | CAG 169 | | rectale | |
| N-stearoyl- | (14253) U: | −0.03739 | (2303) S: | −0.02878 | (8002) S: | 0.024406 |
| sphingosine | Unknown | | Alistipes | | Streptococcus | |
| (d18:1/18:0)* | | | finegoldii | | thermophilus | |
| 2- | (15346) G: | 0.037708 | (4571) S: | 0.027395 | (15238) S: | −0.02494 |
| aminobutyrate | Faecalibacterium | | Dorea sp CAG | | Firmicutes | |
| | | | 105 | | bacterium | |
| | | | | | CAG 170 | |
| imidazole | (15120) S: | −0.03446 | (8076) S: | 0.033378 | (4575) S: | 0.027709 |
| propionate | Firmicutes | | Streptococcus | | Dorea | |
| | bacterium | | parasanguinis | | formicigenerans | |
| | CAG 114 | | | | | |
| sphingomyelin | (15154) F: | 0.044792 | (4670) S: | 0.035114 | (4704) F: | −0.02605 |
| (d18:1/22:1, | Clostridiales | | Coprococcus | | Clostridiaceae | |
| d18:2/22:0, | unclassified | | catus | | | |
| d16:1/24:1)* | | | | | | |
| X - 16944 | (4882) S: | −0.05443 | (4706) F: | −0.02469 | (6340) S: | −0.02445 |
| | Roseburia sp | | Clostridiaceae | | Clostridium | |
| | CAG 100 | | | | sp CAG | |
| | | | | | 269 | |
| X - 24947 | (4940) S: | 0.055102 | (15233) G: | −0.04833 | (4816) S: | −0.03889 |
| | Roseburia | | Firmicutes | | Blautia sp | |
| | inulinivorans | | unclassified | | | |
| indole-3- | (14899) U: | 0.074191 | (14306) S: | 0.022859 | (3996) S: | 0.021827 |
| carboxylic acid | Unknown | | Clostridium sp | | Firmicutes | |
| | | | CAG 138 | | bacterium | |
| | | | | | CAG 145 | |
| perfluorooctanesulfonic | (2303) S: | −0.0692 | (4581) S: | 0.051697 | (4711) F: | −0.04587 |
| acid | Alistipes | | Dorea | | Clostridiaceae | |
| (PFOS) | finegoldii | | longicatena | | | |
| 4- | (14816) F: | 0.090167 | (4705) S: | −0.0882 | (15154) F: | 0.054538 |
| imidazoleacetate | Eggerthellaceae | | Clostridium sp | | Clostridiales | |
| | | | CAG 43 | | unclassified | |
| androstenediol | (4940) S: | 0.042587 | (15326) G: | 0.041594 | (8002) S: | −0.03498 |
| (3alpha,17alpha) | Roseburia | | Faecalibacterium | | Streptococcus | |
| monosulfate | inulinivorans | | | | thermophilus | |
| (3) | | | | | | |
| X - 11444 | (4581) S: | 0.057246 | (5090) S: | −0.03361 | (8007) S: | −0.03187 |
| | Dorea | | Clostridiales | | Streptococcus | |
| | longicatena | | bacterium | | salivarius | |
| | | | KLE1615 | | | |
| N- | (4652) S: | 0.094585 | (15132) S: | −0.09258 | (4957) F: | 0.044323 |
| methyltaurine | Clostridium sp | | Flavonifractor | | Eubacteriaceae | |
| | CAG 75 | | plautii | | | |
| adipoylcarnitine | (15318) S: | 0.076462 | (4552) S: | 0.037269 | (15451) G: | −0.03198 |
| (C6-DC) | Faecalibacterium | | Ruminococcus | | Bilophila | |
| | prausnitzii | | sp | | | |
| X - 18922 | (14861) U: | −0.05743 | (4936) S: | 0.039004 | (14853) S: | −0.03691 |
| | Unknown | | Roseburia | | Clostridium | |
| | | | hominis | | leptum | |
| dehydroisoand | (4940) S: | 0.047967 | (15317) S: | 0.03251 | (4750) G: | 0.030966 |
| rosterone | Roseburia | | Faecalibacterium | | Clostridium | |
| sulfate (DHEA-S) | inulinivorans | | sp CAG 82 | | | |
| perfluorooctanoate | (17256) S: | −0.04541 | (4871) S: | 0.037434 | (5087) S: | 0.033656 |
| (PFOA) | Bifidobacterium | | Ruminococcus | | Eubacterium | |
| | bifidum | | sp | | sp CAG | |
| | | | | | 86 | |
| pregn steroid | (15317) S: | 0.069611 | (9346) S: | −0.0507 | (8002) S: | −0.04222 |
| monosulfate | Faecalibacterium | | Azospirillum | | Streptococcus | |
| C21H34O5S* | sp CAG 82 | | sp CAG 239 | | thermophilus | |
| X - 12798 | (4814) S: | −0.05657 | (4960) G: | −0.03758 | (14921) U: | 0.03569 |
| | Blautia | | Eubacterium | | Unknown | |
| | obeum | | | | | |
| gamma- | (14470) G: | 0.022843 | (4871) S: | 0.022047 | (4553) S: | 0.021627 |
| glutamylglutamate | Collinsella | | Ruminococcus | | Clostridium sp | |
| | | | sp | | | |
| X - 13431 | (4581) S: | 0.073345 | (6750) S: | 0.041298 | (4577) S: | 0.039097 |
| | Dorea | | Clostridium sp | | Coprococcus | |
| | longicatena | | | | comes | |
| caffeic acid | (15154) F: | 0.068272 | (4961) G: | 0.040585 | (4844) S: | −0.02968 |
| sulfate | Clostridiales | | Eubacterium | | Blautia | |
| | unclassified | | | | obeum | |
| 4- | (6308) G: | 0.063085 | (3964) U: | 0.032636 | (4767) U: | −0.03257 |
| hydroxychlorothalonil | Clostridium | | Unknown | | Unknown | |
| X - 17685 | (15154) F: | 0.097833 | (17244) S: | −0.04853 | (15028) G: | −0.04273 |
| | Clostridiales | | Bifidobacterium | | Firmicutes | |
| | unclassified | | adolescentis | | unclassified | |
| thyroxine | (3988) F: | −0.07384 | (15385) U: | −0.04022 | (4721) S: | −0.02471 |
| | Firmicutes | | Unknown | | Clostridium | |
| | unclassified | | | | sp CAG | |
| | | | | | 58 | |
| sphingomyelin | (4540) S: | 0.060424 | (4705) S: | −0.04405 | (6754) S: | 0.02946 |
| (d18:2/24:1, | Anaerostipes | | Clostridium sp | | Clostridium sp | |
| d18:1/24:2)* | hadrus | | CAG 43 | | | |
| Fibrinopeptide | (17241) S: | −0.0508 | (4342) U: | −0.05029 | (9391) F: | −0.04086 |
| A (3-16)** | Bifidobacterium | | Unknown | | Oxalobacteraceae | |
| | catenulatum | | | | | |
| pregnanediol- | (14252) U: | 0.042367 | (15216) F: | 0.034203 | (3957) F: | 0.031372 |
| 3-glucuronide | Unknown | | Clostridiales | | Lachnospiraceae | |
| | | | unclassified | | | |
| N- | (4828) S: | 0.048588 | (15078) S: | −0.0419 | (15265) S: | −0.02874 |
| acetylarginine | Blautia sp | | Oscillibacter | | Firmicutes | |
| | | | sp | | bacterium | |
| | | | | | CAG 103 | |
| pregnen-diol | (8002) S: | −0.04271 | (15317) S: | 0.034709 | (4779) S: | 0.033699 |
| disulfate | Streptococcus | | Faecalibacterium | | Clostridium sp | |
| C21H34O8S2* | thermophilus | | sp CAG 82 | | | |
| 1-oleoyl-2- | (15369) S: | 0.030771 | (4749) S: | −0.02947 | (5076) S: | 0.029252 |
| docosahexaenoyl- | Faecalibacterium | | Clostridium sp | | Eubacterium | |
| GPC | sp CAG 74 | | CAG 7 | | sp CAG | |
| (18:1/22:6)* | | | | | 252 | |
| 3-(4- | (15332) S: | 0.047177 | (15126) S: | −0.04031 | (4425) S: | 0.032587 |
| hydroxyphenyl)lactate | Faecalibacterium | | Intestinimonas | | Ruminococcus | |
| | prausnitzii | | butyriciproducens | | sp CAG | |
| | | | | | 254 | |
| N-acetylglycine | (6754) S: | 0.040646 | (4705) S: | −0.04062 | (15081) F: | −0.03903 |
| | Clostridium sp | | Clostridium sp | | Clostridiales | |
| | | | CAG 43 | | unclassified | |
| propionylglycine | (17241) S: | −0.04787 | (4753) F: | −0.03739 | (4121) U: | 0.034891 |
| | Bifidobacterium | | Lachnospiraceae | | Unknown | |
| | catenulatum | | | | | |
| taurine | (6179) G: | 0.041423 | (1786) S: | 0.03273 | (4537) S: | 0.020686 |
| | Clostridium | | Butyricimonas | | Eubacterium | |
| | | | synergistica | | hallii | |
| glycine | (9226) S: | −0.07967 | (15049) F: | 0.044421 | (4936) S: | 0.041638 |
| conjugate of | Akkermansia | | Clostridiales | | Roseburia | |
| C10H14O2 (1)* | muciniphila | | unclassified | | hominis | |
| sphingomyelin | (4714) S: | −0.03796 | (15154) F: | 0.036928 | (4670) S: | 0.034768 |
| (d18:1/21:0, | Clostridium sp | | Clostridiales | | Coprococcus | |
| d17:1/22:0, | | | unclassified | | catus | |
| d16:1/23:0)* | | | | | | |
| acetylcarnitine | (4940) S: | 0.039533 | (15322) S: | 0.020313 | (4933) S: | −0.01983 |
| (C2) | Roseburia | | Faecalibacterium | | Eubacterium | |
| | inulinivorans | | prausnitzii | | rectale | |
| X - 18899 | (15271) S: | −0.04086 | (14991) F: | 0.026288 | (9391) F: | 0.016897 |
| | Ruthenibacterium | | Clostridiaceae | | Oxalobacteraceae | |
| | lactatiformans | | | | | |
| X - 12906 | (4810) S: | 0.08805 | (4940) S: | −0.05919 | (4705) S: | −0.05477 |
| | Blautia sp | | Roseburia | | Clostridium | |
| | CAG 237 | | inulinivorans | | sp CAG | |
| | | | | | 43 | |
| 3-sulfo-L- | (5076) S: | −0.06914 | (1872) S: | 0.043914 | (1949) S: | 0.042877 |
| alanine | Eubacterium | | Bacteroides | | Parabacteroides | |
| | sp CAG 252 | | ovatus | | merdae | |
| biliverdin | (4842) G: | −0.03558 | (4582) S: | −0.03161 | (4571) S: | 0.030959 |
| | Blautia | | Dorea | | Dorea sp | |
| | | | longicatena | | CAG 105 | |
| 1-linoleoyl- | (5843) S: | −0.05184 | (17248) S: | 0.032153 | (4705) S: | −0.02669 |
| GPA (18:2)* | Allisonella | | Bifidobacterium | | Clostridium | |
| | histaminiformans | | longum | | sp CAG | |
| | | | | | 43 | |
| 3-hydroxy-2- | (14823) F: | 0.057813 | (4552) S: | 0.039847 | (1957) S: | 0.035421 |
| ethylpropionate | Eggerthellaceae | | Ruminococcus | | Bacteroides | |
| | | | sp | | sp CAG | |
| | | | | | 144 | |
| carotene diol | (4705) S: | −0.03301 | (14980) F: | −0.02632 | (4782) U: | 0.025581 |
| (3) | Clostridium sp | | Clostridiaceae | | Unknown | |
| | CAG 43 | | | | | |
| X - 17325 | (14322) S: | 0.061078 | (4537) S: | −0.03802 | (14844) S: | 0.025173 |
| | Eggerthella sp | | Eubacterium | | Firmicutes | |
| | CAG 209 | | hallii | | bacterium | |
| | | | | | CAG 94 | |
| docosahexaenoate | (4905) F: | 0.036171 | (17256) S: | −0.01939 | (1934) S: | 0.018516 |
| (DHA; | Clostridiaceae | | Bifidobacterium | | Parabacteroides | |
| 22:6n3) | | | bifidum | | distasonis | |
| N6,N6,N6- | (6750) S: | 0.041594 | (4828) S: | 0.038495 | (15350) U: | 0.021287 |
| trimethyllysine | Clostridium sp | | Blautia sp | | Unknown | |
| deoxycarnitine | (4575) S: | 0.049704 | (4933) S: | −0.03669 | (4581) S: | 0.031065 |
| | Dorea | | Eubacterium | | Dorea | |
| | formicigenerans | | rectale | | longicatena | |
| 2,3-dihydroxy- | (15236) G: | −0.03061 | (4957) F: | 0.030057 | (4644) S: | −0.02449 |
| 5-methylthio- | Firmicutes | | Eubacteriaceae | | Clostridium | |
| 4-pentenoate | unclassified | | | | sp CAG | |
| (DMTPA)* | | | | | 62 | |
| arabonate/xylonate | (4540) S: | 0.035354 | (1934) S: | 0.029404 | (4648) G: | 0.027639 |
| | Anaerostipes | | Parabacteroides | | Roseburia | |
| | hadrus | | distasonis | | | |
| X - 11852 | (6179) G: | 0.081815 | (4810) S: | 0.054093 | (15260) G: | 0.018769 |
| | Clostridium | | Blautia sp | | Firmicutes | |
| | | | CAG 237 | | unclassified | |
| urea | (4577) S: | 0.079002 | (4121) U: | 0.074149 | (4933) S: | −0.06803 |
| | Coprococcus | | Unknown | | Eubacterium | |
| | comes | | | | rectale | |
| indoleacetylglutamine | (3926) U: | 0.076706 | (13983) U: | 0.035244 | (6754) S: | −0.03479 |
| | Unknown | | Unknown | | Clostridium sp | |
| vanillylmandelate | (4608) S: | −0.06458 | (1872) S: | −0.03822 | (5190) S: | 0.027211 |
| (VMA) | Ruminococcus | | Bacteroides | | Firmicutes | |
| | torques | | ovatus | | bacterium | |
| | | | | | CAG 102 | |
| X - 13255 | (15073) G: | 0.071721 | (15295) G: | 0.063039 | (14322) S: | 0.061006 |
| | Oscillibacter | | Gemmiger | | Eggerthella | |
| | | | | | sp CAG | |
| | | | | | 209 | |
| androstenediol | (4940) S: | 0.051222 | (3964) U: | −0.03788 | (15120) S: | −0.02846 |
| (3beta,17beta) | Roseburia | | Unknown | | Firmicutes | |
| disulfate (1) | inulinivorans | | | | bacterium | |
| | | | | | CAG 114 | |
| valine | (4577) S: | 0.042291 | (4714) S: | −0.03571 | (15339) S: | −0.0347 |
| | Coprococcus | | Clostridium sp | | Faecalibacterium | |
| | comes | | | | prausnitzii | |
| X - 11485 | (1812) S: | 0.095402 | (4953) S: | 0.094858 | (15452) S: | 0.035684 |
| | Bacteroides | | Roseburia sp | | Bilophila | |
| | massiliensis | | CAG 182 | | sp 4 1 30 | |
| X - 24757 | (14322) S: | 0.082937 | (14844) S: | 0.02957 | (8002) S: | 0.022257 |
| | Eggerthella sp | | Firmicutes | | Streptococcus | |
| | CAG 209 | | bacterium | | thermophilus | |
| | | | CAG 94 | | | |
| chenodeoxycholate | (6148) F: | 0.081226 | (2328) F: | −0.05036 | (4914) S: | −0.03421 |
| | Peptostrep- | | Rikenellaceae | | Clostridium sp | |
| | tococcaceae | | | | | |
| 17- | (6174) S: | 0.037474 | (4608) S: | 0.022117 | (6750) S: | 0.021955 |
| methylstearate | Clostridium sp | | Ruminococcus | | Clostridium sp | |
| | CAG 265 | | torques | | | |
| 3- | (4804) S: | −0.07983 | (4940) S: | 0.063162 | (14823) F: | 0.045146 |
| hydroxybutyryl | Blautia sp | | Roseburia | | Eggerthellaceae | |
| carnitine (1) | | | inulinivorans | | | |
| sphingomyelin | (14909) S: | −0.03771 | (4191) S: | 0.019822 | (4705) S: | −0.01832 |
| (d18:2/24:2)* | Clostridium sp | | Eubacterium | | Clostridium | |
| | CAG 169 | | sp CAG 115 | | sp CAG | |
| | | | | | 43 | |
| 5alpha- | (15326) G: | 0.043048 | (4940) S: | 0.042472 | (4198) S: | −0.02814 |
| androstan- | Faecalibacterium | | Roseburia | | Eubacterium | |
| 3beta,17beta- | | | inulinivorans | | siraeum | |
| diol | | | | | | |
| monosulfate | | | | | | |
| (2) | | | | | | |
| stearoyl | (4826) S: | 0.045456 | (4959) S: | 0.038113 | (4670) S: | 0.035029 |
| sphingomyelin | Blautia sp | | Eubacterium | | Coprococcus | |
| (d18:1/18:0) | | | ramulus | | catus | |
| 2- | (8601) S: | −0.05442 | (5068) S: | −0.03666 | (4931) G: | −0.02636 |
| linoleoylglycerol | Candidatus | | Bacteroides | | Lachnospiraceae | |
| (18:2) | Gastranaerophilales | | pectinophilus | | unclassified | |
| | bacterium | | CAG 437 | | | |
| | HUM 10 | | | | | |
| xanthurenate | (8002) S: | 0.121598 | (6140) S: | 0.064645 | (15323) S: | −0.04045 |
| | Streptococcus | | Intestinibacter | | Faecalibacterium | |
| | thermophilus | | bartlettii | | prausnitzii | |
| X - 12411 | (9226) S: | −0.03831 | (6148) F: | 0.022854 | (4577) S: | 0.022152 |
| | Akkermansia | | Peptostrep- | | Coprococcus | |
| | muciniphila | | tococcaceae | | comes | |
| 5-oxoproline | (1786) S: | 0.080071 | (4553) S: | 0.023267 | (5087) S: | 0.019376 |
| | Butyricimonas | | Clostridium sp | | Eubacterium | |
| | synergistica | | | | sp CAG | |
| | | | | | 86 | |
| 1-(1-enyl- | (14921) U: | 0.060526 | (6148) F: | 0.017928 | (14861) U: | 0.016349 |
| palmitoyl)-GPC | Unknown | | Peptostrep- | | Unknown | |
| (P-16:0)* | | | tococcaceae | | | |
| N- | (15154) F: | 0.047969 | (15272) F: | −0.03431 | (4933) S: | −0.02566 |
| acetylglutamate | Clostridiales | | Ruminococcaceae | | Eubacterium | |
| | unclassified | | | | rectale | |
| tetradecanedioate | (1957) S: | 0.075198 | (4874) S: | 0.065262 | (4575) S: | 0.056747 |
| | Bacteroides | | Fusicatenibacter | | Dorea | |
| | sp CAG 144 | | saccharivorans | | formicigenerans | |
| glutarylcarnitine | (9226) S: | −0.02149 | (4564) S: | 0.021473 | (4121) U: | 0.021392 |
| (C5-DC) | Akkermansia | | Ruminococcus | | Unknown | |
| | muciniphila | | torques | | | |
| X - 24337 | (4581) S: | 0.0651 | (2318) S: | −0.04373 | (5082) S: | −0.03891 |
| | Dorea | | Alistipes | | Eubacterium | |
| | longicatena | | putredinis | | eligens | |
| gamma- | (15078) S: | −0.04589 | (6179) G: | 0.038598 | (15326) G: | −0.03007 |
| glutamylisoleucine* | Oscillibacter | | Clostridium | | Faecalibacterium | |
| | sp | | | | | |
| 1-(1-enyl- | (4577) S: | 0.109115 | (14909) S: | 0.052957 | (5792) S: | 0.038317 |
| palmitoyl)-2- | Coprococcus | | Clostridium sp | | Phascolarctobacterium | |
| arachidonoyl- | comes | | CAG 169 | | sp CAG | |
| GPC (P- | | | | | 207 | |
| 16:0/20:4)* | | | | | | |
| 1-(1-enyl- | (4577) S: | 0.083702 | (4960) G: | −0.03791 | (4712) F: | −0.03677 |
| stearoyl)-2- | Coprococcus | | Eubacterium | | Clostridiaceae | |
| oleoyl-GPE | comes | | | | | |
| (P-18:0/18:1) | | | | | | |
| 1-(1-enyl- | (6148) F: | 0.038014 | (4577) S: | 0.035976 | (1862) S: | 0.025325 |
| palmitoyl)-GPE | Peptostrep- | | Coprococcus | | Bacteroides | |
| (P-16:0)* | tococcaceae | | comes | | finegoldii | |
| epiandrosterone | (15315) G: | 0.038067 | (4303) S: | 0.029738 | (3964) U: | −0.02883 |
| sulfate | Faecalibacterium | | Clostridium sp | | Unknown | |
| | | | CAG 217 | | | |
| 2- | (4644) S: | 0.048154 | (5083) G: | 0.034863 | (4547) S: | 0.034632 |
| acetamidophenol | Clostridium sp | | Eubacterium | | Anaerostipes | |
| sulfate | CAG 62 | | | | hadrus | |
| 1-myristoyl-2- | (15326) G: | −0.02009 | (4933) S: | 0.017622 | (15216) F: | −0.01423 |
| arachidonoyl- | Faecalibacterium | | Eubacterium | | Clostridiales | |
| GPC | | | rectale | | unclassified | |
| (14:0/20:4)* | | | | | | |
| N,N,N- | (4581) S: | 0.036467 | (5082) S: | −0.03317 | (4820) S: | −0.0319 |
| trimethyl- | Dorea | | Eubacterium | | Blautia sp | |
| alanylproline | longicatena | | eligens | | | |
| betaine | | | | | | |
| (TMAP) | | | | | | |
| X - 13684 | (1815) S: | −0.0851 | (15106) S: | −0.02385 | (4648) G: | −0.01711 |
| | Bacteroides | | Firmicutes | | Roseburia | |
| | dorei | | bacterium | | | |
| | | | CAG 176 | | | |
| X - 24748 | (5075) S: | 0.024809 | (4933) S: | 0.020641 | (14991) F: | 0.018936 |
| | Lachnospira | | Eubacterium | | Clostridiaceae | |
| | pectinoschiza | | rectale | | | |
| malate | (17249) S: | −0.06347 | (4655) S: | 0.021372 | (15467) S: | 0.018214 |
| | Bifidobacterium | | Clostridium sp | | Desulfovibrio | |
| | longum | | CAG 277 | | piger | |
| isovalerylcarnitine | (6179) G: | 0.048503 | (4581) S: | 0.046117 | (7985) S: | 0.039503 |
| (C5) | Clostridium | | Dorea | | Lactococcus | |
| | | | longicatena | | lactis | |
| 2- | (14924) S: | −0.05728 | (15073) G: | 0.054514 | (2303) S: | −0.04394 |
| hydroxynervonate* | Firmicutes | | Oscillibacter | | Alistipes | |
| | bacterium | | | | finegoldii | |
| | CAG 137 | | | | | |
| X - 11858 | (15078) S: | −0.02592 | (15132) S: | −0.01619 | (15124) F: | −0.01299 |
| | Oscillibacter | | Flavonifractor | | Clostridiales | |
| | sp | | plautii | | unclassified | |
| 3- | (15154) F: | 0.096561 | (2326) S: | −0.03556 | (4537) S: | −0.03231 |
| hydroxyhippurate | Clostridiales | | Faecalibacterium | | Eubacterium | |
| sulfate | unclassified | | prausnitzii | | hallii | |
| lactosyl-N- | (7985) S: | −0.04284 | (4705) S: | −0.04131 | (8002) S: | −0.0391 |
| nervonoyl- | Lactococcus | | Clostridium sp | | Streptococcus | |
| sphingosine | lactis | | CAG 43 | | thermophilus | |
| (d18:1/24:1)* | | | | | | |
| 1-(1-enyl- | (4577) S: | 0.040681 | (5190) S: | 0.031594 | (2311) F: | 0.027897 |
| palmitoyl)-2- | Coprococcus | | Firmicutes | | Rikenellaceae | |
| oleoyl-GPE | comes | | bacterium | | | |
| (P-16:0/18:1)* | | | CAG 102 | | | |
| X - 18886 | (4940) S: | 0.1102 | (14993) S: | 0.030132 | (14823) F: | 0.028101 |
| | Roseburia | | Butyricicoccus | | Eggerthellaceae | |
| | inulinivorans | | sp | | | |
| Fibrinopeptide | (4342) U: | −0.04477 | (9391) F: | −0.03691 | (4553) S: | −0.03651 |
| B (1-13)** | Unknown | | Oxalobacteraceae | | Clostridium sp | |
| taurochenodeoxycholic | (4749) S: | −0.03801 | (4705) S: | −0.01807 | (4367) S: | −0.01552 |
| acid 3- | Clostridium sp | | Clostridium sp | | Ruminococcus | |
| sulfate | CAG 7 | | CAG 43 | | sp CAG | |
| | | | | | 177 | |
| DSGEGDFXAEGGGVR* | (17241) S: | −0.0563 | (4342) U: | −0.05006 | (1786) S: | −0.04787 |
| | Bifidobacterium | | Unknown | | Butyricimonas | |
| | catenulatum | | | | synergistica | |
| tauroursodeoxycholate | (4552) S: | −0.06347 | (14844) S: | 0.034069 | (15154) F: | 0.033536 |
| | Ruminococcus | | Firmicutes | | Clostridiales | |
| | sp | | bacterium | | unclassified | |
| | | | CAG 94 | | | |
| X - 13723 | (15154) F: | 0.099408 | (14322) S: | 0.064691 | (1872) S: | −0.03785 |
| | Clostridiales | | Eggerthella sp | | Bacteroides | |
| | unclassified | | CAG 209 | | ovatus | |
| 1-stearoyl-2- | (15229) F: | −0.03036 | (4448) G: | 0.029146 | (4804) S: | −0.02635 |
| docosahexaenoyl-GPE | Clostridiales | | Eubacterium | | Blautia sp | |
| (18:0/22:6)* | unclassified | | | | | |
| 14-HDoHE/17- | (6179) G: | 0.09176 | (4191) S: | 0.032071 | (15081) F: | 0.027606 |
| HDoHE | Clostridium | | Eubacterium | | Clostridiales | |
| | | | sp CAG 115 | | unclassified | |
| 1- | (15216) F: | −0.01615 | (5090) S: | −0.01475 | (4828) S: | 0.009504 |
| linolenoylglycerol | Clostridiales | | Clostridiales | | Blautia sp | |
| (18:3) | unclassified | | bacterium | | | |
| | | | KLE1615 | | | |
| X - 11299 | (1836) S: | −0.0339 | (14797) G: | 0.031022 | (5785) S: | 0.028369 |
| | Bacteroides | | Adlercreutzia | | Phascolarctobacterium | |
| | uniformis | | | | sp CAG | |
| | | | | | 266 | |
| X - 21285 | (4940) S: | 0.084041 | (15233) G: | −0.04693 | (4581) S: | 0.04181 |
| | Roseburia | | Firmicutes | | Dorea | |
| | inulinivorans | | unclassified | | longicatena | |
| Fibrinopeptide | (17241) S: | −0.04287 | (4342) U: | −0.04129 | (9391) F: | −0.03389 |
| A (5-16)* | Bifidobacterium | | Unknown | | Oxalobacteraceae | |
| | catenulatum | | | | | |
| X - 21661 | (15078) S: | −0.01988 | (15124) F: | −0.01812 | (4582) S: | −0.01351 |
| | Oscillibacter | | Clostridiales | | Dorea | |
| | sp | | unclassified | | longicatena | |
| dodecenedioate | (6465) S: | 0.054859 | (4964) F: | 0.035591 | (4839) G: | −0.0334 |
| (C12:1-DC)* | Mycoplasma | | Eubacteriaceae | | Blautia | |
| | sp CAG 611 | | | | | |
| 3-methyl-2- | (14909) S: | 0.03462 | (4951) S: | 0.027326 | (15089) S: | −0.02103 |
| oxovalerate | Clostridium sp | | Roseburia | | Firmicutes | |
| | CAG 169 | | intestinalis | | bacterium | |
| | | | | | CAG 83 | |
| X - 11847 | (15078) S: | −0.02382 | (4811) S: | −0.02231 | (14542) G: | −0.01526 |
| | Oscillibacter | | Blautia | | Collinsella | |
| | sp | | obeum | | | |
| 1-myristoyl-2- | (15233) G: | −0.04287 | (4936) S: | −0.03127 | (8002) S: | 0.01649 |
| palmitoyl-GPC | Firmicutes | | Roseburia | | Streptococcus | |
| (14:0/16:0) | unclassified | | hominis | | thermophilus | |
| 3-aminoisobutyrate | (4130) U: | 0.048514 | (14992) G: | 0.038371 | (3940) U: | 0.02967 |
| | Unknown | | Butyricicoccus | | Unknown | |
| stachydrine | (4960) G: | 0.111027 | (5089) S: | 0.041966 | (4582) S: | −0.03922 |
| | Eubacterium | | Eubacterium | | Dorea | |
| | | | sp CAG 38 | | longicatena | |
| eicosenoate | (14992) G: | 0.03625 | (15154) F: | 0.03301 | (15073) G: | 0.024107 |
| (20:1) | Butyricicoccus | | Clostridiales | | Oscillibacter | |
| | | | unclassified | | | |
| isocitrate | (6754) S: | 0.092134 | (4714) S: | 0.077375 | (4779) S: | −0.04582 |
| | Clostridium sp | | Clostridium sp | | Clostridium sp | |
| X - 21364 | (4750) G: | 0.040436 | (4581) S: | 0.023042 | (4564) S: | 0.021019 |
| | Clostridium | | Dorea | | Ruminococcus | |
| | | | longicatena | | torques | |
| X - 12007 | (4960) G: | −0.03341 | (4782) U: | 0.02307 | (4532) S: | 0.021818 |
| | Eubacterium | | Unknown | | Eubacterium | |
| | | | | | hallii | |
| N1-Methyl-2- | (4577) S: | 0.067729 | (14999) U: | 0.040532 | (14861) U: | 0.033075 |
| pyridone-5- | Coprococcus | | Unknown | | Unknown | |
| carboxamide | comes | | | | | |
| X - 21659 | (1812) S: | 0.067379 | (4577) S: | 0.053012 | (4964) F: | 0.050115 |
| | Bacteroides | | Coprococcus | | Eubacteriaceae | |
| | massiliensis | | comes | | | |
| gamma- | (4540) S: | −0.03464 | (15124) F: | −0.02828 | (5792) S: | −0.02305 |
| tocopherol/beta- | Anaerostipes | | Clostridiales | | Phascolarctobacterium | |
| tocopherol | hadrus | | unclassified | | sp CAG | |
| | | | | | 207 | |
| X - 12117 | (15326) G: | −0.03722 | (1790) S: | −0.03141 | (4670) S: | −0.02739 |
| | Faecalibacterium | | Odoribacter | | Coprococcus | |
| | | | splanchnicus | | catus | |
| 1- | (15452) S: | 0.03446 | (5190) S: | −0.02729 | (15332) S: | 0.02379 |
| myristoylglycerol | Bilophila sp 4 | | Firmicutes | | Faecalibacterium | |
| (14:0) | 1 30 | | bacterium | | prausnitzii | |
| | | | CAG 102 | | | |
| X - 21845 | (14816) F: | −0.0468 | (4577) S: | 0.038822 | (1812) S: | 0.032944 |
| | Eggerthellaceae | | Coprococcus | | Bacteroides | |
| | | | comes | | massiliensis | |
| N- | (4960) G: | 0.071663 | (4933) S: | −0.03528 | (4871) S: | −0.03173 |
| methylhydroxy | Eubacterium | | Eubacterium | | Ruminococcus | |
| proline** | | | rectale | | sp | |
| stearoylcarnitine | (6750) S: | 0.065403 | (17237) S: | −0.02695 | (4780) G: | −0.02624 |
| (C18) | Clostridium sp | | Bifidobacterium | | Clostridium | |
| | | | pseudocatenulatum | | | |
| X - 24546 | (14991) F: | −0.07482 | (15350) U: | 0.046263 | (4197) G: | −0.03719 |
| | Clostridiaceae | | Unknown | | Ruminiclostridium | |
| 2- | (14921) U: | 0.021881 | (15216) F: | −0.01795 | (1797) S: | 0.017445 |
| hydroxyglutarate | Unknown | | Clostridiales | | Paraprevotella | |
| | | | unclassified | | xylaniphila | |
| X - 23787 | (4940) S: | 0.080894 | (4564) S: | 0.024221 | (15299) G: | 0.021077 |
| | Roseburia | | Ruminococcus | | Gemmiger | |
| | inulinivorans | | torques | | | |
| 4- | (4714) S: | 0.040799 | (14992) G: | 0.037468 | (4940) S: | −0.03035 |
| hydroxyhippurate | Clostridium sp | | Butyricicoccus | | Roseburia | |
| | | | | | inulinivorans | |
| glycylvaline | (1786) S: | 0.056913 | (17241) S: | 0.055379 | (4342) U: | 0.025881 |
| | Butyricimonas | | Bifidobacterium | | Unknown | |
| | synergistica | | catenulatum | | | |
| cerotoylcarnitine | (4828) S: | 0.058453 | (1903) S: | 0.038339 | (4779) S: | 0.03178 |
| (C26)* | Blautia sp | | Bacteroides | | Clostridium sp | |
| | | | plebeius CAG | | | |
| | | | 211 | | | |
| methylsuccinoylcarnitine | (4933) S: | −0.0424 | (9226) S: | 0.020355 | (6173) S: | 0.01984 |
| (1) | Eubacterium | | Akkermansia | | Clostridium | |
| | rectale | | muciniphila | | sp CAG | |
| | | | | | 221 | |
| X - 15492 | (5843) S: | 0.040621 | (14894) S: | −0.03993 | (1815) S: | −0.03886 |
| | Allisonella | | Anaeromassili | | Bacteroides | |
| | histaminiformans | | bacillus sp | | dorei | |
| | | | An250 | | | |
| X - 23585 | (15451) G: | 0.054984 | (15229) F: | 0.049947 | (2311) F: | 0.033186 |
| | Bilophila | | Clostridiales | | Rikenellaceae | |
| | | | unclassified | | | |
| X - 24556 | (1934) S: | 0.048685 | (17244) S: | −0.03436 | (4303) S: | 0.027297 |
| | Parabacteroides | | Bifidobacterium | | Clostridium | |
| | distasonis | | adolescentis | | sp CAG | |
| | | | | | 217 | |
| N1- | (6962) S: | 0.021221 | (15216) F: | −0.01933 | (5043) S: | −0.01892 |
| methyladenosine | Megamonas | | Clostridiales | | Eubacterium | |
| | funiformis | | unclassified | | sp CAG | |
| | | | | | 156 | |
| 1,2,3- | (15154) F: | 0.03668 | (5111) S: | −0.02883 | (14773) F: | −0.02293 |
| benzenetriol | Clostridiales | | Clostridium sp | | Eggerthellaceae | |
| sulfate (2) | unclassified | | CAG 127 | | | |
| 21- | (4882) S: | −0.05051 | (8002) S: | −0.04853 | (6334) F: | −0.04584 |
| hydroxypregnenolone | Roseburia sp | | Streptococcus | | Clostridiaceae | |
| disulfate | CAG 100 | | thermophilus | | | |
| hexanoylglutamine | (2328) F: | 0.054311 | (4940) S: | 0.047796 | (17249) S: | −0.04182 |
| | Rikenellaceae | | Roseburia | | Bifidobacterium | |
| | | | inulinivorans | | longum | |
| X - 17367 | (14322) S: | 0.056771 | (14844) S: | 0.022509 | (15085) F: | 0.015576 |
| | Eggerthella sp | | Firmicutes | | Clostridiales | |
| | CAG 209 | | bacterium | | unclassified | |
| | | | CAG 94 | | | |
| tridecenedioate | (4826) S: | 0.074521 | (14921) U: | 0.053997 | (4714) S: | −0.0487 |
| (C13:1-DC)* | Blautia sp | | Unknown | | Clostridium sp | |
| phytanate | (14974) U: | 0.023498 | (5075) S: | 0.01916 | (4940) S: | 0.017494 |
| | Unknown | | Lachnospira | | Roseburia | |
| | | | pectinoschiza | | inulinivorans | |
| hydroxy- | (1798) S: | −0.0339 | (5803) S: | −0.03266 | (6141) F: | 0.031911 |
| CMPF* | Paraprevotella | | Dialister sp | | Peptostrep- | |
| | clara | | CAG 357 | | tococcaceae | |
| N-palmitoyl- | (4659) S: | 0.032555 | (5062) G: | −0.03093 | (15315) G: | −0.02939 |
| sphinganine | Clostridium sp | | Firmicutes | | Faecalibacterium | |
| (d18:0/16:0) | CAG 122 | | unclassified | | | |
| 4-methyl-2- | (14909) S: | 0.038456 | (4951) S: | 0.025853 | (15390) U: | −0.02486 |
| oxopentanoate | Clostridium sp | | Roseburia | | Unknown | |
| | CAG 169 | | intestinalis | | | |
| cys-gly, | (14020) U: | −0.0333 | (15350) U: | 0.02773 | (15299) G: | 0.02709 |
| oxidized | Unknown | | Unknown | | Gemmiger | |
| glycerate | (4540) S: | 0.054775 | (4714) S: | 0.031525 | (4705) S: | −0.02888 |
| | Anaerostipes | | Clostridium sp | | Clostridium | |
| | hadrus | | | | sp CAG | |
| | | | | | 43 | |
| bradykinin, | (15158) G: | 0.012129 | (5184) U: | 0.011144 | (6140) S: | −0.00955 |
| des-arg(9) | Flavonifractor | | Unknown | | Intestinibacter | |
| | | | | | bartlettii | |
| 15- | (1957) S: | 0.025477 | (14823) F: | 0.021239 | (15132) S: | 0.019465 |
| methylpalmitate | Bacteroides | | Eggerthellaceae | | Flavonifractor | |
| | sp CAG 144 | | | | plautii | |
| X - 11795 | (4608) S: | −0.05172 | (6750) S: | −0.04128 | (17244) S: | 0.037572 |
| | Ruminococcus | | Clostridium sp | | Bifidobacterium | |
| | torques | | | | adolescentis | |
| 16a-hydroxy | (14991) F: | −0.02375 | (4750) G: | 0.021978 | (6334) F: | −0.01932 |
| DHEA 3-sulfate | Clostridiaceae | | Clostridium | | Clostridiaceae | |
| arachidoylcarnitine | (6750) S: | 0.087103 | (1872) S: | −0.06921 | (17249) S: | −0.06082 |
| (C20)* | Clostridium sp | | Bacteroides | | Bifidobacterium | |
| | | | ovatus | | longum | |
| choline | (6179) G: | 0.02188 | (4198) S: | 0.018296 | (14252) U: | 0.015386 |
| | Clostridium | | Eubacterium | | Unknown | |
| | | | siraeum | | | |
| palmitoyl | (4964) F: | 0.054995 | (4834) G: | 0.038795 | (4953) S: | 0.022056 |
| dihydrosphingomyelin | Eubacteriaceae | | Blautia | | Roseburia | |
| (d18:0/16:0)* | | | | | sp CAG | |
| | | | | | 182 | |
| glycosyl-N- | (4961) G: | 0.043081 | (15390) U: | 0.030939 | (4780) G: | 0.022634 |
| behenoyl- | Eubacterium | | Unknown | | Clostridium | |
| sphingadienine | | | | | | |
| (d18:2/22:0)* | | | | | | |
| hydroxy- | (15216) F: | −0.02635 | (15028) G: | −0.01812 | (9226) S: | −0.01758 |
| N6,N6,N6- | Clostridiales | | Firmicutes | | Akkermansia | |
| trimethyllysine * | unclassified | | unclassified | | muciniphila | |
| lysine | (17278) S: | 0.052259 | (15332) S: | 0.045987 | (6179) G: | 0.038203 |
| | Bifidobacterium | | Faecalibacterium | | Clostridium | |
| | animalis | | prausnitzii | | | |
| tyrosine | (4425) S: | 0.056453 | (8002) S: | 0.040884 | (6179) G: | 0.040077 |
| | Ruminococcus | | Streptococcus | | Clostridium | |
| | sp CAG 254 | | thermophilus | | | |
| androsterone | (4940) S: | 0.043759 | (15315) G: | 0.033205 | (3964) U: | −0.02985 |
| sulfate | Roseburia | | Faecalibacterium | | Unknown | |
| | inulinivorans | | | | | |
| glycodeoxycholate | (4705) S: | 0.045146 | (4829) S: | −0.03794 | (4540) S: | −0.03277 |
| sulfate | Clostridium sp | | Blautia sp | | Anaerostipes | |
| | CAG 43 | | | | hadrus | |
| alpha- | (15154) F: | 0.040527 | (1862) S: | 0.035612 | (1934) S: | 0.027055 |
| tocopherol | Clostridiales | | Bacteroides | | Parabacteroides | |
| | unclassified | | finegoldii | | distasonis | |
| 3-(3- | (15154) F: | 0.083726 | (4938) S: | −0.02004 | (2326) S: | −0.01745 |
| hydroxyphenyl)propionate | Clostridiales | | Roseburia sp | | Faecalibacterium | |
| sulfate | unclassified | | | | prausnitzii | |
| linoleate | (15120) S: | −0.02249 | (15073) G: | 0.020876 | (3957) F: | −0.02028 |
| (18:2n6) | Firmicutes | | Oscillibacter | | Lachnospiraceae | |
| | bacterium | | | | | |
| | CAG 114 | | | | | |
| 17alpha- | (15317) S: | 0.055301 | (8002) S: | −0.03468 | (4779) S: | 0.032196 |
| hydroxypregnenolone 3- | Faecalibacterium | | Streptococcus | | Clostridium sp | |
| sulfate | sp CAG 82 | | thermophilus | | | |
| xanthosine | (9712) S: | 0.043563 | (6506) S: | −0.02903 | (4537) S: | −0.02411 |
| | Haemophilus | | Mycoplasma | | Eubacterium | |
| | parainfluenzae | | sp CAG 472 | | hallii | |
| 4- | (8002) S: | 0.075469 | (15090) S: | 0.033012 | (6179) G: | 0.031415 |
| hydroxyphenyl | Streptococcus | | Oscillibacter | | Clostridium | |
| pyruvate | thermophilus | | sp CAG 241 | | | |
| S- | (15132) S: | −0.03076 | (4652) S: | 0.024054 | (1814) S: | −0.01913 |
| methylcysteine | Flavonifractor | | Clostridium sp | | Bacteroides | |
| | plautii | | CAG 75 | | vulgatus | |
| dodecadienoate | (15120) S: | −0.03576 | (6174) S: | 0.012225 | (15073) G: | 0.008508 |
| (12:2)* | Firmicutes | | Clostridium sp | | Oscillibacter | |
| | bacterium | | CAG 265 | | | |
| | CAG 114 | | | | | |
| 1-palmitoyl-2- | (4936) S: | −0.02797 | (15233) G: | −0.02551 | (1790) S: | −0.02175 |
| palmitoleoyl- | Roseburia | | Firmicutes | | Odoribacter | |
| GPC | hominis | | unclassified | | splanchnicus | |
| (16:0/16:1)* | | | | | | |
| 2- | (4582) S: | −0.05742 | (15342) S: | 0.054321 | (9340) F: | −0.03421 |
| arachidonoylglycerol | Dorea | | Faecalibacterium | | Rhodospirillaceae | |
| (20:4) | longicatena | | prausnitzii | | | |
| sphingomyelin | (4714) S: | −0.07138 | (4826) S: | 0.030292 | (6148) F: | 0.027858 |
| (d18:1/25:0, | Clostridium sp | | Blautia sp | | Peptostrep- | |
| d19:0/24:1, | | | | | tococcaceae | |
| d20:1/23:0, | | | | | | |
| d19:1/24:0)* | | | | | | |
| 1-palmitoyl-2- | (1862) S: | 0.03076 | (1786) S: | 0.028072 | (1948) S: | 0.019686 |
| docosahexaenoyl- | Bacteroides | | Butyricimonas | | Parabacteroides | |
| GPC | finegoldii | | synergistica | | johnsonii | |
| (16:0/22:6) | | | | | | |
| Fibrinopeptide | (4342) U: | −0.03571 | (5045) S: | −0.0354 | (17241) S: | −0.03458 |
| A (7-16)* | Unknown | | Eubacterium | | Bifidobacterium | |
| | | | ventriosum | | catenulatum | |
| N6- | (4834) G: | −0.0168 | (15154) F: | −0.01297 | (6750) S: | 0.010781 |
| carbamoylthre- | Blautia | | Clostridiales | | Clostridium sp | |
| onyladenosine | | | unclassified | | | |
| glycohyocholate | (5843) S: | −0.04941 | (15317) S: | 0.048986 | (4705) S: | −0.04526 |
| | Allisonella | | Faecalibacterium | | Clostridium | |
| | histaminiformans | | sp CAG 82 | | sp CAG | |
| | | | | | 43 | |
| N- | (4779) S: | −0.05561 | (6174) S: | 0.037775 | (1790) S: | 0.031518 |
| oleoyltaurine | Clostridium sp | | Clostridium sp | | Odoribacter | |
| | | | CAG 265 | | splanchnicus | |
| X - 11593 | (4644) S: | −0.0252 | (1790) S: | −0.01633 | (8002) S: | 0.013312 |
| | Clostridium sp | | Odoribacter | | Streptococcus | |
| | CAG 62 | | splanchnicus | | thermophilus | |
| phenyllactate | (14424) G: | 0.032886 | (15146) F: | 0.022813 | (2290) F: | −0.02269 |
| (PLA) | Collinsella | | Clostridiales | | Rikenellaceae | |
| | | | unclassified | | | |
| beta- | (5087) S: | 0.026445 | (17256) S: | −0.02453 | (4871) S: | 0.024255 |
| citrylglutamate | Eubacterium | | Bifidobacterium | | Ruminococcus sp | |
| | sp CAG 86 | | bifidum | | | |
| X - 14314 | (14861) U: | 0.018415 | (6179) G: | 0.016682 | (5087) S: | 0.015524 |
| | Unknown | | Clostridium | | Eubacterium | |
| | | | | | sp CAG | |
| | | | | | 86 | |
| creatine | (5785) S: | 0.033942 | (9283) S: | −0.03123 | (15051) F: | −0.01673 |
| | Phascolarctobacterium | | Sutterella | | Clostridiales | |
| | sp | | wadsworthensis | | unclassified | |
| | CAG 266 | | | | | |
| arabitol/xylitol | (4648) G: | 0.040273 | (4532) S: | 0.0356 | (15054) F: | −0.0352 |
| | Roseburia | | Eubacterium | | Clostridiales | |
| | | | hallii | | unclassified | |
| uridine | (4714) S: | 0.051311 | (5045) S: | 0.049398 | (4261) G: | 0.045529 |
| | Clostridium sp | | Eubacterium | | Blautia | |
| | | | ventriosum | | | |
| ectoine | (15019) F: | 0.043657 | (4577) S: | 0.030343 | (15078) S: | −0.01206 |
| | Clostridiales | | Coprococcus | | Oscillibacter sp | |
| | unclassified | | comes | | | |
| X - 17653 | (17244) S: | 0.042806 | (15266) G: | 0.016781 | (17237) S: | −0.01493 |
| | Bifidobacterium | | Firmicutes | | Bifidobacterium | |
| | adolescentis | | unclassified | | pseudocatenulatum | |
| catechol | (14322) S: | 0.070021 | (4537) S: | −0.03782 | (1877) S: | −0.02423 |
| glucuronide | Eggerthella sp | | Eubacterium | | Bacteroides | |
| | CAG 209 | | hallii | | caccae | |
| X - 18887 | (15318) S: | 0.061995 | (15315) G: | 0.042437 | (15132) S: | −0.02926 |
| | Faecalibacterium | | Faecalibacterium | | Flavonifractor | |
| | prausnitzii | | | | plautii | |
| eicosapentaenoylcholine | (9226) S: | 0.139858 | (4782) U: | −0.06037 | (4771) G: | 0.051517 |
| | Akkermansia | | Unknown | | Clostridium | |
| | muciniphila | | | | | |
| oleate/vaccenate | (15073) G: | 0.02365 | (4804) S: | −0.01695 | (13982) U: | 0.015379 |
| (18:1) | Oscillibacter | | Blautia sp | | Unknown | |
| N- | (8007) S: | 0.021658 | (15318) S: | 0.019103 | (4804) S: | −0.0164 |
| acetylneuraminate | Streptococcus | | Faecalibacterium | | Blautia sp | |
| | salivarius | | prausnitzii | | | |
| X - 16576 | (4842) G: | 0.027401 | (1830) S: | 0.020524 | (14507) G: | 0.01622 |
| | Blautia | | Bacteroides | | Collinsella | |
| | | | stercoris | | | |
| X - 21839 | (4953) S: | 0.035652 | (1812) S: | 0.028987 | (15369) S: | 0.024946 |
| | Roseburia sp | | Bacteroides | | Faecalibacterium | |
| | CAG 182 | | massiliensis | | sp | |
| | | | | | CAG 74 | |
| 1-palmitoyl-2- | (15216) F: | −0.05724 | (4422) S: | −0.03299 | (1790) S: | −0.03035 |
| gamma- | Clostridiales | | Ruminococcus | | Odoribacter | |
| linolenoyl-GPC | unclassified | | callidus | | splanchnicus | |
| (16:0/18:3n6)* | | | | | | |
| 2- | (1965) S: | −0.02435 | (5803) S: | 0.017444 | (5089) S: | 0.01367 |
| aminoheptanoate | Bacteroides | | Dialister sp | | Eubacterium | |
| | sp CAG 20 | | CAG 357 | | sp CAG | |
| | | | | | 38 | |
| palmitoyl | (15395) U: | 0.024214 | (4670) S: | 0.018061 | (15154) F: | 0.017628 |
| sphingomyelin | Unknown | | Coprococcus | | Clostridiales | |
| (d18:1/16:0) | | | catus | | unclassified | |
| nervonoylcarnitine | (4828) S: | 0.076953 | (6139) G: | 0.063631 | (15373) F: | −0.04745 |
| (C24:1)* | Blautia sp | | Intestinibacter | | Ruminococcaceae | |
| X - 24812 | (15154) F: | 0.060738 | (15332) S: | 0.046498 | (15322) S: | 0.040435 |
| | Clostridiales | | Faecalibacterium | | Faecalibacterium | |
| | unclassified | | prausnitzii | | prausnitzii | |
| piperine | (4953) S: | 0.04313 | (4577) S: | 0.022051 | (1812) S: | 0.02156 |
| | Roseburia sp | | Coprococcus | | Bacteroides | |
| | CAG 182 | | comes | | massiliensis | |
| chiro-inositol | (4960) G: | 0.08841 | (4961) G: | 0.030103 | (4714) S: | 0.026816 |
| | Eubacterium | | Eubacterium | | Clostridium sp | |
| X - 23974 | (5082) S: | 0.079081 | (4882) S: | 0.042061 | (5045) S: | −0.03954 |
| | Eubacterium | | Roseburia sp | | Eubacterium | |
| | eligens | | CAG 100 | | ventriosum | |
| 3- | (15154) F: | 0.047145 | (15028) G: | −0.02601 | (14773) F: | −0.02444 |
| methoxycatechol | Clostridiales | | Firmicutes | | Eggerthellaceae | |
| sulfate (1) | unclassified | | unclassified | | | |
| N-trimethyl 5- | (1626) S: | −0.03739 | (8002) S: | 0.034481 | (17278) S: | 0.02913 |
| aminovalerate | Prevotella | | Streptococcus | | Bifidobacterium | |
| | copri | | thermophilus | | animalis | |
| glycochenodeoxycholate | (4829) S: | −0.03975 | (4540) S: | −0.0345 | (1867) S: | −0.03082 |
| glucuronide (1) | Blautia sp | | Anaerostipes | | Bacteroides | |
| | | | hadrus | | xylanisolvens | |
| sphingomyelin | (14894) S: | 0.037895 | (5843) S: | −0.02739 | (5082) S: | 0.023923 |
| (d18:1/20:1, | Anaeromassili | | Allisonella | | Eubacterium | |
| d18:2/20:0)* | bacillus sp | | histaminiformans | | eligens | |
| | An250 | | | | | |
| X - 11470 | (6750) S: | 0.042303 | (14894) S: | −0.03208 | (17237) S: | −0.02299 |
| | Clostridium sp | | Anaeromassili | | Bifidobacterium | |
| | | | bacillus sp | | pseudocatenulatum | |
| | | | An250 | | | |
| X - 21353 | (4940) S: | 0.027706 | (14993) S: | 0.016742 | (15271) S: | −0.01657 |
| | Roseburia | | Butyricicoccus | | Ruthenibacterium | |
| | inulinivorans | | sp | | lactatiformans | |
| X - 12472 | (15073) G: | 0.04229 | (14992) G: | 0.036848 | (15089) S: | −0.02617 |
| | Oscillibacter | | Butyricicoccus | | Firmicutes | |
| | | | | | bacterium | |
| | | | | | CAG 83 | |
| X - 12456 | (1782) G: | 0.035023 | (15265) S: | 0.029642 | (4447) S: | 0.025897 |
| | Butyricimonas | | Firmicutes | | Eubacterium | |
| | | | bacterium | | sp CAG | |
| | | | CAG 103 | | 274 | |
| X - 13866 | (17244) S: | −0.0256 | (17256) S: | −0.0203 | (17248) S: | −0.01956 |
| | Bifidobacterium | | Bifidobacterium | | Bifidobacterium | |
| | adolescentis | | bifidum | | longum | |
| vanillactate | (15318) S: | 0.116428 | (4820) S: | −0.10834 | (9283) S: | −0.0534 |
| | Faecalibacterium | | Blautia sp | | Sutterella | |
| | prausnitzii | | | | wadsworthensis | |
| X - 16580 | (15236) G: | −0.04338 | (14974) U: | 0.035757 | (14823) F: | 0.032492 |
| | Firmicutes | | Unknown | | Eggerthellaceae | |
| | unclassified | | | | | |
| X - 24329 | (9226) S: | −0.03553 | (8601) S: | −0.03325 | (15342) S: | 0.030059 |
| | Akkermansia | | Candidatus | | Faecalibacterium | |
| | muciniphila | | Gastranaerophi- | | prausnitzii | |
| | | | lalesbacterium | | | |
| | | | HUM 10 | | | |
| androsterone | (3964) U: | −0.02893 | (4581) S: | 0.026443 | (4540) S: | −0.02591 |
| glucuronide | Unknown | | Dorea | | Anaerostipes | |
| | | | longicatena | | hadrus | |
| hydroxyasparagine** | (15286) F: | −0.01919 | (14894) S: | −0.0183 | (4644) S: | −0.00988 |
| | Ruminococcaceae | | Anaeromassili | | Clostridium | |
| | | | bacillus sp | | sp CAG | |
| | | | An250 | | 62 | |
| X - 23680 | (4816) S: | −0.05875 | (15049) F: | 0.037889 | (4644) S: | −0.02805 |
| | Blautia sp | | Clostridiales | | Clostridium | |
| | | | unclassified | | sp CAG | |
| | | | | | 62 | |
| 1- | (4871) S: | 0.035893 | (15216) F: | −0.03174 | (1785) S: | 0.028502 |
| oleoylglycerol | Ruminococcus | | Clostridiales | | Butyricimonas | |
| (18:1) | sp | | unclassified | | sp | |
| | | | | | An62 | |
| 1-(1-enyl- | (5843) S: | −0.04398 | (4782) U: | 0.01852 | (4540) S: | 0.017006 |
| palmitoyl)-2- | Allisonella | | Unknown | | Anaerostipes | |
| palmitoleoyl- | histaminiformans | | | | hadrus | |
| GPC | | | | | | |
| (P-16:0/16:1)* | | | | | | |
| heneicosapentaenoate | (1862) S: | 0.028959 | (1830) S: | 0.028546 | (1934) S: | 0.027349 |
| (21:5n3) | Bacteroides | | Bacteroides | | Parabacteroides | |
| | finegoldii | | stercoris | | distasonis | |
| N-palmitoyl- | (15267) G: | −0.07432 | (4577) S: | 0.043384 | (15315) G: | −0.03035 |
| heptadecasphingosine | Firmicutes | | Coprococcus | | Faecalibacterium | |
| (d17:1/16:0)* | unclassified | | comes | | | |
| beta-alanine | (6179) G: | 0.036483 | (4303) S: | 0.030644 | (4868) S: | 0.021474 |
| | Clostridium | | Clostridium sp | | Blautia sp | |
| | | | CAG 217 | | | |
| X - 21474 | (4577) S: | 0.071572 | (1812) S: | 0.070945 | (4964) F: | 0.06871 |
| | Coprococcus | | Bacteroides | | Eubacteriaceae | |
| | comes | | massiliensis | | | |
| 2- | (3957) F: | −0.08473 | (4782) U: | −0.07791 | (15332) S: | 0.065545 |
| docosahexaenoylglycerol | Lachnospiraceae | | Unknown | | Faecalibacterium | |
| (22:6)* | | | | | prausnitzii | |
| margarate | (6174) S: | 0.031011 | (5111) S: | 0.018587 | (14823) F: | 0.016009 |
| (17:0) | Clostridium sp | | Clostridium sp | | Eggerthellaceae | |
| | CAG 265 | | CAG 127 | | | |
| 1-ribosyl- | (4532) S: | 0.033469 | (10068) S: | −0.02988 | (15299) G: | 0.027021 |
| imidazoleacetate* | Eubacterium | | Escherichia | | Gemmiger | |
| | hallii | | coli | | | |
| X - 21295 | (15124) F: | −0.03467 | (14963) S: | −0.03408 | (15317) S: | −0.02242 |
| | Clostridiales | | Anaerotruncus | | Faecalibacterium | |
| | unclassified | | colihominis | | sp | |
| | | | | | CAG 82 | |
| cysteinylglycine | (4705) S: | 0.050438 | (4820) S: | −0.03788 | (17256) S: | −0.02435 |
| disulfide* | Clostridium sp | | Blautia sp | | Bifidobacterium | |
| | CAG 43 | | | | bifidum | |
| tryptophan | (7044) S: | 0.024669 | (6148) F: | 0.024094 | (6179) G: | 0.014877 |
| | Lactobacillus | | Peptostrep- | | Clostridium | |
| | acidophilus | | tococcaceae | | | |
| 1-palmitoyl-2- | (4448) G: | 0.024707 | (4804) S: | −0.02467 | (15385) U: | −0.02404 |
| docosahexaenoyl- | Eubacterium | | Blautia sp | | Unknown | |
| GPE | | | | | | |
| (16:0/22:6)* | | | | | | |
| S- | (4780) G: | −0.06198 | (9226) S: | −0.06129 | (5087) S: | 0.049888 |
| adenosylhomocysteine | Clostridium | | Akkermansia | | Eubacterium | |
| (SAH) | | | muciniphila | | sp CAG | |
| | | | | | 86 | |
| X - 12206 | (6754) S: | 0.045905 | (4804) S: | −0.04158 | (14542) G: | −0.02963 |
| | Clostridium sp | | Blautia sp | | Collinsella | |
| X - 18345 | (6750) S: | 0.04425 | (4581) S: | 0.0225 | (6340) S: | 0.021425 |
| | Clostridium sp | | Dorea | | Clostridium | |
| | | | longicatena | | sp CAG | |
| | | | | | 269 | |
| tauro-beta- | (4198) S: | 0.059583 | (6376) F: | 0.044695 | (4988) S: | 0.037286 |
| muricholate | Eubacterium | | Clostridiaceae | | Eisenbergiella | |
| | siraeum | | | | tayi | |
| phenylpyruvate | (14797) G: | −0.0131 | (4829) S: | −0.00948 | (14250) U: | −0.00898 |
| | Adlercreutzia | | Blautia sp | | Unknown | |
| oleoyl | (1861) S: | 0.025815 | (17244) S: | −0.02307 | (6174) S: | 0.020522 |
| ethanolamide | Bacteroides | | Bifidobacterium | | Clostridium | |
| | thetaiotaomicron | | adolescentis | | sp CAG | |
| | | | | | 265 | |
| 2,3- | (5121) S: | 0.032593 | (6179) G: | 0.028257 | (4940) S: | −0.0211 |
| dihydroxyisovalerate | Clostridium sp | | Clostridium | | Roseburia | |
| | CAG 264 | | | | inulinivorans | |
| X - 16964 | (6367) F: | 0.144376 | (2318) S: | −0.08445 | (15154) F: | 0.067187 |
| | Clostridiaceae | | Alistipes | | Clostridiales | |
| | | | putredinis | | unclassified | |
| X - 12544 | (14816) F: | 0.070887 | (14815) F: | 0.028234 | (1815) S: | −0.01229 |
| | Eggerthellaceae | | Eggerthellaceae | | Bacteroides | |
| | | | | | dorei | |
| arachidate | (14974) U: | 0.034997 | (15073) G: | 0.034994 | (4826) S: | −0.02028 |
| (20:0) | Unknown | | Oscillibacter | | Blautia sp | |
| X - 17655 | (14991) F: | 0.028822 | (1934) S: | −0.01445 | (14853) S: | −0.01095 |
| | Clostridiaceae | | Parabacteroides | | Clostridium | |
| | | | distasonis | | leptum | |
| 5alpha- | (15089) S: | −0.0453 | (15216) F: | 0.037816 | (15106) S: | 0.026238 |
| pregnan- | Firmicutes | | Clostridiales | | Firmicutes | |
| 3beta,20alpha- | bacterium | | unclassified | | bacterium | |
| diol disulfate | CAG 83 | | | | CAG 176 | |
| X - 15486 | (4816) S: | −0.0387 | (4540) S: | −0.03523 | (4564) S: | 0.022071 |
| | Blautia sp | | Anaerostipes | | Ruminococcus | |
| | | | hadrus | | torques | |
| 3,7- | (14921) U: | 0.072531 | (4644) S: | −0.03999 | (7044) S: | 0.018543 |
| dimethylurate | Unknown | | Clostridium sp | | Lactobacillus | |
| | | | CAG 62 | | acidophilus | |

| | Top | Directional | Top | Directional | | |
|---|---|---|---|---|---|---|
| | predictor | SHAP value | predictor | SHAP value | Microbiome | Microbiome |
| BIOCHEMICAL | #4 | #4 | #5 | #5 | Pearson R | p-value |
| X - 16124 | (14807) S: | −0.02307 | (1832) S: | −0.02073 | 0.797711 | 5.83E−106 |
| | Gordonibacter | | Bacteroides | | | |
| | pamelaeae | | clarus | | | |
| X - 11850 | (15091) G: | 0.074793 | (15356) U: | 0.050296 | 0.710316 | 3.80E−74 |
| | Oscillibacter | | Unknown | | | |
| X - 11843 | (15356) U: | 0.082472 | (15091) G: | 0.054271 | 0.666618 | 2.39E−62 |
| | Unknown | | Oscillibacter | | | |
| X - 12261 | (14924) S: | 0.040316 | (15403) U: | 0.022024 | 0.652153 | 7.18E−59 |
| | Firmicutes | | Unknown | | | |
| | bacterium | | | | | |
| | CAG 137 | | | | | |
| X - 12013 | (15356) U: | 0.076366 | (15090) S: | 0.054881 | 0.648938 | 4.01E−58 |
| | Unknown | | Oscillibacter | | | |
| | | | sp CAG | | | |
| | | | 241 | | | |
| p-cresol- | (15216) F: | 0.064128 | (15236) G: | 0.05707 | 0.634979 | 5.55E−55 |
| glucuronide* | Clostridiales | | Firmicutes | | | |
| | unclassified | | unclassified | | | |
| phenylacetylglutamine | (15271) S: | 0.044504 | (15236) G: | 0.043223 | 0.605077 | 9.03E−49 |
| | Ruthenibacterium | | Firmicutes | | | |
| | lactatiformans | | unclassified | | | |
| p-cresol sulfate | (15078) S: | 0.050134 | (15234) S: | 0.048424 | 0.588586 | 1.28E−45 |
| | Oscillibacter | | Firmicutes | | | |
| | sp | | bacterium | | | |
| | | | CAG 124 | | | |
| phenylacetate | (15216) F: | 0.051491 | (15271) S: | 0.03868 | 0.564933 | 2.12E−41 |
| | Clostridiales | | Ruthenibacterium | | | |
| | unclassified | | lactatiformans | | | |
| X - 12816 | (4648) G: | 0.064364 | (4933) S: | −0.06138 | 0.557555 | 3.75E−40 |
| | Roseburia | | Eubacterium | | | |
| | | | rectale | | | |
| quinate | (15295) G: | 0.050549 | (14921) U: | 0.039571 | 0.550659 | 5.16E−39 |
| | Gemmiger | | Unknown | | | |
| 1-methylurate | (14322) S: | 0.079084 | (1861) S: | −0.06844 | 0.543234 | 8.11E−38 |
| | Eggerthella sp | | Bacteroides | | | |
| | CAG 209 | | thetaiotaomicron | | | |
| X - 24811 | (4537) S: | −0.08525 | (4961) G: | 0.072597 | 0.538398 | 4.70E−37 |
| | Eubacterium | | Eubacterium | | | |
| | hallii | | | | | |
| 5-acetylamino- | (14322) S: | 0.073067 | (4714) S: | −0.05909 | 0.525784 | 4.03E−35 |
| 6-amino-3- | Eggerthella sp | | Clostridium | | | |
| methyluracil | CAG 209 | | sp | | | |
| 1- | (14322) S: | 0.067591 | (14993) S: | 0.066044 | 0.522308 | 1.33E−34 |
| methylxanthine | Eggerthella sp | | Butyricicoccus | | | |
| | CAG 209 | | sp | | | |
| 1,7- | (4781) U: | 0.059593 | (4714) S: | −0.05708 | 0.516272 | 1.03E−33 |
| dimethylurate | Unknown | | Clostridium | | | |
| | | | sp | | | |
| cinnamoylglycine | (15332) S: | −0.06417 | (15234) S: | 0.054896 | 0.507231 | 2.02E−32 |
| | Faecalibacterium | | Firmicutes | | | |
| | prausnitzii | | bacterium | | | |
| | | | CAG 124 | | | |
| X - 12126 | (15031) S: | 0.051916 | (14306) S: | 0.050913 | 0.506864 | 2.28E−32 |
| | Firmicutes | | Clostridium | | | |
| | bacterium | | sp CAG | | | |
| | CAG 110 | | 138 | | | |
| 1,3- | (15300) S: | −0.06312 | (4960) G: | −0.06277 | 0.506154 | 2.87E−32 |
| dimethylurate | Gemmiger | | Eubacterium | | | |
| | formicilis | | | | | |
| theophylline | (1861) S: | −0.06265 | (4960) G: | −0.06141 | 0.500431 | 1.80E−31 |
| | Bacteroides | | Eubacterium | | | |
| | thetaiotaomicron | | | | | |
| paraxanthine | (4581) S: | 0.118133 | (4537) S: | −0.08627 | 0.494815 | 1.05E−30 |
| | Dorea | | Eubacterium | | | |
| | longicatena | | hallii | | | |
| X - 21442 | (15085) F: | 0.072256 | (4828) S: | 0.072119 | 0.48591 | 1.63E−29 |
| | Clostridiales | | Blautia sp | | | |
| | unclassified | | | | | |
| 1,3,7- | (15295) G: | 0.081972 | (1861) S: | −0.07762 | 0.481535 | 6.07E−29 |
| trimethylurate | Gemmiger | | Bacteroides | | | |
| | | | thetaiotaomicron | | | |
| X - 12851 | (6783) S: | −0.05044 | (4659) S: | −0.04491 | 0.479291 | 1.18E−28 |
| | Catenibacterium | | Clostridium | | | |
| | sp CAG | | sp CAG | | | |
| | 290 | | 122 | | | |
| caffeine | (4781) U: | 0.062834 | (14921) U: | 0.043182 | 0.479016 | 1.28E−28 |
| | Unknown | | Unknown | | | |
| X - 12216 | (15356) U: | 0.051318 | (15271) S: | 0.042737 | 0.47398 | 5.63E−28 |
| | Unknown | | Ruthenibacterium | | | |
| | | | lactatiformans | | | |
| N-acetyl- | (5090) S: | −0.0408 | (2301) S: | 0.035489 | 0.464233 | 9.20E−27 |
| cadaverine | Clostridiales | | Alistipes | | | |
| | bacterium | | finegoldii | | | |
| | KLE1615 | | | | | |
| 3- | (4782) U: | 0.042376 | (15234) S: | 0.036809 | 0.463566 | 1.11E−26 |
| phenylpropionate | Unknown | | Firmicutes | | | |
| (hydrocinnamate) | | | bacterium | | | |
| | | | CAG 124 | | | |
| glycolithocholate | (2318) S: | 0.050941 | (14807) S: | 0.047899 | 0.45829 | 4.84E−26 |
| sulfate* | Alistipes | | Gordonibacter | | | |
| | putredinis | | pamelaeae | | | |
| phenylacetylcarnitine | (15244) F: | 0.060642 | (15385) U: | 0.055794 | 0.452403 | 2.43E−25 |
| | Clostridiales | | Unknown | | | |
| | unclassified | | | | | |
| isoursodeoxycholate | (4749) S: | 0.057406 | (15054) F: | −0.0571 | 0.4503 | 4.28E−25 |
| | Clostridium sp | | Clostridiales | | | |
| | CAG 7 | | unclassified | | | |
| X - 12837 | (15395) U: | 0.080074 | (5065) S: | 0.055587 | 0.449837 | 4.85E−25 |
| | Unknown | | Butyrivibrio | | | |
| | | | crossotus | | | |
| X - 24410 | (4882) S: | −0.05591 | (14575) G: | 0.04456 | 0.444238 | 2.16E−24 |
| | Roseburia sp | | Collinsella | | | |
| | CAG 100 | | | | | |
| 5alpha- | (15196) F: | 0.05097 | (1957) S: | 0.049039 | 0.437404 | 1.28E−23 |
| androstan- | Clostridiales | | Bacteroides | | | |
| 3beta,17alpha- | unclassified | | sp CAG | | | |
| diol disulfate | | | 144 | | | |
| X - 21821 | (4608) S: | −0.05078 | (4540) S: | 0.050724 | 0.433422 | 3.56E−23 |
| | Ruminococcus | | Anaerostipes | | | |
| | torques | | hadrus | | | |
| 3-methyl | (14993) S: | 0.044922 | (15315) G: | −0.04294 | 0.430459 | 7.55E−23 |
| catechol | Butyricicoccus | | Faecalibacterium | | | |
| sulfate (1) | sp | | | | | |
| X - 17612 | (15216) F: | 0.057974 | (3940) U: | 0.057185 | 0.42642 | 2.08E−22 |
| | Clostridiales | | Unknown | | | |
| | unclassified | | | | | |
| 3- | (15295) G: | 0.052344 | (4961) G: | 0.047287 | 0.421956 | 6.25E−22 |
| hydroxypyridine | Gemmiger | | Eubacterium | | | |
| sulfate | | | | | | |
| X - 23655 | (4537) S: | −0.08553 | (15295) G: | 0.081629 | 0.419593 | 1.11E−21 |
| | Eubacterium | | Gemmiger | | | |
| | hallii | | | | | |
| X - 17351 | (4608) S: | −0.04254 | (4711) F: | 0.041302 | 0.4165 | 2.35E−21 |
| | Ruminococcus | | Clostridiaceae | | | |
| | torques | | | | | |
| X - 23997 | (3957) F: | 0.079964 | (15078) S: | 0.063895 | 0.41358 | 4.74E−21 |
| | Lachnospiraceae | | Oscillibacter | | | |
| | | | sp | | | |
| 4- | (15315) G: | −0.05002 | (15295) G: | 0.047266 | 0.413294 | 5.07E−21 |
| ethylcatechol | Faecalibacterium | | Gemmiger | | | |
| sulfate | | | | | | |
| X - 13729 | (14921) U: | 0.037359 | (5117) S: | −0.03645 | 0.412317 | 6.40E−21 |
| | Unknown | | Coprococcus | | | |
| | | | eutactus | | | |
| ursodeoxycholate | (6367) F: | −0.07313 | (2325) S: | −0.06835 | 0.412223 | 6.54E−21 |
| | Clostridiaceae | | Alistipes | | | |
| | | | indistinctus | | | |
| taurolithocholate | (6148) F: | −0.03866 | (4552) S: | 0.0376 | 0.409121 | 1.36E−20 |
| 3-sulfate | Peptostrep- | | Ruminococcus | | | |
| | tococcaceae | | sp | | | |
| X - 17469 | (4705) S: | 0.05358 | (4938) S: | 0.050476 | 0.405438 | 3.21E−20 |
| | Clostridium sp | | Roseburia | | | |
| | CAG 43 | | sp | | | |
| X - 23649 | (15073) G: | 0.122182 | (4537) S: | −0.10713 | 0.405318 | 3.30E−20 |
| | Oscillibacter | | Eubacterium | | | |
| | | | hallii | | | |
| 4- | (9226) S: | 0.027671 | (15271) S: | 0.026973 | 0.403773 | 4.72E−20 |
| methylcatechol | Akkermansia | | Ruthenibacterium | | | |
| sulfate | muciniphila | | lactatiformans | | | |
| indolepropionate | (4714) S: | 0.046985 | (4584) S: | −0.04353 | 0.402571 | 6.23E−20 |
| | Clostridium sp | | Ruminococcus | | | |
| | | | gnavus | | | |
| citraconate/ | (4537) S: | −0.05218 | (14322) S: | 0.051828 | 0.397921 | 1.80E−19 |
| glutaconate | Eubacterium | | Eggerthella | | | |
| | hallii | | sp CAG | | | |
| | | | 209 | | | |
| X - 21752 | (4782) U: | 0.051235 | (15467) S: | −0.03926 | 0.397715 | 1.88E−19 |
| | Unknown | | Desulfovibrio | | | |
| | | | piger | | | |
| X - 24243 | (14624) G: | 0.053135 | (1867) S: | −0.05313 | 0.397632 | 1.92E−19 |
| | Collinsella | | Bacteroides | | | |
| | | | xylanisolvens | | | |
| 1-(1-enyl- | (4782) U: | −0.05491 | (15370) F: | −0.05439 | 0.390492 | 9.43E−19 |
| palmitoyl)-2- | Unknown | | Ruminococcaceae | | | |
| arachidonoyl- | | | | | | |
| GPE | | | | | | |
| (P-16:0/20:4)* | | | | | | |
| 5alpha- | (15350) U: | 0.068515 | (4940) S: | 0.058214 | 0.388836 | 1.36E−18 |
| androstan- | Unknown | | Roseburia | | | |
| 3alpha,17beta- | | | inulinivorans | | | |
| diol | | | | | | |
| monosulfate | | | | | | |
| (2) | | | | | | |
| hippurate | (15085) F: | 0.037015 | (4537) S: | −0.03406 | 0.388495 | 1.46E−18 |
| | Clostridiales | | Eubacterium | | | |
| | unclassified | | hallii | | | |
| 5- | (15403) U: | 0.073587 | (15236) G: | 0.058161 | 0.383075 | 4.74E−18 |
| hydroxyhexanoate | Unknown | | Firmicutes | | | |
| | | | unclassified | | | |
| indolin-2-one | (9283) S: | −0.03591 | (14999) U: | 0.033183 | 0.382394 | 5.49E−18 |
| | Sutterella | | Unknown | | | |
| | wadsworthensis | | | | | |
| X - 17145 | (4960) G: | 0.051903 | (4933) S: | −0.0451 | 0.381408 | 6.77E−18 |
| | Eubacterium | | Eubacterium | | | |
| | | | rectale | | | |
| 2,3- | (14921) U: | 0.108246 | (4960) G: | −0.10493 | 0.38057 | 8.10E−18 |
| dihydroxypyridine | Unknown | | Eubacterium | | | |
| X - 17354 | (4575) S: | −0.05936 | (15236) G: | 0.057047 | 0.376788 | 1.80E−17 |
| | Dorea | | Firmicutes | | | |
| | formicigenerans | | unclassified | | | |
| glycodeoxycholate | (4659) S: | −0.08326 | (15143) S: | 0.083256 | 0.376238 | 2.03E−17 |
| | Clostridium sp | | Flavonifractor | | | |
| | CAG 122 | | sp | | | |
| X - 23639 | (14322) S: | 0.047223 | (6962) S: | −0.04332 | 0.373791 | 3.38E−17 |
| | Eggerthella sp | | Megamonas | | | |
| | CAG 209 | | funiformis | | | |
| 6- | (9283) S: | −0.03609 | (14999) U: | 0.035525 | 0.370164 | 7.15E−17 |
| hydroxyindole | Sutterella | | Unknown | | | |
| sulfate | wadsworthensis | | | | | |
| X - 12306 | (6747) S: | −0.04692 | (4581) S: | −0.04658 | 0.365101 | 2.01E−16 |
| | Clostridium | | Dorea | | | |
| | spiroforme | | longicatena | | | |
| phenol sulfate | (15254) F: | −0.03453 | (17244) S: | −0.02993 | 0.363893 | 2.56E−16 |
| | Clostridiales | | Bifidobacterium | | | |
| | unclassified | | adolescentis | | | |
| 5-acetylamino- | (4914) S: | −0.0518 | (4537) S: | −0.04073 | 0.363687 | 2.67E−16 |
| 6-formylamino- | Clostridium sp | | Eubacterium | | | |
| 3-methyluracil | | | hallii | | | |
| 1,5- | (5068) S: | −0.03729 | (4924) G: | −0.03657 | 0.362851 | 3.15E−16 |
| anhydroglucitol | Bacteroides | | Roseburia | | | |
| (1,5-AG) | pectinophilus | | | | | |
| | CAG 437 | | | | | |
| N- | (4779) S: | 0.044408 | (4540) S: | −0.04234 | 0.361999 | 3.74E−16 |
| acetylcarnosine | Clostridium sp | | Anaerostipes | | | |
| | | | hadrus | | | |
| 3-indoxyl | (14999) U: | 0.031692 | (14853) S: | 0.029993 | 0.358283 | 7.82E−16 |
| sulfate | Unknown | | Clostridium | | | |
| | | | leptum | | | |
| maleate | (14807) S: | −0.06106 | (15295) G: | 0.056944 | 0.355938 | 1.24E−15 |
| | Gordonibacter | | Gemmiger | | | |
| | pamelaeae | | | | | |
| L-urobilin | (14311) F: | 0.047228 | (14909) S: | 0.043864 | 0.354595 | 1.61E−15 |
| | Clostridiaceae | | Clostridium | | | |
| | | | sp CAG | | | |
| | | | 169 | | | |
| X - 21286 | (15234) S: | 0.044237 | (6140) S: | −0.0435 | 0.351181 | 3.11E−15 |
| | Firmicutes | | Intestinibacter | | | |
| | bacterium | | bartlettii | | | |
| | CAG 124 | | | | | |
| X - 12718 | (15271) S: | 0.050914 | (5190) S: | 0.049193 | 0.350751 | 3.38E−15 |
| | Ruthenibacterium | | Firmicutes | | | |
| | lactatiformans | | bacterium | | | |
| | | | CAG 102 | | | |
| carotene diol | (4705) S: | −0.06239 | (4581) S: | −0.04134 | 0.350531 | 3.53E−15 |
| (2) | Clostridium sp | | Dorea | | | |
| | CAG 43 | | longicatena | | | |
| X - 21310 | (4581) S: | 0.04046 | (6754) S: | −0.03592 | 0.349667 | 4.16E−15 |
| | Dorea | | Clostridium | | | |
| | longicatena | | sp | | | |
| X - 14662 | (4862) S: | 0.036385 | (4925) S: | 0.036061 | 0.346125 | 8.16E−15 |
| | Blautia sp | | Roseburia | | | |
| | CAG 257 | | faecis | | | |
| glycoursodeoxycholate | (4552) S: | −0.04884 | (9226) S: | −0.0399 | 0.343447 | 1.35E−14 |
| | Ruminococcus | | Akkermansia | | | |
| | sp | | muciniphila | | | |
| X - 12283 | (4940) S: | −0.04039 | (14861) U: | 0.03973 | 0.342573 | 1.59E−14 |
| | Roseburia | | Unknown | | | |
| | inulinivorans | | | | | |
| X - 11315 | (4651) S: | 0.036928 | (4964) F: | 0.03536 | 0.339457 | 2.83E−14 |
| | Clostridium sp | | Eubacteriaceae | | | |
| | CAG 230 | | | | | |
| trigonelline | (1861) S: | −0.0463 | (14921) U: | 0.034628 | 0.338307 | 3.50E−14 |
| (N′- | Bacteroides | | Unknown | | | |
| methylnicotinate) | thetaiotaomicron | | | | | |
| X - 16654 | (9347) S: | 0.037811 | (15322) S: | −0.03683 | 0.338005 | 3.70E−14 |
| | Azospirillum | | Faecalibacterium | | | |
| | sp CAG 260 | | prausnitzii | | | |
| X - 22162 | (15081) F: | 0.046343 | (15369) S: | 0.045969 | 0.336432 | 4.93E−14 |
| | Clostridiales | | Faecalibacterium | | | |
| | unclassified | | sp | | | |
| | | | CAG 74 | | | |
| X - 12329 | (4537) S: | −0.07682 | (4961) G: | 0.066374 | 0.336052 | 5.28E−14 |
| | Eubacterium | | Eubacterium | | | |
| | hallii | | | | | |
| ergothioneine | (4714) S: | 0.04008 | (4581) S: | −0.03351 | 0.333717 | 8.07E−14 |
| | Clostridium sp | | Dorea | | | |
| | | | longicatena | | | |
| anthranilate | (4706) F: | −0.05931 | (1814) S: | −0.04385 | 0.331065 | 1.30E−13 |
| | Clostridiaceae | | Bacteroides | | | |
| | | | vulgatus | | | |
| cholate | (5190) S: | −0.03369 | (6140) S: | 0.026256 | 0.327602 | 2.40E−13 |
| | Firmicutes | | Intestinibacter | | | |
| | bacterium | | bartlettii | | | |
| | CAG 102 | | | | | |
| 4- | (15229) F: | 0.041976 | (4909) G: | 0.041059 | 0.327393 | 2.49E−13 |
| hydroxycoumarin | Clostridiales | | Clostridium | | | |
| | unclassified | | | | | |
| X - 11880 | (4581) S: | 0.046825 | (15271) S: | −0.03851 | 0.326318 | 3.01E−13 |
| | Dorea | | Ruthenibacterium | | | |
| | longicatena | | lactatiformans | | | |
| X - 22509 | (15369) S: | 0.038586 | (15054) F: | 0.034081 | 0.320452 | 8.35E−13 |
| | Faecalibacterium | | Clostridiales | | | |
| | sp CAG 74 | | unclassified | | | |
| 1-lignoceroyl- | (15332) S: | −0.0409 | (14991) F: | 0.040444 | 0.320154 | 8.79E−13 |
| GPC (24:0) | Faecalibacterium | | Clostridiaceae | | | |
| | prausnitzii | | | | | |
| N2,N5- | (14501) S: | −0.04328 | (4714) S: | 0.041264 | 0.318295 | 1.21E−12 |
| diacetylornithine | Collinsella | | Clostridium | | | |
| | aerofaciens | | sp | | | |
| 3-methyl | (4537) S: | −0.07471 | (15154) F: | 0.069279 | 0.314349 | 2.36E−12 |
| catechol | Eubacterium | | Clostridiales | | | |
| sulfate (2) | hallii | | unclassified | | | |
| glutarate | (4564) S: | 0.031254 | (4782) U: | −0.03113 | 0.313428 | 2.75E−12 |
| (pentanedioate) | Ruminococcus | | Unknown | | | |
| | torques | | | | | |
| X - 18249 | (4834) G: | −0.04302 | (4826) S: | 0.042088 | 0.311953 | 3.52E−12 |
| | Blautia | | Blautia sp | | | |
| methyl | (1963) S: | −0.0403 | (15132) S: | −0.03755 | 0.309776 | 5.05E−12 |
| glucopyranoside | Coprobacter | | Flavonifractor | | | |
| (alpha + | fastidiosus | | plautii | | | |
| beta) | | | | | | |
| 7- | (15342) S: | 0.048362 | (2295) S: | −0.04184 | 0.307995 | 6.77E−12 |
| methylguanine | Faecalibacterium | | Alistipes | | | |
| | prausnitzii | | shahii | | | |
| X - 11308 | (17244) S: | 0.05329 | (9226) S: | −0.04105 | 0.307272 | 7.62E−12 |
| | Bifidobacterium | | Akkermansia | | | |
| | adolescentis | | muciniphila | | | |
| X - 12738 | (15154) F: | 0.068911 | (14861) U: | 0.064852 | 0.302725 | 1.59E−11 |
| | Clostridiales | | Unknown | | | |
| | unclassified | | | | | |
| gentisate | (4714) S: | 0.037631 | (15132) S: | −0.03215 | 0.300645 | 2.22E−11 |
| | Clostridium sp | | Flavonifractor | | | |
| | | | plautii | | | |
| carotene diol | (4816) S: | 0.047076 | (4564) S: | −0.04206 | 0.295736 | 4.83E−11 |
| (1) | Blautia sp | | Ruminococcus | | | |
| | | | torques | | | |
| 5alpha- | (15332) S: | 0.068124 | (5736) S: | 0.052653 | 0.291715 | 9.02E−11 |
| androstan- | Faecalibacterium | | Acidaminococcus | | | |
| 3alpha,17beta- | prausnitzii | | intestini | | | |
| diol disulfate | | | | | | |
| X - 11372 | (3964) U: | −0.03458 | (4581) S: | 0.033513 | 0.290936 | 1.02E−10 |
| | Unknown | | Dorea | | | |
| | | | longicatena | | | |
| X - 17185 | (4961) G: | 0.057189 | (14861) U: | 0.046666 | 0.29084 | 1.03E−10 |
| | Eubacterium | | Unknown | | | |
| X - 23652 | (4925) S: | 0.028414 | (2303) S: | −0.02823 | 0.290612 | 1.07E−10 |
| | Roseburia | | Alistipes | | | |
| | faecis | | finegoldii | | | |
| X - 18240 | (1798) S: | 0.041467 | (6140) S: | −0.04124 | 0.289972 | 1.18E−10 |
| | Paraprevotella | | Intestinibacter | | | |
| | clara | | bartlettii | | | |
| X - 18914 | (6139) G: | −0.04465 | (5075) S: | 0.043974 | 0.289125 | 1.34E−10 |
| | Intestinibacter | | Lachnospira | | | |
| | | | pectinoschiza | | | |
| X - 22520 | (6962) S: | 0.057831 | (2295) S: | −0.05649 | 0.287183 | 1.80E−10 |
| | Megamonas | | Alistipes | | | |
| | funiformis | | shahii | | | |
| 3-(3- | (6359) F: | 0.047664 | (4771) G: | −0.0456 | 0.286363 | 2.04E−10 |
| hydroxyphe- | Clostridiaceae | | Clostridium | | | |
| nyl)propionate | | | | | | |
| dimethyl | (4669) G: | 0.043094 | (8010) S: | −0.03642 | 0.286074 | 2.13E−10 |
| sulfoxide | Coprococcus | | Streptococcus | | | |
| (DMSO) | | | salivarius | | | |
| threonate | (6367) F: | 0.042789 | (4564) S: | −0.03099 | 0.283418 | 3.17E−10 |
| | Clostridiaceae | | Ruminococcus | | | |
| | | | torques | | | |
| X - 12730 | (15073) G: | 0.061366 | (15154) F: | 0.058999 | 0.283413 | 3.17E−10 |
| | Oscillibacter | | Clostridiales | | | |
| | | | unclassified | | | |
| X - 19434 | (1845) S: | −0.02893 | (6174) S: | 0.025256 | 0.281736 | 4.07E−10 |
| | Bacteroides | | Clostridium | | | |
| | intestinalis | | sp CAG | | | |
| | CAG 315 | | 265 | | | |
| X - 24948 | (15317) S: | 0.038143 | (3964) U: | −0.03749 | 0.281466 | 4.24E−10 |
| | Faecalibacterium | | Unknown | | | |
| | sp CAG 82 | | | | | |
| 1-(1-enyl- | (4557) S: | 0.057641 | (4712) F: | −0.05646 | 0.280904 | 4.61E−10 |
| stearoyl)-2- | Ruminococcus | | Clostridiaceae | | | |
| arachidonoyl- | lactaris | | | | | |
| GPE | | | | | | |
| (P-18:0/20:4)* | | | | | | |
| X - 23659 | (14992) G: | 0.045353 | (6367) F: | 0.036691 | 0.280788 | 4.69E−10 |
| | Butyricicoccus | | Clostridiaceae | | | |
| 5alpha- | (15315) G: | 0.046046 | (4581) S: | 0.043609 | 0.280509 | 4.88E−10 |
| androstan- | Faecalibacterium | | Dorea | | | |
| 3alpha,17alpha- | | | longicatena | | | |
| diol | | | | | | |
| monosulfate | | | | | | |
| X - 21339 | (5736) S: | 0.031775 | (4581) S: | 0.031682 | 0.280474 | 4.91E−10 |
| | Acidaminococcus | | Dorea | | | |
| | intestini | | longicatena | | | |
| 4- | (3940) U: | 0.024509 | (4964) F: | 0.021158 | 0.277385 | 7.72E−10 |
| ethylphenylsulfate | Unknown | | Eubacteriaceae | | | |
| gamma- | (6754) S: | −0.06004 | (8002) S: | 0.057386 | 0.27641 | 8.89E−10 |
| glutamylvaline | Clostridium sp | | Streptococcus | | | |
| | | | thermophilus | | | |
| beta- | (4714) S: | 0.033087 | (4750) G: | −0.02983 | 0.276078 | 9.33E−10 |
| cryptoxanthin | Clostridium sp | | Clostridium | | | |
| sphingomyelin | (4893) S: | −0.03112 | (15154) F: | 0.025029 | 0.274995 | 1.09E−09 |
| (d18:1/14:0, | Clostridium sp | | Clostridiales | | | |
| d16:1/16:0)* | | | unclassified | | | |
| X - 21736 | (15132) S: | 0.043581 | (15452) S: | 0.036154 | 0.274785 | 1.12E−09 |
| | Flavonifractor | | Bilophila | | | |
| | plautii | | sp 4 1 30 | | | |
| O-methylcatechol | (4957) F: | 0.052341 | (15315) G: | −0.03872 | 0.273918 | 1.27E−09 |
| sulfate | Eubacteriaceae | | Faecalibacterium | | | |
| N-(2- | (4537) S: | −0.05892 | (15073) G: | 0.047175 | 0.272049 | 1.66E−09 |
| furoyl)glycine | Eubacterium | | Oscillibacter | | | |
| | hallii | | | | | |
| sphingomyelin | (15154) F: | 0.043901 | (15271) S: | 0.034396 | 0.270071 | 2.20E−09 |
| (d17:2/16:0, | Clostridiales | | Ruthenibacterium | | | |
| d18:2/15:0)* | unclassified | | lactatiformans | | | |
| 3- | (4925) S: | 0.024555 | (4643) S: | −0.02344 | 0.268147 | 2.89E−09 |
| methylhistidine | Roseburia | | Clostridium | | | |
| | faecis | | sp CAG | | | |
| | | | 167 | | | |
| X - 13835 | (2303) S: | −0.03515 | (4705) S: | 0.031703 | 0.26789 | 2.99E−09 |
| | Alistipes | | Clostridium | | | |
| | finegoldii | | sp CAG | | | |
| | | | 43 | | | |
| propionylcarnitine | (2303) S: | −0.0446 | (1626) S: | 0.041882 | 0.266513 | 3.63E−09 |
| (C3) | Alistipes | | Prevotella | | | |
| | finegoldii | | copri | | | |
| 3- | (15028) G: | −0.04153 | (14322) S: | 0.038473 | 0.26643 | 3.67E−09 |
| hydroxyhippurate | Firmicutes | | Eggerthella | | | |
| | unclassified | | sp CAG | | | |
| | | | 209 | | | |
| X - 11640 | (15196) F: | 0.026176 | (15369) S: | 0.019358 | 0.264807 | 4.60E−09 |
| | Clostridiales | | Faecalibacterium sp | | | |
| | unclassified | | CAG 74 | | | |
| 3-acetylphenol | (14807) S: | −0.08154 | (15154) F: | 0.069366 | 0.259657 | 9.30E−09 |
| sulfate | Gordonibacter | | Clostridiales | | | |
| | pamelaeae | | unclassified | | | |
| myo-inositol | (6367) F: | 0.042731 | (4810) S: | 0.041682 | 0.257234 | 1.29E−08 |
| | Clostridiaceae | | Blautia sp | | | |
| | | | CAG 237 | | | |
| sphingomyelin | (5089) S: | −0.03696 | (5082) S: | 0.035874 | 0.255615 | 1.60E−08 |
| (d18:2/23:1)* | Eubacterium | | Eubacterium | | | |
| | sp CAG 38 | | eligens | | | |
| 2-naphthol | (1949) S: | 0.054633 | (4581) S: | 0.040863 | 0.255159 | 1.70E−08 |
| sulfate | Parabacteroides | | Dorea | | | |
| | merdae | | longicatena | | | |
| N-delta- | (15291) F: | 0.024447 | (4575) S: | −0.02209 | 0.254419 | 1.88E−08 |
| acetylornithine | Ruminococcaceae | | Dorea | | | |
| | | | formicigenerans | | | |
| benzoylcarnitine* | (6376) F: | 0.035481 | (6334) F: | 0.028441 | 0.254127 | 1.95E−08 |
| | Clostridiaceae | | Clostridiaceae | | | |
| X - 24473 | (4964) F: | 0.044069 | (4826) S: | −0.04362 | 0.253631 | 2.08E−08 |
| | Eubacteriaceae | | Blautia sp | | | |
| X - 11381 | (14861) U: | 0.032022 | (4959) S: | 0.031907 | 0.253541 | 2.11E−08 |
| | Unknown | | Eubacterium | | | |
| | | | ramulus | | | |
| X - 22834 | (15286) F: | −0.05354 | (4749) S: | 0.039812 | 0.252464 | 2.43E−08 |
| | Ruminococcaceae | | Clostridium | | | |
| | | | sp CAG | | | |
| | | | 7 | | | |
| oxalate | (6367) F: | 0.044268 | (10068) S: | −0.03791 | 0.252363 | 2.46E−08 |
| (ethanedioate) | Clostridiaceae | | Escherichia | | | |
| | | | coli | | | |
| alpha- | (15373) F: | −0.03805 | (4925) S: | 0.033112 | 0.250964 | 2.95E−08 |
| hydroxyisovalerate | Ruminococcaceae | | Roseburia | | | |
| | | | faecis | | | |
| X - 24693 | (4987) S: | −0.04606 | (1812) S: | 0.043868 | 0.2507 | 3.06E−08 |
| | Clostridium sp | | Bacteroides | | | |
| | KLE 1755 | | massiliensis | | | |
| X - 24736 | (4714) S: | 0.079137 | (4575) S: | −0.07629 | 0.246434 | 5.30E−08 |
| | Clostridium sp | | Dorea | | | |
| | | | formicigenerans | | | |
| 1H-indole-7- | (15091) G: | 0.056908 | (4564) S: | 0.030947 | 0.24595 | 5.64E−08 |
| acetic acid | Oscillibacter | | Ruminococcus | | | |
| | | | torques | | | |
| urate | (1814) S: | 0.034955 | (1815) S: | −0.03475 | 0.244634 | 6.67E−08 |
| | Bacteroides | | Bacteroides | | | |
| | vulgatus | | dorei | | | |
| taurodeoxycholate | (5121) S: | −0.05448 | (15143) S: | 0.043859 | 0.244395 | 6.87E−08 |
| | Clostridium sp | | Flavonifractor | | | |
| | CAG 264 | | sp | | | |
| sphingomyelin | (4581) S: | −0.03447 | (4552) S: | 0.034054 | 0.243856 | 7.36E−08 |
| (d18:2/14:0, | Dorea | | Ruminococcus | | | |
| d18:1/14:)* | longicatena | | sp | | | |
| glycolithocholate | (6328) S: | 0.027553 | (6140) S: | −0.02364 | 0.242929 | 8.27E−08 |
| | Clostridium sp | | Intestinibacter | | | |
| | CAG 492 | | bartlettii | | | |
| X - 15728 | (4828) S: | 0.038362 | (4782) U: | 0.036873 | 0.240255 | 1.16E−07 |
| | Blautia sp | | Unknown | | | |
| creatinine | (5082) S: | −0.03098 | (15216) F: | −0.02912 | 0.239951 | 1.20E−07 |
| | Eubacterium | | Clostridiales | | | |
| | eligens | | unclassified | | | |
| X - 15461 | (14823) F: | 0.032212 | (14999) U: | 0.028796 | 0.239102 | 1.33E−07 |
| | Eggerthellaceae | | Unknown | | | |
| X - 12822 | (15154) F: | 0.032025 | (4706) F: | 0.031506 | 0.238793 | 1.39E−07 |
| | Clostridiales | | Clostridiaceae | | | |
| | unclassified | | | | | |
| 4-allylphenol | (15342) S: | −0.04237 | (4816) S: | 0.039765 | 0.236489 | 1.84E−07 |
| sulfate | Faecalibacterium | | Blautia sp | | | |
| | prausnitzii | | | | | |
| X - 23782 | (15238) S: | −0.03371 | (4905) F: | 0.029554 | 0.23624 | 1.90E−07 |
| | Firmicutes | | Clostridiaceae | | | |
| | bacterium | | | | | |
| | CAG 170 | | | | | |
| X - 12212 | (1934) S: | −0.03851 | (4826) S: | −0.03332 | 0.234166 | 2.44E−07 |
| | Parabacteroides | | Blautia sp | | | |
| | distasonis | | | | | |
| tryptophan | (15342) S: | 0.034674 | (4953) S: | 0.033559 | 0.233846 | 2.54E−07 |
| betaine | Faecalibacterium | | Roseburia | | | |
| | prausnitzii | | sp CAG | | | |
| | | | 182 | | | |
| I-urobilinogen | (15369) S: | −0.02395 | (4882) S: | −0.02222 | 0.232742 | 2.90E−07 |
| | Faecalibacterium | | Roseburia | | | |
| | sp CAG 74 | | sp CAG | | | |
| | | | 100 | | | |
| sphingomyelin | (15271) S: | 0.025408 | (4704) F: | −0.02446 | 0.232659 | 2.93E−07 |
| (d18:1/19:0, | Ruthenibacterium | | Clostridiaceae | | | |
| d19:1/18:0)* | lactatiformans | | | | | |
| 3-carboxy-4- | (17256) S: | −0.02289 | (4303) S: | 0.02171 | 0.232549 | 2.97E−07 |
| methyl-5- | Bifidobacterium | | Clostridium | | | |
| pentyl-2- | bifidum | | sp CAG | | | |
| furanpropionate | | | 217 | | | |
| (3-CMPFP)** | | | | | | |
| X - 16935 | (10130) S: | −0.04385 | (5736) S: | 0.043701 | 0.232166 | 3.11E−07 |
| | Enterobacter | | Acidaminococcus | | | |
| | cloacae | | intestini | | | |
| sphingomyelin | (15154) F: | 0.031681 | (4670) S: | 0.03152 | 0.231765 | 3.26E−07 |
| (d17:1/16:0, | Clostridiales | | Coprococcus | | | |
| d18:1/15:0, | unclassified | | catus | | | |
| d16:1/17:0)* | | | | | | |
| X - 21829 | (14992) G: | 0.038018 | (15236) G: | −0.03395 | 0.231762 | 3.26E−07 |
| | Butyricicoccus | | Firmicutes | | | |
| | | | unclassified | | | |
| cystine | (4810) S: | 0.02337 | (6783) S: | 0.015823 | 0.231219 | 3.48E−07 |
| | Blautia sp | | Catenibacterium | | | |
| | CAG 237 | | sp | | | |
| | | | CAG 290 | | | |
| X - 24475 | (17244) S: | −0.0296 | (4960) G: | 0.026498 | 0.23117 | 3.50E−07 |
| | Bifidobacterium | | Eubacterium | | | |
| | adolescentis | | | | | |
| 1-stearoyl-2- | (15225) F: | −0.02058 | (15332) S: | 0.019803 | 0.229344 | 4.35E−07 |
| docosahexaenoyl-GPC | Clostridiales | | Faecalibacterium | | | |
| (18:0/22:6) | unclassified | | prausnitzii | | | |
| X - 24951 | (4951) S: | 0.03707 | (4940) S: | 0.028743 | 0.229246 | 4.41E−07 |
| | Roseburia | | Roseburia | | | |
| | intestinalis | | inulinivorans | | | |
| X - 24949 | (9226) S: | −0.05326 | (14853) S: | −0.04316 | 0.228719 | 4.69E−07 |
| | Akkermansia | | Clostridium | | | |
| | muciniphila | | leptum | | | |
| 2- | (4951) S: | 0.023966 | (4834) G: | −0.02366 | 0.227499 | 5.41E−07 |
| hydroxylaurate | Roseburia | | Blautia | | | |
| | intestinalis | | | | | |
| X - 12063 | (14027) U: | −0.03582 | (4938) S: | 0.033338 | 0.226197 | 6.31E−07 |
| | Unknown | | Roseburia | | | |
| | | | sp | | | |
| 2-hydroxy-3- | (4951) S: | 0.024429 | (15390) U: | −0.02315 | 0.225874 | 6.55E−07 |
| methylvalerate | Roseburia | | Unknown | | | |
| | intestinalis | | | | | |
| argininate* | (4826) S: | −0.03779 | (14454) G: | −0.02669 | 0.223051 | 9.09E−07 |
| | Blautia sp | | Collinsella | | | |
| indoleacetate | (14909) S: | 0.021906 | (15254) F: | 0.021898 | 0.222408 | 9.78E−07 |
| | Clostridium sp | | Clostridiales | | | |
| | CAG 169 | | unclassified | | | |
| ceramide | (4670) S: | 0.034903 | (1862) S: | 0.031833 | 0.222142 | 1.01E−06 |
| (d18:1/14:0, | Coprococcus | | Bacteroides | | | |
| d16:1/16:0)* | catus | | finegoldii | | | |
| 5alpha- | (4779) S: | 0.039677 | (4940) S: | 0.03937 | 0.220249 | 1.25E−06 |
| androstan- | Clostridium sp | | Roseburia | | | |
| 3beta,17beta- | | | inulinivorans | | | |
| diol disulfate | | | | | | |
| citrulline | (5083) G: | 0.03285 | (14899) U: | 0.028835 | 0.220015 | 1.29E−06 |
| | Eubacterium | | Unknown | | | |
| 1-methyl-5- | (6754) S: | −0.03983 | (15324) G: | 0.032531 | 0.219473 | 1.37E−06 |
| imidazoleacetate | Clostridium sp | | Faecalibacterium | | | |
| X - 12263 | (9333) S: | −0.04652 | (15225) F: | −0.03552 | 0.218947 | 1.45E−06 |
| | Acetobacter | | Clostridiales | | | |
| | sp CAG 267 | | unclassified | | | |
| taurodeoxycholic | (5785) S: | 0.034277 | (15090) S: | 0.033016 | 0.21761 | 1.69E−06 |
| acid 3- | Phascolarctobacterium | | Oscillibacter | | | |
| sulfate | sp | | sp CAG | | | |
| | CAG 266 | | 241 | | | |
| X - 12543 | (15031) S: | −0.03218 | (6359) F: | 0.031796 | 0.216865 | 1.83E−06 |
| | Firmicutes | | Clostridiaceae | | | |
| | bacterium | | | | | |
| | CAG 110 | | | | | |
| sphingomyelin | (5736) S: | −0.04201 | (15229) F: | −0.02633 | 0.215385 | 2.16E−06 |
| (d18:2/21:0, | Acidaminococcus | | Clostridiales | | | |
| d16:2/23:0)* | intestini | | unclassified | | | |
| N- | (15317) S: | 0.013762 | (5843) S: | −0.00948 | 0.215264 | 2.19E−06 |
| acetylmethionine | Faecalibacterium | | Allisonella | | | |
| | sp CAG 82 | | histaminiformans | | | |
| X - 18901 | (15164) F: | 0.018623 | (14575) G: | −0.01604 | 0.213683 | 2.61E−06 |
| | Clostridiales | | Collinsella | | | |
| | unclassified | | | | | |
| 1- | (1862) S: | 0.034608 | (14909) S: | 0.028404 | 0.213441 | 2.68E−06 |
| palmitoylglycerol | Bacteroides | | Clostridium | | | |
| (16:0) | finegoldii | | sp CAG | | | |
| | | | 169 | | | |
| X - 23587 | (4581) S: | 0.038574 | (17248) S: | −0.03613 | 0.212559 | 2.96E−06 |
| | Dorea | | Bifidobacterium | | | |
| | longicatena | | longum | | | |
| androstenediol | (15154) F: | −0.02695 | (3964) U: | −0.02694 | 0.21083 | 3.57E−06 |
| (3beta,17beta) | Clostridiales | | Unknown | | | |
| disulfate (2) | unclassified | | | | | |
| tartronate | (4931) G: | 0.03789 | (14909) S: | −0.032 | 0.210444 | 3.72E−06 |
| (hydroxymalonate) | Lachnospiraceae | | Clostridium | | | |
| | unclassified | | sp CAG | | | |
| | | | 169 | | | |
| X - 24352 | (5087) S: | 0.026932 | (1872) S: | −0.0223 | 0.210313 | 3.78E−06 |
| | Eubacterium | | Bacteroides | | | |
| | sp CAG 86 | | ovatus | | | |
| X - 23654 | (1790) S: | −0.03973 | (4705) S: | 0.038681 | 0.20987 | 3.96E−06 |
| | Odoribacter | | Clostridium | | | |
| | splanchnicus | | sp CAG | | | |
| | | | 43 | | | |
| dihydrocaffeate | (14322) S: | 0.039202 | (1872) S: | −0.03677 | 0.209085 | 4.31E−06 |
| sulfate (2) | Eggerthella sp | | Bacteroides | | | |
| | CAG 209 | | ovatus | | | |
| sphingomyelin | (4670) S: | 0.024397 | (15271) S: | 0.020911 | 0.207739 | 4.98E−06 |
| (d18:1/17:0, | Coprococcus | | Ruthenibacterium | | | |
| d17:1/18:0, | catus | | lactatiformans | | | |
| d19:1/16:0) | | | | | | |
| 3-carboxy-4- | (1798) S: | −0.03169 | (6141) F: | 0.029622 | 0.207713 | 5.00E−06 |
| methyl-5- | Paraprevotella | | Peptostrep- | | | |
| propyl-2- | clara | | tococcaceae | | | |
| furanpropanoate | | | | | | |
| (CMPF) | | | | | | |
| X - 18606 | (15078) S: | −0.02597 | (14993) S: | 0.017171 | 0.207705 | 5.00E−06 |
| | Oscillibacter | | Butyricicoccus sp | | | |
| | sp | | | | | |
| 2,3-dihydroxy- | (1798) S: | 0.033106 | (15131) F: | −0.03174 | 0.207139 | 5.31E−06 |
| 2-methylbutyrate | Paraprevotella | | Clostridiales | | | |
| | clara | | unclassified | | | |
| X - 12221 | (5083) G: | 0.027656 | (4648) G: | 0.026516 | 0.206925 | 5.44E−06 |
| | Eubacterium | | Roseburia | | | |
| X - 14082 | (4957) F: | 0.040458 | (14921) U: | 0.034511 | 0.206394 | 5.75E−06 |
| | Eubacteriaceae | | Unknown | | | |
| X - 13703 | (15295) G: | 0.032892 | (1798) S: | 0.03269 | 0.206145 | 5.91E−06 |
| | Gemmiger | | Paraprevotella | | | |
| | | | clara | | | |
| X - 17676 | (4831) F: | 0.042912 | (14322) S: | 0.030579 | 0.204984 | 6.68E−06 |
| | Lachnospiraceae | | Eggerthella | | | |
| | | | sp CAG | | | |
| | | | 209 | | | |
| X - 24801 | (1862) S: | 0.027108 | (4957) F: | 0.026568 | 0.204124 | 7.31E−06 |
| | Bacteroides | | Eubacteriaceae | | | |
| | finegoldii | | | | | |
| N- | (5089) S: | 0.022055 | (6367) F: | 0.020723 | 0.203976 | 7.43E−06 |
| methylproline | Eubacterium | | Clostridiaceae | | | |
| | sp CAG 38 | | | | | |
| 1-(1-enyl- | (6936) S: | 0.039081 | (6753) G: | −0.03142 | 0.202913 | 8.30E−06 |
| palmitoyl)-2- | Veillonella | | Clostridium | | | |
| linoleoyl-GPE | atypica | | | | | |
| (P-16:0/18:2)* | | | | | | |
| sphingomyelin | (4670) S: | 0.04117 | (15154) F: | 0.040539 | 0.20203 | 9.11E−06 |
| (d18:2/23:0, | Coprococcus | | Clostridiales | | | |
| d18:1/23:1, | catus | | unclassified | | | |
| d17:1/24:1)* | | | | | | |
| eicosenedioate | (7061) S: | 0.028304 | (4581) S: | 0.028238 | 0.200614 | 1.05E−05 |
| (C20:1-DC)* | Lactobacillus | | Dorea | | | |
| | ruminis | | longicatena | | | |
| picolinoylglycine | (14861) U: | 0.056804 | (6939) S: | 0.046771 | 0.200551 | 1.06E−05 |
| | Unknown | | Veillonella | | | |
| | | | parvula | | | |
| 5alpha- | (4810) S: | −0.03418 | (15233) G: | −0.03312 | 0.198992 | 1.25E−05 |
| androstan- | Blautia sp | | Firmicutes | | | |
| 3alpha,17beta- | CAG 237 | | unclassified | | | |
| diol | | | | | | |
| monosulfate | | | | | | |
| (1) | | | | | | |
| S- | (15326) G: | 0.027465 | (5045) S: | −0.02684 | 0.198907 | 1.26E−05 |
| methylmethionine | Faecalibacterium | | Eubacterium | | | |
| | | | ventriosum | | | |
| glycocholate | (1786) S: | 0.047149 | (15271) S: | 0.042933 | 0.198808 | 1.27E−05 |
| glucuronide (1) | Butyricimonas | | Ruthenibacterium | | | |
| | synergistica | | lactatiformans | | | |
| 1- | (1862) S: | 0.048654 | (17256) S: | −0.04802 | 0.198734 | 1.28E−05 |
| docosahexaenoylglycerol | Bacteroides | | Bifidobacterium | | | |
| (22:6) | finegoldii | | bifidum | | | |
| dodecanedioate | (1957) S: | 0.029169 | (15154) F: | 0.022323 | 0.198302 | 1.34E−05 |
| | Bacteroides | | Clostridiales | | | |
| | sp CAG 144 | | unclassified | | | |
| androstenediol | (5082) S: | −0.03177 | (4581) S: | 0.028058 | 0.19818 | 1.35E−05 |
| (3beta,17beta) | Eubacterium | | Dorea | | | |
| monosulfate | eligens | | longicatena | | | |
| (1) | | | | | | |
| X - 16087 | (17256) S: | −0.03623 | (14823) F: | 0.028631 | 0.19514 | 1.84E−05 |
| | Bifidobacterium | | Eggerthellaceae | | | |
| | bifidum | | | | | |
| S- | (5089) S: | 0.023913 | (1872) S: | −0.02383 | 0.195008 | 1.87E−05 |
| methylcysteine | Eubacterium | | Bacteroides | | | |
| sulfoxide | sp CAG 38 | | ovatus | | | |
| X - 23314 | (15326) G: | 0.017728 | (4608) S: | −0.01673 | 0.194917 | 1.89E−05 |
| | Faecalibacterium | | Ruminococcus | | | |
| | | | torques | | | |
| N1- | (6579) S: | −0.02147 | (1784) G: | 0.020539 | 0.19484 | 1.90E−05 |
| methylinosine | Firmicutes | | Butyricimonas | | | |
| | bacterium | | | | | |
| | CAG 313 | | | | | |
| isobutyrylcarnitine | (15244) F: | 0.032305 | (14992) G: | −0.0305 | 0.194473 | 1.97E−05 |
| (C4) | Clostridiales | | Butyricicoccus | | | |
| | unclassified | | | | | |
| X - 12830 | (15049) F: | 0.027834 | (2311) F: | 0.027446 | 0.194473 | 1.97E−05 |
| | Clostridiales | | Rikenellaceae | | | |
| | unclassified | | | | | |
| pyroglutamine * | (4581) S: | 0.013744 | (15333) S: | 0.01361 | 0.193215 | 2.24E−05 |
| | Dorea | | Faecalibacterium | | | |
| | longicatena | | prausnitzii | | | |
| X - 11491 | (15154) F: | −0.02839 | (4914) S: | −0.02491 | 0.192206 | 2.47E−05 |
| | Clostridiales | | Clostridium sp | | | |
| | unclassified | | | | | |
| N-palmitoyl- | (1903) S: | 0.03977 | (15132) S: | 0.033267 | 0.192123 | 2.49E−05 |
| sphingosine | Bacteroides | | Flavonifractor | | | |
| (d18:1/16:0) | plebeius CAG | | plautii | | | |
| | 211 | | | | | |
| alpha- | (4940) S: | 0.023169 | (14909) S: | 0.023168 | 0.1913 | 2.70E−05 |
| hydroxyisocaproate | Roseburia | | Clostridium | | | |
| | inulinivorans | | sp CAG | | | |
| | | | 169 | | | |
| X - 21410 | (6783) S: | 0.057503 | (4844) S: | 0.045175 | 0.191223 | 2.72E−05 |
| | Catenibacterium | | Blautia | | | |
| | sp CAG | | obeum | | | |
| | 290 | | | | | |
| nonadecanoate | (6750) S: | 0.026632 | (15154) F: | 0.015604 | 0.191167 | 2.74E−05 |
| (19:0) | Clostridium sp | | Clostridiales | | | |
| | | | unclassified | | | |
| X - 11478 | (6340) S: | −0.03414 | (4882) S: | −0.03191 | 0.190014 | 3.07E−05 |
| | Clostridium sp | | Roseburia | | | |
| | CAG 269 | | sp CAG | | | |
| | | | 100 | | | |
| formiminoglutamate | (4714) S: | −0.03624 | (1877) S: | 0.030694 | 0.189092 | 3.36E−05 |
| | Clostridium sp | | Bacteroides | | | |
| | | | caccae | | | |
| X - 11378 | (4581) S: | 0.024786 | (6139) G: | −0.02167 | 0.188939 | 3.41E−05 |
| | Dorea | | Intestinibacter | | | |
| | longicatena | | | | | |
| erucate | (14924) S: | −0.03415 | (14992) G: | 0.033553 | 0.188356 | 3.61E−05 |
| (22:1n9) | Firmicutes | | Butyricicoccus | | | |
| | bacterium | | | | | |
| | CAG 137 | | | | | |
| 7- | (1832) S: | 0.046219 | (15154) F: | 0.032325 | 0.186213 | 4.44E−05 |
| methylxanthine | Bacteroides | | Clostridiales | | | |
| | clarus | | unclassified | | | |
| 3- | (4532) S: | 0.051178 | (1832) S: | 0.036553 | 0.185837 | 4.60E−05 |
| methylxanthine | Eubacterium | | Bacteroides | | | |
| | hallii | | clarus | | | |
| 7-alpha- | (2325) S: | −0.03875 | (4705) S: | 0.030587 | 0.185307 | 4.84E−05 |
| hydroxy-3-oxo- | Alistipes | | Clostridium | | | |
| 4-cholestenoate | indistinctus | | sp CAG | | | |
| (7-Hoca) | | | 43 | | | |
| 2- | (4933) S: | −0.03975 | (7985) S: | 0.037651 | 0.185133 | 4.92E−05 |
| aminoadipate | Eubacterium | | Lactococcus | | | |
| | rectale | | lactis | | | |
| N- | (4553) S: | 0.017304 | (5736) S: | −0.01705 | 0.184771 | 5.09E−05 |
| acetylaspartate | Clostridium sp | | Acidaminococcus | | | |
| (NAA) | | | intestini | | | |
| 3- | (14823) F: | 0.025517 | (4964) F: | 0.023516 | 0.184771 | 5.09E−05 |
| methyladipate | Eggerthellaceae | | Eubacteriaceae | | | |
| gamma- | (15286) F: | −0.02981 | (8767) U: | −0.02653 | 0.184717 | 5.12E−05 |
| glutamylleucine | Ruminococcaceae | | Unknown | | | |
| X - 12101 | (2301) S: | 0.015415 | (14992) G: | 0.01372 | 0.18465 | 5.15E−05 |
| | Alistipes | | Butyricicoccus | | | |
| | finegoldii | | | | | |
| theobromine | (14921) U: | 0.044583 | (1832) S: | 0.044038 | 0.184276 | 5.34E−05 |
| | Unknown | | Bacteroides | | | |
| | | | clarus | | | |
| 1- | (6750) S: | 0.028075 | (9226) S: | −0.0266 | 0.182811 | 6.13E−05 |
| methylhistidine | Clostridium sp | | Akkermansia | | | |
| | | | muciniphila | | | |
| trimethylamine | (4577) S: | 0.022201 | (15350) U: | 0.0202 | 0.182751 | 6.17E−05 |
| N-oxide | Coprococcus | | Unknown | | | |
| | comes | | | | | |
| X - 17654 | (9226) S: | −0.02736 | (4262) S: | 0.026172 | 0.181994 | 6.62E−05 |
| | Akkermansia | | Ruminococcus | | | |
| | muciniphila | | sp | | | |
| ximenoylcarnitine | (4828) S: | 0.026637 | (6276) S: | −0.02367 | 0.181408 | 7.00E−05 |
| (C26:1)* | Blautia sp | | Clostridium | | | |
| | | | sp CAG | | | |
| | | | 245 | | | |
| glycosyl | (4540) S: | 0.023968 | (15073) G: | 0.017841 | 0.180199 | 7.84E−05 |
| ceramide | Anaerostipes | | Oscillibacter | | | |
| (d18:2/24:1, | hadrus | | | | | |
| d18:1/24:2)* | | | | | | |
| tiglylcarnitine | (14909) S: | 0.039971 | (15332) S: | 0.031338 | 0.180062 | 7.94E−05 |
| (C5:1-DC) | Clostridium sp | | Faecalibacterium | | | |
| | CAG 169 | | prausnitzii | | | |
| isovalerylglycine | (4705) S: | −0.05482 | (4651) S: | 0.025388 | 0.179713 | 8.20E−05 |
| | Clostridium sp | | Clostridium | | | |
| | CAG 43 | | sp CAG | | | |
| | | | 230 | | | |
| glutamate | (7985) S: | 0.022629 | (4940) S: | 0.017824 | 0.179328 | 8.50E−05 |
| | Lactococcus | | Roseburia | | | |
| | lactis | | inulinivorans | | | |
| 7-methylurate | (1861) S: | −0.04538 | (5082) S: | −0.02823 | 0.179307 | 8.51E−05 |
| | Bacteroides | | Eubacterium | | | |
| | thetaiotaomicron | | eligens | | | |
| 2- | (15090) S: | 0.038815 | (4644) S: | −0.03569 | 0.179151 | 8.64E−05 |
| methylbutyrylcarnitine | Oscillibacter | | Clostridium | | | |
| (C5) | sp CAG 241 | | sp CAG | | | |
| | | | 62 | | | |
| X - 13844 | (14921) U: | 0.042185 | (6747) S: | −0.042 | 0.179028 | 8.73E−05 |
| | Unknown | | Clostridium | | | |
| | | | spiroforme | | | |
| X - 12739 | (4781) U: | −0.01699 | (14992) G: | 0.015071 | 0.178857 | 8.87E−05 |
| | Unknown | | Butyricicoccus | | | |
| androstenediol | (4882) S: | −0.0296 | (8076) S: | −0.02816 | 0.178705 | 9.00E−05 |
| (3alpha, | Roseburia sp | | Streptococcus | | | |
| 17alpha) | CAG 100 | | parasanguinis | | | |
| monosulfate | | | | | | |
| (2) | | | | | | |
| palmitoylcarnitine | (1862) S: | 0.034849 | (4940) S: | 0.033225 | 0.178654 | 9.04E−05 |
| (C16) | Bacteroides | | Roseburia | | | |
| | finegoldii | | inulinivorans | | | |
| gamma- | (4571) S: | 0.035643 | (15326) G: | −0.03152 | 0.178576 | 9.11E−05 |
| glutamyl-2- | Dorea sp CAG | | Faecalibacterium | | | |
| aminobutyrate | 105 | | | | | |
| acisoga | (4960) G: | 0.046059 | (1877) S: | 0.036021 | 0.178429 | 9.23E−05 |
| | Eubacterium | | Bacteroides | | | |
| | | | caccae | | | |
| 1-(1-enyl- | (15271) S: | 0.013828 | (4540) S: | 0.012286 | 0.177804 | 9.78E−05 |
| palmitoyl)-2- | Ruthenibacterium | | Anaerostipes | | | |
| oleoyl-GPC | lactatiformans | | hadrus | | | |
| (P-16:0/18:1)* | | | | | | |
| catechol | (14861) U: | 0.038348 | (4826) S: | −0.02748 | 0.176921 | 0.000106 |
| sulfate | Unknown | | Blautia sp | | | |
| 3- | (15369) S: | −0.01833 | (4342) U: | −0.01715 | 0.176838 | 0.000107 |
| methylcytidine | Faecalibacterium | | Unknown | | | |
| | sp CAG 74 | | | | | |
| X - 14939 | (9226) S: | −0.02471 | (6148) F: | −0.02155 | 0.176721 | 0.000108 |
| | Akkermansia | | Peptostrep- | | | |
| | muciniphila | | tococcaceae | | | |
| pregnenetriol | (4564) S: | 0.026935 | (4750) G: | 0.020832 | 0.176393 | 0.000111 |
| disulfate* | Ruminococcus | | Clostridium | | | |
| | torques | | | | | |
| 1-(1-enyl- | (5803) S: | −0.03699 | (15350) U: | 0.036086 | 0.176365 | 0.000112 |
| stearoyl)-GPE | Dialister sp | | Unknown | | | |
| (P-18:0)* | CAG 357 | | | | | |
| carnitine | (1830) S: | 0.029298 | (6347) S: | 0.025743 | 0.176034 | 0.000115 |
| | Bacteroides | | Clostridium | | | |
| | stercoris | | sp CAG | | | |
| | | | 356 | | | |
| X - 11261 | (4644) S: | −0.02467 | (4651) S: | −0.01978 | 0.175416 | 0.000122 |
| | Clostridium sp | | Clostridium | | | |
| | CAG 62 | | sp CAG | | | |
| | | | 230 | | | |
| gamma- | (4930) F: | 0.032433 | (15286) F: | 0.029908 | 0.174417 | 0.000133 |
| glutamylcitrulline* | Lachnospiraceae | | Ruminococcaceae | | | |
| N-acetyl- | (15132) S: | −0.02791 | (4804) S: | −0.0258 | 0.173841 | 0.00014 |
| isoputreanine* | Flavonifractor | | Blautia sp | | | |
| | plautii | | | | | |
| 5alpha- | (1814) S: | −0.02443 | (15216) F: | 0.017697 | 0.170402 | 0.00019 |
| pregnan- | Bacteroides | | Clostridiales | | | |
| 3beta,20alpha- | vulgatus | | unclassified | | | |
| diol | | | | | | |
| monosulfate | | | | | | |
| (2) | | | | | | |
| o-cresol sulfate | (1786) S: | 0.046263 | (4914) S: | −0.03874 | 0.169442 | 0.000207 |
| | Butyricimonas | | Clostridium sp | | | |
| | synergistica | | | | | |
| phenol | (4749) S: | 0.022088 | (15317) S: | −0.01923 | 0.169413 | 0.000208 |
| glucuronide | Clostridium sp | | Faecalibacterium | | | |
| | CAG 7 | | sp | | | |
| | | | CAG 82 | | | |
| leucine | (4564) S: | 0.027402 | (6148) F: | 0.026545 | 0.169312 | 0.00021 |
| | Ruminococcus | | Peptostrep- | | | |
| | torques | | tococcaceae | | | |
| X - 24544 | (4581) S: | 0.033527 | (15315) G: | 0.026809 | 0.169132 | 0.000213 |
| | Dorea | | Faecalibacterium | | | |
| | longicatena | | | | | |
| deoxycholate | (4552) S: | 0.047973 | (4659) S: | −0.0432 | 0.168661 | 0.000222 |
| | Ruminococcus | | Clostridium | | | |
| | sp | | sp CAG | | | |
| | | | 122 | | | |
| 2-methylserine | (2296) G: | −0.05513 | (4425) S: | −0.03964 | 0.167244 | 0.000251 |
| | Alistipes | | Ruminococcus | | | |
| | | | sp CAG | | | |
| | | | 254 | | | |
| N-stearoyl- | (4714) S: | −0.02344 | (15315) G: | −0.02246 | 0.16624 | 0.000274 |
| sphingosine | Clostridium sp | | Faecalibacterium | | | |
| (d18:1/18:0)* | | | | | | |
| 2- | (4272) S: | −0.02307 | (8010) S: | −0.02054 | 0.166116 | 0.000277 |
| aminobutyrate | Eubacterium | | Streptococcus | | | |
| | sp CAG 581 | | salivarius | | | |
| imidazole | (5089) S: | 0.02396 | (7985) S: | 0.023451 | 0.165961 | 0.00028 |
| propionate | Eubacterium | | Lactococcus | | | |
| | sp CAG 38 | | lactis | | | |
| sphingomyelin | (15266) G: | −0.023 | (14894) S: | 0.022525 | 0.165136 | 0.000301 |
| (d18:1/22:1, | Firmicutes | | Anaeroma | | | |
| d18:2/22:0, | unclassified | | ssilibacillus | | | |
| d16:1/24:1)* | | | sp An250 | | | |
| X - 16944 | (17244) S: | 0.024286 | (4816) S: | −0.02382 | 0.165071 | 0.000303 |
| | Bifidobacterium | | Blautia sp | | | |
| | adolescentis | | | | | |
| X - 24947 | (1812) S: | 0.033326 | (6750) S: | 0.022116 | 0.165035 | 0.000304 |
| | Bacteroides | | Clostridium sp | | | |
| | massiliensis | | | | | |
| indole-3- | (3926) U: | 0.021648 | (4909) G: | 0.02119 | 0.164723 | 0.000312 |
| carboxylic acid | Unknown | | Clostridium | | | |
| perfluorooctanesulfonic | (17256) S: | −0.03882 | (4557) S: | 0.038422 | 0.164055 | 0.00033 |
| acid | Bifidobacterium | | Ruminococcus | | | |
| (PFOS) | bifidum | | lactaris | | | |
| 4- | (2318) S: | −0.04684 | (15093) F: | −0.04421 | 0.162374 | 0.000381 |
| imidazoleacetate | Alistipes | | Clostridiales | | | |
| | putredinis | | unclassified | | | |
| androstenediol | (15120) S: | −0.0329 | (15233) G: | −0.03049 | 0.162146 | 0.000388 |
| (3alpha,17alpha) | Firmicutes | | Firmicutes | | | |
| monosulfate | bacterium | | unclassified | | | |
| (3) | CAG 114 | | | | | |
| X - 11444 | (4564) S: | 0.02505 | (1786) S: | 0.024484 | 0.161915 | 0.000396 |
| | Ruminococcus | | Butyricimonas | | | |
| | torques | | synergistica | | | |
| N- | (4037) S: | −0.04166 | (4564) S: | −0.03847 | 0.161896 | 0.000396 |
| methyltaurine | Clostridium | | Ruminococcus | | | |
| | innocuum | | torques | | | |
| adipoylcarnitine | (15132) S: | 0.02224 | (4940) S: | 0.021147 | 0.161523 | 0.000409 |
| (C6-DC) | Flavonifractor | | Roseburia | | | |
| | plautii | | inulinivorans | | | |
| X - 18922 | (4540) S: | −0.03347 | (6750) S: | 0.029384 | 0.161302 | 0.000417 |
| | Anaerostipes | | Clostridium sp | | | |
| | hadrus | | | | | |
| dehydroisoand | (3964) U: | −0.03037 | (8002) S: | −0.03031 | 0.160191 | 0.000457 |
| rosterone | Unknown | | Streptococcus | | | |
| sulfate (DHEA-S) | | | thermophilus | | | |
| perfluorooctanoate | (6141) F: | −0.03107 | (4933) S: | −0.02892 | 0.160113 | 0.00046 |
| (PFOA) | Peptostrep- | | Eubacterium | | | |
| | tococcaceae | | rectale | | | |
| pregn steroid | (4581) S: | 0.035894 | (4940) S: | 0.035555 | 0.159783 | 0.000473 |
| monosulfate | Dorea | | Roseburia | | | |
| C21H34O5S* | longicatena | | inulinivorans | | | |
| X - 12798 | (15340) G: | −0.03508 | (6141) F: | −0.03128 | 0.159687 | 0.000477 |
| | Faecalibacterium | | Peptostrep- | | | |
| | | | tococcaceae | | | |
| gamma- | (14993) S: | 0.021478 | (5075) S: | 0.02064 | 0.159665 | 0.000477 |
| glutamylglutamate | Butyricicoccus | | Lachnospira | | | |
| | sp | | pectinoschiza | | | |
| X - 13431 | (6148) F: | 0.034411 | (5121) S: | 0.032508 | 0.159559 | 0.000482 |
| | Peptostrep- | | Clostridium | | | |
| | tococcaceae | | sp CAG | | | |
| | | | 264 | | | |
| caffeic acid | (14861) U: | 0.026993 | (3957) F: | −0.02187 | 0.159463 | 0.000486 |
| sulfate | Unknown | | Lachnospiraceae | | | |
| 4- | (4121) U: | 0.026234 | (4933) S: | −0.02563 | 0.159245 | 0.000494 |
| hydroxychlorothalonil | Unknown | | Eubacterium | | | |
| | | | rectale | | | |
| X - 17685 | (4447) S: | −0.04234 | (4705) S: | −0.03739 | 0.1588 | 0.000513 |
| | Eubacterium | | Clostridium | | | |
| | sp CAG 274 | | sp CAG | | | |
| | | | 43 | | | |
| thyroxine | (4811) S: | 0.021084 | (5792) S: | −0.01971 | 0.158642 | 0.00052 |
| | Blautia | | Phascolarctobacterium | | | |
| | obeum | | sp CAG | | | |
| | | | 207 | | | |
| sphingomyelin | (15333) S: | −0.02757 | (14909) S: | −0.02458 | 0.158003 | 0.000548 |
| (d18:2/24:1, | Faecalibacterium | | Clostridium | | | |
| d18:1/24:2)* | prausnitzii | | sp CAG | | | |
| | | | 169 | | | |
| Fibrinopeptide | (1786) S: | −0.03762 | (5045) S: | −0.03299 | 0.157893 | 0.000553 |
| A (3-16)** | Butyricimonas | | Eubacterium | | | |
| | synergistica | | ventriosum | | | |
| pregnanediol- | (4644) S: | 0.027471 | (4532) S: | −0.02173 | 0.157596 | 0.000566 |
| 3-glucuronide | Clostridium sp | | Eubacterium | | | |
| | CAG 62 | | hallii | | | |
| N- | (4933) S: | −0.02409 | (4571) S: | 0.022616 | 0.156582 | 0.000615 |
| acetylarginine | Eubacterium | | Dorea sp | | | |
| | rectale | | CAG 105 | | | |
| pregnen-diol | (4940) S: | 0.029751 | (6328) S: | −0.02762 | 0.15617 | 0.000636 |
| disulfate | Roseburia | | Clostridium | | | |
| C21H34O8S2* | inulinivorans | | sp CAG | | | |
| | | | 492 | | | |
| 1-oleoyl-2- | (5736) S: | −0.0273 | (4540) S: | 0.024308 | 0.156064 | 0.000642 |
| docosahexaenoyl- | Acidaminococcus | | Anaerostipes | | | |
| GPC | intestini | | hadrus | | | |
| (18:1/22:6)* | | | | | | |
| 3-(4- | (4581) S: | 0.03186 | (14899) U: | 0.030331 | 0.155544 | 0.000669 |
| hydroxyphenyl)lactate | Dorea | | Unknown | | | |
| | longicatena | | | | | |
| N-acetylglycine | (8601) S: | 0.037245 | (4816) S: | 0.036725 | 0.155199 | 0.000688 |
| | Candidatus | | Blautia sp | | | |
| | Gastranaerophilales | | | | | |
| | bacterium | | | | | |
| | HUM 10 | | | | | |
| propionylglycine | (4447) S: | −0.02902 | (1626) S: | 0.028992 | 0.15484 | 0.000709 |
| | Eubacterium | | Prevotellacopri | | | |
| | sp CAG 274 | | | | | |
| taurine | (14894) S: | 0.017189 | (6173) S: | 0.014888 | 0.154013 | 0.000757 |
| | Anaeromassili | | Clostridium | | | |
| | bacillus sp | | sp CAG | | | |
| | An250 | | 221 | | | |
| glycine | (15216) F: | −0.03834 | (7061) S: | 0.036169 | 0.153227 | 0.000807 |
| conjugate of | Clostridiales | | Lactobacillus | | | |
| C10H14O2 (1)* | unclassified | | ruminis | | | |
| sphingomyelin | (15373) F: | 0.027889 | (1934) S: | 0.026974 | 0.153218 | 0.000807 |
| (d18:1/21:0, | Ruminococcaceae | | Parabacteroides | | | |
| d17:1/22:0, | | | distasonis | | | |
| d16:1/23:0)* | | | | | | |
| acetylcarnitine | (1812) S: | 0.019671 | (6750) S: | 0.019283 | 0.152854 | 0.000831 |
| (C2) | Bacteroides | | Clostridium sp | | | |
| | massiliensis | | | | | |
| X - 18899 | (14992) G: | 0.015723 | (14993) S: | 0.015681 | 0.152617 | 0.000847 |
| | Butyricicoccus | | Butyricicoccus sp | | | |
| X - 12906 | (4930) F: | 0.039781 | (6376) F: | 0.037775 | 0.152538 | 0.000852 |
| | Lachnospiraceae | | Clostridiaceae | | | |
| 3-sulfo-L- | (15385) U: | 0.033842 | (4670) S: | −0.03222 | 0.152176 | 0.000877 |
| alanine | Unknown | | Coprococcus | | | |
| | | | catus | | | |
| biliverdin | (15286) F: | 0.02333 | (14844) S: | −0.01748 | 0.152148 | 0.000879 |
| | Ruminococcaceae | | Firmicutes | | | |
| | | | bacterium | | | |
| | | | CAG 94 | | | |
| 1-linoleoyl- | (1786) S: | 0.023611 | (14400) G: | −0.01513 | 0.151872 | 0.000898 |
| GPA (18:2)* | Butyricimonas | | Collinsella | | | |
| | synergistica | | | | | |
| 3-hydroxy-2- | (15452) S: | 0.025741 | (4532) S: | −0.02396 | 0.151481 | 0.000927 |
| ethylpropionate | Bilophila sp 4 | | Eubacterium | | | |
| | 1 30 | | hallii | | | |
| carotene diol | (8002) S: | −0.0236 | (4810) S: | 0.019618 | 0.151433 | 0.00093 |
| (3) | Streptococcus | | Blautia sp | | | |
| | thermophilus | | CAG 237 | | | |
| X - 17325 | (15154) F: | 0.019388 | (4940) S: | −0.019 | 0.149107 | 0.001117 |
| | Clostridiales | | Roseburia | | | |
| | unclassified | | inulinivorans | | | |
| docosahexaenoate | (15295) G: | 0.017696 | (3957) F: | −0.01692 | 0.148078 | 0.001209 |
| (DHA; | Gemmiger | | Lachnospiraceae | | | |
| 22:6n3) | | | | | | |
| N6,N6,N6- | (2318) S: | −0.02004 | (15332) S: | 0.019503 | 0.147817 | 0.001234 |
| trimethyllysine | Alistipes | | Faecalibacterium | | | |
| | putredinis | | prausnitzii | | | |
| deoxycarnitine | (15346) G: | 0.030403 | (5083) G: | 0.028617 | 0.147739 | 0.001242 |
| | Faecalibacterium | | Eubacterium | | | |
| 2,3-dihydroxy- | (15216) F: | −0.02283 | (1877) S: | 0.0228 | 0.147388 | 0.001276 |
| 5-methylthio- | Clostridiales | | Bacteroides | | | |
| 4-pentenoate | unclassified | | caccae | | | |
| (DMTPA)* | | | | | | |
| arabonate/xylonate | (4961) G: | 0.022231 | (4608) S: | −0.02199 | 0.146798 | 0.001335 |
| | Eubacterium | | Ruminococcus | | | |
| | | | torques | | | |
| X - 11852 | (4893) S: | 0.017806 | (4957) F: | 0.01425 | 0.146358 | 0.001381 |
| | Clostridium sp | | Eubacteriaceae | | | |
| urea | (15078) S: | −0.03091 | (5190) S: | 0.02188 | 0.146356 | 0.001381 |
| | Oscillibacter | | Firmicutes | | | |
| | sp | | bacterium | | | |
| | | | CAG 102 | | | |
| indoleacetylglutamine | (4447) S: | −0.03378 | (4749) S: | −0.031 | 0.145985 | 0.001421 |
| | Eubacterium | | Clostridium | | | |
| | sp CAG 274 | | sp CAG | | | |
| | | | 7 | | | |
| vanillylmandelate | (9262) S: | −0.01662 | (15318) S: | 0.013687 | 0.145053 | 0.001525 |
| (VMA) | Burkholderiales | | Faecalibacterium | | | |
| | bacterium | | prausnitzii | | | |
| | 1 1 47 | | | | | |
| X - 13255 | (17239) S: | 0.046643 | (4961) G: | 0.042712 | 0.144663 | 0.001571 |
| | Bifidobacterium | | Eubacterium | | | |
| | sp N4G05 | | | | | |
| androstenediol | (4564) S: | 0.021734 | (4782) U: | −0.02155 | 0.1441 | 0.00164 |
| (3beta,17beta) | Ruminococcus | | Unknown | | | |
| disulfate (1) | torques | | | | | |
| valine | (6179) G: | 0.031487 | (7985) S: | 0.030513 | 0.143921 | 0.001662 |
| | Clostridium | | Lactococcus | | | |
| | | | lactis | | | |
| X - 11485 | (1786) S: | 0.034559 | (4553) S: | 0.032995 | 0.143508 | 0.001715 |
| | Butyricimonas | | Clostridium sp | | | |
| | synergistica | | | | | |
| X - 24757 | (15085) F: | 0.020657 | (4909) G: | 0.019259 | 0.143247 | 0.001749 |
| | Clostridiales | | Clostridium | | | |
| | unclassified | | | | | |
| chenodeoxycholate | (4552) S: | −0.03176 | (2301) S: | −0.02344 | 0.143007 | 0.00178 |
| | Ruminococcus | | Alistipes | | | |
| | sp | | finegoldii | | | |
| 17- | (15073) G: | 0.0211 | (4940) S: | 0.020984 | 0.142649 | 0.001829 |
| methylstearate | Oscillibacter | | Roseburia | | | |
| | | | inulinivorans | | | |
| 3- | (14400) G: | −0.04178 | (14974) U: | 0.036425 | 0.142441 | 0.001857 |
| hydroxybutyryl | Collinsella | | Unknown | | | |
| carnitine (1) | | | | | | |
| sphingomyelin | (15452) S: | −0.01411 | (4648) G: | 0.013313 | 0.1424 | 0.001863 |
| (d18:2/24:2)* | Bilophila sp 4 | | Roseburia | | | |
| | 1 30 | | | | | |
| 5alpha- | (15346) G: | 0.026787 | (15120) S: | −0.02311 | 0.142374 | 0.001867 |
| androstan- | Faecalibacterium | | Firmicutes | | | |
| 3beta,17beta- | | | bacterium | | | |
| diol | | | CAG 114 | | | |
| monosulfate | | | | | | |
| (2) | | | | | | |
| stearoyl | (15317) S: | −0.03088 | (5082) S: | 0.029658 | 0.142261 | 0.001883 |
| sphingomyelin | Faecalibacterium | | Eubacterium | | | |
| (d18:1/18:0) | sp CAG 82 | | eligens | | | |
| 2- | (14963) S: | −0.02409 | (4828) S: | 0.020904 | 0.142222 | 0.001888 |
| linoleoylglycerol | Anaerotruncus | | Blautia sp | | | |
| (18:2) | colihominis | | | | | |
| xanthurenate | (17237) S: | −0.02659 | (6179) G: | 0.025405 | 0.142175 | 0.001895 |
| | Bifidobacterium | | Clostridium | | | |
| | pseudocatenulatum | | | | | |
| X - 12411 | (14807) S: | −0.02183 | (1836) S: | −0.02029 | 0.142173 | 0.001895 |
| | Gordonibacter | | Bacteroides | | | |
| | pamelaeae | | uniformis | | | |
| 5-oxoproline | (15081) F: | 0.019373 | (6179) G: | 0.016099 | 0.142122 | 0.001902 |
| | Clostridiales | | Clostridium | | | |
| | unclassified | | | | | |
| 1-(1-enyl- | (4721) S: | 0.014673 | (4705) S: | −0.01447 | 0.141822 | 0.001945 |
| palmitoyl)-GPC | Clostridium sp | | Clostridium | | | |
| (P-16:0)* | CAG 58 | | sp CAG | | | |
| | | | 43 | | | |
| N- | (4844) S: | −0.02196 | (14974) U: | 0.02064 | 0.14181 | 0.001947 |
| acetylglutamate | Blautia | | Unknown | | | |
| | obeum | | | | | |
| tetradecanedioate | (4930) F: | −0.05114 | (4914) S: | −0.0502 | 0.141803 | 0.001948 |
| | Lachnospiraceae | | Clostridium sp | | | |
| glutarylcarnitine | (4820) S: | −0.01929 | (1949) S: | 0.018906 | 0.141384 | 0.00201 |
| (C5-DC) | Blautia sp | | Parabacteroides | | | |
| | | | merdae | | | |
| X - 24337 | (15272) F: | −0.02787 | (4816) S: | −0.02418 | 0.140613 | 0.002128 |
| | Ruminococcaceae | | Blautia sp | | | |
| gamma- | (15286) F: | −0.02942 | (15332) S: | 0.029158 | 0.140431 | 0.002157 |
| glutamylisoleucine* | Ruminococcaceae | | Faecalibacterium | | | |
| | | | prausnitzii | | | |
| 1-(1-enyl- | (6148) F: | 0.03825 | (4874) S: | 0.029863 | 0.140159 | 0.0022 |
| palmitoyl)-2- | Peptostrep- | | Fusicatenibacter | | | |
| arachidonoyl- | tococcaceae | | saccharivorans | | | |
| GPC (P- | | | | | | |
| 16:0/20:4)* | | | | | | |
| 1-(1-enyl- | (2311) F: | 0.032348 | (5190) S: | 0.031149 | 0.140091 | 0.002211 |
| stearoyl)-2- | Rikenellaceae | | Firmicutes | | | |
| oleoyl-GPE | | | bacterium | | | |
| (P-18:0/18:1) | | | CAG 102 | | | |
| 1-(1-enyl- | (4782) U: | −0.02249 | (4721) S: | 0.019978 | 0.13999 | 0.002228 |
| palmitoyl)-GPE | Unknown | | Clostridium | | | |
| (P-16:0)* | | | sp CAG | | | |
| | | | 58 | | | |
| epiandrosterone | (15260) G: | 0.026216 | (14470) G: | 0.021722 | 0.139978 | 0.00223 |
| sulfate | Firmicutes | | Collinsella | | | |
| | unclassified | | | | | |
| 2- | (17241) S: | 0.032773 | (15143) S: | 0.010676 | 0.139865 | 0.002249 |
| acetamidophenol | Bifidobacterium | | Flavonifractor sp | | | |
| sulfate | catenulatum | | | | | |
| 1-myristoyl-2- | (1832) S: | −0.01363 | (9226) S: | 0.011531 | 0.139792 | 0.00226 |
| arachidonoyl- | Bacteroides | | Akkermansia | | | |
| GPC | clarus | | muciniphila | | | |
| (14:0/20:4)* | | | | | | |
| N,N,N- | (6750) S: | 0.030556 | (1785) S: | 0.025528 | 0.139762 | 0.002266 |
| trimethyl- | Clostridium sp | | Butyricimonas sp | | | |
| alanylproline | | | An62 | | | |
| betaine | | | | | | |
| (TMAP) | | | | | | |
| X - 13684 | (15390) U: | −0.01587 | (15224) F: | −0.01533 | 0.139271 | 0.002349 |
| | Unknown | | Clostridiales | | | |
| | | | unclassified | | | |
| X - 24748 | (6754) S: | 0.017642 | (15271) S: | −0.01679 | 0.138689 | 0.002451 |
| | Clostridium sp | | Ruthenibacterium | | | |
| | | | lactatiformans | | | |
| malate | (4938) S: | 0.017703 | (15073) G: | 0.015187 | 0.138301 | 0.002521 |
| | Roseburia sp | | Oscillibacter | | | |
| isovalerylcarnitine | (4121) U: | 0.03584 | (15332) S: | 0.034336 | 0.138116 | 0.002555 |
| (C5) | Unknown | | Faecalibacterium | | | |
| | | | prausnitzii | | | |
| 2- | (17249) S: | −0.03766 | (14992) G: | 0.035068 | 0.137906 | 0.002595 |
| hydroxynervonate* | Bifidobacterium | | Butyricicoccus | | | |
| | longum | | | | | |
| X - 11858 | (4582) S: | −0.01276 | (6347) S: | 0.01219 | 0.137828 | 0.002609 |
| | Dorea | | Clostridium | | | |
| | longicatena | | sp CAG | | | |
| | | | 356 | | | |
| 3- | (15085) F: | 0.031154 | (14992) G: | 0.022437 | 0.136824 | 0.002806 |
| hydroxyhippurate | Clostridiales | | Butyricicoccus | | | |
| sulfate | unclassified | | | | | |
| lactosyl-N- | (5843) S: | −0.03117 | (14322) S: | 0.017928 | 0.136052 | 0.002966 |
| nervonoyl- | Allisonella | | Eggerthella | | | |
| sphingosine | histaminiformans | | sp CAG | | | |
| (d18:1/24:1)* | | | 209 | | | |
| 1-(1-enyl- | (6936) S: | 0.015436 | (4659) S: | 0.01529 | 0.135874 | 0.003005 |
| palmitoyl)-2- | Veillonella | | Clostridium | | | |
| oleoyl-GPE | atypica | | sp CAG | | | |
| (P-16:0/18:1)* | | | 122 | | | |
| X - 18886 | (4810) S: | −0.02418 | (14594) G: | 0.02417 | 0.135841 | 0.003012 |
| | Blautia sp | | Collinsella | | | |
| | CAG 237 | | | | | |
| Fibrinopeptide | (1949) S: | −0.01591 | (15317) S: | −0.01575 | 0.135774 | 0.003026 |
| B (1-13)** | Parabacteroides | | Faecalibacterium sp | | | |
| | merdae | | CAG 82 | | | |
| taurochenodeoxycholic | (4664) S: | 0.007057 | (4557) S: | 0.006881 | 0.134882 | 0.003225 |
| acid 3- | Roseburia sp | | Ruminococcus | | | |
| sulfate | CAG 303 | | lactaris | | | |
| DSGEGDFXAEGGGVR* | (2303) S: | −0.0239 | (9391) F: | −0.0223 | 0.134124 | 0.003404 |
| | Alistipes | | Oxalobacteraceae | | | |
| | finegoldii | | | | | |
| tauroursodeoxycholate | (14341) S: | −0.02604 | (1867) S: | 0.025111 | 0.133987 | 0.003437 |
| | Eggerthella sp | | Bacteroides | | | |
| | CAG 298 | | xylanisolvens | | | |
| X - 13723 | (4261) G: | 0.033265 | (4868) S: | −0.02808 | 0.133722 | 0.003502 |
| | Blautia | | Blautia sp | | | |
| 1-stearoyl-2- | (15385) U: | −0.0234 | (4810) S: | 0.021333 | 0.133381 | 0.003587 |
| docosahexaenoyl-GPE | Unknown | | Blautia sp | | | |
| (18:0/22:6)* | | | CAG 237 | | | |
| 14-HDoHE/17- | (15460) F: | 0.026875 | (1784) G: | 0.02536 | 0.133132 | 0.003651 |
| HDoHE | Desulfovibrionaceae | | Butyricimonas | | | |
| 1- | (6962) S: | 0.008273 | (4871) S: | 0.00802 | 0.13212 | 0.00392 |
| linolenoylglycerol | Megamonas | | Ruminococcus | | | |
| (18:3) | funiformis | | sp | | | |
| X - 11299 | (4553) S: | 0.024483 | (15385) U: | 0.022957 | 0.131227 | 0.004172 |
| | Clostridium sp | | Unknown | | | |
| X - 21285 | (9283) S: | 0.037269 | (15350) U: | 0.032192 | 0.130566 | 0.004367 |
| | Sutterella | | Unknown | | | |
| | wadsworthensis | | | | | |
| Fibrinopeptide | (1786) S: | −0.03017 | (5045) S: | −0.02522 | 0.129638 | 0.004656 |
| A (5-16)* | Butyricimonas | | Eubacterium | | | |
| | synergistica | | ventriosum | | | |
| X - 21661 | (4811) S: | −0.01344 | (14594) G: | −0.00963 | 0.129284 | 0.004771 |
| | Blautia | | Collinsella | | | |
| | obeum | | | | | |
| dodecenedioate | (14114) S: | 0.031723 | (6148) F: | −0.02984 | 0.128831 | 0.004921 |
| (C12:1-DC)* | Subdoligranulum | | Peptostrep- | | | |
| | sp CAG | | tococcaceae | | | |
| | 314 | | | | | |
| 3-methyl-2- | (4706) F: | 0.019187 | (15390) U: | −0.01839 | 0.128595 | 0.005001 |
| oxovalerate | Clostridiaceae | | Unknown | | | |
| X - 11847 | (15271) S: | −0.0142 | (4582) S: | −0.01128 | 0.128021 | 0.005201 |
| | Ruthenibacterium | | Dorea | | | |
| | lactatiformans | | longicatena | | | |
| 1-myristoyl-2- | (6338) F: | −0.01141 | (1962) S: | 0.011219 | 0.127609 | 0.005349 |
| palmitoyl-GPC | Clostridiaceae | | Coprobacter | | | |
| (14:0/16:0) | | | secundus | | | |
| 3-aminoisobutyrate | (15124) F: | −0.024 | (15271) S: | −0.02275 | 0.127528 | 0.005378 |
| | Clostridiales | | Ruthenibacterium | | | |
| | unclassified | | lactatiformans | | | |
| stachydrine | (4961) G: | 0.022511 | (14999) U: | −0.01805 | 0.127415 | 0.00542 |
| | Eubacterium | | Unknown | | | |
| eicosenoate | (3957) F: | −0.01721 | (5075) S: | 0.016961 | 0.127302 | 0.005461 |
| (20:1) | Lachnospiraceae | | Lachnospira | | | |
| | | | pectinoschiza | | | |
| isocitrate | (4938) S: | −0.02327 | (15326) G: | 0.017973 | 0.1267 | 0.005688 |
| | Roseburia sp | | Faecalibacterium | | | |
| X - 21364 | (8076) S: | −0.01727 | (4951) S: | 0.015285 | 0.126682 | 0.005695 |
| | Streptococcus | | Roseburia | | | |
| | parasanguinis | | intestinalis | | | |
| X - 12007 | (15254) F: | 0.021132 | (9333) S: | −0.01973 | 0.126616 | 0.00572 |
| | Clostridiales | | Acetobacter | | | |
| | unclassified | | sp CAG | | | |
| | | | 267 | | | |
| N1-Methyl-2- | (8002) S: | 0.030264 | (5803) S: | −0.02372 | 0.126496 | 0.005767 |
| pyridone-5- | Streptococcus | | Dialister sp | | | |
| carboxamide | thermophilus | | CAG 357 | | | |
| X - 21659 | (4939) G: | 0.036695 | (4953) S: | 0.03646 | 0.126145 | 0.005905 |
| | Roseburia | | Roseburia | | | |
| | | | sp CAG | | | |
| | | | 182 | | | |
| gamma- | (4716) S: | 0.019939 | (1934) S: | −0.01591 | 0.126038 | 0.005947 |
| tocopherol/beta- | Clostridium sp | | Parabacteroides | | | |
| tocopherol | | | distasonis | | | |
| X - 12117 | (1836) S: | −0.02601 | (4644) S: | −0.02354 | 0.125916 | 0.005996 |
| | Bacteroides | | Clostridium | | | |
| | uniformis | | sp CAG | | | |
| | | | 62 | | | |
| 1- | (1790) S: | −0.02251 | (1862) S: | 0.021676 | 0.125693 | 0.006086 |
| myristoylglycerol | Odoribacter | | Bacteroides | | | |
| (14:0) | splanchnicus | | finegoldii | | | |
| X - 21845 | (15265) S: | 0.028019 | (14797) G: | 0.023629 | 0.125549 | 0.006145 |
| | Firmicutes | | Adlercreutzia | | | |
| | bacterium | | | | | |
| | CAG 103 | | | | | |
| N- | (14992) G: | 0.024679 | (4582) S: | −0.02289 | 0.125506 | 0.006163 |
| methylhydroxy | Butyricicoccus | | Dorea | | | |
| proline** | | | longicatena | | | |
| stearoylcarnitine | (17249) S: | −0.02445 | (1812) S: | 0.022237 | 0.125349 | 0.006228 |
| (C18) | Bifidobacterium | | Bacteroides | | | |
| | longum | | massiliensis | | | |
| X - 24546 | (4940) S: | 0.035016 | (4750) G: | 0.032653 | 0.125194 | 0.006293 |
| | Roseburia | | Clostridium | | | |
| | inulinivorans | | | | | |
| 2- | (15154) F: | 0.014856 | (3989) F: | 0.012342 | 0.124995 | 0.006377 |
| hydroxyglutarate | Clostridiales | | Firmicutes | | | |
| | unclassified | | unclassified | | | |
| X - 23787 | (15317) S: | 0.019888 | (1836) S: | 0.019399 | 0.124908 | 0.006414 |
| | Faecalibacterium | | Bacteroides | | | |
| | sp CAG 82 | | uniformis | | | |
| 4- | (14322) S: | 0.027768 | (5803) S: | 0.026381 | 0.124668 | 0.006518 |
| hydroxyhippurate | Eggerthella sp | | Dialister sp | | | |
| | CAG 209 | | CAG 357 | | | |
| glycylvaline | (1963) S: | 0.022131 | (14894) S: | 0.020056 | 0.124352 | 0.006656 |
| | Coprobacter | | Anaeroma | | | |
| | fastidiosus | | ssilibacillus | | | |
| | | | sp An250 | | | |
| cerotoylcarnitine | (15346) G: | 0.019902 | (17237) S: | −0.01903 | 0.124253 | 0.0067 |
| (C26)* | Faecalibacterium | | Bifidobacterium | | | |
| | | | pseudocatenulatum | | | |
| methylsuccinoylcarnitine | (1965) S: | 0.015991 | (4261) G: | 0.012935 | 0.123073 | 0.007243 |
| (1) | Bacteroides | | Blautia | | | |
| | sp CAG 20 | | | | | |
| X - 15492 | (6367) F: | −0.03864 | (4940) S: | 0.024465 | 0.123061 | 0.007248 |
| | Clostridiaceae | | Roseburia | | | |
| | | | inulinivorans | | | |
| X - 23585 | (9262) S: | 0.031296 | (14861) U: | 0.021237 | 0.122612 | 0.007465 |
| | Burkholderiales | | Unknown | | | |
| | bacterium | | | | | |
| | 1 1 47 | | | | | |
| X - 24556 | (5082) S: | 0.02246 | (15318) S: | 0.021364 | 0.120816 | 0.008393 |
| | Eubacterium | | Faecalibacterium | | | |
| | eligens | | prausnitzii | | | |
| N1- | (1790) S: | −0.01772 | (4705) S: | 0.012148 | 0.120422 | 0.008609 |
| methyladenosine | Odoribacter | | Clostridium | | | |
| | splanchnicus | | sp CAG | | | |
| | | | 43 | | | |
| 1,2,3- | (17244) S: | −0.02127 | (1830) S: | −0.0201 | 0.120351 | 0.008649 |
| benzenetriol | Bifidobacterium | | Bacteroides | | | |
| sulfate (2) | adolescentis | | stercoris | | | |
| 21- | (6359) F: | −0.03473 | (4564) S: | 0.032184 | 0.119965 | 0.008867 |
| hydroxypregnenolone | Clostridiaceae | | Ruminococcus | | | |
| disulfate | | | torques | | | |
| hexanoylglutamine | (14992) G: | 0.036528 | (4874) S: | −0.03028 | 0.119813 | 0.008954 |
| | Butyricicoccus | | Fusicatenibacter | | | |
| | | | saccharivorans | | | |
| X - 17367 | (15154) F: | 0.012108 | (4537) S: | −0.01195 | 0.119767 | 0.008981 |
| | Clostridiales | | Eubacterium | | | |
| | unclassified | | hallii | | | |
| tridecenedioate | (15132) S: | 0.045078 | (6174) S: | 0.044543 | 0.119127 | 0.009357 |
| (C13:1-DC)* | Flavonifractor | | Clostridium | | | |
| | plautii | | sp CAG | | | |
| | | | 265 | | | |
| phytanate | (15073) G: | 0.017338 | (14823) F: | 0.017013 | 0.118948 | 0.009464 |
| | Oscillibacter | | Eggerthellaceae | | | |
| hydroxy- | (14263) U: | −0.03089 | (15295) G: | 0.027677 | 0.11774 | 0.010221 |
| CMPF* | Unknown | | Gemmiger | | | |
| N-palmitoyl- | (15146) F: | 0.0221 | (15216) F: | −0.02184 | 0.117704 | 0.010244 |
| sphinganine | Clostridiales | | Clostridiales | | | |
| (d18:0/16:0) | unclassified | | unclassified | | | |
| 4-methyl-2- | (6148) F: | 0.02125 | (4648) G: | −0.01851 | 0.11718 | 0.01059 |
| oxopentanoate | Peptostrep- | | Roseburia | | | |
| | tococcaceae | | | | | |
| cys-gly, | (4303) S: | 0.02136 | (4820) S: | −0.02018 | 0.117156 | 0.010606 |
| oxidized | Clostridium sp | | Blautia sp | | | |
| | CAG 217 | | | | | |
| glycerate | (14313) S: | −0.02306 | (4834) G: | 0.019943 | 0.117141 | 0.010616 |
| | Clostridium sp | | Blautia | | | |
| | CAG 226 | | | | | |
| bradykinin, | (4959) S: | 0.009207 | (4811) S: | 0.007451 | 0.116402 | 0.011121 |
| des-arg(9) | Eubacterium | | Blautia | | | |
| | ramulus | | obeum | | | |
| 15- | (4714) S: | −0.01907 | (15350) U: | 0.018079 | 0.116125 | 0.011316 |
| methylpalmitate | Clostridium sp | | Unknown | | | |
| X - 11795 | (15124) F: | −0.0347 | (5803) S: | 0.025284 | 0.116105 | 0.01133 |
| | Clostridiales | | Dialister sp | | | |
| | unclassified | | CAG 357 | | | |
| 16a-hydroxy | (4782) U: | −0.0193 | (4564) S: | 0.018405 | 0.115506 | 0.011762 |
| DHEA 3-sulfate | Unknown | | Ruminococcus | | | |
| | | | torques | | | |
| arachidoylcarnitine | (4933) S: | −0.05449 | (15451) G: | −0.03232 | 0.115399 | 0.011841 |
| (C20)* | Eubacterium | | Bilophila | | | |
| | rectale | | | | | |
| choline | (15081) F: | 0.013512 | (5087) S: | 0.013325 | 0.115075 | 0.012082 |
| | Clostridiales | | Eubacterium | | | |
| | unclassified | | sp CAG | | | |
| | | | 86 | | | |
| palmitoyl | (4540) S: | 0.021119 | (4670) S: | 0.019749 | 0.114709 | 0.01236 |
| dihydrosphingomyelin | Anaerostipes | | Coprococcus | | | |
| (d18:0/16:0)* | hadrus | | catus | | | |
| glycosyl-N- | (5843) S: | −0.02223 | (15073) G: | 0.013519 | 0.114454 | 0.012557 |
| behenoyl- | Allisonella | | Oscillibacter | | | |
| sphingadienine | histaminiformans | | | | | |
| (d18:2/22:0)* | | | | | | |
| hydroxy- | (4564) S: | 0.016937 | (15346) G: | 0.014591 | 0.11419 | 0.012763 |
| N6,N6,N6- | Ruminococcus | | Faecalibacterium | | | |
| trimethyllysine * | torques | | | | | |
| lysine | (8002) S: | 0.028247 | (1830) S: | 0.027797 | 0.114182 | 0.012769 |
| | Streptococcus | | Bacteroides | | | |
| | thermophilus | | stercoris | | | |
| tyrosine | (9298) F: | −0.02194 | (7044) S: | 0.021725 | 0.114134 | 0.012808 |
| | Sutterellaceae | | Lactobacillus | | | |
| | | | acidophilus | | | |
| androsterone | (15233) G: | −0.02419 | (8002) S: | −0.02177 | 0.113555 | 0.013273 |
| sulfate | Firmicutes | | Streptococcus | | | |
| | unclassified | | thermophilus | | | |
| glycodeoxycholate | (5121) S: | −0.02394 | (15078) S: | 0.021689 | 0.113258 | 0.013517 |
| sulfate | Clostridium sp | | Oscillibacter sp | | | |
| | CAG 264 | | | | | |
| alpha- | (17244) S: | −0.02156 | (15452) S: | 0.018676 | 0.113166 | 0.013593 |
| tocopherol | Bifidobacterium | | Bilophila | | | |
| | adolescentis | | sp 4 1 30 | | | |
| 3-(3- | (14823) F: | 0.016505 | (1872) S: | −0.01511 | 0.112805 | 0.013898 |
| hydroxyphenyl)propionate | Eggerthellaceae | | Bacteroides | | | |
| sulfate | | | ovatus | | | |
| linoleate | (4936) S: | 0.016353 | (5111) S: | 0.015685 | 0.112626 | 0.01405 |
| (18:2n6) | Roseburia | | Clostridium | | | |
| | hominis | | sp CAG | | | |
| | | | 127 | | | |
| 17alpha- | (5190) S: | −0.02901 | (4781) U: | −0.0244 | 0.111788 | 0.014786 |
| hydroxypregnenolone 3- | Firmicutes | | Unknown | | | |
| sulfate | bacterium | | | | | |
| | CAG 102 | | | | | |
| xanthosine | (6939) S: | 0.015029 | (4571) S: | 0.013364 | 0.111532 | 0.015017 |
| | Veillonella | | Dorea sp | | | |
| | parvula | | CAG 105 | | | |
| 4- | (4868) S: | 0.024994 | (4844) S: | −0.02202 | 0.111379 | 0.015156 |
| hydroxyphenyl | Blautia sp | | Blautia | | | |
| pyruvate | | | obeum | | | |
| S- | (4957) F: | 0.018829 | (8002) S: | −0.01823 | 0.110711 | 0.01578 |
| methylcysteine | Eubacteriaceae | | Streptococcus | | | |
| | | | thermophilus | | | |
| dodecadienoate | (4936) S: | 0.007497 | (5792) S: | 0.006512 | 0.110622 | 0.015865 |
| (12:2)* | Roseburia | | Phascolarctobacterium | | | |
| | hominis | | sp CAG | | | |
| | | | 207 | | | |
| 1-palmitoyl-2- | (5190) S: | −0.01815 | (1962) S: | 0.011785 | 0.110232 | 0.016241 |
| palmitoleoyl- | Firmicutes | | Coprobacter | | | |
| GPC | bacterium | | secundus | | | |
| (16:0/16:1)* | CAG 102 | | | | | |
| 2- | (1862) S: | 0.024847 | (6962) S: | 0.021436 | 0.109731 | 0.016736 |
| arachidonoylglycerol | Bacteroides | | Megamonas | | | |
| (20:4) | finegoldii | | funiformis | | | |
| sphingomyelin | (4782) U: | −0.02675 | (14921) U: | 0.021965 | 0.109634 | 0.016833 |
| (d18:1/25:0, | Unknown | | Unknown | | | |
| d19:0/24:1, | | | | | | |
| d20:1/23:0, | | | | | | |
| d19:1/24:0)* | | | | | | |
| 1-palmitoyl-2- | (15124) F: | 0.017688 | (15225) F: | −0.0144 | 0.10905 | 0.017429 |
| docosahexaenoyl- | Clostridiales | | Clostridiales | | | |
| GPC | unclassified | | unclassified | | | |
| (16:0/22:6) | | | | | | |
| Fibrinopeptide | (9391) F: | −0.02671 | (4782) U: | −0.02297 | 0.108842 | 0.017646 |
| A (7-16)* | Oxalobacteraceae | | Unknown | | | |
| N6- | (15342) S: | 0.00907 | (15216) F: | −0.00651 | 0.108627 | 0.017873 |
| carbamoylthre- | Faecalibacterium | | Clostridiales | | | |
| onyladenosine | prausnitzii | | unclassified | | | |
| glycohyocholate | (15265) S: | −0.04384 | (15342) S: | 0.03827 | 0.108537 | 0.017968 |
| | Firmicutes | | Faecalibacterium | | | |
| | bacterium | | prausnitzii | | | |
| | CAG 103 | | | | | |
| N- | (1867) S: | 0.029494 | (17249) S: | −0.02421 | 0.108424 | 0.018088 |
| oleoyltaurine | Bacteroides | | Bifidobacterium | | | |
| | xylanisolvens | | longum | | | |
| X - 11593 | (4886) S: | 0.012074 | (4658) S: | 0.009798 | 0.108193 | 0.018337 |
| | Firmicutes | | Clostridium | | | |
| | bacterium | | sp CAG | | | |
| | CAG 194 | | 253 | | | |
| phenyllactate | (4925) S: | 0.022219 | (4575) S: | 0.022171 | 0.107793 | 0.018776 |
| (PLA) | Roseburia | | Dorea | | | |
| | faecis | | formicigenerans | | | |
| beta- | (2301) S: | 0.022147 | (6179) G: | 0.020411 | 0.107633 | 0.018953 |
| citrylglutamate | Alistipes | | Clostridium | | | |
| | finegoldii | | | | | |
| X - 14314 | (17241) S: | 0.014687 | (15154) F: | 0.013906 | 0.107403 | 0.019211 |
| | Bifidobacterium | | Clostridiales | | | |
| | catenulatum | | unclassified | | | |
| creatine | (5803) S: | −0.01668 | (4953) S: | −0.01582 | 0.107388 | 0.019228 |
| | Dialister sp | | Roseburia | | | |
| | CAG 357 | | sp CAG | | | |
| | | | 182 | | | |
| arabitol/xylitol | (1934) S: | 0.031652 | (4828) S: | 0.029975 | 0.106438 | 0.020327 |
| | Parabacteroides | | Blautia sp | | | |
| | distasonis | | | | | |
| uridine | (4547) S: | −0.03528 | (1790) S: | 0.026747 | 0.106231 | 0.020575 |
| | Anaerostipes | | Odoribacter | | | |
| | hadrus | | splanchnicus | | | |
| ectoine | (5062) G: | 0.010402 | (15326) G: | 0.008021 | 0.106182 | 0.020634 |
| | Firmicutes | | Faecalibacterium | | | |
| | unclassified | | | | | |
| X - 17653 | (4767) U: | −0.01285 | (4581) S: | 0.01176 | 0.10604 | 0.020805 |
| | Unknown | | Dorea | | | |
| | | | longicatena | | | |
| catechol | (6747) S: | −0.0224 | (15081) F: | 0.022163 | 0.105927 | 0.020941 |
| glucuronide | Clostridium | | Clostridiales | | | |
| | spiroforme | | unclassified | | | |
| X - 18887 | (15299) G: | 0.02692 | (15316) S: | 0.021941 | 0.104673 | 0.022516 |
| | Gemmiger | | Faecalibacterium | | | |
| | | | prausnitzii | | | |
| eicosapentaenoylcholine | (6141) F: | 0.048926 | (4303) S: | 0.030017 | 0.104352 | 0.022934 |
| | Peptostrep- | | Clostridium | | | |
| | tococcaceae | | sp CAG | | | |
| | | | 217 | | | |
| oleate/vaccenate | (5111) S: | 0.013939 | (14993) S: | 0.012976 | 0.104015 | 0.023382 |
| (18:1) | Clostridium sp | | Butyricicoccus | | | |
| | CAG 127 | | sp | | | |
| N- | (4829) S: | −0.01515 | (5087) S: | 0.012105 | 0.103957 | 0.02346 |
| acetylneuraminate | Blautia sp | | Eubacterium | | | |
| | | | sp CAG | | | |
| | | | 86 | | | |
| X - 16576 | (13981) U: | 0.013445 | (15460) F: | 0.008498 | 0.10394 | 0.023483 |
| | Unknown | | Desulfovibrionaceae | | | |
| X - 21839 | (15265) S: | 0.023227 | (4826) S: | −0.0224 | 0.103797 | 0.023675 |
| | Firmicutes | | Blautia sp | | | |
| | bacterium | | | | | |
| | CAG 103 | | | | | |
| 1-palmitoyl-2- | (4938) S: | −0.02856 | (14991) F: | 0.023705 | 0.103762 | 0.023723 |
| gamma- | Roseburia sp | | Clostridiaceae | | | |
| linolenoyl-GPC | | | | | | |
| (16:0/18:3n6)* | | | | | | |
| 2- | (17278) S: | −0.00852 | (14992) G: | 0.008154 | 0.103757 | 0.02373 |
| aminoheptanoate | Bifidobacterium | | Butyricicoccus | | | |
| | animalis | | | | | |
| palmitoyl | (4705) S: | −0.01695 | (4540) S: | 0.016293 | 0.103605 | 0.023936 |
| sphingomyelin | Clostridium sp | | Anaerostipes | | | |
| (d18:1/16:0) | CAG 43 | | hadrus | | | |
| nervonoylcarnitine | (15216) F: | −0.04038 | (4868) S: | −0.03403 | 0.103484 | 0.024102 |
| (C24:1)* | Clostridiales | | Blautia sp | | | |
| | unclassified | | | | | |
| X - 24812 | (6750) S: | 0.039472 | (4608) S: | 0.033314 | 0.103171 | 0.024536 |
| | Clostridium sp | | Ruminococcus | | | |
| | | | torques | | | |
| piperine | (6369) S: | −0.02014 | (15073) G: | −0.01911 | 0.102993 | 0.024785 |
| | Clostridium sp | | Oscillibacter | | | |
| | CAG 389 | | | | | |
| chiro-inositol | (14334) S: | −0.01845 | (4706) F: | 0.012575 | 0.101795 | 0.026521 |
| | Cryptobacterium | | Clostridiaceae | | | |
| | sp CAG | | | | | |
| | 338 | | | | | |
| X - 23974 | (713) G: | −0.03698 | (15154) F: | 0.030957 | 0.101544 | 0.026898 |
| | Methanobrevibacter | | Clostridiales | | | |
| | | | unclassified | | | |
| 3- | (17244) S: | −0.01941 | (4537) S: | −0.01776 | 0.101505 | 0.026957 |
| methoxycatechol | Bifidobacterium | | Eubacterium hallii | | | |
| sulfate (1) | adolescentis | | | | | |
| N-trimethyl 5- | (15271) S: | 0.026124 | (6141) F: | −0.02273 | 0.101107 | 0.027565 |
| aminovalerate | Ruthenibacterium | | Peptostrep- | | | |
| | lactatiformans | | tococcaceae | | | |
| glycochenodeoxycholate | (15291) F: | −0.02158 | (4914) S: | −0.02153 | 0.100996 | 0.027737 |
| glucuronide (1) | Ruminococcaceae | | Clostridium sp | | | |
| sphingomyelin | (4704) F: | −0.02206 | (15317) S: | −0.02017 | 0.100893 | 0.027897 |
| (d18:1/20:1, | Clostridiaceae | | Faecalibacterium | | | |
| d18:2/20:0)* | | | sp | | | |
| | | | CAG 82 | | | |
| X - 11470 | (14937) U: | −0.02188 | (4581) S: | 0.016799 | 0.100798 | 0.028046 |
| | Unknown | | Dorea | | | |
| | | | longicatena | | | |
| X - 21353 | (15256) F: | 0.0146 | (4936) S: | 0.014299 | 0.100654 | 0.028272 |
| | Clostridiales | | Roseburia | | | |
| | unclassified | | hominis | | | |
| X - 12472 | (14999) U: | 0.026062 | (14823) F: | 0.023754 | 0.100186 | 0.029017 |
| | Unknown | | Eggerthellaceae | | | |
| X - 12456 | (4705) S: | 0.015677 | (6962) S: | 0.015445 | 0.099379 | 0.030344 |
| | Clostridium sp | | Megamonas | | | |
| | CAG 43 | | funiformis | | | |
| X - 13866 | (15452) S: | 0.019139 | (6174) S: | 0.016247 | 0.098839 | 0.031261 |
| | Bilophila sp 4 | | Clostridium | | | |
| | 1 30 | | sp CAG | | | |
| | | | 265 | | | |
| vanillactate | (4831) F: | 0.051625 | (4824) G: | 0.041918 | 0.098677 | 0.031539 |
| | Lachnospiraceae | | Blautia | | | |
| X - 16580 | (1934) S: | 0.024707 | (15124) F: | 0.024448 | 0.098546 | 0.031768 |
| | Parabacteroides | | Clostridiales | | | |
| | distasonis | | unclassified | | | |
| X - 24329 | (2303) S: | −0.02538 | (1836) S: | −0.02536 | 0.09853 | 0.031795 |
| | Alistipes | | Bacteroides | | | |
| | finegoldii | | uniformis | | | |
| androsterone | (8076) S: | −0.02058 | (4839) G: | 0.018667 | 0.098384 | 0.03205 |
| glucuronide | Streptococcus | | Blautia | | | |
| | parasanguinis | | | | | |
| hydroxyasparagine** | (17248) S: | −0.00929 | (6141) F: | −0.00872 | 0.098378 | 0.032062 |
| | Bifidobacterium | | Peptostrep- | | | |
| | longum | | tococcaceae | | | |
| X - 23680 | (15374) F: | −0.02656 | (17241) S: | 0.025397 | 0.09835 | 0.032111 |
| | Ruminococcaceae | | Bifidobacterium | | | |
| | | | catenulatum | | | |
| 1- | (1903) S: | 0.028354 | (5075) S: | 0.028138 | 0.098016 | 0.032702 |
| oleoylglycerol | Bacteroides | | Lachnospira | | | |
| (18:1) | plebeius CAG | | pectinoschiza | | | |
| | 211 | | | | | |
| 1-(1-enyl- | (4780) G: | 0.014535 | (1790) S: | 0.011529 | 0.09774 | 0.033198 |
| palmitoyl)-2- | Clostridium | | Odoribacter | | | |
| palmitoleoyl- | | | splanchnicus | | | |
| GPC | | | | | | |
| (P-16:0/16:1)* | | | | | | |
| heneicosapentaenoate | (14400) G: | −0.0271 | (6141) F: | 0.01887 | 0.096856 | 0.03483 |
| (21:5n3) | Collinsella | | Peptostrep- | | | |
| | | | tococcaceae | | | |
| N-palmitoyl- | (15342) S: | −0.02804 | (1957) S: | 0.027896 | 0.096756 | 0.035019 |
| heptadecasphingosine | Faecalibacterium | | Bacteroides | | | |
| (d17:1/16:0)* | prausnitzii | | sp CAG | | | |
| | | | 144 | | | |
| beta-alanine | (6148) F: | 0.020157 | (4925) S: | 0.020075 | 0.096348 | 0.035799 |
| | Peptostrep- | | Roseburia | | | |
| | tococcaceae | | faecis | | | |
| X - 21474 | (15318) S: | −0.04014 | (4659) S: | 0.039439 | 0.096222 | 0.036041 |
| | Faecalibacterium | | Clostridium | | | |
| | prausnitzii | | sp CAG | | | |
| | | | 122 | | | |
| 2- | (15350) U: | 0.051887 | (15124) F: | 0.035101 | 0.095939 | 0.036596 |
| docosahexaenoylglycerol | Unknown | | Clostridiales | | | |
| (22:6)* | | | unclassified | | | |
| margarate | (14974) U: | 0.013311 | (4940) S: | 0.012762 | 0.095892 | 0.036687 |
| (17:0) | Unknown | | Roseburia | | | |
| | | | inulinivorans | | | |
| 1-ribosyl- | (15342) S: | 0.023475 | (4957) F: | 0.022928 | 0.095809 | 0.03685 |
| imidazoleacetate* | Faecalibacterium | | Eubacteriaceae | | | |
| | prausnitzii | | | | | |
| X - 21295 | (4669) G: | −0.02087 | (14861) U: | 0.020517 | 0.095321 | 0.037827 |
| | Coprococcus | | Unknown | | | |
| cysteinylglycine | (14020) U: | −0.02178 | (15286) F: | −0.02115 | 0.09521 | 0.038051 |
| disulfide* | Unknown | | Ruminococcaceae | | | |
| tryptophan | (15054) F: | −0.01383 | (8002) S: | 0.01105 | 0.094892 | 0.038701 |
| | Clostridiales | | Streptococcus | | | |
| | unclassified | | thermophilus | | | |
| 1-palmitoyl-2- | (15229) F: | −0.01867 | (4121) U: | −0.01846 | 0.094768 | 0.038959 |
| docosahexaenoyl- | Clostridiales | | Unknown | | | |
| GPE | unclassified | | | | | |
| (16:0/22:6)* | | | | | | |
| S- | (15332) S: | 0.043284 | (4882) S: | 0.042659 | 0.094733 | 0.039031 |
| adenosylhomocysteine | Faecalibacterium | | Roseburia | | | |
| (SAH) | prausnitzii | | sp CAG | | | |
| | | | 100 | | | |
| X - 12206 | (4959) S: | −0.02691 | (4546) S: | 0.019168 | 0.094575 | 0.03936 |
| | Eubacterium | | Eubacterium | | | |
| | ramulus | | sp | | | |
| X - 18345 | (4394) U: | 0.012098 | (9701) S: | 0.010524 | 0.094256 | 0.040032 |
| | Unknown | | Haemophilus | | | |
| | | | sp | | | |
| | | | HMSC061E01 | | | |
| tauro-beta- | (4831) F: | −0.03274 | (4130) U: | −0.03214 | 0.094251 | 0.040043 |
| muricholate | Lachnospiraceae | | Unknown | | | |
| phenylpyruvate | (14932) U: | −0.0089 | (9226) S: | −0.00696 | 0.09316 | 0.042412 |
| | Unknown | | Akkermansia | | | |
| | | | muciniphila | | | |
| oleoyl | (1814) S: | 0.017731 | (13982) U: | 0.01504 | 0.092823 | 0.043169 |
| ethanolamide | Bacteroides | | Unknown | | | |
| | vulgatus | | | | | |
| 2,3- | (14993) S: | 0.01931 | (14416) G: | −0.01704 | 0.092506 | 0.04389 |
| dihydroxyisovalerate | Butyricicoccus | | Collinsella | | | |
| | sp | | | | | |
| X - 16964 | (4537) S: | −0.0594 | (4914) S: | −0.05497 | 0.092354 | 0.044241 |
| | Eubacterium | | Clostridium sp | | | |
| | hallii | | | | | |
| X - 12544 | (4564) S: | 0.005718 | (14252) U: | 0.005618 | 0.092332 | 0.04429 |
| | Ruminococcus | | Unknown | | | |
| | torques | | | | | |
| arachidate | (15346) G: | 0.019826 | (15154) F: | 0.018477 | 0.092187 | 0.044628 |
| (20:0) | Faecalibacterium | | Clostridiales | | | |
| | | | unclassified | | | |
| X - 17655 | (6472) F: | 0.01049 | (4782) U: | 0.007802 | 0.091978 | 0.045114 |
| | Clostridiaceae | | Unknown | | | |
| 5alpha- | (8002) S: | −0.02357 | (15091) G: | 0.022201 | 0.091942 | 0.045199 |
| pregnan- | Streptococcus | | Oscillibacter | | | |
| 3beta,20alpha- | thermophilus | | | | | |
| diol disulfate | | | | | | |
| X - 15486 | (4644) S: | −0.01694 | (4826) S: | 0.016926 | 0.091313 | 0.046698 |
| | Clostridium sp | | Blautia sp | | | |
| | CAG 62 | | | | | |
| 3,7- | (1832) S: | 0.018158 | (4537) S: | −0.01529 | 0.091072 | 0.047282 |
| dimethylurate | Bacteroides | | Eubacterium | | | |
| | clarus | | hallii | | | |

According to a particular embodiment, the metabolite which is predicted is set forth in Table 4.

TABLE 4

| | Top | Directional | Top | Directional | Top | Directional |
|---|---|---|---|---|---|---|
| | predictor | SHAP value | predictor | SHAP value | predictor | SHAP value |
| BIOCHEMICAL | #1 | #1 | #2 | #2 | #3 | #3 |
| 1-methylxanthine | Coffee Freq | 0.521955 | SF_Coffee_wt | 0.453195 | SF_Cappuccino_wt | 0.078874 |
| 3-carboxy-4- | Fish Cooked, | 0.382587 | Canned Tuna | 0.149771 | Fish (not | 0.107626 |
| methyl-5- | Baked or | | or Tuna Salad | | Tuna) Pickled, | |
| propyl-2- | Grilled Freq | | Freq | | Dried, Smoked, | |
| furanpropanoate | | | | | Canned Freq | |
| (CMPF) | | | | | | |
| hydroxy-CMPF* | Fish Cooked, | 0.373131 | Canned Tuna | 0.165863 | Fish (not | 0.139629 |
| | Baked or | | or Tuna Salad | | Tuna) Pickled, | |
| | Grilled Freq | | Freq | | Dried, Smoked, | |
| | | | | | Canned Freq | |
| quinate | SF_Coffee_wt | 0.366101 | Coffee Freq | 0.301928 | SF_Cappuccino_wt | 0.073144 |
| X - 21442 | SF_Coffee_wt | 0.507797 | Coffee Freq | 0.391595 | SF_Cappuccino_wt | 0.142793 |
| 1-methylurate | SF_Coffee_wt | 0.449174 | Coffee Freq | 0.385187 | SF_Cappuccino_wt | 0.101257 |
| 1,3-dimethylurate | Coffee Freq | 0.508676 | SF_Coffee_wt | 0.439991 | SF_Cappuccino_wt | 0.125518 |
| 1,3,7-trimethylurate | Coffee Freq | 0.52283 | SF_Coffee_wt | 0.425254 | SF_Cappuccino_wt | 0.086449 |
| X - 24811 | SF_Coffee_wt | 0.528661 | Coffee Freq | 0.442288 | SF_Cappuccino_wt | 0.094478 |
| theophylline | Coffee Freq | 0.428521 | SF_Coffee_wt | 0.399509 | SF_Cappuccino_wt | 0.088292 |
| 5-acetylamino- | SF_Coffee_wt | 0.469622 | Coffee Freq | 0.403792 | 3% Milk Freq | 0.061279 |
| 6-amino-3- | | | | | | |
| methyluracil | | | | | | |
| 1,7-dimethylurate | Coffee Freq | 0.472547 | SF_Coffee_wt | 0.460378 | SF_Cappuccino_wt | 0.06662 |
| caffeine | Coffee Freq | 0.419314 | SF_Coffee_wt | 0.350417 | SF_Wine_wt | 0.0539 |
| paraxanthine | Coffee Freq | 0.541851 | SF_Coffee_wt | 0.467303 | SF_Cappuccino_wt | 0.097286 |
| X - 23655 | SF_Coffee_wt | 0.435476 | Coffee Freq | 0.303309 | SF_Cappuccino_wt | 0.083376 |
| X - 13835 | Pastrami or | 0.188407 | Beef, Veal, | 0.187234 | SF_WhiteWheat_g_wt | 0.113279 |
| | Smoked Turkey | | Lamb, Pork, | | | |
| | Breast Freq | | Steak, Golash | | | |
| | | | Freq | | | |
| saccharin | Artificial | 0.312888 | SF_Sugar | 0.111067 | Oil as an | −0.0301 |
| | Sweeteners | | substitute_wt | | addition for | |
| | Freq | | | | Salads or | |
| | | | | | Stews Freq | |
| 3-methyl catechol | SF_Coffee_wt | 0.276915 | Coffee Freq | 0.268574 | SF_Wine_wt | 0.051132 |
| sulfate (1) | | | | | | |
| 3-hydroxypyridine | SF_Coffee_wt | 0.295696 | Coffee Freq | 0.21339 | Ice Cream or | −0.04483 |
| sulfate | | | | | Popsicle which | |
| | | | | | contains | |
| | | | | | Dairy Freq | |
| X - 23652 | Beef, Veal, | 0.143448 | Pastrami or | 0.115295 | SF_WhiteWheat_g_wt | 0.076963 |
| | Lamb, Pork, | | Smoked Turkey | | | |
| | Steak, Golash | | Breast Freq | | | |
| | Freq | | | | | |
| trigonelline (N′- | SF_Coffee_wt | 0.263854 | Coffee Freq | 0.215773 | SF_Cappuccino_wt | 0.060521 |
| methylnicotinate) | | | | | | |
| X - 11315 | SF_Almonds_wt | 0.206196 | Nuts, | 0.138389 | SF_Milk_wt | −0.12129 |
| | | | almonds, | | | |
| | | | pistachios | | | |
| | | | Freq | | | |
| 1-methyl-5- | Beef, Veal, | 0.123294 | Pastrami or | 0.095776 | SF_WhiteWheat_g_wt | 0.091505 |
| imidazoleacetate | Lamb, Pork, | | Smoked Turkey | | | |
| | Steak, Golash | | Breast Freq | | | |
| | Freq | | | | | |
| 1-(1-enyl-palmitoyl)- | Chicken or | 0.131388 | Turkey | 0.104297 | Beef, Veal, | 0.103265 |
| 2-arachidonoyl-GPE | Turkey | | Meatballs, | | Lamb, Pork, | |
| (P-16:0/20:4)* | Without Skin | | Beef, Chicken | | Steak, Golash | |
| | Freq | | Freq | | Freq | |
| X - 11858 | SF_Tahini_wt | 0.326823 | Tahini Salad | 0.171167 | SF_Hummus | 0.068006 |
| | | | Freq | | Salad_wt | |
| 1-(1-enyl-stearoyl)- | Egg, Hard | 0.1218 | Beef, Veal, | 0.108988 | Turkey | 0.103305 |
| 2-arachidonoyl-GPE | Boiled or Soft | | Lamb, Pork, | | Meatballs, | |
| (P-18:0/20:4)* | Freq | | Steak, Golash | | Beef, Chicken | |
| | | | Freq | | Freq | |
| X - 21339 | Fries Freq | 0.212512 | Falafel in Pita | 0.093116 | SF_Apple_wt | −0.08103 |
| | | | Bread Freq | | | |
| 3-methylhistidine | Beef, Veal, | 0.106555 | Pastrami or | 0.102308 | Chicken or | 0.09838 |
| | Lamb, Pork, | | Smoked Turkey | | Turkey | |
| | Steak, Golash | | Breast Freq | | Without Skin | |
| | Freq | | | | Freq | |
| X - 23649 | SF_Coffee_wt | 0.445723 | Coffee Freq | 0.321779 | Mixed | 0.090503 |
| | | | | | Chicken or | |
| | | | | | Turkey Dishes | |
| | | | | | Freq | |
| 4-ethylcatechol | SF_Coffee_wt | 0.331396 | Coffee Freq | 0.238634 | SF_WhiteWheat_g_wt | −0.03928 |
| sulfate | | | | | | |
| X - 11880 | Fries Freq | 0.206379 | Falafel in Pita | 0.087521 | SF_Natural | −0.07837 |
| | | | Bread Freq | | Yogurt_wt | |
| X - 11308 | Fries Freq | 0.138526 | Alcoholic | 0.106923 | SF_Hummus | 0.080083 |
| | | | Drinks Freq | | Salad_wt | |
| 2,3-dihydroxypyridine | Coffee Freq | 0.453341 | SF_Coffee_wt | 0.418166 | SF_Bread_wt | −0.06984 |
| beta-cryptoxanthin | Mandarin or | 0.187788 | Red Pepper | 0.147568 | Persimmon | 0.11856 |
| | Clementine | | Freq | | Freq | |
| | Freq | | | | | |
| X - 13844 | SF_Coffee_wt | 0.3579 | Coffee Freq | 0.274482 | Regular Sodas | −0.11742 |
| | | | | | with Sugar | |
| | | | | | Freq | |
| X - 11372 | Fries Freq | 0.159375 | Salty Snacks | 0.10569 | Alcoholic | 0.073839 |
| | | | Freq | | Drinks Freq | |
| 1-palmitoyl-2- | Fish Cooked, | 0.252315 | Canned Tuna | 0.100372 | Fish (not | 0.064741 |
| docosahexaenoyl-GPC | Baked or | | or Tuna Salad | | Tuna) Pickled, | |
| (16:0/22:6) | Grilled Freq | | Freq | | Dried, Smoked, | |
| | | | | | Canned Freq | |
| X - 24949 | SF_Tahini_wt | 0.19913 | Tahini Salad | 0.151 | SF_Olive | 0.061803 |
| | | | Freq | | oil_wt | |
| X - 18914 | 3% Milk Freq | 0.125681 | Cooked | −0.10398 | SF_Milk_wt | 0.097464 |
| | | | Legumes Freq | | | |
| X - 21661 | SF_Tahini_wt | 0.327191 | Tahini Salad | 0.141354 | Hummus | 0.092048 |
| | | | Freq | | Salad Freq | |
| sphingomyelin | >=16% Yellow | 0.094716 | 3% Milk Freq | 0.081685 | Cooked | −0.07917 |
| (d17:1/16:0, | Cheese Freq | | | | Legumes Freq | |
| d18:1/15:0, | | | | | | |
| d16:1/17:0)* | | | | | | |
| X - 21752 | Cooked | 0.27247 | Granola or | 0.189121 | SF_Granola_wt | 0.100119 |
| | Cereal such as | | Bernflaks | | | |
| | Oatmeal | | Freq | | | |
| | Porridge Freq | | | | | |
| X - 12816 | SF_Coffee_wt | 0.50891 | Coffee Freq | 0.271773 | SF_Cappuccino_wt | 0.155256 |
| 5alpha-androstan- | Beer Freq | 0.165139 | SF_Beer_wt | 0.130197 | SF_WhiteWheat_g_wt | 0.102007 |
| 3alpha,17beta-diol | | | | | | |
| monosulfate (2) | | | | | | |
| stachydrine | SF_Orange_wt | 0.156365 | Mandarin or | 0.093164 | SF_Vegetable | 0.060348 |
| | | | Clementine | | Salad_wt | |
| | | | Freq | | | |
| X - 23639 | SF_Coffee_wt | 0.129872 | Coffee Freq | 0.080549 | SF_Omelette_wt | −0.06285 |
| sphingomyelin | >=16% Yellow | 0.095505 | SF_Milk_wt | 0.077198 | Beef or | 0.072712 |
| (d18:1/17:0, | Cheese Freq | | | | Chicken Soup | |
| d17:1/18:0, | | | | | Freq | |
| d19:1/16:0) | | | | | | |
| X - 11381 | 3% Milk Freq | 0.185143 | SF_Coffee_wt | 0.128715 | SF_Milk_wt | 0.069382 |
| X - 24637 | SF_Soymilk_wt | 0.20839 | SF_Tofu_wt | 0.051064 | Beef, Veal, | −0.02708 |
| | | | | | Lamb, Pork, | |
| | | | | | Steak, Golash | |
| | | | | | Freq | |
| X - 17185 | SF_Coffee_wt | 0.39039 | Coffee Freq | 0.19712 | SF_Salmon_wt | −0.07847 |
| 5-acetylamino-6- | SF_Coffee_wt | 0.33866 | Coffee Freq | 0.238393 | SF_Tomatoes_wt | −0.04626 |
| formylamino- | | | | | | |
| 3-methyluracil | | | | | | |
| X - 17145 | SF_Apple_wt | 0.194117 | SF_Orange_wt | 0.112769 | Apple Freq | 0.092335 |
| X - 11847 | SF_Tahini_wt | 0.295026 | Tahini Salad | 0.136155 | Hummus | 0.082698 |
| | | | Freq | | Salad Freq | |
| 1,5-anhydroglucitol | Regular Sodas | 0.138944 | SF_WhiteWheat_g_wt | 0.112928 | Ordinary | 0.082267 |
| (1,5-AG) | with Sugar | | | | Bread or | |
| | Freq | | | | Challah Freq | |
| X - 18249 | SF_Olive | −0.11992 | Cooked | −0.10672 | 3% Milk Freq | 0.101731 |
| | oil_wt | | Legumes Freq | | | |
| citraconate/ | Coffee Freq | 0.232123 | SF_Coffee_wt | 0.199446 | SF_Rice | 0.047645 |
| glutaconate | | | | | crackers_wt | |
| X - 12329 | SF_Coffee_wt | 0.319652 | Coffee Freq | 0.227085 | SF_Bread_wt | −0.07622 |
| sphingomyelin | SF_Milk_wt | 0.102712 | Cooked | −0.08776 | Hummus | −0.07391 |
| (d18:1/19:0, | | | Legumes Freq | | Salad Freq | |
| d19:1/18:0)* | | | | | | |
| X - 14939 | SF_Tahini_wt | 0.115425 | SF_Hummus | 0.08575 | Falafel in Pita | 0.062322 |
| | | | Salad_wt | | Bread Freq | |
| acesulfame | Diet Soda | 0.247531 | Artificial | 0.154245 | SF_Sugar Free | 0.084721 |
| | Freq | | Sweeteners | | Gum_wt | |
| | | | Freq | | | |
| 1-stearoyl-2- | Fish Cooked, | 0.224374 | Canned Tuna | 0.098494 | Fish (not | 0.065521 |
| docosahexaenoyl-GPC | Baked or | | or Tuna Salad | | Tuna) Pickled, | |
| (18:0/22:6) | Grilled Freq | | Freq | | Dried, Smoked, | |
| | | | | | Canned Freq | |
| 5alpha-androstan- | Beer Freq | 0.193374 | SF_WhiteWheat_g_wt | 0.128321 | SF_Beer_wt | 0.118443 |
| 3alpha,17beta- | | | | | | |
| diol disulfate | | | | | | |
| tryptophan | Cooked | 0.194321 | SF_Tahini_wt | 0.090885 | Beef or | −0.05859 |
| betaine | Legumes Freq | | | | Chicken Soup | |
| | | | | | Freq | |
| gamma- | Cooked | −0.08704 | SF_Parsley_wt | −0.07333 | SF_WhiteWheat_g_wt | 0.065157 |
| glutamylvaline | Legumes Freq | | | | | |
| daidzein | SF_Soymilk_wt | 0.150796 | SF_Tofu_wt | 0.03136 | Cooked | 0.020687 |
| sulfate (2) | | | | | Legumes Freq | |
| sphingomyelin | 3% Milk Freq | 0.109429 | 3-5% Natural | 0.108154 | >=16% Yellow | 0.095027 |
| (d18:1/25:0, | | | Yogurt Freq | | Cheese Freq | |
| d19:0/24:1, | | | | | | |
| d20:1/23:0, | | | | | | |
| d19:1/24:0)* | | | | | | |
| sphingomyelin | Cooked | −0.09994 | SF_Milk_wt | 0.074219 | 0.5-3% White | 0.063754 |
| (d18:1/14:0, | Legumes Freq | | | | Cheese, | |
| d16:1/16:0)* | | | | | Cottage Freq | |
| X - 24475 | SF_Almonds_wt | 0.180157 | Nuts, | 0.135553 | Apple Freq | 0.101342 |
| | | | almonds, | | | |
| | | | pistachios | | | |
| | | | Freq | | | |
| methyl | SF_Butter_wt | −0.09535 | Orange or | 0.093033 | SF_Banana_wt | 0.086389 |
| glucopyranoside | | | Grapefruit | | | |
| (alpha + beta) | | | Freq | | | |
| X - 11795 | SF_WhiteWheat_g_wt | 0.127225 | Pasta or | 0.125764 | SF_WholeWheat_g_wt | 0.108236 |
| | | | Flakes Freq | | | |
| docosahexaenoate | Fish Cooked, | 0.183304 | Fish (not | 0.098234 | Canned Tuna | 0.085462 |
| (DHA; 22:6n3) | Baked or | | Tuna) Pickled, | | or Tuna Salad | |
| | Grilled Freq | | Dried, Smoked, | | Freq | |
| | | | Canned Freq | | | |
| X - 11849 | SF_Tahini_wt | 0.26333 | Tahini Salad | 0.122309 | Hummus | 0.089613 |
| | | | Freq | | Salad Freq | |
| X - 18922 | SF_Tahini_wt | 0.1229 | SF_Olive | 0.088716 | Peach, | −0.06299 |
| | | | oil_wt | | Nectarine, | |
| | | | | | Plum Freq | |
| S-methylcysteine | Brussels | 0.120063 | SF_Cooked | 0.05419 | SF_Kohlrabi_wt | 0.050796 |
| sulfoxide | Sprouts, | | cauliflower_wt | | | |
| | Green or Red | | | | | |
| | Cabbage Freq | | | | | |
| perfluorooctane- | Fish Cooked, | 0.122487 | Fish (not | 0.093453 | Simple | −0.0496 |
| sulfonic acid | Baked or | | Tuna) Pickled, | | Cookies or | |
| (PFOS) | Grilled Freq | | Dried, Smoked, | | Biscuits Freq | |
| | | | Canned Freq | | | |
| 3-hydroxystachydrine* | SF_Orange_wt | 0.173077 | Mandarin or | 0.134784 | SF_Plum_wt | −0.07661 |
| | | | Clementine | | | |
| | | | Freq | | | |
| sphingomyelin | SF_Milk_wt | 0.111512 | Hummus | −0.0925 | Beer Freq | −0.06966 |
| (d18:2/23:1)* | | | Salad Freq | | | |
| maleate | Coffee Freq | 0.211948 | SF_Coffee_wt | 0.149481 | SF_Rice | 0.05272 |
| | | | | | crackers_wt | |
| eicosenedioate | SF_WhiteWheat_g_wt | 0.102584 | SF_Apple_wt | −0.08961 | Fries Freq | 0.0863 |
| (C20:1-DC)* | | | | | | |
| homostachydrine* | SF_Coffee_wt | 0.226189 | SF_Wholemeal | 0.083208 | SF_WholeWheat_g_wt | 0.080145 |
| | | | Bread_wt | | | |
| creatine | Turkey | 0.099401 | Chicken or | 0.090823 | Artificial | 0.056639 |
| | Meatballs, | | Turkey | | Sweeteners | |
| | Beef, Chicken | | Without Skin | | Freq | |
| | Freq | | Freq | | | |
| X - 17653 | Falafel in Pita | 0.121756 | Fries Freq | 0.07962 | SF_WhiteWheat_g_wt | 0.069756 |
| | Bread Freq | | | | | |
| catechol | SF_Coffee_wt | 0.26583 | Coffee Freq | 0.161844 | Herbal Tea | 0.070285 |
| sulfate | | | | | Freq | |
| X - 16935 | Fries Freq | 0.22235 | SF_Tahini_wt | −0.11794 | Small Burekas | 0.093917 |
| | | | | | Freq | |
| sphingomyelin | Beer Freq | −0.10449 | Hummus | −0.08009 | SF_Coffee_wt | 0.065897 |
| (d18:2/21:0, | | | Salad Freq | | | |
| d16:2/23:0)* | | | | | | |
| sphingomyelin | Cooked | −0.10394 | Beer Freq | −0.06772 | 0.5-3% White | 0.062997 |
| (d17:2/16:0, | Legumes Freq | | | | Cheese, | |
| d18:2/15:0)* | | | | | Cottage Freq | |
| S-methylcysteine | Brussels | 0.094394 | SF_Lentils_wt | 0.052955 | SF_Vegetable | 0.041488 |
| | Sprouts, | | | | Soup_wt | |
| | Green or Red | | | | | |
| | Cabbage Freq | | | | | |
| N-(2-furoyl)glycine | SF_Coffee_wt | 0.232673 | Coffee Freq | 0.140282 | SF_Wine_wt | 0.03347 |
| 2,6-dihydroxybenzoic | Cooked | 0.09138 | Couscous, | 0.060169 | Granola or | 0.057334 |
| acid | Cereal such as | | Burgul, | | Bernflaks | |
| | Oatmeal | | Mamaliga, | | Freq | |
| | Porridge Freq | | Groats Freq | | | |
| X - 12837 | Coffee Freq | 0.280958 | SF_Coffee_wt | 0.218618 | SF_Cappuccino_wt | 0.069369 |
| pyroglutamine* | Beer Freq | 0.107215 | Mayonnaise | −0.08141 | Falafel in Pita | 0.066716 |
| | | | Including | | Bread Freq | |
| | | | Light Freq | | | |
| N-delta- | Red Pepper | 0.12734 | Cooked | 0.10344 | SF_Apple_wt | 0.087097 |
| acetylornithine | Freq | | Legumes Freq | | | |
| X - 21736 | SF_Butter_wt | 0.126458 | SF_Carrots_wt | −0.08728 | SF_Tomatoes_wt | −0.07812 |
| tridecenedioate | Tahini Salad | −0.16699 | SF_Tahini_wt | −0.12043 | SF_Soymilk_wt | −0.10931 |
| (C13:1-DC)* | Freq | | | | | |
| heneicosa- | Fish Cooked, | 0.144003 | Fish (not | 0.122323 | SF_Rice_wt | −0.07452 |
| pentaenoate | Baked or | | Tuna) Pickled, | | | |
| (21:5n3) | Grilled Freq | | Dried, Smoked, | | | |
| | | | Canned Freq | | | |
| 2-aminobutyrate | Simple | −0.06513 | Chicken or | 0.06163 | Beef or | 0.051808 |
| | Cookies or | | Turkey With | | Chicken Soup | |
| | Biscuits Freq | | Skin Freq | | Freq | |
| X - 11378 | Alcoholic | 0.103051 | Beer Freq | 0.087346 | SF_WhiteWheat_g_wt | 0.075742 |
| | Drinks Freq | | | | | |
| 2-hydroxylaurate | Fries Freq | 0.097427 | Alcoholic | 0.089771 | SF_Apple_wt | −0.07034 |
| | | | Drinks Freq | | | |
| 17-methylstearate | Butter Freq | 0.101184 | Simple | −0.08125 | Beef, Veal, | 0.057793 |
| | | | Cookies or | | Lamb, Pork, | |
| | | | Biscuits Freq | | Steak, Golash | |
| | | | | | Freq | |
| 15-methylpalmitate | Butter Freq | 0.079326 | SF_Butter_wt | 0.059867 | 3% Milk Freq | 0.057259 |
| sphingomyelin | Beer Freq | −0.09521 | Honey, Jam, | 0.086719 | Cooked | −0.07236 |
| (d18:2/14:0, | | | fruit syrup, | | Legumes Freq | |
| d18:1/14:l)* | | | Maple syrup | | | |
| | | | Freq | | | |
| hippurate | SF_Coffee_wt | 0.288796 | Coffee Freq | 0.049863 | Fried Fish | −0.04174 |
| | | | | | Freq | |
| X - 12730 | SF_Coffee_wt | 0.251511 | Coffee Freq | 0.177172 | SF_Bread_wt | −0.07314 |
| 1-(1-enyl-palmitoyl)- | Beef, Veal, | 0.143527 | Egg Recipes | 0.072606 | Turkey | 0.05528 |
| 2-arachidonoyl-GPC | Lamb, Pork, | | Freq | | Meatballs, | |
| (P-16:0/20:4)* | Steak, Golash | | | | Beef, Chicken | |
| | Freq | | | | Freq | |
| caffeic acid | SF_Coffee_wt | 0.179171 | Coffee Freq | 0.138653 | Regular Sodas | −0.04717 |
| sulfate | | | | | with Sugar | |
| | | | | | Freq | |
| 1-(1-enyl- | Beef or | 0.110716 | Egg, Hard | 0.085947 | Beef, Veal, | 0.065351 |
| stearoyl)-GPE | Chicken Soup | | Boiled or Soft | | Lamb, Pork, | |
| (P-18:0)* | Freq | | Freq | | Steak, Golash | |
| | | | | | Freq | |
| 3-methyl catechol | Coffee Freq | 0.227497 | SF_Coffee_wt | 0.223543 | SF_Wine_wt | 0.071815 |
| sulfate (2) | | | | | | |
| oxalate | Red Pepper | 0.171087 | SF_Butter_wt | −0.06363 | SF_Cucumber_wt | 0.057747 |
| (ethanedioate) | Freq | | | | | |
| eicosapentaenoate | Fish (not | 0.087437 | Fish Cooked, | 0.083989 | SF_Tahini_wt | −0.07324 |
| (EPA; 20:5n3) | Tuna) Pickled, | | Baked or | | | |
| | Dried, Smoked, | | Grilled Freq | | | |
| | Canned Freq | | | | | |
| X - 12738 | SF_Coffee_wt | 0.292468 | Coffee Freq | 0.264584 | SF_Wine_wt | 0.083477 |
| X - 21383 | SF_Hummus | 0.056309 | 5-9% Yellow | 0.054893 | 5-9% White | 0.051581 |
| | Salad_wt | | Cheese Freq | | Cheese, | |
| | | | | | Cottage Freq | |
| creatinine | Beer Freq | 0.091432 | SF_Beef_wt | 0.061359 | SF_WhiteWheat_g_wt | 0.058713 |
| gentisate | Cooked | 0.10068 | SF_Almonds_wt | 0.077111 | Wholemeal or | 0.063621 |
| | Legumes Freq | | | | Rye Bread | |
| | | | | | Freq | |
| X - 24951 | Fries Freq | 0.101159 | SF_WhiteWheat_g_wt | 0.07231 | Salty Snacks | 0.061788 |
| | | | | | Freq | |
| X - 17654 | SF_WhiteWheat_g_wt | 0.085536 | Falafel in Pita | 0.085105 | Fries Freq | 0.075289 |
| | | | Bread Freq | | | |
| tiglylcarnitine | Cooked | −0.07801 | SF_Omelette_wt | 0.075921 | Mango Freq | −0.06379 |
| (C5:1-DC) | Cereal such as | | | | | |
| | Oatmeal | | | | | |
| | Porridge Freq | | | | | |
| 2-aminoheptanoate | SF_Milk_wt | −0.08734 | SF_Tahini_wt | 0.061512 | Chicken or | −0.05331 |
| | | | | | Turkey | |
| | | | | | Without Skin | |
| | | | | | Freq | |
| phytanate | Butter Freq | 0.080496 | Beef, Veal, | 0.06686 | Corn Freq | −0.06446 |
| | | | Lamb, Pork, | | | |
| | | | Steak, Golash | | | |
| | | | Freq | | | |
| androsterone | Beer Freq | 0.133229 | SF_Coffee_wt | −0.06105 | Hummus | 0.046412 |
| glucuronide | | | | | Salad Freq | |
| 4-vinylguaiacol | SF_Coffee_wt | 0.272866 | Coffee Freq | 0.151908 | SF_Bread_wt | −0.10835 |
| sulfate | | | | | | |
| 1-docosahexaenoyl- | Fish Cooked, | 0.272759 | Fish (not | 0.079088 | Canned Tuna | 0.064288 |
| glycerol (22:6) | Baked or | | Tuna) Pickled, | | or Tuna Salad | |
| | Grilled Freq | | Dried, Smoked, | | Freq | |
| | | | Canned Freq | | | |
| 2-aminophenol | SF_WholeWheat _g_wt | 0.117974 | SF_Wholemeal | 0.109317 | Pasta or | 0.076604 |
| sulfate | | | Bread_wt | | Flakes Freq | |
| N2,N5-diacetylornithine | SF_Apple_wt | 0.105153 | Red Pepper | 0.095432 | Cooked | 0.077642 |
| | | | Freq | | Legumes Freq | |
| X - 17676 | SF_Coffee_wt | 0.200781 | Coffee Freq | 0.193388 | SF_Rice | 0.073746 |
| | | | | | crackers_wt | |
| carotene diol (2) | SF_WhiteWheat_g_wt | −0.05751 | Yeast Cakes | −0.05427 | SF_Chicken | −0.05335 |
| | | | and Cookies | | breast_wt | |
| | | | as Rogallach, | | | |
| | | | Croissant or | | | |
| | | | Donut Freq | | | |
| 4-ethylphenylsulfate | SF_Soymilk_wt | 0.146566 | SF_Tofu_wt | 0.059703 | Beef, Veal, | −0.05809 |
| | | | | | Lamb, Pork, | |
| | | | | | Steak, Golash | |
| | | | | | Freq | |
| 2-aminoadipate | Pastrami or | 0.074629 | SF_Sugar Free | −0.04829 | White or | −0.04431 |
| | Smoked Turkey | | Gum_wt | | Brown Sugar | |
| | Breast Freq | | | | Freq | |
| O-methylcatechol | SF_Coffee_wt | 0.252455 | Coffee Freq | 0.110436 | SF_Wine_wt | 0.060985 |
| sulfate | | | | | | |
| X - 24655 | SF_Soymilk_wt | 0.164445 | SF_Tofu_wt | 0.024294 | SF_Rice_wt | −0.02072 |
| ceramide | Artificial | 0.12836 | Cooked | −0.12107 | Coffee Freq | 0.090017 |
| (d18:1/14:0, | Sweeteners | | Legumes Freq | | | |
| d16:1/16:0)* | Freq | | | | | |
| X - 17325 | SF_Coffee_wt | 0.347227 | Coffee Freq | 0.068552 | Peach, | 0.044107 |
| | | | | | Nectarine, | |
| | | | | | Plum Freq | |
| N1-Methyl-2-pyridone- | Pastrami or | 0.090985 | 0-1.5% | 0.061739 | Roll or | −0.05568 |
| 5-carboxamide | Smoked Turkey | | Natural | | Bageles Freq | |
| | Breast Freq | | Yogurt Freq | | | |
| urate | SF_WhiteWheat_g_wt | 0.11539 | Chicken or | 0.058241 | Beer Freq | 0.057116 |
| | | | Turkey With | | | |
| | | | Skin Freq | | | |
| carotene diol (3) | Red Pepper | 0.245336 | SF_Orange_wt | 0.035475 | Wholemeal or | −0.03217 |
| | Freq | | | | Rye Bread | |
| | | | | | Freq | |
| 1-methylhistidine | Beef, Veal, | 0.096089 | Chicken or | 0.056173 | SF_WhiteWheat_g_wt | 0.054076 |
| | Lamb, Pork, | | Turkey With | | | |
| | Steak, Golash | | Skin Freq | | | |
| | Freq | | | | | |
| 3-acetylphenol | SF_Coffee_wt | 0.273419 | Coffee Freq | 0.212696 | SF_Salmon_wt | −0.03895 |
| sulfate | | | | | | |
| theobromine | Milk or Dark | 0.17203 | Coffee Freq | 0.127556 | SF_Coffee_wt | 0.084332 |
| | Chocolate | | | | | |
| | Freq | | | | | |
| N-methylproline | SF_Orange_wt | 0.165026 | Mandarin or | 0.082633 | Orange or | 0.055373 |
| | | | Clementine | | Grapefruit | |
| | | | Freq | | Freq | |
| dihydrocaffeate | SF_Coffee_wt | 0.27251 | Coffee Freq | 0.133393 | Pita Freq | −0.06401 |
| sulfate (2) | | | | | | |
| threonate | Red Pepper | 0.13263 | SF_WhiteWheat_g_wt | −0.06314 | SF_Apple_wt | 0.059448 |
| | Freq | | | | | |
| X - 12221 | SF_Coffee_wt | 0.29291 | SF_Tahini_wt | −0.06887 | SF_Peas_wt | −0.06465 |
| myristoyl | Butter Freq | 0.058086 | 3-5% Natural | 0.050942 | Coffee Freq | 0.050482 |
| dihydrosphingo- | | | Yogurt Freq | | | |
| myelin | | | | | | |
| (d18:0/14:0)* | | | | | | |
| X - 17367 | SF_Coffee_wt | 0.335257 | Pasta or | −0.04922 | Peach, | 0.045612 |
| | | | Flakes Freq | | Nectarine, | |
| | | | | | Plum Freq | |
| 4-methyl-2- | Egg Recipes | 0.073025 | SF_Beef_wt | 0.057631 | Beef, Veal, | 0.052266 |
| oxopentanoate | Freq | | | | Lamb, Pork, | |
| | | | | | Steak, Golash | |
| | | | | | Freq | |
| 1-myristoyl-2- | Cooked | −0.11559 | Tahini Salad | −0.07746 | SF_White | 0.067513 |
| palmitoyl-GPC | Legumes Freq | | Freq | | Cheese_wt | |
| (14:0/16:0) | | | | | | |
| arabonate/xylonate | SF_Coffee_wt | 0.150348 | Mandarin or | 0.065405 | Wholemeal or | 0.043615 |
| | | | Clementine | | Rye Bread | |
| | | | Freq | | Freq | |
| leucine | Cooked | −0.085 | SF_Beef_wt | 0.043223 | SF_Omelette_wt | 0.040266 |
| | Cereal such as | | | | | |
| | Oatmeal | | | | | |
| | Porridge Freq | | | | | |
| 5alpha-androstan- | Beer Freq | 0.178786 | Fries Freq | 0.075916 | SF_Milk_wt | −0.07192 |
| 3beta,17beta- | | | | | | |
| diol disulfate | | | | | | |
| 3-methylxanthine | Milk or Dark | 0.179884 | SF_Coffee_wt | 0.123982 | Coffee Freq | 0.107916 |
| | Chocolate | | | | | |
| | Freq | | | | | |
| X - 16087 | Fish Cooked, | 0.081994 | SF_Dark | 0.070058 | SF_Hummus | 0.064971 |
| | Baked or | | Chocolate_wt | | Salad_wt | |
| | Grilled Freq | | | | | |
| 3-methyl-2- | Egg Recipes | 0.071551 | Beef, Veal, | 0.05501 | White or | −0.05454 |
| oxovalerate | Freq | | Lamb, Pork, | | Brown Sugar | |
| | | | Steak, Golash | | Freq | |
| | | | Freq | | | |
| 2-hydroxybutyrate/ | Fish Cooked, | 0.083833 | Simple | −0.06585 | Olives Freq | 0.063047 |
| 2-hydroxyisobutyrate | Baked or | | Cookies or | | | |
| | Grilled Freq | | Biscuits Freq | | | |
| ergothioneine | SF_Mushrooms_wt | 0.054552 | Yeast Cakes | −0.04754 | White or | −0.04292 |
| | | | and Cookies | | Brown Sugar | |
| | | | as Rogallach, | | Freq | |
| | | | Croissant or | | | |
| | | | Donut Freq | | | |
| 1-lignoceroyl-GPC | Fries Freq | −0.09709 | SF_Tahini_wt | 0.084528 | SF_Banana_wt | 0.073216 |
| (24:0) | | | | | | |
| linoleoylcarnitine | SF_Tahini_wt | 0.124663 | SF_WhiteWheat_g_wt | 0.088672 | Nuts, | 0.0619 |
| (C18:2)* | | | | | almonds, | |
| | | | | | pistachios | |
| | | | | | Freq | |
| N-acetylcarnosine | Beer Freq | 0.138963 | SF_WhiteWheat_g_wt | 0.098821 | SF_Hummus | 0.064194 |
| | | | | | Salad_wt | |
| N-trimethyl 5- | SF_Milk_wt | 0.158341 | SF_Natural | 0.108177 | Salty Cheese, | 0.058578 |
| aminovalerate | | | Yogurt_wt | | Tzfatit, | |
| | | | | | Bulgarian, | |
| | | | | | Brinza, | |
| | | | | | Medium Slice | |
| | | | | | Freq | |
| sphingomyelin | SF_Milk_wt | 0.093966 | 3% Milk Freq | 0.061361 | SF_Dark | −0.05679 |
| (d18:1/22:2, | | | | | Chocolate_wt | |
| d18:2/22:1, | | | | | | |
| d16:1/24:2)* | | | | | | |
| urea | 0-1.5% | 0.052525 | Pastrami or | 0.049891 | 5-9% Yellow | 0.049752 |
| | Natural | | Smoked Turkey | | Cheese Freq | |
| | Yogurt Freq | | Breast Freq | | | |
| 3-carboxy-4- | Fish Cooked, | 0.097277 | Roll or | −0.07205 | SF_Couscous_wt | −0.05875 |
| methyl-5- | Baked or | | Bageles Freq | | | |
| pentyl-2- | Grilled Freq | | | | | |
| furanpropionate | | | | | | |
| (3-CMPFP)** | | | | | | |
| Fibrinopeptide | SF_Bread_wt | −0.10601 | Beer Freq | −0.03725 | 3% Milk Freq | 0.01296 |
| A(7-16)* | | | | | | |
| 3-(4-hydroxy- | SF_WhiteWheat_g_wt | 0.072752 | Kiwi or | −0.05595 | SF_Sugar Free | −0.05294 |
| phenyl)lactate | | | Strawberries | | Gum_wt | |
| | | | Freq | | | |
| 1-(1-enyl- | Chicken or | 0.068906 | Beef, Veal, | 0.058324 | Chicken or | 0.056297 |
| palmitoyl)-2- | Turkey | | Lamb, Pork, | | Turkey With | |
| linoleoyl-GPE | Without Skin | | Steak, Golash | | Skin Freq | |
| (P-16:0/18:2)* | Freq | | Freq | | | |
| X - 24948 | Beer Freq | 0.136619 | SF_Coffee_wt | −0.08636 | Orange or | 0.047517 |
| | | | | | Grapefruit | |
| | | | | | Juice Freq | |
| 1-(1-enyl-stearoyl)- | Egg, Hard | 0.070487 | Beef, Veal, | 0.06215 | Processed | −0.05125 |
| 2-oleoyl-GPE | Boiled or Soft | | Lamb, Pork, | | Meat Free | |
| (P-18:0/18:1) | Freq | | Steak, Golash | | Products Freq | |
| | | | Freq | | | |
| 3-hydroxybutyryl- | Cauliflower or | 0.062891 | SF_Whipped | 0.056602 | SF_Olives_wt | 0.056434 |
| carnitine (1) | Broccoli Freq | | cream_wt | | | |
| X - 19183 | SF_Orange_wt | 0.172544 | Mandarin or | 0.0745 | SF_Mandarin_wt | 0.042371 |
| | | | Clementine | | | |
| | | | Freq | | | |
| X - 23659 | Small Burekas | −0.0857 | SF_Tomatoes_wt | 0.068558 | SF_Vegetable | 0.065828 |
| | Freq | | | | Salad_wt | |
| 7-methylurate | SF_Coffee_wt | 0.259783 | Coffee Freq | 0.156831 | Milk or Dark | 0.082805 |
| | | | | | Chocolate | |
| | | | | | Freq | |
| X - 24757 | SF_Coffee_wt | 0.31171 | Peach, | 0.055613 | Fried Fish | −0.05359 |
| | | | Nectarine, | | Freq | |
| | | | Plum Freq | | | |
| X - 24328 | Yeast Cakes | 0.086134 | Watermelon | −0.05613 | Egg Recipes | 0.055837 |
| | and Cookies | | Freq | | Freq | |
| | as Rogallach, | | | | | |
| | Croissant or | | | | | |
| | Donut Freq | | | | | |
| pregn steroid | Beer Freq | 0.119691 | SF_Coffee_wt | −0.0608 | Shish Kebab | 0.040348 |
| monosulfate | | | | | in Pita Bread | |
| C21H34O5S* | | | | | Freq | |
| ethyl | SF_Wine_wt | 0.120814 | Alcoholic | 0.035542 | SF_Beer_wt | 0.02615 |
| glucuronide | | | Drinks Freq | | | |
| 3-hydroxyhippurate | SF_Coffee_wt | 0.254046 | SF_WholeWheat_g_wt | 0.108662 | Coffee Freq | 0.061144 |
| sulfate | | | | | | |
| 7-methylxanthine | Milk or Dark | 0.137426 | Coffee Freq | 0.117867 | SF_Dark | 0.093571 |
| | Chocolate | | | | Chocolate_wt | |
| | Freq | | | | | |
| X - 18886 | Fries Freq | 0.171409 | Olives Freq | 0.088129 | SF_Wine_wt | 0.058571 |
| glycine | Falafel in Pita | 0.12458 | SF_Tomatoes_wt | −0.09125 | SF_WhiteWheat_g_wt | 0.067141 |
| conjugate of | Bread Freq | | | | | |
| C10H14O2 (1)* | | | | | | |
| caprate (10:0) | SF_Coffee_wt | 0.074579 | SF_Butter_wt | 0.063468 | Butter Freq | 0.056425 |
| dihydroferulic | SF_Coffee_wt | 0.290151 | Coffee Freq | 0.148282 | 3-5% Natural | −0.0903 |
| acid | | | | | Yogurt Freq | |
| X - 12306 | SF_Tomatoes_wt | 0.105789 | Dried Fruits | 0.089391 | Herbal Tea | 0.060606 |
| | | | Freq | | Freq | |
| leucylalanine | SF_Bread_wt | −0.0722 | SF_Omelette_wt | −0.041 | SF_Beef_wt | −0.03317 |
| N1-methylinosine | SF_Orange_wt | −0.13759 | Orange or | −0.04785 | SF_Yellow | 0.041202 |
| | | | Grapefruit | | Cheese_wt | |
| | | | Freq | | | |
| X - 12544 | SF_WholeWheat_g_wt | 0.168178 | Wholemeal or | 0.151852 | Pasta or | 0.07402 |
| | | | Rye Bread | | Flakes Freq | |
| | | | Freq | | | |
| androstenediol | Beer Freq | 0.180346 | SF_Coffee_wt | −0.06751 | SF_WhiteWheat_g_wt | 0.055027 |
| (3alpha,17alpha) | | | | | | |
| monosulfate (3) | | | | | | |
| argininate* | Cooked | 0.12675 | Carrots, Fresh | 0.040944 | SF_Almonds_wt | 0.039395 |
| | Legumes Freq | | or Cooked, | | | |
| | | | Carrot Juice | | | |
| | | | Freq | | | |
| ferulic acid 4- | SF_Coffee_wt | 0.178886 | SF_Wholemeal | 0.08723 | Coffee Freq | 0.068412 |
| sulfate | | | Bread_wt | | | |
| pregnen-diol | Beer Freq | 0.135676 | SF_Coffee_wt | −0.09444 | Fries Freq | 0.030136 |
| disulfate | | | | | | |
| C21H34O8S2* | | | | | | |
| N-acetyl-3- | Chicken or | 0.092063 | Chicken or | 0.084964 | SF_Omelette_wt | 0.067281 |
| methylhistidine* | Turkey | | Turkey With | | | |
| | Without Skin | | Skin Freq | | | |
| | Freq | | | | | |
| X - 17655 | SF_Tahini_wt | 0.202332 | Tahini Salad | 0.070704 | SF_Hummus | 0.06714 |
| | | | Freq | | Salad_wt | |
| X - 24693 | White or | −0.0901 | Yeast Cakes | −0.08164 | SF_Tahini_wt | 0.071629 |
| | Brown Sugar | | and Cookies | | | |
| | Freq | | as Rogallach, | | | |
| | | | Croissant or | | | |
| | | | Donut Freq | | | |
| S-methylmethionine | Lettuce Freq | 0.098783 | SF_Vegetable | 0.089156 | Red Pepper | 0.068482 |
| | | | Salad_wt | | Freq | |
| X - 23314 | SF_Orange_wt | 0.084577 | Mandarin or | 0.059914 | SF_Banana_wt | 0.045285 |
| | | | Clementine | | | |
| | | | Freq | | | |
| sphingomyelin | SF_Dark | −0.09798 | 3% Milk Freq | 0.073143 | SF_Milk_wt | 0.054922 |
| (d18:1/20:2, | Chocolate_wt | | | | | |
| d18:2/20:1, | | | | | | |
| d16:1/22:2)* | | | | | | |
| androstenediol | SF_Coffee_wt | −0.09061 | Beer Freq | 0.081526 | Sugar | 0.064238 |
| (3alpha,17alpha) | | | | | Sweetened | |
| monosulfate (2) | | | | | Chocolate | |
| | | | | | Milk Freq | |
| alpha-hydroxy- | Beer Freq | 0.039992 | Beef, Veal, | 0.028714 | SF_Beef_wt | 0.026511 |
| isocaproate | | | Lamb, Pork, | | | |
| | | | Steak, Golash | | | |
| | | | Freq | | | |
| X - 24473 | Nuts, | 0.106187 | SF_Almonds_wt | 0.076986 | SF_Dried | 0.060379 |
| | almonds, | | | | dates_wt | |
| | pistachios | | | | | |
| | Freq | | | | | |
| X - 24337 | SF_Potatoes_wt | 0.091924 | SF_Salmon_wt | −0.08841 | SF_Water_wt | −0.08513 |
| X - 21829 | SF_Butter_wt | 0.068232 | SF_Wine_wt | 0.066805 | SF_Tomatoes | −0.06357 |
| | | | | | wt | |
| X - 23780 | Red Pepper | 0.214012 | Kiwi or | −0.03662 | SF_Vegetable | 0.034627 |
| | Freq | | Strawberries | | Salad_wt | |
| | | | Freq | | | |
| deoxycarnitine | Beer Freq | 0.081061 | SF_Vegetable | 0.069293 | SF_WhiteWheat_g_wt | 0.061974 |
| | | | Salad_wt | | | |
| N,N,N-trimethyl- | SF_WhiteWheat_g_wt | 0.076917 | SF_Beer_wt | 0.050797 | Hummus | 0.049043 |
| alanylproline | | | | | Salad Freq | |
| betaine (TMAP) | | | | | | |
| Fibrinopeptide | Beer Freq | −0.05044 | SF_Bread_wt | −0.04958 | Cooked | −0.02234 |
| B (1-13)** | | | | | Legumes Freq | |
| stearoylcarnitine | SF_Butter_wt | 0.076214 | SF_Dark | 0.066223 | SF_Beef_wt | 0.047197 |
| (C18) | | | Chocolate_wt | | | |
| myristate (14:0) | Artificial | 0.047372 | SF_Tahini_wt | −0.04426 | SF_Butter_wt | 0.041539 |
| | Sweeteners | | | | | |
| | Freq | | | | | |
| histidine | SF_Milk_wt | 0.079817 | Cooked | 0.071422 | SF_WhiteWheat_g_wt | −0.07029 |
| | | | Tomatoes, | | | |
| | | | Tomato | | | |
| | | | Sauce, | | | |
| | | | Tomato Soup | | | |
| | | | Freq | | | |
| isovaleryl- | SF_WhiteWheat_g_wt | 0.073234 | Cooked | −0.06223 | Chicken or | 0.051086 |
| carnitine (C5) | | | Cereal such as | | Turkey With | |
| | | | Oatmeal | | Skin Freq | |
| | | | Porridge Freq | | | |
| X - 13431 | SF_Butter_wt | 0.076001 | Alcoholic | 0.066183 | Butter Freq | 0.05745 |
| | | | Drinks Freq | | | |
| X - 13255 | Coffee Freq | 0.224197 | SF_Coffee_wt | 0.191833 | SF_Wholemeal | 0.063629 |
| | | | | | Crackers_wt | |
| X - 21319 | Fries Freq | 0.083015 | Cucumber | −0.05797 | Falafel in Pita | 0.054902 |
| | | | Freq | | Bread Freq | |
| X - 13866 | Fish Cooked, | 0.07941 | Canned Tuna | 0.072571 | SF_Tahini_wt | −0.06274 |
| | Baked or | | or Tuna Salad | | | |
| | Grilled Freq | | Freq | | | |
| 3-methyl-2- | Beef, Veal, | 0.035937 | Olives Freq | 0.032186 | SF_Soda | 0.029835 |
| oxobutyrate | Lamb, Pork, | | | | water_wt | |
| | Steak, Golash | | | | | |
| | Freq | | | | | |
| X - 07765 | Pasta or | 0.094759 | SF_Olive | −0.09441 | SF_WhiteWheat_g_wt | 0.060015 |
| | Flakes Freq | | oil_wt | | | |
| X - 22509 | SF_Tahini_wt | 0.220874 | SF_Water_wt | 0.042218 | SF_Mayonnaise_wt | −0.03097 |
| 2,3-dihydroxy- | Regular Sodas | −0.07983 | SF_Butter_wt | −0.06396 | Mandarin or | 0.061099 |
| 2-methylbutyrate | with Sugar | | | | Clementine | |
| | Freq | | | | Freq | |
| ADpSGEGDFX | Beer Freq | −0.09051 | SF_Bread_wt | −0.07817 | Salty Cheese, | 0.0402 |
| AEGGGVR* | | | | | Tzfatit, | |
| | | | | | Bulgarian, | |
| | | | | | Brinza, Thin | |
| | | | | | Slice Freq | |
| 5alpha-androstan- | Beer Freq | 0.173493 | SF_Vegetable | 0.05951 | SF_Rice | −0.05417 |
| 3alpha,17alpha- | | | Salad_wt | | crackers_wt | |
| diol monosulfate | | | | | | |
| X - 24832 | Cooked | −0.08267 | SF_Omelette_wt | 0.066657 | SF_Carrots_w | −0.06113 |
| | Cereal such as | | | | t | |
| | Oatmeal | | | | | |
| | Porridge Freq | | | | | |
| carotene diol | Red Pepper | 0.086142 | SF_Vegetable | 0.047378 | Yeast Cakes | −0.04061 |
| (1) | Freq | | Salad_wt | | and Cookies | |
| | | | | | as Rogallach, | |
| | | | | | Croissant or | |
| | | | | | Donut Freq | |
| 2-methylserine | Apple Freq | 0.225169 | SF_Apple_wt | 0.172245 | SF_Schnitzel_wt | −0.0719 |
| N-methylhydroxy- | SF_Orange_wt | 0.160394 | Mandarin or | 0.097522 | Orange or | 0.060004 |
| proline** | | | Clementine | | Grapefruit | |
| | | | Freq | | Freq | |
| catechol | SF_Coffee_wt | 0.215832 | Coffee Freq | 0.093628 | SF_Rice | 0.032019 |
| glucuronide | | | | | crackers_wt | |
| 3-hydroxyhippurate | SF_Coffee_wt | 0.19393 | Thousand | −0.07451 | Coffee Freq | 0.07307 |
| | | | Island | | | |
| | | | Dressing, | | | |
| | | | Garlic | | | |
| | | | Dressing Freq | | | |
| X - 18899 | SF_Tahini_wt | 0.125705 | Tahini Salad | 0.100376 | White or | −0.06859 |
| | | | Freq | | Brown Sugar | |
| | | | | | Freq | |
| pregnenetriol | Beer Freq | 0.121398 | SF_Coffee_wt | −0.08837 | Honey, Jam, | −0.03605 |
| disulfate* | | | | | fruit syrup, | |
| | | | | | Maple syrup | |
| | | | | | Freq | |
| N-stearoyl- | 3% Milk Freq | 0.074516 | SF_Tahini_wt | −0.06365 | Artificial | 0.060883 |
| sphingosine | | | | | Sweeteners | |
| (d18:1/18:0)* | | | | | Freq | |
| 10-undecenoate | SF_Tahini_wt | 0.109543 | Tahini Salad | 0.089383 | SF_Wine_wt | 0.064351 |
| (11:1n1) | | | Freq | | | |
| X - 15503 | SF_Carrots_w−t | −0.07267 | Apple Freq | 0.063306 | Schnitzel | 0.060649 |
| | | | | | Turkey or | |
| | | | | | Chicken Freq | |
| 1-palmitoyl-2- | SF_Tahini_wt | −0.10304 | SF_White | 0.057898 | Beer Freq | −0.04463 |
| palmitoleoyl-GPC | | | Cheese_wt | | | |
| (16:0/16:1)* | | | | | | |
| X - 15486 | Falafel in Pita | 0.073077 | SF_WhiteWheat_g_wt | 0.068423 | Coated or | 0.050947 |
| | Bread Freq | | | | Stuffed | |
| | | | | | Cookies, | |
| | | | | | Waffles or | |
| | | | | | Biscuits Freq | |
| gamma-tocopherol/ | Chicken or | −0.08596 | SF_Tahini_wt | 0.082135 | Cooked | 0.079452 |
| beta-tocopherol | Turkey | | | | Legumes Freq | |
| | Without Skin | | | | | |
| | Freq | | | | | |
| sphingomyelin | Cooked | −0.07581 | SF_Milk_wt | 0.073206 | Sour Cream | 0.049569 |
| (d18:1/21:0, | Legumes Freq | | | | Freq | |
| d17:1/22:0, | | | | | | |
| d16:1/23:0)* | | | | | | |
| 1-(1-enyl- | SF_Bread_wt | 0.069892 | SF_Wholemeal | −0.05993 | Beef or | 0.057894 |
| palmitoyl)-GPE | | | Bread_wt | | Chicken Soup | |
| (P-16:0)* | | | | | Freq | |
| isobutyryl- | SF_Natural | 0.063163 | 5-9% Yellow | 0.05372 | SF_Cereals_wt | 0.053375 |
| carnitine (C4) | Yogurt_wt | | Cheese Freq | | | |
| X - 18901 | SF_Banana_wt | 0.06399 | Diet Soda | −0.05372 | SF_Mayonnaise_wt | −0.04222 |
| | | | Freq | | | |
| gamma- | SF_Tomatoes_wt | −0.09994 | SF_Bread_wt | 0.052591 | SF_Sugar Free | −0.04414 |
| glutamylglutamate | | | | | Gum_wt | |
| X - 15492 | SF_WhiteWheat_g_wt | 0.076599 | Fried Fish | 0.074998 | Peanuts Freq | 0.069423 |
| | | | Freq | | | |
| X - 16580 | SF_Tahini_wt | −0.08956 | Lettuce Freq | 0.086593 | Olives Freq | 0.063551 |
| sphingomyelin | SF_Dark | −0.06768 | Wholemeal or | 0.050004 | Beer Freq | −0.04815 |
| (d18:2/24:2)* | Chocolate_wt | | Rye Bread | | | |
| | | | Freq | | | |
| stearoyl | SF_Milk_wt | 0.067077 | 3% Milk Freq | 0.047581 | >=16% Yellow | 0.039688 |
| sphingomyelin | | | | | Cheese Freq | |
| (d18:1/18:0) | | | | | | |
| N-methyltaurine | SF_Watermelon_wt | −0.17449 | Onion Freq | 0.147793 | Falafel in Pita | 0.122295 |
| | | | | | Bread Freq | |
| lysine | Chicken or | 0.098944 | Artificial | 0.056876 | Beef or | 0.052718 |
| | Turkey | | Sweeteners | | Chicken Soup | |
| | Without Skin | | Freq | | Freq | |
| | Freq | | | | | |
| X - 17340 | SF_Hummus | 0.116257 | Peanuts Freq | 0.097322 | Fried Fish | 0.07388 |
| | Salad_wt | | | | Freq | |
| X - 13703 | Coffee Freq | 0.188466 | SF_Coffee_wt | 0.152114 | SF_Rice | 0.046852 |
| | | | | | crackers_wt | |
| X - 24706 | SF_Soymilk_wt | 0.06487 | Cooked | 0.017659 | Zucchini or | 0.016865 |
| | | | Legumes Freq | | Eggplant Freq | |
| X - 22716 | 3% Milk Freq | −0.10833 | Cooked | 0.08906 | 0.5-3% White | −0.08144 |
| | | | Legumes Freq | | Cheese, | |
| | | | | | Cottage Freq | |
| X - 14082 | SF_Coffee_wt | 0.156468 | Coffee Freq | 0.152021 | Fresh | 0.042167 |
| | | | | | Vegetable | |
| | | | | | Salad With | |
| | | | | | Dressing or | |
| | | | | | Oil Freq | |
| 4-allylphenol | Apple Freq | 0.116885 | SF_Apple_wt | 0.076744 | SF_Milk_wt | −0.06441 |
| sulfate | | | | | | |
| 1-oleoyl-2- | Fish Cooked, | 0.084036 | SF_WhiteWheat_g_wt | −0.05803 | Jachnun, | −0.03538 |
| docosahexaenoyl- | Baked or | | | | Mlawah, | |
| GPC (18:1/22:6)* | Grilled Freq | | | | Kubana, | |
| | | | | | Cigars Freq | |
| X - 17354 | SF_Tahini_wt | 0.160742 | SF_Natural | 0.039212 | SF_Apple_wt | 0.034765 |
| | | | Yogurt_wt | | | |
| 6-oxopiperidine- | SF_Egg_wt | 0.06615 | Artificial | 0.062186 | Sugar | −0.05349 |
| 2-carboxylate | | | Sweeteners | | Sweetened | |
| | | | Freq | | Chocolate | |
| | | | | | Milk Freq | |
| X - 18240 | SF_Coffee_wt | 0.187494 | Coffee Freq | 0.0935 | SF_Wine_wt | 0.046175 |
| theanine | Green Tea | 0.096144 | SF_Green | 0.095442 | Regular Tea | 0.077232 |
| | Freq | | Tea_wt | | Freq | |
| X - 24760 | SF_Coffee_wt | 0.280824 | SF_WholeWheat_g_wt | 0.101318 | SF_Wholemeal | 0.057419 |
| | | | | | Crackers_wt | |
| beta-hydroxyiso- | Cooked | −0.06808 | Egg Recipes | 0.059491 | Olives Freq | 0.04804 |
| valerate | Cereal such as | | Freq | | | |
| | Oatmeal | | | | | |
| | Porridge Freq | | | | | |
| dodecenedioate | Nuts, | 0.117334 | 3% Milk Freq | −0.10753 | SF_Walnuts_wt | 0.102202 |
| (C12:1-DC)* | almonds, | | | | | |
| | pistachios | | | | | |
| | Freq | | | | | |
| X - 11478 | Fries Freq | 0.09509 | Carrots, Fresh | −0.06526 | Falafel in Pita | 0.060008 |
| | | | or Cooked, | | Bread Freq | |
| | | | Carrot Juice | | | |
| | | | Freq | | | |
| X - 24736 | SF_Tahini_wt | 0.112295 | SF_WhiteWheat_g_wt | −0.07968 | SF_Brown | 0.075141 |
| | | | | | Rice_wt | |
| lactose | 3% Milk Freq | 0.225955 | SF_Coffee_wt | 0.199653 | Cooked | −0.06794 |
| | | | | | Legumes Freq | |
| 2-hydroxyoctanoate | 3% Milk Freq | −0.09954 | SF_Tahini_wt | 0.071947 | Chicken or | −0.06708 |
| | | | | | Turkey | |
| | | | | | Without Skin | |
| | | | | | Freq | |
| trans-4- | Sausages Freq | 0.0654 | SF_Beef_wt | 0.054309 | Beef, Veal, | 0.052938 |
| hydroxyproline | | | | | Lamb, Pork, | |
| | | | | | Steak, Golash | |
| | | | | | Freq | |
| X - 17351 | Mandarin or | 0.058162 | SF_Brown | −0.04934 | Zucchini or | 0.046388 |
| | Clementine | | Sugar_wt | | Eggplant Freq | |
| | Freq | | | | | |
| 1-methylnicotin- | SF_Water_wt | 0.091722 | SF_Salmon_wt | 0.055713 | Beef or | 0.055467 |
| amide | | | | | Chicken Soup | |
| | | | | | Freq | |
| acetoacetate | Fish Cooked, | 0.073927 | Pasta or | −0.06951 | Ordinary | −0.05612 |
| | Baked or | | Flakes Freq | | Bread or | |
| | Grilled Freq | | | | Challah Freq | |
| X - 23782 | Regular Sodas | −0.10444 | Fish Cooked, | 0.10004 | Coated or | −0.04626 |
| | with Sugar | | Baked or | | Stuffed | |
| | Freq | | Grilled Freq | | Cookies, | |
| | | | | | Waffles or | |
| | | | | | Biscuits Freq | |
| X - 12818 | SF_Wholemeal | 0.184703 | SF_Cereals_wt | 0.104268 | Lemon Freq | −0.09019 |
| | Bread_wt | | | | | |
| 10- nonadecenoate | SF_Soymilk_wt | −0.03908 | Regular Sodas | −0.03743 | Butter Freq | 0.035973 |
| (19:1n9) | | | with Sugar | | | |
| | | | Freq | | | |
| X - 14314 | SF_Coffee_wt | 0.054253 | SF_Bread_wt | 0.041986 | SF_Butter_wt | 0.036913 |
| X - 24544 | Beer Freq | 0.085933 | SF_Coffee_wt | −0.0784 | Fries Freq | 0.04965 |
| gamma-glutamyl- | Cooked | −0.06413 | SF_WhiteWheat_g_wt | 0.054748 | White or | −0.04477 |
| leucine | Cereal such as | | | | Brown Sugar | |
| | Oatmeal | | | | Freq | |
| | Porridge Freq | | | | | |
| glutaryl- | Beer Freq | 0.13809 | SF_Beer_wt | 0.055295 | SF_Potatoes_wt | 0.051997 |
| carnitine (C5-DC) | | | | | | |
| hydantoin-5- | SF_Wholemeal | 0.080184 | Processed | −0.0621 | SF_Cottage | 0.053608 |
| propionic acid | Bread_wt | | Meat Free | | cheese_wt | |
| | | | Products Freq | | | |
| X - 12543 | SF_Coffee_wt | 0.289918 | Regular Tea | −0.08174 | Thousand | −0.05793 |
| | | | Freq | | Island | |
| | | | | | Dressing, | |
| | | | | | Garlic | |
| | | | | | Dressing Freq | |
| X - 17337 | SF_Cottage | 0.064122 | Fish (not | 0.060858 | SF_Tomatoes | −0.05977 |
| | cheese_wt | | Tuna) Pickled, | | _wt | |
| | | | Dried, Smoked, | | | |
| | | | Canned Freq | | | |
| dodecanedioate | SF_Tahini_wt | −0.15098 | Tahini Salad | −0.0647 | Coffee Freq | 0.062105 |
| | | | Freq | | | |
| androstenediol | Beer Freq | 0.109059 | SF_Coffee_wt | −0.06242 | SF_WhiteWheat_g_wt | 0.048352 |
| (3beta,17beta) | | | | | | |
| monosulfate (1) | | | | | | |
| adipoylcarnitine | SF_Tahini_wt | −0.09092 | SF_Carrots_wt | −0.04623 | SF_Olives_wt | 0.04375 |
| (C6-DC) | | | | | | |
| pristanate | Butter Freq | 0.171721 | Simple | −0.0926 | Beef, Veal, | 0.082812 |
| | | | Cookies or | | Lamb, Pork, | |
| | | | Biscuits Freq | | Steak, Golash | |
| | | | | | Freq | |
| sphingomyelin | Hummus | −0.06595 | SF_Potatoes_wt | −0.05092 | SF_Milk_wt | 0.05023 |
| (d18:2/23:0, | Salad Freq | | | | | |
| d18:1/23:1, | | | | | | |
| d17:1/24:1)* | | | | | | |
| X - 24542 | SF_Coffee_wt | 0.14031 | Regular Tea | −0.08879 | Herbal Tea | 0.06994 |
| | | | Freq | | Freq | |
| X - 22475 | SF_Coffee_wt | 0.18452 | SF_WholeWheat_g_wt | 0.096449 | 3% Milk Freq | 0.068639 |
| alpha-hydroxyiso- | SF_Coffee_wt | −0.05921 | SF_WhiteWheat_g_wt | 0.049964 | Beer Freq | 0.040813 |
| valerate | | | | | | |
| myristoylcarnitine | Butter Freq | 0.0533 | Cooked | −0.05196 | Beef, Veal, | 0.0485 |
| (C14) | | | Legumes Freq | | Lamb, Pork, | |
| | | | | | Steak, Golash | |
| | | | | | Freq | |
| X - 21411 | SF_Tahini_wt | 0.128825 | 3% Milk Freq | −0.07513 | Chicken or | −0.06842 |
| | | | | | Turkey | |
| | | | | | Without Skin | |
| | | | | | Freq | |
| 1-(1-enyl- | SF_Bread_wt | 0.059334 | Beef or | 0.052217 | SF_Wholemeal | −0.05039 |
| oleoyl)-GPE | | | Chicken Soup | | Bread_wt | |
| (P-18:1)* | | | Freq | | | |
| Fibrinopeptide | SF_Bread_wt | −0.04915 | Hummus | −0.0239 | Avocado Freq | 0.015385 |
| A (4-15)** | | | Salad Freq | | | |
| X - 11640 | SF_Tahini_wt | 0.247584 | SF_Water_wt | 0.059533 | SF_Red | −0.05334 |
| | | | | | pepper_wt | |
| 2-hydroxy-3- | Beer Freq | 0.049638 | SF_Milk_wt | −0.04903 | Herbal Tea | −0.04318 |
| methylvalerate | | | | | Freq | |
| dehydroiso- | Beer Freq | 0.14187 | SF_Coffee_wt | −0.08914 | Shish Kebab | 0.052195 |
| androsterone | | | | | in Pita Bread | |
| sulfate (DHEA-S) | | | | | Freq | |
| X - 12726 | SF_Coffee_wt | 0.092904 | SF_Vegetable | 0.072989 | SF_Olives_wt | 0.06468 |
| | | | Salad_wt | | | |
| X - 13728 | Milk or Dark | 0.153607 | SF_Dark | 0.087974 | Beef or | −0.04863 |
| | Chocolate | | Chocolate_wt | | Chicken Soup | |
| | Freq | | | | Freq | |
| cinnamoylglycine | SF_Coffee_wt | 0.126319 | Regular Sodas | −0.06715 | SF_Mayonnaise_wt | −0.06438 |
| | | | with Sugar | | | |
| | | | Freq | | | |
| X - 17685 | SF_Coffee_wt | 0.138281 | SF_WholeWheat_g_wt | 0.121406 | SF_Wine_wt | 0.065703 |
| X - 12101 | Brussels | 0.051094 | Cooked | 0.042104 | Falafel in Pita | 0.028582 |
| | Sprouts, | | Legumes Freq | | Bread Freq | |
| | Green or Red | | | | | |
| | Cabbage Freq | | | | | |
| glycocholenate | SF_Coffee_wt | −0.06995 | Coffee Freq | −0.0618 | SF_WhiteWheat_g_wt | 0.056011 |
| sulfate* | | | | | | |
| 4-hydroxyphenyl | SF_Cappuccino_wt | 0.049382 | SF_Fried | −0.04463 | SF_Bread_wt | −0.0354 |
| pyruvate | | | eggplant_wt | | | |
| 1-(1-enyl-palmitoyl)- | SF_WhiteWheat_g_wt | −0.10134 | SF_Hummus | −0.09715 | SF_Potatoes_wt | −0.04524 |
| 2-oleoyl-GPC | | | Salad_wt | | | |
| (P-16:0/18:1)* | | | | | | |
| picolinoylglycine | White or | −0.09267 | Processed | −0.05389 | SF_Tea_wt | −0.04419 |
| | Brown Sugar | | Meat Free | | | |
| | Freq | | Products Freq | | | |
| isocitrate | Red Pepper | 0.076783 | Pastrami or | −0.06361 | SF_Beef_wt | −0.05482 |
| | Freq | | Smoked Turkey | | | |
| | | | Breast Freq | | | |
| X - 24243 | SF_Cooked | −0.06315 | Pastrami or | 0.061746 | Turkey | 0.039993 |
| | beets_wt | | Smoked Turkey | | Meatballs, | |
| | | | Breast Freq | | Beef, Chicken | |
| | | | | | Freq | |
| androstenediol | Beer Freq | 0.134934 | SF_Coffee_wt | −0.08313 | Egg Recipes | 0.068449 |
| (3beta,17beta) | | | | | Freq | |
| disulfate (2) | | | | | | |
| X - 11261 | Falafel in Pita | 0.068496 | SF_Water_wt | −0.0405 | SF_Tomatoes_wt | −0.03592 |
| | Bread Freq | | | | | |
| X - 22162 | Orange or | 0.077606 | SF_Rice_wt | 0.052965 | SF_Hummus_wt | 0.052656 |
| | Grapefruit | | | | | |
| | Freq | | | | | |
| X - 11470 | Hummus | 0.100684 | SF_Rice | −0.08934 | Cucumber | −0.0614 |
| | Salad Freq | | crackers_wt | | Freq | |
| 2-methylbutyryl | SF_WhiteWheat_g_wt | 0.065268 | Cooked | −0.0465 | Dried Fruits | −0.03871 |
| carnitine (C5) | | | Cereal such as | | Freq | |
| | | | Oatmeal | | | |
| | | | Porridge Freq | | | |
| X - 12798 | 3% Milk Freq | 0.091414 | SF_Milk_wt | 0.074018 | SF_Tahini_wt | −0.04342 |
| dimethyl sulfoxide | SF_Tomatoes_wt | 0.05062 | SF_Potatoes_wt | −0.04519 | Red Pepper | 0.044749 |
| (DMSO) | | | | | Freq | |
| 2-aminooctanoate | SF_Tahini_wt | 0.105905 | 3% Milk Freq | −0.0914 | Beer Freq | 0.058881 |
| pentadecanoate | Butter Freq | 0.053376 | Olives Freq | 0.039644 | Cooked | −0.03932 |
| (15:0) | | | | | Legumes Freq | |
| 1,2-dilinoleoyl- | SF_Tahini_wt | 0.096569 | SF_Potatoes_wt | −0.06019 | Cooked | 0.055795 |
| GPC (18:2/18:2) | | | | | Legumes Freq | |
| X - 18921 | Fries Freq | 0.05387 | Coated or | 0.039743 | SF_WhiteWheat_g_wt | 0.03824 |
| | | | Stuffed | | | |
| | | | Cookies, | | | |
| | | | Waffles or | | | |
| | | | Biscuits Freq | | | |
| 1,2,3-benzenetriol | SF_Coffee_wt | 0.134102 | Coffee Freq | 0.052842 | SF_WholeWheat_g_wt | 0.043487 |
| sulfate (2) | | | | | | |
| nonadecanoate | Simple | −0.06058 | Butter Freq | 0.047339 | Coated or | −0.02794 |
| (19:0) | Cookies or | | | | Stuffed | |
| | Biscuits Freq | | | | Cookies, | |
| | | | | | Waffles or | |
| | | | | | Biscuits Freq | |
| gentisic acid- | 3% Milk Freq | −0.05801 | SF_Wholemeal | 0.047284 | Cauliflower or | 0.038263 |
| 5-glucoside | | | Bread_wt | | Broccoli Freq | |
| X - 18606 | SF_Tahini_wt | 0.142344 | SF_Hummus | 0.072401 | Beer Freq | 0.058927 |
| | | | Salad_wt | | | |
| hydroxy-N6,N6,N6- | SF_Sugar Free | −0.07963 | SF_Milk_wt | −0.07081 | SF_Vegetable | 0.064225 |
| trimethyllysine* | Gum_wt | | | | Salad_wt | |
| 3-(3-hydroxy- | SF_Coffee_wt | 0.227788 | Coffee Freq | 0.05958 | SF_WholeWheat_g_wt | 0.053186 |
| phenyl)propionate | | | | | | |
| sulfate | | | | | | |
| cytosine | SF_Wholemeal | 0.159775 | SF_Fried | −0.0547 | SF_WholeWheat_g_wt | 0.040952 |
| | Bread_wt | | onions_wt | | | |
| 2-hydroxynervonate* | Yeast Cakes | −0.0553 | SF_Noodles_wt | −0.04438 | SF_WhiteWheat_g_wt | −0.04422 |
| | and Cookies | | | | | |
| | as Rogallach, | | | | | |
| | Croissant or | | | | | |
| | Donut Freq | | | | | |
| 1-(1-enyl-stearoyl)- | Egg, Hard | 0.07593 | Beef or | 0.065637 | SF_Tahini_wt | 0.063931 |
| 2-linoleoyl-GPE | Boiled or Soft | | Chicken Soup | | | |
| (P-18:0/18:2)* | Freq | | Freq | | | |
| 1-palmitoyl-2- | Fish Cooked, | 0.057094 | SF_Wholemeal | 0.055869 | SF_Tahini_wt | −0.03623 |
| docosahexaenoyl-GPE | Baked or | | Light | | | |
| (16:0/22:6)* | Grilled Freq | | Bread_wt | | | |
| ADSGEGDFXAE | Orange or | −0.10373 | Salty Snacks | −0.08455 | Alcoholic | −0.07739 |
| GGGVR* | Grapefruit | | Freq | | Drinks Freq | |
| | Juice Freq | | | | | |
| 3-(3-hydroxyphenyl) | SF_Coffee_wt | 0.201776 | Coffee Freq | 0.052656 | SF_WholeWheat_g_wt | 0.038157 |
| propionate | | | | | | |
| N-stearoyltaurine | Beef, Veal, | 0.110496 | SF_Tomatoes_wt | −0.07775 | SF_Olives_wt | 0.042842 |
| | Lamb, Pork, | | | | | |
| | Steak, Golash | | | | | |
| | Freq | | | | | |
| 4-vinylphenol | Roll or | −0.1048 | SF_Coffee_wt | 0.10402 | Granola or | 0.065599 |
| sulfate | Bageles Freq | | | | Bernflaks | |
| | | | | | Freq | |
| N-acetyltaurine | SF_WhiteWheat_g_wt | 0.077176 | SF_Potatoes_wt | 0.065368 | Falafel in Pita | 0.036458 |
| | | | | | Bread Freq | |
| X - 24293 | Beer Freq | 0.091094 | SF_Wine_wt | 0.08129 | SF_WhiteWheat_g_wt | 0.07415 |
| tartronate | Red Pepper | 0.07617 | Turkey | −0.04034 | Vegetable | 0.037986 |
| (hydroxymalonate) | Freq | | Meatballs, | | Soup Freq | |
| | | | Beef, Chicken | | | |
| | | | Freq | | | |
| X - 22143 | Shish Kebab | 0.053932 | Sweet Dry | 0.044043 | Herbal Tea | −0.03614 |
| | in Pita Bread | | Wine, | | Freq | |
| | Freq | | Cocktails Freq | | | |
| pyrraline | SF_WholeWheat_g_wt | 0.061848 | Simple | 0.060612 | SF_Salmon_wt | −0.04986 |
| | | | Cookies or | | | |
| | | | Biscuits Freq | | | |
| 5-oxoproline | SF_Bread_wt | 0.036501 | Beer Freq | 0.02624 | SF_White | 0.025292 |
| | | | | | beans_wt | |
| margarate (17:0) | Simple | −0.03928 | Regular Sodas | −0.02758 | SF_Butter_wt | 0.025446 |
| | Cookies or | | with Sugar | | | |
| | Biscuits Freq | | Freq | | | |
| aconitate [cis | SF_Carrots_wt | −0.05062 | Potatoes | −0.04769 | Salty Snacks | −0.04748 |
| or trans] | | | Boiled, | | Freq | |
| | | | Baked, | | | |
| | | | Mashed, | | | |
| | | | Potatoes | | | |
| | | | Salad Freq | | | |
| 3,7-dimethylurate | Milk or Dark | 0.189726 | SF_Dark | 0.074185 | Beef or | −0.06911 |
| | Chocolate | | Chocolate_wt | | Chicken Soup | |
| | Freq | | | | Freq | |
| 1-stearoyl-2- | Fish Cooked, | 0.091596 | SF_Wholemeal | 0.054854 | SF_Tahini_wt | −0.04813 |
| docosahexaenoyl-GPE | Baked or | | Light | | | |
| (18:0/22:6)* | Grilled Freq | | Bread_wt | | | |
| X - 24801 | SF_WholeWheat_g_wt | 0.06358 | SF_Sugar Free | −0.05639 | Peanuts Freq | 0.049639 |
| | | | Gum_wt | | | |
| chiro-inositol | SF_Orange_wt | 0.107262 | Mandarin or | 0.051525 | Orange or | 0.048499 |
| | | | Clementine | | Grapefruit | |
| | | | Freq | | Freq | |
| trimethylamine | Roll or | −0.07952 | SF_Coffee_wt | 0.077516 | Processed | −0.05468 |
| N-oxide | Bageles Freq | | | | Meat Free | |
| | | | | | Products Freq | |
| 3-phenylpropionate | SF_Coffee_wt | 0.104958 | SF_Mayonnaise_wt | −0.1009 | SF_Apple_wt | 0.055215 |
| (hydrocinnamate) | | | | | | |
| X - 12283 | Egg Recipes | −0.05075 | SF_Hummus_wt | 0.050204 | Mandarin or | 0.050203 |
| | Freq | | | | Clementine | |
| | | | | | Freq | |
| X - 21410 | SF_Tahini_wt | −0.19852 | SF_Egg_wt | 0.191018 | SF_Beef_wt | 0.106045 |
| vanillyl- | Apple Freq | 0.077743 | Onion Freq | −0.06802 | SF_Banana_wt | 0.054575 |
| mandelate (VMA) | | | | | | |
| N-acetylglycine | SF_WhiteWheat_g_wt | −0.09448 | Herbal Tea | 0.059217 | Nuts, | 0.048257 |
| | | | Freq | | almonds, | |
| | | | | | pistachios | |
| | | | | | Freq | |
| X - 12812 | Mandarin or | 0.074454 | Orange or | 0.072369 | SF_Tomatoes_wt | 0.052836 |
| | Clementine | | Grapefruit | | | |
| | Freq | | Freq | | | |
| glycohyocholate | 3% Milk Freq | −0.09652 | SF_Beef_wt | −0.05554 | 0.5-3% White | −0.04343 |
| | | | | | Cheese, | |
| | | | | | Cottage Freq | |
| palmitoyl | Nuts, | 0.10047 | SF_WhiteWheat_g_wt | −0.06178 | SF_Potatoes_wt | −0.05827 |
| dihydrosphingo- | almonds, | | | | | |
| myelin | pistachios | | | | | |
| (d18:0/16:0)* | Freq | | | | | |
| gamma-CEHC | Pasta or | 0.107936 | Tahini Salad | −0.0595 | SF_Tahini_wt | −0.05082 |
| | Flakes Freq | | Freq | | | |
| X - 12472 | SF_Coffee_wt | 0.094319 | Falafel in Pita | 0.090484 | SF_Carrots_wt | −0.05648 |
| | | | Bread Freq | | | |
| 4-hydroxychloro- | SF_Cottage | 0.055377 | SF_Milk_wt | 0.048346 | Red Pepper | 0.045907 |
| thalonil | cheese_wt | | | | Freq | |
| 10-heptadecenoate | Regular Sodas | −0.04974 | Simple | −0.04505 | Artificial | 0.021249 |
| (17:1n7) | with Sugar | | Cookies or | | Sweeteners | |
| | Freq | | Biscuits Freq | | Freq | |
| X - 23644 | Red Pepper | 0.093291 | SF_Vegetable | 0.039215 | SF_Red | 0.033965 |
| | Freq | | Salad_wt | | pepper_wt | |
| X - 21821 | Mandarin or | 0.051696 | Apple Freq | 0.050414 | Zucchini or | 0.03914 |
| | Clementine | | | | Eggplant Freq | |
| | Freq | | | | | |
| X - 11444 | SF_Vegetable | 0.092755 | SF_Hummus | 0.058934 | SF_Carrots_wt | −0.05091 |
| | Salad_wt | | Salad_wt | | | |
| docosahexaenoyl- | Fish Cooked, | 0.109538 | SF_Salmon_wt | 0.076422 | SF_Bread_wt | 0.03848 |
| choline | Baked or | | | | | |
| | Grilled Freq | | | | | |
| gamma-glutamyl- | Beer Freq | 0.061011 | SF_Tomatoes_wt | −0.05921 | 0.5-3% White | −0.04567 |
| glutamine | | | | | Cheese, | |
| | | | | | Cottage Freq | |
| valine | White or | −0.05934 | Fish (not | 0.054359 | SF_Omelette_wt | 0.049183 |
| | Brown Sugar | | Tuna) Pickled, | | | |
| | Freq | | Dried, Smoked, | | | |
| | | | Canned Freq | | | |
| X - 13723 | SF_Coffee_wt | 0.193624 | Coffee Freq | 0.127054 | Vegetable | 0.03529 |
| | | | | | Soup Freq | |
| indolepropionate | Cooked | 0.046646 | Dried Fruits | 0.045609 | Wholemeal or | 0.043607 |
| | Legumes Freq | | Freq | | Rye Bread | |
| | | | | | Freq | |
| arabitol/xylitol | SF_Coffee_wt | 0.139771 | SF_Rice | 0.076099 | SF_Wholemeal | 0.066292 |
| | | | crackers_wt | | Bread_wt | |
| carnitine | Shish Kebab | 0.097013 | Chicken or | 0.061211 | SF_Vegetable | 0.046594 |
| | in Pita Bread | | Turkey With | | Salad_wt | |
| | Freq | | Skin Freq | | | |
| benzoylcarnitine* | SF_Coffee_wt | 0.157067 | SF_Apple_wt | 0.072915 | Coffee Freq | 0.071059 |
| X - 13729 | Onion Freq | −0.09022 | SF_Coffee_wt | 0.073443 | >=16% Yellow | 0.065813 |
| | | | | | Cheese Freq | |
| X - 12739 | Falafel in Pita | 0.071968 | Hummus | 0.069679 | SF_Hummus | 0.059622 |
| | Bread Freq | | Salad Freq | | Salad_wt | |
| 9-hydroxystearate | SF_Milk_wt | 0.095003 | Butter Freq | 0.068312 | Regular Sodas | −0.0617 |
| | | | | | with Sugar | |
| | | | | | Freq | |
| X - 21851 | SF_Apple_wt | −0.06377 | Sausages Freq | 0.049895 | Regular Sodas | 0.042147 |
| | | | | | with Sugar | |
| | | | | | Freq | |
| 13-methylmyristate | Sour Cream | 0.084894 | SF_Butter_wt | 0.083964 | SF_Milk_wt | 0.068971 |
| | Freq | | | | | |
| 7-ethylguanine | SF_Wine_wt | 0.066846 | Chicken or | −0.06106 | SF_WhiteWheat_g_wt | 0.05459 |
| | | | Turkey | | | |
| | | | Without Skin | | | |
| | | | Freq | | | |
| margaroylcarnitine* | Butter Freq | 0.105975 | >=16% Yellow | 0.097941 | Mixed Meat | 0.042884 |
| | | | Cheese Freq | | Dishes as | |
| | | | | | Moussaka, | |
| | | | | | Hamin, Cuba | |
| | | | | | Freq | |
| docosapentaenoate | Simple | −0.12007 | Apricot Fresh | 0.034649 | 0.5-3% White | 0.030771 |
| (n3 DPA; 22:5n3) | Cookies or | | or Dry, or | | Cheese, | |
| | Biscuits Freq | | Loquat Freq | | Cottage Freq | |
| X - 24546 | SF_Tahini_wt | −0.11829 | SF_Coffee_wt | −0.10119 | Tahini Salad | −0.07446 |
| | | | | | Freq | |
| X - 11787 | White or | −0.04505 | SF_Tomatoes_wt | −0.0379 | SF_Vegetable | 0.035632 |
| | Brown Sugar | | | | Salad_wt | |
| | Freq | | | | | |
| X - 24527 | SF_Hummus | 0.070502 | Falafel in Pita | 0.06244 | Hummus | 0.05344 |
| | Salad_wt | | Bread Freq | | Salad Freq | |
| 4-acetylphenol | SF_Coffee_wt | 0.156791 | 3% Milk Freq | −0.05685 | SF_Soymilk_wt | 0.049785 |
| sulfate | | | | | | |
| sphingomyelin | SF_Hummus | −0.06839 | Fresh | 0.053578 | SF_Milk_wt | 0.032785 |
| (d18:2/24:1, | Salad_wt | | Vegetable | | | |
| d18:1/24:2)* | | | Salad Without | | | |
| | | | Dressing or | | | |
| | | | Oil Freq | | | |
| cys-gly, oxidized | Pastrami or | 0.036794 | SF_Lettuce_wt | −0.03083 | Sausages Freq | 0.030657 |
| | Smoked Turkey | | | | | |
| | Breast Freq | | | | | |
| isoleucine | Cooked | −0.03656 | Herbal Tea | −0.03344 | SF_Tea_wt | −0.03212 |
| | Cereal such as | | Freq | | | |
| | Oatmeal | | | | | |
| | Porridge Freq | | | | | |
| cysteinylglycine | Pastrami or | 0.056683 | SF_Yellow | 0.054024 | SF_Sugar Free | −0.05318 |
| disulfide* | Smoked Turkey | | Cheese_wt | | Gum_wt | |
| | Breast Freq | | | | | |
| 1-myristoyl-2- | Cooked | −0.07118 | SF_White | 0.070052 | Onion Freq | 0.064567 |
| arachidonoyl-GPC | Legumes Freq | | Cheese_wt | | | |
| (14:0/20:4)* | | | | | | |
| 1-myristoyl- | Cooked | −0.11965 | Tahini Salad | −0.05657 | 3-5% Natural | 0.053361 |
| glycerol (14:0) | Legumes Freq | | Freq | | Yogurt Freq | |
| alpha-ketoglutarate | Artificial | 0.063637 | SF_Tomatoes_wt | −0.05738 | SF_Bread_wt | 0.047728 |
| | Sweeteners | | | | | |
| | Freq | | | | | |
| X - 24748 | SF_Tahini_wt | 0.17305 | 5-9% White | −0.06025 | Peanuts Freq | 0.060099 |
| | | | Cheese, | | | |
| | | | Cottage Freq | | | |
| eicosanodioate | SF_Hummus | 0.061637 | Alcoholic | 0.043798 | SF_Tahini_wt | 0.043589 |
| | Salad_wt | | Drinks Freq | | | |
| X - 24556 | SF_Tahini_wt | −0.15025 | Tahini Salad | −0.06057 | Cooked | −0.05647 |
| | | | Freq | | Legumes Freq | |
| X - 23680 | Falafel in Pita | 0.087917 | SF_WhiteWheat g_wt | 0.054224 | SF_Tomatoes_wt | −0.03333 |
| | Bread Freq | | | | | |
| acetylcarnitine | Chicken or | 0.064778 | Olives Freq | 0.054727 | Cauliflower or | 0.048316 |
| (C2) | Turkey With | | | | Broccoli Freq | |
| | Skin Freq | | | | | |
| hexanoylglutamine | SF_Olives_wt | 0.071153 | SF_Carrots_wt | −0.05522 | SF_Butter_wt | 0.040206 |
| sphingomyelin | SF_Hummus | −0.04922 | Beer Freq | −0.04036 | SF_Milk_wt | 0.037815 |
| (d18:1/18:1, | Salad_wt | | | | | |
| d18:2/18:0) | | | | | | |
| sphingomyelin | SF_Milk_wt | 0.061398 | Coffee Freq | 0.054795 | Apple Freq | −0.05461 |
| (d18:1/20:0, | | | | | | |
| d16:1/22:0)* | | | | | | |
| X - 23974 | Pasta or | −0.09086 | Hummus | −0.05679 | Lettuce Freq | 0.046852 |
| | Flakes Freq | | Salad Freq | | | |
| X - 12212 | Corn Freq | 0.071692 | SF_Milk_wt | −0.0674 | SF_White | 0.064989 |
| | | | | | Cheese_wt | |
| myristoleate | Regular Sodas | −0.0642 | SF_Soymilk_wt | −0.03688 | SF_Milk_wt | 0.036841 |
| (14:1n5) | with Sugar | | | | | |
| | Freq | | | | | |
| X - 13846 | Coffee Freq | 0.167835 | SF_Coffee_wt | 0.13385 | SF_Avocado_wt | −0.03013 |
| X - 21657 | SF_Milk_wt | −0.07147 | SF_Onion_wt | 0.068077 | Ice Cream or | −0.05106 |
| | | | | | Popsicle which | |
| | | | | | contains | |
| | | | | | Dairy Freq | |
| X - 24352 | Nuts, | 0.091291 | SF_Almonds_wt | 0.049197 | SF_Milk_wt | −0.03754 |
| | almonds, | | | | | |
| | pistachios | | | | | |
| | Freq | | | | | |
| beta- | SF_Bread_wt | 0.053222 | SF_Halva_wt | 0.044486 | SF_Hummus | −0.03712 |
| citrylglutamate | | | | | Salad_wt | |
| gluconate | Vegetable | 0.064582 | Mandarin or | 0.043157 | SF_Rice | 0.039038 |
| | Soup Freq | | Clementine | | crackers_wt | |
| | | | Freq | | | |
| lignoceroyl- | SF_Dark | 0.052863 | Light Bread | −0.05073 | SF_Couscous_wt | −0.0402 |
| carnitine (C24)* | Chocolate_wt | | Freq | | | |
| X - 24831 | Beef, Veal, | 0.037107 | SF_Carrots_wt | −0.03136 | Herbal Tea | −0.0304 |
| | Lamb, Pork, | | | | Freq | |
| | Steak, Golash | | | | | |
| | Freq | | | | | |
| Fibrinopeptide | SF_Bread_wt | −0.04056 | 3% Milk Freq | 0.027349 | Cauliflower or | 0.023617 |
| A (2-15)** | | | | | Broccoli Freq | |
| gamma-glutamyl- | Cooked | −0.05156 | SF_Sugar Free | −0.04448 | SF_Vegetable | 0.039899 |
| isoleucine* | Cereal such as | | Gum_wt | | Salad_wt | |
| | Oatmeal | | | | | |
| | Porridge Freq | | | | | |
| X - 12846 | Hummus | 0.080235 | Peanuts Freq | 0.077533 | SF_Vegetable | 0.055132 |
| | Salad Freq | | | | Salad_wt | |
| S-allylcysteine | SF_Hummus | 0.089282 | SF_Lettuce_wt | −0.05902 | SF_Cucumber_wt | −0.05122 |
| | Salad_wt | | | | | |
| tartarate | SF_Grapes_wt | 0.082038 | Turkey | −0.05606 | SF_Raisins_wt | 0.053596 |
| | | | Meatballs, | | | |
| | | | Beef, Chicken | | | |
| | | | Freq | | | |
| ceramide | Beef or | 0.091016 | SF_Tahini_wt | −0.07115 | 3% Milk Freq | 0.043787 |
| (d18:2/24:1, | Chicken Soup | | | | | |
| d18:1/24:2)* | Freq | | | | | |
| X - 12714 | SF_Coffee_wt | 0.154378 | SF_Salmon_wt | −0.06742 | Coffee Freq | 0.057035 |
| 1-stearoyl-2- | Beef, Veal, | −0.0542 | SF_WhiteWheat_g_wt | −0.02915 | 5-9% White | −0.02794 |
| linoleoyl-GPI | Lamb, Pork, | | | | Cheese, | |
| (18:0/18:2) | Steak, Golash | | | | Cottage Freq | |
| | Freq | | | | | |
| 1-linoleoyl- | Alcoholic | 0.134912 | SF_Potatoes_wt | −0.05422 | SF_Tahini_wt | 0.050325 |
| GPC (18:2) | Drinks Freq | | | | | |
| gamma-glutamyl- | SF_Vegetable | 0.047001 | SF_Wholemeal | 0.032299 | SF_Coffee_wt | 0.032297 |
| tyrosine | Salad_wt | | Bread_wt | | | |
| N-acetyl- | Coffee Freq | −0.05887 | 3% Milk Freq | −0.0416 | SF_Vegetable | 0.024152 |
| isoputreanine* | | | | | Salad_wt | |
| hexanoyl- | Chicken or | 0.047782 | Olives Freq | 0.045283 | Fresh | 0.038541 |
| carnitine (C6) | Turkey With | | | | Vegetable | |
| | Skin Freq | | | | Salad With | |
| | | | | | Dressing or | |
| | | | | | Oil Freq | |
| X - 16944 | Coated or | 0.05482 | SF_Tomatoes_wt | −0.03995 | Falafel in Pita | 0.038149 |
| | Stuffed | | | | Bread Freq | |
| | Cookies, | | | | | |
| | Waffles or | | | | | |
| | Biscuits Freq | | | | | |
| sucrose | SF_Cooked | −0.03833 | SF_Sugar | −0.03463 | SF_Water_wt | −0.0324 |
| | mushrooms_wt | | substitute_wt | | | |
| formimino- | Wholemeal or | −0.05219 | Dried Fruits | −0.05004 | Fish Cooked, | 0.043564 |
| glutamate | Rye Bread | | Freq | | Baked or | |
| | Freq | | | | Grilled Freq | |
| arachidoyl- | Light Bread | −0.1099 | SF_Tomatoes_wt | −0.07916 | SF_Couscous_wt | −0.07128 |
| carnitine (C20)* | Freq | | | | | |
| ximenoyl-carnitine | SF_Vegetable | 0.102459 | Lettuce Freq | 0.045823 | Avocado Freq | 0.044495 |
| (C26:1)* | Salad_wt | | | | | |
| hydroquinone | SF_Coffee_wt | 0.095988 | SF_Wholemeal | 0.0807 | SF_Cereals_wt | 0.048285 |
| sulfate | | | Bread_wt | | | |
| caprylate (8:0) | SF_Coffee_wt | 0.079323 | Carrots, Fresh | −0.06543 | SF_Yellow | 0.061268 |
| | | | or Cooked, | | Cheese_wt | |
| | | | Carrot Juice | | | |
| | | | Freq | | | |
| 3-methylcytidine | SF_WhiteWheat_g_wt | 0.090598 | SF_Coffee_wt | −0.07365 | Beer Freq | 0.069208 |
| riboflavin | SF_Natural | 0.090506 | 0.5-3% White | 0.070039 | SF_Coffee_wt | 0.04713 |
| (Vitamin B2) | Yogurt_wt | | Cheese, | | | |
| | | | Cottage Freq | | | |
| X - 14662 | SF_Apple_wt | −0.09769 | SF_Coffee_wt | −0.09098 | White or | 0.053462 |
| | | | | | Brown Sugar | |
| | | | | | Freq | |
| Fibrinopeptide | SF_Bread_wt | −0.04739 | Beer Freq | −0.02094 | Processed | −0.01091 |
| A(5-16)* | | | | | Meat Free | |
| | | | | | Products Freq | |
| X - 17335 | Pear Fresh, | −0.07045 | Nuts, | 0.06263 | Parsley, | 0.062528 |
| | Cooked or | | almonds, | | Celery, | |
| | Canned Freq | | pistachios | | Fennel, Dill, | |
| | | | Freq | | Cilantro, | |
| | | | | | Green Onion | |
| | | | | | Freq | |
| 3-hydroxy-3- | Apple Freq | 0.058912 | SF_Wholemeal | 0.057302 | Artificial | 0.039903 |
| methylglutarate | | | Bread_wt | | Sweeteners | |
| | | | | | Freq | |
| N-palmitoyl- | SF_Cucumber_wt | −0.11084 | >=16% Yellow | 0.10569 | Coffee Freq | 0.095998 |
| heptadeca- | | | Cheese Freq | | | |
| sphingosine | | | | | | |
| (d17:1/16:0)* | | | | | | |
| methyl-4- | SF_Potatoes_wt | −0.12041 | SF_Water_wt | 0.076849 | SF_WhiteWheat_g_wt | −0.03501 |
| hydroxybenzoate | | | | | | |
| sulfate | | | | | | |
| N-acetyl- | Pastrami or | 0.074605 | SF_Schnitzel_wt | 0.049129 | 3% Milk Freq | 0.03658 |
| cadaverine | Smoked Turkey | | | | | |
| | Breast Freq | | | | | |
| kynurenine | SF_Rice_wt | 0.051933 | 3-5% Natural | 0.041535 | SF_Coffee_wt | 0.036335 |
| | | | Yogurt Freq | | | |
| 5alpha-androstan- | Beer Freq | 0.089175 | Fries Freq | 0.066337 | SF_Beef_wt | 0.050897 |
| 3alpha,17beta-diol | | | | | | |
| monosulfate (1) | | | | | | |
| X - 21807 | SF_Wholemeal | 0.054191 | SF_Cucumber_wt | 0.049252 | SF_Granola_wt | 0.049025 |
| | Bread_wt | | | | | |
| X - 16946 | Red Pepper | −0.08129 | Beer Freq | 0.051737 | SF_Beer_wt | 0.047456 |
| | Freq | | | | | |
| X - 11485 | SF_Pickled | 0.109611 | Beer Freq | 0.060967 | Parsley, | 0.057116 |
| | cucumber_wt | | | | Celery, | |
| | | | | | Fennel, Dill, | |
| | | | | | Cilantro, | |
| | | | | | Green Onion | |
| | | | | | Freq | |
| methionine | Sugar | −0.04551 | SF_WhiteWheat_g_wt | −0.04156 | SF_Diet | −0.0375 |
| sulfone | Sweetened | | | | Coke_wt | |
| | Chocolate | | | | | |
| | Milk Freq | | | | | |
| 3-methoxycatechol | SF_Coffee_wt | 0.104167 | SF_WholeWheat_g_wt | 0.048877 | Coffee Freq | 0.0302 |
| sulfate (1) | | | | | | |
| N1-methyladenosine | SF_Cooked | −0.06155 | SF_Yellow | 0.045843 | Falafel in Pita | 0.045711 |
| | Sweet | | Cheese_wt | | Bread Freq | |
| | potato_wt | | | | | |
| andro steroid | SF_Tahini_wt | −0.1439 | 5-9% White | −0.06178 | SF_Coffee_wt | −0.05481 |
| monosulfate | | | Cheese, | | | |
| C19H28O6S (1)* | | | Cottage Freq | | | |
| X - 12712 | SF_Coffee_wt | 0.140458 | Banana Freq | −0.03517 | SF_Tahini_wt | 0.02072 |
| X - 21470 | SF_Coffee_wt | −0.11582 | Beer Freq | 0.096722 | Egg Recipes | 0.039388 |
| | | | | | Freq | |
| 1-oleoyl-2- | SF_Salmon_wt | 0.054117 | Couscous, | 0.046698 | SF_Onion_wt | −0.03554 |
| docosahexaenoyl- | | | Burgul, | | | |
| GPE (18:1/22:6)* | | | Mamaliga, | | | |
| | | | Groats Freq | | | |
| gamma-CEHC | SF_Tahini_wt | −0.12423 | Beer Freq | −0.06859 | SF_Schnitzel_wt | 0.033138 |
| glucuronide* | | | | | | |
| glycocholate | SF_Milk_wt | −0.04726 | Pastrami or | −0.04259 | 1% Milk Freq | −0.03616 |
| | | | Smoked Turkey | | | |
| | | | Breast Freq | | | |
| carboxyethyl-GABA | Pastrami or | −0.0866 | Sausages | −0.06704 | Cooked | 0.031391 |
| | Smoked Turkey | | such as | | Legumes Freq | |
| | Breast Freq | | Salami Freq | | | |
| N2,N2-dimethyl- | SF_Yellow | 0.098234 | Fried Fish | 0.066074 | SF_Sugar Free | −0.06334 |
| guanosine | Cheese_wt | | Freq | | Gum_wt | |
| X - 21310 | SF_Coffee_wt | 0.071409 | SF_Carrots_wt | −0.06521 | 5-9% White | 0.05211 |
| | | | | | Cheese, | |
| | | | | | Cottage Freq | |
| glycocheno- | SF_Coffee_wt | −0.0613 | Regular Tea | −0.04606 | 3% Milk Freq | −0.04532 |
| deoxycholate | | | Freq | | | |
| sulfate | | | | | | |
| N-acetyl-2- | SF_Tahini_wt | 0.137033 | Peanuts Freq | 0.059463 | SF_Milk_wt | −0.04204 |
| aminooctanoate* | | | | | | |
| X - 24410 | Coffee Freq | 0.087269 | SF_Water_wt | −0.05474 | Schnitzel | 0.039184 |
| | | | | | Turkey or | |
| | | | | | Chicken Freq | |
| 1-linoleoyl-2- | Beef, Veal, | −0.06829 | Nuts, | 0.032334 | SF_Omelette_wt | −0.02916 |
| linolenoyl-GPC | Lamb, Pork, | | almonds, | | | |
| (18:2/18:3)* | Steak, Golash | | pistachios | | | |
| | Freq | | Freq | | | |
| glycerophospho- | SF_Onion_wt | −0.07733 | Egg, Hard | 0.064763 | Hummus | −0.04904 |
| ethanolamine | | | Boiled or Soft | | Salad Freq | |
| | | | Freq | | | |
| X - 21792 | SF_Tahini_wt | −0.19733 | Tahini Salad | −0.10689 | Butter Freq | 0.061936 |
| | | | Freq | | | |
| 5-hydroxymethyl- | SF_Coffee_wt | 0.177999 | Coffee Freq | 0.077824 | SF_Tahini_wt | −0.04553 |
| 2-furoic acid | | | | | | |
| pipecolate | Brussels | 0.085255 | Butter Freq | −0.04196 | SF_Lentils_wt | 0.038667 |
| | Sprouts, | | | | | |
| | Green or Red | | | | | |
| | Cabbage Freq | | | | | |
| linoleoyl- | Nuts, | 0.058086 | SF_Hummus | 0.048527 | Butter Freq | −0.03714 |
| linoleoyl-glycerol | almonds, | | Salad_wt | | | |
| (18:2/18:2) [1]* | pistachios | | | | | |
| | Freq | | | | | |
| 3-hydroxy-2- | Simple | −0.05275 | Beef, Veal, | 0.045623 | SF_Yellow | 0.042843 |
| ethylpropionate | Cookies or | | Lamb, Pork, | | Cheese_wt | |
| | Biscuits Freq | | Steak, Golash | | | |
| | | | Freq | | | |
| 6-hydroxyindole | SF_Coffee_wt | 0.076349 | 5-9% White | 0.06381 | SF_Carrots_wt | −0.05916 |
| sulfate | | | Cheese, | | | |
| | | | Cottage Freq | | | |
| ectoine | Pastrami or | 0.084927 | Schnitzel | 0.044951 | SF_Chicken | 0.043943 |
| | Smoked Turkey | | Turkey or | | legs_wt | |
| | Breast Freq | | Chicken Freq | | | |
| 3-methyladipate | SF_WhiteWheat_g_wt | −0.09169 | White or | −0.07128 | SF_Apple_wt | 0.071033 |
| | | | Brown Sugar | | | |
| | | | Freq | | | |
| 3-hydroxyiso- | Dried Fruits | −0.05896 | SF_Cappuccino_wt | 0.050394 | SF_Natural | 0.049106 |
| butyrate | Freq | | | | Yogurt_wt | |
| 1-palmitoyl- | SF_Tahini_wt | −0.05885 | Pita Freq | −0.04177 | SF_Ice | 0.037925 |
| GPE (16:0) | | | | | cream_wt | |
| 1-palmitoyl-2- | SF_Tahini_wt | −0.10439 | SF_Hummus | −0.04759 | SF_Ice | 0.032992 |
| oleoyl-GPC | | | Salad_wt | | cream_wt | |
| (16:0/18:1) | | | | | | |
| laurate (12:0) | SF_Tahini_wt | −0.04977 | SF_Butter_wt | 0.035766 | Butter Freq | 0.033592 |
| X - 21441 | SF_Coffee_wt | −0.10118 | Green Tea | 0.05392 | Beer Freq | 0.040751 |
| | | | Freq | | | |
| X - 15674 | SF_Beef_wt | −0.08944 | Red Pepper | 0.069277 | SF_WhiteWheat_g_wt | −0.06332 |
| | | | Freq | | | |
| X - 21258 | SF_Wine_wt | 0.063455 | Alcoholic | 0.040641 | SF_Almonds_wt | 0.023999 |
| | | | Drinks Freq | | | |
| sulfate* | SF_Natural | 0.042297 | Pasta or | −0.04072 | SF_Coffee_wt | 0.028779 |
| | Yogurt_wt | | Flakes Freq | | | |
| docosahexaenoyl- | Fish Cooked, | 0.164689 | Fish (not | 0.056762 | SF_Beer_wt | 0.037362 |
| carnitine | Baked or | | Tuna) Pickled, | | | |
| (C22:6)* | Grilled Freq | | Dried, Smoked, | | | |
| | | | Canned Freq | | | |
| fumarate | SF_Bread_wt | 0.033833 | SF_Roll_wt | −0.02981 | Schnitzel | −0.02939 |
| | | | | | Turkey or | |
| | | | | | Chicken Freq | |
| propionylglycine | SF_Coffee_wt | 0.076691 | SF_Water_wt | 0.06895 | Egg, Hard | 0.040405 |
| | | | | | Boiled or Soft | |
| | | | | | Freq | |
| 1-ribosyl- | SF_Milk_wt | −0.05071 | Tahini Salad | 0.027727 | SF_Hummus_wt | 0.02567 |
| imidazoleacetate* | | | Freq | | | |
| 16a-hydroxy | SF_Tahini_wt | −0.07884 | SF_Coffee_wt | −0.07576 | Canned Tuna | 0.04132 |
| DHEA 3-sulfate | | | | | or Tuna Salad | |
| | | | | | Freq | |
| androstenediol | Beer Freq | 0.121029 | SF_WhiteWheat_g_wt | 0.079168 | SF_Coffee_wt | −0.04547 |
| (3beta,17beta) | | | | | | |
| disulfate (1) | | | | | | |
| pantothenate | Artificial | 0.072037 | SF_Tomatoes_wt | 0.032786 | Avocado Freq | 0.031684 |
| | Sweeteners | | | | | |
| | Freq | | | | | |
| X - 15461 | Chicken or | 0.044132 | Cooked | −0.03696 | SF_Coffee_wt | 0.031195 |
| | Turkey With | | Cereal such as | | | |
| | Skin Freq | | Oatmeal | | | |
| | | | Porridge Freq | | | |
| linoleoylcholine* | SF_Bread_wt | 0.050505 | Artificial | −0.04062 | SF_Tahini_wt | 0.038565 |
| | | | Sweeteners | | | |
| | | | Freq | | | |
| 1-linoleoyl- | Alcoholic | 0.061122 | Pastrami or | −0.05285 | SF_Walnuts_wt | 0.03184 |
| GPE (18:2)* | Drinks Freq | | Smoked Turkey | | | |
| | | | Breast Freq | | | |
| nisinate | SF_Tahini_wt | −0.09478 | Beer Freq | −0.06611 | 0.5-3% White | 0.062821 |
| (24:6n3) | | | | | Cheese, | |
| | | | | | Cottage Freq | |
| arachidate | Chicken or | −0.06088 | 3% Milk Freq | −0.05322 | Ordinary | −0.04074 |
| (20:0) | Turkey | | | | Bread or | |
| | Without Skin | | | | Challah Freq | |
| | Freq | | | | | |
| octadecenedioate | Regular Sodas | −0.05403 | Couscous, | 0.036199 | 3% Milk Freq | −0.0336 |
| (C18:1-DC)* | with Sugar | | Burgul, | | | |
| | Freq | | Mamaliga, | | | |
| | | | Groats Freq | | | |
| 1,2-dilinoleoyl-GPE | Cooked | 0.070297 | 3% Milk Freq | −0.05618 | SF_Coffee_wt | 0.049561 |
| (18:2/18:2)* | Legumes Freq | | | | | |
| acisoga | Coffee Freq | −0.0863 | Falafel in Pita | 0.059705 | SF_Tahini_wt | 0.030911 |
| | | | Bread Freq | | | |
| propionylcarnitine | Shish Kebab | 0.061404 | SF_Coffee_wt | 0.048186 | SF_Sugar Free | −0.04686 |
| (C3) | in Pita Bread | | | | Gum_wt | |
| | Freq | | | | | |
| 1-linoleoyl-GPG | SF_Water_wt | −0.04467 | SF_Natural | −0.03807 | SF_Soymilk_wt | 0.037041 |
| (18:2)* | | | Yogurt_wt | | | |
| X - 12263 | SF_Coffee_wt | 0.162606 | SF_Tomatoes_wt | −0.06167 | Coffee Freq | 0.053381 |
| X - 13553 | SF_Vegetable | 0.084191 | Cooked | −0.05454 | SF_Almonds_wt | 0.039301 |
| | Salad_wt | | Cereal such as | | | |
| | | | Oatmeal | | | |
| | | | Porridge Freq | | | |
| 5-hydroxyindole | Roll or | −0.04765 | SF_Almonds_wt | 0.046704 | Mandarin or | 0.04397 |
| acetate | Bageles Freq | | | | Clementine | |
| | | | | | Freq | |
| X - 21295 | SF_Coffee_wt | 0.140807 | SF_Wholemeal | 0.077468 | Banana Freq | −0.07549 |
| | | | Bread_wt | | | |
| Fibrinopeptide | SF_Bread_wt | −0.02982 | Beer Freq | −0.01324 | 3% Milk Freq | 0.00634 |
| A (3-16)** | | | | | | |
| N-palmitoyl- | SF_Coffee_wt | 0.051767 | SF_Tahini_wt | −0.04766 | SF_Pretzels_wt | −0.04221 |
| sphingosine | | | | | | |
| (d18:1/16:0) | | | | | | |
| X - 17677 | SF_Coffee_wt | 0.118219 | Coffee Freq | 0.065901 | SF_Wholemeal | 0.053262 |
| | | | | | Bread_wt | |
| 3-hydroxyhexanoate | SF_Carrots_wt | −0.0549 | Nuts, | 0.040265 | Olives Freq | 0.036866 |
| | | | almonds, | | | |
| | | | pistachios | | | |
| | | | Freq | | | |
| sphingomyelin | SF_Hummus | −0.06111 | Fresh | 0.049177 | Hummus | −0.04743 |
| (d18:1/24:1, | Salad_wt | | Vegetable | | Salad Freq | |
| d18:2/24:0)* | | | Salad Without | | | |
| | | | Dressing or | | | |
| | | | Oil Freq | | | |
| 1-carboxyethyl- | SF_Watermelon_wt | 0.043546 | SF_Burekas_wt | 0.042299 | 5-9% Yellow | 0.037741 |
| phenylalanine | | | | | Cheese Freq | |
| 3-hydroxy- | Wholemeal or | −0.03254 | Olives Freq | 0.031156 | Pear Fresh, | −0.02901 |
| butyrate (BHBA) | Rye Bread | | | | Cooked or | |
| | Freq | | | | Canned Freq | |
| X - 15469 | SF_Chocolate | −0.02756 | Olives Freq | 0.027483 | SF_Coffee_wt | −0.0258 |
| | cake_wt | | | | | |
| leucylglycine | SF_Chicken | −0.05293 | SF_Vegetable | −0.04372 | Processed | 0.040161 |
| | breast_wt | | Salad_wt | | Meat Free | |
| | | | | | Products Freq | |
| X - 23587 | Chicken or | 0.068007 | Fish (not | 0.066831 | Tomato Freq | −0.05204 |
| | Turkey With | | Tuna) Pickled, | | | |
| | Skin Freq | | Dried, Smoked, | | | |
| | | | Canned Freq | | | |
| gamma-glutamyl- | Banana Freq | −0.02702 | SF_Vegetable | 0.023917 | SF_Wholemeal | 0.021364 |
| phenylalanine | | | Salad_wt | | Bread_wt | |
| sphingomyelin | Sour Cream | 0.064972 | SF_Milk_wt | 0.056258 | Apple Freq | −0.05404 |
| (d18:1/22:1, | Freq | | | | | |
| d18:2/22:0, | | | | | | |
| d16:1/24:1)* | | | | | | |
| X - 24849 | Ordinary | 0.053709 | SF_Beer_wt | 0.042293 | Red Pepper | −0.03267 |
| | Bread or | | | | Freq | |
| | Challah Freq | | | | | |
| 1-stearoyl-2- | SF_Wholemeal | 0.064931 | SF_Tahini_wt | −0.02729 | SF_Onion_wt | −0.02352 |
| arachidonoyl-GPE | Light | | | | | |
| (18:0/20:4) | Bread_wt | | | | | |
| 17alpha-hydroxy- | SF_Coffee_wt | −0.07834 | SF_Lemon | 0.06481 | Beer Freq | 0.054433 |
| pregnenolone | | | juice_wt | | | |
| 3-sulfate | | | | | | |
| myo-inositol | Zucchini or | 0.056557 | Pasta or | −0.04226 | SF_Wine_wt | 0.034561 |
| | Eggplant Freq | | Flakes Freq | | | |
| 17alpha-hydroxy- | SF_Hummus | 0.106195 | SF_Beer_wt | 0.086303 | Artificial | −0.07952 |
| pregnanolone | Salad_wt | | | | Sweeteners | |
| glucuronide | | | | | Freq | |
| arachidonoyl- | SF_WhiteWheat_g_wt | 0.052902 | Hummus | 0.034309 | SF_Bread_wt | 0.033221 |
| carnitine | | | Salad Freq | | | |
| (C20:4) | | | | | | |
| stearidonate | Fish (not | 0.045552 | Mandarin or | 0.040018 | Canned Tuna | 0.034287 |
| (18:4n3) | Tuna) Pickled, | | Clementine | | or Tuna Salad | |
| | Dried, Smoked, | | Freq | | Freq | |
| | Canned Freq | | | | | |
| gamma-glutamyl- | Green Pepper | 0.039247 | SF_Cappuccino_wt | 0.030677 | Chicken or | 0.029409 |
| alpha-lysine | Freq | | | | Turkey | |
| | | | | | Without Skin | |
| | | | | | Freq | |
| 3-indoxyl sulfate | SF_Coffee_wt | 0.07973 | SF_Carrots_wt | −0.07335 | 5-9% White | 0.06247 |
| | | | | | Cheese, | |
| | | | | | Cottage Freq | |
| 1-stearoyl-2- | Nuts, | 0.05006 | SF_Tahini_wt | 0.043015 | SF_Potatoes_wt | −0.02857 |
| linoleoyl-GPC | almonds, | | | | | |
| (18:0/18:2)* | pistachios | | | | | |
| | Freq | | | | | |
| X - 17327 | SF_Yellow | 0.130344 | SF_Wholemeal | −0.06456 | Milk or Dark | −0.04598 |
| | Cheese_wt | | Bread_wt | | Chocolate | |
| | | | | | Freq | |
| 1-stearoyl-2- | SF_Dark | 0.050532 | Thousand | −0.0405 | SF_Tahini_wt | −0.02715 |
| oleoyl-GPC | Chocolate_wt | | Island | | | |
| (18:0/18:1) | | | Dressing, | | | |
| | | | Garlic | | | |
| | | | Dressing Freq | | | |
| 1-stearoyl-GPC | Nuts, | 0.048795 | SF_Tomatoes_wt | −0.03172 | Thousand | −0.01745 |
| (18:0) | almonds, | | | | Island | |
| | pistachios | | | | Dressing, | |
| | Freq | | | | Garlic | |
| | | | | | Dressing Freq | |
| X - 23593 | SF_Rice | 0.047886 | SF_Tomatoes_wt | 0.030574 | SF_Vegetable | 0.030164 |
| | crackers_wt | | | | Salad_wt | |
| 1-linoleoyl-GPI | Chicken or | −0.07118 | SF_Rice | 0.052388 | >=16% Yellow | −0.02918 |
| (18:2)* | Turkey | | crackers_wt | | Cheese Freq | |
| | Without Skin | | | | | |
| | Freq | | | | | |
| linolenate | Nuts, | 0.082233 | Chicken or | −0.03234 | Regular Sodas | −0.03105 |
| [alpha or gamma; | almonds, | | Turkey | | with Sugar | |
| (18:3n3 or 6)] | pistachios | | Without Skin | | Freq | |
| | Freq | | Freq | | | |
| glucuronate | Cooked | 0.053612 | Olives Freq | 0.048299 | SF_Tea_wt | −0.04478 |
| | Vegetable | | | | | |
| | Salads Freq | | | | | |
| cerotoylcarnitine | SF_Dark | 0.065789 | SF_Vegetable | 0.05387 | Beef, Veal, | 0.048883 |
| (C26)* | Chocolate_wt | | Salad_wt | | Lamb, Pork, | |
| | | | | | Steak, Golash | |
| | | | | | Freq | |
| alpha-tocopherol | Regular Sodas | −0.0752 | Zucchini or | 0.044517 | Pita Freq | −0.03676 |
| | with Sugar | | Eggplant Freq | | | |
| | Freq | | | | | |
| cystine | SF_White | 0.06269 | SF_Milk_wt | −0.05379 | Processed | −0.03521 |
| | Cheese_wt | | | | Meat Free | |
| | | | | | Products Freq | |
| vanillic alcohol | 3% Milk Freq | −0.06089 | Regular Tea | −0.05546 | Zucchini or | 0.05366 |
| sulfate | | | Freq | | Eggplant Freq | |
| palmitoleate | Regular Sodas | −0.05418 | Apricot Fresh | 0.022006 | Artificial | 0.018981 |
| (16:1n7) | with Sugar | | or Dry, or | | Sweeteners | |
| | Freq | | Loquat Freq | | Freq | |
| o-cresol sulfate | Coffee Freq | 0.095003 | Sugar | −0.0362 | Lemon Freq | 0.027052 |
| | | | Sweetened | | | |
| | | | Chocolate | | | |
| | | | Milk Freq | | | |
| 1-palmitoyl-2- | SF_Wholemeal | 0.046446 | SF_Dried | −0.04394 | SF_Tahini_wt | −0.04326 |
| arachidonoyl-GPC | Light | | dates_wt | | | |
| (16:0/20:4n6) | Bread_wt | | | | | |
| methylsuccinoyl- | SF_Hummus | −0.0525 | Cooked | −0.0439 | SF_Natural | 0.043251 |
| carnitine (1) | Salad_wt | | Tomatoes, | | Yogurt_wt | |
| | | | Tomato | | | |
| | | | Sauce, | | | |
| | | | Tomato Soup | | | |
| | | | Freq | | | |
| X - 24972 | SF_Egg_wt | −0.08948 | SF_Yellow | −0.05579 | SF_Butter_wt | −0.03895 |
| | | | Cheese_wt | | | |
| X - 23666 | SF_WhiteWheat_g_wt | 0.068366 | Sausages Freq | 0.041272 | Salty Snacks | 0.030829 |
| | | | | | Freq | |
| decanoylcarnitine | Olives Freq | 0.054851 | SF_Watermel on_wt | −0.03923 | 1% Milk Freq | −0.02785 |
| (C10) | | | | | | |
| X - 21353 | Nuts, | 0.059715 | Falafel in Pita | 0.05262 | SF_Tahini_wt | 0.041466 |
| | almonds, | | Bread Freq | | | |
| | pistachios | | | | | |
| | Freq | | | | | |
| etiocholanolone | Beer Freq | 0.060124 | SF_Onion_wt | 0.044929 | Sugar | 0.035631 |
| glucuronide | | | | | Sweetened | |
| | | | | | Chocolate | |
| | | | | | Milk Freq | |
| X - 17353 | SF_Sugar Free | 0.079919 | 5-9% White | 0.032881 | Cooked | 0.026736 |
| | Gum_wt | | Cheese, | | Cereal such as | |
| | | | Cottage Freq | | Oatmeal | |
| | | | | | Porridge Freq | |
| X - 24329 | Falafel in Pita | 0.052792 | 1% Milk Freq | −0.03275 | SF_Potatoes_wt | 0.026598 |
| | Bread Freq | | | | | |
| 2-arachidonoyl- | Regular Sodas | 0.090073 | SF_Rice_wt | 0.072147 | SF_WhiteWheat_g_wt | 0.052247 |
| glycerol (20:4) | with Sugar | | | | | |
| | Freq | | | | | |
| sarcosine | Egg, Hard | 0.061378 | SF_Omelette_wt | 0.043441 | SF_WhiteWheat_g_wt | 0.037801 |
| | Boiled or Soft | | | | | |
| | Freq | | | | | |
| alpha-ketobutyrate | Fish Cooked, | 0.089386 | SF_Tofu_wt | −0.05264 | Granola or | −0.04186 |
| | Baked or | | | | Bernflaks | |
| | Grilled Freq | | | | Freq | |
| citrate | SF_Lettuce_wt | −0.05888 | Light Bread | −0.05104 | SF_Carrots_wt | −0.0482 |
| | | | Freq | | | |
| pregnenolone | Beer Freq | 0.065808 | SF_Coffee_wt | −0.05694 | SF_Lemon | 0.036754 |
| sulfate | | | | | juice_wt | |
| eicosenoate | Yeast Cakes | −0.0319 | SF_Noodles_wt | −0.02962 | Simple | −0.0269 |
| (20:1) | and Cookies | | | | Cookies or | |
| | as Rogallach, | | | | Biscuits Freq | |
| | Croissant or | | | | | |
| | Donut Freq | | | | | |
| 5alpha-androstan- | Beer Freq | 0.089884 | Fries Freq | 0.070706 | SF_Pita_wt | 0.049846 |
| 3beta,17beta-diol | | | | | | |
| monosulfate (2) | | | | | | |
| hypotaurine | Cooked | 0.043805 | Processed | 0.039617 | SF_Cappuccino_wt | −0.03939 |
| | Legumes Freq | | Meat Free | | | |
| | | | Products Freq | | | |
| tauro-beta- | SF_Sugar Free | 0.073393 | Shish Kebab | −0.05382 | 3% Milk Freq | −0.04577 |
| muricholate | Gum_wt | | in Pita Bread | | | |
| | | | Freq | | | |
| eicosapentaenoyl- | SF_Tahini_wt | −0.12604 | Fish Cooked, | 0.106876 | SF_Salmon_wt | 0.088145 |
| choline | | | Baked or | | | |
| | | | Grilled Freq | | | |
| 1-oleoyl-GPE | 3% Milk Freq | −0.05434 | Pastrami or | −0.04671 | SF_Yellow | −0.02447 |
| (18:1) | | | Smoked Turkey | | Cheese_wt | |
| | | | Breast Freq | | | |
| 1-palmitoyl-2- | SF_Wholemeal | 0.067457 | SF_Tahini_wt | −0.04766 | Onion Freq | 0.037292 |
| arachidonoyl-GPE | Light | | | | | |
| (16:0/20:4)* | Bread_wt | | | | | |
| androsterone | Beer Freq | 0.089625 | Fries Freq | 0.027494 | SF_Coffee_wt | −0.02314 |
| sulfate | | | | | | |
| 2-acetamidophenol | SF_Wholemeal | 0.090403 | Granola or | 0.067783 | Cooked | 0.056602 |
| sulfate | Bread_wt | | Bernflaks | | Cereal such as | |
| | | | Freq | | Oatmeal | |
| | | | | | Porridge Freq | |
| X - 01911 | SF_Milk_wt | −0.0681 | Kiwi or | −0.05011 | Apricot Fresh | −0.04798 |
| | | | Strawberries | | or Dry, or | |
| | | | Freq | | Loquat Freq | |
| nicotinamide | SF_Bread_wt | 0.071635 | SF_Coffee_wt | −0.03908 | SF_Water_wt | 0.026042 |
| X - 11522 | Ordinary | 0.044847 | SF_Beer_wt | 0.031155 | SF_Rice_wt | 0.023121 |
| | Bread or | | | | | |
| | Challah Freq | | | | | |
| X - 12753 | SF_Onion_wt | 0.09065 | Milk or Dark | −0.05508 | SF_Bread_wt | −0.02556 |
| | | | Chocolate | | | |
| | | | Freq | | | |
| N-palmitoyl- | Coffee Freq | 0.096446 | Tomato Freq | −0.08704 | SF_Coffee_wt | 0.058111 |
| sphinganine | | | | | | |
| (d18:0/16:0) | | | | | | |
| X - 12844 | Fried Fish | 0.04782 | SF_Carrots_wt | −0.04441 | Milk or Dark | −0.03873 |
| | Freq | | | | Chocolate | |
| | | | | | Freq | |
| X - 12410 | SF_Banana_wt | 0.056995 | Orange or | −0.02864 | SF_Avocado_wt | 0.027351 |
| | | | Grapefruit | | | |
| | | | Juice Freq | | | |
| erucate | Fish (not | 0.107709 | Cauliflower or | 0.032695 | White or | −0.02819 |
| (22:1n9) | Tuna) Pickled, | | Broccoli Freq | | Brown Sugar | |
| | Dried, Smoked, | | | | Freq | |
| | Canned Freq | | | | | |
| X - 16964 | SF_Cranberries_wt | 0.101215 | SF_Vegetable | 0.075652 | SF_Yellow | −0.0591 |
| | | | Salad_wt | | Cheese_wt | |
| palmitoyl- | SF_Beef_wt | 0.046691 | Beef, Veal, | 0.044311 | SF_WhiteWheat_g_wt | 0.03838 |
| carnitine (C16) | | | Lamb, Pork, | | | |
| | | | Steak, Golash | | | |
| | | | Freq | | | |
| glyco-beta- | SF_Beef_wt | −0.06478 | Peas, Green | 0.063065 | 0.5-3% White | −0.04681 |
| muricholate** | | | Beans or Okra | | Cheese, | |
| | | | Cooked Freq | | Cottage Freq | |
| X - 21628 | Beer Freq | −0.06377 | SF_WhiteWheat_g_wt | −0.02921 | White or | −0.0239 |
| | | | | | Brown Sugar | |
| | | | | | Freq | |
| gamma- | SF_Tomatoes_wt | −0.05188 | SF_Tahini_wt | 0.034775 | Orange or | 0.030931 |
| glutamylglycine | | | | | Grapefruit | |
| | | | | | Freq | |
| kynurenate | SF_Vegetable | 0.051277 | 0-1.5% | 0.038151 | SF_Lentils_wt | −0.03395 |
| | Salad_wt | | Natural | | | |
| | | | Yogurt Freq | | | |
| proline | SF_WhiteWheat_g_wt | 0.071788 | 5-9% White | 0.039607 | SF_Lentils_wt | −0.03648 |
| | | | Cheese, | | | |
| | | | Cottage Freq | | | |
| X - 21285 | Beer Freq | 0.071763 | SF_Coffee_wt | −0.06569 | SF_Rice | −0.04881 |
| | | | | | crackers_wt | |
| 3-hydroxyoctanoate | Butter Freq | 0.099174 | Nuts, | 0.049921 | Carrots, Fresh | −0.04729 |
| | | | almonds, | | or Cooked, | |
| | | | pistachios | | Carrot Juice | |
| | | | Freq | | Freq | |
| N6,N6,N6- | SF_Vegetable | 0.067066 | Beer Freq | 0.031592 | SF_Omelette_wt | 0.030373 |
| trimethyllysine | Salad_wt | | | | | |
| phenylacetate | SF_WhiteWheat_g_wt | −0.07144 | SF_Mayonnaise_wt | −0.04729 | Onion Freq | −0.04418 |
| glutamine | Hummus | 0.033603 | Tahini Salad | 0.027003 | Beer Freq | 0.023909 |
| | Salad Freq | | Freq | | | |
| homocitrulline | SF_WhiteWheat_g_wt | −0.06447 | SF_Egg_wt | 0.055245 | SF_Natural | 0.048659 |
| | | | | | Yogurt_wt | |
| X - 21659 | SF_Milk_wt | −0.09211 | Onion Freq | 0.063348 | SF_Soda | 0.057046 |
| | | | | | water_wt | |
| N-acetyltyrosine | SF_Cappuccino_wt | 0.094836 | SF_Coffee_wt | 0.058616 | SF_Hummus_wt | −0.03856 |
| X - 21474 | SF_Milk_wt | −0.07991 | SF_Beer_wt | 0.0649 | SF_Pickled | 0.05981 |
| | | | | | cucumber_wt | |
| X - 12026 | Processed | −0.08968 | SF_Yellow | 0.076298 | SF_Carrots_wt | −0.03611 |
| | Meat Free | | Cheese_wt | | | |
| | Products Freq | | | | | |
| xylose | Nuts, | 0.098204 | 3% Milk Freq | −0.05523 | Beef, Veal, | −0.04884 |
| | almonds, | | | | Lamb, Pork, | |
| | pistachios | | | | Steak, Golash | |
| | Freq | | | | Freq | |
| dihomo-linolenoyl- | SF_WhiteWheat_g_wt | 0.041331 | SF_Bread_wt | 0.037511 | Lettuce Freq | −0.0354 |
| choline | | | | | | |
| X - 24106 | SF_Schnitzel_wt | −0.04488 | 5-9% Yellow | −0.03897 | SF_WholeWheat_g_wt | −0.03771 |
| | | | Cheese Freq | | | |
| X - 14095 | SF_Bread_wt | 0.077635 | SF_Hummus | −0.03217 | SF_Butter_wt | 0.025451 |
| | | | Salad_wt | | | |
| tyrosine | 5-9% Yellow | 0.027014 | 5-9% White | 0.022504 | SF_Wholemeal | 0.021459 |
| | Cheese Freq | | Cheese, | | Bread_wt | |
| | | | Cottage Freq | | | |
| dihomo-linoleoyl- | SF_Tahini_wt | 0.168036 | Turkey | −0.07754 | SF_Tomatoes_wt | −0.04487 |
| carnitine | | | Meatballs, | | | |
| (C20:2)* | | | Beef, Chicken | | | |
| | | | Freq | | | |
| asparagine | Cooked | 0.039672 | SF_Noodles_wt | 0.036839 | SF_Rice | 0.026258 |
| | Legumes Freq | | | | crackers_wt | |
| N-acetylmethionine | SF_Bread_wt | 0.011027 | SF_Roll_wt | −0.00322 | SF_Butter_wt | 0.002863 |
| X - 21364 | Beer Freq | 0.075149 | SF_Coffee_wt | −0.03726 | SF_Beer_wt | 0.033377 |
| X - 25116 | SF_Burekas_wt | 0.036163 | SF_Natural | −0.03369 | SF_Coffee_wt | −0.02773 |
| | | | Yogurt_wt | | | |
| 3beta- | Alcoholic | 0.077874 | SF_White | −0.03951 | SF_Salmon_wt | −0.03235 |
| hydroxy-5- | Drinks Freq | | Cheese_wt | | | |
| cholestenoate | | | | | | |
| dopamine 4- | SF_Banana_wt | 0.070655 | Sugar | −0.06142 | SF_Wholemeal | 0.050857 |
| sulfate | | | Sweetened | | Bread_wt | |
| | | | Chocolate | | | |
| | | | Milk Freq | | | |
| pyridoxate | Roll or | −0.07028 | Green Pepper | 0.056616 | Lettuce Freq | 0.051359 |
| | Bageles Freq | | Freq | | | |
| N-acetyl-1- | Beef, Veal, | 0.078307 | Beef or | 0.045135 | SF_Chicken | 0.04307 |
| methylhistidine* | Lamb, Pork, | | Chicken Soup | | legs_wt | |
| | Steak, Golash | | Freq | | | |
| | Freq | | | | | |
| guanidinoacetate | SF_Vegetable | 0.074438 | Tahini Salad | 0.040045 | Garlic Freq | −0.03967 |
| | Salad_wt | | Freq | | | |
| 21-hydroxy- | SF_Vegetable | −0.07337 | Fries Freq | 0.049259 | SF_Coffee_wt | −0.04457 |
| pregnenolone | Salad_wt | | | | | |
| disulfate | | | | | | |
| malate | SF_Bread_wt | 0.032741 | Light Bread | −0.02165 | SF_Butter_wt | 0.014048 |
| | | | Freq | | | |
| oleoylcarnitine | Olives Freq | 0.077644 | SF_Couscous_ wt | −0.03024 | SF_Ketchup_wt | −0.02392 |
| (C18:1) | | | | | | |
| X - 12206 | Red Pepper | 0.046815 | SF_Mandarin_wt | 0.043388 | Turkey | −0.03816 |
| | Freq | | | | Meatballs, | |
| | | | | | Beef, Chicken | |
| | | | | | Freq | |
| X - 12063 | SF_Sugar Free | −0.0584 | SF_WhiteWheat_g_wt | 0.054733 | Pastrami or | 0.033261 |
| | Gum_wt | | | | Smoked Turkey | |
| | | | | | Breast Freq | |
| oleoyl | White or | −0.03498 | Small Burekas | −0.03307 | Yeast Cakes | −0.0258 |
| ethanolamide | Brown Sugar | | Freq | | and Cookies | |
| | Freq | | | | as Rogallach, | |
| | | | | | Croissant or | |
| | | | | | Donut Freq | |
| glutamate | SF_Bread_wt | 0.036395 | Orange or | −0.01802 | SF_WhiteWheat_g_wt | 0.017001 |
| | | | Grapefruit | | | |
| | | | Freq | | | |
| phenylacetyl- | SF_Natural | 0.048783 | SF_WhiteWheat_g_wt | −0.04844 | SF_Coffee_wt | 0.03918 |
| glutamine | Yogurt_wt | | | | | |
| X - 12096 | SF_WholeWheat_g_wt | 0.066136 | SF_Baguette_wt | 0.059752 | SF_WhiteWheat_g_wt | 0.0588 |
| 1-linoleoyl- | SF_Cake_wt | −0.0673 | SF_Schnitzel_wt | −0.05655 | 3% Milk Freq | −0.04898 |
| GPA (18:2)* | | | | | | |
| X - 23654 | SF_WhiteWheat_g_wt | 0.077858 | 3-5% Natural | 0.044109 | SF_Omelette_wt | 0.034925 |
| | | | Yogurt Freq | | | |
| glycosyl-N- | SF_Milk_wt | 0.0557 | SF_Omelette_wt | −0.04808 | SF_Hummus | −0.04643 |
| stearoyl- | | | | | Salad_wt | |
| sphingosine | | | | | | |
| (d18:1/18:0) | | | | | | |
| X - 12906 | Pita Freq | −0.05466 | SF_Milk_wt | −0.04795 | Sugar | −0.04737 |
| | | | | | Sweetened | |
| | | | | | Chocolate | |
| | | | | | Milk Freq | |
| 3-sulfo-L-alanine | SF_Bread_wt | 0.061042 | Salty Snacks | 0.028345 | SF_Pretzels_wt | 0.022106 |
| | | | Freq | | | |
| X - 24498 | SF_Coffee_wt | 0.138837 | Coffee Freq | 0.055923 | SF_Rice | 0.047868 |
| | | | | | crackers_wt | |
| phosphate | SF_Pita_wt | −0.03662 | SF_WhiteWheat_g_wt | −0.03078 | Pasta or | −0.0207 |
| | | | | | Flakes Freq | |
| S-carboxymethyl- | SF_Orange_wt | −0.1069 | SF_Watermelon_wt | 0.051148 | SF_Mandarin_wt | −0.04797 |
| L-cysteine | | | | | | |
| N-oleoyltaurine | Olives Freq | 0.050767 | Lemon Freq | 0.046478 | Cauliflower or | 0.046371 |
| | | | | | Broccoli Freq | |
| cysteinylglycine | SF_Apple_wt | −0.0625 | Potatoes | 0.060617 | Shish Kebab | 0.025836 |
| | | | Boiled, | | in Pita Bread | |
| | | | Baked, | | Freq | |
| | | | Mashed, | | | |
| | | | Potatoes | | | |
| | | | Salad Freq | | | |
| X - 24699 | Falafel in Pita | 0.05733 | Beer Freq | 0.046028 | Coffee Freq | −0.04054 |
| | Bread Freq | | | | | |
| N6-succinyl- | Falafel in Pita | 0.098688 | Coffee Freq | −0.06662 | SF_Banana_wt | 0.037984 |
| adenosine | Bread Freq | | | | | |
| sphingomyelin | SF_Wholemeal | 0.023853 | SF_Banana_wt | −0.01897 | SF_Butter_wt | 0.018366 |
| (d18:0/18:0, | Light | | | | | |
| d19:0/17:0)* | Bread_wt | | | | | |
| azelate | Nuts, | 0.057165 | White or | −0.05126 | SF_Bread_wt | −0.0466 |
| (nonanedioate) | almonds, | | Brown Sugar | | | |
| | pistachios | | Freq | | | |
| | Freq | | | | | |
| X - 24813 | SF_Bread_wt | −0.04071 | SF_Cottage | 0.035005 | SF_Red | 0.033898 |
| | | | cheese_wt | | pepper_wt | |
| gamma-glutamyl-2- | Beef or | 0.048252 | Green Pepper | 0.046289 | Canned Tuna | 0.022489 |
| aminobutyrate | Chicken Soup | | Freq | | or Tuna Salad | |
| | Freq | | | | Freq | |
| 2-docosahexaenoyl- | Fish Cooked, | 0.224924 | Canned Tuna | 0.109362 | Tahini Salad | −0.06066 |
| glycerol | Baked or | | or Tuna Salad | | Freq | |
| (22:6)* | Grilled Freq | | Freq | | | |
| indoleacetate | SF_Peach_wt | 0.037143 | Carrots, Fresh | −0.03442 | Schnitzel | 0.029018 |
| | | | or Cooked, | | Turkey or | |
| | | | Carrot Juice | | Chicken Freq | |
| | | | Freq | | | |
| cis-4-decenoyl- | Artificial | −0.03686 | SF_Tahini_wt | 0.035194 | Falafel in Pita | 0.034064 |
| carnitine (C10:1) | Sweeteners | | | | Bread Freq | |
| | Freq | | | | | |
| glycerol | SF_Hummus | −0.046 | Regular Sodas | −0.04527 | Tomato Freq | −0.04509 |
| | Salad_wt | | with Sugar | | | |
| | | | Freq | | | |
| 2′-deoxyuridine | SF_Beef_wt | 0.046898 | SF_Tahini_wt | 0.044832 | SF_Bread_wt | 0.042087 |
| laurylcarnitine | Olives Freq | 0.048493 | SF_Coffee_wt | −0.03528 | SF_Yellow | 0.030481 |
| (C12) | | | | | Cheese_wt | |
| X - 12015 | Shish Kebab | 0.09333 | Green Pepper | 0.087069 | SF_Milk_wt | −0.07985 |
| | in Pita Bread | | Freq | | | |
| | Freq | | | | | |
| pro-hydroxy-pro | Orange or | 0.05136 | SF_WholeWheat_g_wt | −0.03896 | Diet Soda | −0.0292 |
| | Grapefruit | | | | Freq | |
| | Juice Freq | | | | | |
| adipate | SF_Vegetable | −0.06926 | SF_Coffee_wt | 0.064768 | SF_Potatoes_wt | −0.04173 |
| | Soup_wt | | | | | |
| malonate | SF_WhiteWheat_g_wt | −0.04652 | SF_Potatoes_wt | −0.03324 | SF_Lettuce_w t | −0.0315 |
| cystathionine | SF_Peas_wt | −0.06647 | SF_Sushi_wt | −0.03407 | SF_Cappuccino_wt | 0.026946 |
| 4-hydroxy- | SF_Tomatoes_wt | 0.043924 | SF_Wholemeal | 0.041546 | SF_WholeWheat_g_wt | 0.029764 |
| hippurate | | | Bread_wt | | | |
| eugenol sulfate | SF_Bread_wt | −0.03555 | SF_Lettuce_wt | 0.023125 | SF_Tahini_wt | 0.020809 |
| X - 24812 | Alcoholic | 0.044176 | Peach, | −0.04414 | Parsley, | 0.035591 |
| | Drinks Freq | | Nectarine, | | Celery, | |
| | | | Plum Freq | | Fennel, Dill, | |
| | | | | | Cilantro, | |
| | | | | | Green Onion | |
| | | | | | Freq | |
| 4-guanidino- | Cooked | 0.052793 | SF_Yellow | −0.04886 | SF_Wholemeal | 0.045254 |
| butanoate | Legumes Freq | | Cheese_wt | | Bread_wt | |
| X - 12718 | SF_Natural | 0.066011 | Processed | −0.04088 | SF_Coffee_wt | 0.039037 |
| | Yogurt_wt | | Meat Free | | | |
| | | | Products Freq | | | |
| X - 24519 | SF_Olives_wt | 0.060406 | Beer Freq | 0.048529 | SF_Olive | 0.039632 |
| | | | | | oil_wt | |
| 3-amino-2- | Apple Freq | 0.029171 | Peanuts Freq | 0.027707 | SF_Vegetable | 0.019886 |
| piperidone | | | | | Salad_wt | |
| N6-carbamoyl- | Falafel in Pita | 0.080872 | SF_Yellow | 0.041434 | SF_WhiteWheat_g_wt | 0.023162 |
| threonyladenosine | Bread Freq | | Cheese_wt | | | |
| 4-imidazoleacetate | SF_Wholemeal | 0.04959 | 3% Milk Freq | −0.04394 | Lemon Freq | 0.038844 |
| | Bread_wt | | | | | |
| corticosterone | SF_Rice_wt | 0.04424 | SF_Ketchup_wt | 0.035671 | SF_Hummus_wt | 0.032285 |
| DSGEGDFXAE | SF_Bread_wt | −0.03161 | Processed | −0.01426 | Beer Freq | −0.01299 |
| GGGVR* | | | Meat Free | | | |
| | | | Products Freq | | | |
| 5alpha-pregnan- | SF_Rice | −0.02416 | Pasta or | 0.022412 | Egg Recipes | 0.020874 |
| 3beta,20beta-diol | crackers_wt | | Flakes Freq | | Freq | |
| monosulfate (1) | | | | | | |
| N-acetylalliin | SF_Cucumber_wt | −0.05811 | Garlic Freq | 0.048409 | SF_Onion_wt | 0.047219 |
| salicylate | SF_Potatoes_wt | −0.03208 | Lemon Freq | 0.015662 | 5-9% Yellow | 0.014349 |
| | | | | | Cheese Freq | |
| X - 16570 | Falafel in Pita | 0.123415 | SF_Tomatoes_wt | −0.0664 | SF_Brown | −0.02451 |
| | Bread Freq | | | | Rice_wt | |
| 2-hydroxydecanoate | Nuts, | 0.073362 | 3% Milk Freq | −0.043 | Cooked | 0.037346 |
| | almonds, | | | | Legumes Freq | |
| | pistachios | | | | | |
| | Freq | | | | | |
| isovalerylglycine | SF_Coffee_wt | 0.05993 | Egg, Hard | 0.046294 | Artificial | 0.044144 |
| | | | Boiled or Soft | | Sweeteners | |
| | | | Freq | | Freq | |
| sphingomyelin | Coffee Freq | 0.058736 | SF_Wholemeal | 0.039402 | SF_Dark | 0.026623 |
| (d18:0/20:0, | | | Light | | Chocolate_wt | |
| d16:0/22:0)* | | | Bread_wt | | | |
| alliin | Garlic Freq | 0.07817 | SF_Onion_wt | 0.066297 | Diet Yogurt | −0.0454 |
| | | | | | Freq | |
| docosapentaenoate | SF_Vegetable | −0.04399 | SF_Cookies_wt | −0.03842 | SF_Egg_wt | 0.038342 |
| (n6 DPA; 22:5n6) | Salad_wt | | | | | |
| dodecadienoate | Nuts, | 0.055041 | SF_Tahini_wt | 0.05432 | Tahini Salad | 0.033912 |
| (12:2)* | almonds, | | | | Freq | |
| | pistachios | | | | | |
| | Freq | | | | | |
| 2-methoxyresorcinol | SF_Coffee_wt | 0.062075 | SF_WholeWheat_g_wt | 0.041962 | Coffee Freq | 0.03172 |
| sulfate | | | | | | |
| biliverdin | Alcoholic | 0.044794 | Brussels | −0.03934 | Beer Freq | 0.037637 |
| | Drinks Freq | | Sprouts, | | | |
| | | | Green or Red | | | |
| | | | Cabbage Freq | | | |
| oleate/vaccenate | Regular Sodas | −0.02705 | SF_Noodles_wt | −0.02665 | Olives Freq | 0.025082 |
| (18:1) | with Sugar | | | | | |
| | Freq | | | | | |
| 1,2-dipalmitoyl- | SF_Tahini_wt | −0.05504 | Peach, | 0.021556 | Artificial | 0.017402 |
| GPC | | | Nectarine, | | Sweeteners | |
| (16:0/16:0) | | | Plum Freq | | Freq | |
| X - 23787 | SF_Peas_wt | 0.041056 | SF_Coffee_wt | −0.03616 | SF_Dark | 0.033285 |
| | | | | | Chocolate_wt | |
| 5alpha-androstan- | Beer Freq | 0.071782 | SF_Ice | 0.026726 | SF_Brown | −0.02326 |
| 3beta,17alpha- | | | cream_wt | | Rice_wt | |
| diol disulfate | | | | | | |
| N-acetylleucine | Processed | −0.04816 | SF_Carrots_wt | −0.04546 | Banana Freq | −0.04531 |
| | Meat Free | | | | | |
| | Products Freq | | | | | |
| X - 16397 | SF_Tahini_wt | −0.11933 | SF_WhiteWheat_g_wt | −0.03252 | Granola or | −0.02613 |
| | | | | | Bernflaks | |
| | | | | | Freq | |
| hypoxanthine | >=16% Yellow | 0.030625 | SF_Bread_wt | 0.019789 | SF_Hummus | −0.01735 |
| | Cheese Freq | | | | Salad_wt | |
| guanidinosuccinate | SF_Cottage | 0.027991 | Beef or | 0.027637 | SF_Vegetable | 0.024513 |
| | cheese_wt | | Chicken Soup | | Salad_wt | |
| | | | Freq | | | |
| oleoylcholine | SF_Bread_wt | 0.036108 | Sugar | 0.030004 | Apricot Fresh | 0.02974 |
| | | | Sweetened | | or Dry, or | |
| | | | Chocolate | | Loquat Freq | |
| | | | Milk Freq | | | |
| X - 11530 | Ordinary | 0.038735 | SF_Beer_wt | 0.03738 | Red Pepper | −0.01961 |
| | Bread or | | | | Freq | |
| | Challah Freq | | | | | |
| sphingomyelin | Beer Freq | −0.05029 | SF_Hummus | −0.03659 | SF_WhiteWheat_g_wt | −0.03544 |
| (d18:2/16:0, | | | Salad_wt | | | |
| d18:1/16:1)* | | | | | | |
| 1-stearoyl-2- | Beef, Veal, | −0.05587 | SF_Rice | 0.038722 | SF_Coffee_wt | 0.027619 |
| linoleoyl-GPE | Lamb, Pork, | | crackers_wt | | | |
| (18:0/18:2)* | Steak, Golash | | | | | |
| | Freq | | | | | |
| phenyllactate | SF_White | 0.027312 | Beer Freq | 0.024407 | Kiwi or | −0.01453 |
| (PLA) | beans_wt | | | | Strawberries | |
| | | | | | Freq | |
| methylsuccinate | Coffee Freq | 0.089331 | SF_Coffee_wt | 0.082275 | SF_Tzfatit | 0.030701 |
| | | | | | Cheese_wt | |
| X - 18887 | Artificial | −0.04104 | Tahini Salad | 0.03989 | SF_Potatoes_wt | 0.024166 |
| | Sweeteners | | Freq | | | |
| | Freq | | | | | |
| X - 21286 | SF_Natural | 0.041114 | Parsley, | −0.03748 | SF_White | 0.035784 |
| | Yogurt_wt | | Celery, | | Cheese_wt | |
| | | | Fennel, Dill, | | | |
| | | | Cilantro, | | | |
| | | | Green Onion | | | |
| | | | Freq | | | |
| gamma-glutamyl- | SF_Tomatoes_wt | −0.05988 | SF_Vegetable | 0.053782 | Coffee Freq | −0.04005 |
| citrulline* | | | Salad_wt | | | |
| glycodeoxy- | SF_Tahini_wt | −0.04515 | Fresh | −0.03848 | Green Tea | −0.03742 |
| cholate sulfate | | | Vegetable | | Freq | |
| | | | Salad Without | | | |
| | | | Dressing or | | | |
| | | | Oil Freq | | | |
| 3-hydroxylaurate | SF_Yellow | 0.05357 | Olives Freq | 0.030034 | SF_WhiteWheat_g_wt | −0.01863 |
| | Cheese_wt | | | | | |
| sulfate of | SF_Pickled | 0.083888 | Parsley, | 0.059971 | SF_Milk_wt | −0.04173 |
| piperine | cucumber_wt | | Celery, | | | |
| metabolite | | | Fennel, Dill, | | | |
| C16H19NO3 | | | Cilantro, | | | |
| (2)* | | | Green Onion | | | |
| | | | Freq | | | |
| 1-carboxyethyl- | 5-9% Yellow | 0.041844 | Wholemeal or | −0.04121 | SF_Fried | −0.03209 |
| leucine | Cheese Freq | | Rye Bread | | eggplant_wt | |
| | | | Freq | | | |
| sebacate | Sausages | 0.037883 | SF_Yellow | 0.036507 | Carrots, Fresh | −0.03597 |
| (decanedioate) | such as | | Cheese_wt | | or Cooked, | |
| | Salami Freq | | | | Carrot Juice | |
| | | | | | Freq | |
| N-acetylneuraminate | SF_Hummus | −0.00926 | SF_Bread_wt | 0.009013 | Olives Freq | 0.003914 |
| | Salad_wt | | | | | |
| N-formylanthranilic | Processed | −0.09354 | SF_Natural | 0.066743 | Onion Freq | −0.04453 |
| acid | Meat Free | | Yogurt_wt | | | |
| | Products Freq | | | | | |
| picolinate | SF_Coffee_wt | 0.071362 | Coffee Freq | 0.059671 | SF_WholeWheat_g_wt | −0.04863 |
| 4-hydroxybenzoate | SF_Cranberries_wt | −0.03406 | SF_Tea_wt | 0.029731 | SF_Yellow | 0.027116 |
| | | | | | Cheese_wt | |
| 2- hydroxybehenate | SF_WhiteWheat_g_wt | −0.04678 | SF_Noodles_wt | −0.03738 | Tomato Freq | −0.03107 |
| 5-dodecenoate | Regular Sodas | −0.06199 | SF_Yellow | 0.035471 | SF_Milk_wt | 0.021621 |
| (12:1n7) | with Sugar | | Cheese_wt | | | |
| | Freq | | | | | |
| X - 12831 | SF_Wholemeal | 0.023572 | SF_Soymilk_wt | 0.022402 | SF_WhiteWheat_g_wt | 0.017332 |
| | Bread_wt | | | | | |
| glycerol 3-phosphate | SF_Pita_wt | −0.0328 | SF_Bread_wt | 0.016143 | SF_Cottage | −0.01372 |
| | | | | | cheese_wt | |
| N-palmitoyltaurine | SF_Olives_wt | 0.032981 | SF_Butter_wt | 0.028811 | Butter Freq | 0.027231 |
| octadecadiene | SF_Cottage | 0.053773 | SF_Rice_wt | 0.051693 | Red Pepper | 0.042027 |
| dioate (C18:2- | cheese_wt | | | | Freq | |
| DC)* | | | | | | |
| 1-stearoyl-GPE | SF_Coffee_wt | 0.027216 | Salty Cheese, | 0.026333 | SF_Diet | −0.02553 |
| (18:0) | | | Tzfatit, | | Coke_wt | |
| | | | Bulgarian, | | | |
| | | | Brinza, Thick | | | |
| | | | Slice Freq | | | |
| bilirubin (E, E)* | Alcoholic | 0.050502 | Beer Freq | 0.032377 | SF_Rice_wt | 0.026276 |
| | Drinks Freq | | | | | |
| N-acetylthreonine | SF_WhiteWheat_g_wt | 0.052422 | Orange or | 0.042283 | 3% Milk Freq | −0.02683 |
| | | | Grapefruit | | | |
| | | | Freq | | | |
| homoarginine | SF_Potatoes_wt | 0.070383 | SF_Water_wt | −0.04607 | SF_Vegetable | 0.033306 |
| | | | | | Salad_wt | |
| tetradecanedioate | Tahini Salad | −0.11566 | SF_Tahini_wt | −0.08221 | Fish Cooked, | 0.048859 |
| | Freq | | | | Baked or | |
| | | | | | Grilled Freq | |
| 12-HETE | SF_Bread_wt | 0.026539 | SF_Cottage | −0.02202 | SF_Cake_wt | −0.01692 |
| | | | cheese_wt | | | |
| X - 11843 | Parsley, | −0.02759 | Oil as an | −0.02729 | SF_Soda | −0.02582 |
| | Celery, | | addition for | | water_wt | |
| | Fennel, Dill, | | Salads or | | | |
| | Cilantro, | | Stews Freq | | | |
| | Green Onion | | | | | |
| | Freq | | | | | |
| X - 22771 | Carrots, Fresh | 0.063908 | SF_Vegetable | 0.042727 | SF_Cranberries_wt | 0.042558 |
| | or Cooked, | | Salad_wt | | | |
| | Carrot Juice | | | | | |
| | Freq | | | | | |
| 2,3-dihydroxy- | Cooked | −0.05082 | SF_Vegetable | 0.044911 | SF_Sugar Free | −0.02946 |
| 5-methylthio- | Cereal such as | | Salad_wt | | Gum_wt | |
| 4-pentenoate | Oatmeal | | | | | |
| (DMTPA)* | Porridge Freq | | | | | |
| myristoleoyl- | Olives Freq | 0.064021 | SF_Coffee_wt | −0.04737 | 1% Milk Freq | −0.02303 |
| carnitine | | | | | | |
| (C14:1)* | | | | | | |
| orotidine | Falafel in Pita | 0.044403 | Pastrami or | 0.033915 | SF_Tomatoes_wt | −0.0332 |
| | Bread Freq | | Smoked Turkey | | | |
| | | | Breast Freq | | | |
| X - 18345 | Wholemeal or | −0.08058 | SF_Water_wt | 0.037649 | Ordinary | −0.03263 |
| | Rye Bread | | | | Bread or | |
| | Freq | | | | Challah Freq | |
| N-palmitoyl- | SF_Hummus | −0.046 | SF_Potatoes_wt | −0.02982 | SF_WhiteWheat_g_wt | −0.0291 |
| sphingadienine | Salad_wt | | | | | |
| (d18:2/16:0)* | | | | | | |
| glutarate | SF_Chicken | 0.039986 | Beef, Veal, | 0.031258 | Coated or | 0.02802 |
| (pentanedioate) | breast_wt | | Lamb, Pork, | | Stuffed | |
| | | | Steak, Golash | | Cookies, | |
| | | | Freq | | Waffles or | |
| | | | | | Biscuits Freq | |
| ornithine | SF_Vegetable | 0.068277 | White or | −0.03168 | Apple Freq | 0.023569 |
| | Salad_wt | | Brown Sugar | | | |
| | | | Freq | | | |
| 1-palmitoyl-2- | SF_Rice | 0.051376 | Beef, Veal, | −0.04192 | Turkey | −0.02903 |
| linoleoyl-GPE | crackers_wt | | Lamb, Pork, | | Meatballs, | |
| (16:0/18:2) | | | Steak, Golash | | Beef, Chicken | |
| | | | Freq | | Freq | |
| X - 24512 | SF_Coffee_wt | 0.042437 | SF_White | 0.034453 | Mandarin or | 0.032431 |
| | | | beans_wt | | Clementine | |
| | | | | | Freq | |
| dopamine 3- | SF_Banana_wt | 0.026065 | SF_Wholemeal | 0.024085 | SF_Tomatoes_wt | 0.02404 |
| O-sulfate | | | Bread_wt | | | |
| isovalerate | SF_Carrots_wt | −0.0503 | SF_Schnitzel_wt | 0.020979 | White or | −0.01881 |
| | | | | | Brown Sugar | |
| | | | | | Freq | |
| 1-palmitoyl- | SF_Onion_wt | −0.02934 | Peanuts Freq | −0.02909 | SF_Carrots_wt | −0.02579 |
| GPG (16:0)* | | | | | | |
| 14-HDoHE/17- | SF_Bread_wt | 0.022108 | Fish Cooked, | 0.007253 | SF_Mandarin_wt | 0.00551 |
| HDoHE | | | Baked or | | | |
| | | | Grilled Freq | | | |
| 1-palmitoyl- | SF_Tahini_wt | −0.02778 | SF_Ice | 0.021235 | SF_Almonds_wt | −0.02079 |
| GPI (16:0) | | | cream_wt | | | |
| trans- | SF_Pizza_wt | 0.019236 | Fresh | 0.018702 | SF_Ice | 0.01836 |
| urocanate | | | Vegetable | | cream_wt | |
| | | | Salad Without | | | |
| | | | Dressing or | | | |
| | | | Oil Freq | | | |
| X - 21842 | SF_Tahini_wt | 0.028064 | Egg, Hard | 0.027219 | SF_Hummus_wt | 0.020722 |
| | | | Boiled or Soft | | | |
| | | | Freq | | | |
| xanthurenate | SF_Omelette_wt | 0.112054 | SF_Natural | 0.093045 | SF_Sugar_wt | −0.07047 |
| | | | Yogurt_wt | | | |
| N-acetylglutamate | SF_Couscous_wt | −0.02509 | SF_Coffee_wt | 0.020418 | SF_Tomatoes_wt | −0.01222 |
| phospho- | SF_Cottage | −0.03097 | Schnitzel | −0.01953 | SF_Water_wt | 0.019053 |
| ethanolamine | cheese_wt | | Turkey or | | | |
| | | | Chicken Freq | | | |
| 1-(1-enyl- | Hummus | −0.06382 | Beef or | 0.043133 | Orange or | 0.042737 |
| palmitoyl)-2- | Salad Freq | | Chicken Soup | | Grapefruit | |
| palmitoyl-GPC | | | Freq | | Freq | |
| (P-16:0/16:0)* | | | | | | |
| hexadecene- | SF_Tahini_wt | −0.05817 | Carrots, Fresh | −0.03807 | Tahini Salad | −0.03629 |
| dioate (C16:1- | | | or Cooked, | | Freq | |
| DC)* | | | Carrot Juice | | | |
| | | | Freq | | | |
| X - 12822 | SF_Coffee_wt | −0.03808 | SF_Apple_wt | −0.03269 | Wholemeal or | −0.02946 |
| | | | | | Rye Bread | |
| | | | | | Freq | |
| X - 21607 | Tahini Salad | 0.089491 | SF_Tahini_wt | 0.074356 | Nuts, | 0.054303 |
| | Freq | | | | almonds, | |
| | | | | | pistachios | |
| | | | | | Freq | |
| epiandrosterone | Beer Freq | 0.086832 | SF_Pita_wt | 0.025957 | Salty Cheese, | −0.0197 |
| sulfate | | | | | Tzfatit, | |
| | | | | | Bulgarian, | |
| | | | | | Brinza, Thin | |
| | | | | | Slice Freq | |
| 2-keto-3-deoxy- | SF_Almonds_wt | 0.062034 | Falafel in Pita | 0.052251 | SF_Cappuccino_wt | −0.02988 |
| gluconate | | | Bread Freq | | | |
| hydroxy- | SF_Sugar Free | −0.03736 | SF_Carrots_wt | −0.03496 | SF_Yellow | 0.022331 |
| asparagine** | Gum_wt | | | | Cheese_wt | |
| uridine | SF_Bread_wt | 0.03592 | Onion Freq | 0.009274 | SF_Rice_wt | 0.00823 |
| 5-(galactosyl- | SF_Sugar Free | −0.08282 | Pastrami or | 0.026033 | SF_Carrots_wt | −0.01667 |
| hydroxy)-L-lysine | Gum_wt | | Smoked Turkey | | | |
| | | | Breast Freq | | | |
| ceramide | Cooked | −0.07797 | SF_Jam_wt | 0.052299 | SF_Natural | 0.04184 |
| (d16:1/24:1, | Legumes Freq | | | | Yogurt_wt | |
| d18:1/22:1)* | | | | | | |
| glycosyl | SF_Milk_wt | 0.073216 | Butter Freq | 0.050512 | Fresh | 0.037208 |
| ceramide | | | | | Vegetable | |
| (d18:1/20:0, | | | | | Salad With | |
| d16:1/22:0)* | | | | | Dressing or | |
| | | | | | Oil Freq | |
| 1-stearoyl-2- | SF_WhiteWheat_g_wt | −0.07191 | SF_Bread_wt | −0.02015 | SF_Mayonnaise_wt | −0.01374 |
| oleoyl-GPI | | | | | | |
| (18:0/18:1)* | | | | | | |
| X - 12013 | SF_Schnitzel_wt | 0.037638 | Oil as an | −0.03406 | SF_Water_wt | 0.033615 |
| | | | addition for | | | |
| | | | Salads or | | | |
| | | | Stews Freq | | | |
| 3-hydroxydecanoate | Butter Freq | 0.063398 | SF_Yellow | 0.03525 | Nuts, | 0.034922 |
| | | | Cheese_wt | | almonds, | |
| | | | | | pistachios | |
| | | | | | Freq | |
| anthranilate | SF_Natural | 0.064806 | Parsley, | −0.04542 | 5-9% Yellow | 0.038353 |
| | Yogurt_wt | | Celery, | | Cheese Freq | |
| | | | Fennel, Dill, | | | |
| | | | Cilantro, | | | |
| | | | Green Onion | | | |
| | | | Freq | | | |
| 5-methyluridine | Chicken or | −0.03894 | Olives Freq | 0.024302 | SF_Hummus | 0.022257 |
| (ribothymidine) | Turkey | | | | Salad_wt | |
| | Without Skin | | | | | |
| | Freq | | | | | |
| 5-bromotryptophan | SF_Coffee_wt | −0.04578 | Fries Freq | 0.030686 | SF_Diet | −0.02692 |
| | | | | | Coke_wt | |
| 1-(1-enyl- | Alcoholic | 0.09033 | SF_Potatoes_wt | −0.035 | 5-9% Yellow | −0.03055 |
| palmitoyl)-2- | Drinks Freq | | | | Cheese Freq | |
| linoleoyl-GPC | | | | | | |
| (P-16:0/18:2)* | | | | | | |
| 3-hydroxybutyryl- | Falafel in Pita | 0.054557 | Peach, | −0.04312 | Herbal Tea | −0.0317 |
| carnitine (2) | Bread Freq | | Nectarine, | | Freq | |
| | | | Plum Freq | | | |
| pregnanolone/ | SF_Pizza_wt | 0.038135 | Peanuts Freq | −0.03626 | Tahini Salad | −0.03566 |
| allopregnanolone | | | | | Freq | |
| sulfate | | | | | | |
| X - 24728 | Falafel in Pita | 0.110368 | Chicken or | −0.06747 | SF_Olives_wt | 0.055402 |
| | Bread Freq | | Turkey | | | |
| | | | Without Skin | | | |
| | | | Freq | | | |
| 1-oleoyl-GPI | Sweet Potato | 0.032519 | SF_Cooked | −0.03121 | SF_Hummus | −0.03033 |
| (18:1)* | Freq | | mushrooms_wt | | Salad_wt | |
| glycine | SF_Cold | −0.02109 | SF_Potatoes_wt | −0.01852 | Cooked | 0.017126 |
| | cut_wt | | | | Legumes Freq | |
| dihomo- | Regular Sodas | −0.03101 | Simple | −0.02365 | 5-9% White | −0.01606 |
| linoleate | with Sugar | | Cookies or | | Cheese, | |
| (20:2n6) | Freq | | Biscuits Freq | | Cottage Freq | |
| 2-linoleoyl- | SF_WhiteWheat_g_wt | 0.028344 | Coffee Freq | −0.01592 | SF_Egg_wt | −0.01307 |
| glycerol (18:2) | | | | | | |
| citrulline | SF_Vegetable | 0.057484 | Apple Freq | 0.041661 | Garlic Freq | −0.02262 |
| | Salad_wt | | | | | |
| lactosyl-N- | SF_WholeWheat_g_wt | 0.06849 | Light Bread | −0.03841 | SF_Raisins_wt | 0.03672 |
| behenoyl- | | | Freq | | | |
| sphingosine | | | | | | |
| (d18:1/22:0)* | | | | | | |
| 1-palmitoleoyl- | Beef, Veal, | −0.05869 | SF_Olives_wt | −0.04186 | Egg Recipes | −0.03806 |
| 2-linolenoyl- | Lamb, Pork, | | | | Freq | |
| GPC | Steak, Golash | | | | | |
| (16:1/18:3)* | Freq | | | | | |
| bilirubin (Z, Z) | Ordinary | 0.060268 | SF_Rice_wt | 0.03322 | Beer Freq | 0.013835 |
| | Bread or | | | | | |
| | Challah Freq | | | | | |
| 4-acetamido- | SF_Cucumber_wt | 0.028783 | SF_Red | 0.025129 | Tahini Salad | 0.022158 |
| benzoate | | | pepper_wt | | Freq | |
| docosadienoate | Regular Sodas | −0.03938 | Simple | −0.02318 | Nuts, | 0.018016 |
| (22:2n6) | with Sugar | | Cookies or | | almonds, | |
| | Freq | | Biscuits Freq | | pistachios | |
| | | | | | Freq | |
| vanillactate | 3% Milk Freq | −0.08107 | SF_Coffee_wt | 0.049414 | SF_Olives_wt | 0.042424 |
| taurodeoxy- | SF_Tahini_wt | −0.07236 | SF_Hummus | −0.05778 | Falafel in Pita | −0.05085 |
| cholic acid 3- | | | Salad_wt | | Bread Freq | |
| sulfate | | | | | | |
| X - 12126 | SF_Natural | 0.108942 | SF_Coffee_wt | 0.087501 | Mandarin or | 0.050183 |
| | Yogurt_wt | | | | Clementine | |
| | | | | | Freq | |
| stearate (18:0) | Simple | −0.01505 | Wholemeal or | −0.01393 | Juice Freq | −0.00952 |
| | Cookies or | | Rye Bread | | | |
| | Biscuits Freq | | Freq | | | |
| indolelactate | Sausages Freq | 0.033497 | Beer Freq | 0.032095 | SF_Omelette_wt | 0.01488 |
| X - 13684 | SF_Coffee_wt | −0.07093 | SF_Apple_wt | −0.04044 | SF_WhiteWheat_g_wt | 0.037778 |
| sulfate of | Parsley, | 0.064539 | SF_Pickled | 0.055137 | Beer Freq | 0.035846 |
| piperine | Celery, | | cucumber_wt | | | |
| metabolite | Fennel, Dill, | | | | | |
| C16H19NO3 | Cilantro, | | | | | |
| (3)* | Green Onion | | | | | |
| | Freq | | | | | |
| X - 24309 | Butter Freq | 0.081048 | SF_Butter_wt | 0.037678 | Fresh | −0.03729 |
| | | | | | Vegetable | |
| | | | | | Salad Without | |
| | | | | | Dressing or | |
| | | | | | Oil Freq | |
| 1-(1-enyl- | SF_Hummus | −0.05849 | Orange or | 0.041467 | Hummus | −0.0208 |
| palmitoyl)-2- | Salad_wt | | Grapefruit | | Salad Freq | |
| palmitoleoyl-GPC | | | Freq | | | |
| (P-16:0/16:1)* | | | | | | |
| N-acetyl-S- | SF_Pizza_wt | 0.04455 | SF_Pita_wt | 0.024971 | SF_Tabbouleh | 0.01793 |
| allyl-L-cysteine | | | | | Salad_wt | |
| 2-oxoarginine* | SF_Apple_wt | 0.032075 | Baguette Freq | −0.02751 | Cooked | 0.025905 |
| | | | | | Legumes Freq | |
| dihomo- | Egg, Hard | −0.02306 | 0.5-3% White | 0.02025 | Light Bread | 0.017183 |
| linolenate | Boiled or Soft | | Cheese, | | Freq | |
| (20:3n3 or n6) | Freq | | Cottage Freq | | | |
| glycochenode | SF_Water_wt | −0.04156 | SF_Rice_wt | 0.033314 | 3% Milk Freq | −0.03251 |
| oxycholate | | | | | | |
| glucuronide | | | | | | |
| (1) | | | | | | |
| N,N-dimethyl-5- | >=16% Yellow | −0.0339 | Fries Freq | −0.0291 | SF_Omelette_wt | −0.01767 |
| aminovalerate | Cheese Freq | | | | | |
| taurocholate | SF_Cereals_wt | 0.034716 | SF_Peas_wt | −0.03336 | SF_Milk_wt | −0.03277 |
| 2-hydroxyadipate | SF_Cappuccino_wt | 0.089041 | White or | −0.03949 | Coffee Freq | 0.032804 |
| | | | Brown Sugar | | | |
| | | | Freq | | | |
| mannose | Simple | −0.0374 | Onion Freq | 0.022124 | SF_Couscous_wt | −0.01827 |
| | Cookies or | | | | | |
| | Biscuits Freq | | | | | |
| X - 19561 | 5-9% Yellow | 0.068141 | Green Tea | −0.04406 | SF_White | 0.039535 |
| | Cheese Freq | | Freq | | Cheese_wt | |
| N-acetylalanine | SF_Yellow | 0.039334 | SF_Cooked | −0.01135 | SF_Tomatoes_wt | −0.01106 |
| | Cheese_wt | | Sweet | | | |
| | | | potato_wt | | | |
| phenylpyruvate | Egg Recipes | 0.050362 | SF_Wholemeal | 0.009452 | 5-9% Yellow | 0.006684 |
| | Freq | | Bread_wt | | Cheese Freq | |
| stearoylcholine* | SF_Bread_wt | 0.04418 | Apricot Fresh | 0.035031 | SF_Salty | 0.0263 |
| | | | or Dry, or | | Cheese_wt | |
| | | | Loquat Freq | | | |
| palmitoleoyl- | Olives Freq | 0.090714 | SF_Coffee_wt | −0.03917 | Simple | −0.03007 |
| carnitine | | | | | Cookies or | |
| (C16:1)* | | | | | Biscuits Freq | |
| 2-palmitoleoyl- | SF_Tahini_wt | −0.0444 | SF_Water_wt | −0.04329 | SF_White | 0.043042 |
| GPC (16:1)* | | | | | Cheese_wt | |
| phenol sulfate | Artificial | 0.066369 | Cake, Torte | −0.062 | SF_Cookies_wt | −0.03456 |
| | Sweeteners | | Cakes, | | | |
| | Freq | | Chocolate | | | |
| | | | Cake Freq | | | |
| X - 23739 | 3% Milk Freq | −0.02238 | 5-9% White | −0.00982 | SF_Yellow | −0.009 |
| | | | Cheese, | | Cheese_wt | |
| | | | Cottage Freq | | | |
| 2-stearoyl-GPE | Pita Freq | −0.0259 | SF_Coffee_wt | 0.024649 | Salty Cheese, | 0.020803 |
| (18:0)* | | | | | Tzfatit, | |
| | | | | | Bulgarian, | |
| | | | | | Brinza, Thick | |
| | | | | | Slice Freq | |
| glycerate | Red Pepper | 0.034476 | Green Pepper | 0.031018 | SF_WhiteWheat_g_wt | −0.02396 |
| | Freq | | Freq | | | |
| X - 12100 | SF_Rice_wt | 0.026018 | SF_Tomatoes_wt | 0.010053 | 3-5% Natural | 0.00783 |
| | | | | | Yogurt Freq | |
| 5alpha-pregnan- | Mandarin or | −0.03349 | Egg Recipes | 0.027443 | SF_Water_wt | 0.024725 |
| 3beta,20alpha- | Clementine | | Freq | | | |
| diol disulfate | Freq | | | | | |
| phenylalanyl- | SF_Cake_wt | −0.06233 | SF_Hummus | −0.04755 | SF_Wholemeal | −0.03926 |
| glycine | | | Salad_wt | | Bread_wt | |
| heptanoate | SF_Tomatoes_wt | −0.04937 | SF_Tahini_wt | 0.043777 | Sugar | −0.0332 |
| (7:0) | | | | | Sweetened | |
| | | | | | Chocolate | |
| | | | | | Milk Freq | |
| 4-acetamido- | Apple Freq | 0.042723 | SF_Hummus | −0.03426 | SF_Sweet | −0.02447 |
| butanoate | | | Salad_wt | | potato_wt | |
| thyroxine | SF_Vegetable | −0.05101 | SF_Banana_wt | −0.04419 | SF_Hummus | −0.04165 |
| | Salad_wt | | | | Salad_wt | |
| 1-oleoyl-GPC | Light Bread | −0.02775 | Chicken or | −0.02671 | 5-9% Yellow | −0.01823 |
| (18:1) | Freq | | Turkey | | Cheese Freq | |
| | | | Without Skin | | | |
| | | | Freq | | | |
| linoleate | Nuts, | 0.054444 | Chicken or | −0.03123 | Regular Sodas | −0.02419 |
| (18:2n6) | almonds, | | Turkey | | with Sugar | |
| | pistachios | | Without Skin | | Freq | |
| | Freq | | Freq | | | |
| galactonate | SF_Coffee_wt | 0.085652 | 3% Milk Freq | 0.054521 | SF_Bread_wt | −0.0472 |
| octanoyl- | Olives Freq | 0.046119 | SF_Watermelon_wt | −0.0244 | 1% Milk Freq | −0.02394 |
| carnitine (C8) | | | | | | |
| piperine | Parsley, | 0.050012 | SF_Pickled | 0.037475 | Onion Freq | 0.023818 |
| | Celery, | | cucumber_wt | | | |
| | Fennel, Dill, | | | | | |
| | Cilantro, | | | | | |
| | Green Onion | | | | | |
| | Freq | | | | | |
| N-acetylproline | SF_Olive | 0.062883 | SF_Bread_wt | −0.0477 | 0-1.5% | −0.03521 |
| | oil_wt | | | | Lebbem, | |
| | | | | | Eshel Freq | |
| X - 12216 | SF_Coffee_wt | 0.052934 | SF_Natural | 0.044457 | 5-9% White | 0.039397 |
| | | | Yogurt_wt | | Cheese, | |
| | | | | | Cottage Freq | |
| 2-hydroxyglutarate | SF_Coffee_wt | 0.044363 | SF_Wine_wt | 0.039629 | SF_Cooked | −0.02253 |
| | | | | | beets_wt | |
| choline | SF_Bread_wt | 0.015375 | Orange or | −0.00538 | SF_Chocolate | 0.004498 |
| | | | Grapefruit | | _wt | |
| | | | Freq | | | |
| 2,2′-Methylene- | SF_Yellow | −0.10353 | SF_Tea_wt | −0.07441 | SF_Rice_wt | −0.06332 |
| bis(6-tert- | Cheese_wt | | | | | |
| butyl-p-cresol) | | | | | | |
| 5,6-dihydrouridine | Chicken or | −0.05223 | Falafel in Pita | 0.032329 | Egg, Hard | −0.03073 |
| | Turkey | | Bread Freq | | Boiled or Soft | |
| | Without Skin | | | | Freq | |
| | Freq | | | | | |
| cis-4-decenoate | SF_Tahini_wt | 0.040137 | Nuts, | 0.03645 | Tahini Salad | 0.019534 |
| (10:1n6)* | | | almonds, | | Freq | |
| | | | pistachios | | | |
| | | | Freq | | | |

| | Top | Directional | Top | Directional | Diet | |
|---|---|---|---|---|---|---|
| | predictor | SHAP value | predictor | SHAP value | Pearson | Diet |
| BIOCHEMICAL | #4 | #4 | #5 | #5 | R | p-value |
| 1-methylxanthine | 3% Milk Freq | 0.056886 | Alcoholic | 0.044545 | 0.739589 | 2.31E−83 |
| | | | Drinks Freq | | | |
| 3-carboxy-4- | Simple | −0.06085 | SF_Dark | 0.053774 | 0.736143 | 3.25E−82 |
| methyl-5- | Cookies or | | Chocolate_wt | | | |
| propyl-2- | Biscuits Freq | | | | | |
| furanpropanoate | | | | | | |
| (CMPF) | | | | | | |
| hydroxy-CMPF* | Tahini Salad | −0.05219 | SF_Tahini_wt | −0.04784 | 0.71885 | 1.03E−76 |
| | Freq | | | | | |
| quinate | Apricot Fresh | 0.039115 | SF_Rice | 0.032074 | 0.716823 | 4.29E−76 |
| | or Dry, or | | crackers_wt | | | |
| | Loquat Freq | | | | | |
| X - 21442 | SF_Wine_wt | 0.064685 | Beer Freq | 0.036059 | 0.715901 | 8.16E−76 |
| 1-methylurate | 3% Milk Freq | 0.035725 | SF_Tomatoes_wt | −0.03072 | 0.695027 | 8.90E−70 |
| 1,3-dimethylurate | SF_Onion_wt | −0.06041 | 3% Milk Freq | 0.058214 | 0.694824 | 1.01E−69 |
| 1,3,7-trimethylurate | SF_Wine_wt | 0.071726 | SF_Fried | −0.04764 | 0.684333 | 7.01E−67 |
| | | | onions_wt | | | |
| X - 24811 | SF_Rice | 0.08607 | SF_Carrots_wt | −0.04638 | 0.684028 | 8.45E−67 |
| | crackers_wt | | | | | |
| theophylline | SF_Wine_wt | 0.073244 | Regular Sodas | −0.05714 | 0.681862 | 3.15E−66 |
| | | | with Sugar | | | |
| | | | Freq | | | |
| 5-acetylamino- | SF_Cappuccino_wt | 0.053248 | >=16% Yellow | 0.036118 | 0.680865 | 5.73E−66 |
| 6-amino-3- | | | Cheese Freq | | | |
| methyluracil | | | | | | |
| 1,7-dimethylurate | Cooked | −0.04896 | SF_Wine_wt | 0.048168 | 0.675881 | 1.12E−64 |
| | Legumes Freq | | | | | |
| caffeine | SF_Cappuccino_wt | 0.035141 | Cooked | −0.03506 | 0.666618 | 2.39E−62 |
| | | | Legumes Freq | | | |
| paraxanthine | SF_Noodles_wt | −0.0856 | SF_Wine_wt | 0.075814 | 0.647124 | 1.05E−57 |
| X - 23655 | SF_Natural | 0.057576 | Mixed | 0.041744 | 0.628911 | 1.15E−53 |
| | Yogurt_wt | | Chicken or | | | |
| | | | Turkey Dishes | | | |
| | | | Freq | | | |
| X - 13835 | Chicken or | 0.103375 | Processed | −0.09108 | 0.625356 | 6.56E−53 |
| | Turkey | | Meat Free | | | |
| | Without Skin | | Products Freq | | | |
| | Freq | | | | | |
| saccharin | Diet Soda | 0.01876 | SF_Beer_wt | −0.01812 | 0.613653 | 1.75E−50 |
| | Freq | | | | | |
| 3-methyl catechol | Butter Freq | 0.050491 | SF_Fried | −0.03913 | 0.611563 | 4.62E−50 |
| sulfate (1) | | | onions_wt | | | |
| 3-hydroxypyridine | SF_Cappuccino_wt | 0.034677 | SF_Natural | 0.03045 | 0.610799 | 6.58E−50 |
| sulfate | | | Yogurt_wt | | | |
| X - 23652 | Chicken or | 0.073229 | Processed | −0.06307 | 0.602815 | 2.51E−48 |
| | Turkey | | Meat Free | | | |
| | Without Skin | | Products Freq | | | |
| | Freq | | | | | |
| trigonelline (N′- | SF_Rice | 0.053792 | Regular Sodas | −0.04124 | 0.59323 | 1.74E−46 |
| methylnicotinate) | crackers_wt | | with Sugar | | | |
| | | | Freq | | | |
| X - 11315 | SF_Rice | 0.061804 | White or | −0.05896 | 0.590039 | 6.89E−46 |
| | crackers_wt | | Brown Sugar | | | |
| | | | Freq | | | |
| 1-methyl-5- | Chicken or | 0.083141 | Processed | −0.0737 | 0.587075 | 2.45E−45 |
| imidazoleacetate | Turkey | | Meat Free | | | |
| | Without Skin | | Products Freq | | | |
| | Freq | | | | | |
| 1-(1-enyl-palmitoyl)- | Egg, Hard | 0.087188 | Cooked | −0.07198 | 0.582208 | 1.91E−44 |
| 2-arachidonoyl-GPE | Boiled or Soft | | Legumes Freq | | | |
| (P-16:0/20:4)* | Freq | | | | | |
| X - 11858 | Hummus | 0.064557 | SF_Vegetable | 0.042072 | 0.577807 | 1.18E−43 |
| | Salad Freq | | Salad_wt | | | |
| 1-(1-enyl-stearoyl)- | Beef or | 0.093112 | Egg Recipes | 0.063881 | 0.572323 | 1.11E−42 |
| 2-arachidonoyl-GPE | Chicken Soup | | Freq | | | |
| (P-18:0/20:4)* | Freq | | | | | |
| X - 21339 | SF_Pita_wt | 0.062503 | SF_Hummus | 0.046357 | 0.56864 | 4.88E−42 |
| | | | Salad_wt | | | |
| 3-methylhistidine | SF_Salmon_wt | 0.054075 | Turkey | 0.046941 | 0.566964 | 9.51E−42 |
| | | | Meatballs, | | | |
| | | | Beef, Chicken | | | |
| | | | Freq | | | |
| X - 23649 | Ice Cream or | −0.0848 | Fresh | 0.055651 | 0.564258 | 2.77E−41 |
| | Popsicle which | | Vegetable | | | |
| | contains | | Salad With | | | |
| | Dairy Freq | | Dressing or | | | |
| | | | Oil Freq | | | |
| 4-ethylcatechol | Ice Cream or | −0.03467 | SF_Wine_wt | 0.031844 | 0.564161 | 2.88E−41 |
| sulfate | Popsicle which | | | | | |
| | contains | | | | | |
| | Dairy Freq | | | | | |
| X - 11880 | Salty Snacks | 0.073951 | SF_Coffee_wt | −0.06127 | 0.560703 | 1.11E−40 |
| | Freq | | | | | |
| X - 11308 | Beer Freq | 0.076051 | SF_Water_wt | −0.06578 | 0.560464 | 1.22E−40 |
| 2,3-dihydroxypyridine | SF_Cappuccino_wt | 0.047721 | Beef or | 0.042644 | 0.559514 | 1.76E−40 |
| | | | Chicken Soup | | | |
| | | | Freq | | | |
| beta-cryptoxanthin | SF_Orange_wt | 0.098657 | SF_Almonds_wt | 0.058808 | 0.557779 | 3.45E−40 |
| X - 13844 | Carrots, Fresh | 0.106691 | SF_Cappuccino_wt | 0.075613 | 0.557547 | 3.77E−40 |
| | or Cooked, | | | | | |
| | Carrot Juice | | | | | |
| | Freq | | | | | |
| X - 11372 | Oil as an | −0.07006 | SF_WhiteWheat_g_wt | 0.058058 | 0.556573 | 5.47E−40 |
| | addition for | | | | | |
| | Salads or | | | | | |
| | Stews Freq | | | | | |
| 1-palmitoyl-2- | SF_Tahini_wt | −0.05173 | Tahini Salad | −0.0373 | 0.540962 | 1.86E−37 |
| docosahexaenoyl-GPC | | | Freq | | | |
| (16:0/22:6) | | | | | | |
| X - 24949 | Beer Freq | 0.055873 | SF_Tzfatit | −0.0452 | 0.536216 | 1.03E−36 |
| | | | Cheese_wt | | | |
| X - 18914 | SF_Cottage | 0.065682 | SF_Olive | −0.05659 | 0.534199 | 2.11E−36 |
| | cheese_wt | | oil_wt | | | |
| X - 21661 | SF_Hummus | 0.059876 | Beer Freq | 0.057561 | 0.529844 | 9.82E−36 |
| | Salad_wt | | | | | |
| sphingomyelin | 5-9% White | 0.071071 | SF_Milk_wt | 0.069379 | 0.528447 | 1.60E−35 |
| (d17:1/16:0, | Cheese, | | | | | |
| d18:1/15:0, | Cottage Freq | | | | | |
| d16:1/17:0)* | | | | | | |
| X - 21752 | Internal | −0.06466 | SF_Natural | 0.056562 | 0.525177 | 4.97E−35 |
| | Organs Freq | | Yogurt_wt | | | |
| X - 12816 | Fries Freq | −0.09704 | Fresh | 0.059619 | 0.521149 | 1.98E−34 |
| | | | Vegetable | | | |
| | | | Salad With | | | |
| | | | Dressing or | | | |
| | | | Oil Freq | | | |
| 5alpha-androstan- | SF_Vegetable | 0.08957 | SF_Potatoes_wt | 0.069328 | 0.519896 | 3.03E−34 |
| 3alpha,17beta-diol | Salad_wt | | | | | |
| monosulfate (2) | | | | | | |
| stachydrine | SF_Mandarin_wt | 0.054964 | Orange or | 0.046129 | 0.518796 | 4.39E−34 |
| | | | Grapefruit | | | |
| | | | Freq | | | |
| X - 23639 | SF_Wine_wt | 0.047004 | SF_Rice | 0.045695 | 0.517285 | 7.30E−34 |
| | | | crackers_wt | | | |
| sphingomyelin | Falafel in Pita | −0.06567 | Hummus | −0.06423 | 0.513521 | 2.57E−33 |
| (d18:1/17:0, | Bread Freq | | Salad Freq | | | |
| d17:1/18:0, | | | | | | |
| d19:1/16:0) | | | | | | |
| X - 11381 | Pastrami or | 0.050351 | Beef, Veal, | 0.047009 | 0.510043 | 8.08E−33 |
| | Smoked Turkey | | Lamb, Pork, | | | |
| | Breast Freq | | Steak, Golash | | | |
| | | | Freq | | | |
| X - 24637 | SF_Pullet_wt | 0.01826 | Popsicle | 0.01729 | 0.509643 | 9.21E−33 |
| | | | Without Dairy | | | |
| | | | Freq | | | |
| X - 17185 | SF_WhiteWheat_g_wt | −0.05965 | SF_Bread_wt | −0.0539 | 0.508287 | 1.44E−32 |
| 5-acetylamino-6- | SF_Wine_wt | 0.041202 | SF_Walnuts_wt | −0.03496 | 0.507583 | 1.80E−32 |
| formylamino- | | | | | | |
| 3-methyluracil | | | | | | |
| X - 17145 | Dried Fruits | 0.071214 | White or | −0.05247 | 0.503748 | 6.24E−32 |
| | Freq | | Brown Sugar | | | |
| | | | Freq | | | |
| X - 11847 | SF_Hummus | 0.068976 | SF_Vegetable | 0.054953 | 0.502148 | 1.04E−31 |
| | Salad_wt | | Salad_wt | | | |
| 1,5-anhydroglucitol | SF_Apple_wt | −0.07966 | SF_Potatoes_wt | 0.058481 | 0.500762 | 1.62E−31 |
| (1,5-AG) | | | | | | |
| X - 18249 | Pastrami or | 0.067435 | SF_Milk_wt | 0.063078 | 0.49921 | 2.65E−31 |
| | Smoked Turkey | | | | | |
| | Breast Freq | | | | | |
| citraconate/ | 0.5-3% White | 0.046264 | SF_Cappuccino_wt | 0.039676 | 0.497666 | 4.31E−31 |
| glutaconate | Cheese, | | | | | |
| | Cottage Freq | | | | | |
| X - 12329 | Ice Cream or | −0.05105 | SF_Chicken | 0.032979 | 0.497287 | 4.86E−31 |
| | Popsicle which | | soup_wt | | | |
| | contains | | | | | |
| | Dairy Freq | | | | | |
| sphingomyelin | 3% Milk Freq | 0.065105 | Salty Cheese, | 0.062947 | 0.492709 | 2.03E−30 |
| (d18:1/19:0, | | | Tzfatit, | | | |
| d19:1/18:0)* | | | Bulgarian, | | | |
| | | | Brinza, Thick | | | |
| | | | Slice Freq | | | |
| X - 14939 | SF_Cappuccino_wt | −0.05836 | Processed | 0.048595 | 0.487672 | 9.54E−30 |
| | | | Meat Free | | | |
| | | | Products Freq | | | |
| acesulfame | SF_Diet | 0.074192 | SF_Diet Fruit | 0.062933 | 0.483786 | 3.09E−29 |
| | Coke_wt | | Drink_wt | | | |
| 1-stearoyl-2- | Jachnun, | −0.04281 | SF_Dark | 0.034822 | 0.483211 | 3.68E−29 |
| docosahexaenoyl-GPC | Mlawah, | | Chocolate_wt | | | |
| (18:0/22:6) | Kubana, | | | | | |
| | Cigars Freq | | | | | |
| 5alpha-androstan- | SF_Milk_wt | −0.0609 | SF_Vegetable | 0.05559 | 0.482126 | 5.09E−29 |
| 3alpha,17beta- | | | Salad_wt | | | |
| diol disulfate | | | | | | |
| tryptophan | 5-9% White | −0.05 | Peanuts Freq | 0.036135 | 0.478678 | 1.42E−28 |
| betaine | Cheese, | | | | | |
| | Cottage Freq | | | | | |
| gamma- | Pastrami or | 0.061609 | Tahini Salad | −0.04992 | 0.478459 | 1.51E−28 |
| glutamylvaline | Smoked Turkey | | Freq | | | |
| | Breast Freq | | | | | |
| daidzein | SF_Rice_wt | −0.01795 | Zucchini or | 0.014287 | 0.475213 | 3.93E−28 |
| sulfate (2) | | | Eggplant Freq | | | |
| sphingomyelin | Butter Freq | 0.081809 | SF_Dried | −0.06003 | 0.473984 | 5.63E−28 |
| (d18:1/25:0, | | | dates_wt | | | |
| d19:0/24:1, | | | | | | |
| d20:1/23:0, | | | | | | |
| d19:1/24:0)* | | | | | | |
| sphingomyelin | 5-9% White | 0.061768 | 3% Milk Freq | 0.06142 | 0.472036 | 9.90E−28 |
| (d18:1/14:0, | Cheese, | | | | | |
| d16:1/16:0)* | Cottage Freq | | | | | |
| X - 24475 | SF_Grapes_wt | 0.058532 | SF_WhiteWheat_g_wt | −0.05048 | 0.47104 | 1.32E−27 |
| methyl | Mandarin or | 0.075145 | Apple Freq | 0.068622 | 0.469282 | 2.19E−27 |
| glucopyranoside | Clementine | | | | | |
| (alpha + beta) | Freq | | | | | |
| X - 11795 | Turkey | −0.09559 | SF_Apple_wt | 0.051731 | 0.468553 | 2.70E−27 |
| | Meatballs, | | | | | |
| | Beef, Chicken | | | | | |
| | Freq | | | | | |
| docosahexaenoate | Simple | −0.06249 | SF_Salmon_wt | 0.048854 | 0.463115 | 1.26E−26 |
| (DHA; 22:6n3) | Cookies or | | | | | |
| | Biscuits Freq | | | | | |
| X - 11849 | SF_Wine_wt | 0.04672 | Grapes or | 0.044163 | 0.460034 | 2.98E−26 |
| | | | Raisins Freq | | | |
| X - 18922 | Artificial | −0.06254 | Tahini Salad | 0.054076 | 0.45954 | 3.42E−26 |
| | Sweeteners | | Freq | | | |
| | Freq | | | | | |
| S-methylcysteine | SF_Potatoes_wt | −0.04241 | SF_Lentils_wt | 0.032798 | 0.459207 | 3.75E−26 |
| sulfoxide | | | | | | |
| perfluorooctane- | Chicken or | 0.048935 | Herbal Tea | −0.044 | 0.453276 | 1.91E−25 |
| sulfonic acid | Turkey With | | Freq | | | |
| (PFOS) | Skin Freq | | | | | |
| 3-hydroxystachydrine* | Orange or | 0.075756 | Orange or | 0.068873 | 0.452535 | 2.34E−25 |
| | Grapefruit | | Grapefruit | | | |
| | Freq | | Juice Freq | | | |
| sphingomyelin | SF_Tahini_wt | −0.06725 | Cooked | −0.06432 | 0.451541 | 3.06E−25 |
| (d18:2/23:1)* | | | Legumes Freq | | | |
| maleate | 0.5-3% White | 0.031706 | Milk or Dark | 0.027896 | 0.4509 | 3.64E−25 |
| | Cheese, | | Chocolate | | | |
| | Cottage Freq | | Freq | | | |
| eicosenedioate | SF_Dark | −0.06387 | SF_Butter_wt | −0.05514 | 0.442637 | 3.29E−24 |
| (C20:1-DC)* | Chocolate_wt | | | | | |
| homostachydrine* | Potatoes | −0.06012 | SF_Cucumber_wt | 0.0464 | 0.440766 | 5.37E−24 |
| | Boiled, | | | | | |
| | Baked, | | | | | |
| | Mashed, | | | | | |
| | Potatoes | | | | | |
| | Salad Freq | | | | | |
| creatine | SF_Onion_wt | −0.05296 | SF_Vegetable | −0.04866 | 0.440324 | 6.02E−24 |
| | | | Salad_wt | | | |
| X - 17653 | Cooked | 0.055492 | SF_Egg_wt | −0.0525 | 0.434164 | 2.95E−23 |
| | Legumes Freq | | | | | |
| catechol | Wholemeal or | 0.052213 | Red Pepper | 0.045469 | 0.431796 | 5.39E−23 |
| sulfate | Rye Bread | | Freq | | | |
| | Freq | | | | | |
| X - 16935 | Artificial | −0.08278 | SF_WholeWheat_g_wt | −0.06553 | 0.431754 | 5.44E−23 |
| | Sweeteners | | | | | |
| | Freq | | | | | |
| sphingomyelin | SF_Milk_wt | 0.062056 | SF_Tomatoes_wt | −0.05112 | 0.429145 | 1.05E−22 |
| (d18:2/21:0, | | | | | | |
| d16:2/23:0)* | | | | | | |
| sphingomyelin | Hummus | −0.05922 | 5-9% White | 0.057783 | 0.428361 | 1.28E−22 |
| (d17:2/16:0, | Salad Freq | | Cheese, | | | |
| d18:2/15:0)* | | | Cottage Freq | | | |
| S-methylcysteine | Fresh | 0.04016 | SF_Cooked | 0.038905 | 0.427534 | 1.57E−22 |
| | Vegetable | | cauliflower_wt | | | |
| | Salad With | | | | | |
| | Dressing or | | | | | |
| | Oil Freq | | | | | |
| N-(2-furoyl)glycine | SF_Fried | −0.03011 | Ice Cream or | −0.02755 | 0.425786 | 2.43E−22 |
| | onions_wt | | Popsicle which | | | |
| | | | contains | | | |
| | | | Dairy Freq | | | |
| 2,6-dihydroxybenzoic | Pastrami or | −0.05537 | Sweet Dry | 0.054464 | 0.425667 | 2.50E−22 |
| acid | Smoked Turkey | | Wine, | | | |
| | Breast Freq | | Cocktails Freq | | | |
| X - 12837 | SF_Olive | 0.057798 | Pear Fresh, | −0.04673 | 0.423939 | 3.84E−22 |
| | oil_wt | | Cooked or | | | |
| | | | Canned Freq | | | |
| pyroglutamine* | Turkey | −0.04632 | Regular Sodas | 0.045205 | 0.422804 | 5.08E−22 |
| | Meatballs, | | with Sugar | | | |
| | Beef, Chicken | | Freq | | | |
| | Freq | | | | | |
| N-delta- | SF_Avocado_wt | 0.059246 | SF_Vegetable | 0.044284 | 0.422436 | 5.56E−22 |
| acetylornithine | | | Salad_wt | | | |
| X - 21736 | Carrots, Fresh | −0.05267 | SF_Olives_wt | 0.052412 | 0.422216 | 5.87E−22 |
| | or Cooked, | | | | | |
| | Carrot Juice | | | | | |
| | Freq | | | | | |
| tridecenedioate | SF_Milk_wt | 0.079938 | Butter Freq | 0.064098 | 0.421426 | 7.12E−22 |
| (C13:1-DC)* | | | | | | |
| heneicosa- | Simple | −0.06039 | 0.5-3% White | 0.059638 | 0.420245 | 9.50E−22 |
| pentaenoate | Cookies or | | Cheese, | | | |
| (21:5n3) | Biscuits Freq | | Cottage Freq | | | |
| 2-aminobutyrate | Olives Freq | 0.046964 | Canned Tuna | 0.043994 | 0.420111 | 9.82E−22 |
| | | | or Tuna Salad | | | |
| | | | Freq | | | |
| X - 11378 | Oil as an | −0.07381 | Chicken or | −0.06575 | 0.419887 | 1.04E−21 |
| | addition for | | Turkey | | | |
| | Salads or | | Without Skin | | | |
| | Stews Freq | | Freq | | | |
| 2-hydroxylaurate | SF_Coffee_wt | −0.05685 | SF_WhiteWheat_g_wt | 0.041425 | 0.418576 | 1.43E−21 |
| 17-methylstearate | SF_Butter_wt | 0.042047 | Coated or | −0.03047 | 0.418485 | 1.46E−21 |
| | | | Stuffed | | | |
| | | | Cookies, | | | |
| | | | Waffles or | | | |
| | | | Biscuits Freq | | | |
| 15-methylpalmitate | SF_Tahini_wt | −0.05704 | Cooked | −0.05242 | 0.417042 | 2.07E−21 |
| | | | Legumes Freq | | | |
| sphingomyelin | Egg Recipes | −0.06736 | Sour Cream | 0.064164 | 0.415691 | 2.86E−21 |
| (d18:2/14:0, | Freq | | Freq | | | |
| d18:1/14:l)* | | | | | | |
| hippurate | SF_Potatoes_wt | −0.03792 | Regular Sodas | −0.03789 | 0.415062 | 3.32E−21 |
| | | | with Sugar | | | |
| | | | Freq | | | |
| X - 12730 | Ice Cream or | −0.03589 | SF_Salmon_wt | −0.03151 | 0.413674 | 4.63E−21 |
| | Popsicle which | | | | | |
| | contains | | | | | |
| | Dairy Freq | | | | | |
| 1-(1-enyl-palmitoyl)- | Beef or | 0.049868 | Chicken or | 0.047927 | 0.411153 | 8.43E−21 |
| 2-arachidonoyl-GPC | Chicken Soup | | Turkey | | | |
| (P-16:0/20:4)* | Freq | | Without Skin | | | |
| | | | Freq | | | |
| caffeic acid | Ice Cream or | −0.04549 | SF_Wholemeal | 0.041568 | 0.408155 | 1.71E−20 |
| sulfate | Popsicle which | | Bread_wt | | | |
| | contains | | | | | |
| | Dairy Freq | | | | | |
| 1-(1-enyl- | >=16% Yellow | 0.05373 | SF_Dark | 0.048545 | 0.406098 | 2.76E−20 |
| stearoyl)-GPE | Cheese Freq | | Chocolate_wt | | | |
| (P-18:0)* | | | | | | |
| 3-methyl catechol | SF_Natural | 0.063031 | SF_Bread_wt | −0.04462 | 0.405559 | 3.12E−20 |
| sulfate (2) | Yogurt_wt | | | | | |
| oxalate | SF_WhiteWheat_g_wt | −0.05553 | SF_Meatballs_wt | −0.05017 | 0.405418 | 3.23E−20 |
| (ethanedioate) | | | | | | |
| eicosapentaenoate | Simple | −0.07017 | 0.5-3% White | 0.043958 | 0.40463 | 3.87E−20 |
| (EPA; 20:5n3) | Cookies or | | Cheese, | | | |
| | Biscuits Freq | | Cottage Freq | | | |
| X - 12738 | SF_Natural | 0.070084 | SF_Bread_wt | −0.04886 | 0.404165 | 4.31E−20 |
| | Yogurt_wt | | | | | |
| X - 21383 | SF_Olive | −0.04927 | Butter Freq | −0.04848 | 0.403785 | 4.71E−20 |
| | oil_wt | | | | | |
| creatinine | Alcoholic | 0.052069 | SF_Beer_wt | 0.045235 | 0.403706 | 4.80E−20 |
| | Drinks Freq | | | | | |
| gentisate | SF_Butter_wt | −0.05224 | Peas, Green | 0.045827 | 0.403106 | 5.51E−20 |
| | | | Beans or Okra | | | |
| | | | Cooked Freq | | | |
| X - 24951 | Alcoholic | 0.06119 | SF_Brown | −0.04641 | 0.402968 | 5.68E−20 |
| | Drinks Freq | | Rice_wt | | | |
| X - 17654 | Alcoholic | 0.067916 | SF_WholeWheat_g_wt | 0.052957 | 0.402542 | 6.27E−20 |
| | Drinks Freq | | | | | |
| tiglylcarnitine | SF_Natural | 0.057641 | Chicken or | 0.055828 | 0.40231 | 6.61E−20 |
| (C5:1-DC) | Yogurt_wt | | Turkey With | | | |
| | | | Skin Freq | | | |
| 2-aminoheptanoate | 3% Milk Freq | −0.05284 | SF_Cottage | −0.04291 | 0.398862 | 1.45E−19 |
| | | | cheese_wt | | | |
| phytanate | SF_Butter_wt | 0.053456 | 5-9% White | 0.050306 | 0.397034 | 2.19E−19 |
| | | | Cheese, | | | |
| | | | Cottage Freq | | | |
| androsterone | Beef, Veal, | 0.045773 | SF_Beer_wt | 0.043302 | 0.396289 | 2.60E−19 |
| glucuronide | Lamb, Pork, | | | | | |
| | Steak, Golash | | | | | |
| | Freq | | | | | |
| 4-vinylguaiacol | SF_Salmon_wt | −0.08545 | SF_Wholemeal | 0.0801 | 0.395545 | 3.07E−19 |
| sulfate | | | Bread_wt | | | |
| 1-docosahexaenoyl- | Pita Freq | −0.05528 | SF_Salmon_wt | 0.044281 | 0.395373 | 3.19E−19 |
| glycerol (22:6) | | | | | | |
| 2-aminophenol | SF_Cereals_wt | 0.074577 | Wholemeal or | 0.049861 | 0.394949 | 3.51E−19 |
| sulfate | | | Rye Bread | | | |
| | | | Freq | | | |
| N2,N5-diacetylornithine | SF_Brown | 0.051766 | SF_Quinoa_wt | 0.051267 | 0.394696 | 3.71E−19 |
| | Rice_wt | | | | | |
| X - 17676 | Regular Tea | −0.06549 | SF_WhiteWheat_g_wt | −0.04848 | 0.393674 | 4.66E−19 |
| | Freq | | | | | |
| carotene diol (2) | SF_Potatoes_wt | −0.04741 | Fresh | 0.044641 | 0.392248 | 6.40E−19 |
| | | | Vegetable | | | |
| | | | Salad With | | | |
| | | | Dressing or | | | |
| | | | Oil Freq | | | |
| 4-ethylphenylsulfate | SF_Wine_wt | 0.03036 | Roll or | −0.02981 | 0.391656 | 7.30E−19 |
| | | | Bageles Freq | | | |
| 2-aminoadipate | Artificial | 0.04248 | SF_Tea_wt | −0.04163 | 0.390848 | 8.72E−19 |
| | Sweeteners | | | | | |
| | Freq | | | | | |
| O-methylcatechol | Avocado Freq | 0.03663 | SF_WholeWheat_g_wt | 0.035324 | 0.390244 | 9.96E−19 |
| sulfate | | | | | | |
| X - 24655 | SF_Sushi_wt | 0.014172 | Cooked | 0.012424 | 0.388101 | 1.59E−18 |
| | | | Legumes Freq | | | |
| ceramide | 5-9% White | 0.071636 | >=16% Yellow | 0.069378 | 0.387214 | 1.94E−18 |
| (d18:1/14:0, | Cheese, | | Cheese Freq | | | |
| d16:1/16:0)* | Cottage Freq | | | | | |
| X - 17325 | Fried Fish | −0.03827 | Egg Recipes | −0.03532 | 0.383891 | 3.98E−18 |
| | Freq | | Freq | | | |
| N1-Methyl-2-pyridone- | Chicken or | 0.04899 | SF_Salmon_wt | 0.048479 | 0.383476 | 4.35E−18 |
| 5-carboxamide | Turkey | | | | | |
| | Without Skin | | | | | |
| | Freq | | | | | |
| urate | SF_Milk_wt | −0.05192 | SF_Potatoes_wt | 0.044827 | 0.382399 | 5.48E−18 |
| carotene diol (3) | SF_WhiteWheat_g_wt | −0.02943 | SF_Vegetable | 0.027763 | 0.379593 | 9.97E−18 |
| | | | Salad_wt | | | |
| 1-methylhistidine | Processed | −0.05225 | Pastrami or | 0.051223 | 0.377947 | 1.41E−17 |
| | Meat Free | | Smoked Turkey | | | |
| | Products Freq | | Breast Freq | | | |
| 3-acetylphenol | SF_Cappuccino_wt | 0.031901 | SF_Wine_wt | 0.023843 | 0.37676 | 1.81E−17 |
| sulfate | | | | | | |
| theobromine | SF_Dark | 0.074842 | Beef or | −0.0264 | 0.375799 | 2.22E−17 |
| | Chocolate_wt | | Chicken Soup | | | |
| | | | Freq | | | |
| N-methylproline | SF_Mandarin | 0.039602 | SF_Vegetable | 0.029372 | 0.375553 | 2.34E−17 |
| | _wt | | Salad_wt | | | |
| dihydrocaffeate | Green Pepper | 0.038352 | Lettuce Freq | 0.03572 | 0.370926 | 6.11E−17 |
| sulfate (2) | Freq | | | | | |
| threonate | SF_Butter_wt | −0.05228 | Turkey | −0.0474 | 0.370249 | 7.03E−17 |
| | | | Meatballs, | | | |
| | | | Beef, Chicken | | | |
| | | | Freq | | | |
| X - 12221 | SF_Bread_wt | −0.06191 | Coffee Freq | 0.060087 | 0.369124 | 8.85E−17 |
| myristoyl | Beer Freq | −0.04834 | 0.5-3% White | 0.047404 | 0.367845 | 1.15E−16 |
| dihydrosphingo- | | | Cheese, | | | |
| myelin | | | Cottage Freq | | | |
| (d18:0/14:0)* | | | | | | |
| X - 17367 | Potatoes | −0.04261 | Egg Recipes | −0.04036 | 0.367768 | 1.17E−16 |
| | Boiled, | | Freq | | | |
| | Baked, | | | | | |
| | Mashed, | | | | | |
| | Potatoes | | | | | |
| | Salad Freq | | | | | |
| 4-methyl-2- | White or | −0.04939 | Olives Freq | 0.04369 | 0.366956 | 1.38E−16 |
| oxopentanoate | Brown Sugar | | | | | |
| | Freq | | | | | |
| 1-myristoyl-2- | SF_Tahini_wt | −0.06294 | SF_WhiteWheat_g_wt | 0.05446 | 0.365079 | 2.01E−16 |
| palmitoyl-GPC | | | | | | |
| (14:0/16:0) | | | | | | |
| arabonate/xylonate | SF_Rice | 0.041739 | Beef, Veal, | −0.03616 | 0.364071 | 2.47E−16 |
| | crackers_wt | | Lamb, Pork, | | | |
| | | | Steak, Golash | | | |
| | | | Freq | | | |
| leucine | Beef, Veal, | 0.039325 | Dried Fruits | −0.03856 | 0.363323 | 2.87E−16 |
| | Lamb, Pork, | | Freq | | | |
| | Steak, Golash | | | | | |
| | Freq | | | | | |
| 5alpha-androstan- | SF_Beer_wt | 0.059412 | SF_Pita_wt | 0.038293 | 0.3628 | 3.19E−16 |
| 3beta,17beta- | | | | | | |
| diol disulfate | | | | | | |
| 3-methylxanthine | SF_Dark | 0.103324 | Beef or | −0.05476 | 0.360778 | 4.77E−16 |
| | Chocolate_wt | | Chicken Soup | | | |
| | | | Freq | | | |
| X - 16087 | White or | −0.06196 | Simple | −0.05461 | 0.360488 | 5.05E−16 |
| | Brown Sugar | | Cookies or | | | |
| | Freq | | Biscuits Freq | | | |
| 3-methyl-2- | SF_Beef_wt | 0.052248 | Olives Freq | 0.039103 | 0.359631 | 5.99E−16 |
| oxovalerate | | | | | | |
| 2-hydroxybutyrate/ | SF_Cereals_wt | −0.04741 | Fish (not | 0.043758 | 0.358057 | 8.17E−16 |
| 2-hydroxyisobutyrate | | | Tuna) Pickled, | | | |
| | | | Dried, Smoked, | | | |
| | | | Canned Freq | | | |
| ergothioneine | SF_Schnitzel_wt | −0.04274 | Fresh | 0.042489 | 0.357018 | 1.00E−15 |
| | | | Vegetable | | | |
| | | | Salad With | | | |
| | | | Dressing or | | | |
| | | | Oil Freq | | | |
| 1-lignoceroyl-GPC | Roll or | −0.06077 | Peanuts Freq | 0.056537 | 0.356222 | 1.17E−15 |
| (24:0) | Bageles Freq | | | | | |
| linoleoylcarnitine | Beer Freq | 0.059653 | Hummus | 0.053653 | 0.355664 | 1.31E−15 |
| (C18:2)* | | | Salad Freq | | | |
| N-acetylcarnosine | Wholemeal or | −0.05137 | SF_Beef_wt | 0.048939 | 0.355399 | 1.38E−15 |
| | Rye Bread | | | | | |
| | Freq | | | | | |
| N-trimethyl 5- | SF_Coffee_wt | 0.041994 | Salty Cheese, | 0.041497 | 0.354891 | 1.52E−15 |
| aminovalerate | | | Tzfatit, | | | |
| | | | Bulgarian, | | | |
| | | | Brinza, Thick | | | |
| | | | Slice Freq | | | |
| sphingomyelin | Salty Cheese, | 0.051802 | Beer Freq | −0.04486 | 0.354618 | 1.60E−15 |
| (d18:1/22:2, | Tzfatit, | | | | | |
| d18:2/22:1, | Bulgarian, | | | | | |
| d16:1/24:2)* | Brinza, | | | | | |
| | Medium Slice | | | | | |
| | Freq | | | | | |
| urea | Cooked | −0.04419 | SF_Natural | 0.043918 | 0.354161 | 1.75E−15 |
| | Cereal such as | | Yogurt_wt | | | |
| | Oatmeal | | | | | |
| | Porridge Freq | | | | | |
| 3-carboxy-4- | Fresh | 0.050826 | Sausages | 0.048362 | 0.352926 | 2.23E−15 |
| methyl-5- | Vegetable | | such as | | | |
| pentyl-2- | Salad With | | Salami Freq | | | |
| furanpropionate | Dressing or | | | | | |
| (3-CMPFP)** | Oil Freq | | | | | |
| Fibrinopeptide | Processed | −0.01278 | SF_Tahini_wt | −0.01213 | 0.352507 | 2.41E−15 |
| A(7-16)* | Meat Free | | | | | |
| | Products Freq | | | | | |
| 3-(4-hydroxy- | 5-9% Yellow | 0.052535 | Cooked | −0.04648 | 0.351439 | 2.96E−15 |
| phenyl)lactate | Cheese Freq | | Cereal such as | | | |
| | | | Oatmeal | | | |
| | | | Porridge Freq | | | |
| 1-(1-enyl- | Egg, Hard | 0.055643 | Processed | −0.04832 | 0.351396 | 2.99E−15 |
| palmitoyl)-2- | Boiled or Soft | | Meat Free | | | |
| linoleoyl-GPE | Freq | | Products Freq | | | |
| (P-16:0/18:2)* | | | | | | |
| X - 24948 | Canned Tuna | 0.043396 | SF_WhiteWheat_g_wt | 0.043345 | 0.351043 | 3.20E−15 |
| | or Tuna Salad | | | | | |
| | Freq | | | | | |
| 1-(1-enyl-stearoyl)- | SF_WhiteWheat_g_wt | −0.04951 | SF_Onion_wt | −0.04387 | 0.350153 | 3.79E−15 |
| 2-oleoyl-GPE | | | | | | |
| (P-18:0/18:1) | | | | | | |
| 3-hydroxybutyryl- | Olives Freq | 0.053049 | Wholemeal or | −0.04305 | 0.349047 | 4.69E−15 |
| carnitine (1) | | | Rye Bread | | | |
| | | | Freq | | | |
| X - 19183 | Avocado Freq | 0.036242 | Coffee Freq | −0.03184 | 0.348713 | 5.00E−15 |
| X - 23659 | Cooked | 0.039292 | 3% Milk Freq | −0.03427 | 0.34782 | 5.92E−15 |
| | Legumes Freq | | | | | |
| 7-methylurate | SF_Dark | 0.048154 | SF_Lentils_wt | −0.03192 | 0.347816 | 5.93E−15 |
| | Chocolate_wt | | | | | |
| X - 24757 | Carrots, Fresh | 0.051412 | Coffee Freq | 0.046139 | 0.347733 | 6.02E−15 |
| | or Cooked, | | | | | |
| | Carrot Juice | | | | | |
| | Freq | | | | | |
| X - 24328 | Beer Freq | 0.053836 | SF_Hummus | 0.052723 | 0.347635 | 6.13E−15 |
| | | | Salad_wt | | | |
| pregn steroid | SF_Rice | −0.04032 | SF_Beer_wt | 0.039884 | 0.346265 | 7.95E−15 |
| monosulfate | crackers_wt | | | | | |
| C21H34O5S* | | | | | | |
| ethyl | Parsley, | 0.016401 | SF_Pita_wt | 0.015055 | 0.34588 | 8.55E−15 |
| glucuronide | Celery, | | | | | |
| | Fennel, Dill, | | | | | |
| | Cilantro, | | | | | |
| | Green Onion | | | | | |
| | Freq | | | | | |
| 3-hydroxyhippurate | SF_Wholemeal | 0.041614 | SF_Jam_wt | 0.041016 | 0.345164 | 9.78E−15 |
| sulfate | Crackers_wt | | | | | |
| 7-methylxanthine | SF_Coffee_wt | 0.075186 | Beef or | −0.05062 | 0.34498 | 1.01E−14 |
| | | | Chicken Soup | | | |
| | | | Freq | | | |
| X - 18886 | Peach, | −0.05565 | SF_Olives_wt | 0.05243 | 0.344884 | 1.03E−14 |
| | Nectarine, | | | | | |
| | Plum Freq | | | | | |
| glycine | SF_Diet | −0.05595 | Cucumber | −0.04961 | 0.344431 | 1.12E−14 |
| conjugate of | Coke_wt | | Freq | | | |
| C10H14O2 (1)* | | | | | | |
| caprate (10:0) | 3% Milk Freq | 0.052666 | SF_Tahini_wt | −0.04831 | 0.343847 | 1.25E−14 |
| dihydroferulic | SF_Wholemeal | 0.078961 | Sugar | −0.06382 | 0.342386 | 1.65E−14 |
| acid | Bread_wt | | Sweetened | | | |
| | | | Chocolate | | | |
| | | | Milk Freq | | | |
| X - 12306 | SF_Hummus_wt | 0.05638 | Apple Freq | 0.05467 | 0.342141 | 1.72E−14 |
| leucylalanine | Sausages Freq | −0.02017 | SF_WholeWheat_g_wt | −0.00951 | 0.339579 | 2.77E−14 |
| N1-methylinosine | SF_Soymilk_wt | −0.03325 | Orange or | −0.02788 | 0.339464 | 2.83E−14 |
| | | | Grapefruit | | | |
| | | | Juice Freq | | | |
| X - 12544 | SF_WhiteWheat_g_wt | 0.052027 | SF_Tea_wt | 0.047978 | 0.339085 | 3.03E−14 |
| androstenediol | Lemon Freq | −0.04738 | SF_Pita_wt | 0.040231 | 0.337347 | 4.17E−14 |
| (3alpha,17alpha) | | | | | | |
| monosulfate (3) | | | | | | |
| argininate* | SF_Apple_wt | 0.036457 | SF_Lentils_wt | 0.033548 | 0.337039 | 4.41E−14 |
| ferulic acid 4- | SF_Bread_wt | −0.05366 | Jachnun, | −0.02485 | 0.336545 | 4.83E−14 |
| sulfate | | | Mlawah, | | | |
| | | | Kubana, | | | |
| | | | Cigars Freq | | | |
| pregnen-diol | Lemon Freq | −0.02386 | Coffee Freq | −0.02354 | 0.334663 | 6.80E−14 |
| disulfate | | | | | | |
| C21H34O8S2* | | | | | | |
| N-acetyl-3- | Pastrami or | 0.064586 | 0.5-3% White | 0.055006 | 0.334575 | 6.91E−14 |
| methylhistidine* | Smoked Turkey | | Cheese, | | | |
| | Breast Freq | | Cottage Freq | | | |
| X - 17655 | SF_Vegetable | 0.054329 | SF_Milk_wt | −0.04166 | 0.334257 | 7.32E−14 |
| | Salad_wt | | | | | |
| X - 24693 | Zucchini or | 0.051715 | Vegetable | 0.046437 | 0.334182 | 7.42E−14 |
| | Eggplant Freq | | Soup Freq | | | |
| S-methylmethionine | SF_Apple_wt | 0.064711 | SF_Milk_wt | −0.06431 | 0.332853 | 9.43E−14 |
| X - 23314 | SF_Wine_wt | 0.041034 | Orange or | 0.037983 | 0.331446 | 1.21E−13 |
| | | | Grapefruit | | | |
| | | | Freq | | | |
| sphingomyelin | White or | 0.039429 | Beef, Veal, | −0.03463 | 0.330379 | 1.47E−13 |
| (d18:1/20:2, | Brown Sugar | | Lamb, Pork, | | | |
| d18:2/20:1, | Freq | | Steak, Golash | | | |
| d16:1/22:2)* | | | Freq | | | |
| androstenediol | SF_Carrots_wt | 0.052597 | SF_Rice | −0.05118 | 0.329787 | 1.63E−13 |
| (3alpha,17alpha) | | | crackers_wt | | | |
| monosulfate (2) | | | | | | |
| alpha-hydroxy- | Sausages Freq | 0.026157 | SF_WhiteWheat_g_wt | 0.026134 | 0.329164 | 1.82E−13 |
| isocaproate | | | | | | |
| X - 24473 | 3% Milk Freq | −0.04528 | SF_Grapes_wt | 0.042605 | 0.329097 | 1.84E−13 |
| X - 24337 | Regular Sodas | 0.05234 | Fresh | −0.05186 | 0.328477 | 2.06E−13 |
| | with Sugar | | Vegetable | | | |
| | Freq | | Salad Without | | | |
| | | | Dressing or | | | |
| | | | Oil Freq | | | |
| X - 21829 | SF_WhiteWheat_g_wt | −0.06322 | Olives Freq | 0.063048 | 0.327598 | 2.41E−13 |
| X - 23780 | Grapes or | −0.03421 | SF_Rice | 0.033074 | 0.327324 | 2.52E−13 |
| | Raisins Freq | | crackers_wt | | | |
| deoxycarnitine | Chicken or | 0.050117 | Shish Kebab | 0.045183 | 0.325373 | 3.56E−13 |
| | Turkey With | | in Pita Bread | | | |
| | Skin Freq | | Freq | | | |
| N,N,N-trimethyl- | Falafel in Pita | 0.047549 | Beer Freq | 0.046313 | 0.325337 | 3.58E−13 |
| alanylproline | Bread Freq | | | | | |
| betaine (TMAP) | | | | | | |
| Fibrinopeptide | SF_Roasted | −0.01936 | Cauliflower or | 0.018019 | 0.325192 | 3.67E−13 |
| B (1-13)** | eggplant_wt | | Broccoli Freq | | | |
| stearoylcarnitine | Couscous, | −0.04283 | Sausages | 0.033821 | 0.324693 | 4.01E−13 |
| (C18) | Burgul, | | such as | | | |
| | Mamaliga, | | Salami Freq | | | |
| | Groats Freq | | | | | |
| myristate (14:0) | Butter Freq | 0.039914 | Regular Sodas | −0.03953 | 0.324623 | 4.06E−13 |
| | | | with Sugar | | | |
| | | | Freq | | | |
| histidine | SF_Cereals_wt | 0.052599 | SF_Hummus | −0.04732 | 0.323506 | 4.93E−13 |
| | | | Salad_wt | | | |
| isovaleryl- | Egg Recipes | 0.048097 | Processed | −0.03721 | 0.322882 | 5.49E−13 |
| carnitine (C5) | Freq | | Meat Free | | | |
| | | | Products Freq | | | |
| X - 13431 | Fish (not | 0.049331 | Fish Cooked, | 0.047978 | 0.32243 | 5.94E−13 |
| | Tuna) Pickled, | | Baked or | | | |
| | Dried, Smoked, | | Grilled Freq | | | |
| | Canned Freq | | | | | |
| X - 13255 | SF_Rice | 0.059722 | SF_Yellow | 0.053973 | 0.320938 | 7.68E−13 |
| | crackers_wt | | Cheese_wt | | | |
| X - 21319 | Coated or | 0.04195 | SF_Cappuccino_wt | −0.03998 | 0.320446 | 8.36E−13 |
| | Stuffed | | | | | |
| | Cookies, | | | | | |
| | Waffles or | | | | | |
| | Biscuits Freq | | | | | |
| X - 13866 | Fish (not | 0.050089 | Beef or | 0.041531 | 0.320354 | 8.49E−13 |
| | Tuna) Pickled, | | Chicken Soup | | | |
| | Dried, Smoked, | | Freq | | | |
| | Canned Freq | | | | | |
| 3-methyl-2- | SF_Apple_wt | −0.02619 | SF_Coffee_wt | −0.02532 | 0.319895 | 9.19E−13 |
| oxobutyrate | | | | | | |
| X - 07765 | Coated or | 0.057179 | 3-4.5% | 0.037393 | 0.316208 | 1.72E−12 |
| | Stuffed | | Pudding, | | | |
| | Cookies, | | Cheese With | | | |
| | Waffles or | | Additions | | | |
| | Biscuits Freq | | Freq | | | |
| X - 22509 | SF_Apple_wt | 0.021902 | 3-4.5% | 0.0217 | 0.315541 | 1.93E−12 |
| | | | Lebben, Eshel | | | |
| | | | Freq | | | |
| 2,3-dihydroxy- | Cooked | 0.054212 | Pasta or | −0.04029 | 0.315044 | 2.10E−12 |
| 2-methylbutyrate | Vegetable | | Flakes Freq | | | |
| | Salads Freq | | | | | |
| ADpSGEGDFX | Vegetable | 0.031872 | SF_WhiteWheat_g_wt | −0.02536 | 0.314514 | 2.29E−12 |
| AEGGGVR* | Soup Freq | | | | | |
| 5alpha-androstan- | SF_Coffee_wt | −0.04871 | Shish Kebab | 0.043108 | 0.313926 | 2.53E−12 |
| 3alpha,17alpha- | | | in Pita Bread | | | |
| diol monosulfate | | | Freq | | | |
| X - 24832 | Beef, Veal, | 0.053751 | Dried Fruits | −0.04756 | 0.313391 | 2.77E−12 |
| | Lamb, Pork, | | Freq | | | |
| | Steak, Golash | | | | | |
| | Freq | | | | | |
| carotene diol | SF_Carrots_wt | 0.039772 | SF_Chicken | −0.03954 | 0.313221 | 2.85E−12 |
| (1) | | | breast_wt | | | |
| 2-methylserine | SF_WhiteWheat_g_wt | −0.0551 | Pear Fresh, | 0.046295 | 0.312658 | 3.13E−12 |
| | | | Cooked or | | | |
| | | | Canned Freq | | | |
| N-methylhydroxy- | SF_Mandarin_wt | 0.047633 | SF_Banana_wt | 0.026113 | 0.312648 | 3.13E−12 |
| proline** | | | | | | |
| catechol | SF_WhiteWheat_g_wt | −0.03101 | SF_Bread_wt | −0.02845 | 0.3126 | 3.16E−12 |
| glucuronide | | | | | | |
| 3-hydroxyhippurate | SF_WholeWheat_g_wt | 0.066147 | SF_Wholemeal | 0.043477 | 0.311264 | 3.95E−12 |
| | | | Bread_wt | | | |
| X - 18899 | 5-9% White | −0.06269 | SF_Olives_wt | −0.04944 | 0.310712 | 4.33E−12 |
| | Cheese, | | | | | |
| | Cottage Freq | | | | | |
| pregnenetriol | Coffee Freq | −0.02994 | SF_Apple_wt | −0.02991 | 0.310417 | 4.54E−12 |
| disulfate* | | | | | | |
| N-stearoyl- | SF_Avocado_wt | −0.05429 | Cooked | −0.04721 | 0.310022 | 4.85E−12 |
| sphingosine | | | Legumes Freq | | | |
| (d18:1/18:0)* | | | | | | |
| 10-undecenoate | SF_Milk_wt | 0.061107 | 3% Milk Freq | 0.052872 | 0.308837 | 5.90E−12 |
| (11:1n1) | | | | | | |
| X - 15503 | SF_Tomatoes_wt | −0.06039 | Alcoholic | −0.05866 | 0.308375 | 6.36E−12 |
| | | | Drinks Freq | | | |
| 1-palmitoyl-2- | Tahini Salad | −0.041 | Wholemeal | 0.036322 | 0.307733 | 7.07E−12 |
| palmitoleoyl-GPC | Freq | | Crackers Freq | | | |
| (16:0/16:1)* | | | | | | |
| X - 15486 | SF_Tomatoes_wt | −0.03777 | Herbal Tea | −0.03149 | 0.307631 | 7.19E−12 |
| | | | Freq | | | |
| gamma-tocopherol/ | SF_Hummus | 0.042454 | SF_Sugar Free | −0.04028 | 0.30664 | 8.45E−12 |
| beta-tocopherol | Salad_wt | | Gum_wt | | | |
| sphingomyelin | Salty Cheese, | 0.046175 | 0.5-3% White | 0.039202 | 0.305718 | 9.82E−12 |
| (d18:1/21:0, | Tzfatit, | | Cheese, | | | |
| d17:1/22:0, | Bulgarian, | | Cottage Freq | | | |
| d16:1/23:0)* | Brinza, Thick | | | | | |
| | Slice Freq | | | | | |
| 1-(1-enyl- | >=16% Yellow | 0.049138 | Beef, Veal, | 0.039788 | 0.305649 | 9.93E−12 |
| palmitoyl)-GPE | Cheese Freq | | Lamb, Pork, | | | |
| (P-16:0)* | | | Steak, Golash | | | |
| | | | Freq | | | |
| isobutyryl- | SF_Coffee_wt | 0.050446 | SF_Apple_wt | 0.03832 | 0.304365 | 1.22E−11 |
| carnitine (C4) | | | | | | |
| X - 18901 | SF_WhiteWheat_g_wt | −0.04052 | SF_Tea_wt | 0.037512 | 0.304305 | 1.24E−11 |
| gamma- | SF_Vegetable | 0.043006 | Banana Freq | −0.04033 | 0.304253 | 1.25E−11 |
| glutamylglutamate | Salad_wt | | | | | |
| X - 15492 | Falafel in Pita | 0.060056 | SF_Hummus | 0.05209 | 0.304004 | 1.30E−11 |
| | Bread Freq | | Salad_wt | | | |
| X - 16580 | SF_WhiteWheat_g_wt | −0.06138 | SF_Cooked | −0.04311 | 0.303593 | 1.39E−11 |
| | | | Sweet | | | |
| | | | potato_wt | | | |
| sphingomyelin | White or | 0.042974 | SF_Olive | −0.04157 | 0.303253 | 1.46E−11 |
| (d18:2/24:2)* | Brown Sugar | | oil_wt | | | |
| | Freq | | | | | |
| stearoyl | Falafel in Pita | −0.03796 | 5-9% White | 0.036949 | 0.303065 | 1.51E−11 |
| sphingomyelin | Bread Freq | | Cheese, | | | |
| (d18:1/18:0) | | | Cottage Freq | | | |
| N-methyltaurine | Egg, Hard | −0.08128 | Red Pepper | 0.053969 | 0.302497 | 1.65E−11 |
| | Boiled or Soft | | Freq | | | |
| | Freq | | | | | |
| lysine | Chicken or | 0.045408 | SF_Olives_wt | −0.0418 | 0.302457 | 1.66E−11 |
| | Turkey With | | | | | |
| | Skin Freq | | | | | |
| X - 17340 | SF_WhiteWheat_g_wt | 0.073187 | Fresh | 0.038033 | 0.300823 | 2.16E−11 |
| | | | Vegetable | | | |
| | | | Salad With | | | |
| | | | Dressing or | | | |
| | | | Oil Freq | | | |
| X - 13703 | SF_Yellow | 0.046598 | SF_Wholemeal | 0.041295 | 0.300461 | 2.29E−11 |
| | Cheese_wt | | Crackers_wt | | | |
| X - 24706 | SF_Brown | 0.015152 | SF_Tofu_wt | 0.013519 | 0.298699 | 3.03E−11 |
| | Rice_wt | | | | | |
| X - 22716 | SF_Cake_wt | −0.0602 | SF_Chocolate_wt | 0.058223 | 0.298683 | 3.04E−11 |
| X - 14082 | SF_Salmon_wt | −0.0267 | SF_Cappuccino_wt | 0.026149 | 0.298477 | 3.14E−11 |
| 4-allylphenol | SF_Rice_wt | −0.03687 | Lettuce Freq | 0.035869 | 0.298175 | 3.29E−11 |
| sulfate | | | | | | |
| 1-oleoyl-2- | SF_Yellow | −0.03448 | SF_Apple_wt | 0.031487 | 0.297782 | 3.50E−11 |
| docosahexaenoyl- | Cheese_wt | | | | | |
| GPC (18:1/22:6)* | | | | | | |
| X - 17354 | SF_Potatoes_wt | −0.03035 | SF_WhiteWheat_g_wt | −0.02666 | 0.296334 | 4.40E−11 |
| 6-oxopiperidine- | Ordinary | −0.03672 | SF_Omelette_wt | 0.036708 | 0.296239 | 4.46E−11 |
| 2-carboxylate | Bread or | | | | | |
| | Challah Freq | | | | | |
| X - 18240 | SF_Yellow | 0.040042 | Salty Snacks | −0.03397 | 0.296175 | 4.51E−11 |
| | Cheese_wt | | Freq | | | |
| theanine | SF_Tea_wt | 0.072302 | SF_Coffee_wt | −0.05017 | 0.296096 | 4.56E−11 |
| X - 24760 | Coffee Freq | 0.055548 | Thousand | −0.05301 | 0.296008 | 4.63E−11 |
| | | | Island | | | |
| | | | Dressing, | | | |
| | | | Garlic | | | |
| | | | Dressing Freq | | | |
| beta-hydroxyiso- | Beef, Veal, | 0.047254 | SF_Sugar Free | −0.04141 | 0.295258 | 5.20E−11 |
| valerate | Lamb, Pork, | | Gum_wt | | | |
| | Steak, Golash | | | | | |
| | Freq | | | | | |
| dodecenedioate | Salty Snacks | −0.06009 | SF_Wholemeal | 0.058701 | 0.293925 | 6.41E−11 |
| (C12:1-DC)* | Freq | | Bread_wt | | | |
| X - 11478 | Coated or | 0.056785 | Apricot Fresh | −0.05304 | 0.293803 | 6.53E−11 |
| | Stuffed | | or Dry, or | | | |
| | Cookies, | | Loquat Freq | | | |
| | Waffles or | | | | | |
| | Biscuits Freq | | | | | |
| X - 24736 | SF_Vegetable | 0.070155 | 5-9% White | −0.06725 | 0.293213 | 7.15E−11 |
| | Salad_wt | | Cheese, | | | |
| | | | Cottage Freq | | | |
| lactose | Sweet Dry | −0.06695 | SF_Carrots_wt | −0.06585 | 0.292312 | 8.22E−11 |
| | Wine, | | | | | |
| | Cocktails Freq | | | | | |
| 2-hydroxyoctanoate | 0-1.5% | −0.05124 | SF_Tomatoes_wt | −0.0447 | 0.291151 | 9.84E−11 |
| | Natural | | | | | |
| | Yogurt Freq | | | | | |
| trans-4- | Turkey | 0.039291 | Sausages | 0.037699 | 0.290927 | 1.02E−10 |
| hydroxyproline | Meatballs, | | such as | | | |
| | Beef, Chicken | | Salami Freq | | | |
| | Freq | | | | | |
| X - 17351 | Turkey | −0.04116 | SF_Kohlrabi_wt | 0.037389 | 0.290839 | 1.03E−10 |
| | Meatballs, | | | | | |
| | Beef, Chicken | | | | | |
| | Freq | | | | | |
| 1-methylnicotin- | Cooked | −0.04225 | Mandarin or | 0.038825 | 0.290374 | 1.11E−10 |
| amide | Legumes Freq | | Clementine | | | |
| | | | Freq | | | |
| acetoacetate | SF_Olives_wt | 0.051623 | SF_WholeWheat_g_wt | −0.04807 | 0.290273 | 1.13E−10 |
| X - 23782 | Simple | −0.04588 | Vegetable | 0.041358 | 0.290001 | 1.17E−10 |
| | Cookies or | | Soup Freq | | | |
| | Biscuits Freq | | | | | |
| X - 12818 | Pear Fresh, | 0.07774 | SF_WholeWheat_g_wt | 0.062202 | 0.288993 | 1.37E−10 |
| | Cooked or | | | | | |
| | Canned Freq | | | | | |
| 10- nonadecenoate | Simple | −0.0351 | SF_Yellow | 0.031683 | 0.288467 | 1.48E−10 |
| (19:1n9) | Cookies or | | Cheese_wt | | | |
| | Biscuits Freq | | | | | |
| X - 14314 | Coffee Freq | 0.02945 | SF_Cooked | −0.02384 | 0.287334 | 1.76E−10 |
| | | | Pumpkin_wt | | | |
| X - 24544 | Egg Recipes | 0.037427 | Alcoholic | 0.033114 | 0.287082 | 1.83E−10 |
| | Freq | | Drinks Freq | | | |
| gamma-glutamyl- | SF_Sugar Free | −0.03903 | Green Pepper | 0.035201 | 0.286693 | 1.94E−10 |
| leucine | Gum_wt | | Freq | | | |
| glutaryl- | Cooked | −0.04474 | Turkey | 0.025266 | 0.286566 | 1.98E−10 |
| carnitine (C5-DC) | Cereal such as | | Meatballs, | | | |
| | Oatmeal | | Beef, Chicken | | | |
| | Porridge Freq | | Freq | | | |
| hydantoin-5- | White or | −0.04693 | SF_Wine_wt | −0.0362 | 0.286558 | 1.98E−10 |
| propionic acid | Brown Sugar | | | | | |
| | Freq | | | | | |
| X - 12543 | Coffee Freq | 0.055644 | SF_WholeWheat_g_wt | 0.053565 | 0.284627 | 2.65E−10 |
| X - 17337 | Beer Freq | 0.051865 | SF_Tahini_wt | 0.051393 | 0.283435 | 3.16E−10 |
| dodecanedioate | SF_Coffee_wt | 0.056886 | SF_Butter_wt | 0.056491 | 0.283101 | 3.33E−10 |
| androstenediol | Shish Kebab | 0.047282 | Canned Tuna | 0.047157 | 0.283069 | 3.34E−10 |
| (3beta,17beta) | in Pita Bread | | or Tuna Salad | | | |
| monosulfate (1) | Freq | | Freq | | | |
| adipoylcarnitine | Fries Freq | 0.042836 | Beef, Veal, | 0.042155 | 0.282545 | 3.61E−10 |
| (C6-DC) | | | Lamb, Pork, | | | |
| | | | Steak, Golash | | | |
| | | | Freq | | | |
| pristanate | SF_Yellow | 0.057613 | SF_Butter_wt | 0.047975 | 0.282078 | 3.87E−10 |
| | Cheese_wt | | | | | |
| sphingomyelin | Beer Freq | −0.0458 | Pita Freq | −0.04203 | 0.280476 | 4.91E−10 |
| (d18:2/23:0, | | | | | | |
| d18:1/23:1, | | | | | | |
| d17:1/24:1)* | | | | | | |
| X - 24542 | Coffee Freq | 0.051751 | SF_Wholemeal | 0.04465 | 0.279864 | 5.37E−10 |
| | | | Bread_wt | | | |
| X - 22475 | SF_Tahini_wt | −0.05405 | Yeast Cakes | 0.041372 | 0.278663 | 6.40E−10 |
| | | | and Cookies | | | |
| | | | as Rogallach, | | | |
| | | | Croissant or | | | |
| | | | Donut Freq | | | |
| alpha-hydroxyiso- | Herbal Tea | −0.04036 | Sausages Freq | 0.031039 | 0.278512 | 6.55E−10 |
| valerate | Freq | | | | | |
| myristoylcarnitine | Couscous, | −0.0438 | SF_Butter_wt | 0.037691 | 0.278311 | 6.74E−10 |
| (C14) | Burgul, | | | | | |
| | Mamaliga, | | | | | |
| | Groats Freq | | | | | |
| X - 21411 | SF_Tomatoes_wt | −0.06179 | 0-1.5% | −0.04959 | 0.278127 | 6.93E−10 |
| | | | Natural | | | |
| | | | Yogurt Freq | | | |
| 1-(1-enyl- | SF_Tomatoes_wt | −0.03398 | Fish (not | 0.032764 | 0.277637 | 7.44E−10 |
| oleoyl)-GPE | | | Tuna) Pickled, | | | |
| (P-18:1)* | | | Dried, Smoked, | | | |
| | | | Canned Freq | | | |
| Fibrinopeptide | Cauliflower or | 0.015203 | SF_Rice_wt | −0.0111 | 0.277102 | 8.04E−10 |
| A (4-15)** | Broccoli Freq | | | | | |
| X - 11640 | Peanuts Freq | 0.047496 | Tahini Salad | 0.044656 | 0.276828 | 8.37E−10 |
| | | | Freq | | | |
| 2-hydroxy-3- | SF_WhiteWheat_g_wt | 0.034001 | Beef, Veal, | 0.030092 | 0.276598 | 8.65E−10 |
| methylvalerate | | | Lamb, Pork, | | | |
| | | | Steak, Golash | | | |
| | | | Freq | | | |
| dehydroiso- | Lemon Freq | −0.03975 | Canned Tuna | 0.037251 | 0.276405 | 8.90E−10 |
| androsterone | | | or Tuna Salad | | | |
| sulfate (DHEA-S) | | | Freq | | | |
| X - 12726 | Roll or | −0.05627 | 3% Milk Freq | −0.05352 | 0.276225 | 9.13E−10 |
| | Bageles Freq | | | | | |
| X - 13728 | SF_Chocolate_wt | 0.029507 | SF_Cottage | −0.02487 | 0.276 | 9.44E−10 |
| | | | cheese_wt | | | |
| cinnamoylglycine | SF_Water_wt | 0.041743 | SF_Dried | 0.0395 | 0.275153 | 1.07E−09 |
| | | | dates_wt | | | |
| X - 17685 | Coffee Freq | 0.061144 | SF_Soymilk_wt | 0.024588 | 0.274701 | 1.14E−09 |
| X - 12101 | SF_Whipped | 0.026953 | SF_WhiteWheat_g_wt | −0.0269 | 0.274215 | 1.22E−09 |
| | cream_wt | | | | | |
| glycocholenate | Falafel in Pita | 0.04998 | 0-1.5% | −0.02057 | 0.273996 | 1.26E−09 |
| sulfate* | Bread Freq | | Natural | | | |
| | | | Yogurt Freq | | | |
| 4-hydroxyphenyl | SF_Coffee_wt | 0.034855 | 5-9% Yellow | 0.033834 | 0.273101 | 1.43E−09 |
| pyruvate | | | Cheese Freq | | | |
| 1-(1-enyl-palmitoyl)- | SF_WholeWheat_g_wt | −0.04336 | Alcoholic | 0.034819 | 0.271544 | 1.79E−09 |
| 2-oleoyl-GPC | | | Drinks Freq | | | |
| (P-16:0/18:1)* | | | | | | |
| picolinoylglycine | SF_Cottage | 0.043259 | SF_Sugar Free | −0.03989 | 0.271367 | 1.83E−09 |
| | cheese_wt | | Gum_wt | | | |
| isocitrate | SF_Butter_wt | −0.05339 | Zucchini or | 0.050786 | 0.270843 | 1.98E−09 |
| | | | Eggplant Freq | | | |
| X - 24243 | Beef, Veal, | 0.039141 | Tahini Salad | −0.03466 | 0.270696 | 2.02E−09 |
| | Lamb, Pork, | | Freq | | | |
| | Steak, Golash | | | | | |
| | Freq | | | | | |
| androstenediol | SF_Beer_wt | 0.032141 | Fries Freq | 0.028093 | 0.270439 | 2.09E−09 |
| (3beta,17beta) | | | | | | |
| disulfate (2) | | | | | | |
| X - 11261 | Coated or | 0.033748 | SF_Mayonnaise_wt | 0.032668 | 0.269962 | 2.24E−09 |
| | Stuffed | | | | | |
| | Cookies, | | | | | |
| | Waffles or | | | | | |
| | Biscuits Freq | | | | | |
| X - 22162 | SF_Natural | 0.049682 | Dried Fruits | 0.044638 | 0.269887 | 2.26E−09 |
| | Yogurt_wt | | Freq | | | |
| X - 11470 | SF_Lettuce | 0.042756 | Beer Freq | 0.03851 | 0.268709 | 2.67E−09 |
| | Salad_wt | | | | | |
| 2-methylbutyryl | Processed | −0.02757 | SF_Apple_wt | 0.025362 | 0.268206 | 2.86E−09 |
| carnitine (C5) | Meat Free | | | | | |
| | Products Freq | | | | | |
| X - 12798 | SF_Potatoes_wt | −0.03424 | Green Tea | −0.03234 | 0.267623 | 3.11E−09 |
| | | | Freq | | | |
| dimethyl sulfoxide | SF_Onion_wt | 0.036787 | SF_Rice_wt | 0.034199 | 0.267466 | 3.18E−09 |
| (DMSO) | | | | | | |
| 2-aminooctanoate | SF_Beer_wt | 0.043264 | Peanuts Freq | 0.040677 | 0.266761 | 3.51E−09 |
| pentadecanoate | Regular Sodas | −0.03722 | Simple | −0.03633 | 0.26669 | 3.54E−09 |
| (15:0) | with Sugar | | Cookies or | | | |
| | Freq | | Biscuits Freq | | | |
| 1,2-dilinoleoyl- | Simple | 0.053441 | Alcoholic | 0.04957 | 0.266101 | 3.84E−09 |
| GPC (18:2/18:2) | Cookies or | | Drinks Freq | | | |
| | Biscuits Freq | | | | | |
| X - 18921 | SF_Dark | −0.03524 | Processed | 0.034302 | 0.26597 | 3.91E−09 |
| | Chocolate_wt | | Meat Free | | | |
| | | | Products Freq | | | |
| 1,2,3-benzenetriol | SF_Soymilk_wt | 0.016992 | SF_Hummus | 0.014111 | 0.26592 | 3.94E−09 |
| sulfate (2) | | | Salad_wt | | | |
| nonadecanoate | Yeast Cakes | −0.0177 | Potatoes | −0.01756 | 0.265878 | 3.96E−09 |
| (19:0) | and Cookies | | Boiled, | | | |
| | as Rogallach, | | Baked, | | | |
| | Croissant or | | Mashed, | | | |
| | Donut Freq | | Potatoes | | | |
| | | | Salad Freq | | | |
| gentisic acid- | Popsicle | −0.03124 | Cheese Cakes | −0.0283 | 0.265548 | 4.15E−09 |
| 5-glucoside | Without Dairy | | or Cream | | | |
| | Freq | | Cakes Freq | | | |
| X - 18606 | SF_Vegetable | 0.056105 | SF_Avocado_wt | 0.048188 | 0.26523 | 4.34E−09 |
| | Salad_wt | | | | | |
| hydroxy-N6,N6,N6- | Cooked | −0.04566 | SF_Tomatoes_wt | −0.03842 | 0.264635 | 4.71E−09 |
| trimethyllysine* | Cereal such as | | | | | |
| | Oatmeal | | | | | |
| | Porridge Freq | | | | | |
| 3-(3-hydroxy- | Mango Freq | −0.05152 | SF_Potatoes_wt | −0.04955 | 0.264207 | 5.00E−09 |
| phenyl)propionate | | | | | | |
| sulfate | | | | | | |
| cytosine | SF_Peanuts_wt | −0.03469 | SF_Rice | 0.032333 | 0.263535 | 5.48E−09 |
| | | | crackers_wt | | | |
| 2-hydroxynervonate* | Olives Freq | 0.043652 | Sugar | −0.03948 | 0.260982 | 7.77E−09 |
| | | | Sweetened | | | |
| | | | Chocolate | | | |
| | | | Milk Freq | | | |
| 1-(1-enyl-stearoyl)- | SF_Potatoes_wt | −0.0568 | Beef, Veal, | 0.056067 | 0.259863 | 9.05E−09 |
| 2-linoleoyl-GPE | | | Lamb, Pork, | | | |
| (P-18:0/18:2)* | | | Steak, Golash | | | |
| | | | Freq | | | |
| 1-palmitoyl-2- | SF_Smoked | 0.028026 | SF_Salmon_wt | 0.026445 | 0.259077 | 1.01E−08 |
| docosahexaenoyl-GPE | Salmon_wt | | | | | |
| (16:0/22:6)* | | | | | | |
| ADSGEGDFXAE | 5-9% White | 0.07299 | Artificial | 0.049585 | 0.259072 | 1.01E−08 |
| GGGVR* | Cheese, | | Sweeteners | | | |
| | Cottage Freq | | Freq | | | |
| 3-(3-hydroxyphenyl) | Regular Tea | −0.03674 | SF_Banana_wt | −0.03626 | 0.258787 | 1.05E−08 |
| propionate | Freq | | | | | |
| N-stearoyltaurine | SF_Carrots_wt | −0.03829 | SF_Butter_wt | 0.035164 | 0.258464 | 1.09E−08 |
| 4-vinylphenol | SF_Tahini_wt | 0.052734 | SF_Wine_wt | 0.035526 | 0.258277 | 1.12E−08 |
| sulfate | | | | | | |
| N-acetyltaurine | Yeast Cakes | 0.032979 | Beer Freq | 0.032928 | 0.258121 | 1.14E−08 |
| | and Cookies | | | | | |
| | as Rogallach, | | | | | |
| | Croissant or | | | | | |
| | Donut Freq | | | | | |
| X - 24293 | SF_Beer_wt | 0.070061 | SF_Water_wt | −0.01923 | 0.257891 | 1.18E−08 |
| tartronate | Fish (not | −0.03298 | SF_White | −0.02409 | 0.257375 | 1.27E−08 |
| (hydroxymalonate) | Tuna) Pickled, | | Cheese_wt | | | |
| | Dried, Smoked, | | | | | |
| | Canned Freq | | | | | |
| X - 22143 | SF_Vinaigrette_wt | −0.03471 | SF_WhiteWheat_g_wt | 0.034056 | 0.256825 | 1.36E−08 |
| pyrraline | SF_WhiteWheat_g_wt | 0.041669 | Garlic Freq | −0.03848 | 0.256457 | 1.43E−08 |
| 5-oxoproline | SF_Tomatoes_wt | −0.02449 | Granola or | 0.018581 | 0.256075 | 1.51E−08 |
| | | | Bernflaks | | | |
| | | | Freq | | | |
| margarate (17:0) | Olives Freq | 0.020651 | Butter Freq | 0.019122 | 0.255334 | 1.66E−08 |
| aconitate [cis | SF_WhiteWheat_g_wt | −0.04403 | SF_Lettuce_wt | −0.04111 | 0.254946 | 1.75E−08 |
| or trans] | | | | | | |
| 3,7-dimethylurate | Coffee Freq | 0.046929 | SF_Chocolate | 0.043673 | 0.253519 | 2.11E−08 |
| | | | wt | | | |
| 1-stearoyl-2- | SF_Salmon_wt | 0.032658 | SF_Onion_wt | −0.0273 | 0.252798 | 2.32E−08 |
| docosahexaenoyl-GPE | | | | | | |
| (18:0/22:6)* | | | | | | |
| X - 24801 | SF_WhiteWheat_g_wt | 0.039468 | SF_Vegetable | 0.035047 | 0.25267 | 2.36E−08 |
| | | | Salad_wt | | | |
| chiro-inositol | SF_Tomatoes_wt | 0.043455 | SF_Vegetable | 0.028689 | 0.251939 | 2.60E−08 |
| | | | Salad_wt | | | |
| trimethylamine | White or | −0.05219 | SF_Salmon_wt | 0.048754 | 0.251836 | 2.63E−08 |
| N-oxide | Brown Sugar | | | | | |
| | Freq | | | | | |
| 3-phenylpropionate | SF_Tahini_wt | 0.046797 | Coffee Freq | 0.035535 | 0.251731 | 2.67E−08 |
| (hydrocinnamate) | | | | | | |
| X - 12283 | Zucchini or | 0.05001 | SF_Kohlrabi_wt | 0.048317 | 0.251451 | 2.77E−08 |
| | Eggplant Freq | | | | | |
| X - 21410 | SF_Butter_wt | 0.102359 | SF_WholeWheat_g_wt | −0.09586 | 0.251284 | 2.83E−08 |
| vanillyl- | SF_Almonds_wt | 0.054517 | 5-9% White | 0.053657 | 0.250452 | 3.16E−08 |
| mandelate (VMA) | | | Cheese, | | | |
| | | | Cottage Freq | | | |
| N-acetylglycine | SF_Tomatoes_wt | −0.04563 | 5-9% Yellow | −0.04523 | 0.250257 | 3.24E−08 |
| | | | Cheese Freq | | | |
| X - 12812 | Apple Freq | 0.051147 | SF_Water_wt | 0.045874 | 0.250133 | 3.29E−08 |
| glycohyocholate | Regular Tea | −0.04174 | Cooked | 0.034447 | 0.24914 | 3.74E−08 |
| | Freq | | Legumes Freq | | | |
| palmitoyl | SF_Tahini_wt | 0.045093 | Lettuce Freq | 0.032665 | 0.248572 | 4.03E−08 |
| dihydrosphingo- | | | | | | |
| myelin | | | | | | |
| (d18:0/16:0)* | | | | | | |
| gamma-CEHC | Lemon Freq | −0.03876 | Simple | 0.033175 | 0.248418 | 4.11E−08 |
| | | | Cookies or | | | |
| | | | Biscuits Freq | | | |
| X - 12472 | Fried Fish | 0.051858 | SF_Yellow | 0.044072 | 0.247897 | 4.39E−08 |
| | Freq | | Cheese_wt | | | |
| 4-hydroxychloro- | Carrots, Fresh | 0.042961 | SF_Natural | 0.041852 | 0.247819 | 4.44E−08 |
| thalonil | or Cooked, | | Yogurt_wt | | | |
| | Carrot Juice | | | | | |
| | Freq | | | | | |
| 10-heptadecenoate | Butter Freq | 0.02122 | Apricot Fresh | 0.018248 | 0.247254 | 4.77E−08 |
| (17:1n7) | | | or Dry, or | | | |
| | | | Loquat Freq | | | |
| X - 23644 | Cooked | 0.033389 | SF_Tahini_wt | 0.031206 | 0.246648 | 5.16E−08 |
| | Legumes Freq | | | | | |
| X - 21821 | SF_Kohlrabi_wt | 0.035144 | Wholemeal or | 0.032936 | 0.246008 | 5.60E−08 |
| | | | Rye Bread | | | |
| | | | Freq | | | |
| X - 11444 | SF_Sugar Free | −0.04453 | Hummus | 0.04443 | 0.24496 | 6.40E−08 |
| | Gum_wt | | Salad Freq | | | |
| docosahexaenoyl- | Apricot Fresh | 0.036055 | Canned Tuna | 0.034324 | 0.244758 | 6.56E−08 |
| choline | or Dry, or | | or Tuna Salad | | | |
| | Loquat Freq | | Freq | | | |
| gamma-glutamyl- | Vegetable | −0.03889 | Hummus | 0.036507 | 0.244708 | 6.61E−08 |
| glutamine | Soup Freq | | Salad Freq | | | |
| valine | Cooked | −0.04865 | SF_Egg_wt | 0.04686 | 0.244693 | 6.62E−08 |
| | Cereal such as | | | | | |
| | Oatmeal | | | | | |
| | Porridge Freq | | | | | |
| X - 13723 | SF_Wholemeal | 0.034737 | Green Pepper | 0.030349 | 0.243279 | 7.92E−08 |
| | Bread_wt | | Freq | | | |
| indolepropionate | Beef, Veal, | −0.04225 | Beef or | −0.04146 | 0.242867 | 8.34E−08 |
| | Lamb, Pork, | | Chicken Soup | | | |
| | Steak, Golash | | Freq | | | |
| | Freq | | | | | |
| arabitol/xylitol | Mandarin or | 0.030668 | SF_WholeWheat_g_wt | 0.025042 | 0.24261 | 8.61E−08 |
| | Clementine | | | | | |
| | Freq | | | | | |
| carnitine | Green Tea | 0.044347 | SF_Sugar Free | −0.03366 | 0.241914 | 9.40E−08 |
| | Freq | | Gum_wt | | | |
| benzoylcarnitine* | Cooked | −0.05612 | SF_Banana_wt | −0.04617 | 0.24114 | 1.04E−07 |
| | Tomatoes, | | | | | |
| | Tomato | | | | | |
| | Sauce, | | | | | |
| | Tomato Soup | | | | | |
| | Freq | | | | | |
| X - 13729 | SF_Cucumber_wt | −0.05566 | 5-9% White | 0.054652 | 0.241117 | 1.04E−07 |
| | | | Cheese, | | | |
| | | | Cottage Freq | | | |
| X - 12739 | SF_Wholemeal | −0.0529 | Fried Fish | 0.049353 | 0.240909 | 1.07E−07 |
| | Bread_wt | | Freq | | | |
| 9-hydroxystearate | Hummus | −0.04738 | SF_Cucumber_wt | −0.04334 | 0.240733 | 1.09E−07 |
| | Salad Freq | | | | | |
| X - 21851 | SF_Peas_wt | 0.03663 | SF_Sugar_wt | 0.036351 | 0.239542 | 1.26E−07 |
| 13-methylmyristate | Butter Freq | 0.066217 | Sweet Dry | −0.05784 | 0.239418 | 1.28E−07 |
| | | | Wine, | | | |
| | | | Cocktails Freq | | | |
| 7-ethylguanine | SF_Apple_wt | −0.0402 | Beer Freq | 0.034817 | 0.238455 | 1.45E−07 |
| margaroylcarnitine* | Beef, Veal, | 0.038457 | SF_Heavy | 0.031674 | 0.238355 | 1.46E−07 |
| | Lamb, Pork, | | cream_wt | | | |
| | Steak, Golash | | | | | |
| | Freq | | | | | |
| docosapentaenoate | Mandarin or | 0.030573 | SF_Tahini_wt | −0.02998 | 0.238166 | 1.50E−07 |
| (n3 DPA; 22:5n3) | Clementine | | | | | |
| | Freq | | | | | |
| X - 24546 | SF_WholeWheat_g_wt | −0.06035 | Fries Freq | 0.0458 | 0.237124 | 1.70E−07 |
| X - 11787 | Fried Fish | 0.03232 | SF_Egg_wt | 0.028669 | 0.237089 | 1.71E−07 |
| | Freq | | | | | |
| X - 24527 | Fried Fish | 0.051738 | Schnitzel | −0.04649 | 0.236778 | 1.78E−07 |
| | Freq | | Turkey or | | | |
| | | | Chicken Freq | | | |
| 4-acetylphenol | SF_Cucumber_wt | 0.041261 | SF_Cereals_wt | 0.039656 | 0.235938 | 1.97E−07 |
| sulfate | | | | | | |
| sphingomyelin | Hummus | −0.03272 | Wholemeal or | 0.025919 | 0.235772 | 2.01E−07 |
| (d18:2/24:1, | Salad Freq | | Rye Bread | | | |
| d18:1/24:2)* | | | Freq | | | |
| cys-gly, oxidized | SF_Beef_wt | 0.029274 | SF_WhiteWheat_g_wt | 0.02397 | 0.235329 | 2.12E−07 |
| isoleucine | SF_Omelette_wt | 0.031634 | SF_Carrots_wt | −0.03141 | 0.23425 | 2.42E−07 |
| cysteinylglycine | SF_Beef_wt | 0.047552 | SF_WhiteWheat_g_wt | 0.045782 | 0.234121 | 2.46E−07 |
| disulfide* | | | | | | |
| 1-myristoyl-2- | Tahini Salad | −0.0497 | Wholemeal | 0.04605 | 0.234075 | 2.47E−07 |
| arachidonoyl-GPC | Freq | | Crackers Freq | | | |
| (14:0/20:4)* | | | | | | |
| 1-myristoyl- | SF_Coffee_wt | 0.052861 | Artificial | 0.051773 | 0.233586 | 2.62E−07 |
| glycerol (14:0) | | | Sweeteners | | | |
| | | | Freq | | | |
| alpha-ketoglutarate | SF_Omelette_wt | 0.035189 | SF_Cooked | −0.02958 | 0.233302 | 2.71E−07 |
| | | | beets_wt | | | |
| X - 24748 | Tahini Salad | 0.045675 | Sweet Dry | 0.03442 | 0.232703 | 2.92E−07 |
| | Freq | | Wine, | | | |
| | | | Cocktails Freq | | | |
| eicosanodioate | SF_Vegetable | 0.03333 | SF_Apple_wt | −0.03209 | 0.232681 | 2.92E−07 |
| | Salad_wt | | | | | |
| X - 24556 | Pita Freq | −0.05578 | SF_Beef_wt | 0.050743 | 0.232389 | 3.03E−07 |
| X - 23680 | SF_Tilapia_wt | −0.02589 | Cucumber | −0.02351 | 0.231176 | 3.50E−07 |
| | | | Freq | | | |
| acetylcarnitine | Nuts, | 0.042983 | Light Bread | −0.03972 | 0.231018 | 3.57E−07 |
| (C2) | almonds, | | Freq | | | |
| | pistachios | | | | | |
| | Freq | | | | | |
| hexanoylglutamine | SF_Cereals_wt | −0.03829 | 1% Milk Freq | −0.03708 | 0.230275 | 3.90E−07 |
| sphingomyelin | Pita Freq | −0.03438 | Rice Freq | −0.03337 | 0.229759 | 4.15E−07 |
| (d18:1/18:1, | | | | | | |
| d18:2/18:0) | | | | | | |
| sphingomyelin | 3% Milk Freq | 0.041461 | SF_Tomatoes_wt | −0.03591 | 0.229461 | 4.29E−07 |
| (d18:1/20:0, | | | | | | |
| d16:1/22:0)* | | | | | | |
| X - 23974 | White or | −0.04637 | SF_WholeWheat_g_wt | −0.03761 | 0.229335 | 4.36E−07 |
| | Brown Sugar | | | | | |
| | Freq | | | | | |
| X - 12212 | SF_Hummus | 0.063667 | SF_Tahini_wt | 0.047155 | 0.228975 | 4.55E−07 |
| | Salad_wt | | | | | |
| myristoleate | Butter Freq | 0.030639 | Artificial | 0.026067 | 0.228876 | 4.60E−07 |
| (14:1n5) | | | Sweeteners | | | |
| | | | Freq | | | |
| X - 13846 | SF_Wholemeal | 0.027088 | White or | −0.02225 | 0.22827 | 4.94E−07 |
| | Bread_wt | | Brown Sugar | | | |
| | | | Freq | | | |
| X - 21657 | SF_Water_wt | −0.0489 | Fried Fish | 0.04514 | 0.227519 | 5.40E−07 |
| | | | Freq | | | |
| X - 24352 | Cooked | 0.034955 | SF_Wine_wt | 0.024234 | 0.227239 | 5.58E−07 |
| | Legumes Freq | | | | | |
| beta- | SF_Cottage | −0.03389 | SF_Cooked | −0.02842 | 0.226628 | 6.00E−07 |
| citrylglutamate | cheese_wt | | beets_wt | | | |
| gluconate | SF_Hummus | −0.03687 | Wholemeal or | 0.03011 | 0.225883 | 6.54E−07 |
| | Salad_wt | | Rye Bread | | | |
| | | | Freq | | | |
| lignoceroyl- | SF_Olive | 0.035509 | SF_Chicken | −0.03551 | 0.225696 | 6.69E−07 |
| carnitine (C24)* | oil_wt | | breast_wt | | | |
| X - 24831 | SF_Burekas_wt | 0.024968 | Wholemeal or | −0.02427 | 0.225544 | 6.81E−07 |
| | | | Rye Bread | | | |
| | | | Freq | | | |
| Fibrinopeptide | Salty Snacks | −0.02003 | Lettuce Freq | 0.017207 | 0.224888 | 7.35E−07 |
| A (2-15)** | Freq | | | | | |
| gamma-glutamyl- | SF_WhiteWheat_g_wt | 0.029247 | White or | −0.02917 | 0.224408 | 7.77E−07 |
| isoleucine* | | | Brown Sugar | | | |
| | | | Freq | | | |
| X - 12846 | SF_Onion_wt | 0.049906 | SF_Pita_wt | 0.048261 | 0.223263 | 8.87E−07 |
| S-allylcysteine | SF_Pita_wt | 0.042506 | SF_Onion_wt | 0.042127 | 0.223104 | 9.03E−07 |
| tartarate | SF_Wine_wt | 0.043476 | SF_Coffee_wt | 0.029139 | 0.222895 | 9.25E−07 |
| ceramide | SF_Coffee_wt | 0.042692 | Coated or | −0.03819 | 0.222682 | 9.48E−07 |
| (d18:2/24:1, | | | Stuffed | | | |
| d18:1/24:2)* | | | Cookies, | | | |
| | | | Waffles or | | | |
| | | | Biscuits Freq | | | |
| X - 12714 | SF_Bread_wt | −0.05319 | Peach, | 0.030081 | 0.222567 | 9.61E−07 |
| | | | Nectarine, | | | |
| | | | Plum Freq | | | |
| 1-stearoyl-2- | SF_Wholemeal | 0.023354 | SF_Meatballs_wt | −0.02324 | 0.222212 | 1.00E−06 |
| linoleoyl-GPI | Light | | | | | |
| (18:0/18:2) | Bread_wt | | | | | |
| 1-linoleoyl- | 5-9% Yellow | −0.03418 | Light Bread | −0.0282 | 0.222057 | 1.02E−06 |
| GPC (18:2) | Cheese Freq | | Freq | | | |
| gamma-glutamyl- | 5-9% Yellow | 0.031077 | SF_Fried | −0.02981 | 0.221981 | 1.03E−06 |
| tyrosine | Cheese Freq | | eggplant_wt | | | |
| N-acetyl- | SF_Tahini_wt | 0.020461 | SF_WholeWheat_g_wt | −0.0182 | 0.221341 | 1.11E−06 |
| isoputreanine* | | | | | | |
| hexanoyl- | 1% Milk Freq | −0.03642 | SF_Wine_wt | 0.027807 | 0.21931 | 1.39E−06 |
| carnitine (C6) | | | | | | |
| X - 16944 | Egg, Hard | −0.03398 | SF_WhiteWheat_g_wt | 0.032391 | 0.2191 | 1.43E−06 |
| | Boiled or Soft | | | | | |
| | Freq | | | | | |
| sucrose | SF_Milk_wt | −0.03044 | SF_Whipped | −0.02593 | 0.218142 | 1.59E−06 |
| | | | cream_wt | | | |
| formimino- | SF_Egg_wt | 0.036909 | 5-9% Yellow | 0.03563 | 0.217446 | 1.72E−06 |
| glutamate | | | Cheese Freq | | | |
| arachidoyl- | SF_Egg_wt | 0.068078 | SF_Vegetable | 0.064361 | 0.217162 | 1.77E−06 |
| carnitine (C20)* | | | Salad_wt | | | |
| ximenoyl-carnitine | Roll or | −0.02959 | Coffee Freq | 0.026603 | 0.216738 | 1.86E−06 |
| (C26:1)* | Bageles Freq | | | | | |
| hydroquinone | White or | −0.03713 | Wholemeal or | 0.026508 | 0.216453 | 1.92E−06 |
| sulfate | Brown Sugar | | Rye Bread | | | |
| | Freq | | Freq | | | |
| caprylate (8:0) | SF_Butter_wt | 0.057074 | SF_Chocolate_wt | 0.042709 | 0.216 | 2.02E−06 |
| 3-methylcytidine | SF_Dark | −0.06036 | SF_Tahini_wt | −0.04877 | 0.215928 | 2.04E−06 |
| | Chocolate_wt | | | | | |
| riboflavin | 0-1.5% | 0.046853 | Orange or | 0.045733 | 0.215777 | 2.07E−06 |
| (Vitamin B2) | Natural | | Grapefruit | | | |
| | Yogurt Freq | | Freq | | | |
| X - 14662 | SF_Water_wt | −0.05161 | Vegetable | −0.04097 | 0.215721 | 2.08E−06 |
| | | | Soup Freq | | | |
| Fibrinopeptide | 3% Milk Freq | 0.010231 | SF_Cottage | 0.005469 | 0.215719 | 2.08E−06 |
| A(5-16)* | | | cheese_wt | | | |
| X - 17335 | Olives Freq | 0.055813 | SF_Tahini_wt | 0.052579 | 0.215692 | 2.09E−06 |
| 3-hydroxy-3- | White or | −0.0366 | Mayonnaise | −0.03014 | 0.214105 | 2.49E−06 |
| methylglutarate | Brown Sugar | | Including | | | |
| | Freq | | Light Freq | | | |
| N-palmitoyl- | Artificial | 0.073831 | Pear Fresh, | −0.06499 | 0.213887 | 2.55E−06 |
| heptadeca- | Sweeteners | | Cooked or | | | |
| sphingosine | Freq | | Canned Freq | | | |
| (d17:1/16:0)* | | | | | | |
| methyl-4- | SF_Omelette_wt | −0.0289 | SF_Coffee_wt | 0.027893 | 0.21387 | 2.56E−06 |
| hydroxybenzoate | | | | | | |
| sulfate | | | | | | |
| N-acetyl- | SF_Olive | −0.03452 | SF_WhiteWheat_g_wt | 0.031265 | 0.213562 | 2.65E−06 |
| cadaverine | oil_wt | | | | | |
| kynurenine | SF_Vegetable | 0.034849 | Apple Freq | 0.03172 | 0.212924 | 2.84E−06 |
| | Salad_wt | | | | | |
| 5alpha-androstan- | SF_WhiteWheat_g_wt | 0.044035 | SF_Coffee_wt | −0.04269 | 0.212505 | 2.97E−06 |
| 3alpha,17beta-diol | | | | | | |
| monosulfate (1) | | | | | | |
| X - 21807 | SF_Tea_wt | 0.045968 | SF_WhiteWheat_g_wt | −0.04431 | 0.211198 | 3.43E−06 |
| X - 16946 | Yeast Cakes | 0.041822 | Mandarin or | −0.0417 | 0.210387 | 3.74E−06 |
| | and Cookies | | Clementine | | | |
| | as Rogallach, | | Freq | | | |
| | Croissant or | | | | | |
| | Donut Freq | | | | | |
| X - 11485 | Red Pepper | 0.052431 | Light Bread | −0.05116 | 0.210384 | 3.75E−06 |
| | Freq | | Freq | | | |
| methionine | Avocado Freq | 0.037449 | Pasta or | −0.03732 | 0.210184 | 3.83E−06 |
| sulfone | | | Flakes Freq | | | |
| 3-methoxycatechol | SF_Soymilk_wt | 0.015205 | SF_Hummus | 0.011009 | 0.209983 | 3.91E−06 |
| sulfate (1) | | | Salad_wt | | | |
| N1-methyladenosine | Olives Freq | 0.033459 | Cooked | −0.02976 | 0.209787 | 4.00E−06 |
| | | | Legumes Freq | | | |
| andro steroid | SF_Rice | −0.05382 | Tahini Salad | −0.05136 | 0.209095 | 4.31E−06 |
| monosulfate | crackers_wt | | Freq | | | |
| C19H28O6S (1)* | | | | | | |
| X - 12712 | SF_Wholemeal | 0.012731 | SF_Potatoes_wt | 0.012558 | 0.208113 | 4.79E−06 |
| | Bread_wt | | | | | |
| X - 21470 | Fries Freq | 0.035121 | 5-9% White | −0.03463 | 0.208048 | 4.82E−06 |
| | | | Cheese, | | | |
| | | | Cottage Freq | | | |
| 1-oleoyl-2- | SF_Diet | −0.03429 | Butter Freq | −0.03057 | 0.208 | 4.84E−06 |
| docosahexaenoyl- | Coke_wt | | | | | |
| GPE (18:1/22:6)* | | | | | | |
| gamma-CEHC | SF_Roll_wt | 0.030995 | Simple | 0.024969 | 0.207064 | 5.36E−06 |
| glucuronide* | | | Cookies or | | | |
| | | | Biscuits Freq | | | |
| glycocholate | SF_Olive | 0.031883 | Sausages Freq | −0.03058 | 0.207005 | 5.39E−06 |
| | oil_wt | | | | | |
| carboxyethyl-GABA | SF_Date | −0.0288 | Sausages Freq | −0.02853 | 0.206737 | 5.55E−06 |
| | honey_wt | | | | | |
| N2,N2-dimethyl- | Couscous, | −0.05847 | Falafel in Pita | 0.051005 | 0.206381 | 5.76E−06 |
| guanosine | Burgul, | | Bread Freq | | | |
| | Mamaliga, | | | | | |
| | Groats Freq | | | | | |
| X - 21310 | SF_Canned | −0.03237 | 3% Milk Freq | 0.029754 | 0.206366 | 5.77E−06 |
| | Tuna Fish_wt | | | | | |
| glycocheno- | Coffee Freq | −0.04221 | SF_Schnitzel_wt | −0.02378 | 0.206165 | 5.89E−06 |
| deoxycholate | | | | | | |
| sulfate | | | | | | |
| N-acetyl-2- | White or | −0.02893 | SF_Cereals_wt | −0.0287 | 0.204614 | 6.95E−06 |
| aminooctanoate* | Brown Sugar | | | | | |
| | Freq | | | | | |
| X - 24410 | SF_Olives_wt | 0.034043 | Internal | 0.030694 | 0.20429 | 7.19E−06 |
| | | | Organs Freq | | | |
| 1-linoleoyl-2- | Couscous, | 0.027012 | Chicken or | −0.02318 | 0.204266 | 7.21E−06 |
| linolenoyl-GPC | Burgul, | | Turkey | | | |
| (18:2/18:3)* | Mamaliga, | | Without Skin | | | |
| | Groats Freq | | Freq | | | |
| glycerophospho- | Roll or | −0.03703 | SF_Wholemeal | −0.0367 | 0.204048 | 7.37E−06 |
| ethanolamine | Bageles Freq | | Bread_wt | | | |
| X - 21792 | SF_Hummus | −0.05434 | Olives Freq | 0.046019 | 0.203958 | 7.44E−06 |
| | Salad_wt | | | | | |
| 5-hydroxymethyl- | 5-9% Yellow | 0.034617 | SF_Carrots_wt | −0.03409 | 0.203854 | 7.52E−06 |
| 2-furoic acid | Cheese Freq | | | | | |
| pipecolate | SF_Brown | 0.035863 | SF_Tomatoes_wt | 0.032794 | 0.203201 | 8.06E−06 |
| | Rice_wt | | | | | |
| linoleoyl- | SF_Vegetable | 0.019081 | Cooked | 0.016292 | 0.203137 | 8.11E−06 |
| linoleoyl-glycerol | Salad_wt | | Legumes Freq | | | |
| (18:2/18:2) [1]* | | | | | | |
| 3-hydroxy-2- | SF_Beef_wt | 0.039663 | SF_Wine_wt | 0.036226 | 0.202718 | 8.48E−06 |
| ethylpropionate | | | | | | |
| 6-hydroxyindole | SF_Tomatoes_wt | −0.04018 | SF_Natural | 0.032374 | 0.20208 | 9.06E−06 |
| sulfate | | | Yogurt_wt | | | |
| ectoine | Sausages Freq | 0.035185 | SF_Potatoes_wt | 0.030595 | 0.201936 | 9.20E−06 |
| 3-methyladipate | SF_Tahini_wt | −0.0571 | SF_Coffee_wt | 0.04709 | 0.201769 | 9.36E−06 |
| 3-hydroxyiso- | White or | −0.04669 | Mandarin or | 0.04492 | 0.201677 | 9.45E−06 |
| butyrate | Brown Sugar | | Clementine | | | |
| | Freq | | Freq | | | |
| 1-palmitoyl- | SF_Rice | 0.034712 | SF_Chocolate_wt | 0.03144 | 0.201646 | 9.48E−06 |
| GPE (16:0) | crackers_wt | | | | | |
| 1-palmitoyl-2- | Lettuce Freq | 0.026417 | Tahini Salad | −0.0251 | 0.201385 | 9.74E−06 |
| oleoyl-GPC | | | Freq | | | |
| (16:0/18:1) | | | | | | |
| laurate (12:0) | Regular Sodas | −0.03317 | SF_Chocolate_wt | 0.031227 | 0.201362 | 9.76E−06 |
| | with Sugar | | | | | |
| | Freq | | | | | |
| X - 21441 | SF_Apple_wt | −0.0393 | SF_Red | 0.038817 | 0.201256 | 9.87E−06 |
| | | | pepper_wt | | | |
| X - 15674 | SF_Potatoes_wt | −0.05877 | Artificial | 0.057227 | 0.201037 | 1.01E−05 |
| | | | Sweeteners | | | |
| | | | Freq | | | |
| X - 21258 | Dried Fruits | 0.023898 | SF_Lettuce_wt | 0.020678 | 0.20092 | 1.02E−05 |
| | Freq | | | | | |
| sulfate* | SF_Hummus | −0.02733 | SF_Tahini_wt | −0.02677 | 0.199886 | 1.14E−05 |
| | Salad_wt | | | | | |
| docosahexaenoyl- | SF_Schnitzel_wt | −0.03707 | Canned Tuna | 0.03318 | 0.199601 | 1.17E−05 |
| carnitine | | | or Tuna Salad | | | |
| (C22:6)* | | | Freq | | | |
| fumarate | Granola or | 0.028395 | >=16% Yellow | 0.018041 | 0.199402 | 1.20E−05 |
| | Bernflaks | | Cheese Freq | | | |
| | Freq | | | | | |
| propionylglycine | SF_Rice_wt | −0.03042 | Herbal Tea | 0.026809 | 0.199252 | 1.21E−05 |
| | | | Freq | | | |
| 1-ribosyl- | SF_Cooked | 0.025185 | SF_Vegetable | 0.025109 | 0.198938 | 1.25E−05 |
| imidazoleacetate* | cauliflower_wt | | Salad_wt | | | |
| 16a-hydroxy | Tahini Salad | −0.04126 | 5-9% White | −0.03793 | 0.198708 | 1.28E−05 |
| DHEA 3-sulfate | Freq | | Cheese, | | | |
| | | | Cottage Freq | | | |
| androstenediol | SF_Tahini_wt | −0.03316 | Fries Freq | 0.033153 | 0.198299 | 1.34E−05 |
| (3beta,17beta) | | | | | | |
| disulfate (1) | | | | | | |
| pantothenate | White or | −0.03044 | Lettuce Freq | 0.026934 | 0.198254 | 1.34E−05 |
| | Brown Sugar | | | | | |
| | Freq | | | | | |
| X - 15461 | SF_Cooked | −0.02838 | Schnitzel | 0.02601 | 0.198187 | 1.35E−05 |
| | Sweet | | Turkey or | | | |
| | potato_wt | | Chicken Freq | | | |
| linoleoylcholine* | Sugar | 0.03653 | Sweet Potato | 0.03109 | 0.197571 | 1.44E−05 |
| | Sweetened | | Freq | | | |
| | Chocolate | | | | | |
| | Milk Freq | | | | | |
| 1-linoleoyl- | SF_Rice | 0.03124 | SF_Coffee_wt | 0.028794 | 0.197503 | 1.45E−05 |
| GPE (18:2)* | crackers_wt | | | | | |
| nisinate | SF_Sushi_wt | 0.061065 | Fish Cooked, | 0.045237 | 0.197461 | 1.46E−05 |
| (24:6n3) | | | Baked or | | | |
| | | | Grilled Freq | | | |
| arachidate | SF_Raisins_wt | 0.037987 | SF_WhiteWheat_g_wt | −0.02837 | 0.197399 | 1.47E−05 |
| (20:0) | | | | | | |
| octadecenedioate | SF_Onion_wt | −0.03005 | SF_Wholemeal | 0.024611 | 0.196535 | 1.60E−05 |
| (C18:1-DC)* | | | Bread_wt | | | |
| 1,2-dilinoleoyl-GPE | SF_Tahini_wt | 0.043381 | SF_Banana_wt | −0.03691 | 0.19616 | 1.66E−05 |
| (18:2/18:2)* | | | | | | |
| acisoga | 1% Milk Freq | −0.02684 | Fries Freq | 0.023845 | 0.19604 | 1.68E−05 |
| propionylcarnitine | SF_WhiteWheat_g_wt | 0.043198 | Processed | −0.04207 | 0.195829 | 1.72E−05 |
| (C3) | | | Meat Free | | | |
| | | | Products Freq | | | |
| 1-linoleoyl-GPG | SF_Milk_wt | −0.03395 | Cooked | 0.033922 | 0.194581 | 1.95E−05 |
| (18:2)* | | | Legumes Freq | | | |
| X - 12263 | SF_Wholemeal | 0.043779 | Green Pepper | 0.04186 | 0.194366 | 1.99E−05 |
| | Bread_wt | | Freq | | | |
| X - 13553 | Cooked | −0.03527 | SF_Tomatoes_wt | −0.03475 | 0.194158 | 2.03E−05 |
| | Tomatoes, | | | | | |
| | Tomato | | | | | |
| | Sauce, | | | | | |
| | Tomato Soup | | | | | |
| | Freq | | | | | |
| 5-hydroxyindole | Apple Freq | 0.03995 | SF_Apple_wt | 0.03013 | 0.193696 | 2.13E−05 |
| acetate | | | | | | |
| X - 21295 | SF_WhiteWheat_g_wt | 0.038392 | Canned Tuna | 0.030332 | 0.1934 | 2.19E−05 |
| | | | or Tuna Salad | | | |
| | | | Freq | | | |
| Fibrinopeptide | SF_Meatballs_wt | 0.003791 | Processed | −0.00339 | 0.192825 | 2.32E−05 |
| A (3-16)** | | | Meat Free | | | |
| | | | Products Freq | | | |
| N-palmitoyl- | Coffee Freq | 0.030033 | Artificial | 0.02767 | 0.192811 | 2.33E−05 |
| sphingosine | | | Sweeteners | | | |
| (d18:1/16:0) | | | Freq | | | |
| X - 17677 | SF_Chicken | 0.04053 | Granola or | 0.029364 | 0.192639 | 2.37E−05 |
| | soup_wt | | Bernflaks | | | |
| | | | Freq | | | |
| 3-hydroxyhexanoate | Butter Freq | 0.035739 | SF_Yellow | 0.031216 | 0.191465 | 2.66E−05 |
| | | | Cheese_wt | | | |
| sphingomyelin | SF_Chocolate | −0.03649 | SF_Tahini_wt | −0.03128 | 0.190812 | 2.84E−05 |
| (d18:1/24:1, | spread_wt | | | | | |
| d18:2/24:0)* | | | | | | |
| 1-carboxyethyl- | Tahini Salad | −0.02825 | SF_White | 0.022235 | 0.190617 | 2.89E−05 |
| phenylalanine | Freq | | Cheese_wt | | | |
| 3-hydroxy- | SF_WhiteWheat_g_wt | −0.02869 | SF_WholeWheat_g_wt | −0.02762 | 0.190569 | 2.91E−05 |
| butyrate (BHBA) | | | | | | |
| X - 15469 | Artificial | −0.01778 | SF_Wine_wt | 0.014844 | 0.189997 | 3.07E−05 |
| | Sweeteners | | | | | |
| | Freq | | | | | |
| leucylglycine | 5-9% White | −0.03481 | Pastrami or | −0.02909 | 0.189282 | 3.30E−05 |
| | Cheese, | | Smoked Turkey | | | |
| | Cottage Freq | | Breast Freq | | | |
| X - 23587 | SF_Onion_wt | −0.03805 | SF_Milk_wt | −0.03623 | 0.189237 | 3.31E−05 |
| gamma-glutamyl- | SF_Tomatoes_wt | −0.01742 | 5-9% Yellow | 0.015729 | 0.188976 | 3.40E−05 |
| phenylalanine | | | Cheese Freq | | | |
| sphingomyelin | Fish Cooked, | 0.049286 | Hummus | −0.04367 | 0.188841 | 3.44E−05 |
| (d18:1/22:1, | Baked or | | Salad Freq | | | |
| d18:2/22:0, | Grilled Freq | | | | | |
| d16:1/24:1)* | | | | | | |
| X - 24849 | SF_Potatoes_wt | 0.030353 | SF_Tomatoes_wt | 0.029985 | 0.18881 | 3.45E−05 |
| 1-stearoyl-2- | Onion Freq | 0.020547 | Beef, Veal, | −0.01732 | 0.188755 | 3.47E−05 |
| arachidonoyl-GPE | | | Lamb, Pork, | | | |
| (18:0/20:4) | | | Steak, Golash | | | |
| | | | Freq | | | |
| 17alpha-hydroxy- | Fries Freq | 0.053104 | Sugar | 0.050486 | 0.188227 | 3.65E−05 |
| pregnenolone | | | Sweetened | | | |
| 3-sulfate | | | Chocolate | | | |
| | | | Milk Freq | | | |
| myo-inositol | Orange or | 0.033163 | SF_Hummus | −0.02811 | 0.188037 | 3.72E−05 |
| | Grapefruit | | Salad_wt | | | |
| | Freq | | | | | |
| 17alpha-hydroxy- | SF_White | −0.06329 | Fresh | −0.05937 | 0.187966 | 3.75E−05 |
| pregnanolone | Cheese_wt | | Vegetable | | | |
| glucuronide | | | Salad Without | | | |
| | | | Dressing or | | | |
| | | | Oil Freq | | | |
| arachidonoyl- | Cauliflower or | −0.02408 | SF_Burekas_wt | −0.01572 | 0.187792 | 3.81E−05 |
| carnitine | Broccoli Freq | | | | | |
| (C20:4) | | | | | | |
| stearidonate | White or | −0.03285 | Simple | −0.03197 | 0.187772 | 3.82E−05 |
| (18:4n3) | Brown Sugar | | Cookies or | | | |
| | Freq | | Biscuits Freq | | | |
| gamma-glutamyl- | >=16% Yellow | 0.029051 | Beef or | 0.027522 | 0.187561 | 3.90E−05 |
| alpha-lysine | Cheese Freq | | Chicken Soup | | | |
| | | | Freq | | | |
| 3-indoxyl sulfate | SF_Tomatoes_wt | −0.03727 | 3% Milk Freq | 0.035598 | 0.187501 | 3.92E−05 |
| 1-stearoyl-2- | SF_Onion_wt | −0.02766 | SF_Rice | 0.024402 | 0.187394 | 3.96E−05 |
| linoleoyl-GPC | | | crackers_wt | | | |
| (18:0/18:2)* | | | | | | |
| X - 17327 | 5-9% White | 0.045741 | Falafel in Pita | 0.045124 | 0.187095 | 4.07E−05 |
| | Cheese, | | Bread Freq | | | |
| | Cottage Freq | | | | | |
| 1-stearoyl-2- | SF_Chicken | −0.02485 | SF_Peach_wt | 0.024598 | 0.187092 | 4.08E−05 |
| oleoyl-GPC | breast_wt | | | | | |
| (18:0/18:1) | | | | | | |
| 1-stearoyl-GPC | SF_Meatballs_wt | −0.01497 | SF_Chocolate_wt | 0.013615 | 0.185969 | 4.54E−05 |
| (18:0) | | | | | | |
| X - 23593 | Parsley, | 0.02961 | Potatoes | −0.0296 | 0.18592 | 4.56E−05 |
| | Celery, | | Boiled, | | | |
| | Fennel, Dill, | | Baked, | | | |
| | Cilantro, | | Mashed, | | | |
| | Green Onion | | Potatoes | | | |
| | Freq | | Salad Freq | | | |
| 1-linoleoyl-GPI | Vegetable | 0.028036 | Sweet Potato | 0.024532 | 0.185749 | 4.64E−05 |
| (18:2)* | Soup Freq | | Freq | | | |
| linolenate | Juice Freq | −0.01804 | 1% Milk Freq | −0.0058 | 0.185322 | 4.83E−05 |
| [alpha or gamma; | | | | | | |
| (18:3n3 or 6)] | | | | | | |
| glucuronate | Coffee Freq | 0.041991 | White or | −0.04054 | 0.185166 | 4.90E−05 |
| | | | Brown Sugar | | | |
| | | | Freq | | | |
| cerotoylcarnitine | Roll or | −0.04024 | SF_Carrots_wt | −0.03469 | 0.184952 | 5.01E−05 |
| (C26)* | Bageles Freq | | | | | |
| alpha-tocopherol | Roll or | −0.03205 | White or | −0.01876 | 0.184772 | 5.09E−05 |
| | Bageles Freq | | Brown Sugar | | | |
| | | | Freq | | | |
| cystine | SF_Wholemeal | 0.034485 | SF_Potatoes_wt | 0.031358 | 0.184703 | 5.13E−05 |
| | Bread_wt | | | | | |
| vanillic alcohol | SF_Coffee_wt | 0.035805 | SF_Soymilk_wt | 0.029788 | 0.184282 | 5.34E−05 |
| sulfate | | | | | | |
| palmitoleate | SF_Tahini_wt | −0.01717 | SF_Red | 0.015473 | 0.18346 | 5.77E−05 |
| (16:1n7) | | | pepper_wt | | | |
| o-cresol sulfate | Regular Tea | −0.02633 | Butter Freq | 0.023206 | 0.182833 | 6.12E−05 |
| | Freq | | | | | |
| 1-palmitoyl-2- | Onion Freq | 0.041663 | Apple Freq | −0.04109 | 0.1823 | 6.44E−05 |
| arachidonoyl-GPC | | | | | | |
| (16:0/20:4n6) | | | | | | |
| methylsuccinoyl- | Peach, | 0.042892 | SF_Vegetable | 0.040529 | 0.18002 | 7.97E−05 |
| carnitine (1) | Nectarine, | | Salad_wt | | | |
| | Plum Freq | | | | | |
| X - 24972 | SF_Boiled | −0.02669 | Avocado Freq | −0.0243 | 0.179942 | 8.03E−05 |
| | corn_wt | | | | | |
| X - 23666 | Shish Kebab | 0.026392 | SF_Beef_wt | 0.021242 | 0.179797 | 8.13E−05 |
| | in Pita Bread | | | | | |
| | Freq | | | | | |
| decanoylcarnitine | Cornflakes | −0.02509 | Artificial | −0.02091 | 0.178951 | 8.80E−05 |
| (C10) | Freq | | Sweeteners | | | |
| | | | Freq | | | |
| X - 21353 | Tahini Salad | 0.040996 | Hummus | 0.032797 | 0.177955 | 9.64E−05 |
| | Freq | | Salad Freq | | | |
| etiocholanolone | SF_Wine_wt | 0.032826 | Artificial | −0.03212 | 0.177909 | 9.68E−05 |
| glucuronide | | | Sweeteners | | | |
| | | | Freq | | | |
| X - 17353 | SF_Lentils_wt | 0.016303 | SF_Falafel_wt | 0.015272 | 0.177635 | 9.93E−05 |
| X - 24329 | SF_WhiteWheat_g_wt | 0.022406 | Couscous, | −0.02069 | 0.177373 | 0.000102 |
| | | | Burgul, | | | |
| | | | Mamaliga, | | | |
| | | | Groats Freq | | | |
| 2-arachidonoyl- | Fresh | −0.04959 | Green Tea | −0.04484 | 0.177237 | 0.000103 |
| glycerol (20:4) | Vegetable | | Freq | | | |
| | Salad With | | | | | |
| | Dressing or | | | | | |
| | Oil Freq | | | | | |
| sarcosine | SF_Apple_wt | 0.036839 | SF_Vegetable | 0.036783 | 0.176765 | 0.000108 |
| | | | Salad_wt | | | |
| alpha-ketobutyrate | SF_Orange_wt | −0.03408 | SF_Cooked | −0.03106 | 0.176716 | 0.000108 |
| | | | Sweet | | | |
| | | | potato_wt | | | |
| citrate | SF_WhiteWheat_g_wt | −0.04212 | Schnitzel | −0.03685 | 0.176704 | 0.000108 |
| | | | Turkey or | | | |
| | | | Chicken Freq | | | |
| pregnenolone | Lettuce Freq | −0.0326 | SF_Pita_wt | 0.028233 | 0.17657 | 0.000109 |
| sulfate | | | | | | |
| eicosenoate | Regular Sodas | −0.02684 | Jachnun, | −0.01683 | 0.176179 | 0.000113 |
| (20:1) | with Sugar | | Mlawah, | | | |
| | Freq | | Kubana, | | | |
| | | | Cigars Freq | | | |
| 5alpha-androstan- | SF_Tahini_wt | −0.03626 | SF_Beef_wt | 0.033247 | 0.175586 | 0.00012 |
| 3beta,17beta-diol | | | | | | |
| monosulfate (2) | | | | | | |
| hypotaurine | SF_Cookies_wt | 0.028012 | 3% Milk Freq | −0.0267 | 0.175581 | 0.00012 |
| tauro-beta- | Peas, Green | 0.042853 | SF_Beef_wt | −0.0424 | 0.17546 | 0.000121 |
| muricholate | Beans or Okra | | | | | |
| | Cooked Freq | | | | | |
| eicosapentaenoyl- | SF_Dark | 0.051286 | Salty Cheese, | 0.040596 | 0.17522 | 0.000124 |
| choline | Chocolate_wt | | Tzfatit, | | | |
| | | | Bulgarian, | | | |
| | | | Brinza, Thick | | | |
| | | | Slice Freq | | | |
| 1-oleoyl-GPE | SF_Olive | 0.023964 | SF_Rice | 0.023958 | 0.174709 | 0.00013 |
| (18:1) | oil_wt | | crackers_wt | | | |
| 1-palmitoyl-2- | SF_Rice | 0.030801 | SF_Onion_wt | −0.02794 | 0.174458 | 0.000133 |
| arachidonoyl-GPE | crackers_wt | | | | | |
| (16:0/20:4)* | | | | | | |
| androsterone | SF_Pita_wt | 0.021753 | Lemon Freq | −0.02021 | 0.173636 | 0.000143 |
| sulfate | | | | | | |
| 2-acetamidophenol | SF_Natural | 0.05103 | Brussels | −0.03924 | 0.172249 | 0.000162 |
| sulfate | Yogurt_wt | | Sprouts, | | | |
| | | | Green or Red | | | |
| | | | Cabbage Freq | | | |
| X - 01911 | Sausages Freq | 0.04778 | Onion Freq | 0.046101 | 0.172198 | 0.000162 |
| nicotinamide | Pasta or | 0.022404 | SF_Cappuccino_wt | −0.02196 | 0.172061 | 0.000164 |
| | Flakes Freq | | | | | |
| X - 11522 | SF_Tomatoes_wt | −0.02166 | SF_Potatoes_wt | 0.020857 | 0.171532 | 0.000172 |
| X - 12753 | SF_Coleslaw_wt | 0.018072 | SF_Majadra_wt | 0.013166 | 0.171179 | 0.000178 |
| N-palmitoyl- | SF_Sugar Free | −0.05475 | SF_Carrots_wt | −0.04871 | 0.170927 | 0.000182 |
| sphinganine | Gum_wt | | | | | |
| (d18:0/16:0) | | | | | | |
| X - 12844 | SF_Parsley_wt | −0.03563 | SF_Hummus | 0.031822 | 0.170888 | 0.000182 |
| | | | Salad_wt | | | |
| X - 12410 | SF_Beet_wt | 0.026455 | SF_Coffee_wt | 0.023122 | 0.170403 | 0.00019 |
| erucate | Fish Cooked, | 0.027024 | SF_Butter_wt | 0.022244 | 0.169009 | 0.000215 |
| (22:1n9) | Baked or | | | | | |
| | Grilled Freq | | | | | |
| X - 16964 | SF_Tea_wt | −0.05898 | SF_White | −0.05305 | 0.168603 | 0.000223 |
| | | | Cheese_wt | | | |
| palmitoyl- | Couscous, | −0.03679 | Fried Fish | 0.033518 | 0.167495 | 0.000246 |
| carnitine (C16) | Burgul, | | Freq | | | |
| | Mamaliga, | | | | | |
| | Groats Freq | | | | | |
| glyco-beta- | Mayonnaise | −0.04312 | Artificial | −0.03835 | 0.167404 | 0.000248 |
| muricholate** | Including | | Sweeteners | | | |
| | Light Freq | | Freq | | | |
| X - 21628 | SF_Coke_wt | −0.01938 | Fries Freq | −0.01753 | 0.167041 | 0.000255 |
| gamma- | Cooked | 0.026177 | Cooked | 0.025754 | 0.166456 | 0.000269 |
| glutamylglycine | Vegetable | | Legumes Freq | | | |
| | Salads Freq | | | | | |
| kynurenate | SF_Omelette_wt | 0.033652 | Apple Freq | 0.033305 | 0.166433 | 0.000269 |
| proline | SF_Sugar Free | −0.03186 | Carrots, Fresh | −0.02655 | 0.166004 | 0.000279 |
| | Gum_wt | | or Cooked, | | | |
| | | | Carrot Juice | | | |
| | | | Freq | | | |
| X - 21285 | SF_Beer_wt | 0.044204 | Fries Freq | 0.036011 | 0.165774 | 0.000285 |
| 3-hydroxyoctanoate | SF_Tomatoes_wt | −0.03953 | SF_Carrots_wt | −0.03756 | 0.165517 | 0.000291 |
| N6,N6,N6- | SF_Yellow | −0.02767 | Chicken or | 0.022466 | 0.16522 | 0.000299 |
| trimethyllysine | Cheese_wt | | Turkey With | | | |
| | | | Skin Freq | | | |
| phenylacetate | SF_Apple_wt | 0.039486 | SF_Potatoes_wt | −0.03667 | 0.165022 | 0.000304 |
| glutamine | 0.5-3% White | −0.02333 | 1% Milk Freq | −0.02017 | 0.164013 | 0.000331 |
| | Cheese, | | | | | |
| | Cottage Freq | | | | | |
| homocitrulline | SF_Ice | 0.037791 | SF_Cereals_wt | 0.035405 | 0.163593 | 0.000343 |
| | cream_wt | | | | | |
| X - 21659 | SF_Avocado_wt | 0.054468 | SF_Pickled | 0.053846 | 0.163379 | 0.00035 |
| | | | cucumber_wt | | | |
| N-acetyltyrosine | Banana Freq | −0.03468 | SF_Watermelon_wt | 0.029821 | 0.16333 | 0.000351 |
| X - 21474 | Onion Freq | 0.056572 | SF_Avocado_wt | 0.054782 | 0.163136 | 0.000357 |
| X - 12026 | SF_Brown | −0.03309 | SF_Almonds_wt | 0.028343 | 0.163007 | 0.000361 |
| | Rice_wt | | | | | |
| xylose | SF_Pita_wt | −0.03725 | Shish Kebab | −0.03658 | 0.162979 | 0.000362 |
| | | | in Pita Bread | | | |
| | | | Freq | | | |
| dihomo-linolenoyl- | Sugar | 0.034004 | Peach, | 0.033119 | 0.162667 | 0.000371 |
| choline | Sweetened | | Nectarine, | | | |
| | Chocolate | | Plum Freq | | | |
| | Milk Freq | | | | | |
| X - 24106 | SF_Potatoes_wt | −0.03575 | Rice Freq | 0.027367 | 0.162598 | 0.000374 |
| X - 14095 | Processed | 0.023087 | SF_Wine_wt | 0.016406 | 0.162554 | 0.000375 |
| | Meat Free | | | | | |
| | Products Freq | | | | | |
| tyrosine | SF_Cappuccino_wt | 0.019225 | SF_Banana_wt | −0.01726 | 0.161408 | 0.000413 |
| dihomo-linoleoyl- | SF_WhiteWheat_g_wt | 0.043029 | Hummus | 0.042381 | 0.161336 | 0.000415 |
| carnitine | | | Salad Freq | | | |
| (C20:2)* | | | | | | |
| asparagine | SF_Apple_wt | 0.025085 | Fish Cooked, | −0.01962 | 0.161115 | 0.000423 |
| | | | Baked or | | | |
| | | | Grilled Freq | | | |
| N-acetylmethionine | >=16% Yellow | 0.002655 | Light Bread | −0.00164 | 0.160831 | 0.000433 |
| | Cheese Freq | | Freq | | | |
| X - 21364 | Egg Recipes | 0.029407 | SF_Pizza_wt | 0.028524 | 0.16065 | 0.00044 |
| | Freq | | | | | |
| X - 25116 | SF_Fried | −0.02065 | SF_Wholemeal | 0.020403 | 0.16014 | 0.000459 |
| | onions_wt | | Bread_wt | | | |
| 3beta- | SF_Red | 0.026768 | SF_Beer_wt | 0.026049 | 0.160076 | 0.000461 |
| hydroxy-5- | pepper_wt | | | | | |
| cholestenoate | | | | | | |
| dopamine 4- | SF_Bread_wt | −0.03894 | Pastrami or | −0.03467 | 0.159893 | 0.000469 |
| sulfate | | | Smoked Turkey | | | |
| | | | Breast Freq | | | |
| pyridoxate | SF_Cucumber_wt | 0.040288 | SF_Sugar Free | 0.038202 | 0.159679 | 0.000477 |
| | | | Gum_wt | | | |
| N-acetyl-1- | SF_Omelette_wt | 0.040531 | SF_Tahini_wt | −0.03737 | 0.159006 | 0.000504 |
| methylhistidine* | | | | | | |
| guanidinoacetate | Cooked | 0.038237 | SF_Salmon_wt | −0.03398 | 0.158661 | 0.000519 |
| | Legumes Freq | | | | | |
| 21-hydroxy- | Tahini Salad | −0.0262 | Lemon Freq | −0.02491 | 0.158595 | 0.000522 |
| pregnenolone | Freq | | | | | |
| disulfate | | | | | | |
| malate | SF_Roll_wt | −0.01369 | Canned Tuna | −0.01304 | 0.158508 | 0.000525 |
| | | | or Tuna Salad | | | |
| | | | Freq | | | |
| oleoylcarnitine | 1% Milk Freq | −0.01911 | Artificial | −0.01744 | 0.158465 | 0.000527 |
| (C18:1) | | | Sweeteners | | | |
| | | | Freq | | | |
| X - 12206 | SF_Vegetable | 0.033866 | White or | −0.03028 | 0.15836 | 0.000532 |
| | Salad_wt | | Brown Sugar | | | |
| | | | Freq | | | |
| X - 12063 | Tahini Salad | −0.02703 | Cheese Cakes | 0.017972 | 0.158256 | 0.000536 |
| | Freq | | or Cream | | | |
| | | | Cakes Freq | | | |
| oleoyl | 5-9% White | −0.02099 | Olives Freq | 0.020749 | 0.158101 | 0.000543 |
| ethanolamide | Cheese, | | | | | |
| | Cottage Freq | | | | | |
| glutamate | SF_Butter_wt | 0.012596 | SF_Sugar Free | −0.01167 | 0.15775 | 0.000559 |
| | | | Gum_wt | | | |
| phenylacetyl- | Onion Freq | −0.0358 | SF_Apple_wt | 0.030598 | 0.15732 | 0.000579 |
| glutamine | | | | | | |
| X - 12096 | SF_Sugar Free | −0.05257 | SF_Wholemeal | 0.042001 | 0.156575 | 0.000616 |
| | Gum_wt | | Bread_wt | | | |
| 1-linoleoyl- | Chicken or | −0.04173 | Pastrami or | −0.03293 | 0.155799 | 0.000656 |
| GPA (18:2)* | Turkey | | Smoked Turkey | | | |
| | Without Skin | | Breast Freq | | | |
| | Freq | | | | | |
| X - 23654 | SF_White | 0.03125 | SF_Potatoes_wt | 0.029627 | 0.155531 | 0.00067 |
| | Cheese_wt | | | | | |
| glycosyl-N- | Hummus | −0.04128 | Milk or Dark | 0.040666 | 0.155084 | 0.000695 |
| stearoyl- | Salad Freq | | Chocolate | | | |
| sphingosine | | | Freq | | | |
| (d18:1/18:0) | | | | | | |
| X - 12906 | Watermelon | 0.043948 | White or | −0.04039 | 0.154102 | 0.000752 |
| | Freq | | Brown Sugar | | | |
| | | | Freq | | | |
| 3-sulfo-L-alanine | SF_Milk_wt | 0.019177 | Lettuce Freq | −0.01653 | 0.153753 | 0.000773 |
| X - 24498 | 3% Milk Freq | −0.03907 | SF_Wine_wt | 0.034558 | 0.153726 | 0.000775 |
| phosphate | Pita Freq | −0.02025 | SF_Rice | −0.01902 | 0.153716 | 0.000776 |
| | | | crackers_wt | | | |
| S-carboxymethyl- | Mandarin or | −0.04016 | Milk or Dark | −0.03917 | 0.153566 | 0.000785 |
| L-cysteine | Clementine | | Chocolate | | | |
| | Freq | | Freq | | | |
| N-oleoyltaurine | Fresh | 0.042512 | Parsley, | 0.04102 | 0.151729 | 0.000909 |
| | Vegetable | | Celery, | | | |
| | Salad With | | Fennel, Dill, | | | |
| | Dressing or | | Cilantro, | | | |
| | Oil Freq | | Green Onion | | | |
| | | | Freq | | | |
| cysteinylglycine | Carrots, Fresh | −0.02377 | Wholemeal | −0.02257 | 0.150761 | 0.000981 |
| | or Cooked, | | Crackers Freq | | | |
| | Carrot Juice | | | | | |
| | Freq | | | | | |
| X - 24699 | SF_WhiteWheat_g_wt | 0.038762 | Couscous, | −0.03427 | 0.149932 | 0.001047 |
| | | | Burgul, | | | |
| | | | Mamaliga, | | | |
| | | | Groats Freq | | | |
| N6-succinyl- | SF_Rice | 0.024343 | SF_Hummus_wt | 0.024016 | 0.149726 | 0.001064 |
| adenosine | crackers_wt | | | | | |
| sphingomyelin | >=16% Yellow | 0.016074 | Fresh | 0.015044 | 0.149555 | 0.001078 |
| (d18:0/18:0, | Cheese Freq | | Vegetable | | | |
| d19:0/17:0)* | | | Salad Without | | | |
| | | | Dressing or | | | |
| | | | Oil Freq | | | |
| azelate | Orange or | −0.02521 | Regular Tea | −0.02091 | 0.149205 | 0.001108 |
| (nonanedioate) | Grapefruit | | Freq | | | |
| | Freq | | | | | |
| X - 24813 | SF_Fried | −0.03042 | Egg Recipes | 0.028449 | 0.149165 | 0.001111 |
| | eggplant_wt | | Freq | | | |
| gamma-glutamyl-2- | Green Tea | 0.02143 | SF_WhiteWheat_g_wt | −0.02096 | 0.148814 | 0.001142 |
| aminobutyrate | Freq | | | | | |
| 2-docosahexaenoyl- | SF_Pear_wt | −0.0529 | SF_Tahini_wt | −0.04006 | 0.148349 | 0.001184 |
| glycerol | | | | | | |
| (22:6)* | | | | | | |
| indoleacetate | SF_Beer_wt | 0.027872 | SF_Tomatoes_wt | −0.02612 | 0.147068 | 0.001308 |
| cis-4-decenoyl- | SF_Watermelon_wt | −0.03164 | Olives Freq | 0.026059 | 0.146705 | 0.001345 |
| carnitine (C10:1) | | | | | | |
| glycerol | Juice Freq | −0.02829 | Olives Freq | 0.027705 | 0.146434 | 0.001373 |
| 2′-deoxyuridine | SF_Mandarin_wt | 0.037435 | SF_Butter_wt | 0.036808 | 0.14643 | 0.001373 |
| laurylcarnitine | 1% Milk Freq | −0.0278 | Beef, Veal, | 0.025018 | 0.146188 | 0.001399 |
| (C12) | | | Lamb, Pork, | | | |
| | | | Steak, Golash | | | |
| | | | Freq | | | |
| X - 12015 | Sweet Dry | 0.066731 | Ordinary | 0.03887 | 0.145373 | 0.001489 |
| | Wine, | | Bread or | | | |
| | Cocktails Freq | | Challah Freq | | | |
| pro-hydroxy-pro | SF_Potatoes_wt | 0.021689 | Artificial | −0.02117 | 0.145074 | 0.001523 |
| | | | Sweeteners | | | |
| | | | Freq | | | |
| adipate | SF_Onion_wt | −0.02415 | Coffee Freq | 0.022951 | 0.144738 | 0.001562 |
| malonate | SF_Carrots_wt | −0.02997 | Nuts, | 0.028427 | 0.144528 | 0.001587 |
| | | | almonds, | | | |
| | | | pistachios | | | |
| | | | Freq | | | |
| cystathionine | SF_Bread_wt | −0.02561 | SF_Wholemeal | 0.021596 | 0.144282 | 0.001617 |
| | | | Bread_wt | | | |
| 4-hydroxy- | SF_Coffee_wt | 0.02973 | White or | −0.02389 | 0.144212 | 0.001626 |
| hippurate | | | Brown Sugar | | | |
| | | | Freq | | | |
| eugenol sulfate | White or | −0.01905 | Fries Freq | −0.01801 | 0.143947 | 0.001659 |
| | Brown Sugar | | | | | |
| | Freq | | | | | |
| X - 24812 | SF_Cooked | −0.02807 | Milk or Dark | −0.02795 | 0.143929 | 0.001661 |
| | Sweet | | Chocolate | | | |
| | potato_wt | | Freq | | | |
| 4-guanidino- | SF_Tomatoes_wt | 0.039862 | Garlic Freq | −0.02602 | 0.14386 | 0.00167 |
| butanoate | | | | | | |
| X - 12718 | SF_Carrots_wt | −0.03717 | Mandarin or | 0.033544 | 0.143481 | 0.001718 |
| | | | Clementine | | | |
| | | | Freq | | | |
| X - 24519 | SF_Apple_wt | −0.03448 | Wholemeal or | −0.03432 | 0.142763 | 0.001813 |
| | | | Rye Bread | | | |
| | | | Freq | | | |
| 3-amino-2- | SF_Tomatoes_wt | −0.01139 | SF_Parsley_wt | −0.00927 | 0.142527 | 0.001846 |
| piperidone | | | | | | |
| N6-carbamoyl- | Salty Snacks | 0.022739 | SF_Lentils_wt | −0.02149 | 0.141974 | 0.001923 |
| threonyladenosine | Freq | | | | | |
| 4-imidazoleacetate | Butter Freq | −0.0354 | Ice Cream or | −0.03056 | 0.141514 | 0.00199 |
| | | | Popsicle which | | | |
| | | | contains | | | |
| | | | Dairy Freq | | | |
| corticosterone | Lemon Freq | −0.0317 | Popsicle | 0.023153 | 0.141213 | 0.002035 |
| | | | Without Dairy | | | |
| | | | Freq | | | |
| DSGEGDFXAE | 3% Milk Freq | 0.009434 | Beef, Veal, | 0.007823 | 0.140729 | 0.00211 |
| GGGVR* | | | Lamb, Pork, | | | |
| | | | Steak, Golash | | | |
| | | | Freq | | | |
| 5alpha-pregnan- | SF_Pancake_wt | 0.014378 | Mandarin or | −0.01387 | 0.140105 | 0.002209 |
| 3beta,20beta-diol | | | Clementine | | | |
| monosulfate (1) | | | Freq | | | |
| N-acetylalliin | Diet Yogurt | −0.04313 | SF_Milk_wt | −0.03666 | 0.139872 | 0.002247 |
| | Freq | | | | | |
| salicylate | SF_Cucumber_wt | 0.011543 | Decaffeinated | 0.008387 | 0.138867 | 0.002419 |
| | | | Coffee Freq | | | |
| X - 16570 | SF_Noodles_wt | −0.02171 | SF_Apple_wt | −0.02108 | 0.137998 | 0.002577 |
| 2-hydroxydecanoate | SF_Wine_wt | 0.021025 | 0.5-3% White | −0.01768 | 0.137633 | 0.002647 |
| | | | Cheese, | | | |
| | | | Cottage Freq | | | |
| isovalerylglycine | SF_White | 0.03198 | Chicken or | 0.029919 | 0.137305 | 0.00271 |
| | Cheese_wt | | Turkey | | | |
| | | | Without Skin | | | |
| | | | Freq | | | |
| sphingomyelin | Onion Freq | 0.022353 | SF_Hummus_wt | −0.01848 | 0.137192 | 0.002733 |
| (d18:0/20:0, | | | | | | |
| d16:0/22:0)* | | | | | | |
| alliin | SF_Lettuce_wt | −0.0251 | Red Pepper | 0.024565 | 0.137142 | 0.002742 |
| | | | Freq | | | |
| docosapentaenoate | Wholemeal or | −0.02936 | SF_Tahini_wt | −0.02817 | 0.136995 | 0.002772 |
| (n6 DPA; 22:5n6) | Rye Bread | | | | | |
| | Freq | | | | | |
| dodecadienoate | Fresh | 0.020045 | 1% Milk Freq | −0.01923 | 0.136 | 0.002978 |
| (12:2)* | Vegetable | | | | | |
| | Salad With | | | | | |
| | Dressing or | | | | | |
| | Oil Freq | | | | | |
| 2-methoxyresorcinol | SF_Granola_wt | 0.025113 | Saltine | 0.02198 | 0.135924 | 0.002994 |
| sulfate | | | Crackers or | | | |
| | | | Matzah Freq | | | |
| biliverdin | SF_Rice_wt | 0.037086 | SF_Coffee_wt | −0.02569 | 0.135857 | 0.003008 |
| oleate/vaccenate | Juice Freq | −0.02202 | SF_Chocolate | −0.01927 | 0.135441 | 0.003099 |
| (18:1) | | | cake_wt | | | |
| 1,2-dipalmitoyl- | SF_Dark | −0.01535 | SF_Cereals_wt | 0.010979 | 0.135357 | 0.003118 |
| GPC | Chocolate_wt | | | | | |
| (16:0/16:0) | | | | | | |
| X - 23787 | SF_Sugar_wt | 0.031617 | SF_Avocado_wt | 0.027549 | 0.135264 | 0.003139 |
| 5alpha-androstan- | Fresh | −0.02279 | Artificial | −0.01932 | 0.133775 | 0.003489 |
| 3beta,17alpha- | Vegetable | | Sweeteners | | | |
| diol disulfate | Salad Without | | Freq | | | |
| | Dressing or | | | | | |
| | Oil Freq | | | | | |
| N-acetylleucine | SF_Cereals_wt | −0.04485 | SF_Ice | 0.032503 | 0.133641 | 0.003522 |
| | | | cream_wt | | | |
| X - 16397 | SF_Egg_wt | 0.025108 | Beef or | 0.022294 | 0.132394 | 0.003845 |
| | | | Chicken Soup | | | |
| | | | Freq | | | |
| hypoxanthine | SF_Eggplant | −0.0122 | SF_Melon_wt | −0.01089 | 0.131461 | 0.004104 |
| | Salad_wt | | | | | |
| guanidinosuccinate | SF_Olive | −0.02439 | Fish Cooked, | 0.023118 | 0.131417 | 0.004117 |
| | oil_wt | | Baked or | | | |
| | | | Grilled Freq | | | |
| oleoylcholine | SF_Chocolate_wt | 0.023667 | SF_Salty | 0.021243 | 0.130381 | 0.004423 |
| | | | Cheese_wt | | | |
| X - 11530 | Garlic Freq | −0.0183 | Lemon Freq | −0.01823 | 0.130312 | 0.004445 |
| sphingomyelin | SF_Potatoes_wt | −0.02832 | Fresh | 0.024218 | 0.130187 | 0.004483 |
| (d18:2/16:0, | | | Vegetable | | | |
| d18:1/16:1)* | | | Salad With | | | |
| | | | Dressing or | | | |
| | | | Oil Freq | | | |
| 1-stearoyl-2- | SF_Schnitzel_wt | 0.013675 | Tomato Freq | −0.0135 | 0.128763 | 0.004944 |
| linoleoyl-GPE | | | | | | |
| (18:0/18:2)* | | | | | | |
| phenyllactate | 5-9% Yellow | 0.010507 | SF_Beer_wt | 0.00983 | 0.128661 | 0.004979 |
| (PLA) | Cheese Freq | | | | | |
| methylsuccinate | SF_Potatoes_wt | −0.02378 | SF_Cake_wt | −0.02196 | 0.128601 | 0.004999 |
| X - 18887 | SF_Onion_wt | 0.022219 | Peanuts Freq | 0.016693 | 0.128585 | 0.005005 |
| X - 21286 | Processed | −0.03438 | SF_Garlic_wt | −0.03224 | 0.128465 | 0.005046 |
| | Meat Free | | | | | |
| | Products Freq | | | | | |
| gamma-glutamyl- | SF_Tahini_wt | 0.03179 | Apple Freq | 0.021253 | 0.128462 | 0.005047 |
| citrulline* | | | | | | |
| glycodeoxy- | Oil as an | −0.03584 | SF_Red | −0.03326 | 0.128237 | 0.005125 |
| cholate sulfate | addition for | | pepper_wt | | | |
| | Salads or | | | | | |
| | Stews Freq | | | | | |
| 3-hydroxylaurate | Butter Freq | 0.016163 | Light Bread | −0.015 | 0.12761 | 0.005348 |
| | | | Freq | | | |
| sulfate of | 3-4.5% | −0.04114 | SF_Soda | 0.040143 | 0.12742 | 0.005418 |
| piperine | Pudding, | | water_wt | | | |
| metabolite | Cheese With | | | | | |
| C16H19NO3 | Additions | | | | | |
| (2)* | Freq | | | | | |
| 1-carboxyethyl- | SF_Hummus_wt | −0.01639 | SF_Alfalfa | −0.01565 | 0.127404 | 0.005424 |
| leucine | | | sprouts_wt | | | |
| sebacate | SF_Beef_wt | 0.019127 | Granola or | 0.017914 | 0.126624 | 0.005717 |
| (decanedioate) | | | Bernflaks | | | |
| | | | Freq | | | |
| N-acetylneuraminate | SF_Halva_wt | 0.003387 | SF_Cottage | −0.0025 | 0.126605 | 0.005725 |
| | | | cheese_wt | | | |
| N-formylanthranilic | SF_Omelette_wt | 0.042702 | SF_Tahini_wt | −0.03925 | 0.126161 | 0.005898 |
| acid | | | | | | |
| picolinate | SF_Tea_wt | −0.04566 | SF_Water_wt | −0.03433 | 0.125907 | 0.006 |
| 4-hydroxybenzoate | SF_Cake_wt | 0.025295 | SF_Bread_wt | 0.02429 | 0.125867 | 0.006016 |
| 2- hydroxybehenate | Ordinary | −0.02983 | Popsicle | −0.02733 | 0.12552 | 0.006157 |
| | Bread or | | Without Dairy | | | |
| | Challah Freq | | Freq | | | |
| 5-dodecenoate | SF_Wholemeal | −0.01578 | 3% Milk Freq | 0.011973 | 0.125442 | 0.00619 |
| (12:1n7) | Bread_wt | | | | | |
| X - 12831 | SF_Dried | −0.0172 | SF_Rice_wt | −0.01653 | 0.124968 | 0.006389 |
| | dates_wt | | | | | |
| glycerol 3-phosphate | SF_Egg_wt | 0.009172 | Corn Freq | −0.0061 | 0.124939 | 0.006401 |
| N-palmitoyltaurine | SF_WhiteWheat_g_wt | 0.023787 | SF_Jam_wt | 0.020896 | 0.124466 | 0.006606 |
| octadecadiene | Cooked | 0.034248 | SF_Cookies_wt | 0.034219 | 0.123141 | 0.007211 |
| dioate (C18:2- | Legumes Freq | | | | | |
| DC)* | | | | | | |
| 1-stearoyl-GPE | SF_Tahini_wt | −0.01952 | SF_Rice | 0.019314 | 0.122848 | 0.007351 |
| (18:0) | | | crackers_wt | | | |
| bilirubin (E, E)* | SF_Coffee_wt | −0.02599 | SF_Tomatoes_wt | −0.02077 | 0.122492 | 0.007525 |
| N-acetylthreonine | SF_Vegetable | 0.024584 | SF_Coffee_wt | −0.02401 | 0.12246 | 0.007541 |
| | Salad_wt | | | | | |
| homoarginine | SF_Chicken | 0.031504 | SF_Beef_wt | 0.03141 | 0.122367 | 0.007587 |
| | breast_wt | | | | | |
| tetradecanedioate | Pear Fresh, | −0.03466 | Butter Freq | 0.033617 | 0.122101 | 0.00772 |
| | Cooked or | | | | | |
| | Canned Freq | | | | | |
| 12-HETE | SF_Butter_wt | 0.016857 | SF_Mandarin_wt | 0.015466 | 0.122054 | 0.007743 |
| X - 11843 | SF_Schnitzel_wt | 0.0157 | SF_Rice_wt | 0.014923 | 0.122053 | 0.007744 |
| X - 22771 | SF_Cucumber_wt | −0.03911 | SF_Beer_wt | 0.017638 | 0.121558 | 0.007998 |
| 2,3-dihydroxy- | SF_Milk_wt | −0.02794 | SF_WhiteWheat_g_wt | 0.027391 | 0.121075 | 0.008253 |
| 5-methylthio- | | | | | | |
| 4-pentenoate | | | | | | |
| (DMTPA)* | | | | | | |
| myristoleoyl- | SF_Cooked | 0.022025 | SF_White | −0.01884 | 0.120905 | 0.008345 |
| carnitine | mushrooms_wt | | Cheese_wt | | | |
| (C14:1)* | | | | | | |
| orotidine | SF_Beer_wt | 0.028445 | 0-1.5% | −0.02404 | 0.120458 | 0.008589 |
| | | | Natural | | | |
| | | | Yogurt Freq | | | |
| X - 18345 | SF_Egg_wt | 0.030911 | SF_Bread_wt | −0.03079 | 0.120368 | 0.008639 |
| N-palmitoyl- | SF_Rice | 0.027654 | SF_Coffee_wt | 0.024178 | 0.119525 | 0.009121 |
| sphingadienine | crackers_wt | | | | | |
| (d18:2/16:0)* | | | | | | |
| glutarate | Coffee Freq | 0.025756 | Artificial | 0.023379 | 0.119351 | 0.009224 |
| (pentanedioate) | | | Sweeteners | | | |
| | | | Freq | | | |
| ornithine | SF_Tahini_wt | 0.022321 | Beer Freq | 0.020484 | 0.118976 | 0.009448 |
| 1-palmitoyl-2- | SF_Onion_wt | −0.02585 | Pita Freq | −0.01849 | 0.118899 | 0.009494 |
| linoleoyl-GPE | | | | | | |
| (16:0/18:2) | | | | | | |
| X - 24512 | SF_Water_wt | −0.02674 | Granola or | 0.026242 | 0.118854 | 0.009522 |
| | | | Bernflaks | | | |
| | | | Freq | | | |
| dopamine 3- | Sugar | −0.01984 | Pastrami or | −0.01335 | 0.118245 | 0.009899 |
| O-sulfate | Sweetened | | Smoked Turkey | | | |
| | Chocolate | | Breast Freq | | | |
| | Milk Freq | | | | | |
| isovalerate | SF_Egg_wt | 0.01826 | Shish Kebab | 0.017714 | 0.117447 | 0.010412 |
| | | | in Pita Bread | | | |
| | | | Freq | | | |
| 1-palmitoyl- | SF_Tahini_wt | −0.01407 | Tahini Salad | −0.01099 | 0.116895 | 0.010782 |
| GPG (16:0)* | | | Freq | | | |
| 14-HDoHE/17- | Banana Freq | −0.00468 | Apple Freq | −0.00412 | 0.116778 | 0.010861 |
| HDoHE | | | | | | |
| 1-palmitoyl- | SF_Vegetable | −0.01871 | Peanuts Freq | −0.0177 | 0.116689 | 0.010922 |
| GPI (16:0) | Salad_wt | | | | | |
| trans- | SF_WholeWheat_g_wt | −0.01835 | Fruit Salad | −0.01808 | 0.115519 | 0.011752 |
| urocanate | | | Freq | | | |
| X - 21842 | SF_Cooked | 0.017668 | SF_Tomatoes_wt | −0.01658 | 0.115074 | 0.012083 |
| | beets_wt | | | | | |
| xanthurenate | SF_Tahini_wt | −0.06647 | Pasta or | −0.05233 | 0.114922 | 0.012198 |
| | | | Flakes Freq | | | |
| N-acetylglutamate | Orange or | −0.01167 | Popsicle | −0.01136 | 0.114108 | 0.012828 |
| | Grapefruit | | Without Dairy | | | |
| | Freq | | Freq | | | |
| phospho- | SF_Vegetable | −0.01884 | Sweet Dry | 0.018577 | 0.113243 | 0.013529 |
| ethanolamine | Salad_wt | | Wine, | | | |
| | | | Cocktails Freq | | | |
| 1-(1-enyl- | Alcoholic | 0.038358 | SF_Chocolate_wt | 0.029272 | 0.113202 | 0.013563 |
| palmitoyl)-2- | Drinks Freq | | | | | |
| palmitoyl-GPC | | | | | | |
| (P-16:0/16:0)* | | | | | | |
| hexadecene- | Regular Tea | −0.03191 | 5-9% White | 0.031756 | 0.112974 | 0.013755 |
| dioate (C16:1- | Freq | | Cheese, | | | |
| DC)* | | | Cottage Freq | | | |
| X - 12822 | Onion Freq | −0.02084 | SF_Vegetable | 0.02052 | 0.112564 | 0.014103 |
| | | | Salad_wt | | | |
| X - 21607 | 1% Milk Freq | −0.03116 | SF_Schnitzel_wt | −0.02665 | 0.112114 | 0.014496 |
| epiandrosterone | SF_Rice | −0.0166 | SF_Coffee_wt | −0.01594 | 0.111052 | 0.015459 |
| sulfate | crackers_wt | | | | | |
| 2-keto-3-deoxy- | Granola or | 0.02985 | SF_Persimmon_wt | 0.026952 | 0.110806 | 0.01569 |
| gluconate | Bernflaks | | | | | |
| | Freq | | | | | |
| hydroxy- | Fried Fish | 0.016469 | Falafel in Pita | 0.014964 | 0.110359 | 0.016118 |
| asparagine** | Freq | | Bread Freq | | | |
| uridine | Wholemeal or | 0.005349 | Simple | −0.00523 | 0.110043 | 0.016426 |
| | Rye Bread | | Cookies or | | | |
| | Freq | | Biscuits Freq | | | |
| 5-(galactosyl- | Parsley, | 0.015888 | Chicken or | 0.015293 | 0.109914 | 0.016554 |
| hydroxy)-L-lysine | Celery, | | Turkey With | | | |
| | Fennel, Dill, | | Skin Freq | | | |
| | Cilantro, | | | | | |
| | Green Onion | | | | | |
| | Freq | | | | | |
| ceramide | SF_Cottage | 0.031752 | SF_WholeWheat_g_wt | 0.03106 | 0.109815 | 0.016652 |
| (d16:1/24:1, | cheese_wt | | | | | |
| d18:1/22:1)* | | | | | | |
| glycosyl | Light Bread | −0.03698 | SF_Dark | 0.036105 | 0.108989 | 0.017493 |
| ceramide | Freq | | Chocolate_wt | | | |
| (d18:1/20:0, | | | | | | |
| d16:1/22:0)* | | | | | | |
| 1-stearoyl-2- | SF_Potatoes_wt | −0.01349 | SF_Tahini_wt | −0.01262 | 0.108819 | 0.01767 |
| oleoyl-GPI | | | | | | |
| (18:0/18:1)* | | | | | | |
| X - 12013 | Parsley, | −0.02619 | >=16% Yellow | 0.017607 | 0.10825 | 0.018276 |
| | Celery, | | Cheese Freq | | | |
| | Fennel, Dill, | | | | | |
| | Cilantro, | | | | | |
| | Green Onion | | | | | |
| | Freq | | | | | |
| 3-hydroxydecanoate | Olives Freq | 0.034271 | Fried Fish | 0.029653 | 0.108189 | 0.018342 |
| | | | Freq | | | |
| anthranilate | Herbal Tea | −0.02746 | SF_White | 0.025736 | 0.106492 | 0.020264 |
| | Freq | | Cheese_wt | | | |
| 5-methyluridine | SF_Tahini_wt | 0.021722 | SF_Vegetable | 0.019528 | 0.106348 | 0.020434 |
| (ribothymidine) | | | Salad_wt | | | |
| 5-bromotryptophan | SF_Chocolate_wt | 0.023321 | 0-1.5% | −0.02139 | 0.106233 | 0.020572 |
| | | | Natural | | | |
| | | | Yogurt Freq | | | |
| 1-(1-enyl- | Orange or | 0.027062 | SF_WhiteWheat_g_wt | −0.02363 | 0.106053 | 0.020788 |
| palmitoyl)-2- | Grapefruit | | | | | |
| linoleoyl-GPC | Freq | | | | | |
| (P-16:0/18:2)* | | | | | | |
| 3-hydroxybutyryl- | Parsley, | 0.031457 | Fried Fish | 0.028827 | 0.105791 | 0.021107 |
| carnitine (2) | Celery, | | Freq | | | |
| | Fennel, Dill, | | | | | |
| | Cilantro, | | | | | |
| | Green Onion | | | | | |
| | Freq | | | | | |
| pregnanolone/ | Chicken or | −0.03249 | SF_Wholemeal | 0.028136 | 0.10566 | 0.021268 |
| allopregnanolone | Turkey With | | Roll_wt | | | |
| sulfate | Skin Freq | | | | | |
| X - 24728 | 3% Milk Freq | −0.04381 | SF_Potatoes_wt | 0.034071 | 0.10566 | 0.021268 |
| 1-oleoyl-GPI | Apricot Fresh | 0.029801 | SF_Yellow | −0.02767 | 0.105514 | 0.021449 |
| (18:1)* | or Dry, or | | Cheese_wt | | | |
| | Loquat Freq | | | | | |
| glycine | SF_Schnitzel_wt | −0.01648 | Canned Tuna | −0.01561 | 0.105187 | 0.021858 |
| | | | or Tuna Salad | | | |
| | | | Freq | | | |
| dihomo- | Nuts, | 0.010126 | Yeast Cakes | −0.00969 | 0.103924 | 0.023504 |
| linoleate | almonds, | | and Cookies | | | |
| (20:2n6) | pistachios | | as Rogallach, | | | |
| | Freq | | Croissant or | | | |
| | | | Donut Freq | | | |
| 2-linoleoyl- | Pita Freq | 0.012823 | SF_Potatoes_wt | 0.012306 | 0.103746 | 0.023745 |
| glycerol (18:2) | | | | | | |
| citrulline | 0-1.5% | 0.021639 | SF_Tomatoes_wt | −0.02144 | 0.103745 | 0.023746 |
| | Natural | | | | | |
| | Yogurt Freq | | | | | |
| lactosyl-N- | SF_Peanuts_wt | 0.034836 | SF_Cooked | −0.03181 | 0.103546 | 0.024017 |
| behenoyl- | | | beets_wt | | | |
| sphingosine | | | | | | |
| (d18:1/22:0)* | | | | | | |
| 1-palmitoleoyl- | Olives Freq | −0.02966 | SF_Jam_wt | 0.024678 | 0.103434 | 0.024171 |
| 2-linolenoyl- | | | | | | |
| GPC | | | | | | |
| (16:1/18:3)* | | | | | | |
| bilirubin (Z, Z) | SF_Beer_wt | 0.013136 | SF_Coffee_wt | −0.01181 | 0.10337 | 0.024259 |
| 4-acetamido- | Coated or | −0.02198 | Yeast Cakes | −0.01416 | 0.10241 | 0.025617 |
| benzoate | Stuffed | | and Cookies | | | |
| | Cookies, | | as Rogallach, | | | |
| | Waffles or | | Croissant or | | | |
| | Biscuits Freq | | Donut Freq | | | |
| docosadienoate | Apricot Fresh | 0.015876 | Yeast Cakes | −0.01526 | 0.102118 | 0.026043 |
| (22:2n6) | or Dry, or | | and Cookies | | | |
| | Loquat Freq | | as Rogallach, | | | |
| | | | Croissant or | | | |
| | | | Donut Freq | | | |
| vanillactate | SF_Wholemeal | 0.036466 | Green Tea | −0.03533 | 0.101992 | 0.026229 |
| | Bread_wt | | Freq | | | |
| taurodeoxy- | SF_WhiteWheat_g_wt | −0.04848 | Peanuts Freq | −0.04794 | 0.101769 | 0.02656 |
| cholic acid 3- | | | | | | |
| sulfate | | | | | | |
| X - 12126 | Ordinary | −0.04479 | Parsley, | −0.03788 | 0.101316 | 0.027245 |
| | Bread or | | Celery, | | | |
| | Challah Freq | | Fennel, Dill, | | | |
| | | | Cilantro, | | | |
| | | | Green Onion | | | |
| | | | Freq | | | |
| stearate (18:0) | SF_Noodles_wt | −0.00834 | SF_Butter_wt | 0.008276 | 0.101288 | 0.027287 |
| indolelactate | SF_WhiteWheat_g_wt | 0.012924 | SF_French | −0.01089 | 0.10121 | 0.027407 |
| | | | fries_wt | | | |
| X - 13684 | SF_Wholemeal | 0.033256 | Fried Fish | 0.01912 | 0.100529 | 0.028469 |
| | Bread_wt | | Freq | | | |
| sulfate of | Red Pepper | 0.033295 | 0.5-3% White | −0.02573 | 0.100095 | 0.029165 |
| piperine | Freq | | Cheese, | | | |
| metabolite | | | Cottage Freq | | | |
| C16H19NO3 | | | | | | |
| (3)* | | | | | | |
| X - 24309 | SF_Almonds_wt | −0.03293 | 5-9% White | 0.027594 | 0.099928 | 0.029436 |
| | | | Cheese, | | | |
| | | | Cottage Freq | | | |
| 1-(1-enyl- | SF_Milk_wt | 0.018166 | Falafel in Pita | −0.01552 | 0.099434 | 0.030253 |
| palmitoyl)-2- | | | Bread Freq | | | |
| palmitoleoyl-GPC | | | | | | |
| (P-16:0/16:1)* | | | | | | |
| N-acetyl-S- | Avocado Freq | −0.01643 | SF_Bamba_wt | 0.016076 | 0.099093 | 0.030825 |
| allyl-L-cysteine | | | | | | |
| 2-oxoarginine* | White or | −0.02507 | SF_Olives_wt | −0.01742 | 0.09899 | 0.031002 |
| | Brown Sugar | | | | | |
| | Freq | | | | | |
| dihomo- | Chicken or | −0.01601 | SF_Wholemeal | 0.010357 | 0.098964 | 0.031045 |
| linolenate | Turkey | | Light | | | |
| (20:3n3 or n6) | Without Skin | | Bread_wt | | | |
| | Freq | | | | | |
| glycochenode | 0.5-3% White | −0.02931 | Simple | 0.022725 | 0.098913 | 0.031134 |
| oxycholate | Cheese, | | Cookies or | | | |
| glucuronide | Cottage Freq | | Biscuits Freq | | | |
| (1) | | | | | | |
| N,N-dimethyl-5- | Wholemeal or | 0.015529 | SF_Milk_wt | −0.01415 | 0.098818 | 0.031297 |
| aminovalerate | Rye Bread | | | | | |
| | Freq | | | | | |
| taurocholate | Sausages Freq | −0.0284 | SF_Cucumber_wt | 0.027816 | 0.09862 | 0.031639 |
| 2-hydroxyadipate | SF_Hamburger_wt | 0.031487 | SF_Cold | 0.031274 | 0.097762 | 0.033158 |
| | | | cut_wt | | | |
| mannose | Cornflakes | −0.01579 | SF_Danish_wt | −0.01488 | 0.097214 | 0.034161 |
| | Freq | | | | | |
| X - 19561 | SF_Tahini_wt | −0.0368 | Salty Cheese, | 0.033886 | 0.097147 | 0.034286 |
| | | | Tzfatit, | | | |
| | | | Bulgarian, | | | |
| | | | Brinza, Thick | | | |
| | | | Slice Freq | | | |
| N-acetylalanine | Apple Freq | 0.01048 | SF_Whipped | 0.009811 | 0.096869 | 0.034806 |
| | | | cream_wt | | | |
| phenylpyruvate | SF_Fried | −0.00521 | Simple | 0.003618 | 0.096291 | 0.035909 |
| | eggplant_wt | | Cookies or | | | |
| | | | Biscuits Freq | | | |
| stearoylcholine* | SF_Hummus | 0.026213 | SF_Chocolate_wt | 0.024884 | 0.096042 | 0.036393 |
| | Salad_wt | | | | | |
| palmitoleoyl- | Lettuce Freq | 0.021993 | SF_Yellow | 0.021945 | 0.095522 | 0.037422 |
| carnitine | | | Cheese_wt | | | |
| (C16:1)* | | | | | | |
| 2-palmitoleoyl- | SF_Onion_wt | −0.02989 | SF_Lettuce_wt | 0.027198 | 0.095476 | 0.037514 |
| GPC (16:1)* | | | | | | |
| phenol sulfate | SF_Potatoes_wt | −0.03138 | SF_Tea_wt | −0.02332 | 0.095336 | 0.037796 |
| X - 23739 | Beer Freq | 0.007641 | SF_Rice | 0.006021 | 0.095281 | 0.037908 |
| | | | crackers_wt | | | |
| 2-stearoyl-GPE | SF_Rice | 0.017055 | Vegetable | 0.016613 | 0.095078 | 0.03832 |
| (18:0)* | crackers_wt | | Soup Freq | | | |
| glycerate | Cooked | 0.018335 | SF_Apple_wt | 0.016299 | 0.094938 | 0.038608 |
| | Vegetable | | | | | |
| | Salads Freq | | | | | |
| X - 12100 | 0-1.5% | 0.007183 | SF_Natural | 0.005165 | 0.094616 | 0.039274 |
| | Natural | | Yogurt_wt | | | |
| | Yogurt Freq | | | | | |
| 5alpha-pregnan- | Brussels | −0.02388 | SF_Vegetable | −0.02263 | 0.094124 | 0.040313 |
| 3beta,20alpha- | Sprouts, | | Salad_wt | | | |
| diol disulfate | Green or Red | | | | | |
| | Cabbage Freq | | | | | |
| phenylalanyl- | SF_Tofu_wt | 0.039139 | Onion Freq | 0.031693 | 0.093617 | 0.041406 |
| glycine | | | | | | |
| heptanoate | SF_WhiteWheat_g_wt | −0.02279 | SF_Sushi_wt | −0.02068 | 0.093589 | 0.041468 |
| (7:0) | | | | | | |
| 4-acetamido- | SF_Chicken | −0.02032 | SF_Apple_wt | 0.019891 | 0.093556 | 0.041539 |
| butanoate | breast_wt | | | | | |
| thyroxine | SF_Wholemeal | 0.027614 | Beef, Veal, | −0.02723 | 0.093455 | 0.041761 |
| | Light | | Lamb, Pork, | | | |
| | Bread_wt | | Steak, Golash | | | |
| | | | Freq | | | |
| 1-oleoyl-GPC | Thousand | −0.01683 | Alcoholic | 0.016764 | 0.093184 | 0.042361 |
| (18:1) | Island | | Drinks Freq | | | |
| | Dressing, | | | | | |
| | Garlic | | | | | |
| | Dressing Freq | | | | | |
| linoleate | Juice Freq | −0.01873 | SF_Cereals_wt | −0.01404 | 0.092187 | 0.044627 |
| (18:2n6) | | | | | | |
| galactonate | SF_Natural | 0.035565 | SF_Cucumber_wt | 0.021854 | 0.091788 | 0.045563 |
| | Yogurt_wt | | | | | |
| octanoyl- | SF_Cooked | 0.019953 | SF_Coffee_wt | −0.01683 | 0.091768 | 0.04561 |
| carnitine (C8) | mushrooms_wt | | | | | |
| piperine | SF_Beer_wt | 0.021738 | Peach, | −0.01748 | 0.091715 | 0.045735 |
| | | | Nectarine, | | | |
| | | | Plum Freq | | | |
| N-acetylproline | Potatoes | −0.0287 | SF_Coffee_wt | 0.023609 | 0.091347 | 0.046616 |
| | Boiled, | | | | | |
| | Baked, | | | | | |
| | Mashed, | | | | | |
| | Potatoes | | | | | |
| | Salad Freq | | | | | |
| X - 12216 | SF_Water_wt | 0.030604 | Roll or | −0.02626 | 0.09095 | 0.047581 |
| | | | Bageles Freq | | | |
| 2-hydroxyglutarate | SF_Rice | 0.018396 | SF_Apple_wt | 0.018065 | 0.090942 | 0.0476 |
| | crackers_wt | | | | | |
| choline | Turkey | −0.00423 | SF_Rice_wt | 0.004025 | 0.090928 | 0.047634 |
| | Meatballs, | | | | | |
| | Beef, Chicken | | | | | |
| | Freq | | | | | |
| 2,2′-Methylene- | SF_Vegetable | 0.057288 | SF_Cake_wt | −0.05264 | 0.090651 | 0.048319 |
| bis(6-tert- | Salad_wt | | | | | |
| butyl-p-cresol) | | | | | | |
| 5,6-dihydrouridine | Turkey | −0.02117 | SF_Diet | −0.01249 | 0.09055 | 0.04857 |
| | Meatballs, | | Coke_wt | | | |
| | Beef, Chicken | | | | | |
| | Freq | | | | | |
| cis-4-decenoate | SF_Low fat | −0.01521 | Salty Cheese, | −0.01403 | 0.090157 | 0.04956 |
| (10:1n6)* | Milk_wt | | Tzfatit, | | | |
| | | | Bulgarian, | | | |
| | | | Brinza, Thick | | | |
| | | | Slice Freq | | | |

Food types that can be used for predicting the corresponding metabolite are also recited in Tables 3 and 4.

The analysis of the frequency of consumption of the food types and/or the daily mean consumption of the food types is optionally and preferably by executing a machine learning procedure. Any of the aforementioned types of machine learning procedures can be used for predicting the quantity of the metabolite based on the food types and/or the daily mean consumption of the food types.

When the metabolite is predicted based on the frequency of consumption and/or the daily mean consumption of the food types, the machine learning procedure used is a trained machine learning procedure. A machine learning procedure can be trained according to some embodiments of the present invention by feeding a machine learning training program with the frequency and/or the daily mean of food types consumed by a cohort of subjects from which the quantities of the metabolite have been determined by blood tests. Once the data are fed, the machine learning training program generates a trained machine learning procedure of a selected type which can then be used without the need to re-train it.

For example, when it is desired to employ decision trees, machine learning training program learns the structure of each tree in a plurality of decision trees (e.g., how many nodes there are in each tree, and how these are connected to one another), and also selects the decision rules for split nodes of each tree. At least a portion of the decision rules relate to one or more food types. A simple decision rule may be a threshold for the frequency of consumption and/or the daily mean consumption of a particular food type, but more complex rules, relating to more than one food type are also contemplated. The machine learning training program also accumulates data at the leaves of the trees.

The structures of the trees, the decision rules for the split nodes, and the data at the leaves are all selected by the machine learning training program, automatically and typically without user intervention, such that the frequency of consumption and/or the daily mean consumption of the food types at the root of the trees provide the quantities of the metabolite as determined by blood tests at the leaves of the trees. The final result of the machine learning training program in this case is a set of trees for each metabolite, where the structures, the decision rules for split nodes, and leaf data for each trees are defined by the machine learning training program.

The Examples section that follows describes machine learning training that was used to generate a set of trees for each of a plurality of metabolite, using training data including metabolite quantities and diet data collected from a cohort of about 500 subjects.

In various exemplary embodiments of the invention a library of machine learning procedures is accessed and searched for a trained machine learning procedure associated with the metabolite. It was found by the inventors that different libraries of machine learning procedures are suitable for microbiome data and for diet data. Thus, when the metabolite is predicted based on the frequency of consumption and/or the daily mean consumption of the food types, the library on medium 110 that is used is preferably not the same as the library used for predicting the metabolite based on the microbiome.

When the metabolite is predicted based on the frequency of consumption and/or the daily mean consumption of the food types, the library can include a machine learning procedure for each of the aforementioned metabolites (in which case N equals the number of the aforementioned metabolites), or a machine learning procedure for each of the metabolites set forth in Table 3 (in which case N equals the number of the metabolites set forth in Table 3), or a machine learning procedure for each of the metabolites set forth in Table 4 (in which case N equals the number of the metabolites set forth in Table 4). Also contemplated are embodiments in which the library includes a machine learning procedure for each of a subset of the aforementioned metabolites or of the metabolites in set forth Table 3, or of the metabolites in set forth Table 4.

FIG. 13 illustrates a machine learning procedure 114 which is the Lth (1≤L≤N) procedure in the library, and which is associated with the metabolite of which the quantity in the blood of the subject is to be predicted. The selected trained procedure 114 is fed with the frequency of consumption and/or the daily mean consumption of the food types, and provides an output indicative of the quantity of the metabolite in the blood.

When machine learning procedure 114 includes a set of decision trees, each of the trees receives food consumption data (typically frequency of consumption and/or the daily mean consumption of the food types), processes the received food consumption data by the split node decision rules that were defined during the training phase, and provides output values in accordance with the data at the leaves that were also defined during the training phase. The output of all trees is optionally and preferably combined (e.g., summed) to provide the quantity of the respective metabolite.

Preferably, the number of trees in the set is at least 1000 or at least 2000 or more. It was found by the inventors that the food types listed in Table 3 dominate the predicting ability of the decision trees. Thus, in some embodiments of the present invention the number of decision rules relating to the food types listed in Table 3 for the respective metabolite is larger than the number of decision rules relating to other food types.

The Inventors found that the machine learning procedures, particularly, but not exclusively the decision trees, can also be used for solving the inverse problem, wherein the machine learning procedure can recommend one or more amounts of microbiomes of an individual, or recommend consumption of one or more food types.

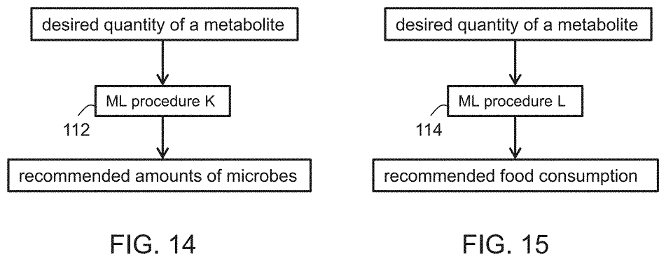

These embodiments are illustrated in FIG. 14 for the case in which the machine learning procedure recommends one or more amounts of microbiomes, and in FIG. 15 for the case in which the machine learning procedure recommends one or more food types.

With reference to FIGS. 11 and 14, the computer readable medium 110 storing a library of machine learning procedures trained using microbiome data is accessed. The library of trained machine learning procedures is searched for a trained machine learning procedure 112 associated with a metabolite of interest. The selected procedure 112 is then fed with a predetermined quantity of the metabolite of interest and provides an output indicative of recommended amounts of a plurality of microbes of a microbiome. The recommended amounts are amounts that would have resulted, within a tolerance of less than 10%, in the predetermined quantity of the metabolite of interest had the amounts been fed to a trained machine learning procedure associated with the metabolite of interest.

With reference to FIGS. 11 and 15, the computer readable medium 110 storing a library of machine learning procedures trained using frequency and/or the daily mean consumption of the food types is accessed. The library of trained machine learning procedures is searched for a trained machine learning procedure 114 associated with a metabolite of interest. The selected procedure 114 is then fed with a predetermined quantity of the metabolite of interest and provides an output indicative of recommended food consumption, typically a recommended set of food types and optionally a recommended consumption frequency and/or daily mean consumption of food types. The recommended food consumption is food consumption that would have resulted, within a tolerance of less than 10%, in the predetermined quantity of the metabolite of interest had the amounts been fed to a trained machine learning procedure associated with the metabolite of interest.

It was surprisingly found by the Inventors that a trained machine learning procedure that solves the forward problem, wherein the procedure provides a metabolite quantity after beaning fed with microbiome data (FIG. 12), or after being fed with consumption frequency and/or daily mean consumption of food types (FIG. 13), can also be used, optionally and preferably without being re-trained, to solve the backward problem, wherein the procedure provides amounts of microbes (FIG. 14) or food consumption (FIG. 15) after being fed with a metabolite quantity.

It will be appreciated that additional features may be used together with the information regarding bacterial abundance and/or food intake to raise the confidence level of the prediction. Such features include for example a macronutrients feature group which can include the daily mean consumption of macronutrients (lipids, proteins, carbohydrates), calories and water, calculated from real-time logging; an anthropometrics feature group which can include weight, BMI, waist and hips circumference, and waist to hips ratio (WHR); a cardiometabolic feature group which can include systolic and diastolic blood pressure, heart rate in beats per minute and a glycemic status; a lifestyle feature group which can include smoking status (current, past) from questionnaires, and the daily mean sleeping time, exercise time and midday sleep time based on the real time logging; a “drugs” feature group which can included binary features representing the reported medication intake of common drugs from questionnaires, and medication groups; a “time of day” feature which is a binary feature indicating whether the sample was taken during the first half of the day; a “seasonal effects” feature which is the month in which the sample was taken, and may also be also grouped months by season (Winter: December-February; Spring: March-May; Summer: June-August; Fall: September-November).

Once the prediction has been made about the metabolite, the present inventors contemplate corroborating the quantity of the metabolite by directly analyzing the amount of that metabolite in the blood of the subject. It is to be understood, however, that while such corroboration is contemplated in some embodiments of the present invention, the corroboration not necessary for the prediction itself. As demonstrated in the Example section that follows, the present inventors were able to train a machine learning procedure such that when fed by the input data (e.g., microbiome data, food consumption data) machine learning procedure, once trained, is capable of predicting the quantity of the metabolite in the blood of the subject even without performing direct analysis of the quantity of the metabolite in the blood of the subject.

Direct analysis of the quantity of the metabolite in the blood of the subject can be performed, for example, during or after the training of the machine learning procedure in order to determine whether the quantity of the metabolite that the machine learning procedure predicts is of clinical relevance, e.g. with a confidence level of at least 90% or at least 95%.

The confidence level of the metabolite quantity can be affirmed by conducting a hypothesis test as known in the art. Typically, the hypothesis test includes selecting the null and alternative hypotheses, and also selecting decision criteria, which are factors upon which a decision to reject or fail to reject the null hypothesis is based. Typical decision criteria include a choice of a test statistic and significance level (denoted algebraically as “alpha”) to be applied to the analysis. Many different test statistics can be used in hypothesis testing, including mean, variance and the like. A p-value can be calculated and be compared to the significance level. The p-value is quantitative assessment of the probability of observing a value of the test statistic that is either as extreme as or more extreme than the calculated value of the test statistic.

Once it is established that a particular trained machine learning procedure is capable of providing clinically relevant predictions for a particular metabolite, the trained machine learning procedure can execute without performing direct analysis of the quantity of the metabolite in the blood of the subject.

Following is a description of techniques suitable for corroborating the quantity of the metabolite in the blood of the subject by direct analysis.

In one embodiment, metabolites are identified using a physical separation method.

The term “physical separation method” as used herein refers to any method known to those with skill in the art sufficient to produce a profile of changes and differences in small molecules produced in hSLCs, contacted with a toxic, teratogenic or test chemical compound according to the methods of this invention. In a preferred embodiment, physical separation methods permit detection of cellular metabolites including but not limited to sugars, organic acids, amino acids, fatty acids, hormones, vitamins, and oligopeptides, as well as ionic fragments thereof and low molecular weight compounds (preferably with a molecular weight less than 3000 Daltons, and more particularly between 50 and 3000 Daltons). For example, mass spectrometry can be used. In particular embodiments, this analysis is performed by liquid chromatography/electrospray ionization time of flight mass spectrometry (LC/ESI-TOF-MS), however it will be understood that metabolites as set forth herein can be detected using alternative spectrometry methods or other methods known in the art for analyzing these types of compounds in this size range.

Certain metabolites can be identified by, for example, gene expression analysis, including real-time PCR, RT-PCR, Northern analysis, and in situ hybridization.

In addition, metabolites can be identified using Mass Spectrometry such as MALDI/TOF (time-of-flight), SELDI/TOF, liquid chromatography-mass spectrometry (LC-MS), gas chromatography-mass spectrometry (GC-MS), high performance liquid chromatography-mass spectrometry (HPLC-MS), capillary electrophoresis-mass spectrometry, nuclear magnetic resonance spectrometry, tandem mass spectrometry (e.g., MS/MS, MS/MS/MS, ESI-MS/MS etc.), secondary ion mass spectrometry (SIMS), or ion mobility spectrometry (e.g. GC-IMS, IMS-MS, LC-IMS, LC-IMS-MS etc.).

Mass spectrometry methods are well known in the art and have been used to quantify and/or identify biomolecules, such as proteins and other cellular metabolites (see, e.g., Li et al., 2000; Rowley et al., 2000; and Kuster and Mann, 1998).

In certain embodiments, a gas phase ion spectrophotometer is used. In other embodiments, laser-desorption/ionization mass spectrometry is used to identify metabolites. Modern laser desorption/ionization mass spectrometry (“LDI-MS”) can be practiced in two main variations: matrix assisted laser desorption/ionization (“MALDI”) mass spectrometry and surface-enhanced laser desorption/ionization (“SELDI”).

In MALDI, the metabolite is mixed with a solution containing a matrix, and a drop of the liquid is placed on the surface of a substrate. The matrix solution then co-crystallizes with the biomarkers. The substrate is inserted into the mass spectrometer. Laser energy is directed to the substrate surface where it desorbs and ionizes the proteins without significantly fragmenting them. However, MALDI has limitations as an analytical tool. It does not provide means for fractionating the biological fluid, and the matrix material can interfere with detection, especially for low molecular weight analytes.

In SELDI, the substrate surface is modified so that it is an active participant in the desorption process. In one variant, the surface is derivatized with adsorbent and/or capture reagents that selectively bind the biomarker of interest. In another variant, the surface is derivatized with energy absorbing molecules that are not desorbed when struck with the laser. In another variant, the surface is derivatized with molecules that bind the biomarker of interest and that contain a photolytic bond that is broken upon application of the laser. In each of these methods, the derivatizing agent generally is localized to a specific location on the substrate surface where the sample is applied. The two methods can be combined by, for example, using a SELDI affinity surface to capture an analyte (e.g. biomarker) and adding matrix-containing liquid to the captured analyte to provide the energy absorbing material.

For additional information regarding mass spectrometers, see, e.g., Principles of Instrumental Analysis, 3rd edition., Skoog, Saunders College Publishing, Philadelphia, 1985; and Kirk-Othmer Encyclopedia of Chemical Technology, 4.sup.th ed. Vol. 15 (John Wiley & Sons, New York 1995), pp. 1071-1094.

In some embodiments, the data from mass spectrometry is represented as a mass chromatogram. A “mass chromatogram” is a representation of mass spectrometry data as a chromatogram, where the x-axis represents time and the y-axis represents signal intensity. In one aspect the mass chromatogram is a total ion current (TIC) chromatogram. In another aspect, the mass chromatogram is a base peak chromatogram. In other embodiments, the mass chromatogram is a selected ion monitoring (SIM) chromatogram. In yet another embodiment, the mass chromatogram is a selected reaction monitoring (SRM) chromatogram. In one embodiment, the mass chromatogram is an extracted ion chromatogram (EIC).

In an EIC, a single feature is monitored throughout the entire run. The total intensity or base peak intensity within a mass tolerance window around a particular analyte's mass-to-charge ratio is plotted at every point in the analysis. The size of the mass tolerance window typically depends on the mass accuracy and mass resolution of the instrument collecting the data. As used herein, the term “feature” refers to a single small metabolite, or a fragment of a metabolite. In some embodiments, the term feature may also include noise upon further investigation.

Detection of the presence of a metabolite will typically involve detection of signal intensity. This, in turn, can reflect the quantity and character of a biomarker bound to the substrate. For example, in certain embodiments, the signal strength of peak values from spectra of a first sample and a second sample can be compared (e.g., visually, by computer analysis etc.) to determine the relative amounts of particular metabolites. Software programs such as the Biomarker Wizard program (Ciphergen Biosystems, Inc., Fremont, Calif.) can be used to aid in analyzing mass spectra. The mass spectrometers and their techniques are well known.

A person skilled in the art understands that any of the components of a mass spectrometer, e.g., desorption source, mass analyzer, detect, etc., and varied sample preparations can be combined with other suitable components or preparations described herein, or to those known in the art. For example, in some embodiments a control sample may contain heavy atoms, e.g. ¹³C, thereby permitting the test sample to be mixed with the known control sample in the same mass spectrometry run. Good stable isotopic labeling is included.

In one embodiment, a laser desorption time-of-flight (TOF) mass spectrometer is used. In laser desorption mass spectrometry, a substrate with a bound marker is introduced into an inlet system. The marker is desorbed and ionized into the gas phase by laser from the ionization source. The ions generated are collected by an ion optic assembly, and then in a time-of-flight mass analyzer, ions are accelerated through a short high voltage field and let drift into a high vacuum chamber. At the far end of the high vacuum chamber, the accelerated ions strike a sensitive detector surface at a different time. Since the time-of-flight is a function of the mass of the ions, the elapsed time between ion formation and ion detector impact can be used to identify the presence or absence of molecules of specific mass to charge ratio.

In one embodiment of the invention, levels of metabolites are detected by MALDI-TOF mass spectrometry.

Methods of detecting metabolites also include the use of surface plasmon resonance (SPR). The SPR biosensing technology has been combined with MALDI-TOF mass spectrometry for the desorption and identification of metabolites.

Data for statistical analysis can be extracted from chromatograms (spectra of mass signals) using softwares for statistical methods known in the art. “Statistics” is the science of making effective use of numerical data relating to groups of individuals or experiments. Methods for statistical analysis are well-known in the art.

In one embodiment a computer is used for statistical analysis.

In one embodiment, the Agilent MassProfiler or MassProfilerProfessional software is used for statistical analysis. In another embodiment, the Agilent MassHunter software Qual software is used for statistical analysis. In other embodiments, alternative statistical analysis methods can be used. Such other statistical methods include the Analysis of Variance (ANOVA) test, Chi-square test, Correlation test, Factor analysis test, Mann-Whitney U test, Mean square weighted derivation (MSWD), Pearson product-moment correlation coefficient, Regression analysis, Spearman's rank correlation coefficient, Student's T test, Welch's T-test, Tukey's test, and Time series analysis.

In different embodiments signals from mass spectrometry can be transformed in different ways to improve the performance of the method. Either individual signals or summaries of the distributions of signals (such as mean, median or variance) can be so transformed. Possible transformations include taking the logarithm, taking some positive or negative power, for example the square root or inverse, or taking the arcsin (Myers, Classical and Modern Regression with Applications, 2nd edition, Duxbury Press, 1990).

The ability to quantitate the amount of a metabolite allows for the diagnosis of diseases which are known to be associated with an up- or down-regulation of that metabolite.

Thus, according to another aspect of the present invention there is provided a method of diagnosing a disease of a subject comprising predicting the quantity of at least one metabolite which is indicative of the disease, wherein the predicting is carried out as described herein, thereby diagnosing the disease.

As used herein the term “diagnosing” refers to determining presence or absence of a pathology (e.g., a disease, disorder, condition or syndrome), classifying a pathology or a symptom, determining a severity of the pathology, monitoring pathology progression, forecasting an outcome of a pathology and/or prospects of recovery and screening of a subject for a specific disease.

Once the level of the metabolite is measured, it is typically compared to a level of that metabolite in a control subject who is known not to be suffering from said disease. If the amount of the metabolite is significantly up- or down-regulated (e.g. by as much as 1.5 fold, 2 fold, 5 fold, 10 fold or more), then it is indicative that the subject has the disease.

Measuring the amount of the metabolite in the control subject may be carried out prior to, at the same time as, or following measuring the amount of the metabolite of the test subject. Preferably, the abundance of said metabolite is measured in a plurality of control subjects. The data from such measurements may be stored in a database, as further described herein below.

Examples of metabolites whose levels are indicative of diseases include cholesterol (for diagnosis of atherosclerosis, cardio vascular disease (CVD)), and glucose (for diagnosis of diabetes). Particular embodiments of the present invention contemplate a metabolite that is not glucose and is also not cholesterol.

Additional examples of metabolites whose levels are indicative of diseases include trimethylamine N-oxide (TMAO) (for diagnosis of CVD); 3-Carboxy-4-methyl-5-propyl-2-furanpropionic acid (CMPF)—(for diagnosis of chronic kidney disease (CKD)); indoxyl sulfate (for diagnosis of CKD, CVD); and phenylacetylglutamine for diagnosis of CKD, CVD, overall mortality. Additional metabolites which are indicative of disease are listed in Man Lam et al., Journal of Genetics and Genomics 44 (2017) 127e138, the contents of which are incorporated herein by reference.

Examples of diseases that may be diagnosed according to this aspect of the present invention include, but are not limited to atherosclerosis, cardio vascular disease (CVD), metabolic diseases such as diabetes, chronic kidney disease and cancer.

According to some embodiments of the invention, screening of the subject for a specific disease is followed by substantiation of the screen results using gold standard methods. Furthermore, once the disease has been diagnosed, the disease may be treated using methods known in the art, particular to each disease.

It will be appreciated that since the methods describe herein pinpoint particular bacterial functions (e.g. species, genus, families etc.) that contribute to the amount of blood metabolites, the present invention can be used for determining which microbes should be altered in order to bring about a particular effect on a particular blood metabolite.

Thus, according to yet another aspect of the present invention there is provided a method of altering the amount of a metabolite. The method optionally and preferably comprises predicting the amount of the metabolite, and administering to the subject one or more agents which specifically increases or decreases the microbe(s), wherein the agent is selected based on the quantity of the metabolite. The prediction of the metabolite can be done using a machine learning procedure, as described above with respect to FIGS. 11 and 12. Thus, computer readable medium 110 storing the library of machine learning procedures is accessed. The library can be searched for a trained machine learning procedure associated with the metabolite. The amounts of the microbes are fed to the selected procedure, which provides an output indicative of the quantity of the metabolite in the blood.

The microbe(s) of the microbiome to be specifically increased or decreased can be selected, according to some embodiments of the present invention, using machine learning. This can be done by operating the trained machine learning procedure to solve the aforementioned inverse problem (FIG. 14), in a manner that will now be explained.

Suppose, for example, that a biological microbiota sample is taken from the body of the subject and is analyzed by biological assays. Suppose that the results of the assays show that the biological microbiota sample contains a set of microbes present at a respective set of amounts in the biological microbiota sample. Suppose further that the amounts of microbes found by the biological assays are fed to a machine learning procedure that has been trained using microbiome data and that is associated with a particular metabolite. Suppose further that the machine learning procedure predicts (FIG. 12) a certain quantity of the particular metabolite, that the predicted quantity is clinically unsatisfactory, and that it is desired to alter the quantity of the particular metabolite to a new, desired, quantity. In this case, the desired, quantity of the particular metabolite can be fed to a machine learning procedure (that has been trained using microbiome data and that is associated with the particular metabolite) in a manner that the machine learning procedure propagates backwards to solve the inverse problem and to provide a set of recommended amounts of microbes (FIG. 14).

The recommended amounts of microbes found by the machine learning procedure can then be compared to the amounts of microbes found by the biological assays, and the agents that are administered are selected based on this comparison. For example, when for a particular microbe, the recommended amount is less that the amount found by the biological assays, the subject is administered with an agent that increases the amount of that particular microbe. Conversely, when for a particular microbe, the recommended amount is more that the amount found by the biological assays, the subject is administered with an agent that decreases the amount of that particular microbe. Also, when for a particular microbe, the recommended amount is the same or approximately the same (with tolerance of up to 10%) as the amount found by the biological assays, no agent is administered for this microbe.

According to one particular embodiment, the altering is carried out by increasing a bacterial population whose level is predicted to being below the level in a healthy subject. Table 1 provides examples of bacterial populations which positively and negatively correlate with a particular metabolite, predictor 1 being of the most significance and predictor 5 being of the least significance.

For example, according to Table 1, a positive number represents a positive correlation of that microbe with the corresponding metabolite and a negative number represents an inverse correlation of that microbe with the corresponding metabolite. Therefore in order to increase the level of X-16124 for example, agents may be provided which increase the level of F: Eggerthellaceae; and decrease the level of S: Gordonibacter pamelaeae.

Altering the amount of particular metabolites may be beneficial to the health of the subject.

According to a particular embodiment, altering the amount of a metabolite is beneficial for the treatment and/or prevention of a disease. Exemplary diseases include, but are not limited to those described herein above.

The term “treating” refers to inhibiting, preventing or arresting the development of a pathology (disease, disorder or condition) and/or causing the reduction, remission, or regression of a pathology. Those of skill in the art will understand that various methodologies and assays can be used to assess the development of a pathology, and similarly, various methodologies and assays may be used to assess the reduction, remission or regression of a pathology.

As used herein, the term “preventing” refers to keeping a disease, disorder or condition from occurring in a subject who may be at risk for the disease, but has not yet been diagnosed as having the disease.

Upregulation:

An agent which increases the amount of a particular bacteria includes that particular bacteria itself (i.e. a probiotic composition).

The term “probiotic” as used herein, refers to one or more microorganisms which, when administered appropriately, can confer a health benefit on the host or subject and/or reduction of risk and/or symptoms of a disease, disorder, condition, or event in a host organism.

The present invention contemplates an agent which up-regulates at least one strain, 10 strains, 20 strains, 30 strains, 40 strains, 50 strains, 60 strains, 70 strains, 80 strains, 90 strains or all of the strains of the above disclosed species.

In one embodiment, the agent specifically upregulates the specified species of bacteria.

Thus, for example, the agent may increase the amount of the specified bacterial species as compared to at least one other bacterial species of the microbiome of the subject, by at least 2 fold. According to a particular embodiment, the agent upregulates the particular bacterial species by at least 5 fold, 10 fold or more as compared to at least one other bacterial species of the microbiome.

In another embodiment, the agent increases the amount of the specified bacterial species as compared to at least 10% of the total bacterial species of the microbiome of the subject, by at least 2 fold. According to a particular embodiment, the agent upregulates the specified bacterial species by at least 5 fold, 10 fold or more as compared to at least 10% of the total bacterial species of the microbiome of the subject.

In another embodiment, the agent increases the amount of the specified bacterial species as compared to at least 20% of the total bacterial species of the microbiome of the subject, by at least 2 fold. According to a particular embodiment, the agent upregulates the specified bacterial species by at least 5 fold, 10 fold or more as compared to at least 20% of the total bacterial species of the microbiome of the subject.

In another embodiment, the agent increases the amount of the specified bacterial species as compared to at least 30% of the total bacterial species of the microbiome of the subject, by at least 2 fold. According to a particular embodiment, the agent upregulates the specified bacterial species by at least 5 fold, 10 fold or more as compared to at least 30% of the total bacterial species of the microbiome of the subject.

In another embodiment, the agent increases the amount of the specified bacterial species as compared to at least 40% of the total bacterial species of the microbiome of the subject, by at least 2 fold. According to a particular embodiment, the agent upregulates the specified bacterial species by at least 5 fold, 10 fold or more as compared to at least 40% of the total bacterial species of the microbiome of the subject.

In another embodiment, the agent increases the amount of the specified bacterial species as compared to at least 50% of the total bacterial species of the microbiome of the subject, by at least 2 fold. According to a particular embodiment, the agent upregulates the specified bacterial species by at least 5 fold, 10 fold or more as compared to at least 50% of the total bacterial species of the microbiome of the subject.

In another embodiment, the agent increases the amount of the specified bacterial species as compared to at least 60% of the total bacterial species of the microbiome of the subject, by at least 2 fold. According to a particular embodiment, the agent upregulates the specified bacterial species by at least 5 fold, 10 fold or more as compared to at least 60% of the total bacterial species of the microbiome of the subject.

In another embodiment, the agent increases the amount of the specified bacterial species as compared to at least 70% of the total bacterial species of the microbiome of the subject, by at least 2 fold. According to a particular embodiment, the agent upregulates the specified bacterial species by at least 5 fold, 10 fold or more as compared to at least 70% of the total bacterial species of the microbiome of the subject.

In another embodiment, the agent increases the amount of the specified bacterial species as compared to at least 80% of the total bacterial species of the microbiome of the subject, by at least 2 fold. According to a particular embodiment, the agent upregulates the specified bacterial species by at least 5 fold, 10 fold or more as compared to at least 80% of the total bacterial species of the microbiome of the subject.

In another embodiment, the agent increases the amount of the specified bacterial species as compared to at least 90% of the total bacterial species of the microbiome of the subject, by at least 2 fold. According to a particular embodiment, the agent upregulates the specified bacterial species by at least 5 fold, 10 fold or more as compared to at least 90% of the total bacterial species of the microbiome of the subject.

According to an embodiment of this aspect of the present invention, the agent increases the species of bacteria by at least 2 fold as compared to at least one other species of bacteria that belongs to a different genus present in the microbiome.

According to a particular embodiment the agent increases the species of bacteria by at least 5 fold, 10 fold or more as compared to at least one other species of bacteria that belongs to a different genus present in the microbiome.

According to one embodiment, the agent increases the species of bacteria by at least 2 fold as compared to at least one other species of bacteria that belongs to the same genus present in the microbiome.

According to a particular embodiment the agent increases the species of bacteria by at least 5 fold, 10 fold or more as compared to at least one other species of bacteria that belongs to the same genus present in the microbiome.

Preferably, the agents of this aspect of the present invention are capable of increases the growth and/or colonization of the bacterial species.

Exemplary agents that are capable of increasing the specified species include microbial compositions. Such microbial compositions typically do not comprise more than 100 bacterial species, more than 90 bacterial species, more than 80 bacterial species, more than 70 bacterial species, more than 60 bacterial species, more than 50 bacterial species, more than 40 bacterial species, more than 30 bacterial species, more than 20 bacterial species, more than 10 bacterial species, or even more than 5 bacterial species.

The microbial compositions of the present invention are not fecal transplants derived from a healthy subject.

The bacterial compositions can comprise more than one strain of a bacterial species, more than 2 strains of a bacterial species, more than 3 strains of a bacterial species, more than 4 strains of a bacterial species, more than 5 strains of a bacterial species, more than 6 strains of a bacterial species, more than 7 strains of a bacterial species, more than 8 strains of a bacterial species, more than 9 strains of a bacterial species, more than 10 strains of a bacterial species, more than 11 strains of a bacterial species, more than 12 strains of a bacterial species, more than 13 strains of a bacterial species, more than 14 strains of a bacterial species, more than 15 strains of a bacterial species, more than 16 strains of a bacterial species, more than 17 strains of a bacterial species, more than 18 strains of a bacterial species, more than 19 strains of a bacterial species, more than 20 strains of a bacterial species or more.

The present inventors contemplate microbial compositions where more than 10%, 20%, 30%, 40%, 50%, 60%, 70%, 80%, 90% or even 100%, of the bacteria of the composition is bacteria of the specified bacterial species.

The present inventors contemplate any formulation for the microbial compositions so long as the bacterial population within is capable of propagating when administered to the subject.

The compositions of the present invention may be formulated as a food supplement, an enema, a tablet, a capsule or a syringe.

The compositions of the invention can be formulated as a slurry, saline or buffered suspensions (e.g., for an enema, suspended in a buffer or a saline), in a drink (e.g., a milk, yoghurt, a shake, a flavoured drink or equivalent) for oral delivery, and the like.

In alternative embodiments, compositions of the invention can be formulated as an enema product, a spray dried product, reconstituted enema, a small capsule product, a small capsule product suitable for administration to children, a bulb syringe, a bulb syringe suitable for a home enema with a saline addition, a powder product, a powder product in oxygen deprived sachets, a powder product in oxygen deprived sachets that can be added to, for example, a bulb syringe or enema, or a spray dried product in a device that can be attached to a container with an appropriate carrier medium such as yoghurt or milk and that can be directly incorporated and given as a dosing for example for children.

In one embodiment, compositions of the invention can be delivered directly in a carrier medium via a screw-top lid wherein the bacterial material is suspended in the lid and released on twisting the lid straight into the carrier medium.

In alternative embodiments methods of delivery of compositions of the invention include use of bacterial slurries into the bowel, via an enema suspended in saline or a buffer, via a small bowel infusion via a nasoduodenal tube, via a gastrostomy, or by using a colonoscope.

According to still another embodiment, the microbial composition of any of the aspects of the present invention is devoid (or comprises only trace quantities) of fecal material (e.g., fiber).

The probiotic bacteria may be in any suitable form, for example in a powdered dry form. In addition, the probiotic microorganism may have undergone processing in order for it to increase its survival. For example, the microorganism may be coated or encapsulated in a polysaccharide, fat, starch, protein or in a sugar matrix. Standard encapsulation techniques known in the art can be used. For example, techniques discussed in U.S. Pat. No. 6,190,591, which is hereby incorporated by reference in its entirety, may be used.

According to a particular embodiment, the probiotic microorganism composition is formulated in a food product, functional food or nutraceutical.

In some embodiments, a food product, functional food or nutraceutical is or comprises a dairy product. In some embodiments, a dairy product is or comprises a yogurt product. In some embodiments, a dairy product is or comprises a milk product. In some embodiments, a dairy product is or comprises a cheese product. In some embodiments, a food product, functional food or nutraceutical is or comprises a juice or other product derived from fruit. In some embodiments, a food product, functional food or nutraceutical is or comprises a product derived from vegetables. In some embodiments, a food product, functional food or nutraceutical is or comprises a grain product, including but not limited to cereal, crackers, bread, and/or oatmeal. In some embodiments, a food product, functional food or nutraceutical is or comprises a rice product. In some embodiments, a food product, functional food or nutraceutical is or comprises a meat product.

Prior to administration, the subject may be pretreated with an agent which reduces the number of naturally occurring microbes in the microbiome (e.g. by antibiotic treatment). According to a particular embodiment, the treatment significantly eliminates the naturally occurring gut microflora by at least 20%, 30% 40%, 50%, 60%, 70%, 80% or even 90%.

Downregulation:

The present invention contemplates an agent which down-regulates at least one strain, 10% of the strains, 20% of the strains, 30% of the strains, 40% of the strains, 50% of the strains, 60% of the strains, 70% of the strains, 80% of the strains, 90% of the strains or all of the strains of any of the uncovered species recited in Table 1.

Thus, for example, the agent may reduce the amount of the specified bacterial species as compared to at least one other bacterial species of the microbiome of the subject, by at least 2 fold. According to a particular embodiment, the agent downregulates the particular bacterial species by at least 5 fold, 10 fold or more as compared to at least one other bacterial species of the microbiome.

In another embodiment, the agent reduces the amount of the specified bacterial species as compared to at least 10% of the total bacterial species of the microbiome of the subject, by at least 2 fold. According to a particular embodiment, the agent downregulates the specified bacterial species by at least 5 fold, 10 fold or more as compared to at least 10% of the total bacterial species of the microbiome of the subject.

In another embodiment, the agent reduces the amount of the specified bacterial species as compared to at least 20% of the total bacterial species of the microbiome of the subject, by at least 2 fold. According to a particular embodiment, the agent downregulates the specified bacterial species by at least 5 fold, 10 fold or more as compared to at least 20% of the total bacterial species of the microbiome of the subject.

In another embodiment, the agent reduces the amount of the specified bacterial species as compared to at least 30% of the total bacterial species of the microbiome of the subject, by at least 2 fold. According to a particular embodiment, the agent downregulates the specified bacterial species by at least 5 fold, 10 fold or more as compared to at least 30% of the total bacterial species of the microbiome of the subject.

In another embodiment, the agent reduces the amount of the specified bacterial species as compared to at least 40% of the total bacterial species of the microbiome of the subject, by at least 2 fold. According to a particular embodiment, the agent downregulates the specified bacterial species by at least 5 fold, 10 fold or more as compared to at least 40% of the total bacterial species of the microbiome of the subject.

In another embodiment, the agent reduces the amount of the specified bacterial species as compared to at least 50% of the total bacterial species of the microbiome of the subject, by at least 2 fold. According to a particular embodiment, the agent downregulates the specified bacterial species by at least 5 fold, 10 fold or more as compared to at least 50% of the total bacterial species of the microbiome of the subject.

In another embodiment, the agent reduces the amount of the specified bacterial species as compared to at least 60% of the total bacterial species of the microbiome of the subject, by at least 2 fold. According to a particular embodiment, the agent downregulates the specified bacterial species by at least 5 fold, 10 fold or more as compared to at least 60% of the total bacterial species of the microbiome of the subject.

In another embodiment, the agent reduces the amount of the specified bacterial species as compared to at least 70% of the total bacterial species of the microbiome of the subject, by at least 2 fold. According to a particular embodiment, the agent downregulates the specified bacterial species by at least 5 fold, 10 fold or more as compared to at least 70% of the total bacterial species of the microbiome of the subject.

In another embodiment, the agent reduces the amount of the specified bacterial species as compared to at least 80% of the total bacterial species of the microbiome of the subject, by at least 2 fold. According to a particular embodiment, the agent downregulates the specified bacterial species by at least 5 fold, 10 fold or more as compared to at least 80% of the total bacterial species of the microbiome of the subject.

In another embodiment, the agent reduces the amount of the specified bacterial species as compared to at least 90% of the total bacterial species of the microbiome of the subject, by at least 2 fold. According to a particular embodiment, the agent downregulates the specified bacterial species by at least 5 fold, 10 fold or more as compared to at least 90% of the total bacterial species of the microbiome of the subject.

According to an embodiment of this aspect of the present invention, the agent reduces the species of bacteria by at least 2 fold as compared to at least one other species of bacteria that belongs to a different genus present in the microbiome.

According to a particular embodiment the agent reduces the species of bacteria by at least 5 fold, 10 fold or more as compared to at least one other species of bacteria that belongs to a different genus present in the microbiome.

According to one embodiment, the agent reduces the species of bacteria by at least 2 fold as compared to at least one other species of bacteria that belongs to the same genus present in the microbiome.

According to a particular embodiment the agent reduces the species of bacteria by at least 5 fold, 10 fold or more as compared to at least one other species of bacteria that belongs to the same genus present in the microbiome.

Preferably, the agents of this aspect of the present invention are capable of decreasing the growth and/or colonization of the bacterial species.

The agent which downregulates the bacteria that is recited in Tables 1 or 2 may be able to reduce the amount (either absolute or relative amount) and/or activity (either absolute or relative activity) of a particular strain of bacteria.

According to a particular embodiment, the agent specifically downregulates the specified strain.

Thus, in one embodiment, the agent reduces the amount of the specified bacterial strain as compared to at least one other bacterial strain of the microbiome of the subject, by at least 2 fold. According to a particular embodiment, the agent downregulates the particular bacterial strain by at least 5 fold, 10 fold or more as compared to at least one other bacterial strain of the microbiome.

In another embodiment, the agent reduces the amount of the specified bacterial strain as compared to at least 10% of the total bacterial strains of the microbiome of the subject, by at least 2 fold. According to a particular embodiment, the agent downregulates the specified bacterial strain by at least 5 fold, 10 fold or more as compared to at least 10% of the total bacterial strains of the microbiome of the subject.

In another embodiment, the agent reduces the amount of the specified bacterial strain as compared to at least 20% of the total bacterial strains of the microbiome of the subject, by at least 2 fold. According to a particular embodiment, the agent downregulates the specified bacterial strain by at least 5 fold, 10 fold or more as compared to at least 20% of the total bacterial strains of the microbiome of the subject.

In another embodiment, the agent reduces the amount of the specified bacterial strain as compared to at least 30% of the total bacterial strains of the microbiome of the subject, by at least 2 fold. According to a particular embodiment, the agent downregulates the specified bacterial strain by at least 5 fold, 10 fold or more as compared to at least 30% of the total bacterial strains of the microbiome of the subject.

In another embodiment, the agent reduces the amount of the specified bacterial strain as compared to at least 40% of the total bacterial strains of the microbiome of the subject, by at least 2 fold. According to a particular embodiment, the agent downregulates the specified bacterial strain by at least 5 fold, 10 fold or more as compared to at least 40% of the total bacterial strains of the microbiome of the subject.

In another embodiment, the agent reduces the amount of the specified bacterial strain as compared to at least 50% of the total bacterial strains of the microbiome of the subject, by at least 2 fold. According to a particular embodiment, the agent downregulates the specified bacterial strain by at least 5 fold, 10 fold or more as compared to at least 50% of the total bacterial strains of the microbiome of the subject.

In another embodiment, the agent reduces the amount of the specified bacterial strain as compared to at least 60% of the total bacterial strains of the microbiome of the subject, by at least 2 fold. According to a particular embodiment, the agent downregulates the specified bacterial strain by at least 5 fold, 10 fold or more as compared to at least 60% of the total bacterial strains of the microbiome of the subject.

In another embodiment, the agent reduces the amount of the specified bacterial strain as compared to at least 70% of the total bacterial strains of the microbiome of the subject, by at least 2 fold. According to a particular embodiment, the agent downregulates the specified bacterial strain by at least 5 fold, 10 fold or more as compared to at least 70% of the total bacterial strains of the microbiome of the subject.

In another embodiment, the agent reduces the amount of the specified bacterial strain as compared to at least 80% of the total bacterial strains of the microbiome of the subject, by at least 2 fold. According to a particular embodiment, the agent downregulates the specified bacterial strain by at least 5 fold, 10 fold or more as compared to at least 80% of the total bacterial strains of the microbiome of the subject.

In another embodiment, the agent reduces the amount of the specified bacterial strain as compared to at least 90% of the total bacterial strains of the microbiome of the subject, by at least 2 fold. According to a particular embodiment, the agent downregulates the specified bacterial strain by at least 5 fold, 10 fold or more as compared to at least 90% of the total bacterial strains of the microbiome of the subject.

According to an embodiment of this aspect of the present invention, the agent reduces the strain of bacteria by at least 2 fold as compared to at least one other strain of bacteria that belongs to a different species present in the microbiome.

According to a particular embodiment the agent reduces the strain of bacteria by at least 5 fold, 10 fold or more as compared to at least one other strain of bacteria that belongs to a different species present in the microbiome.

According to one embodiment, the agent reduces the strain of bacteria by at least 2 fold as compared to at least one other strain of bacteria that belongs to the same species present in the microbiome.

According to a particular embodiment the agent reduces the strain of bacteria by at least 5 fold, 10 fold or more as compared to at least one other strain of bacteria that belongs to the same species present in the microbiome.

Preferably, the agents of this aspect of the present invention are capable of decreasing the growth and/or colonization of the bacterial strain.

An exemplary agent which is capable of reducing a particular bacterial species or strain is an antibiotic.

As used herein, the term “antibiotic agent” refers to a group of chemical substances, isolated from natural sources or derived from antibiotic agents isolated from natural sources, having a capacity to inhibit growth of, or to destroy bacteria, and other microorganisms, used chiefly in treatment of infectious diseases.

Examples of antibiotics contemplated by the present invention include, but are not limited to Daptomycin; Gemifloxacin; Telavancin; Ceftaroline; Fidaxomicin; Amoxicillin; Ampicillin; Bacampicillin; Carbenicillin; Cloxacillin; Dicloxacillin; Flucloxacillin; Mezlocillin; Nafcillin; Oxacillin; Penicillin G; Penicillin V; Piperacillin; Pivampicillin; Pivmecillinam; Ticarcillin; Aztreonam; Imipenem; Doripenem; Meropenem; Ertapenem; Clindamycin; Lincomycin; Pristinamycin; Quinupristin; Cefacetrile (cephacetrile); Cefadroxil (cefadroxyl); Cefalexin (cephalexin); Cefaloglycin (cephaloglycin); Cefalonium (cephalonium); Cefaloridine (cephaloridine); Cefalotin (cephalothin); Cefapirin (cephapirin); Cefatrizine; Cefazaflur; Cefazedone; Cefazolin (cephazolin); Cefradine (cephradine); Cefroxadine; Ceftezole; Cefaclor; Cefamandole; Cefmetazole; Cefonicid; Cefotetan; Cefoxitin; Cefprozil (cefproxil); Cefuroxime; Cefuzonam; Cefcapene; Cefdaloxime; Cefdinir; Cefditoren; Cefetamet; Cefixime; Cefmenoxime; Cefodizime; Cefotaxime; Cefpimizole; Cefpodoxime; Cefteram; Ceftibuten; Ceftiofur; Ceftiolene; Ceftizoxime; Ceftriaxone; Cefoperazone; Ceftazidime; Cefclidine; Cefepime; Cefluprenam; Cefoselis; Cefozopran; Cefpirome; Cefquinome; Fifth Generation; Ceftobiprole; Ceftaroline; Not Classified; Cefaclomezine; Cefaloram; Cefaparole; Cefcanel; Cefedrolor; Cefempidone; Cefetrizole; Cefivitril; Cefmatilen; Cefmepidium; Cefovecin; Cefoxazole; Cefrotil; Cefsumide; Cefuracetime; Ceftioxide; Azithromycin; Erythromycin; Clarithromycin; Dirithromycin; Roxithromycin; Telithromycin; Amikacin; Gentamicin; Kanamycin; Neomycin; Netilmicin; Paromomycin; Streptomycin; Tobramycin; Flumequine; Nalidixic acid; Oxolinic acid; Piromidic acid; Pipemidic acid; Rosoxacin; Ciprofloxacin; Enoxacin; Lomefloxacin; Nadifloxacin; Norfloxacin; Ofloxacin; Pefloxacin; Rufloxacin; Balofloxacin; Gatifloxacin; Grepafloxacin; Levofloxacin; Moxifloxacin; Pazufloxacin; Sparfloxacin; Temafloxacin; Tosufloxacin; Besifloxacin; Clinafloxacin; Gemifloxacin; Sitafloxacin; Troyafloxacin; Prulifloxacin; Sulfamethizole; Sulfamethoxazole; Sulfisoxazole; Trimethoprim-Sulfamethoxazole; Demeclocycline; Doxycycline; Minocycline; Oxytetracycline; Tetracycline; Tigecycline; Chloramphenicol; Metronidazole; Tinidazole; Nitrofurantoin; Vancomycin; Teicoplanin; Telavancin; Linezolid; Cycloserine 2; Rifampin; Rifabutin; Rifapentine; B acitracin; Polymyxin B; Viomycin; Capreomycin.

Antibacterial agents also include antibacterial peptides. Examples include but are not limited to abaecin; andropin; apidaecins; bombinin; brevinins; buforin II; CAP18; cecropins; ceratotoxin; defensins; dermaseptin; dermcidin; drosomycin; esculentins; indolicidin; LL37; magainin; maximum H5; melittin; moricin; prophenin; protegrin; and or tachyplesins.

According to a particular embodiment, the antibiotic is a non-absorbable antibiotic.

Other agents which are not antibiotics are also contemplated by the present inventors.

Thus the present inventors contemplate the use of bacteriophages to downregulate the disclosed bacterial species/strains.

As used herein, the term “bacteriophage” refers to a virus that infects and replicates within bacteria. Bacteriophages are composed of proteins that encapsulate a genome comprising either DNA or RNA. Bacteriophages replicate within bacteria following the injection of their genome into the bacterial cytoplasm.

In one embodiment, the bacteriophage is a lytic bacteriophage. In another embodiment, the bacteriophage is lysogenic.

In some embodiments, the bacteriophages are used in combination with one or more other bacteriophages. The combinations of bacteriophages can target the same detrimental microorganism or different detrimental microorganisms. Preferably, the combination of bacteriophages targets the same detrimental microorganism.

In some embodiments, the bacteriophage or combination of bacteriophages are used in combination with one or more probiotic microorganisms—such as those described herein below.

In other embodiments, the bacteriophages or combination of bacteriophages are used in combination with one or more antibiotic, as disclosed herein.

In some embodiments, the bacteriophage is administered orally at a dose ranging from 10⁵ to 10¹⁰ plaque-forming units (PFU)/g, preferably 10⁷ to 10⁸ PFU/g. In some embodiments, the bacteriophages are administered at a dose of 10⁵ to 10¹⁰ PFU/day, preferably 10⁷ to 10⁸ PFU/day.

According to another embodiment, the agent is a bacteriophage protein such as an isolated phage protein, e.g., a lysin protein, tail protein, or active fragment.

In one embodiment, the agent which is capable of down-regulating a particular bacterial species/strain is a bacterial population that competes with the bacterial species/strain for essential resources. Bacterial compositions are further described herein below.

In still another embodiment, the agent which is capable of down-regulating a particular bacterial species/strain is a metabolite of a competing bacterial population (or even from the same species/strain) that serves to decrease the relative amount of the bacterial species/strain.

Additional agents that can specifically reduce a particular bacterial species or strain are known in the art and include polynucleotide silencing agents.

Preferably, the polynucleotide silencing agent of this aspect of the present invention targets a sequence that encodes at least one essential gene (i.e., compatible with life) in the bacteria. The sequence which is targeted should be specific to the particular bacteria species that it is desired to down-regulate. Such genes include ribosomal RNA genes (16S and 23S), ribosomal protein genes, tRNA-synthetases, as well as additional genes shown to be essential such as dnaB, fabI, folA, gyrB, murA, pytH, metG, and tufA(B).

According to an embodiment of the invention, the polynucleotide silencing agent is specific to the target RNA and does not cross inhibit or silence other targets or a splice variant which exhibits 99% or less global homology to the target gene, e.g., less than 98%, 97%, 96%, 95%, 94%, 93%, 92%, 91%, 90%, 89%, 88%, 87%, 86%, 85%, 84%, 83%, 82%, 81% global homology to the target gene; as determined by PCR, Western blot, Immunohistochemistry and/or flow cytometry.

One agent capable of downregulating an essential bacterial gene is a RNA-guided endonuclease technology e.g. CRISPR system. In one embodiment, the CRISPR system is expressed in a bacteriophage.

As used herein, the term “CRISPR system” also known as Clustered Regularly Interspaced Short Palindromic Repeats refers collectively to transcripts and other elements involved in the expression of or directing the activity of CRISPR-associated genes, including sequences encoding a Cas gene (e.g. CRISPR-associated endonuclease 9), a tracr (trans-activating CRISPR) sequence (e.g. tracrRNA or an active partial tracrRNA), a tracr-mate sequence (encompassing a “direct repeat” and a tracrRNA-processed partial direct repeat) or a guide sequence (also referred to as a “spacer”) including but not limited to a crRNA sequence (i.e. an endogenous bacterial RNA that confers target specificity yet requires tracrRNA to bind to Cas) or a sgRNA sequence (i.e. single guide RNA).

In some embodiments, one or more elements of a CRISPR system is derived from a type I, type II, or type III CRISPR system. In some embodiments, one or more elements of a CRISPR system (e.g. Cas) is derived from a particular organism comprising an endogenous CRISPR system, such as Streptococcus pyogenes, Neisseria meningitides, Streptococcus thermophilus or Treponema denticola.

In general, a CRISPR system is characterized by elements that promote the formation of a CRISPR complex at the site of a target sequence (also referred to as a protospacer in the context of an endogenous CRISPR system).

In the context of formation of a CRISPR complex, “target sequence” refers to a sequence to which a guide sequence (i.e. guide RNA e.g. sgRNA or crRNA) is designed to have complementarity, where hybridization between a target sequence and a guide sequence promotes the formation of a CRISPR complex. Full complementarity is not necessarily required, provided there is sufficient complementarity to cause hybridization and promote formation of a CRISPR complex. Thus, according to some embodiments, global homology to the target sequence may be of 50%, 60%, 70%, 75%, 80%, 85%, 90%, 95% or 99%. A target sequence may comprise any polynucleotide, such as DNA or RNA polynucleotides. In some embodiments, a target sequence is located in the nucleus or cytoplasm of a cell.

Thus, the CRISPR system comprises two distinct components, a guide RNA (gRNA) that hybridizes with the target sequence, and a nuclease (e.g. Type-II Cas9 protein), wherein the gRNA targets the target sequence and the nuclease (e.g. Cas9 protein) cleaves the target sequence. The guide RNA may comprise a combination of an endogenous bacterial crRNA and tracrRNA, i.e. the gRNA combines the targeting specificity of the crRNA with the scaffolding properties of the tracrRNA (required for Cas9 binding). Alternatively, the guide RNA may be a single guide RNA capable of directly binding Cas.

Typically, in the context of an endogenous CRISPR system, formation of a CRISPR complex (comprising a guide sequence hybridized to a target sequence and complexed with one or more Cas proteins) results in cleavage of one or both strands in or near (e.g. within 1, 2, 3, 4, 5, 6, 7, 8, 9, 10, 20, 50, or more base pairs from) the target sequence. Without wishing to be bound by theory, the tracr sequence, which may comprise or consist of all or a portion of a wild-type tracr sequence (e.g. about or more than about 20, 26, 32, 45, 48, 54, 63, 67, 85, or more nucleotides of a wild-type tracr sequence), may also form part of a CRISPR complex, such as by hybridization along at least a portion of the tracr sequence to all or a portion of a tracr mate sequence that is operably linked to the guide sequence.

In some embodiments, the tracr sequence has sufficient complementarity to a tracr mate sequence to hybridize and participate in formation of a CRISPR complex. As with the target sequence, a complete complementarity is not needed, provided there is sufficient to be functional. In some embodiments, the tracr sequence has at least 50%, 60%, 70%, 80%, 90%, 95% or 99% of sequence complementarity along the length of the tracr mate sequence when optimally aligned.

Introducing CRISPR/Cas into a cell may be effected using one or more vectors driving expression of one or more elements of a CRISPR system such that expression of the elements of the CRISPR system direct formation of a CRISPR complex at one or more target sites. For example, a Cas enzyme, a guide sequence linked to a tracr-mate sequence, and a tracr sequence could each be operably linked to separate regulatory elements on separate vectors. Alternatively, two or more of the elements expressed from the same or different regulatory elements, may be combined in a single vector, with one or more additional vectors providing any components of the CRISPR system not included in the first vector. CRISPR system elements that are combined in a single vector may be arranged in any suitable orientation, such as one element located 5′ with respect to (“upstream” of) or 3′ with respect to (“downstream” of) a second element. The coding sequence of one element may be located on the same or opposite strand of the coding sequence of a second element, and oriented in the same or opposite direction. A single promoter may drive expression of a transcript encoding a CRISPR enzyme and one or more of the guide sequence, tracr mate sequence (optionally operably linked to the guide sequence), and a tracr sequence embedded within one or more intron sequences (e.g. each in a different intron, two or more in at least one intron, or all in a single intron).

As well as altering the bacterial composition of the microbiome of the subject, the present inventors also contemplate altering food intake to control the level of a metabolite.

Thus, according to a particular aspect of the present invention there is provided a method of providing dietary advice to a subject, the method comprising predicting the level of a metabolite in the blood by carrying out the methods described herein, wherein when said metabolite is above or below the recommended level of said metabolite, recommending consumption of at least one food type that alters the level of said metabolite.

The dietary advice can be provided, according to some embodiments of the present invention, using machine learning. This can be done by operating the trained machine learning procedure to solve the aforementioned inverse problem (FIG. 15), in a manner that will now be explained.

Suppose, for example, that for a particular subject it was found that a certain quantity Q1 of a particular metabolite is clinically unsatisfactory, and that it is desired to alter the quantity of the particular metabolite to a new, desired, quantity Q2. The quantity Q1 can be found by performing a blood test or, more preferably, by feeding a machine learning procedure that has been trained using food consumption data and that is associated with a particular metabolite, with the frequency and/or the daily mean consumption of several food types (FIG. 13).

The desired quantity Q2 of the particular metabolite can fed to a machine learning procedure (that has been trained using food consumption data and that is associated with the particular metabolite) in a manner that the machine learning procedure propagates backwards to solve the inverse problem and to provide a recommended food consumption (FIG. 15), typically a recommended set of food types and optionally a recommended consumption frequency and/or daily mean consumption of food types. The recommended food consumption can be used as the dietary advice.

In one embodiment, the metabolite is set forth in Table 3 and more preferably in Table 4.

The dietary advise provided to the subject could include a list of foods that may help in increasing or decreasing that metabolite.

According to one particular embodiment, the altering is carried out by increasing intake of a food whose level is predicted to being below the level in a healthy subject. Table 3 provides examples food types which positively correlate with a particular metabolite.

For example, according to Table 3, in order to increase the level of 1-methylxanthine for example, the amount of coffee intake should be increased.

Tables 3 and 4 list the most preferred foods that can be altered in order to alter the level of the corresponding metabolite, predictor 1 being of the most significance and predictor 5 being of the least significance. Of note, the abbreviation “wt” which appears in the Tables refers to the daily mean consumption of specific food types in grams.

As used herein the term “about” refers to ±10%.

The terms “comprises”, “comprising”, “includes”, “including”, “having” and their conjugates mean “including but not limited to”.

The term “consisting of” means “including and limited to”.

The term “consisting essentially of” means that the composition, method or structure may include additional ingredients, steps and/or parts, but only if the additional ingredients, steps and/or parts do not materially alter the basic and novel characteristics of the claimed composition, method or structure.

As used herein, the singular form “a”, “an” and “the” include plural references unless the context clearly dictates otherwise. For example, the term “a compound” or “at least one compound” may include a plurality of compounds, including mixtures thereof.

Throughout this application, various embodiments of this invention may be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range.

Whenever a numerical range is indicated herein, it is meant to include any cited numeral (fractional or integral) within the indicated range. The phrases “ranging/ranges between” a first indicate number and a second indicate number and “ranging/ranges from” a first indicate number “to” a second indicate number are used herein interchangeably and are meant to include the first and second indicated numbers and all the fractional and integral numerals therebetween.

As used herein the term “method” refers to manners, means, techniques and procedures for accomplishing a given task including, but not limited to, those manners, means, techniques and procedures either known to, or readily developed from known manners, means, techniques and procedures by practitioners of the chemical, pharmacological, biological, biochemical and medical arts.

As used herein, the term “treating” includes abrogating, substantially inhibiting, slowing or reversing the progression of a condition, substantially ameliorating clinical or aesthetical symptoms of a condition or substantially preventing the appearance of clinical or aesthetical symptoms of a condition.

When reference is made to particular sequence listings, such reference is to be understood to also encompass sequences that substantially correspond to its complementary sequence as including minor sequence variations, resulting from, e.g., sequencing errors, cloning errors, or other alterations resulting in base substitution, base deletion or base addition, provided that the frequency of such variations is less than 1 in 50 nucleotides, alternatively, less than 1 in 100 nucleotides, alternatively, less than 1 in 200 nucleotides, alternatively, less than 1 in 500 nucleotides, alternatively, less than 1 in 1000 nucleotides, alternatively, less than 1 in 5,000 nucleotides, alternatively, less than 1 in 10,000 nucleotides.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable subcombination or as suitable in any other described embodiment of the invention. Certain features described in the context of various embodiments are not to be considered essential features of those embodiments, unless the embodiment is inoperative without those elements.

Various embodiments and aspects of the present invention as delineated hereinabove and as claimed in the claims section below find experimental support in the following examples.

### EXAMPLES

Reference is now made to the following examples, which together with the above descriptions illustrate some embodiments of the invention in a non limiting fashion.

Generally, the nomenclature used herein and the laboratory procedures utilized in the present invention include molecular, biochemical, microbiological and recombinant DNA techniques. Such techniques are thoroughly explained in the literature. See, for example, “Molecular Cloning: A laboratory Manual” Sambrook et al., (1989); “Current Protocols in Molecular Biology” Volumes I-III Ausubel, R. M., ed. (1994); Ausubel et al., “Current Protocols in Molecular Biology”, John Wiley and Sons, Baltimore, Md. (1989); Perbal, “A Practical Guide to Molecular Cloning”, John Wiley & Sons, New York (1988); Watson et al., “Recombinant DNA”, Scientific American Books, New York; Birren et al. (eds) “Genome Analysis: A Laboratory Manual Series”, Vols. 1-4, Cold Spring Harbor Laboratory Press, New York (1998); methodologies as set forth in U.S. Pat. Nos. 4,666,828; 4,683,202; 4,801,531; 5,192,659 and 5,272,057; “Cell Biology: A Laboratory Handbook”, Volumes I-III Cellis, J. E., ed. (1994); “Culture of Animal Cells—A Manual of Basic Technique” by Freshney, Wiley-Liss, N. Y. (1994), Third Edition; “Current Protocols in Immunology” Volumes I-III Coligan J. E., ed. (1994); Stites et al. (eds), “Basic and Clinical Immunology” (8th Edition), Appleton & Lange, Norwalk, Conn. (1994); Mishell and Shiigi (eds), “Selected Methods in Cellular Immunology”, W. H. Freeman and Co., New York (1980); available immunoassays are extensively described in the patent and scientific literature, see, for example, U.S. Pat. Nos. 3,791,932; 3,839,153; 3,850,752; 3,850,578; 3,853,987; 3,867,517; 3,879,262; 3,901,654; 3,935,074; 3,984,533; 3,996,345; 4,034,074; 4,098,876; 4,879,219; 5,011,771 and 5,281,521; “Oligonucleotide Synthesis” Gait, M. J., ed. (1984); “Nucleic Acid Hybridization” Hames, B. D., and Higgins S. J., eds. (1985); “Transcription and Translation” Hames, B. D., and Higgins S. J., eds. (1984); “Animal Cell Culture” Freshney, R. I., ed. (1986); “Immobilized Cells and Enzymes” IRL Press, (1986); “A Practical Guide to Molecular Cloning” Perbal, B., (1984) and “Methods in Enzymology” Vol. 1-317, Academic Press; “PCR Protocols: A Guide To Methods And Applications”, Academic Press, San Diego, Calif. (1990); Marshak et al., “Strategies for Protein Purification and Characterization—A Laboratory Course Manual” CSHL Press (1996); all of which are incorporated by reference as if fully set forth herein. Other general references are provided throughout this document. The procedures therein are believed to be well known in the art and are provided for the convenience of the reader. All the information contained therein is incorporated herein by reference.

This Example examines the relationship between levels of serum metabolites and a rich resource of clinical parameters, dietary intake patterns, lifestyle measurements, human genetics and gut microbiota composition across a large healthy cohort. This Example demonstrates that using these features highly accurate out-of-sample predictions for over 1000 circulating serum metabolites can be obtained, with diet and gut microbiome having the highest predictive power, and being particularly predictive for unknown compounds. The inventors uncovered a list of associations between genetic loci and circulating blood metabolites and showed that we replicate several known links between specific SNPs and metabolites. By applying the prediction models of the present embodiments to an independent cohort of 31 participants, the inventors validated many of the associations. Using feature attribution analysis on the resulting predictive models, the inventors uncovered both known and novel associations between diet, gut microbiome and the levels of blood metabolites.

This Example demonstrates that many metabolites are exclusively explained by gut microbiome composition, highlighting its potential as their key determinant, and revealed the identities and predicted candidate structure of many unknown compounds which are highly predictable by the microbiome.

This Example also demonstrates that the uncovered associations are causal, as levels of metabolites were predicted to be positively associated with bread increased following a randomized clinical trial of bread intervention.

This Example concentrates on estimates computed via out-of-sample predictions, since such evaluation of performance is based only on unseen samples as the most strict and conservative estimate of performance. As such, the results presented herein constitute a lower bound for the amount of variance in metabolite levels that may be explained by the various features we examined.

The heterogeneity of the data is advantageous since its estimates do not depend on modeling assumptions.

### Materials and Methods

All statistical and machine learning analyses were performed using Python (version 2.7.8).

Description of Cohorts

We analyzed banked samples from two previously collected cohorts^{25,48}, for a total of 522 Israeli individuals. Studies were approved by Tel Aviv Sourasky Medical Center Institutional Review Board (IRB), approval numbers TLV-0658-12, TLV-0050-13 and TLV-0522-10; Kfar Shaul Hospital IRB, approval number 0-73. All participants signed written informed consent forms. Full study designs, including inclusion and exclusion criteria were described elsewhere^{25,48}. In brief, participants in both studies were healthy individuals aged between 18 and 70. All participants answered detailed medical, lifestyle and nutritional questionnaires, provided stool and serum samples for metagenomic sequencing and metabolomics, were genotyped, underwent a comprehensive blood test, and for a period of at least one week, recorded all of their daily activities and nutritional intake in real-time using their smartphones with a specialized app provided to them⁴⁸.

Feature Groups

The “diet” feature group includes answers for a detailed food frequency questionnaire (FFQ) aimed at capturing long term dietary habits, and the daily mean consumption of different food types, computed over a week based on real-time logging. In both cases we kept only items which were reported to be consumed at least once by at least 1% of our participants, resulting in 670 different food types from logging, and 141 different items from the FFQ.

The “macronutrients” feature group includes the daily mean consumption of macronutrients (lipids, proteins, carbohydrates), calories and water, calculated from real-time logging.

The “anthropometrics” feature group includes weight, BMI, waist and hips circumference, and waist to hips ratio (WHR).

The “cardiometabolic” feature group includes systolic and diastolic blood pressure, heart rate in beats per minute and a glycemic status as previously described³⁰.

The “drugs” feature group includes 30 binary features representing the intake of 20 common medications as reported in questionnaires, in addition to 10 medication groups as previously described³⁰. We included only drugs reported to be used by at least 1% of our participants.

The “clinical data” feature group includes the age and sex of the participants, and the following feature groups described above: anthropometrics, cardiometabolic, and drugs.

The “lifestyle” feature group includes smoking status (current, past), stress levels obtained from questionnaires, and the daily mean sleeping time, exercise time and midday sleep time based on real time logging.

The “time of day” feature is a binary feature indicating whether the sample was taken during the first half of the day.

The “seasonal effects” feature is the month in which the sample was taken. In some analyses we also grouped months by season (Winter: December-February; Spring: March-May; Summer: June-August; Fall: September-November).

The “microbiome” feature group includes bacterial relative abundance calculated both by considering coverage (see below), and by MetaPhlAn2⁵⁵, as well as the first 10 principal components computed over the log transformed relative abundance of a bacterial gene catalog⁵⁶ as previously described³⁰′⁵⁷. Preprocessing steps are described below.

We further defined a full model that included all of the above.

Metabolomics Profiling and Preprocessing Metabolite concentrations were measured in serum samples by Metabolon, Inc., Durham, N.C., USA, by using an untargeted LC/MS platform as previously described⁶′⁵⁸′⁵⁹. A total of 540 serum samples were profiled, 19 of which were control samples (technical replicate) pooled from several individuals. The other 521 serum samples belonged to 491 participants.

We removed from further analysis 27 metabolites with less than 10 measurements across our cohort, and 54 metabolites that we found to have significantly different distributions in samples collected in two different recruitment centers (Mann-Whitney U p<0.05/1251; Bonferroni corrected). For the remaining 1170 metabolites, we performed robust standardization (subtracting the median and dividing by the standard deviation) over the log (base 10) transformed levels, followed by clipping outlier samples which were farther than 5 standard deviations. We next used two separate normalization schemes, one for single metabolites, which we subsequently used in the feature attribution analysis, and the second for metabolite groups, which we used for global and enrichment analyses.

For single metabolites, we regressed metabolite levels against storage times (only for metabolites present in at least 50 samples), and finally, imputed missing values as the minimum value per metabolite. For the second scheme, metabolites were grouped by correlation with a Spearman rho threshold of 0.85. This is done in order to handle possible bias resulting from uncertainty of metabolite assignments and a high rate of highly correlated mass spectrometry peaks, and resulted in 1067 metabolite groups, 982 of which are singletons. The value of the metabolite group was set to the mean. The category of each metabolite group was assigned based on majority vote, where unknown compounds were excluded from the vote unless all metabolites in the group were unknown.

Microbiome Preprocessing

Sample collection, DNA extraction, and sequencing of the samples in this study was described previously^{25,30,48} Briefly, we used only samples which were collected using swabs, filtered metagenomic reads containing Illumina adapters, filtered low-quality reads and trimmed low-quality read edges. We detected host DNA by mapping with GEM⁶⁰ to the human genome (hg19) with inclusive parameters, and removed human reads. We subsampled all samples to have 10 million reads.

Bacterial relative abundance estimation was performed by mapping bacterial reads to species-level genome bins (SGB) representative genomes³³. We selected all SGB representatives with at least 5 genomes in group, and for these representative genomes kept only unique regions as a reference data set. Mapping was performed using bowtie2⁶¹ and abundance was estimated by calculating the mean coverage of unique genomic regions across the 50 percent most densely covered areas as previously described⁵⁷′⁶². Feature names include the lowest taxonomy level identified.

Comparing Metabolomics to Lab Tests

We compared the levels of both creatinine and cholesterol which we previously obtained via standard lab tests²⁵ with their metabolomic levels. Since the lab tests were performed by two different labs, we centered the tests by reducing from the value of each sample the mean of all tests taken in the lab in which it was performed. We then performed a standardization of the resulting measurements. The metabolomic profiling and the lab tests were performed on two samples taken at the same blood draw.

Correlation of Metabolic Profiles within and Between Individuals

We compared the levels of both creatinine and cholesterol which we previously obtained via standard lab tests²⁵ with their metabolomic levels. Since the lab tests were performed by two different labs, we centered the tests by reducing from the value of each sample the mean of all tests taken in the lab in which it was performed. We then performed a standardization of the resulting measurements. The metabolomic profiling and the lab tests were performed on two samples taken at the same blood draw.

Predictive Models of Metabolite Groups

We used gradient boosting decision trees from the LightGBM (version 2.1.2) package²⁷, in order to predict the levels of 1067 metabolite groups based on 7 feature groups in held-out subjects. In order to estimate the EV of each metabolite group we ran a 5-fold cross validation (CV) model using each feature group as input, and evaluated the results using Pearson correlation. For all prediction results we computed 95% confidence intervals and p-values via 1000 iterations of bootstrapping⁶³. In each bootstrap iteration, we performed a random 5-fold cross validation, were in each fold we randomly sampled (with replacement) a group of subjects from the training set to have the same size as the current training set. We next used this set in order to train our model and evaluated the model's performance on the set of subjects in the remaining fold. Finally we computed the Pearson correlation between the measured values of the metabolite and the concatenation of the CV's predicted values as obtained from the bootstrapping iteration. We applied the Fisher transformation to the Pearson correlations we got from bootstrapping in order to induce normality⁶⁴, and then computed a standard error, and estimated the p-values via the normal CDF using the Wald test⁶⁵, such that our null hypothesis is that the correlations should distribute normally with zero mean. Confidence intervals were computed empirically from the bootstrapping correlations. We corrected p-values of predictions for multiple hypotheses using the Bonferroni procedure within each feature group (p<0.05/1067). In all CV and bootstrapping runs we used a fixed and predetermined set of hyperparameters (Table 5).

TABLE 5

| | Microbiome and | Other feature |
|---|---|---|
| | Diet | groups |
| LightGBM HyperParameter | | |
| learning_rate | 0.005 | 0.01 |
| max_depth | default | 5 |
| feature_fraction | 0.2 | 0.8 |
| num_leaves | default | 25 |
| min_data_in_leaf | 15 | 15 |
| metric | 12 | 12 |
| early_stopping_rounds | None | None |
| n_estimators | 2000 | 200 |
| bagging_fraction | 0.8 | 0.9 |
| bagging_freq | 1 | 5 |
| num_threads | 1 | 1 |
| verbose | −1 | −1 |
| silent | TRUE | TRUE |

Testing for SNP Associations with Metabolites

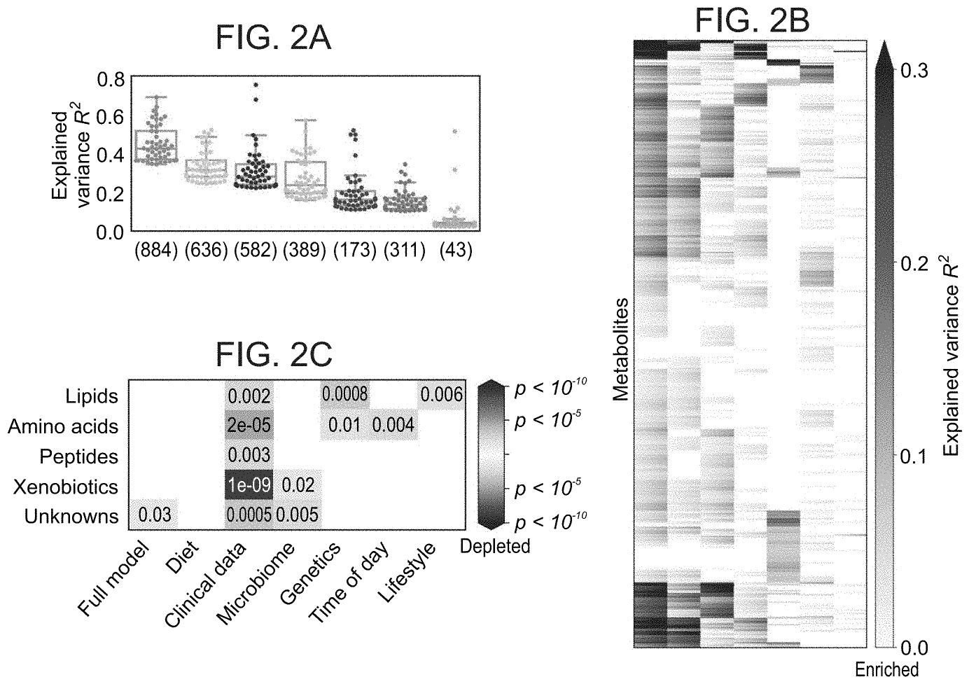

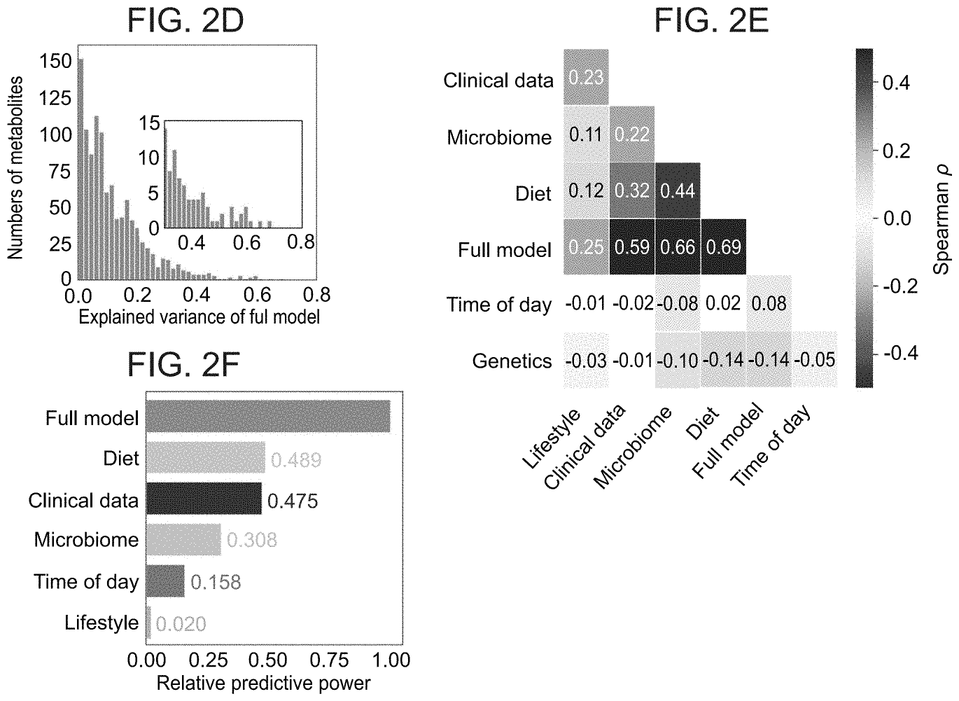

Genotype processing and imputation of 413 individuals were described previously³⁰. We performed genome wide associations for single metabolites (n=1170) and calculated the p-value and the estimated effect sizes using plink (v1.07). When declaring a genome-wide significance for the SNP-metabolite associations we used a conservative Bonferroni adjustment procedure to control for the false discovery rate due to the large number of SNPs tested (p<(5×10⁻⁸)/1170). We performed all genome wide associations using imputed genotypes. Results presented in FIGS. 2A-F are based on a similar analysis performed over the metabolite groups (n=1067).

For the replication of SNP-metabolite associations from a previous study⁶ we correlated the EV of each metabolite from a model based on top significantly associated SNPs in the TwinsUK, and the effect size of the single top significantly associated SNP in this study. Only 301 metabolites which were measured in both studies were considered for analysis.

Pathway Category Enrichment Analysis

For each pathway category we used a Mann-Whitney U test comparing the prediction accuracy of metabolites from that category compared to prediction accuracy of metabolites from other categories. Direction of enrichment was determined by the sign of the Mann-Whitney U test statistic. We considered only metabolite groups for which at least one feature group had a significant prediction (after correcting for multiple hypothesis), resulting with 982 metabolite groups.

Validation of Metabolite Predictions

For every feature group, we trained a prediction model based solely on the samples from the main cohort, and evaluated its performance on the independent validation cohort. In all validation analyses we only considered 877 metabolite groups which were present in both the main and the validation cohort. We did not validate the associations of metabolites with time of day as all of our samples in the validation cohort were taken during the same time of the day.

Feature Attribution Analysis

We used SHAP (SHapley Additive exPlanations)³⁴, a recently introduced framework for interpreting predictions, which assigns each feature an importance value for a particular prediction. Briefly, for a specific prediction, a feature's SHAP value is defined as the change in the expected value of the model's output when this feature is observed vs when it is missing. It is computed using a sum that represents the impact of each feature being added to the model averaged over all possible orderings of features being introduced.

Individual SHAP values were computed for held-out subjects in 5-fold CV using the module TreeExplainer (version 0.24.0)³⁵′⁶⁶, based on models trained only on features from the respective feature group. Before training, we standardized the levels of target metabolites, so that SHAP values from different models would be comparable (they are measured in the same units as the target). In each CV fold we ran a random hyperparameter search consistent of 10 iterations using the module RandomizedSearchCV from sklearn (version 0.20.4), and chose the best model for predicting the held out subjects and computing SHAP values. In all feature attribution analyses we used the ungrouped list of 1170 metabolites.

For every feature, we computed the mean absolute SHAP value across all instances in a specific model, reflecting the mean impact of each feature on the predictions and serving as a feature importance measure. We further used these values to compute directional mean absolute SHAP values, by multiplying them with the sign of the Spearman correlation between the population feature and the target. Here, positive values indicate that higher feature values lead, on average, to higher predicted values, while negative values indicate that lower feature values lead, on average, to lower predicted values.

When performing feature attribution analysis with gut microbiome data as input, we only included the relative abundance of SGB representative genomes as features, taking only features which were present in over 5% of the samples, resulting with 753 bacterial taxa. When using diet as input, we only considered features which were present in at least 5% of the samples, resulting with 398 food types from logging and items from the FFQ.

Comparing Gradient Boosting Decision Trees with a Linear Model

We compared the EV of every single metabolite obtained for a GBDT and a Lasso regression model. The EV of all models were calculated in 5-fold CV, where in each fold we ran a hyperparameter search consistent of 10 iterations as described above. We used LightGBM as the GBDT model, and Lasso regression (sklearn, version 0.20.4) as the linear model, since its regularization scheme is better suited for a large number of features, as in the case of diet and gut microbiome composition. Since GBDT handles missing values well, we first imputed all missing values as the median of each feature to assure a fair comparison. When applying the models on the microbiome data, we used log 10 transformed values.

Estimating Relative Predictive Power of Feature Groups

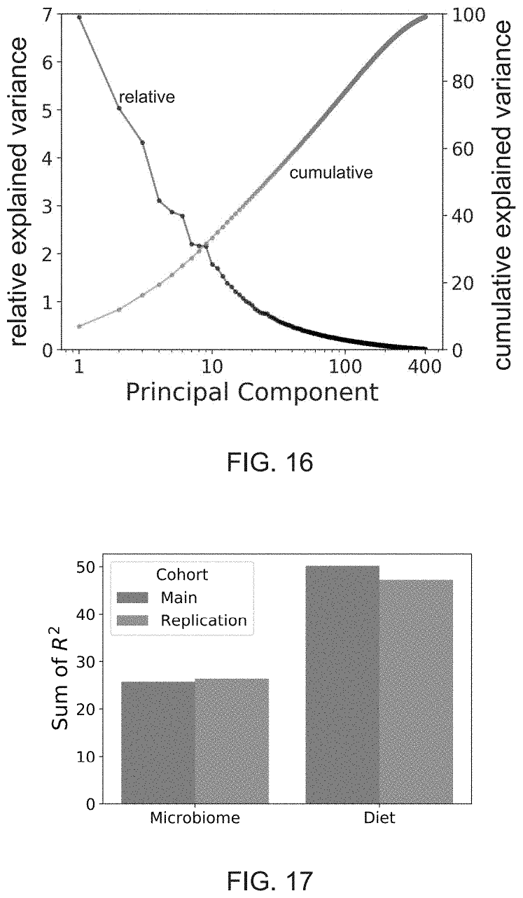

In order to estimate the relative predictive power of different feature groups we first applied a principal component analysis over the metabolite groups data to get the first 400 PCs which constitute >99% of the total variance in the data (FIG. 16). We then used 5-fold CV prediction models as described above to predict the PCs based on the different feature groups independently. As baseline, we used the full model, which consists of all features combined to predict the levels of the PCs, and estimated the overall fraction of variance explained by: (ΣᵢEVᵢ×PCᵢ)/(ΣᵢPCᵢ), where the summation is from i=1 to i=nPC, EVᵢ is the fraction of EV that the model recovers for PC i, PCᵢ is the fraction of variance that PC i explains out of the overall variation in the data, and nPC is the number of the first PCs, those which capture the most variation. For the features we have collected, we defined this sum obtained for the full model as the total explainable variance in circulating blood metabolites. Next, for every feature group we computed a similar expression and calculated the relative predictive power by dividing this expression by that of the full model. The estimates we present are for nPC=15, as the overall EV of the full model that we estimated using the first 15 PCs constitutes over 97% of the overall EV of the full model based on all 400 PCs.

Identification of Unknown Metabolites by Metabolon

Identification of unknown metabolites was done as previously described²⁹. Briefly, identification of tentative structural features for unknown biochemicals incorporates a detailed analysis of mass spec data, i.e., gathering information such as the accurate monoisotopic mass, the elution time and fragmentation pattern of the primary ion, and correlation to other molecules. The accurate monoisotopic mass is used to identify a likely structural formula for the unknown biochemical, which is then used to search against chemical structure databases. When a candidate structure fits the accurate monoisotopic mass and fragmentation data, an authentic standard is commercially purchased or synthesized (when possible). Conformation of a proposed structure is based on a match to three primary criteria, including co-elution with the unknown molecule of interest, and a high degree match to both the accurate monoisotopic mass and fragmentation pattern.

Interaction Networks

We used a graphical layout in order to visualize the associations of features with the levels of metabolites. The nodes are either metabolites or features, and the edges are the directional mean absolute SHAP values computed from models trained only on features from the respective feature group as described above. All networks were constructed using Cytoscape⁶⁷. The threshold for presenting SHAP values as edges was determined as 0.12, keeping the network sparse enough for convenience of visualization.

Analysis of Bread Intervention

In order to find the associations between metabolite levels and the consumption of both types of bread in the study cohort we computed the directional mean absolute SHAP values of the reported consumption of both white and whole-wheat bread for all metabolites. The SHAP values were computed in cross validation from models based only on the reported consumption of each type of bread. We ranked the metabolites according to their directional mean absolute SHAP value for each type of bread and used the top 5% positively and negatively driven metabolites for further analysis. The prediction models were constructed using 458 samples of distinct individuals, a subset of our cohort from which we excluded all samples of individuals which participated in the intervention study.

For each metabolite in every individual, we computed the FC of metabolite levels between the samples taken at the end of the first week of intervention and the start of that week. Prior to computing FC we imputed missing values with the minimum per metabolite and standardized their log (base 10) transformed levels. Furthermore, for each intervention group, we computed the mean FC of every metabolite based on the 10 samples from that group. We then compared the mean FC of the top 5% positively and negatively driven metabolites mentioned above within each intervention group by performing a rank sum test (Mann-Whitney U) over the mean FC.

For comparing the FC of betaine and cytosine between the two intervention groups, we used a Mann-Whitney U test.

LMM-Based Estimates of the Explained Variance of Metabolites Using Gut Microbiome

For the in-sample estimation of EV for metabolites based on gut microbiome we used a linear mixed model framework that we had recently developed³⁰. Briefly, we used GCTA⁶⁸, a tool used in statistical genetics for the estimating of SNP-based genetic kinship. Instead of a matrix of host SNPs, as is commonly used in GCTA, we used a kinship matrix computed over the presence-absence of microbial species which were also used as features in the out-of-sample prediction models. We added the storage time as a covariate to the model. P-values were computed using RL-SKAT⁶⁹.

### Results

Accurate and Reproducible Untargeted Serum Metabolomics from a Deeply Phenotyped Human Cohort

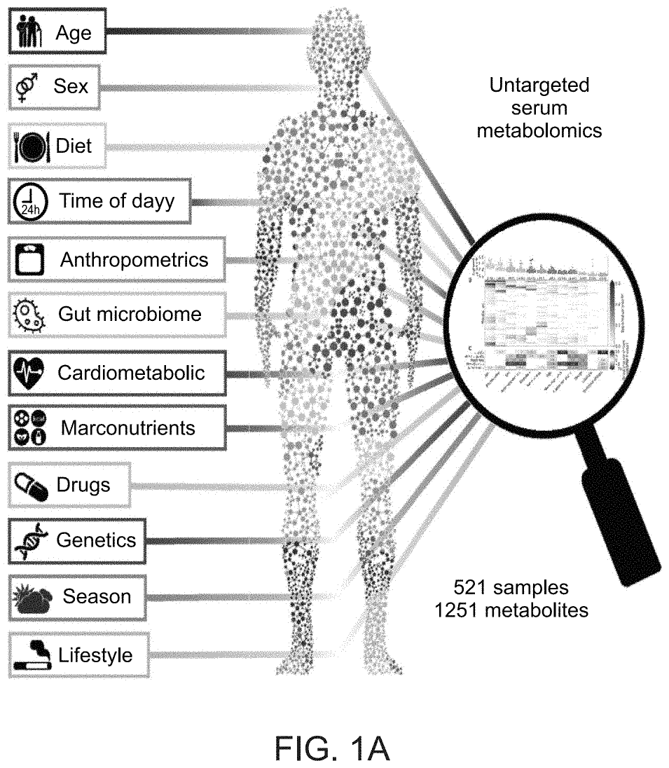

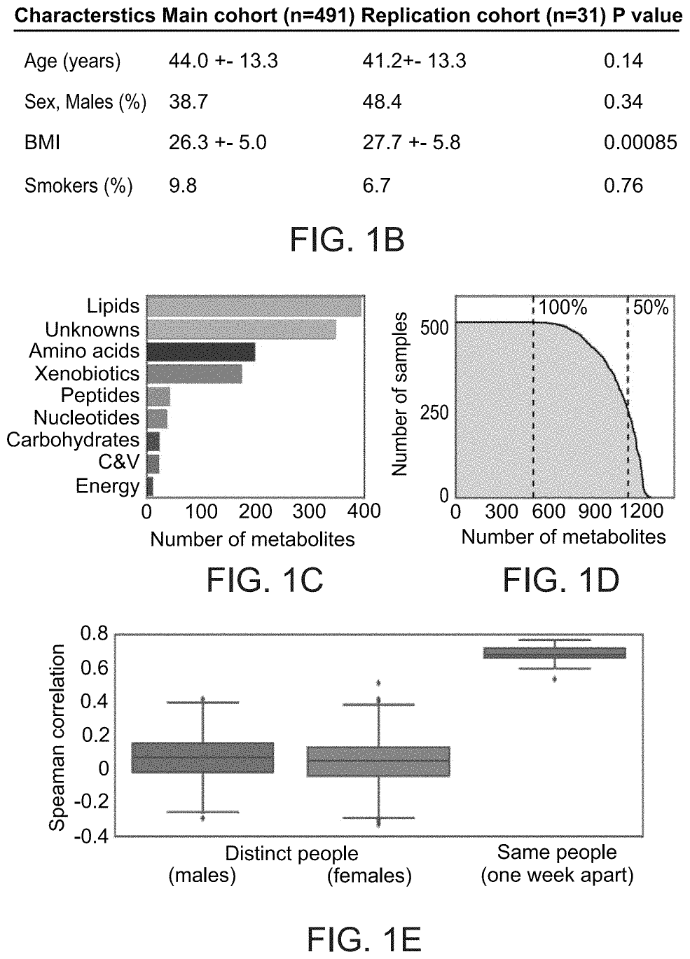

We used mass spectrometry to profile 521 serum samples from 491 healthy individuals for whom we previously collected extensive clinical data, anthropometrics measurements, cardiometabolic parameters, medication data, lifestyle, genetics, gut microbiome, dietary logging and answers to clinical and nutritional questionnaires²⁵ (FIG. 1A-B; Methods). Our untargeted metabolomics measured the levels of 1251 metabolites, covering a wide range of biochemicals including lipids, amino acids, xenobiotics, carbohydrates, peptides, nucleotides and approximately 30% unknown compounds (FIG. 1C, Methods). Most measured metabolites were prevalent across the cohort, including 498 metabolites detected in all samples, and 1104 metabolites detected in at least 50% of the samples (FIG. 1D).

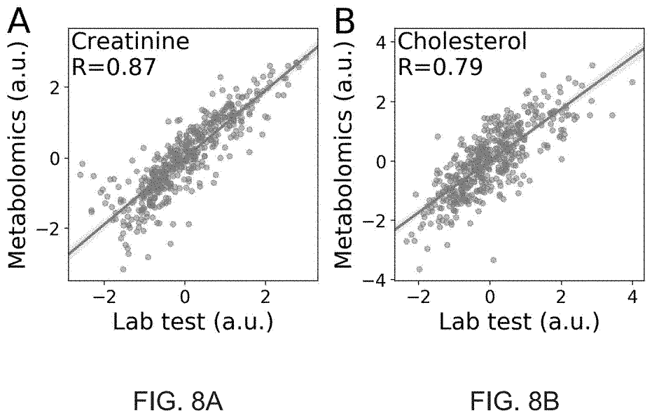

To test whether our measurements accurately report metabolite levels, we compared the metabolomic levels of creatinine and cholesterol to measurements of these compounds using standardized lab tests (Methods) performed separately on different blood samples taken from the same individual on a single visit, and found excellent agreement (R=0.87, creatinine; R=0.79, cholesterol, FIGS. 8A-B). Further demonstrating the reproducibility of our metabolomic measurements, we found that samples taken one week apart for 20 participants were significantly correlated (median Spearman R=0.68, std=0.06), in contrast to samples of different participants that show no correlation (median Spearman R=0.05, std=0.12; Methods; FIG. 1E). In addition to validating the reproducibility and accuracy of our data, these results are consistent with previous work showing that the human metabolic phenotype is stable even over several years²⁶, and suggest that this metabolic profile is a unique ‘fingerprint-like’ person-specific signature.

Diet, Microbiome, and Clinical Data Predict the Levels of Most Serum Metabolites

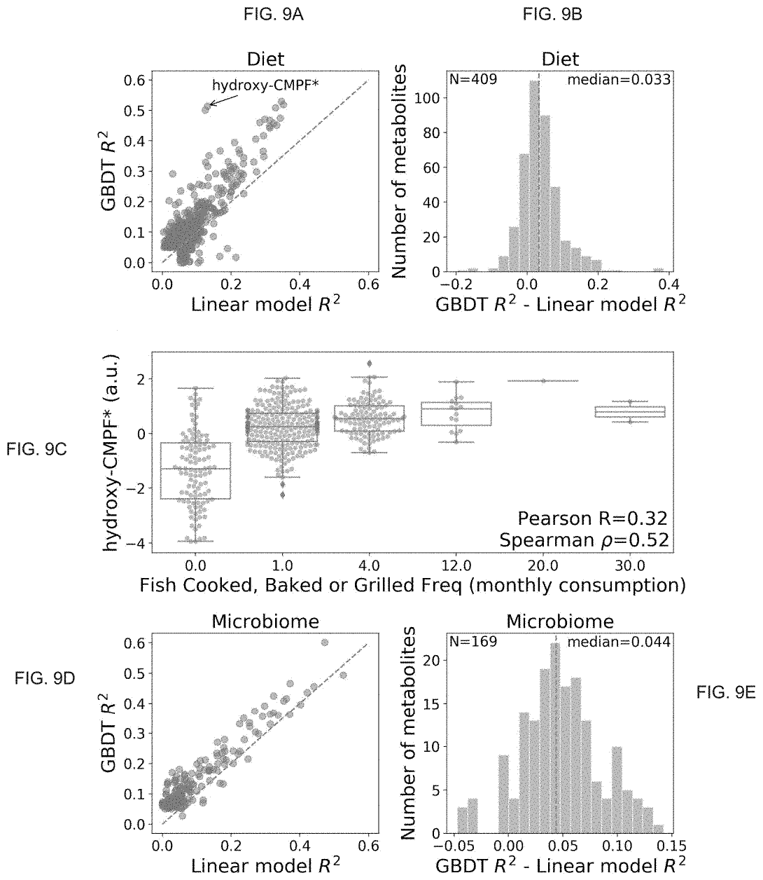

To estimate the extent to which metabolites can be predicted by the wealth of data we collected, we devised machine learning algorithms that predict the levels of each metabolite in held-out subjects (out-of-sample 5-fold cross validation prediction). One exception was human genetics, for which we considered the explained variance (EV) of each metabolite as that of the single most associated SNP (Methods). For prediction, we used gradient boosting decision trees²⁷ (GBDT; Methods) as these are powerful models which perform well in many different settings and can capture nonlinear interactions which are likely to be present in such a heterogeneous feature space and within the high dimensionality of the diet and microbiome data. We found that GBDT systematically outperformed linear models (Lasso; Methods), with a median and maximum EV gain of 3.3 and 38%, respectively, for prediction with diet data and 4.3 and 13% for prediction with microbiome data. (FIGS. 9A-E). Notably, our predictions were statistically significant for over 92% of the metabolite groups tested, following a strict Bonferroni correction (Methods), using at least one of the feature groups, with diet significantly explaining the largest number of metabolites (636), and gut microbiome explaining 389 metabolites (FIG. 2A-B). Together, our models explained over 10% of the variance for 467 metabolite groups (FIG. 2D), with a median R² of 10.7% (range 1.1-75.3%). For some metabolites, our models explained over 50% of the variance, using either genetics, sex, dietary, or microbiome features. For example, gut microbiome features alone explained 60% of the variance of the unknown compound X-16124.

To understand whether specific feature groups better predict certain types of metabolites, we checked, for each feature group, whether any type of metabolites was enriched with superior predictions (FIG. 2C). We found that clinical data, which includes age, sex, anthropometrics and cardiometabolic parameters, better predicted blood lipids, amino acids and peptides compared to xenobiotics and unknown compounds (FIG. 2C). In contrast, gut microbiome data predominantly explained levels of unknown compounds (p<0.005), highlighting the potential of the microbiome for discovering microbiome-derived metabolites and explaining the origin of the large number of unknown compounds.

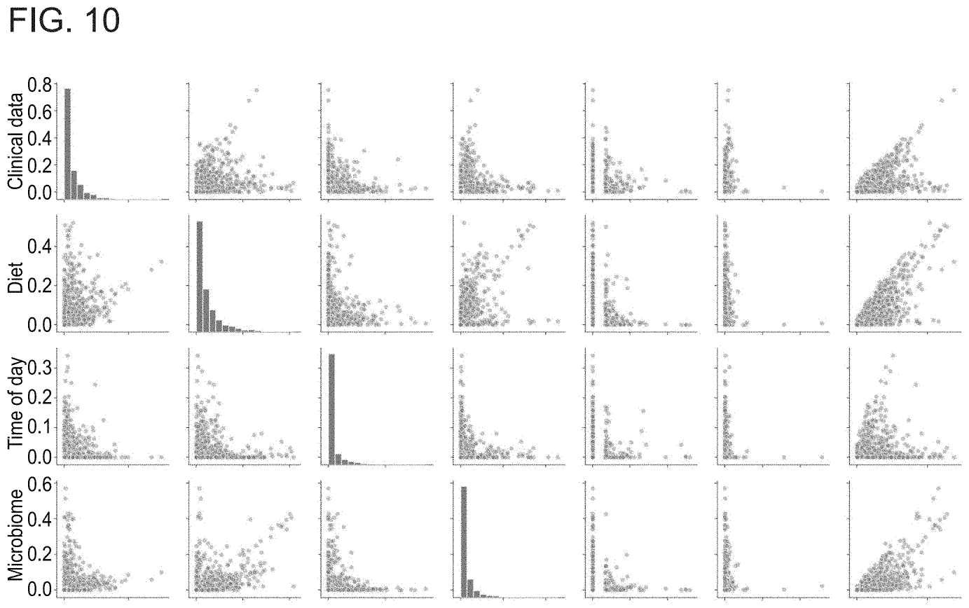

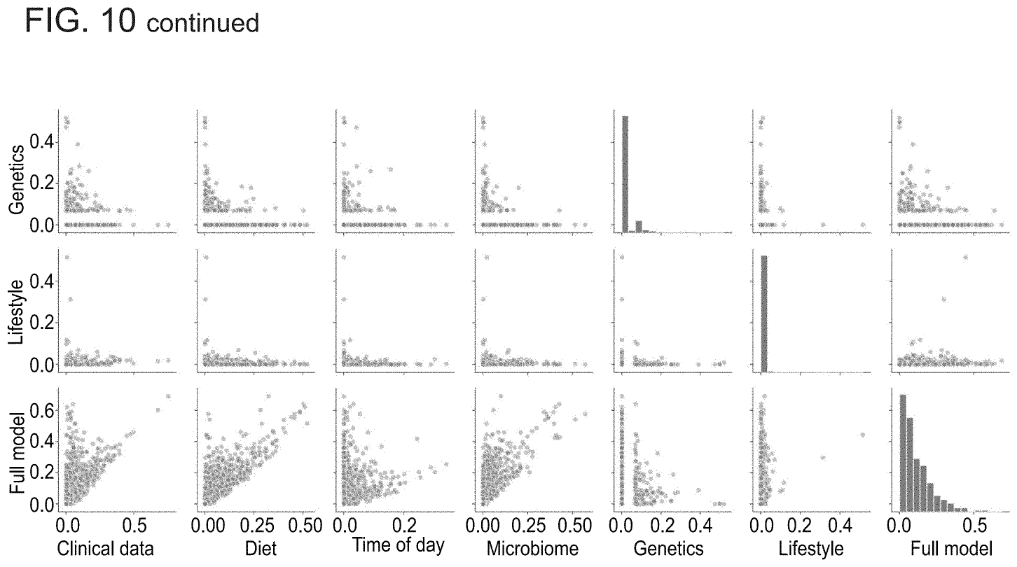

We next asked whether different feature groups predict metabolites with similar accuracy, by computing the correlation between the accuracy of metabolite predictions of every pair of input feature groups (FIG. 2E; FIG. 10). We found that predictions based on clinical data were significantly correlated with those of diet (Spearman R=0.32, p<10⁻²⁰), suggesting that some of the information captured by these feature groups is shared. A comparison to the lower (albeit significant) correlation between predictions made by clinical data and gut microbiome (R=0.22, p<10⁻¹²) implies that each capture unique information about metabolites. In addition, diurnal-based predictions were not correlated with any other feature group, demonstrating that metabolites explained by the time of the day were not predicted by and other data. Notably, predictions based on gut microbiome data had the highest correlation to predictions based on diet (R=0.44, p<10⁻²⁰), suggesting possible interactions between these feature groups in explaining the levels of many serum metabolites, an aspect that we further explore below. Finally, we found that the most genetically heritable metabolites could not be predicted by any of the other feature groups, as there was a negative correlation between the prediction accuracy of the full model and the heritability of metabolites (R=−0.14, p<10⁻⁵).

Taken together, our results show that we can devise statistically significant predictions for most serum metabolites using diet, gut microbiome, or other lifestyle and clinical parameters, with each feature group being especially informative with respect to a different set of metabolites. We next wished to estimate the general predictive power of each feature group across all measured serum metabolites. We built models predicting the principal components of the metabolomics data (FIG. 16), and then looked at the fraction of weighted explained variance in each feature group compared to that achieved with a model based on all features combined. We estimate that diet has the largest predictive power and could be used to infer 48.7% of the explainable variance in circulating blood metabolites compared to the full mode, while the prediction power of lifestyle factors constitute only 1.9% of that EV (FIG. 2F). Notably, gut microbiome data has 30.5% of the predictive power of the full model, and with a large portion of it not overlapping with the predictions of other data, this marks the importance of the microbiome in independently predicting and potentially determining serum metabolites levels.

Metabolite Predictions Replicate in an Independent Cohort

To test the robustness and reproducibility of our associations, we used the following approaches.

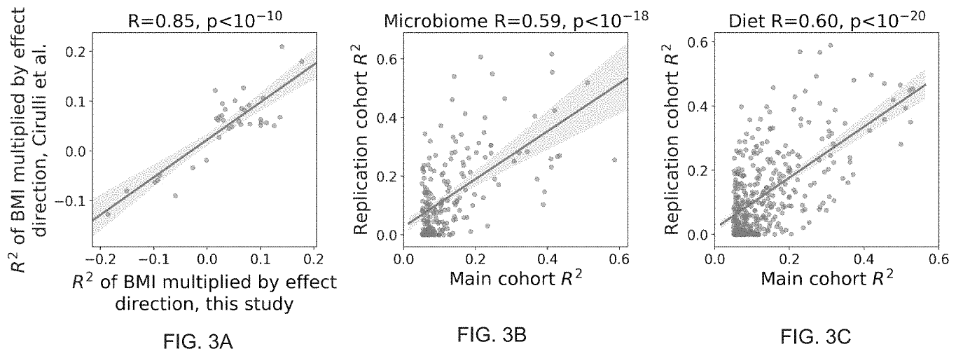

Firstly, we asked whether our cohort replicates significant associations between metabolite levels and body mass index (BMI) that were recently reported²⁸, and found that most of these associations replicated with high accuracy (Pearson R=0.85, p<10⁻¹⁰, FIG. 3A).

Secondly, we applied the same metabolomic profiling to an independent cohort of 31 individuals for which we also obtained identical measurements to those we had on the main cohort, including diet and gut microbiome data. Data from this additional cohort were not available to us while developing the prediction models. Notably, using our models, trained only on samples from our main cohort, for metabolites significantly predicted in our main cohort, we obtained predictions with similar accuracy on samples from this independent validation cohort. Specifically, for both diet and gut microbiome data, we found high agreement between the prediction accuracy and the overall predictive power of our models in the main cohort and in the replication cohort (Pearson R=0.59, p<10⁻¹⁸, microbiome; R=0.60, diet, p<10⁻²⁰; FIGS. 3B-C, FIG. 17). These results further validate that our models unravel robust associations between the levels of blood metabolites and the feature groups we measured.

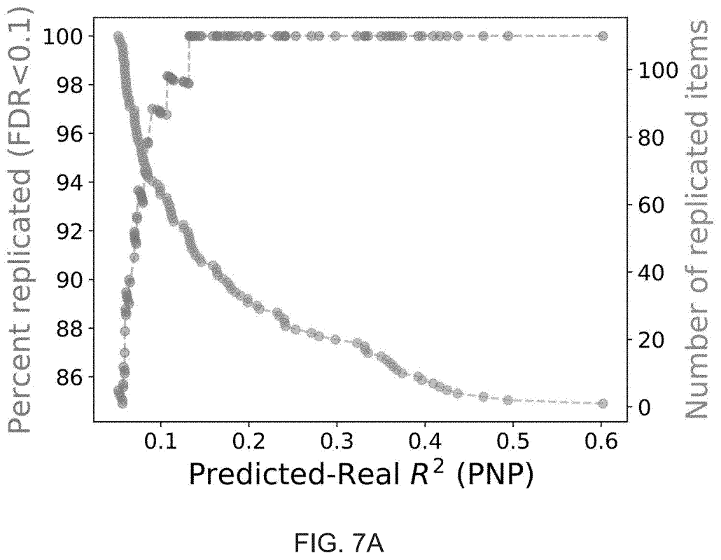

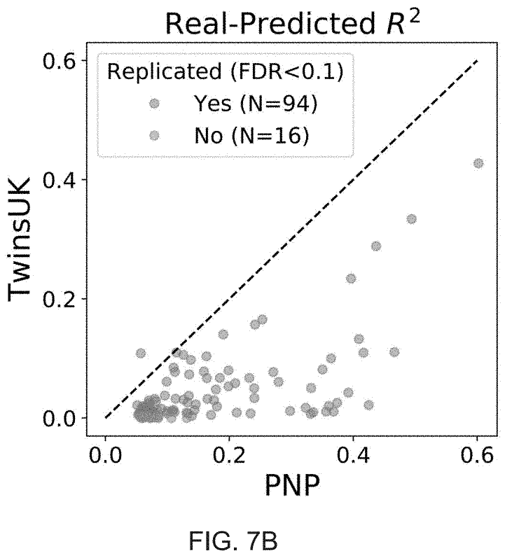

Thirdly, the model of the present embodiments was applied, without modification, to an independent cohort from the United Kingdom [UK Adult Twin Registry, www(dot)twinsuk(dot)ac(dot)uk]. FIGS. 7A and 7B demonstrate that at least the top 50 associations all replicate in this cohort, and that at least 94 out of the top 110 associations replicate. Table 6, below, summarizes the results for the top 110 metabolites, including the explained variance in the two cohorts, and the significance level of the replication, both raw and adjusted for multiple testing.

TABLE 6

| | | TwinsUK | TwinsUK | TwinsUK |
|---|---|---|---|---|
| | PNP R2 | p-value | q-value | R2 |
| X - 16124 | 0.60193 | 1.76E−123 | 1.94E−121 | 0.42746 |
| X - 11850 | 0.494078 | 1.30E−90 | 7.17E−89 | 0.334257 |
| 100000442 | 0.466141 | 2.06E−27 | 2.52E−26 | 0.110844 |
| X - 11843 | 0.436607 | 4.06E−76 | 1.49E−74 | 0.288504 |
| 100001405 | 0.424833 | 2.42E−06 | 5.31E−06 | 0.021954 |
| 100001315 | 0.416363 | 3.72E−27 | 3.72E−26 | 0.109801 |
| 100006191 | 0.4089 | 7.65E−33 | 1.05E−31 | 0.132607 |
| X - 12013 | 0.396407 | 4.51E−60 | 1.24E−58 | 0.234211 |
| 100001417 | 0.392058 | 3.90E−11 | 1.26E−10 | 0.042661 |
| 100001106 | 0.373802 | 3.05E−07 | 7.14E−07 | 0.025838 |
| 100001403 | 0.368044 | 0.000767 | 0.001223 | 0.011239 |
| X - 12816 | 0.363962 | 7.36E−25 | 5.40E−24 | 0.100435 |
| 100001400 | 0.360403 | 4.89E−06 | 1.03E−05 | 0.020633 |
| 100001399 | 0.355817 | 0.00088 | 0.001383 | 0.010987 |
| X - 21442 | 0.350055 | 2.75E−20 | 1.68E−19 | 0.081518 |
| 849 | 0.335179 | 0.001585 | 0.002357 | 0.009912 |
| 100000011 | 0.331918 | 5.65E−13 | 2.00E−12 | 0.050565 |
| 100000437 | 0.331126 | 0.00761 | 0.010085 | 0.007087 |
| 100000453 | 0.322879 | 2.85E−05 | 5.43E−05 | 0.017334 |
| 100001397 | 0.297921 | 0.000428 | 0.000736 | 0.01231 |
| 100006098 | 0.279406 | 2.39E−15 | 9.38E−15 | 0.060702 |
| X - 12216 | 0.270791 | 2.52E−19 | 1.26E−18 | 0.077493 |
| 100000010 | 0.253025 | 2.73E−41 | 6.00E−40 | 0.165457 |
| 100002253 | 0.241426 | 3.84E−39 | 7.03E−38 | 0.15722 |
| X - 23649 | 0.24028 | 4.83E−09 | 1.44E−08 | 0.033625 |
| 100004112 | 0.240092 | 7.02E−13 | 2.41E−12 | 0.050159 |
| X - 23997 | 0.234264 | 0.005032 | 0.00675 | 0.007825 |
| 100001092 | 0.232021 | 6.21E−17 | 2.84E−16 | 0.067422 |
| 100001402 | 0.211918 | 0.002301 | 0.003331 | 0.009235 |
| 100001657 | 0.209332 | 6.99E−15 | 2.65E−14 | 0.058715 |
| X - 12230 | 0.198428 | 1.34E−13 | 4.90E−13 | 0.053246 |
| 100004111 | 0.198406 | 5.71E−20 | 3.31E−19 | 0.080192 |
| 100002021 | 0.190037 | 7.69E−35 | 1.21E−33 | 0.140485 |
| 100001083 | 0.184587 | 6.50E−17 | 2.84E−16 | 0.067341 |
| X - 12329 | 0.179864 | 9.28E−06 | 1.89E−05 | 0.019433 |
| X - 12306 | 0.178041 | 2.19E−12 | 7.29E−12 | 0.048042 |
| X - 21821 | 0.175098 | 3.16E−08 | 8.27E−08 | 0.0301 |
| X - 23639 | 0.170295 | 0.017756 | 0.022195 | 0.005596 |
| X - 17351 | 0.164692 | 1.09E−08 | 2.99E−08 | 0.032101 |
| 100002911 | 0.163574 | 6.72E−17 | 2.84E−16 | 0.067279 |
| 100001658 | 0.162903 | 1.01E−25 | 7.97E−25 | 0.103956 |
| 100000014 | 0.158807 | 1.50E−19 | 8.25E−19 | 0.078438 |
| X - 11315 | 0.145696 | 1.33E−06 | 3.05E−06 | 0.023072 |
| 100001086 | 0.143621 | 0.000207 | 0.000361 | 0.013655 |
| 100009002 | 0.139306 | 0.051746 | 0.061205 | 0.003771 |
| X - 21752 | 0.13783 | 3.48E−24 | 2.40E−23 | 0.097664 |
| 1135 | 0.135034 | 2.24E−18 | 1.07E−17 | 0.073506 |
| 100000467 | 0.134245 | 6.24E−10 | 1.91E−09 | 0.037467 |
| X - 12730 | 0.132732 | 0.004609 | 0.006337 | 0.007983 |
| X - 17185 | 0.13236 | 2.45E−07 | 5.99E−07 | 0.026249 |
| 100000580 | 0.131515 | 0.877499 | 0.877499 | 2.37E−05 |
| X - 22162 | 0.130587 | 0.00256 | 0.003658 | 0.009042 |
| X - 21286 | 0.125927 | 1.78E−08 | 4.79E−08 | 0.031173 |
| X - 17145 | 0.125471 | 2.54E−26 | 2.15E−25 | 0.106407 |
| 100001148 | 0.114388 | 2.84E−27 | 3.13E−26 | 0.110276 |
| 100000436 | 0.112946 | 8.08E−09 | 2.34E−08 | 0.03266 |
| 100001510 | 0.111889 | 2.40E−19 | 1.26E−18 | 0.077585 |
| 100000447 | 0.111378 | 0.001518 | 0.002287 | 0.009992 |
| 136 | 0.109922 | 4.15E−21 | 2.68E−20 | 0.084944 |
| 100005864 | 0.109533 | 0.000188 | 0.000334 | 0.013825 |
| X - 12738 | 0.107436 | 0.00053 | 0.000861 | 0.011917 |
| 1258 | 0.106308 | 0.858411 | 0.866286 | 3.18E-05 |
| X - 21339 | 0.099392 | 0.004846 | 0.006581 | 0.007893 |
| 100004208 | 0.098996 | 0.000519 | 0.000861 | 0.011955 |
| 100003434 | 0.098884 | 1.78E−15 | 7.26E−15 | 0.061243 |
| 339 | 0.09559 | 5.17E−10 | 1.62E−09 | 0.037821 |
| X - 11880 | 0.090108 | 6.26E−05 | 0.000117 | 0.015872 |
| 100001456 | 0.085311 | 0.688289 | 0.714263 | 0.000161 |
| X - 11308 | 0.084777 | 0.052811 | 0.0618 | 0.003737 |
| 100004046 | 0.0844 | 0.110712 | 0.126858 | 0.002537 |
| X - 18914 | 0.082799 | 0.000172 | 0.00031 | 0.013996 |
| 100002154 | 0.081625 | 6.56E−08 | 1.64E−07 | 0.028726 |
| X - 13835 | 0.079674 | 0.317852 | 0.339453 | 0.000996 |
| 100001624 | 0.079165 | 9.24E−09 | 2.61E−08 | 0.032409 |
| 100002241 | 0.078238 | 0.046573 | 0.055685 | 0.003947 |
| 100001022 | 0.077824 | 0.012876 | 0.016664 | 0.006158 |
| X - 11372 | 0.077439 | 0.001173 | 0.001791 | 0.010462 |
| X - 21736 | 0.07603 | 0.000917 | 0.001421 | 0.010912 |
| X - 11381 | 0.074129 | 0.000467 | 0.000791 | 0.012149 |
| 381 | 0.072941 | 0.755927 | 0.769926 | 9.65E-05 |
| 100000445 | 0.07275 | 0.014756 | 0.018874 | 0.005919 |
| 100001162 | 0.072115 | 0.559964 | 0.586629 | 0.000339 |
| 100001743 | 0.071197 | 0.002787 | 0.003931 | 0.008889 |
| 100004110 | 0.070462 | 1.67E−06 | 3.74E−06 | 0.02265 |
| 1668 | 0.070161 | 2.86E−07 | 6.83E−07 | 0.025962 |
| 100001756 | 0.070086 | 1.44E−05 | 2.88E−05 | 0.01861 |
| X - 23587 | 0.069924 | 3.78E−08 | 9.67E−08 | 0.029761 |
| 1518 | 0.069794 | 0.176014 | 0.197566 | 0.001827 |
| 100003001 | 0.064862 | 0.096912 | 0.112213 | 0.002748 |
| X - 12221 | 0.063781 | 2.86E−05 | 5.43E−05 | 0.01733 |
| 100001126 | 0.063769 | 0.305695 | 0.329671 | 0.001047 |
| 100002122 | 0.062773 | 1.69E−05 | 3.32E−05 | 0.018315 |
| 100008999 | 0.061545 | 0.025154 | 0.031089 | 0.004993 |
| 100001300 | 0.061295 | 0.041996 | 0.050764 | 0.004121 |
| 100001605 | 0.060642 | 0.000532 | 0.000861 | 0.01191 |
| 100001051 | 0.060427 | 0.199497 | 0.221663 | 0.001642 |
| 100006126 | 0.060353 | 0.027193 | 0.033235 | 0.004859 |
| X - 16935 | 0.059659 | 6.37E−06 | 1.32E−05 | 0.020139 |
| 100004328 | 0.059032 | 0.747708 | 0.768672 | 0.000103 |
| 100008920 | 0.058966 | 0.516784 | 0.546598 | 0.00042 |
| 100000042 | 0.058828 | 0.258925 | 0.281997 | 0.001272 |
| 100000841 | 0.058762 | 0.002027 | 0.002974 | 0.009465 |
| X - 12822 | 0.057473 | 0.000131 | 0.00024 | 0.014499 |
| X - 23314 | 0.057217 | 0.204197 | 0.224617 | 0.001608 |
| X - 15728 | 0.057172 | 7.08E−27 | 6.49E−26 | 0.108667 |
| 100001541 | 0.056209 | 0.131519 | 0.149145 | 0.002269 |
| 100001055 | 0.056172 | 0.011833 | 0.015495 | 0.006306 |
| X - 18249 | 0.054302 | 0.00363 | 0.005054 | 0.008412 |
| 240 | 0.052766 | 0.017548 | 0.022187 | 0.005616 |
| 100001034 | 0.051379 | 2.98E−06 | 6.43E−06 | 0.021559 |

Novel Associations Between Human Genetics and Circulating Blood Metabolites

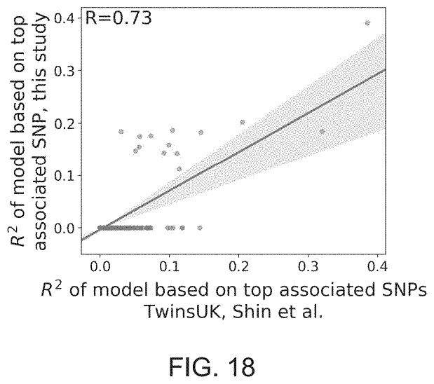

Several studies found that human genetics affect serum metabolites^{6,7,29}. In this study we measured hundreds of novel molecules which were not yet identified in previously published studies including both serum metabolomics and human genetics, and therefore set to look for novel associations between single nucleotide polymorphisms (SNPs) and serum metabolites levels. Notably, we found 553 statistically significant associations with genetic for 67 metabolites (p<5×10⁻¹¹), many of which are novel. This includes the unknown metabolite X-24809 which was associated with rs4539242 that alone explained 52% of its variance. To further validate our results, we set to replicate previous reported associations between SNPs and the levels of circulating blood metabolites. Among the 529 metabolites analysed in a previous large study which included 7824 individuals⁶, 301 were also measured by us using the same MS platform (Metabolon, inc.; Methods), and 111 of them were reported to have significant associations with SNPs. Due to the difference in cohort sizes, we were limited in terms of the statistical power needed for the replication of relatively small effect variants. Overall, we found a high correlation between the EV of a model based on top significantly associated SNPs in the previous study and a model based on the single top associated SNP in our study (Pearson R=0.73, p<10⁻²⁰; FIG. 18). In our cohort, we found significant associations between SNPs and 14 out of the 111 metabolites, but no significant associations for any of the remaining 190 metabolites (p<10⁻⁶ for only replicating a subgroup of known associations, Fisher exact test). We found that in 11 cases out of the 14 the association between the metabolite and the specific SNP reported in the previous study was replicated in this study, while in the other three cases the associations that we found are novel, in all these cases, the EV by the reported SNP in both the previous study and in this study was highly similar (R=0.91, p<10⁻⁴).

Diet and Gut Microbiome Data Independently Explain a Wide Range of Metabolites

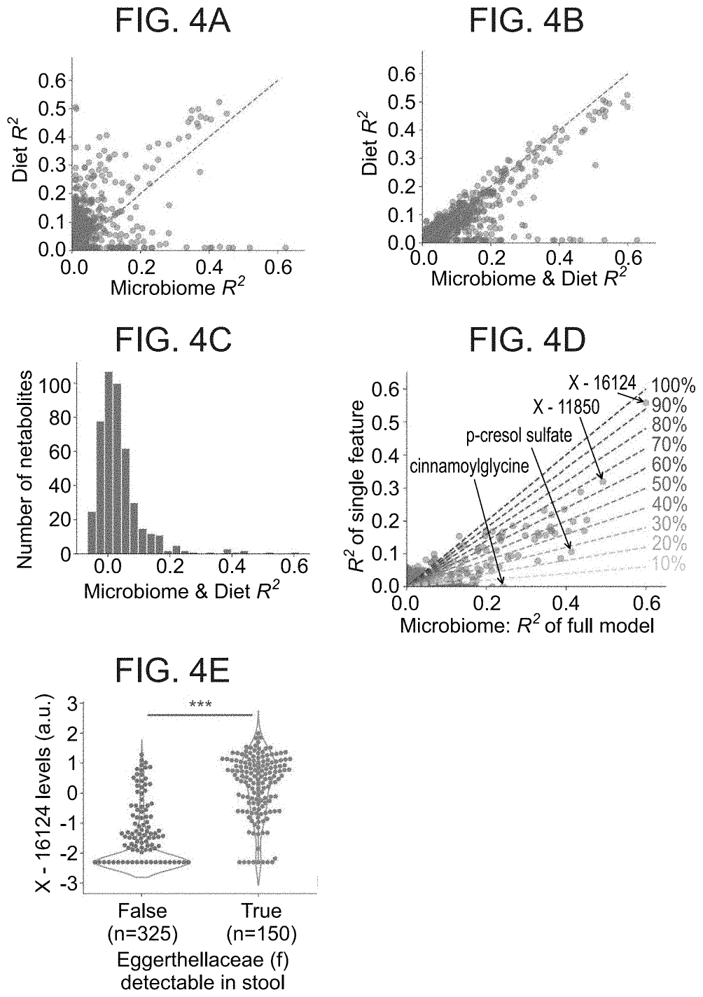

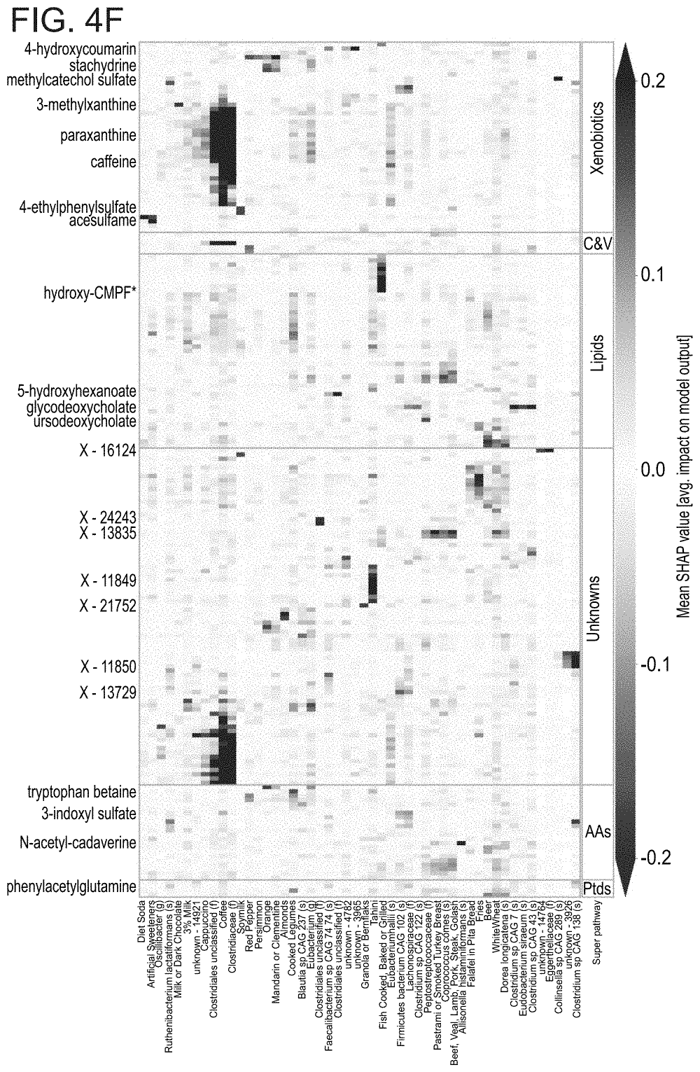

Diet and gut microbiome had the largest predictive power and there is a significant correlation in the metabolites that they each predicted well (FIG. 2E). Since diet is known to modulate the composition of the gut microbiome³⁰⁻³², we sought to unravel which metabolites are more likely to be driven by diet and which by the gut microbiota, by comparing the EV of metabolites obtained by a model based on diet and by one based on gut microbiome data (FIG. 4A). If the prediction of metabolites by the microbiome was confounded by diet, in other words if diet affects both the metabolites and the microbiome, then we would expect that all microbiome-predicted metabolites could also be predicted (possibly with higher accuracy) by diet. However, we found that although some metabolites were significantly predicted by both diet and gut microbiome, many metabolites were predicted well by only one of the two data types (FIG. 4A). To measure the contribution of the microbiome to the prediction of each metabolite, we compared the EV of a model based on both diet and microbiome to a model based only on diet data (FIG. 4B). We found that adding microbiome data to the prediction model improved the model's accuracy in 66% of cases (median and max gain of 2.1%, 61.2% respectively; FIG. 4C). Finally, 34 metabolites were significantly predicted only using the gut microbiome, and the predictions of multiple others improved upon introducing microbiome to the models. Taken together, these results suggest that the gut microbiome modulates the production of many circulating metabolites independent of diet.

We next sought to interpret the diet and gut microbiome models and ask which dietary features and bacterial taxa drive the predictions of each metabolite. Our diet data consists of both answers to food frequency questionnaires and one week of dietary logging collected in real-time via a mobile App we devised²⁵, and thus allows us to address the predictive power of both long term and short term nutritional patterns. The gut microbiome composition is represented as relative abundance of bacterial species and we estimated it based on high depth metagenomic sequencing followed by mapping to a unique and comprehensive microbial database that was recently published³³ (Methods). In order to explain the output of our machine learning models and find specific associations between features and metabolite levels we used SHAP (SHapley Additive exPlanations)³⁴, a feature attribution analysis tool which assigns each feature an importance value (SHAP value) for a particular prediction³⁵ (Methods). Shapley values based analysis in gut microbiome data was recently demonstrated to be useful, as it allowed for the estimation of complex contributions of gut microbiome taxa to functional shifts, while maintaining global community composition properties³⁶.

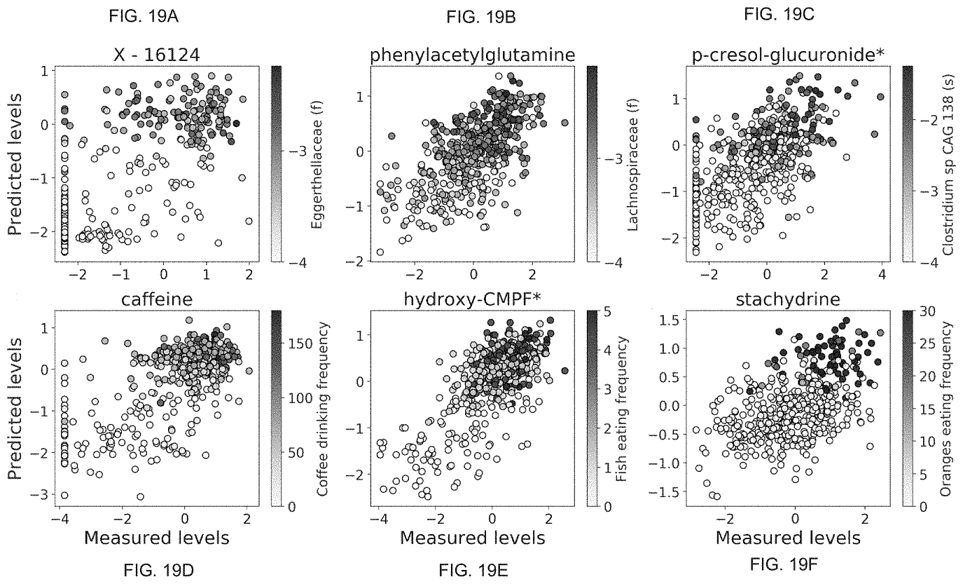

We found dozens of diet features and bacterial taxa that were strongly predictive of blood metabolites in our models (FIG. 4F; FIGS. 19A-F). Notably, the reported consumption of coffee (both long- and short-term) had higher importance compared to other dietary features with respect to a large number of xenobiotics and unknown compounds. As previously reported³⁷, metabolites from the xanthine metabolism pathway such as paraxanthine (Prediction Pearson R=0.64, p<10⁻²⁰, based on diet data) and caffeine (Prediction R=0.68, p<10⁻²⁰) were significantly predicted using coffee consumption. These metabolites were also significantly predicted using gut microbiome data, with one bacterial feature from the Clostridiceae family being the main predictor. Another strong predictor was the reported consumption of fish, which was assigned with the highest SHAP values in models based on diet features which accurately predicted the levels of several blood lipids such as 3-Carboxy-4-methyl-5-propyl-2-furanpropionic acid (CMPF; prediction R=0.71, p<10⁻²⁰), a potent uremic toxin known to accumulate in the serum of chronic kidney disease (CKD) patients³⁸ and which was also suggested to prevent and reverses steatosis³⁹. Other examples included saccharin (Prediction R=0.6, p<10⁻²⁰) and acesulfame (Prediction R=0.47, p<10⁻²⁰, two artificial sweeteners whose main predictors were the reported consumption of artificial sweeteners and diet soda. As mentioned above, microbiome data alone accurately predicted the levels of many metabolites such as X-16124 (Pearson R=0.77, p<10⁻²⁰), an unknown metabolite whose main predictor is the relative abundance of a bacteria from the Eggerthellaceae family, and X-11850 (R=0.7, p<10⁻²⁰), another unknown compound whose main predictor is a species of Clostridium. The microbiome data was also highly predictive of two uremic toxins (phenylacetylglutamine, R=0.63, p<10⁻²⁰, and indoxyl sulfate, R=0.37, p<10⁻²⁰) previously reported in association with CKD⁴⁰ and several other comorbidities^{41,42}, and these predictions were positively driven by a bacteria from the Lachnospiraceae family.

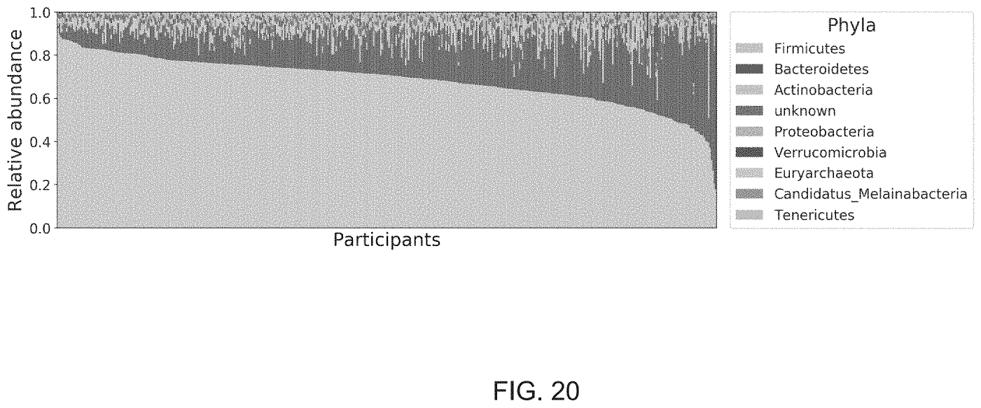

As a more global view, we next asked whether a few bacterial features are important for the prediction of many metabolites, or whether metabolite prediction is specific to several unique important taxa. To this end, for each metabolite we defined its main predictor as the bacterial taxa with the maximal mean absolute SHAP value. We found that 19 bacterial taxa were the main predictors for the top 50 predicted metabolites (Prediction R>0.4; Table 7). One bacterial feature from the Clostridiceae family was the main predictor of 22 of these metabolites which are also strongly associated with coffee consumption in diet-based models. Clostridium sp. CAG:138 was the main predictor of 5 metabolites, including 3 unknown compounds, phenylacetylcarnitine (R=0.47, p<10⁻²⁰) and p-cresol-glucuronide (R=0.64, p<10⁻²⁰) which was previously reported to be metabolized by Clostridium⁴³. Furthermore, 6 bacterial features were the main predictors of 2 metabolites each, and each of the other 11 bacterial features was a main predictor of a single metabolite. Hence, in most cases many specific bacteria are required in order to accurately predict the levels of distinct metabolites, but in some cases a single bacteria might underlie the predictions of a broad metabolic pathway involving dozens of metabolites. In terms of higher bacterial taxonomy levels, among the bacterial features that best predicted the top 100 metabolites, 89 belonged to Firmicutes, 4 to Actinobacteria and 7 to an unknown phylum, showing the strong predictive power of Firmicutes. Interestingly, although Bacteroidetes is the second most abundant phylum in our cohort (FIG. 20), none of its species was a main predictor for any of the 100 metabolites best predicted with microbiome data.

We next asked whether these single best predictors are sufficient for the accurate prediction of each metabolite or whether additional information regarding the composition of the gut microbiome is needed. To this end, for each metabolite we compared the results from a full model of the microbiome to a prediction model based only on the strongest predictor (FIG. 4D). We found that for most of the metabolites which were best predicted using microbiome data, a model based only on the single best predictor could explain 20-70% of the variance that the full model explained with a median of 36%, showing that for many metabolites the relative abundance of other bacterial taxa are needed for better predictions. In addition, this result implies that the levels of these metabolites are associated with different bacterial taxa in different individuals, as in the case of cinnamoylglycine which is significantly predicted using the full gut microbiome composition (R=0.49, p<10⁻²⁰), yet a model based only on its top predictor fails to provide a significant prediction. In contrast, some metabolites are exclusively predicted by a single bacterial species, as in the case of the unknown metabolite X-16124, for which a model based only on the relative abundance of a bacteria from the Eggerthellaceae family explained 93% of the variance compared to the full model. Indeed in 95% of the individuals where this bacteria was detectable in stool this metabolite was also detectable in their serum, compared to only 23% of individuals for which this bacteria was not detected in their stool (p<10⁻²⁰, FIG. 4E).

TABLE 7

| | | Prediction | mean absolute |
|---|---|---|---|
| BIOCHEMICAL | Main driver | Pearson R | SHAP values |
| X - 11850 | (14306) S: Clostridium sp | 0.71031646 | 0.377656597 |
| | CAG 138 | | |
| X - 11843 | (14306) S: Clostridium sp | 0.666618163 | 0.354302695 |
| | CAG 138 | | |
| X - 12013 | (14306) S: Clostridium sp | 0.648938368 | 0.302711426 |
| | CAG 138 | | |
| p-cresol-glucuronide* | (14306) S: Clostridium sp | 0.634978874 | 0.169905629 |
| | CAG 138 | | |
| phenylacetylcarnitine | (14306) S: Clostridium sp | 0.452402753 | 0.102290219 |
| | CAG 138 | | |
| 5alpha-androstan- | (14311) F: Clostridiaceae | 0.43740413 | 0.099953342 |
| 3beta,17alpha-diol disulfate | | | |
| 4-methylcatechol sulfate | (14397) S: Collinsella sp | 0.403773094 | 0.210294783 |
| | CAG 289 | | |
| X - 16124 | (14816) F: Eggerthellaceae | 0.797710646 | 0.731921094 |
| 4-ethylcatechol sulfate | (14861) U: Unknown | 0.413293556 | 0.13518011 |
| X - 12816 | (14921) U: Unknown | 0.557555233 | 0.345160261 |
| X - 24410 | (15119) F: Clostridiales | 0.444238234 | 0.208132929 |
| | unclassified | | |
| X - 24811 | (15154) F: Clostridiales | 0.538397579 | 0.486675273 |
| | unclassified | | |
| 5-acetylamino-6-amino-3- | (15154) F: Clostridiales | 0.525783812 | 0.384463759 |
| methyluracil | unclassified | | |
| caffeine | (15154) F: Clostridiales | 0.479015705 | 0.247431918 |
| | unclassified | | |
| 1,7-dimethylurate | (15154) F: Clostridiales | 0.516271766 | 0.379716336 |
| | unclassified | | |
| 1,3-dimethylurate | (15154) F: Clostridiales | 0.506154168 | 0.432380221 |
| | unclassified | | |
| theophylline | (15154) F: Clostridiales | 0.500430564 | 0.35139537 |
| | unclassified | | |
| paraxanthine | (15154) F: Clostridiales | 0.494814811 | 0.480019756 |
| | unclassified | | |
| quinate | (15154) F: Clostridiales | 0.550659433 | 0.320069825 |
| | unclassified | | |
| X - 21442 | (15154) F: Clostridiales | 0.485910453 | 0.325317846 |
| | unclassified | | |
| 1,3,7-trimethylurate | (15154) F: Clostridiales | 0.481535209 | 0.332818145 |
| | unclassified | | |
| 1-methylurate | (15154) F: Clostridiales | 0.543233686 | 0.354953786 |
| | unclassified | | |
| 1-methylxanthine | (15154) F: Clostridiales | 0.522307846 | 0.409986488 |
| | unclassified | | |
| citraconate/glutaconate | (15154) F: Clostridiales | 0.397920928 | 0.126892778 |
| | unclassified | | |
| X - 23649 | (15154) F: Clostridiales | 0.405318367 | 0.214755421 |
| | unclassified | | |
| X - 12837 | (15154) F: Clostridiales | 0.449837283 | 0.17833279 |
| | unclassified | | |
| 3-methyl catechol sulfate (1) | (15154) F: Clostridiales | 0.430459047 | 0.195565418 |
| | unclassified | | |
| X - 23655 | (15154) F: Clostridiales | 0.419593407 | 0.266598091 |
| | unclassified | | |
| 3-hydroxypyridine sulfate | (15154) F: Clostridiales | 0.421956386 | 0.158726692 |
| | unclassified | | |
| taurolithocholate 3-sulfate | (15216) F: Clostridiales | 0.409120959 | 0.082624734 |
| | unclassified | | |
| 3-phenylpropionate | (15236) G: Firmicutes | 0.463566191 | 0.061088396 |
| (hydrocinnamate) | unclassified | | |
| cinnamoylglycine | (15236) G: Firmicutes | 0.50723076 | 0.095710259 |
| | unclassified | | |
| isoursodeoxycholate | (15265) S: Firmicutes | 0.45029973 | 0.072828701 |
| | bacterium CAG 103 | | |
| p-cresol sulfate | (15271) S: Ruthenibacterium | 0.588586011 | 0.131445263 |
| | lactatiformans | | |
| X - 23997 | (15356) U: Unknown | 0.413579828 | 0.109760662 |
| X - 12216 | (15369) S: Faecalibacterium | 0.473979701 | 0.081022475 |
| | sp CAG 74 | | |
| X - 12126 | (15369) S: Faecalibacterium | 0.506863696 | 0.116282931 |
| | sp CAG 74 | | |
| X - 12261 | (3926) U: Unknown | 0.652153347 | 0.165363667 |
| X - 17612 | (3957) F: Lachnospiraceae | 0.426420399 | 0.086057928 |
| phenylacetate | (3957) F: Lachnospiraceae | 0.564932682 | 0.083074571 |
| X - 17469 | (4552) S: Ruminococcus sp | 0.405437752 | 0.067777374 |
| glycolithocholate sulfate* | (4552) S: Ruminococcus sp | 0.458290469 | 0.113144654 |
| X - 21821 | (4564) S: Ruminococcus | 0.433421509 | 0.067984888 |
| | torques | | |
| X - 17351 | (4564) S: Ruminococcus | 0.416500421 | 0.085811771 |
| | torques | | |
| X - 12851 | (4782) U: Unknown | 0.479290527 | 0.141919097 |
| indolepropionate | (4810) S: Blautia sp CAG | 0.402571341 | 0.090296832 |
| | 237 | | |
| phenylacetylglutamine | (4951) S: Roseburia | 0.605077279 | 0.072142918 |
| | intestinalis | | |
| X - 13729 | (5190) S: Firmicutes | 0.412316821 | 0.14903645 |
| | bacterium CAG 102 | | |
| N-acetyl-cadaverine | (5843) S: Allisonella | 0.464233346 | 0.339842275 |
| | histaminiformans | | |
| ursodeoxycholate | (6148) F: | 0.412223334 | 0.133438158 |
| | Peptostreptococcaceae | | |

We also explored which metabolites were best explained by gut microbiome data. For each of the metabolite groups which were significantly predicted using the gut microbiome we computed a score between 0 and 1, representing the fraction of variance that the microbiome data model explains out of that explained by the sum of the microbiome model and the next best model from the feature groups except microbiome. For 80 microbiome predicted metabolite groups, the score was higher than 0.5, indicating that microbiome had the highest predictive power among all feature groups tested (Table 8).

TABLE 8

| | | | Next best r2 | Next best |
|---|---|---|---|---|
| | | | (other than | (other than |
| BIOCHEMICAL | Score | Microbiome r2 | Microbiome) | Microbiome) |
| carnitine | 0.276977 | 0.059446 | 0.155178 | Sex |
| 3-phenylpropionate | 0.765705 | 0.212152 | 0.064916 | Diet |
| (hydrocinnamate) | | | | |
| phenylacetate + | 0.927233 | 0.376409 | 0.02954 | Diet |
| phenylacetylglutamine | | | | |
| hippurate | 0.435493 | 0.132882 | 0.172249 | Diet |
| xanthurenate | 0.342061 | 0.016314 | 0.031379 | Diet |
| 3-methyl-2-oxovalerate + 4- | 0.157003 | 0.032823 | 0.176237 | Diet |
| methyl-2-oxopentanoate | | | | |
| 3-methylhistidine | 0.237835 | 0.098778 | 0.316545 | Diet |
| glucuronate | 0.308446 | 0.026981 | 0.060492 | Time of day |
| glycodeoxycholate | 0.7384 | 0.150068 | 0.053166 | Time of day |
| quinate | 0.367987 | 0.303854 | 0.521864 | Diet |
| theobromine + 7- | 0.276803 | 0.044924 | 0.117371 | Diet |
| methylxanthine | | | | |
| gentisate | 0.365171 | 0.104458 | 0.181595 | Diet |
| paraxanthine | 0.366152 | 0.24232 | 0.419483 | Diet |
| indolelactate | 0.090336 | 0.023379 | 0.235425 | Sex |
| 3-indoxyl sulfate | 0.756091 | 0.129252 | 0.041696 | Diet |
| 1,5-anhydroglucitol (1,5-AG) | 0.376695 | 0.170413 | 0.281977 | Diet |
| 2-arachidonoylglycerol (20:4) | 0.333827 | 0.01343 | 0.0268 | Diet |
| docosahexaenoate (DHA; | 0.099721 | 0.022662 | 0.204594 | Diet |
| 22:6n3) | | | | |
| alpha-hydroxyisocaproate | 0.190883 | 0.068172 | 0.288966 | Sex |
| phenyllactate (PLA) | 0.107697 | 0.018901 | 0.156605 | Sex |
| N-acetylaspartate (NAA) | 0.569841 | 0.052896 | 0.03993 | Anthropometrics |
| dehydroisoandrosterone | 0.21087 | 0.059998 | 0.224529 | Age |
| sulfate (DHEA-S) + | | | | |
| androstenediol | | | | |
| (3beta,17beta) monosulfate | | | | |
| (1) | | | | |
| acetylcarnitine (C2) | 0.242397 | 0.032595 | 0.101875 | Diet |
| 1-palmitoylglycerol (16:0) | 0.200892 | 0.02802 | 0.111459 | Cardiometabolic |
| tartronate (hydroxymalonate) | 0.367069 | 0.046048 | 0.079399 | Diet |
| oxalate (ethanedioate) | 0.299409 | 0.085133 | 0.199205 | Diet |
| 2,3-dihydroxypyridine | 0.332366 | 0.167731 | 0.336927 | Diet |
| 1-oleoylglycerol (18:1) | 0.075271 | 0.009936 | 0.122073 | Cardiometabolic |
| 2-oleoylglycerol (18:1) | 0.237011 | 0.020489 | 0.065958 | Anthropometrics |
| 2-linoleoylglycerol (18:2) | 0.42404 | 0.020463 | 0.027794 | Sex |
| N-acetylglycine | 0.363161 | 0.044588 | 0.078189 | Diet |
| threonate | 0.436455 | 0.101397 | 0.130922 | Diet |
| indoleacetate | 0.617335 | 0.053492 | 0.033158 | Diet |
| 1-methylhistidine | 0.308979 | 0.080211 | 0.179388 | Sex |
| isobutyrylcarnitine (C4) | 0.392293 | 0.0513 | 0.079469 | Diet |
| glycolithocholate | 0.766774 | 0.092844 | 0.02824 | Time of day |
| indolepropionate | 0.705066 | 0.155376 | 0.064995 | Diet |
| trigonelline (N′- | 0.26634 | 0.13141 | 0.361983 | Diet |
| methylnicotinate) | | | | |
| dodecanedioate | 0.395794 | 0.054011 | 0.082451 | Diet |
| 3-methylxanthine | 0.266129 | 0.046021 | 0.126905 | Diet |
| gamma-glutamylvaline | 0.268344 | 0.088646 | 0.2417 | Diet |
| 5-hydroxyhexanoate | 0.888408 | 0.127023 | 0.015955 | Time of day |
| propionylglycine | 0.293976 | 0.032648 | 0.078409 | Diet |
| propionylcarnitine (C3) | 0.397752 | 0.076808 | 0.116297 | Anthropometrics |
| 3-hydroxy-2-ethylpropionate | 0.358802 | 0.05234 | 0.093534 | Diet |
| 3-carboxy-4-methyl-5-propyl- | 0.079797 | 0.04595 | 0.529887 | Diet |
| 2-furanpropanoate (CMPF) | | | | |
| I-urobilinogen | 0.667928 | 0.086 | 0.042756 | Time of day |
| tauro-beta-muricholate | 0.334622 | 0.042089 | 0.083692 | Anthropometrics |
| N-acetylarginine | 0.171476 | 0.021006 | 0.101494 | Cardiometabolic |
| piperine | 0.379707 | 0.025898 | 0.042307 | Seasonal effects |
| myristoylcarnitine | 0.154516 | 0.021401 | 0.117106 | Diet |
| (C14) + palmitoylcarnitine | | | | |
| (C16) | | | | |
| epiandrosterone | 0.224373 | 0.03567 | 0.123306 | Sex |
| sulfate + androsterone sulfate | | | | |
| alpha-hydroxyisovalerate | 0.266863 | 0.07422 | 0.2039 | Sex |
| p-cresol sulfate | 0.940319 | 0.366329 | 0.023251 | Diet |
| DSGEGDFXAEGGGVR* + | 0.233223 | 0.02929 | 0.096297 | Diet |
| Fibrinopeptide A (5- | | | | |
| 16)* + Fibrinopeptide A (7- | | | | |
| 16)* + Fibrinopeptide A (3- | | | | |
| 16)** | | | | |
| stearoylcarnitine (C18) | 0.126388 | 0.02328 | 0.160912 | Diet |
| isovalerylcarnitine (C5) | 0.263186 | 0.057778 | 0.161754 | Cardiometabolic |
| 1,7- | 0.369806 | 0.266644 | 0.454393 | Diet |
| dimethylurate + theophylline | | | | |
| 1-methylurate + 1,3- | 0.379695 | 0.304028 | 0.496689 | Diet |
| dimethylurate | | | | |
| 5-acetylamino-6- | 0.366103 | 0.137479 | 0.238041 | Diet |
| formylamino-3-methyluracil | | | | |
| 5-acetylamino-6-amino-3- | 0.361042 | 0.269664 | 0.477241 | Diet |
| methyluracil | | | | |
| 1-methylxanthine | 0.354964 | 0.288703 | 0.524628 | Diet |
| N1-methylinosine | 0.269149 | 0.034367 | 0.093322 | Diet |
| 4-hydroxyhippurate | 0.357954 | 0.019849 | 0.035602 | Diet |
| 7-methylguanine | 0.563232 | 0.092589 | 0.0718 | Diet |
| 3-methylcytidine | 0.550664 | 0.048046 | 0.039205 | Age |
| N1-Methyl-2-pyridone-5- | 0.217095 | 0.043443 | 0.156667 | Diet |
| carboxamide | | | | |
| 1-docosahexaenoylglycerol | 0.255119 | 0.059142 | 0.172678 | Diet |
| (22:6) | | | | |
| gamma-glutamylisoleucine* | 0.206551 | 0.053026 | 0.203694 | Anthropometrics |
| oleoylcarnitine (C18:1) | 0.192587 | 0.012451 | 0.052201 | Sex |
| gamma-glutamyl-2- | 0.484066 | 0.052892 | 0.056374 | Diet |
| aminobutyrate | | | | |
| 2-methylbutyrylcarnitine (C5) | 0.383153 | 0.093097 | 0.149879 | Sex |
| phenol sulfate | 0.92377 | 0.131786 | 0.010875 | Age |
| pyroglutamine* | 0.101858 | 0.040868 | 0.360359 | Sex |
| 2-hydroxy-3-methylvalerate | 0.1959 | 0.053295 | 0.218756 | Sex |
| dimethyl sulfoxide (DMSO) | 0.409281 | 0.081046 | 0.116975 | Diet |
| glutarylcarnitine (C5-DC) | 0.071028 | 0.015927 | 0.208308 | Sex |
| tiglylcarnitine (C5:1-DC) | 0.179638 | 0.040058 | 0.182934 | Diet |
| catechol sulfate + O- | 0.31156 | 0.0758 | 0.167492 | Diet |
| methylcatechol sulfate | | | | |
| 7-alpha-hydroxy-3-oxo-4- | 0.422661 | 0.033347 | 0.04555 | Sex |
| cholestenoate (7-Hoca) | | | | |
| tetradecanedioate | 0.293974 | 0.016216 | 0.038946 | Sex |
| 1-myristoylglycerol (14:0) | 0.182225 | 0.029043 | 0.130336 | Anthropometrics |
| 3-(3- | 0.453199 | 0.087249 | 0.10527 | Diet |
| hydroxyphenyl)propionate + 3- | | | | |
| hydroxyhippurate + X - 12543 | | | | |
| ectoine | 0.207595 | 0.02318 | 0.088479 | Sex |
| glycolithocholate sulfate* | 0.814685 | 0.206354 | 0.046939 | Sex |
| taurolithocholate 3-sulfate | 0.855405 | 0.190948 | 0.032277 | Anthropometrics |
| deoxycarnitine | 0.146301 | 0.054833 | 0.319963 | Sex |
| 1-ribosyl-imidazoleacetate* | 0.357369 | 0.028333 | 0.050949 | Diet |
| indoleacetylglutamine | 0.281227 | 0.013213 | 0.03377 | Age |
| hexanoylglutamine | 0.205895 | 0.030468 | 0.117512 | Macronutrients |
| tryptophan betaine | 0.223764 | 0.081053 | 0.281174 | Diet |
| 4-ethylphenylsulfate | 0.276416 | 0.076357 | 0.199882 | Diet |
| 3-methyladipate | 0.373318 | 0.031094 | 0.052197 | Diet |
| o-cresol sulfate | 0.058764 | 0.019915 | 0.318978 | Time of day |
| 4-allylphenol sulfate | 0.461766 | 0.059053 | 0.068832 | Diet |
| N-methylproline + stachydrine | 0.168014 | 0.043504 | 0.215429 | Diet |
| beta-cryptoxanthin | 0.260259 | 0.111969 | 0.318252 | Diet |
| 5alpha-androstan- | 0.125245 | 0.04892 | 0.341679 | Sex |
| 3beta,17beta-diol disulfate | | | | |
| 5alpha-pregnan- | 0.214237 | 0.029969 | 0.109918 | Age |
| 3beta,20alpha-diol disulfate | | | | |
| glycocholenate sulfate* | 0.16895 | 0.013276 | 0.065304 | Diet |
| androstenediol (3beta,17beta) | 0.167763 | 0.030466 | 0.151134 | Sex |
| disulfate (1) | | | | |
| pregnen-diol disulfate | 0.30319 | 0.054851 | 0.126061 | Sex |
| C21H34O8S2* + pregnenetriol | | | | |
| disulfate* | | | | |
| androstenediol (3beta,17beta) | 0.225886 | 0.05468 | 0.187388 | Sex |
| disulfate (2) | | | | |
| 21-hydroxypregnenolone | 0.199878 | 0.022581 | 0.090394 | Age |
| disulfate | | | | |
| 5alpha-androstan- | 0.254411 | 0.100841 | 0.295528 | Sex |
| 3alpha,17alpha-diol | | | | |
| monosulfate | | | | |
| 5alpha-pregnan- | 0.237568 | 0.047354 | 0.151974 | Age |
| 3beta,20alpha-diol | | | | |
| monosulfate (2) + 5alpha- | | | | |
| pregnan-3beta,20beta-diol | | | | |
| monosulfate (1) | | | | |
| 5alpha-pregnan-3(alpha or | 0.216205 | 0.029103 | 0.105504 | Age |
| beta),20beta-diol disulfate | | | | |
| 5alpha-androstan- | 0.123633 | 0.096467 | 0.683802 | Sex |
| 3alpha,17beta-diol disulfate | | | | |
| 5alpha-androstan- | 0.147812 | 0.030066 | 0.173341 | Sex |
| 3alpha,17beta-diol | | | | |
| monosulfate (1) + 5alpha- | | | | |
| androstan-3beta,17beta-diol | | | | |
| monosulfate (2) | | | | |
| 5alpha-androstan- | 0.557296 | 0.213581 | 0.169664 | Sex |
| 3beta,17alpha-diol disulfate | | | | |
| androstenediol (3alpha, | 0.187007 | 0.051007 | 0.221749 | Sex |
| 17alpha) monosulfate (2) | | | | |
| androstenediol (3alpha, | 0.16319 | 0.049941 | 0.25609 | Sex |
| 17alpha) monosulfate (3) | | | | |
| 5alpha-pregnan-3beta-ol,20-one | 0.206087 | 0.027644 | 0.106492 | Age |
| sulfate | | | | |
| 4-hydroxycoumarin | 0.80298 | 0.10379 | 0.025466 | Time of day |
| pregn steroid monosulfate | 0.238328 | 0.076802 | 0.245452 | Age |
| C21H34O5S* + pregnenolone | | | | |
| sulfate | | | | |
| sphingomyelin (d18:1/18:1, | 0.335254 | 0.045242 | 0.089706 | Sex |
| d18:2/18:0) | | | | |
| 17alpha- | 0.174737 | 0.039231 | 0.185283 | Age |
| hydroxypregnenolone 3- | | | | |
| sulfate | | | | |
| andro steroid monosulfate | 0.233965 | 0.024136 | 0.079025 | Age |
| C19H28O6S (1)* + 16a- | | | | |
| hydroxy DHEA 3-sulfate | | | | |
| ergothioneine | 0.419401 | 0.105559 | 0.146132 | Diet |
| S-methylmethionine | 0.273073 | 0.04592 | 0.122239 | Diet |
| indole-3-carboxylic acid | 0.701777 | 0.044805 | 0.01904 | Time of day |
| tridecenedioate (C13:1-DC)* | 0.136806 | 0.029936 | 0.188882 | Diet |
| N-acetyl-3-methylhistidine* | 0.245217 | 0.03119 | 0.096003 | Diet |
| 7-methylurate | 0.307947 | 0.049891 | 0.11212 | Diet |
| N-acetyl-cadaverine | 0.780853 | 0.184978 | 0.051914 | Diet |
| cinnamoylglycine | 0.742039 | 0.246357 | 0.085643 | Diet |
| 2,3-dihydroxyisovalerate | 0.468748 | 0.030495 | 0.034561 | Sex |
| cysteinylglycine disulfide* | 0.07097 | 0.014108 | 0.184685 | Anthropometrics |
| isoursodeoxycholate | 0.926202 | 0.201975 | 0.016093 | Anthropometrics |
| formiminoglutamate | 0.307344 | 0.056343 | 0.126979 | Cardiometabolic |
| L-urobilin | 0.85654 | 0.151148 | 0.025315 | Time of day |
| S-methylcysteine + S- | 0.148468 | 0.040269 | 0.230963 | Diet |
| methylcysteine sulfoxide | | | | |
| androsterone glucuronide | 0.050174 | 0.012288 | 0.232627 | Sex |
| argininate* | 0.270733 | 0.0546 | 0.147073 | Diet |
| 1-lignoceroyl-GPC (24:0) | 0.385846 | 0.091729 | 0.146006 | Diet |
| 1-(1-enyl-palmitoyl)-GPC | 0.246711 | 0.015476 | 0.047253 | Anthropometrics |
| (P-16:0)* | | | | |
| 1-methyl-5- | 0.253327 | 0.125742 | 0.37062 | Diet |
| imidazoleacetate + X - 13835 | | | | |
| glycoursodeoxycholate | 0.926769 | 0.146924 | 0.01161 | Diet |
| tauroursodeoxycholate | 0.643116 | 0.044636 | 0.02477 | Time of day |
| 15-methylpalmitate + myristate | 0.071405 | 0.014394 | 0.187193 | Diet |
| (14:0) | | | | |
| 1-(1-enyl-palmitoyl)-GPE (P- | 0.2704 | 0.040271 | 0.108661 | Diet |
| 16:0)* + 1-(1-enyl-oleoyl)- | | | | |
| GPE(P-18:1)* | | | | |
| 1-(1-enyl-stearoyl)-GPE | 0.223621 | 0.049078 | 0.170391 | Diet |
| (P-18:0)* | | | | |
| N-oleoyltaurine | 0.308874 | 0.023147 | 0.051792 | Diet |
| linoleoylcarnitine (C18:2)* | 0.166332 | 0.028811 | 0.144404 | Sex |
| leucylalanine | 0.074872 | 0.012745 | 0.15748 | Diet |
| N-palmitoyltaurine | 0.357112 | 0.021222 | 0.038204 | Anthropometrics |
| trimethylamine N-oxide | 0.351416 | 0.041943 | 0.077412 | Diet |
| imidazole propionate | 0.50283 | 0.058109 | 0.057455 | Sex |
| pregnanediol-3-glucuronide | 0.343259 | 0.045552 | 0.087153 | Age |
| 3-hydroxybutyrylcarnitine (1) | 0.297005 | 0.064329 | 0.152263 | Macronutrients |
| 5-hydroxymethyl-2-furoic | 0.437487 | 0.036237 | 0.046592 | Diet |
| acid | | | | |
| N-acetylcarnosine | 0.189673 | 0.120697 | 0.515647 | Sex |
| margaroylcarnitine* | 0.442252 | 0.037078 | 0.046761 | Diet |
| N-methyltaurine | 0.322565 | 0.047903 | 0.100604 | Diet |
| glycohyocholate + X - 22716 | 0.353491 | 0.055731 | 0.101928 | Diet |
| 4-methylcatechol sulfate | 0.84032 | 0.144559 | 0.02747 | Time of day |
| 3-methyl catechol sulfate | 0.390624 | 0.185658 | 0.289628 | Diet |
| (1) + 3-methyl catechol sulfate | | | | |
| (2) | | | | |
| indolin-2-one | 0.874083 | 0.143459 | 0.020666 | Sex |
| 3-acetylphenol sulfate | 0.428795 | 0.101779 | 0.135582 | Diet |
| sphingomyelin (d18:1/14:0, | 0.30794 | 0.112004 | 0.251715 | Diet |
| d16:1/16:0)* | | | | |
| N-delta-acetylornithine | 0.333084 | 0.100466 | 0.201158 | Diet |
| acisoga | 0.414816 | 0.043529 | 0.061406 | Diet |
| benzoylcarnitine* | 0.617258 | 0.099975 | 0.061992 | Diet |
| N-formylanthranilic acid | 0.288919 | 0.012053 | 0.029664 | Diet |
| N2,N5-diacetylornithine | 0.359311 | 0.10592 | 0.188867 | Diet |
| 1H-indole-7-acetic acid | 0.570809 | 0.086686 | 0.065179 | Time of day |
| 3-(3- | 0.312026 | 0.042273 | 0.093206 | Diet |
| hydroxyphenyl)propionate | | | | |
| sulfate | | | | |
| methyl glucopyranoside | 0.357751 | 0.131982 | 0.236939 | Diet |
| (alpha + beta) | | | | |
| sphingomyelin (d18:2/14:0, | 0.305079 | 0.091381 | 0.208152 | Sex |
| d18:1/14:1)* | | | | |
| 5alpha-androstan- | 0.154317 | 0.142246 | 0.779535 | Sex |
| 3alpha,17beta-diol | | | | |
| monosulfate (2) | | | | |
| 4-hydroxychlorothalonil | 0.365537 | 0.041478 | 0.071994 | Diet |
| 3-hydroxypyridine sulfate + | 0.382638 | 0.224392 | 0.362042 | Diet |
| X - 23655 | | | | |
| phenylacetylcarnitine | 0.883782 | 0.18954 | 0.024925 | Sex |
| arabonate/xylonate | 0.358472 | 0.066979 | 0.119867 | Diet |
| pregnanolone/allopregnanolone | 0.147609 | 0.02298 | 0.132703 | Age |
| sulfate | | | | |
| p-cresol-glucuronide* | 0.928391 | 0.401115 | 0.030939 | Anthropometrics |
| 6-hydroxyindole sulfate + | 0.789913 | 0.123466 | 0.032837 | Diet |
| X - 21310 | | | | |
| sphingomyelin (d18:1/22:1, | 0.337653 | 0.052896 | 0.103761 | Sex |
| d18:2/22:0, d16:1/24:1)* | | | | |
| sphingomyelin (d17:1/16:0, | 0.255659 | 0.104608 | 0.304562 | Diet |
| d18:1/15:0, | | | | |
| d16:1/17:0)* + sphingomyelin | | | | |
| (d18:1/17:0, d17:1/18:0, | | | | |
| d19:1/16:0) | | | | |
| 3-methoxycatechol sulfate | 0.409823 | 0.029801 | 0.042916 | Diet |
| (1) + 1,2,3-benzenetriol | | | | |
| sulfate (2) | | | | |
| arabitol/xylitol | 0.439269 | 0.051734 | 0.066039 | Age |
| citraconate/glutaconate + maleate | 0.425319 | 0.167781 | 0.226702 | Diet |
| adipoylcarnitine (C6-DC) | 0.226355 | 0.027684 | 0.094619 | Diet |
| glycodeoxycholate sulfate | 0.377098 | 0.02516 | 0.04156 | Seasonal effects |
| taurodeoxycholic acid 3- | 0.482548 | 0.051577 | 0.055307 | Time of day |
| sulfate | | | | |
| phenol glucuronide | 0.779761 | 0.054834 | 0.015488 | Sex |
| linoleoyl ethanolamide | 0.282102 | 0.009612 | 0.024462 | Time of day |
| 1-stearoyl-2- | 0.130097 | 0.035116 | 0.234806 | Diet |
| docosahexaenoyl-GPC | | | | |
| (18:0/22:6) | | | | |
| 2-hydroxybutyrate/2- | 0.179884 | 0.041659 | 0.189928 | Diet |
| hydroxyisobutyrate | | | | |
| 2-hydroxylaurate | 0.208051 | 0.05741 | 0.218534 | Sex |
| sphingomyelin (d18:2/24:1, | 0.158306 | 0.029344 | 0.156017 | Sex |
| d18:1/24:2)* | | | | |
| 1-palmitoyl-2-palmitoleoyl- | 0.271175 | 0.047007 | 0.126339 | Diet |
| GPC (16:0/16:1)* + 1- | | | | |
| myristoyl-2-palmitoyl-GPC | | | | |
| (14:0/16:0) | | | | |
| gamma-tocopherol/beta- | 0.295388 | 0.041578 | 0.09918 | Diet |
| tocopherol | | | | |
| 1-(1-enyl-stearoyl)-2- | 0.187762 | 0.073886 | 0.319622 | Diet |
| arachidonoyl-GPE (P- | | | | |
| 18:0/20:4)* | | | | |
| 1-(1-enyl-palmitoyl)-2- | 0.286155 | 0.137336 | 0.342599 | Diet |
| arachidonoyl-GPE (P- | | | | |
| 16:0/20:4)* | | | | |
| 1-(1-enyl-palmitoyl)-2-oleoyl- | 0.221341 | 0.057445 | 0.202087 | Anthropometrics |
| GPC (P-16:0/18:1)* | | | | |
| 1-(1-enyl-palmitoyl)-2- | 0.175395 | 0.047465 | 0.223155 | Diet |
| arachidonoyl-GPC (P- | | | | |
| 16:0/20:4)* | | | | |
| sphingomyelin (d18:0/18:0, | 0.205885 | 0.022245 | 0.085801 | Cardiometabolic |
| d19:0/17:0)* + sphingomyelin | | | | |
| (d18:0/20:0, d16:0/22:0)* | | | | |
| myristoyl | 0.241208 | 0.049654 | 0.156202 | Diet |
| dihydrosphingomyelin | | | | |
| (d18:0/14:0)* | | | | |
| 1-(1-enyl-palmitoyl)-2- | 0.32451 | 0.055708 | 0.115959 | Diet |
| linoleoyl-GPE (P-16:0/18:2)* | | | | |
| 1-oleoyl-2-docosahexaenoyl- | 0.231722 | 0.033848 | 0.112222 | Diet |
| GPC (18:1/22:6)* | | | | |
| 1-palmitoyl-2-gamma- | 0.242427 | 0.01428 | 0.044623 | Anthropometrics |
| linolenoyl-GPC | | | | |
| (16:0/18:3n6)* | | | | |
| 1-(1-enyl-palmitoyl)-2- | 0.211229 | 0.016329 | 0.060975 | Sex |
| palmitoleoyl-GPC (P- | | | | |
| 16:0/16:1)* | | | | |
| 1-oleoyl-2-docosahexaenoyl- | 0.281112 | 0.024935 | 0.063767 | Diet |
| GPE (18:1/22:6)* | | | | |
| 2-methylserine | 0.253501 | 0.028295 | 0.083321 | Diet |
| glycocholate glucuronide (1) | 0.837853 | 0.071558 | 0.013848 | Drugs |
| 14-HDoHE/17-HDoHE | 0.444463 | 0.032585 | 0.040728 | Seasonal effects |
| catechol glucuronide | 0.238255 | 0.026107 | 0.083469 | Diet |
| palmitoloelycholine | 0.482857 | 0.026824 | 0.028729 | Sex |
| eicosapentaenoylcholine | 0.37232 | 0.018401 | 0.031021 | Cardiometabolic |
| caffeic acid sulfate | 0.24934 | 0.050395 | 0.151718 | Diet |
| 2,3-dihydroxy-2- | 0.28063 | 0.037595 | 0.096372 | Diet |
| methylbutyrate | | | | |
| linoleoyl-arachidonoyl- | 0.15431 | 0.011884 | 0.06513 | Cardiometabolic |
| glycerol (18:2/20:4) | | | | |
| [2]* + linoleoyl-arachidonoyl- | | | | |
| glycerol (18:2/20:4) [1]* | | | | |
| perfluorooctanesulfonic acid | 0.168419 | 0.051079 | 0.252208 | Diet |
| (PFOS) | | | | |
| 2-hydroxynervonate* | 0.354288 | 0.03507 | 0.063918 | Diet |
| N-palmitoyl- | 0.577092 | 0.042882 | 0.031425 | Diet |
| heptadecasphingosine | | | | |
| (d17:1/16:0)* | | | | |
| ceramide (d18:1/14:0, | 0.335338 | 0.070115 | 0.138972 | Diet |
| d16:1/16:0)* | | | | |
| glycosyl ceramide | 0.319625 | 0.02929 | 0.062349 | Sex |
| (d18:2/24:1, d18:1/24:2)* | | | | |
| sphingomyelin (d18:1/19:0, | 0.281767 | 0.08422 | 0.21468 | Diet |
| d19:1/18:0)* + sphingomyelin | | | | |
| (d18:1/21:0, d17:1/22:0, | | | | |
| d16:1/23:0)* | | | | |
| sphingomyelin (d18:2/21:0, | 0.323837 | 0.086469 | 0.180544 | Sex |
| d16:2/23:0)* + sphingomyelin | | | | |
| (d18:2/23:0, d18:1/23:1, | | | | |
| d17:1/24:1)* | | | | |
| sphingomyelin (d18:2/23:1)* | 0.257535 | 0.083923 | 0.241946 | Diet |
| sphingomyelin (d17:2/16:0, | 0.342481 | 0.109936 | 0.211062 | Diet |
| d18:2/15:0)* | | | | |
| linolenoylcarnitine (C18:3)* | 0.210918 | 0.020812 | 0.07786 | Sex |
| cerotoylcarnitine (C26)* | 0.11618 | 0.013297 | 0.101154 | Sex |
| ximenoylcarnitine (C26:1)* | 0.265347 | 0.020312 | 0.056238 | Age |
| arachidonoylcarnitine (C20:4) | 0.094692 | 0.018103 | 0.173076 | Sex |
| docosahexaenoylcarnitine | 0.317392 | 0.017468 | 0.037569 | Diet |
| (C22:6)* | | | | |
| N-trimethyl 5-aminovalerate | 0.165255 | 0.029086 | 0.14692 | Diet |
| carotene diol (2) + carotene | 0.460312 | 0.11677 | 0.136906 | Diet |
| diol (1) | | | | |
| carotene diol (3) | 0.141962 | 0.013149 | 0.079477 | Diet |
| hydroxy-CMPF* | 0.039891 | 0.021204 | 0.51034 | Diet |
| dodecenedioate (C12:1-DC)* | 0.192548 | 0.033906 | 0.142187 | Time of day |
| 3-carboxy-4-methyl-5-pentyl- | 0.334037 | 0.070428 | 0.14041 | Diet |
| 2-furanpropionate (3- | | | | |
| CMPFP)** | | | | |
| glucuronide of C10H18O2 | 0.343845 | 0.017662 | 0.033704 | Seasonal effects |
| (7)* | | | | |
| perfluorooctanoate (PFOA) | 0.376413 | 0.027342 | 0.045295 | Anthropometrics |
| N-methylhydroxyproline** | 0.217206 | 0.022951 | 0.082713 | Diet |
| N,N,N-trimethyl- | 0.096878 | 0.043365 | 0.404257 | Sex |
| alanylproline betaine (TMAP) | | | | |
| gamma-glutamylcitrulline* | 0.484649 | 0.052195 | 0.055501 | Sex |
| glycine conjugate of | 0.302041 | 0.061761 | 0.142718 | Diet |
| C10H14O2 (1)* | | | | |
| N-acetyl-isoputreanine* | 0.324644 | 0.030469 | 0.063385 | Diet |
| 2-naphthol sulfate | 0.477464 | 0.037791 | 0.041359 | LifeStyle |
| dihydrocaffeate sulfate (2) | 0.303748 | 0.046147 | 0.105779 | Diet |
| 2,6-dihydroxybenzoic acid | 0.166009 | 0.036423 | 0.182979 | Diet |
| 2,3-dihydroxy-5-methylthio- | 0.096753 | 0.02623 | 0.24487 | Cardiometabolic |
| 4-pentenoate (DMTPA)* | | | | |
| taurochenodeoxycholic acid | 0.357058 | 0.024331 | 0.043812 | Time of day |
| 3-sulfate | | | | |
| eicosenedioate (C20:1-DC)* | 0.232762 | 0.06327 | 0.208553 | Diet |
| hydroxy-N6,N6,N6- | 0.197005 | 0.040766 | 0.166161 | Sex |
| trimethyllysine* | | | | |
| picolinoylglycine | 0.337869 | 0.051426 | 0.100781 | Age |
| sarcosine | 0.33884 | 0.023562 | 0.045975 | Time of day |
| glycerate | 0.691439 | 0.046054 | 0.020552 | Sex |
| N-acetylmethionine | 0.470656 | 0.042956 | 0.048313 | Seasonal effects |
| thyroxine | 0.299497 | 0.027143 | 0.063486 | Sex |
| alpha-tocopherol | 0.430181 | 0.052832 | 0.069982 | Age |
| vanillylmandelate (VMA) | 0.112075 | 0.02733 | 0.216523 | Age |
| chenodeoxycholate | 0.652154 | 0.027443 | 0.014637 | Time of day |
| 2-aminobutyrate | 0.267642 | 0.074972 | 0.205149 | Diet |
| urate | 0.146965 | 0.062233 | 0.361222 | Anthropometrics |
| ursodeoxycholate | 0.817802 | 0.152604 | 0.033999 | Sex |
| 4-hydroxyphenylpyruvate | 0.142653 | 0.012647 | 0.076009 | Diet |
| isocitrate | 0.303912 | 0.034287 | 0.078531 | Diet |
| creatine | 0.088312 | 0.023386 | 0.241425 | Diet |
| cys-gly, oxidized | 0.109975 | 0.013287 | 0.107532 | Sex |
| choline | 0.237484 | 0.013308 | 0.042728 | Diet |
| anthranilate | 0.78757 | 0.114992 | 0.031017 | Age |
| cholate | 0.609831 | 0.086449 | 0.05531 | Time of day |
| N-palmitoyl-sphingosine | 0.52251 | 0.059534 | 0.054405 | Age |
| (d18:1/16:0) | | | | |
| stearoyl sphingomyelin | 0.345845 | 0.064702 | 0.122383 | Diet |
| (d18:1/18:0) | | | | |
| N-stearoyl-sphingosine | 0.30437 | 0.049542 | 0.113227 | Diet |
| (d18:1/18:0)* | | | | |
| taurodeoxycholate | 0.958573 | 0.095894 | 0.004144 | Macronutrients |
| N6,N6,N6-trimethyllysine | 0.227981 | 0.038408 | 0.130061 | Sex |
| 3-(4-hydroxyphenyl)lactate | 0.184398 | 0.062185 | 0.27505 | Sex |
| biliverdin + bilirubin (E,E)* | 0.226841 | 0.032125 | 0.109494 | Sex |
| 3-hydroxybutyrate (BHBA) | 0.193033 | 0.037267 | 0.155792 | Macronutrients |
| creatinine | 0.145844 | 0.083478 | 0.4889 | Sex |
| cystine | 0.193485 | 0.038517 | 0.160552 | Age |
| deoxycholate | 0.706467 | 0.035508 | 0.014753 | Age |
| gamma-glutamylglutamate | 0.271228 | 0.048136 | 0.129338 | Anthropometrics |
| glutarate (pentanedioate) | 0.621059 | 0.087665 | 0.053489 | Sex |
| guanidinoacetate | 0.13674 | 0.021507 | 0.135779 | Sex |
| myo-inositol | 0.511408 | 0.070411 | 0.06727 | Time of day |
| isoleucine | 0.189793 | 0.031782 | 0.135676 | Diet |
| 2-aminoadipate | 0.224225 | 0.057367 | 0.198478 | Diet |
| citrulline | 0.446846 | 0.046122 | 0.057095 | Age |
| leucine + gamma- | 0.288715 | 0.06693 | 0.164891 | Anthropometrics |
| glutamylleucine | | | | |
| malate | 0.321709 | 0.033531 | 0.070697 | Diet |
| nicotinamide | 0.127478 | 0.018211 | 0.124646 | Time of day |
| ornithine | 0.167702 | 0.018443 | 0.091533 | Anthropometrics |
| phytanate | 0.180388 | 0.033068 | 0.150248 | Diet |
| proline | 0.289837 | 0.025212 | 0.061775 | Sex |
| retinol (Vitamin A) | 0.250333 | 0.027347 | 0.081897 | Cardiometabolic |
| taurine | 0.599726 | 0.035708 | 0.023833 | Seasonal effects |
| urea | 0.260789 | 0.051152 | 0.144991 | Diet |
| glutamate | 0.18149 | 0.040377 | 0.182097 | Anthropometrics |
| valine | 0.186757 | 0.032666 | 0.142244 | Diet |
| caffeine + 1,3,7-trimethylurate | 0.394336 | 0.270008 | 0.414708 | Diet |
| caprate (10:0) | 0.1687 | 0.02295 | 0.11309 | Diet |
| alpha-ketoglutarate | 0.226222 | 0.035558 | 0.121624 | Anthropometrics |
| X - 01911 | 0.223063 | 0.016043 | 0.055877 | Sex |
| X - 11261 | 0.315102 | 0.048919 | 0.10633 | Diet |
| X - 11299 + X - 11483 | 0.754082 | 0.025772 | 0.008405 | Macronutrients |
| X - 11308 | 0.25473 | 0.119144 | 0.348583 | Diet |
| X - 11315 | 0.266674 | 0.135361 | 0.372229 | Diet |
| X - 11378 | 0.158686 | 0.052512 | 0.278406 | Sex |
| X - 11381 | 0.205243 | 0.072687 | 0.281463 | Diet |
| X - 11444 | 0.083443 | 0.018535 | 0.203599 | Anthropometrics |
| X - 11470 | 0.068143 | 0.011831 | 0.16179 | Time of day |
| X - 11478 | 0.42134 | 0.056492 | 0.077585 | Diet |
| X - 11485 | 0.232614 | 0.023795 | 0.078497 | Diet |
| X - 11491 | 0.205863 | 0.018237 | 0.070353 | Anthropometrics |
| X - 11640 | 0.403971 | 0.053868 | 0.079478 | Diet |
| X - 11795 | 0.110742 | 0.034854 | 0.279879 | Diet |
| X - 11843 + X - 11850 + | 0.953159 | 0.479096 | 0.023544 | Macronutrients |
| X - 12013 | | | | |
| X - 11849 | 0.102283 | 0.019434 | 0.170569 | Diet |
| X - 11852 | 0.436372 | 0.014419 | 0.018625 | Diet |
| X - 11858 | 0.051082 | 0.017322 | 0.321775 | Diet |
| X - 11880 + X - 11372 | 0.278849 | 0.137921 | 0.356687 | Diet |
| X - 12063 | 0.123077 | 0.041351 | 0.294627 | Anthropometrics |
| X - 12101 | 0.178396 | 0.028461 | 0.131076 | Diet |
| X - 12126 | 0.846036 | 0.251003 | 0.045678 | Diet |
| X - 12206 | 0.47295 | 0.034313 | 0.038238 | Diet |
| X - 12212 | 0.503736 | 0.057528 | 0.056675 | Diet |
| X - 12216 | 0.933304 | 0.234275 | 0.016742 | Time of day |
| X - 12221 | 0.316174 | 0.048395 | 0.10467 | Diet |
| 4-ethylcatechol sulfate | 0.383336 | 0.182636 | 0.293803 | Diet |
| X - 12261 | 0.964726 | 0.403181 | 0.014742 | Cardiometabolic |
| X - 12283 | 0.640942 | 0.128873 | 0.072195 | Diet |
| X - 12306 | 0.592862 | 0.17703 | 0.121572 | Diet |
| X - 12329 + N- | 0.401011 | 0.113761 | 0.169925 | Diet |
| (2-furoyl)glycine | | | | |
| X - 12411 | 0.256335 | 0.036709 | 0.106498 | Time of day |
| X - 12544 | 0.283296 | 0.043788 | 0.110778 | Diet |
| X - 12718 | 0.619573 | 0.14593 | 0.089603 | Age |
| X - 12730 | 0.424132 | 0.107163 | 0.1455 | Diet |
| X - 12738 | 0.429681 | 0.094466 | 0.125386 | Diet |
| X - 12798 | 0.278326 | 0.024121 | 0.062544 | Diet |
| X - 12816 | 0.527171 | 0.311965 | 0.279807 | Diet |
| X - 12822 | 0.569941 | 0.081195 | 0.061267 | Sex |
| X - 12830 | 0.568434 | 0.037847 | 0.028734 | Time of day |
| X - 12837 | 0.590399 | 0.222416 | 0.154305 | Diet |
| X - 12851 | 0.938032 | 0.269466 | 0.017802 | Sex |
| X - 12906 | 0.182565 | 0.03077 | 0.137774 | Time of day |
| X - 13431 | 0.297652 | 0.042781 | 0.100947 | Diet |
| X - 13684 | 0.144862 | 0.019582 | 0.115595 | Sex |
| X - 13703 + X - 13255 | 0.387965 | 0.060009 | 0.094668 | Diet |
| X - 13729 | 0.759983 | 0.18146 | 0.057308 | Diet |
| X - 13844 | 0.110693 | 0.038524 | 0.309501 | Diet |
| X - 13866 | 0.154912 | 0.025277 | 0.137895 | Diet |
| X - 14082 | 0.484328 | 0.062808 | 0.066872 | Diet |
| X - 14662 | 0.754385 | 0.122946 | 0.040029 | Diet |
| X - 14939 | 0.135131 | 0.036604 | 0.234274 | Diet |
| X - 15461 | 0.442749 | 0.061711 | 0.077671 | Cardiometabolic |
| X - 15503 | 0.131259 | 0.036143 | 0.239213 | Age |
| X - 15728 | 0.582189 | 0.046332 | 0.03325 | Seasonal effects |
| X - 16087 | 0.389475 | 0.080624 | 0.126383 | Diet |
| X - 16124 | 0.945499 | 0.572857 | 0.033021 | Seasonal effects |
| X - 16580 | 0.32773 | 0.051125 | 0.104872 | Diet |
| X - 16654 | 0.879092 | 0.124415 | 0.017112 | Sex |
| X - 16935 | 0.304491 | 0.082094 | 0.187518 | Diet |
| X - 16944 | 0.372807 | 0.03057 | 0.051429 | Diet |
| X - 17145 | 0.390481 | 0.163885 | 0.255816 | Diet |
| X - 17185 | 0.240775 | 0.094821 | 0.298997 | Diet |
| X - 17337 | 0.345807 | 0.044816 | 0.084783 | Diet |
| X - 17354 + X - 22509 | 0.508746 | 0.11158 | 0.107743 | Diet |
| X - 17367 + X - 17325 | 0.207975 | 0.041148 | 0.156702 | Diet |
| X - 17469 | 0.889628 | 0.181012 | 0.022457 | LifeStyle |
| X - 17612 | 0.879879 | 0.200559 | 0.02738 | Time of day |
| X - 17653 | 0.209 | 0.049858 | 0.188697 | Diet |
| X - 17654 | 0.272664 | 0.064005 | 0.170734 | Diet |
| X - 17655 | 0.172795 | 0.024202 | 0.11586 | Diet |
| X - 17676 | 0.221321 | 0.040236 | 0.141564 | Diet |
| X - 18240 | 0.485008 | 0.074912 | 0.079543 | Diet |
| X - 18249 | 0.275114 | 0.115544 | 0.304443 | Diet |
| X - 18606 | 0.340199 | 0.04149 | 0.080469 | Diet |
| X - 18886 | 0.300093 | 0.069255 | 0.161523 | Diet |
| X - 18887 | 0.294542 | 0.019636 | 0.04703 | Time of day |
| X - 18899 | 0.102736 | 0.009298 | 0.081209 | Diet |
| X - 18901 | 0.281107 | 0.048952 | 0.125188 | Diet |
| X - 18914 | 0.244488 | 0.101505 | 0.313669 | Diet |
| X - 18922 | 0.203237 | 0.064215 | 0.251745 | Diet |
| X - 19434 | 0.648943 | 0.075494 | 0.04084 | Time of day |
| X - 21285 | 0.207055 | 0.031312 | 0.119913 | Sex |
| X - 21286 | 0.84225 | 0.121941 | 0.022839 | Time of day |
| X - 21319 | 0.273118 | 0.048592 | 0.129323 | Diet |
| X - 21339 | 0.248849 | 0.101561 | 0.306562 | Diet |
| X - 21364 | 0.163809 | 0.028826 | 0.147146 | Sex |
| X - 21383 | 0.19078 | 0.038465 | 0.163157 | Diet |
| X - 21410 | 0.333429 | 0.042032 | 0.084028 | Diet |
| X - 21442 | 0.315377 | 0.229695 | 0.498624 | Diet |
| X - 21467 | 0.38719 | 0.024764 | 0.039194 | Drugs |
| X - 21657 | 0.412839 | 0.034462 | 0.049014 | Sex |
| X - 21659 + X - 21474 | 0.324629 | 0.035262 | 0.073361 | Seasonal effects |
| X - 21661 + X - 11847 | 0.04478 | 0.010795 | 0.230265 | Diet |
| X - 21736 | 0.392913 | 0.128818 | 0.199036 | Diet |
| X - 21752 | 0.361439 | 0.161664 | 0.285614 | Diet |
| X - 21821 + X - 17351 | 0.697643 | 0.190367 | 0.082505 | Diet |
| X - 21829 | 0.429735 | 0.09914 | 0.131561 | Diet |
| X - 21839 | 0.869517 | 0.046487 | 0.006976 | Time of day |
| X - 21845 | 0.751507 | 0.077756 | 0.025711 | Time of day |
| X - 22162 | 0.637822 | 0.143328 | 0.081386 | Time of day |
| X - 22520 | 0.677537 | 0.097295 | 0.046306 | Time of day |
| X - 22834 | 0.784112 | 0.057478 | 0.015825 | Seasonal effects |
| X - 23314 | 0.343232 | 0.050182 | 0.096022 | Diet |
| X - 23583 | 0.374073 | 0.018852 | 0.031545 | Time of day |
| X - 23585 | 0.412576 | 0.018921 | 0.02694 | Seasonal effects |
| X - 23587 | 0.492778 | 0.073376 | 0.075527 | Diet |
| X - 23639 | 0.374358 | 0.159606 | 0.26674 | Diet |
| X - 23649 | 0.378597 | 0.175167 | 0.287507 | Diet |
| X - 23652 | 0.245643 | 0.132477 | 0.40683 | Diet |
| X - 23654 | 0.211333 | 0.041121 | 0.153456 | Anthropometrics |
| X - 23659 | 0.390389 | 0.071798 | 0.112116 | Diet |
| X - 23680 | 0.225153 | 0.023519 | 0.08094 | Diet |
| X - 23782 | 0.306691 | 0.045979 | 0.10394 | Diet |
| X - 23974 | 0.293538 | 0.025475 | 0.061312 | Diet |
| X - 23997 | 0.939331 | 0.218763 | 0.014129 | LifeStyle |
| X - 24243 | 0.638978 | 0.144722 | 0.081768 | Diet |
| X - 24328 | 0.077111 | 0.0184 | 0.220214 | Sex |
| X - 24337 | 0.232288 | 0.03201 | 0.105794 | Diet |
| X - 24352 | 0.288702 | 0.028808 | 0.070977 | Diet |
| X - 24410 | 0.775694 | 0.192359 | 0.055624 | Diet |
| X - 24435 | 0.354507 | 0.019877 | 0.036192 | Time of day |
| X - 24455 | 0.301566 | 0.013947 | 0.032302 | Time of day |
| X - 24473 | 0.359349 | 0.078371 | 0.139721 | Diet |
| X - 24475 | 0.226309 | 0.064789 | 0.221498 | Diet |
| X - 24512 | 0.120053 | 0.013585 | 0.099577 | Sex |
| X - 24544 | 0.270667 | 0.052693 | 0.141986 | Age |
| X - 24556 | 0.434236 | 0.049206 | 0.06411 | Diet |
| X - 24693 | 0.383104 | 0.064027 | 0.103099 | Diet |
| X - 24736 | 0.408414 | 0.06513 | 0.09434 | Diet |
| X - 24748 | 0.198616 | 0.015027 | 0.060632 | Diet |
| X - 24760 + 3- | 0.280665 | 0.044235 | 0.113372 | Diet |
| hydroxyhippurate sulfate | | | | |
| X - 24801 | 0.145752 | 0.037726 | 0.221109 | Anthropometrics |
| X - 24811 | 0.387112 | 0.286808 | 0.454083 | Diet |
| X - 24947 | 0.459853 | 0.039444 | 0.046332 | Sex |
| X - 24948 | 0.258436 | 0.079687 | 0.228657 | Sex |
| X - 24949 | 0.162358 | 0.059267 | 0.30577 | Diet |
| X - 24951 | 0.323964 | 0.087978 | 0.18359 | Diet |
| X - 24972 | 0.401434 | 0.055144 | 0.082224 | Age |

Identification and Candidate Structures of Microbiome-Related Unknown Compounds

Metabolites that are accurately predicted by the gut microbiome are of particular interest as they may be modulated by perturbing the bacterial community. Since many of the metabolites that were predicted by the gut microbiome with high accuracy are unknown, we sought their identification. Here we provide the chemical identification of 11 compounds and candidate structures for 19 other compounds previously tagged as unknown (Table 9). Among these metabolites are some of those that are predicted by the microbiome with the highest accuracy, including X-11850, X-12261 and X-11843. These were all predicted with R²>0.45 using the microbiome, and are likely to be derivatives of aromatic amino acids, a class of molecules known to be metabolized by the gut microbiome. This list constitutes a major step towards mapping the metabolic producing and modulating potential of the human gut microbiome.

TABLE 9

| Metabolite | | Microbiome |
|---|---|---|
| name | Identified molecule | R² |
| X - 12837 | glucuronide of C19H28O4 (2)* | 0.28 |
| X - 12230 | 4-ethylcatechol sulfate | 0.23 |
| X - 23649 | 3-hydroxypyridine glucuronide | 0.21 |
| X - 12329 | 3-hydroxy-2-methylpyridine sulfate | 0.17 |
| X - 17145 | branched chain 14:0 dicarboxylic acid** | 0.16 |
| X - 14662 | glycoursodeoxycholate sulfate (1) | 0.14 |
| X - 17469 | lithocholic acid sulfate (1) | 0.12 |
| X - 16654 | deoxycholic acid (12 or 24)-sulfate* | 0.12 |
| X - 18249 | 3,5-dichloro-2,6-dihydroxybenzoic acid | 0.09 |
| X - 11640 | enterolactone sulfate | 0.07 |
| X - 18914 | 3-bromo-5-chloro-2,6-dihydroxybenzoic acid* | 0.04 |

| Metabolite | | Microbiome |
|---|---|---|
| name | Candidate structure | R² |
| X - 11850 | aromatic amino acid related metabolite | 0.52 |
| X - 12261 | aromatic amino acid related metabolite | 0.47 |
| X - 11843 | aromatic amino acid related metabolite | 0.46 |
| X - 23655 | pyridine related | 0.31 |
| X - 12126 | aromatic amino acid related metabolite | 0.27 |
| X - 12216 | aromatic amino acid related metabolite | 0.25 |
| X - 24410 | piperidine related | 0.19 |
| X - 17185 | phenol-related | 0.19 |
| X - 12718 | aromatic amino acid related metabolite | 0.17 |
| X - 17354 | polyphenol related | 0.16 |
| X - 21286 | pyridine related | 0.14 |
| X - 12283 | aromatic amino acid related metabolite | 0.14 |
| X - 12738 | phenol-related | 0.14 |
| X - 24243 | piperidine related | 0.13 |
| X - 22520 | fatty acid conjugate | 0.13 |
| X - 11315 | amino acid derivative | 0.12 |
| X - 22509 | polyphenol related | 0.12 |
| X - 13844 | benzoic acid derivative | 0.1 |
| X - 13835 | aromatic amino acid related metabolite | 0.08 |

In Table 9, names of unknown compounds as provided by Metabolon Inc along with their new identification and candidate structures are provided. Microbiome R2 is the EV of each metabolite as estimated by a prediction model based on gut microbiome data

Networks of Interactions Between Features Explain Diverse Metabolites

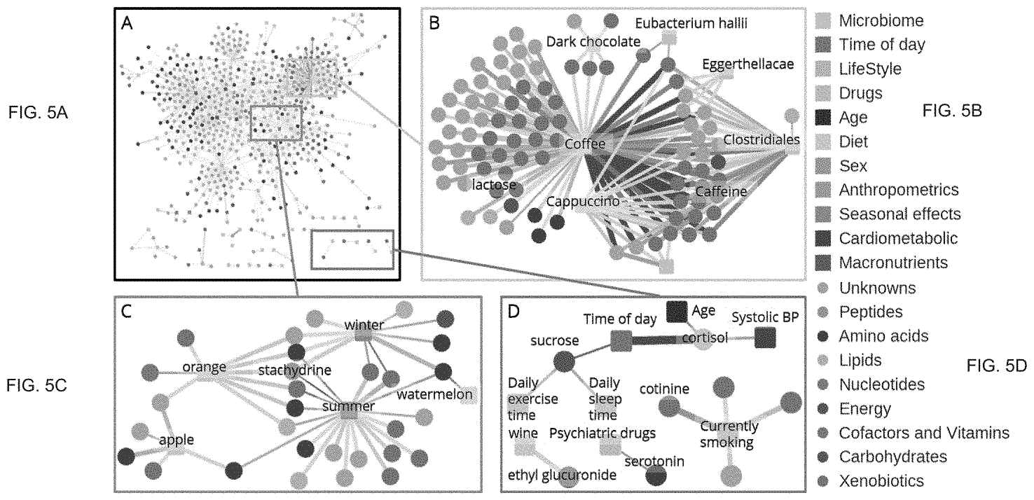

As multiple metabolites were significantly predicted using more than one feature group, we next examined how different feature groups interact in explaining the levels of these metabolites. By building separate predictive models each based on a different feature group and using SHAP in order to estimate the impact of each specific feature on the output of the models, we uncovered a dense network of interactions between feature groups in explaining metabolite levels (FIG. 5A).

As mentioned above, we found that the reported consumption of coffee was linked to a large number of metabolites, most of which are unknown compounds and xenobiotics from the xanthine metabolism pathway. Notably, we found that a specific bacterial species from the Clostridiales order was linked to a large number of these metabolites (FIG. 5B), suggesting a possible interaction between coffee consumption and the presence of this bacteria in explaining the levels of these metabolites. Being the most predictive features among their feature categories, coffee consumption and this Clostridiales species may be targets for validation using interventional studies.

We next focused on metabolites which were significantly explained using seasonal effects, and examined which dietary features interact with them (FIG. 5C). The consumption of citrus fruits such as oranges positively affected (on average) the prediction of several metabolites such as stachydrine, a known biomarker for the consumption of citrus fruits⁴⁵ (also named proline betaine; significantly predicted by diet, Pearson R=0.50, p<10⁻²⁰), which in turn had higher values in samples taken in winter months compared to samples taken during the summer, consistent with the fact that oranges are seasonal fruits available in Israel mostly during winter. Another example is N-methyltaurine (R=0.35, p<10⁻²⁰), an amino acid which has higher levels in samples taken during winter, and whose prediction was negatively affected, on average, by the consumption of watermelon, a summer seasonal fruit.

Finally, we explored some known examples of associations between metabolites and features to further validate the quality of data in our cohort (FIG. 5D). The diurnal cycle is known to regulate the levels of multiple circulating metabolites. We found that the levels of cortisol were lower in samples taken during the second half of the day (Prediction with time of day, R=0.63, p<10⁻²⁰, positive SHAP value for samples taken in the morning), consistent with previous studies showing that cortisol levels peak early in the morning⁴⁶. We also found that the levels of tobacco-related metabolites such as cotinine (Prediction R=0.72 by lifestyle, p<10⁻²⁰) were higher in samples of active smokers (positive SHAP values for smoking), and that no other feature could significantly explain their levels. Finally, we found that blood levels of serotonin (Prediction R=0.46 by drugs, p<10⁻⁶) were lower in samples of participants who reported taking psychiatric drugs (negative SHAP values), despite serotonin being a therapeutic target for selective serotonin reuptake inhibitors (SSRI)⁴⁷ which are prescribed to increase serotonin levels in the brain.

Metabolites Explained by Bread Increase Following a Bread Consumption Intervention

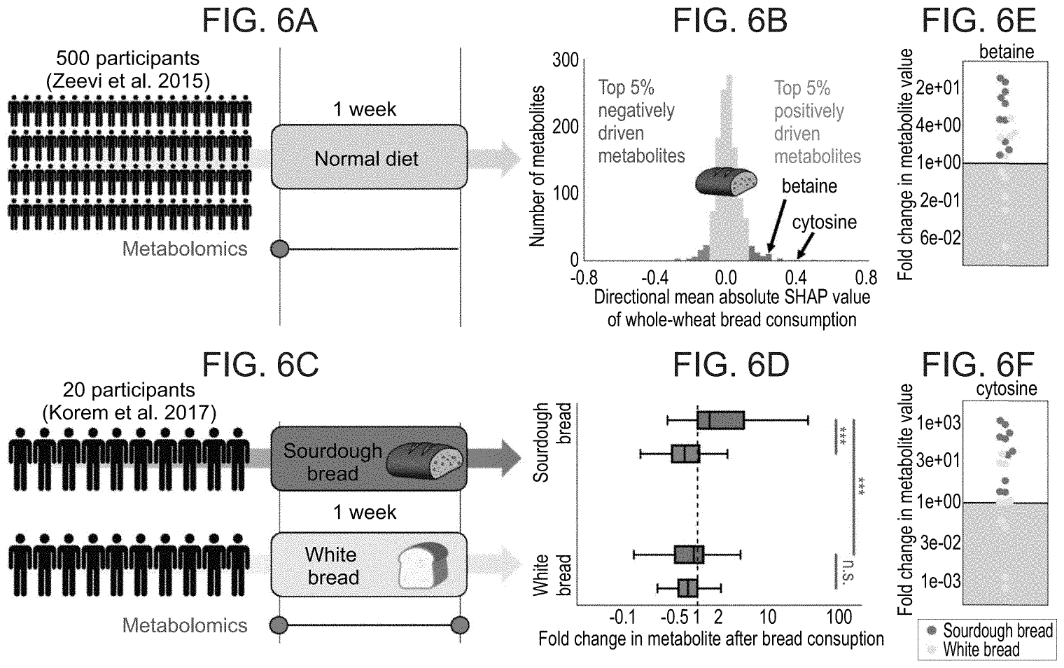

As a proof of concept examining whether some of the feature-metabolite interactions we uncovered may be causal, we profiled the serum metabolome of samples from a randomized cross-over trial that we previously conducted⁴⁸, in which we compared the effects of consuming artisanal whole-grain sourdough bread (hereinafter, “sourdough bread”) to those of industrial white bread made from refined wheat (“white bread”). Twenty healthy subjects were randomly divided into two groups of 10, who then underwent a 1-week-long dietary intervention of increased bread consumption, where each group received a different type of bread. Following two weeks of washout, the intervention was performed again, switching bread types between the groups. (FIG. 6C). In the present study, we performed metabolomic profiling of blood samples that were taken at both the beginning and the end of the first week of intervention, in order to estimate the effect of the dietary intervention on serum metabolites.

We used the healthy cohort of 458 participants for which we had one week of logged normal diet, without any intervention (FIG. 6A) to identify potential associations between the reported consumption of white and whole-wheat breads and the levels of metabolites (FIG. 6B). We ranked the metabolites according to the mean absolute SHAP value for consumption of whole-wheat bread computed based on the 458 participants, and selected the top 5% positively and negatively associated metabolites for further analysis (FIG. 6B). Notably, analyzing the metabolomic samples of subjects who received the sourdough bread intervention, we found that metabolites that were positively associated with the consumption of whole-wheat bread in our cohort increased significantly more (median fold-change 1.44) than metabolites that were negatively associated with the consumption of whole-wheat bread in the 458-participants cohort (median fold-change 0.66, p<10⁻⁸, Mann-Whitney U; FIG. 6D). Moreover, we found no statistically significant differences when comparing the mean fold-change of these metabolites in the group which received the white bread intervention (p>0.3, Mann-Whitney U; FIG. 6D).

Some of the metabolites which increased in levels following the sourdough bread intervention were previously reported to be linked to the consumption of whole-grain wheat flour. A notable example is betaine, an amino acid which has been shown to protect internal organs, improve vascular risk factors⁴⁹ and is also known to be highly abundant in a wide variety of foods, of which wheat bran and wheat germ are the highest naturally occurring sources^{50,51}. We found that in the group that received sourdough bread the mean fold-change in betaine levels was 6.16, while the mean fold-change in the group that received white bread was 0.82 (Mann-Whitney U p<0.004; FIG. 6E; Methods), consistent with the correlation between betaine levels and the consumption of whole-grain wheat in the larger cohort (Spearman R=0.14, p<0.003). Another example is cytosine, for which the mean fold-change was far greater in the sourdough bread compared to the white bread group, 78.5 vs. 0.53, respectively (Mann-Whitney U p<0.002; FIG. 6F). Unlike betaine, the levels of cytosine were not previously linked to the rate or type of bread consumption.

We also performed a similar analysis using metabolites that were associated with white bread consumption in our cohort, but did not find significant changes in these metabolites in the bread intervention study, potentially stemming from high white wheat consumption in the typical diet before the intervention. Overall, these results suggest that some of the associations that we found between the consumption of whole-wheat bread and the levels of metabolites in our larger cohort might be causal, as their levels increase following a dietary intervention that increased the consumption of whole-wheat bread.

### Sequence Identifiers for Metagenomic Sequences of Unknown Bacteria

Table 10 provides the sequence identifier for the metagenomic sequences of the unknown bacteria.

TABLE 10

| Seq ID | unknown bacteria number |
|---|---|
| 1 | 14921 |
| 2 | 13981 |
| 3 | 14252 |
| 4 | 4781 |
| 5 | 14999 |
| 6 | 14764 |
| 7 | 15385 |
| 8 | 4121 |
| 9 | 4121 |
| 10 | 14027 |
| 11 | 4121 |
| 12 | 4342 |
| 13 | 15403 |
| 14 | 13983 |
| 15 | 4029 |
| 16 | 4342 |
| 17 | 14999 |
| 18 | 4130 |
| 19 | 14250 |
| 20 | 4781 |
| 21 | 15390 |
| 22 | 14263 |
| 23 | 14250 |
| 24 | 15403 |
| 25 | 14999 |
| 26 | 14974 |
| 27 | 4029 |
| 28 | 4029 |
| 29 | 14921 |
| 30 | 14932 |
| 31 | 15403 |
| 32 | 4781 |
| 33 | 15403 |
| 34 | 13981 |
| 35 | 4342 |
| 36 | 15385 |
| 37 | 14263 |
| 38 | 8767 |
| 39 | 14263 |
| 40 | 14899 |
| 41 | 14921 |
| 42 | 3926 |
| 43 | 4121 |
| 44 | 14999 |
| 45 | 14999 |
| 46 | 14020 |
| 47 | 14252 |
| 48 | 14027 |
| 49 | 15403 |
| 50 | 14252 |
| 51 | 3964 |
| 52 | 14932 |
| 53 | 4395 |
| 54 | 14974 |
| 55 | 4130 |
| 56 | 14253 |
| 57 | 15403 |
| 58 | 15390 |
| 59 | 14999 |
| 60 | 14937 |
| 61 | 14252 |
| 62 | 14999 |
| 63 | 4342 |
| 64 | 4767 |
| 65 | 13982 |
| 66 | 4130 |
| 67 | 15350 |
| 68 | 14921 |
| 69 | 3574 |
| 70 | 3964 |
| 71 | 15395 |
| 72 | 14027 |
| 73 | 15403 |
| 74 | 4121 |
| 75 | 4121 |
| 76 | 14899 |
| 77 | 4394 |
| 78 | 4029 |
| 79 | 4781 |
| 80 | 4781 |
| 81 | 13983 |
| 82 | 14253 |
| 83 | 14921 |
| 84 | 8767 |
| 85 | 4781 |
| 86 | 14252 |
| 87 | 14252 |
| 88 | 15403 |
| 89 | 4029 |
| 90 | 4121 |
| 91 | 4029 |
| 92 | 15356 |
| 93 | 14999 |
| 94 | 4121 |
| 95 | 15390 |
| 96 | 15395 |
| 97 | 4781 |
| 98 | 8767 |
| 99 | 15403 |
| 100 | 4029 |
| 101 | 15403 |
| 102 | 15356 |
| 103 | 4029 |
| 104 | 15350 |
| 105 | 4781 |
| 106 | 14252 |
| 107 | 14764 |
| 108 | 4130 |
| 109 | 15403 |
| 110 | 15385 |
| 111 | 4130 |
| 112 | 14921 |
| 113 | 3574 |
| 114 | 14253 |
| 115 | 15403 |
| 116 | 4782 |
| 117 | 3926 |
| 118 | 4394 |
| 119 | 14937 |
| 120 | 14764 |
| 121 | 14252 |
| 122 | 15356 |
| 123 | 4029 |
| 124 | 14764 |
| 125 | 3952 |
| 126 | 15356 |
| 127 | 4342 |
| 128 | 4342 |
| 129 | 15403 |
| 130 | 14937 |
| 131 | 4029 |
| 132 | 15403 |
| 133 | 14861 |
| 134 | 8767 |
| 135 | 15350 |
| 136 | 3574 |
| 137 | 4130 |
| 138 | 13981 |
| 139 | 14999 |
| 140 | 14252 |
| 141 | 3940 |
| 142 | 3952 |
| 143 | 3926 |
| 144 | 15403 |
| 145 | 14252 |
| 146 | 14252 |
| 147 | 14252 |
| 148 | 14999 |
| 149 | 4767 |
| 150 | 4781 |
| 151 | 15403 |
| 152 | 14999 |
| 153 | 14250 |
| 154 | 14252 |
| 155 | 14252 |
| 156 | 14027 |
| 157 | 4130 |
| 158 | 4029 |
| 159 | 14999 |
| 160 | 14899 |
| 161 | 13981 |
| 162 | 4395 |
| 163 | 8767 |
| 164 | 14764 |
| 165 | 14252 |
| 166 | 3574 |
| 167 | 4029 |
| 168 | 14252 |
| 169 | 15350 |
| 170 | 14253 |
| 171 | 4767 |
| 172 | 4767 |
| 173 | 4130 |
| 174 | 14932 |
| 175 | 14764 |
| 176 | 14999 |
| 177 | 14253 |
| 178 | 4342 |
| 179 | 4342 |
| 180 | 3574 |
| 181 | 14999 |
| 182 | 14999 |
| 183 | 15403 |
| 184 | 13981 |
| 185 | 14921 |
| 186 | 4767 |
| 187 | 14921 |
| 188 | 4342 |
| 189 | 14921 |
| 190 | 14899 |
| 191 | 3926 |
| 192 | 4121 |
| 193 | 14252 |
| 194 | 14250 |
| 195 | 4394 |
| 196 | 4121 |
| 197 | 14999 |
| 198 | 4029 |
| 199 | 15390 |
| 200 | 15356 |
| 201 | 14974 |
| 202 | 14999 |
| 203 | 4121 |
| 204 | 14999 |
| 205 | 15403 |
| 206 | 15395 |
| 207 | 15385 |
| 208 | 4781 |
| 209 | 14899 |
| 210 | 14974 |
| 211 | 14252 |
| 212 | 4394 |
| 213 | 4781 |
| 214 | 4029 |
| 215 | 14999 |
| 216 | 14921 |
| 217 | 4394 |
| 218 | 4342 |
| 219 | 14252 |
| 220 | 14252 |
| 221 | 4121 |
| 222 | 3574 |
| 223 | 14253 |
| 224 | 3952 |
| 225 | 4394 |
| 226 | 4342 |
| 227 | 8767 |
| 228 | 15350 |
| 229 | 14027 |
| 230 | 3952 |
| 231 | 14252 |
| 232 | 3964 |
| 233 | 4121 |
| 234 | 14999 |
| 235 | 15356 |
| 236 | 4781 |
| 237 | 14937 |
| 238 | 4130 |
| 239 | 14999 |
| 240 | 14252 |
| 241 | 4342 |
| 242 | 14899 |
| 243 | 14974 |
| 244 | 14252 |
| 245 | 14932 |
| 246 | 14899 |
| 247 | 14253 |
| 248 | 14921 |
| 249 | 13981 |
| 250 | 15385 |
| 251 | 4342 |
| 252 | 14999 |
| 253 | 14250 |
| 254 | 14999 |
| 255 | 14764 |
| 256 | 15350 |
| 257 | 4782 |
| 258 | 14861 |
| 259 | 14253 |
| 260 | 3952 |
| 261 | 4394 |
| 262 | 4781 |
| 263 | 14252 |
| 264 | 14932 |
| 265 | 14252 |
| 266 | 4029 |
| 267 | 14764 |
| 268 | 3964 |
| 269 | 15395 |
| 270 | 15385 |
| 271 | 15403 |
| 272 | 4029 |
| 273 | 4029 |
| 274 | 4029 |
| 275 | 14899 |
| 276 | 14252 |
| 277 | 15403 |
| 278 | 14921 |
| 279 | 14250 |
| 280 | 3574 |
| 281 | 13982 |
| 282 | 14027 |
| 283 | 14974 |
| 284 | 3952 |
| 285 | 14999 |
| 286 | 15356 |
| 287 | 4342 |
| 288 | 4029 |
| 289 | 14252 |
| 290 | 14937 |
| 291 | 4781 |
| 292 | 15350 |
| 293 | 14999 |
| 294 | 14263 |
| 295 | 14899 |
| 296 | 14999 |
| 297 | 14999 |
| 298 | 14027 |
| 299 | 14921 |
| 300 | 14252 |
| 301 | 3926 |
| 302 | 14999 |
| 303 | 4342 |
| 304 | 14764 |
| 305 | 4029 |
| 306 | 14253 |
| 307 | 3940 |
| 308 | 15356 |
| 309 | 14764 |
| 310 | 13981 |
| 311 | 14899 |
| 312 | 14899 |
| 313 | 15395 |
| 314 | 4342 |
| 315 | 14764 |
| 316 | 15403 |
| 317 | 4029 |
| 318 | 3964 |
| 319 | 14921 |
| 320 | 4781 |
| 321 | 14764 |
| 322 | 4029 |
| 323 | 3940 |
| 324 | 14252 |
| 325 | 14253 |
| 326 | 4342 |
| 327 | 14999 |
| 328 | 15356 |
| 329 | 14999 |
| 330 | 4342 |
| 331 | 15403 |
| 332 | 3574 |
| 333 | 14999 |
| 334 | 4342 |
| 335 | 14999 |
| 336 | 14252 |
| 337 | 3952 |
| 338 | 14921 |
| 339 | 14932 |
| 340 | 15403 |
| 341 | 15350 |
| 342 | 4342 |
| 343 | 3952 |
| 344 | 14252 |
| 345 | 4029 |
| 346 | 14252 |
| 347 | 14252 |
| 348 | 14974 |
| 349 | 4029 |
| 350 | 14999 |
| 351 | 14253 |
| 352 | 13981 |
| 353 | 3952 |
| 354 | 14921 |
| 355 | 14764 |
| 356 | 15403 |
| 357 | 14252 |
| 358 | 14974 |
| 359 | 15390 |
| 360 | 15390 |
| 361 | 3574 |
| 362 | 4394 |
| 363 | 14899 |
| 364 | 14252 |
| 365 | 14764 |
| 366 | 14764 |
| 367 | 3940 |
| 368 | 14999 |
| 369 | 13981 |
| 370 | 4781 |
| 371 | 4029 |
| 372 | 14027 |
| 373 | 13981 |
| 374 | 14932 |
| 375 | 14899 |
| 376 | 13981 |
| 377 | 14252 |
| 378 | 15403 |
| 379 | 15395 |
| 380 | 15350 |
| 381 | 4342 |
| 382 | 14899 |
| 383 | 4395 |
| 384 | 4029 |
| 385 | 13981 |
| 386 | 14263 |
| 387 | 14253 |
| 388 | 3574 |
| 389 | 13981 |
| 390 | 14252 |
| 391 | 14999 |
| 392 | 14921 |
| 393 | 15403 |
| 394 | 4342 |
| 395 | 4342 |
| 396 | 4029 |
| 397 | 14252 |
| 398 | 4342 |
| 399 | 3574 |
| 400 | 4121 |
| 401 | 14999 |
| 402 | 14764 |
| 403 | 4029 |
| 404 | 14252 |
| 405 | 4782 |
| 406 | 14764 |
| 407 | 3952 |
| 408 | 14861 |
| 409 | 14899 |
| 410 | 13982 |
| 411 | 14999 |
| 412 | 14999 |
| 413 | 4781 |
| 414 | 15385 |
| 415 | 14999 |
| 416 | 4782 |
| 417 | 13981 |
| 418 | 14937 |
| 419 | 3940 |
| 420 | 4029 |
| 421 | 15350 |
| 422 | 4342 |
| 423 | 4121 |
| 424 | 4767 |
| 425 | 3940 |
| 426 | 14921 |
| 427 | 3964 |
| 428 | 3964 |
| 429 | 13981 |
| 430 | 15350 |
| 431 | 14252 |
| 432 | 4767 |
| 433 | 15350 |
| 434 | 14764 |
| 435 | 4121 |
| 436 | 14252 |
| 437 | 4781 |
| 438 | 14253 |
| 439 | 4394 |
| 440 | 14899 |
| 441 | 14999 |
| 442 | 14999 |
| 443 | 14921 |
| 444 | 4781 |
| 445 | 14999 |
| 446 | 5184 |
| 447 | 4342 |
| 448 | 14027 |
| 449 | 14999 |
| 450 | 13981 |
| 451 | 14764 |
| 452 | 14932 |
| 453 | 14764 |
| 454 | 13981 |
| 455 | 3574 |
| 456 | 3964 |
| 457 | 13982 |
| 458 | 3574 |
| 459 | 4781 |
| 460 | 4781 |
| 461 | 4782 |
| 462 | 14252 |
| 463 | 3952 |
| 464 | 15403 |
| 465 | 15390 |
| 466 | 14252 |
| 467 | 14250 |
| 468 | 14764 |
| 469 | 14999 |
| 470 | 4342 |
| 471 | 3952 |
| 472 | 13981 |
| 473 | 14999 |
| 474 | 14027 |
| 475 | 14999 |
| 476 | 3964 |
| 477 | 3574 |
| 478 | 14250 |
| 479 | 3574 |
| 480 | 4121 |
| 481 | 8767 |
| 482 | 14999 |
| 483 | 15350 |
| 484 | 14899 |
| 485 | 4782 |
| 486 | 15390 |
| 487 | 3952 |
| 488 | 14974 |
| 489 | 14764 |
| 490 | 4394 |
| 491 | 13981 |
| 492 | 14974 |
| 493 | 4342 |
| 494 | 4781 |
| 495 | 15403 |
| 496 | 15385 |
| 497 | 14932 |
| 498 | 14764 |
| 499 | 14253 |
| 500 | 4130 |
| 501 | 3952 |
| 502 | 14252 |
| 503 | 14253 |
| 504 | 4029 |
| 505 | 3940 |
| 506 | 14999 |
| 507 | 14899 |
| 508 | 14253 |
| 509 | 3574 |
| 510 | 14252 |
| 511 | 14252 |
| 512 | 14999 |
| 513 | 4029 |
| 514 | 14999 |
| 515 | 4767 |
| 516 | 14252 |
| 517 | 4342 |
| 518 | 4029 |
| 519 | 13981 |
| 520 | 14252 |
| 521 | 13983 |
| 522 | 14999 |
| 523 | 3964 |
| 524 | 15403 |
| 525 | 4342 |
| 526 | 14252 |
| 527 | 14252 |
| 528 | 3574 |
| 529 | 15390 |
| 530 | 14764 |
| 531 | 14764 |
| 532 | 14253 |
| 533 | 14999 |
| 534 | 15403 |
| 535 | 4395 |
| 536 | 14253 |
| 537 | 14020 |
| 538 | 4342 |
| 539 | 14899 |
| 540 | 14252 |
| 541 | 3940 |
| 542 | 14921 |
| 543 | 14250 |
| 544 | 15395 |
| 545 | 15385 |
| 546 | 14999 |
| 547 | 14999 |
| 548 | 4029 |
| 549 | 14937 |
| 550 | 14764 |
| 551 | 4130 |
| 552 | 3926 |
| 553 | 14764 |
| 554 | 14250 |
| 555 | 4782 |
| 556 | 14252 |
| 557 | 15385 |
| 558 | 14250 |
| 559 | 14974 |
| 560 | 15385 |
| 561 | 14974 |
| 562 | 4130 |
| 563 | 14253 |
| 564 | 14899 |
| 565 | 4767 |
| 566 | 14899 |
| 567 | 4121 |
| 568 | 14921 |
| 569 | 14252 |
| 570 | 3964 |
| 571 | 14252 |
| 572 | 4767 |
| 573 | 4121 |
| 574 | 14921 |
| 575 | 14764 |
| 576 | 13981 |
| 577 | 14999 |
| 578 | 14921 |
| 579 | 4782 |
| 580 | 14764 |
| 581 | 15395 |
| 582 | 14921 |
| 583 | 15403 |
| 584 | 14252 |
| 585 | 4781 |
| 586 | 14921 |
| 587 | 14764 |
| 588 | 14999 |
| 589 | 14921 |
| 590 | 13983 |
| 591 | 14921 |
| 592 | 15350 |
| 593 | 14932 |
| 594 | 14764 |
| 595 | 15385 |
| 596 | 3574 |
| 597 | 4781 |
| 598 | 4767 |
| 599 | 14899 |
| 600 | 3964 |
| 601 | 4342 |
| 602 | 13981 |
| 603 | 14921 |
| 604 | 13982 |
| 605 | 14999 |
| 606 | 14974 |
| 607 | 14932 |
| 608 | 4029 |
| 609 | 15385 |
| 610 | 15350 |
| 611 | 4767 |
| 612 | 4130 |
| 613 | 14263 |
| 614 | 14252 |
| 615 | 4782 |
| 616 | 4781 |
| 617 | 14252 |
| 618 | 4767 |
| 619 | 14252 |
| 620 | 4029 |
| 621 | 14921 |
| 622 | 13982 |
| 623 | 3926 |
| 624 | 14999 |
| 625 | 14999 |
| 626 | 15403 |
| 627 | 4782 |
| 628 | 3952 |
| 629 | 4121 |
| 630 | 14252 |
| 631 | 14764 |
| 632 | 14937 |
| 633 | 3574 |
| 634 | 4394 |
| 635 | 15403 |
| 636 | 4342 |
| 637 | 4767 |
| 638 | 3574 |
| 639 | 14250 |
| 640 | 14764 |
| 641 | 3574 |
| 642 | 15395 |
| 643 | 15356 |
| 644 | 14764 |
| 645 | 13981 |
| 646 | 4121 |
| 647 | 4394 |
| 648 | 14861 |
| 649 | 4130 |
| 650 | 14921 |
| 651 | 4029 |
| 652 | 14252 |
| 653 | 14020 |
| 654 | 14250 |
| 655 | 3574 |
| 656 | 15356 |
| 657 | 14921 |
| 658 | 15356 |
| 659 | 14999 |
| 660 | 14937 |
| 661 | 3574 |
| 662 | 3574 |
| 663 | 4029 |
| 664 | 4342 |
| 665 | 4781 |
| 666 | 3574 |
| 667 | 15403 |
| 668 | 14999 |
| 669 | 14764 |
| 670 | 4782 |
| 671 | 3574 |
| 672 | 4130 |
| 673 | 14899 |
| 674 | 4342 |
| 675 | 4781 |
| 676 | 14253 |
| 677 | 15385 |
| 678 | 4781 |
| 679 | 4029 |
| 680 | 14921 |
| 681 | 14253 |
| 682 | 13981 |
| 683 | 14252 |
| 684 | 14899 |
| 685 | 14974 |
| 686 | 14252 |
| 687 | 14899 |
| 688 | 3574 |
| 689 | 14252 |
| 690 | 4394 |
| 691 | 14921 |
| 692 | 8767 |
| 693 | 14263 |
| 694 | 15395 |
| 695 | 3964 |
| 696 | 14027 |
| 697 | 3940 |
| 698 | 15403 |
| 699 | 3940 |
| 700 | 14921 |
| 701 | 3964 |
| 702 | 14899 |
| 703 | 14764 |
| 704 | 15403 |
| 705 | 14999 |
| 706 | 3964 |
| 707 | 4781 |
| 708 | 14253 |
| 709 | 14999 |
| 710 | 4342 |
| 711 | 15350 |
| 712 | 4342 |
| 713 | 5184 |
| 714 | 4121 |
| 715 | 4342 |
| 716 | 4029 |
| 717 | 4029 |
| 718 | 14932 |
| 719 | 4767 |
| 720 | 3926 |
| 721 | 15403 |
| 722 | 15403 |
| 723 | 13981 |
| 724 | 14764 |
| 725 | 14764 |
| 726 | 4029 |
| 727 | 15403 |
| 728 | 14252 |
| 729 | 14764 |
| 730 | 3964 |
| 731 | 14921 |
| 732 | 4342 |
| 733 | 4029 |
| 734 | 15403 |
| 735 | 3940 |
| 736 | 4781 |
| 737 | 14253 |
| 738 | 15385 |
| 739 | 14999 |
| 740 | 4781 |
| 741 | 4029 |
| 742 | 4342 |
| 743 | 14027 |
| 744 | 15403 |
| 745 | 15395 |
| 746 | 14999 |
| 747 | 14899 |
| 748 | 4782 |
| 749 | 3926 |
| 750 | 15395 |
| 751 | 14999 |
| 752 | 14899 |
| 753 | 4342 |
| 754 | 4029 |
| 755 | 4342 |
| 756 | 14027 |
| 757 | 14937 |
| 758 | 3952 |
| 759 | 14899 |
| 760 | 14921 |
| 761 | 13981 |
| 762 | 14250 |
| 763 | 15390 |
| 764 | 14999 |
| 765 | 15356 |
| 766 | 3574 |
| 767 | 15350 |
| 768 | 4029 |
| 769 | 15403 |
| 770 | 3574 |
| 771 | 14764 |
| 772 | 14252 |
| 773 | 14974 |
| 774 | 14252 |
| 775 | 5184 |
| 776 | 15403 |
| 777 | 4130 |
| 778 | 3964 |
| 779 | 3574 |
| 780 | 14027 |
| 781 | 4121 |
| 782 | 3952 |
| 783 | 4029 |
| 784 | 3574 |
| 785 | 4781 |
| 786 | 14253 |
| 787 | 14999 |
| 788 | 4767 |
| 789 | 14252 |
| 790 | 14921 |
| 791 | 4395 |
| 792 | 15356 |
| 793 | 13983 |
| 794 | 14999 |
| 795 | 15403 |
| 796 | 4782 |
| 797 | 3964 |
| 798 | 3574 |
| 799 | 14252 |
| 800 | 14861 |
| 801 | 3964 |
| 802 | 15403 |
| 803 | 14999 |
| 804 | 15403 |
| 805 | 13981 |
| 806 | 4029 |
| 807 | 14020 |
| 808 | 14027 |
| 809 | 14764 |
| 810 | 14252 |
| 811 | 3574 |
| 812 | 4781 |
| 813 | 14764 |
| 814 | 3926 |
| 815 | 14999 |
| 816 | 3574 |
| 817 | 14250 |
| 818 | 13981 |
| 819 | 4342 |
| 820 | 3574 |
| 821 | 14974 |
| 822 | 14252 |
| 823 | 4342 |
| 824 | 15350 |
| 825 | 3574 |
| 826 | 15350 |
| 827 | 14999 |
| 828 | 14764 |
| 829 | 3574 |
| 830 | 14764 |
| 831 | 15350 |
| 832 | 4029 |
| 833 | 3940 |
| 834 | 3952 |
| 835 | 14250 |
| 836 | 14921 |
| 837 | 14999 |
| 838 | 4767 |
| 839 | 4781 |
| 840 | 4342 |
| 841 | 4029 |
| 842 | 15395 |
| 843 | 15403 |
| 844 | 13981 |
| 845 | 4342 |
| 846 | 3952 |
| 847 | 13982 |
| 848 | 14932 |
| 849 | 14974 |
| 850 | 15403 |
| 851 | 15356 |
| 852 | 3574 |
| 853 | 3940 |
| 854 | 14250 |
| 855 | 14899 |
| 856 | 14999 |
| 857 | 14861 |
| 858 | 14937 |
| 859 | 14250 |
| 860 | 14974 |
| 861 | 14999 |
| 862 | 14027 |
| 863 | 15385 |
| 864 | 14764 |
| 865 | 4130 |
| 866 | 13981 |
| 867 | 15395 |
| 868 | 4767 |
| 869 | 13981 |
| 870 | 3926 |
| 871 | 15350 |
| 872 | 15385 |
| 873 | 4781 |
| 874 | 4394 |
| 875 | 4029 |
| 876 | 14027 |
| 877 | 4781 |
| 878 | 4781 |
| 879 | 14764 |
| 880 | 14253 |
| 881 | 4029 |
| 882 | 4342 |
| 883 | 5184 |
| 884 | 3574 |
| 885 | 3940 |
| 886 | 3940 |
| 887 | 14999 |
| 888 | 14921 |
| 889 | 15356 |
| 890 | 4342 |
| 891 | 15385 |
| 892 | 15390 |
| 893 | 14252 |
| 894 | 14764 |
| 895 | 14764 |
| 896 | 15356 |
| 897 | 4029 |
| 898 | 3926 |
| 899 | 15385 |
| 900 | 14921 |
| 901 | 3952 |
| 902 | 15403 |
| 903 | 14999 |
| 904 | 15403 |
| 905 | 14999 |
| 906 | 14899 |
| 907 | 4394 |
| 908 | 4395 |
| 909 | 14764 |
| 910 | 14252 |
| 911 | 14252 |
| 912 | 4782 |
| 913 | 14252 |
| 914 | 14899 |
| 915 | 15350 |
| 916 | 8767 |
| 917 | 14252 |
| 918 | 14974 |
| 919 | 14999 |
| 920 | 3574 |
| 921 | 3940 |
| 922 | 14999 |
| 923 | 14252 |
| 924 | 14252 |
| 925 | 15403 |
| 926 | 14899 |
| 927 | 14252 |
| 928 | 3940 |
| 929 | 14252 |
| 930 | 14937 |
| 931 | 14253 |
| 932 | 14764 |
| 933 | 15395 |
| 934 | 3574 |
| 935 | 4781 |
| 936 | 3574 |
| 937 | 14252 |
| 938 | 15350 |
| 939 | 15385 |
| 940 | 3964 |
| 941 | 14252 |
| 942 | 14027 |
| 943 | 14921 |
| 944 | 3940 |
| 945 | 15350 |
| 946 | 14999 |
| 947 | 4342 |
| 948 | 15403 |
| 949 | 14027 |
| 950 | 4029 |
| 951 | 14899 |
| 952 | 3574 |
| 953 | 4767 |
| 954 | 14921 |
| 955 | 14999 |
| 956 | 14250 |
| 957 | 14764 |
| 958 | 14764 |
| 959 | 14999 |
| 960 | 14999 |
| 961 | 13981 |
| 962 | 15385 |
| 963 | 4781 |
| 964 | 14764 |
| 965 | 15403 |
| 966 | 4029 |
| 967 | 14250 |
| 968 | 4781 |
| 969 | 14764 |
| 970 | 14974 |
| 971 | 14764 |
| 972 | 15350 |
| 973 | 3574 |
| 974 | 4781 |
| 975 | 4767 |
| 976 | 3574 |
| 977 | 4781 |
| 978 | 14921 |
| 979 | 4781 |
| 980 | 14999 |
| 981 | 14937 |
| 982 | 14027 |
| 983 | 4121 |
| 984 | 14252 |
| 985 | 4394 |
| 986 | 4767 |
| 987 | 15350 |
| 988 | 4767 |
| 989 | 4781 |
| 990 | 14027 |
| 991 | 4121 |
| 992 | 15403 |
| 993 | 3964 |
| 994 | 4342 |
| 995 | 14932 |
| 996 | 4767 |
| 997 | 14252 |
| 998 | 14999 |
| 999 | 4029 |
| 1000 | 14899 |
| 1001 | 4781 |
| 1002 | 4394 |
| 1003 | 4781 |
| 1004 | 14921 |
| 1005 | 4130 |
| 1006 | 4342 |
| 1007 | 4782 |
| 1008 | 14027 |
| 1009 | 8767 |
| 1010 | 14974 |
| 1011 | 14764 |
| 1012 | 4029 |
| 1013 | 15403 |
| 1014 | 3940 |
| 1015 | 4767 |
| 1016 | 3964 |
| 1017 | 4394 |
| 1018 | 4342 |
| 1019 | 3964 |
| 1020 | 15356 |
| 1021 | 14974 |
| 1022 | 14027 |
| 1023 | 14999 |
| 1024 | 14252 |
| 1025 | 4781 |
| 1026 | 4029 |
| 1027 | 4781 |
| 1028 | 3940 |
| 1029 | 14252 |
| 1030 | 4394 |
| 1031 | 14252 |
| 1032 | 14999 |
| 1033 | 14764 |
| 1034 | 4767 |
| 1035 | 14999 |
| 1036 | 15356 |
| 1037 | 3964 |
| 1038 | 14999 |
| 1039 | 4342 |
| 1040 | 15403 |
| 1041 | 15403 |
| 1042 | 14253 |
| 1043 | 14764 |
| 1044 | 14020 |
| 1045 | 14253 |
| 1046 | 15385 |
| 1047 | 4342 |
| 1048 | 14263 |
| 1049 | 15356 |
| 1050 | 14252 |
| 1051 | 14932 |
| 1052 | 4029 |
| 1053 | 13981 |
| 1054 | 3574 |
| 1055 | 4029 |
| 1056 | 4342 |
| 1057 | 14764 |
| 1058 | 4029 |
| 1059 | 14252 |
| 1060 | 4029 |
| 1061 | 14921 |
| 1062 | 4394 |
| 1063 | 3574 |
| 1064 | 3940 |
| 1065 | 14974 |
| 1066 | 15350 |
| 1067 | 4781 |
| 1068 | 15403 |
| 1069 | 15403 |
| 1070 | 8767 |
| 1071 | 14999 |
| 1072 | 14999 |
| 1073 | 14974 |
| 1074 | 4342 |
| 1075 | 15395 |
| 1076 | 3926 |
| 1077 | 14921 |
| 1078 | 4342 |
| 1079 | 14764 |
| 1080 | 14921 |
| 1081 | 4029 |
| 1082 | 4781 |
| 1083 | 4767 |
| 1084 | 14921 |
| 1085 | 3926 |
| 1086 | 14263 |
| 1087 | 14921 |
| 1088 | 14253 |
| 1089 | 4767 |
| 1090 | 14020 |
| 1091 | 4029 |
| 1092 | 4029 |
| 1093 | 5184 |
| 1094 | 14921 |
| 1095 | 3574 |
| 1096 | 14899 |
| 1097 | 14921 |
| 1098 | 15403 |
| 1099 | 14253 |
| 1100 | 14250 |
| 1101 | 4394 |
| 1102 | 4394 |
| 1103 | 3964 |
| 1104 | 4342 |
| 1105 | 8767 |
| 1106 | 15385 |
| 1107 | 4029 |
| 1108 | 14921 |
| 1109 | 13982 |
| 1110 | 3574 |
| 1111 | 14250 |
| 1112 | 14999 |
| 1113 | 15395 |
| 1114 | 4394 |
| 1115 | 15395 |
| 1116 | 4342 |
| 1117 | 4342 |
| 1118 | 4781 |
| 1119 | 14252 |
| 1120 | 14253 |
| 1121 | 4781 |
| 1122 | 3574 |
| 1123 | 14252 |
| 1124 | 3964 |
| 1125 | 4029 |
| 1126 | 15350 |
| 1127 | 14999 |
| 1128 | 4394 |
| 1129 | 14764 |
| 1130 | 14899 |
| 1131 | 14250 |
| 1132 | 4121 |
| 1133 | 4781 |
| 1134 | 3964 |
| 1135 | 3926 |
| 1136 | 15390 |
| 1137 | 3574 |
| 1138 | 14253 |
| 1139 | 14932 |
| 1140 | 14999 |
| 1141 | 14899 |
| 1142 | 14027 |
| 1143 | 4029 |
| 1144 | 14020 |
| 1145 | 14764 |
| 1146 | 14899 |
| 1147 | 3952 |
| 1148 | 14764 |
| 1149 | 14921 |
| 1150 | 14932 |
| 1151 | 4767 |
| 1152 | 4342 |
| 1153 | 14252 |
| 1154 | 3964 |
| 1155 | 15403 |
| 1156 | 13981 |
| 1157 | 13981 |
| 1158 | 3952 |
| 1159 | 15356 |
| 1160 | 4781 |
| 1161 | 14252 |
| 1162 | 14899 |
| 1163 | 14921 |
| 1164 | 14974 |
| 1165 | 14921 |
| 1166 | 14921 |
| 1167 | 4121 |
| 1168 | 3952 |
| 1169 | 14764 |
| 1170 | 15385 |
| 1171 | 14899 |
| 1172 | 5184 |
| 1173 | 3574 |
| 1174 | 14921 |
| 1175 | 4130 |
| 1176 | 3940 |
| 1177 | 14252 |
| 1178 | 14999 |
| 1179 | 14899 |
| 1180 | 14899 |
| 1181 | 14027 |
| 1182 | 14999 |
| 1183 | 14764 |
| 1184 | 15350 |
| 1185 | 4029 |
| 1186 | 4029 |
| 1187 | 14999 |
| 1188 | 14020 |
| 1189 | 14999 |
| 1190 | 15356 |
| 1191 | 14999 |
| 1192 | 14764 |
| 1193 | 3574 |
| 1194 | 13981 |
| 1195 | 3574 |
| 1196 | 4781 |
| 1197 | 4130 |
| 1198 | 14253 |
| 1199 | 4121 |
| 1200 | 4130 |
| 1201 | 14252 |
| 1202 | 15356 |
| 1203 | 3574 |
| 1204 | 14921 |
| 1205 | 15395 |
| 1206 | 15395 |
| 1207 | 8767 |
| 1208 | 4029 |
| 1209 | 14252 |
| 1210 | 4781 |
| 1211 | 3964 |
| 1212 | 14921 |
| 1213 | 15403 |
| 1214 | 4781 |
| 1215 | 14027 |
| 1216 | 4342 |
| 1217 | 15356 |
| 1218 | 14999 |
| 1219 | 3952 |
| 1220 | 4029 |
| 1221 | 4781 |
| 1222 | 4342 |
| 1223 | 14252 |
| 1224 | 14899 |
| 1225 | 3940 |
| 1226 | 3952 |
| 1227 | 14999 |
| 1228 | 14027 |
| 1229 | 4130 |
| 1230 | 3964 |
| 1231 | 14999 |
| 1232 | 14764 |
| 1233 | 3574 |
| 1234 | 4781 |
| 1235 | 14921 |
| 1236 | 14252 |
| 1237 | 14999 |
| 1238 | 15385 |
| 1239 | 4342 |
| 1240 | 14921 |
| 1241 | 4781 |
| 1242 | 14937 |
| 1243 | 14899 |
| 1244 | 8767 |
| 1245 | 4781 |
| 1246 | 3964 |
| 1247 | 3964 |
| 1248 | 3952 |
| 1249 | 15390 |
| 1250 | 4781 |
| 1251 | 4342 |
| 1252 | 4781 |
| 1253 | 14764 |
| 1254 | 4781 |
| 1255 | 14252 |
| 1256 | 15350 |
| 1257 | 4342 |
| 1258 | 14899 |
| 1259 | 4342 |
| 1260 | 15385 |
| 1261 | 14899 |
| 1262 | 15385 |
| 1263 | 14764 |
| 1264 | 14999 |
| 1265 | 3952 |
| 1266 | 14252 |
| 1267 | 14999 |
| 1268 | 14921 |
| 1269 | 15385 |
| 1270 | 4130 |
| 1271 | 4029 |
| 1272 | 14252 |
| 1273 | 14999 |
| 1274 | 15385 |
| 1275 | 4781 |
| 1276 | 14921 |
| 1277 | 4342 |
| 1278 | 4781 |
| 1279 | 14253 |
| 1280 | 14764 |
| 1281 | 14764 |
| 1282 | 4394 |
| 1283 | 15390 |
| 1284 | 14764 |
| 1285 | 15403 |
| 1286 | 5184 |
| 1287 | 4781 |
| 1288 | 4394 |
| 1289 | 14932 |
| 1290 | 14252 |
| 1291 | 4781 |
| 1292 | 14252 |
| 1293 | 4029 |
| 1294 | 15350 |
| 1295 | 4782 |
| 1296 | 4029 |
| 1297 | 15356 |
| 1298 | 4029 |
| 1299 | 15403 |
| 1300 | 14999 |
| 1301 | 4130 |
| 1302 | 3940 |
| 1303 | 14252 |
| 1304 | 14999 |
| 1305 | 4342 |
| 1306 | 3926 |
| 1307 | 14764 |
| 1308 | 4781 |
| 1309 | 3574 |
| 1310 | 4767 |
| 1311 | 14764 |
| 1312 | 15403 |
| 1313 | 4342 |
| 1314 | 15403 |
| 1315 | 3940 |
| 1316 | 15356 |
| 1317 | 14921 |
| 1318 | 4029 |
| 1319 | 14921 |
| 1320 | 4781 |
| 1321 | 14253 |
| 1322 | 14252 |
| 1323 | 4121 |
| 1324 | 14921 |
| 1325 | 4342 |
| 1326 | 5184 |
| 1327 | 15403 |
| 1328 | 4782 |
| 1329 | 14999 |
| 1330 | 13981 |
| 1331 | 4029 |
| 1332 | 14263 |
| 1333 | 15395 |
| 1334 | 4029 |
| 1335 | 4781 |
| 1336 | 8767 |
| 1337 | 4767 |
| 1338 | 4767 |
| 1339 | 15356 |
| 1340 | 15356 |
| 1341 | 4394 |
| 1342 | 14252 |
| 1343 | 14999 |
| 1344 | 14253 |
| 1345 | 4130 |
| 1346 | 14999 |
| 1347 | 14253 |
| 1348 | 3952 |
| 1349 | 4029 |
| 1350 | 15356 |
| 1351 | 4029 |
| 1352 | 14253 |
| 1353 | 4767 |
| 1354 | 4130 |
| 1355 | 14252 |
| 1356 | 4130 |
| 1357 | 15395 |
| 1358 | 3926 |
| 1359 | 15390 |
| 1360 | 14932 |
| 1361 | 15356 |
| 1362 | 4342 |
| 1363 | 13981 |
| 1364 | 4781 |
| 1365 | 14252 |
| 1366 | 15356 |
| 1367 | 4394 |
| 1368 | 4782 |
| 1369 | 4767 |
| 1370 | 4029 |
| 1371 | 15350 |
| 1372 | 15395 |
| 1373 | 4395 |
| 1374 | 4782 |
| 1375 | 4029 |
| 1376 | 4782 |
| 1377 | 14921 |
| 1378 | 13981 |
| 1379 | 3952 |
| 1380 | 4342 |
| 1381 | 4767 |
| 1382 | 14999 |
| 1383 | 14764 |
| 1384 | 13981 |
| 1385 | 3574 |
| 1386 | 14999 |
| 1387 | 4342 |
| 1388 | 14899 |
| 1389 | 3574 |
| 1390 | 4342 |
| 1391 | 4342 |
| 1392 | 14999 |
| 1393 | 15356 |
| 1394 | 4029 |
| 1395 | 3952 |
| 1396 | 14899 |
| 1397 | 4394 |
| 1398 | 3952 |
| 1399 | 4029 |
| 1400 | 13982 |
| 1401 | 14921 |
| 1402 | 14999 |
| 1403 | 4029 |
| 1404 | 14999 |
| 1405 | 4342 |
| 1406 | 14253 |
| 1407 | 4781 |
| 1408 | 5184 |
| 1409 | 4782 |
| 1410 | 4394 |
| 1411 | 14020 |
| 1412 | 4029 |
| 1413 | 14899 |
| 1414 | 14027 |
| 1415 | 3940 |
| 1416 | 8767 |
| 1417 | 3940 |
| 1418 | 14263 |
| 1419 | 4029 |
| 1420 | 3574 |
| 1421 | 14252 |
| 1422 | 15395 |
| 1423 | 4029 |
| 1424 | 4342 |
| 1425 | 4029 |
| 1426 | 4394 |
| 1427 | 14999 |
| 1428 | 15395 |
| 1429 | 13982 |
| 1430 | 4029 |
| 1431 | 4767 |
| 1432 | 14263 |
| 1433 | 14252 |
| 1434 | 4781 |
| 1435 | 3952 |
| 1436 | 15395 |
| 1437 | 4342 |
| 1438 | 14764 |
| 1439 | 14253 |
| 1440 | 4781 |
| 1441 | 3574 |
| 1442 | 4342 |
| 1443 | 3926 |
| 1444 | 14921 |
| 1445 | 14764 |
| 1446 | 14861 |
| 1447 | 14999 |
| 1448 | 14253 |
| 1449 | 4781 |
| 1450 | 14974 |
| 1451 | 15403 |
| 1452 | 4394 |
| 1453 | 4767 |
| 1454 | 15403 |
| 1455 | 4342 |
| 1456 | 3964 |
| 1457 | 14764 |
| 1458 | 15350 |
| 1459 | 14999 |
| 1460 | 15395 |
| 1461 | 14921 |
| 1462 | 3574 |
| 1463 | 4029 |
| 1464 | 14974 |
| 1465 | 4782 |
| 1466 | 4342 |
| 1467 | 14252 |
| 1468 | 3574 |
| 1469 | 14974 |
| 1470 | 14252 |
| 1471 | 14764 |
| 1472 | 4394 |
| 1473 | 4781 |
| 1474 | 4781 |
| 1475 | 4029 |
| 1476 | 14899 |
| 1477 | 14764 |
| 1478 | 14252 |
| 1479 | 14027 |
| 1480 | 14861 |
| 1481 | 4767 |
| 1482 | 15350 |
| 1483 | 15403 |
| 1484 | 14999 |
| 1485 | 14252 |
| 1486 | 3940 |
| 1487 | 14921 |
| 1488 | 4130 |
| 1489 | 14999 |
| 1490 | 4342 |
| 1491 | 14974 |
| 1492 | 4342 |
| 1493 | 4394 |
| 1494 | 14999 |
| 1495 | 14253 |
| 1496 | 4395 |
| 1497 | 4395 |
| 1498 | 13982 |
| 1499 | 4342 |
| 1500 | 14974 |
| 1501 | 14974 |
| 1502 | 3574 |
| 1503 | 13982 |
| 1504 | 14921 |
| 1505 | 4394 |
| 1506 | 14263 |
| 1507 | 4394 |
| 1508 | 15385 |
| 1509 | 15356 |
| 1510 | 14932 |
| 1511 | 13981 |
| 1512 | 15403 |
| 1513 | 13983 |
| 1514 | 4130 |
| 1515 | 15385 |
| 1516 | 14263 |
| 1517 | 4781 |
| 1518 | 4767 |
| 1519 | 3940 |
| 1520 | 14252 |
| 1521 | 14921 |
| 1522 | 14764 |
| 1523 | 14899 |
| 1524 | 15350 |
| 1525 | 15395 |
| 1526 | 15385 |
| 1527 | 4029 |
| 1528 | 15385 |
| 1529 | 4394 |
| 1530 | 14974 |
| 1531 | 15385 |
| 1532 | 4781 |
| 1533 | 15385 |
| 1534 | 4395 |
| 1535 | 4029 |
| 1536 | 14921 |
| 1537 | 3940 |
| 1538 | 14921 |
| 1539 | 14937 |
| 1540 | 14027 |
| 1541 | 15390 |
| 1542 | 3574 |
| 1543 | 4781 |
| 1544 | 14921 |
| 1545 | 14764 |
| 1546 | 4029 |
| 1547 | 15390 |
| 1548 | 15385 |
| 1549 | 14253 |
| 1550 | 13983 |
| 1551 | 15390 |
| 1552 | 15350 |
| 1553 | 14999 |
| 1554 | 4395 |
| 1555 | 4781 |
| 1556 | 14999 |
| 1557 | 14999 |
| 1558 | 14999 |
| 1559 | 4130 |
| 1560 | 14921 |
| 1561 | 14921 |
| 1562 | 15350 |
| 1563 | 14252 |
| 1564 | 15385 |
| 1565 | 4029 |
| 1566 | 8767 |
| 1567 | 4782 |
| 1568 | 4781 |
| 1569 | 15390 |
| 1570 | 14974 |
| 1571 | 4342 |
| 1572 | 14921 |
| 1573 | 4029 |
| 1574 | 14764 |
| 1575 | 4781 |
| 1576 | 4395 |
| 1577 | 15403 |
| 1578 | 14764 |
| 1579 | 4121 |
| 1580 | 14020 |
| 1581 | 14937 |
| 1582 | 4342 |
| 1583 | 14921 |
| 1584 | 4394 |
| 1585 | 4781 |
| 1586 | 4130 |
| 1587 | 14999 |
| 1588 | 14899 |
| 1589 | 13981 |
| 1590 | 4782 |
| 1591 | 14937 |
| 1592 | 15350 |
| 1593 | 14921 |
| 1594 | 4342 |
| 1595 | 3574 |
| 1596 | 4767 |
| 1597 | 4342 |
| 1598 | 14999 |
| 1599 | 4781 |
| 1600 | 14921 |
| 1601 | 14921 |
| 1602 | 4029 |
| 1603 | 4029 |
| 1604 | 4781 |
| 1605 | 14764 |
| 1606 | 3940 |
| 1607 | 4342 |
| 1608 | 14252 |
| 1609 | 3574 |
| 1610 | 14764 |
| 1611 | 13982 |
| 1612 | 15395 |
| 1613 | 4781 |
| 1614 | 4394 |
| 1615 | 15385 |
| 1616 | 14974 |
| 1617 | 14999 |
| 1618 | 15395 |
| 1619 | 14999 |
| 1620 | 13981 |
| 1621 | 14252 |
| 1622 | 14764 |
| 1623 | 15395 |
| 1624 | 14764 |
| 1625 | 15356 |
| 1626 | 14764 |
| 1627 | 15403 |
| 1628 | 14253 |
| 1629 | 3940 |
| 1630 | 14764 |
| 1631 | 15395 |
| 1632 | 4782 |
| 1633 | 14020 |
| 1634 | 4767 |
| 1635 | 15385 |
| 1636 | 4781 |
| 1637 | 14263 |
| 1638 | 14974 |
| 1639 | 14921 |
| 1640 | 14937 |
| 1641 | 14252 |
| 1642 | 14921 |
| 1643 | 14250 |
| 1644 | 14252 |
| 1645 | 14263 |
| 1646 | 14921 |
| 1647 | 14250 |
| 1648 | 14764 |
| 1649 | 4767 |
| 1650 | 15395 |
| 1651 | 14250 |
| 1652 | 4029 |
| 1653 | 14999 |
| 1654 | 15356 |
| 1655 | 3964 |
| 1656 | 15403 |
| 1657 | 13981 |
| 1658 | 14764 |
| 1659 | 15395 |
| 1660 | 4767 |
| 1661 | 4781 |
| 1662 | 3952 |
| 1663 | 4394 |
| 1664 | 4121 |
| 1665 | 13981 |
| 1666 | 3574 |
| 1667 | 14253 |
| 1668 | 14252 |
| 1669 | 4029 |
| 1670 | 4029 |
| 1671 | 3952 |
| 1672 | 14252 |
| 1673 | 15403 |
| 1674 | 14764 |
| 1675 | 4029 |
| 1676 | 3574 |
| 1677 | 15356 |
| 1678 | 14027 |
| 1679 | 14921 |
| 1680 | 3940 |
| 1681 | 14263 |
| 1682 | 14250 |
| 1683 | 13981 |
| 1684 | 14250 |
| 1685 | 14253 |
| 1686 | 14937 |
| 1687 | 14921 |
| 1688 | 4781 |
| 1689 | 14250 |
| 1690 | 14253 |
| 1691 | 15350 |
| 1692 | 14253 |
| 1693 | 14999 |
| 1694 | 13981 |
| 1695 | 4342 |
| 1696 | 14921 |
| 1697 | 4029 |
| 1698 | 14899 |
| 1699 | 3574 |
| 1700 | 3940 |
| 1701 | 4394 |
| 1702 | 14999 |
| 1703 | 15350 |
| 1704 | 14253 |
| 1705 | 4342 |
| 1706 | 4342 |
| 1707 | 3952 |
| 1708 | 4781 |
| 1709 | 15356 |
| 1710 | 14937 |
| 1711 | 14253 |
| 1712 | 14899 |
| 1713 | 4767 |
| 1714 | 4342 |
| 1715 | 4121 |
| 1716 | 4342 |
| 1717 | 4029 |
| 1718 | 14999 |
| 1719 | 14974 |
| 1720 | 14899 |
| 1721 | 15385 |
| 1722 | 4130 |
| 1723 | 3952 |
| 1724 | 4395 |
| 1725 | 4782 |
| 1726 | 4130 |
| 1727 | 14932 |
| 1728 | 3574 |
| 1729 | 4342 |
| 1730 | 14764 |
| 1731 | 13981 |
| 1732 | 4029 |
| 1733 | 4767 |
| 1734 | 14027 |
| 1735 | 14263 |
| 1736 | 14263 |
| 1737 | 4029 |
| 1738 | 14252 |
| 1739 | 4130 |
| 1740 | 13981 |
| 1741 | 14252 |
| 1742 | 4342 |
| 1743 | 3926 |
| 1744 | 13981 |
| 1745 | 4029 |
| 1746 | 14921 |
| 1747 | 14764 |
| 1748 | 14932 |
| 1749 | 14921 |
| 1750 | 14764 |
| 1751 | 14252 |
| 1752 | 4130 |
| 1753 | 13981 |
| 1754 | 14252 |
| 1755 | 14020 |
| 1756 | 3940 |
| 1757 | 13981 |
| 1758 | 14764 |
| 1759 | 15403 |
| 1760 | 14027 |
| 1761 | 15403 |
| 1762 | 14999 |
| 1763 | 15403 |
| 1764 | 14899 |
| 1765 | 4767 |
| 1766 | 14921 |
| 1767 | 14764 |
| 1768 | 4130 |
| 1769 | 13981 |
| 1770 | 14999 |
| 1771 | 14764 |
| 1772 | 4130 |
| 1773 | 14027 |
| 1774 | 15350 |
| 1775 | 3952 |
| 1776 | 14020 |
| 1777 | 14937 |
| 1778 | 14937 |
| 1779 | 14252 |
| 1780 | 15403 |
| 1781 | 15350 |
| 1782 | 14252 |
| 1783 | 14999 |
| 1784 | 4782 |
| 1785 | 13981 |
| 1786 | 14263 |
| 1787 | 14974 |
| 1788 | 14921 |
| 1789 | 14027 |
| 1790 | 14974 |
| 1791 | 4767 |
| 1792 | 14999 |
| 1793 | 4029 |
| 1794 | 14263 |
| 1795 | 4029 |
| 1796 | 14899 |
| 1797 | 4029 |
| 1798 | 14252 |
| 1799 | 14764 |
| 1800 | 14252 |
| 1801 | 14253 |
| 1802 | 4029 |
| 1803 | 15395 |
| 1804 | 4781 |
| 1805 | 4029 |
| 1806 | 14027 |
| 1807 | 3926 |
| 1808 | 14252 |
| 1809 | 14921 |
| 1810 | 14921 |
| 1811 | 15390 |
| 1812 | 14253 |
| 1813 | 14253 |
| 1814 | 14252 |
| 1815 | 4121 |
| 1816 | 3574 |
| 1817 | 4781 |
| 1818 | 14252 |
| 1819 | 3574 |
| 1820 | 15350 |
| 1821 | 4342 |
| 1822 | 15403 |
| 1823 | 13982 |
| 1824 | 14252 |
| 1825 | 14253 |
| 1826 | 3574 |
| 1827 | 3964 |
| 1828 | 3952 |
| 1829 | 13983 |
| 1830 | 4029 |
| 1831 | 4029 |
| 1832 | 8767 |
| 1833 | 14999 |
| 1834 | 15350 |
| 1835 | 4782 |
| 1836 | 3964 |
| 1837 | 14020 |
| 1838 | 4342 |
| 1839 | 14020 |
| 1840 | 4395 |
| 1841 | 14250 |
| 1842 | 14764 |
| 1843 | 14899 |
| 1844 | 13981 |
| 1845 | 4782 |
| 1846 | 14921 |
| 1847 | 4781 |
| 1848 | 14999 |
| 1849 | 3574 |
| 1850 | 3940 |
| 1851 | 4781 |
| 1852 | 4342 |
| 1853 | 14252 |
| 1854 | 3574 |
| 1855 | 4767 |
| 1856 | 14764 |
| 1857 | 14252 |
| 1858 | 14252 |
| 1859 | 4029 |
| 1860 | 3952 |
| 1861 | 4342 |
| 1862 | 3952 |
| 1863 | 14253 |
| 1864 | 4394 |
| 1865 | 4781 |
| 1866 | 15356 |
| 1867 | 4029 |
| 1868 | 14027 |
| 1869 | 14921 |
| 1870 | 15395 |
| 1871 | 4342 |
| 1872 | 14999 |
| 1873 | 14999 |
| 1874 | 4342 |
| 1875 | 8767 |
| 1876 | 4781 |
| 1877 | 4342 |
| 1878 | 4767 |
| 1879 | 14899 |
| 1880 | 14921 |
| 1881 | 4029 |
| 1882 | 4767 |
| 1883 | 14253 |
| 1884 | 4767 |
| 1885 | 14932 |
| 1886 | 4029 |
| 1887 | 3964 |
| 1888 | 15385 |
| 1889 | 14027 |
| 1890 | 4781 |
| 1891 | 4781 |
| 1892 | 4342 |
| 1893 | 14252 |
| 1894 | 4767 |
| 1895 | 14999 |
| 1896 | 4029 |
| 1897 | 14764 |
| 1898 | 14764 |
| 1899 | 4342 |
| 1900 | 14764 |
| 1901 | 3574 |
| 1902 | 3964 |
| 1903 | 14252 |
| 1904 | 14937 |
| 1905 | 14999 |
| 1906 | 3574 |
| 1907 | 13981 |
| 1908 | 14764 |
| 1909 | 14252 |
| 1910 | 4029 |
| 1911 | 3952 |
| 1912 | 3574 |
| 1913 | 4781 |
| 1914 | 13983 |
| 1915 | 14764 |
| 1916 | 4394 |
| 1917 | 4130 |
| 1918 | 3574 |
| 1919 | 14974 |
| 1920 | 14921 |
| 1921 | 4029 |
| 1922 | 4782 |
| 1923 | 4029 |
| 1924 | 3952 |
| 1925 | 4029 |
| 1926 | 14252 |
| 1927 | 3574 |
| 1928 | 14252 |
| 1929 | 13981 |
| 1930 | 14253 |
| 1931 | 4781 |
| 1932 | 3574 |
| 1933 | 14899 |
| 1934 | 4394 |
| 1935 | 13981 |
| 1936 | 14999 |
| 1937 | 15356 |
| 1938 | 14899 |
| 1939 | 14252 |
| 1940 | 15395 |
| 1941 | 3574 |
| 1942 | 3926 |
| 1943 | 4342 |
| 1944 | 3574 |
| 1945 | 4342 |
| 1946 | 4029 |
| 1947 | 15356 |
| 1948 | 4342 |
| 1949 | 4767 |
| 1950 | 4029 |
| 1951 | 14027 |
| 1952 | 4121 |
| 1953 | 3964 |
| 1954 | 4781 |
| 1955 | 14764 |
| 1956 | 14252 |
| 1957 | 3574 |
| 1958 | 4767 |
| 1959 | 4781 |
| 1960 | 15350 |
| 1961 | 15385 |
| 1962 | 15385 |
| 1963 | 13982 |
| 1964 | 4130 |
| 1965 | 3574 |
| 1966 | 4029 |
| 1967 | 14932 |
| 1968 | 14764 |
| 1969 | 14250 |
| 1970 | 14999 |
| 1971 | 3952 |
| 1972 | 14252 |
| 1973 | 14899 |
| 1974 | 4342 |
| 1975 | 14999 |
| 1976 | 4782 |
| 1977 | 14764 |
| 1978 | 14899 |
| 1979 | 14921 |
| 1980 | 15385 |
| 1981 | 15350 |
| 1982 | 14921 |
| 1983 | 14932 |
| 1984 | 4342 |
| 1985 | 4781 |
| 1986 | 4121 |
| 1987 | 15403 |
| 1988 | 4342 |
| 1989 | 15350 |
| 1990 | 4394 |
| 1991 | 14764 |
| 1992 | 13981 |
| 1993 | 4130 |
| 1994 | 14252 |
| 1995 | 15356 |
| 1996 | 14899 |
| 1997 | 14999 |
| 1998 | 14999 |
| 1999 | 14999 |
| 2000 | 3574 |
| 2001 | 14999 |
| 2002 | 14921 |
| 2003 | 3964 |
| 2004 | 3574 |
| 2005 | 3574 |
| 2006 | 14250 |
| 2007 | 14899 |
| 2008 | 14999 |
| 2009 | 4781 |
| 2010 | 4394 |
| 2011 | 4781 |
| 2012 | 4782 |
| 2013 | 14921 |
| 2014 | 15385 |
| 2015 | 15385 |
| 2016 | 4130 |
| 2017 | 3940 |
| 2018 | 14263 |
| 2019 | 14932 |
| 2020 | 14252 |
| 2021 | 14764 |
| 2022 | 4342 |
| 2023 | 14921 |
| 2024 | 14899 |
| 2025 | 13981 |
| 2026 | 14921 |
| 2027 | 14252 |
| 2028 | 14899 |
| 2029 | 3574 |
| 2030 | 4767 |
| 2031 | 3952 |
| 2032 | 14764 |
| 2033 | 14999 |
| 2034 | 4130 |
| 2035 | 3574 |
| 2036 | 14027 |
| 2037 | 4029 |
| 2038 | 14253 |
| 2039 | 15403 |
| 2040 | 3964 |
| 2041 | 14252 |
| 2042 | 14899 |
| 2043 | 8767 |
| 2044 | 14252 |
| 2045 | 4781 |
| 2046 | 14899 |
| 2047 | 15403 |
| 2048 | 3964 |
| 2049 | 14999 |
| 2050 | 14999 |
| 2051 | 3574 |
| 2052 | 14999 |
| 2053 | 4767 |
| 2054 | 14921 |
| 2055 | 14252 |
| 2056 | 14253 |
| 2057 | 15385 |
| 2058 | 5184 |
| 2059 | 4781 |
| 2060 | 14020 |
| 2061 | 13983 |
| 2062 | 4781 |
| 2063 | 14974 |
| 2064 | 4029 |
| 2065 | 14999 |
| 2066 | 14252 |
| 2067 | 14027 |
| 2068 | 14250 |
| 2069 | 4781 |
| 2070 | 15356 |
| 2071 | 14253 |
| 2072 | 4394 |
| 2073 | 14764 |
| 2074 | 4342 |
| 2075 | 14263 |
| 2076 | 14999 |
| 2077 | 8767 |
| 2078 | 13981 |
| 2079 | 4781 |
| 2080 | 4342 |
| 2081 | 3964 |
| 2082 | 14252 |
| 2083 | 4767 |
| 2084 | 15390 |
| 2085 | 15390 |
| 2086 | 14250 |
| 2087 | 3574 |
| 2088 | 13981 |
| 2089 | 4767 |
| 2090 | 15385 |
| 2091 | 14999 |
| 2092 | 4782 |
| 2093 | 3964 |
| 2094 | 4782 |
| 2095 | 4767 |
| 2096 | 3574 |
| 2097 | 3940 |
| 2098 | 3574 |
| 2099 | 14921 |
| 2100 | 14921 |
| 2101 | 3940 |
| 2102 | 14937 |
| 2103 | 14899 |
| 2104 | 14921 |
| 2105 | 4782 |
| 2106 | 14861 |
| 2107 | 14932 |
| 2108 | 4029 |
| 2109 | 4130 |
| 2110 | 14252 |
| 2111 | 14253 |
| 2112 | 4130 |
| 2113 | 14974 |
| 2114 | 15356 |
| 2115 | 14250 |
| 2116 | 4781 |
| 2117 | 14974 |
| 2118 | 14253 |
| 2119 | 4767 |
| 2120 | 13981 |
| 2121 | 14921 |
| 2122 | 3964 |
| 2123 | 14999 |
| 2124 | 4781 |
| 2125 | 14899 |
| 2126 | 15403 |
| 2127 | 14263 |
| 2128 | 15385 |
| 2129 | 14921 |
| 2130 | 14921 |
| 2131 | 14899 |
| 2132 | 4781 |
| 2133 | 4121 |
| 2134 | 4342 |
| 2135 | 14764 |
| 2136 | 4121 |
| 2137 | 4781 |
| 2138 | 14764 |
| 2139 | 14253 |
| 2140 | 14999 |
| 2141 | 14253 |
| 2142 | 4767 |
| 2143 | 15356 |
| 2144 | 4029 |
| 2145 | 4029 |
| 2146 | 3574 |
| 2147 | 15385 |
| 2148 | 4130 |
| 2149 | 3926 |
| 2150 | 14263 |
| 2151 | 14020 |
| 2152 | 14899 |
| 2153 | 3574 |
| 2154 | 4029 |
| 2155 | 14764 |
| 2156 | 14250 |
| 2157 | 3952 |
| 2158 | 4781 |
| 2159 | 4342 |
| 2160 | 14921 |
| 2161 | 14921 |
| 2162 | 3964 |
| 2163 | 14252 |
| 2164 | 15403 |
| 2165 | 13981 |
| 2166 | 14252 |
| 2167 | 14253 |
| 2168 | 3952 |
| 2169 | 14999 |
| 2170 | 4342 |
| 2171 | 14252 |
| 2172 | 15385 |
| 2173 | 4342 |
| 2174 | 14764 |
| 2175 | 3940 |
| 2176 | 8767 |
| 2177 | 14937 |
| 2178 | 13981 |
| 2179 | 4767 |
| 2180 | 14921 |
| 2181 | 15395 |
| 2182 | 14921 |
| 2183 | 14764 |
| 2184 | 14253 |
| 2185 | 4781 |
| 2186 | 4781 |
| 2187 | 4029 |
| 2188 | 15403 |
| 2189 | 4342 |
| 2190 | 14999 |
| 2191 | 14999 |
| 2192 | 15390 |
| 2193 | 15385 |
| 2194 | 3574 |
| 2195 | 3574 |
| 2196 | 14921 |
| 2197 | 4781 |
| 2198 | 15356 |
| 2199 | 14932 |
| 2200 | 4342 |
| 2201 | 14764 |
| 2202 | 14027 |
| 2203 | 4781 |
| 2204 | 14999 |
| 2205 | 4394 |
| 2206 | 14027 |
| 2207 | 4029 |
| 2208 | 14764 |
| 2209 | 15356 |
| 2210 | 3940 |
| 2211 | 4767 |
| 2212 | 14027 |
| 2213 | 14974 |
| 2214 | 3964 |
| 2215 | 4029 |
| 2216 | 14764 |
| 2217 | 4130 |
| 2218 | 4781 |
| 2219 | 4767 |
| 2220 | 13982 |
| 2221 | 15390 |
| 2222 | 14899 |
| 2223 | 14027 |
| 2224 | 4121 |
| 2225 | 14253 |
| 2226 | 4121 |
| 2227 | 4394 |
| 2228 | 13982 |
| 2229 | 4394 |
| 2230 | 4782 |
| 2231 | 4394 |
| 2232 | 14921 |
| 2233 | 3964 |
| 2234 | 14974 |
| 2235 | 15356 |
| 2236 | 4029 |
| 2237 | 14253 |
| 2238 | 4781 |
| 2239 | 3952 |
| 2240 | 4782 |
| 2241 | 14921 |
| 2242 | 14999 |
| 2243 | 4781 |
| 2244 | 14999 |
| 2245 | 14999 |
| 2246 | 4029 |
| 2247 | 14861 |
| 2248 | 14027 |
| 2249 | 4342 |
| 2250 | 14974 |
| 2251 | 4121 |
| 2252 | 13981 |
| 2253 | 3926 |
| 2254 | 14252 |
| 2255 | 4029 |
| 2256 | 3940 |
| 2257 | 14252 |
| 2258 | 4394 |
| 2259 | 14921 |
| 2260 | 4342 |
| 2261 | 4767 |
| 2262 | 14932 |
| 2263 | 14999 |
| 2264 | 14921 |
| 2265 | 4029 |
| 2266 | 4342 |
| 2267 | 15390 |
| 2268 | 4130 |
| 2269 | 15385 |
| 2270 | 14764 |
| 2271 | 4029 |
| 2272 | 3574 |
| 2273 | 4130 |
| 2274 | 4394 |
| 2275 | 15403 |
| 2276 | 14974 |
| 2277 | 8767 |
| 2278 | 4342 |
| 2279 | 14252 |
| 2280 | 13982 |
| 2281 | 4782 |
| 2282 | 15385 |
| 2283 | 14764 |
| 2284 | 14252 |
| 2285 | 3574 |
| 2286 | 14253 |
| 2287 | 14253 |
| 2288 | 4029 |
| 2289 | 4767 |
| 2290 | 3952 |
| 2291 | 4029 |
| 2292 | 14252 |
| 2293 | 14932 |
| 2294 | 3964 |
| 2295 | 14764 |
| 2296 | 14921 |
| 2297 | 15385 |
| 2298 | 14764 |
| 2299 | 14999 |
| 2300 | 14253 |
| 2301 | 14263 |
| 2302 | 14252 |
| 2303 | 14899 |
| 2304 | 4029 |
| 2305 | 4767 |
| 2306 | 14263 |
| 2307 | 3952 |
| 2308 | 15385 |
| 2309 | 14899 |
| 2310 | 13981 |
| 2311 | 14999 |
| 2312 | 15403 |
| 2313 | 14937 |
| 2314 | 4342 |
| 2315 | 14250 |
| 2316 | 4130 |
| 2317 | 14253 |
| 2318 | 14921 |
| 2319 | 4394 |
| 2320 | 4781 |
| 2321 | 4029 |
| 2322 | 14899 |
| 2323 | 3574 |
| 2324 | 14252 |
| 2325 | 14921 |
| 2326 | 14921 |
| 2327 | 15403 |
| 2328 | 14252 |
| 2329 | 14999 |
| 2330 | 14921 |
| 2331 | 14252 |
| 2332 | 4029 |
| 2333 | 15385 |
| 2334 | 14921 |
| 2335 | 14899 |
| 2336 | 13981 |
| 2337 | 14764 |
| 2338 | 15395 |
| 2339 | 14974 |
| 2340 | 3574 |
| 2341 | 4130 |
| 2342 | 4781 |
| 2343 | 15385 |
| 2344 | 14899 |
| 2345 | 15403 |
| 2346 | 15385 |
| 2347 | 14252 |
| 2348 | 14921 |
| 2349 | 14921 |
| 2350 | 14250 |
| 2351 | 4394 |
| 2352 | 14899 |
| 2353 | 14921 |
| 2354 | 14027 |
| 2355 | 15385 |
| 2356 | 14764 |
| 2357 | 15395 |
| 2358 | 15395 |
| 2359 | 14252 |
| 2360 | 13983 |
| 2361 | 4395 |
| 2362 | 3940 |
| 2363 | 4767 |
| 2364 | 4029 |
| 2365 | 4121 |
| 2366 | 14999 |
| 2367 | 15390 |
| 2368 | 4781 |
| 2369 | 4029 |
| 2370 | 14921 |
| 2371 | 15403 |
| 2372 | 4394 |
| 2373 | 4781 |
| 2374 | 3952 |
| 2375 | 13981 |
| 2376 | 14252 |
| 2377 | 14253 |
| 2378 | 15403 |
| 2379 | 14764 |
| 2380 | 14921 |
| 2381 | 4121 |
| 2382 | 14937 |
| 2383 | 4121 |
| 2384 | 14764 |
| 2385 | 14999 |
| 2386 | 14764 |
| 2387 | 4029 |
| 2388 | 14999 |
| 2389 | 4029 |
| 2390 | 14999 |
| 2391 | 4781 |
| 2392 | 4767 |
| 2393 | 4781 |
| 2394 | 14921 |
| 2395 | 3952 |
| 2396 | 4781 |
| 2397 | 14252 |
| 2398 | 4130 |
| 2399 | 13981 |
| 2400 | 3952 |
| 2401 | 4394 |
| 2402 | 4767 |
| 2403 | 14899 |
| 2404 | 3574 |
| 2405 | 13982 |
| 2406 | 14253 |
| 2407 | 3964 |
| 2408 | 14999 |
| 2409 | 3574 |
| 2410 | 15403 |
| 2411 | 14253 |
| 2412 | 15385 |
| 2413 | 4767 |
| 2414 | 4029 |
| 2415 | 15385 |
| 2416 | 14027 |
| 2417 | 5184 |
| 2418 | 14937 |
| 2419 | 3926 |
| 2420 | 14937 |
| 2421 | 4781 |
| 2422 | 3964 |
| 2423 | 14921 |
| 2424 | 15356 |
| 2425 | 4342 |
| 2426 | 14252 |
| 2427 | 14764 |
| 2428 | 15356 |
| 2429 | 14252 |
| 2430 | 4029 |
| 2431 | 3926 |
| 2432 | 4342 |
| 2433 | 14250 |
| 2434 | 14921 |
| 2435 | 4029 |
| 2436 | 4781 |
| 2437 | 3574 |
| 2438 | 3574 |
| 2439 | 3940 |
| 2440 | 3952 |
| 2441 | 3574 |
| 2442 | 4342 |
| 2443 | 4029 |
| 2444 | 4121 |
| 2445 | 3940 |
| 2446 | 14253 |
| 2447 | 3574 |
| 2448 | 3952 |
| 2449 | 3964 |
| 2450 | 14252 |
| 2451 | 14764 |
| 2452 | 14937 |
| 2453 | 4029 |
| 2454 | 8767 |
| 2455 | 13981 |
| 2456 | 4394 |
| 2457 | 15350 |
| 2458 | 15350 |
| 2459 | 14974 |
| 2460 | 14937 |
| 2461 | 14921 |
| 2462 | 4130 |
| 2463 | 14999 |
| 2464 | 13981 |
| 2465 | 15350 |
| 2466 | 14974 |
| 2467 | 4029 |
| 2468 | 14932 |
| 2469 | 14250 |
| 2470 | 3574 |
| 2471 | 14250 |
| 2472 | 14764 |
| 2473 | 4781 |
| 2474 | 4121 |
| 2475 | 14764 |
| 2476 | 4029 |
| 2477 | 14921 |
| 2478 | 4342 |
| 2479 | 3964 |
| 2480 | 14999 |
| 2481 | 15385 |
| 2482 | 14937 |
| 2483 | 4767 |
| 2484 | 3574 |
| 2485 | 8767 |
| 2486 | 15395 |
| 2487 | 14027 |
| 2488 | 14899 |
| 2489 | 14899 |
| 2490 | 13983 |
| 2491 | 3574 |
| 2492 | 14921 |
| 2493 | 14263 |
| 2494 | 15403 |
| 2495 | 14253 |
| 2496 | 14020 |
| 2497 | 3574 |
| 2498 | 14263 |
| 2499 | 13981 |
| 2500 | 4121 |
| 2501 | 15385 |
| 2502 | 14253 |
| 2503 | 14921 |
| 2504 | 15390 |
| 2505 | 5184 |
| 2506 | 4342 |
| 2507 | 15390 |
| 2508 | 13983 |
| 2509 | 14250 |
| 2510 | 14999 |
| 2511 | 3574 |
| 2512 | 4781 |
| 2513 | 13981 |
| 2514 | 14974 |
| 2515 | 14937 |
| 2516 | 14764 |
| 2517 | 14027 |
| 2518 | 14932 |
| 2519 | 14764 |
| 2520 | 15395 |
| 2521 | 14974 |
| 2522 | 14999 |
| 2523 | 15385 |
| 2524 | 14764 |
| 2525 | 4130 |
| 2526 | 14027 |
| 2527 | 4121 |
| 2528 | 14899 |
| 2529 | 4121 |
| 2530 | 4130 |
| 2531 | 4342 |
| 2532 | 4121 |
| 2533 | 4130 |
| 2534 | 3964 |
| 2535 | 4394 |
| 2536 | 14861 |
| 2537 | 4342 |
| 2538 | 4767 |
| 2539 | 14999 |
| 2540 | 14027 |
| 2541 | 13981 |
| 2542 | 8767 |
| 2543 | 14921 |
| 2544 | 15403 |
| 2545 | 4767 |
| 2546 | 14253 |
| 2547 | 14932 |
| 2548 | 14999 |
| 2549 | 14764 |
| 2550 | 3574 |
| 2551 | 13982 |
| 2552 | 3952 |
| 2553 | 4394 |
| 2554 | 14027 |
| 2555 | 15385 |
| 2556 | 14921 |
| 2557 | 3952 |
| 2558 | 14974 |
| 2559 | 15403 |
| 2560 | 8767 |
| 2561 | 15350 |
| 2562 | 15390 |
| 2563 | 14253 |
| 2564 | 4029 |
| 2565 | 14252 |
| 2566 | 13981 |
| 2567 | 4029 |
| 2568 | 3964 |
| 2569 | 3940 |
| 2570 | 4767 |
| 2571 | 14764 |
| 2572 | 3574 |
| 2573 | 4767 |
| 2574 | 15350 |
| 2575 | 4781 |
| 2576 | 14921 |
| 2577 | 14764 |
| 2578 | 15403 |
| 2579 | 14899 |
| 2580 | 14899 |
| 2581 | 15350 |
| 2582 | 4781 |
| 2583 | 3926 |
| 2584 | 13981 |
| 2585 | 3926 |
| 2586 | 3964 |
| 2587 | 14253 |
| 2588 | 4395 |
| 2589 | 13982 |
| 2590 | 14252 |
| 2591 | 13981 |
| 2592 | 4342 |
| 2593 | 14253 |
| 2594 | 4394 |
| 2595 | 14921 |
| 2596 | 4130 |
| 2597 | 4029 |
| 2598 | 3926 |
| 2599 | 14899 |
| 2600 | 15356 |
| 2601 | 14263 |
| 2602 | 4029 |
| 2603 | 14999 |
| 2604 | 14974 |
| 2605 | 4782 |
| 2606 | 3964 |
| 2607 | 14999 |
| 2608 | 4395 |
| 2609 | 4781 |
| 2610 | 14764 |
| 2611 | 4394 |
| 2612 | 14027 |
| 2613 | 14764 |
| 2614 | 14999 |
| 2615 | 4029 |
| 2616 | 13981 |
| 2617 | 14252 |
| 2618 | 14253 |
| 2619 | 14764 |
| 2620 | 14921 |
| 2621 | 4394 |
| 2622 | 4342 |
| 2623 | 4029 |
| 2624 | 4342 |
| 2625 | 14999 |
| 2626 | 4342 |
| 2627 | 15385 |
| 2628 | 14937 |
| 2629 | 14764 |
| 2630 | 14921 |
| 2631 | 14253 |
| 2632 | 14252 |
| 2633 | 14764 |
| 2634 | 15395 |
| 2635 | 14932 |
| 2636 | 4394 |
| 2637 | 14252 |
| 2638 | 14999 |
| 2639 | 3964 |
| 2640 | 14253 |
| 2641 | 14999 |
| 2642 | 14999 |
| 2643 | 15395 |
| 2644 | 15356 |
| 2645 | 4029 |
| 2646 | 14932 |
| 2647 | 14937 |
| 2648 | 4767 |
| 2649 | 4781 |
| 2650 | 15385 |
| 2651 | 14921 |
| 2652 | 15390 |
| 2653 | 13981 |
| 2654 | 14937 |
| 2655 | 13981 |
| 2656 | 4781 |
| 2657 | 14974 |
| 2658 | 15350 |
| 2659 | 14899 |
| 2660 | 14253 |
| 2661 | 14764 |
| 2662 | 3574 |
| 2663 | 8767 |
| 2664 | 4781 |
| 2665 | 14764 |
| 2666 | 14253 |
| 2667 | 3940 |
| 2668 | 15356 |
| 2669 | 14999 |
| 2670 | 3574 |
| 2671 | 4342 |
| 2672 | 4342 |
| 2673 | 4342 |
| 2674 | 14764 |
| 2675 | 4029 |
| 2676 | 4029 |
| 2677 | 4394 |
| 2678 | 14250 |
| 2679 | 14899 |
| 2680 | 5184 |
| 2681 | 4029 |
| 2682 | 14921 |
| 2683 | 14999 |
| 2684 | 14937 |
| 2685 | 15403 |
| 2686 | 14999 |
| 2687 | 15385 |
| 2688 | 3926 |
| 2689 | 14263 |
| 2690 | 4029 |
| 2691 | 14899 |
| 2692 | 14999 |
| 2693 | 15395 |
| 2694 | 14764 |
| 2695 | 14899 |
| 2696 | 15390 |
| 2697 | 14764 |
| 2698 | 14999 |
| 2699 | 4342 |
| 2700 | 3574 |
| 2701 | 15385 |
| 2702 | 14999 |
| 2703 | 4767 |
| 2704 | 15403 |
| 2705 | 15403 |
| 2706 | 15390 |
| 2707 | 14932 |
| 2708 | 14764 |
| 2709 | 15403 |
| 2710 | 4342 |
| 2711 | 14764 |
| 2712 | 14253 |
| 2713 | 15403 |
| 2714 | 4781 |
| 2715 | 13981 |
| 2716 | 4029 |
| 2717 | 3952 |
| 2718 | 14252 |
| 2719 | 14999 |
| 2720 | 4029 |
| 2721 | 4781 |
| 2722 | 3926 |
| 2723 | 4767 |
| 2724 | 4342 |
| 2725 | 4029 |
| 2726 | 14252 |
| 2727 | 3926 |
| 2728 | 3952 |
| 2729 | 3940 |
| 2730 | 4029 |
| 2731 | 15403 |
| 2732 | 14252 |
| 2733 | 4342 |
| 2734 | 14921 |
| 2735 | 13981 |
| 2736 | 15403 |
| 2737 | 15403 |
| 2738 | 3952 |
| 2739 | 4767 |
| 2740 | 4767 |
| 2741 | 14764 |
| 2742 | 3574 |
| 2743 | 15403 |
| 2744 | 14263 |
| 2745 | 14253 |
| 2746 | 4781 |
| 2747 | 4029 |
| 2748 | 14999 |
| 2749 | 14921 |
| 2750 | 4394 |
| 2751 | 14999 |
| 2752 | 14899 |
| 2753 | 14253 |
| 2754 | 4121 |
| 2755 | 14974 |
| 2756 | 3940 |
| 2757 | 4394 |
| 2758 | 4394 |
| 2759 | 4029 |
| 2760 | 14252 |
| 2761 | 14899 |
| 2762 | 4029 |
| 2763 | 4121 |
| 2764 | 15403 |
| 2765 | 14921 |
| 2766 | 14999 |
| 2767 | 4130 |
| 2768 | 4029 |
| 2769 | 13981 |
| 2770 | 14252 |
| 2771 | 14253 |
| 2772 | 14999 |
| 2773 | 4394 |
| 2774 | 14252 |
| 2775 | 14932 |
| 2776 | 14921 |
| 2777 | 15395 |
| 2778 | 14999 |
| 2779 | 14921 |
| 2780 | 14252 |
| 2781 | 14999 |
| 2782 | 4342 |
| 2783 | 4342 |
| 2784 | 4342 |
| 2785 | 14253 |
| 2786 | 4121 |
| 2787 | 4121 |
| 2788 | 4781 |
| 2789 | 14999 |
| 2790 | 14999 |
| 2791 | 4767 |
| 2792 | 4342 |
| 2793 | 14932 |
| 2794 | 3940 |
| 2795 | 4130 |
| 2796 | 14899 |
| 2797 | 13981 |
| 2798 | 4781 |
| 2799 | 4342 |
| 2800 | 15395 |
| 2801 | 4767 |
| 2802 | 14252 |
| 2803 | 4342 |
| 2804 | 4767 |
| 2805 | 4029 |
| 2806 | 3574 |
| 2807 | 14921 |
| 2808 | 14999 |
| 2809 | 4767 |
| 2810 | 14999 |
| 2811 | 4130 |
| 2812 | 8767 |
| 2813 | 13981 |
| 2814 | 14020 |
| 2815 | 15356 |
| 2816 | 14999 |
| 2817 | 15395 |
| 2818 | 14999 |
| 2819 | 4781 |
| 2820 | 14764 |
| 2821 | 14764 |
| 2822 | 14027 |
| 2823 | 14999 |
| 2824 | 4394 |
| 2825 | 4342 |
| 2826 | 4029 |
| 2827 | 14921 |
| 2828 | 4781 |
| 2829 | 15350 |
| 2830 | 14932 |
| 2831 | 15350 |
| 2832 | 14974 |
| 2833 | 14921 |
| 2834 | 4130 |
| 2835 | 4029 |
| 2836 | 14027 |
| 2837 | 14899 |
| 2838 | 15356 |
| 2839 | 14253 |
| 2840 | 13983 |
| 2841 | 14253 |
| 2842 | 14932 |
| 2843 | 14999 |
| 2844 | 4029 |
| 2845 | 15395 |
| 2846 | 13982 |
| 2847 | 13981 |
| 2848 | 4767 |
| 2849 | 15385 |
| 2850 | 4781 |
| 2851 | 4130 |
| 2852 | 14921 |
| 2853 | 14921 |
| 2854 | 13981 |
| 2855 | 4121 |
| 2856 | 4121 |
| 2857 | 14899 |
| 2858 | 14974 |
| 2859 | 14921 |
| 2860 | 14999 |
| 2861 | 14250 |
| 2862 | 14921 |
| 2863 | 4781 |
| 2864 | 4342 |
| 2865 | 15385 |
| 2866 | 14974 |
| 2867 | 13981 |
| 2868 | 14764 |
| 2869 | 4342 |
| 2870 | 4130 |
| 2871 | 15403 |
| 2872 | 4342 |
| 2873 | 14252 |
| 2874 | 14932 |
| 2875 | 4395 |
| 2876 | 14027 |
| 2877 | 14250 |
| 2878 | 15385 |
| 2879 | 15403 |
| 2880 | 14252 |
| 2881 | 14252 |
| 2882 | 14921 |
| 2883 | 4782 |
| 2884 | 5184 |
| 2885 | 14921 |
| 2886 | 8767 |
| 2887 | 4121 |
| 2888 | 14999 |
| 2889 | 15395 |
| 2890 | 15390 |
| 2891 | 3574 |
| 2892 | 14252 |
| 2893 | 14253 |
| 2894 | 14252 |
| 2895 | 4342 |
| 2896 | 4342 |
| 2897 | 15395 |
| 2898 | 4029 |
| 2899 | 15350 |
| 2900 | 14899 |
| 2901 | 14932 |
| 2902 | 14937 |
| 2903 | 4394 |
| 2904 | 4342 |
| 2905 | 4130 |
| 2906 | 14921 |
| 2907 | 14263 |
| 2908 | 14921 |
| 2909 | 3964 |
| 2910 | 4395 |
| 2911 | 3574 |
| 2912 | 14921 |
| 2913 | 4782 |
| 2914 | 15395 |
| 2915 | 4121 |
| 2916 | 4782 |
| 2917 | 14899 |
| 2918 | 14764 |
| 2919 | 14974 |
| 2920 | 4394 |
| 2921 | 4781 |
| 2922 | 3574 |
| 2923 | 15350 |
| 2924 | 4029 |
| 2925 | 4342 |
| 2926 | 15403 |
| 2927 | 4029 |
| 2928 | 14263 |
| 2929 | 4395 |
| 2930 | 15350 |
| 2931 | 3574 |
| 2932 | 14937 |
| 2933 | 14974 |
| 2934 | 14921 |
| 2935 | 3574 |
| 2936 | 14899 |
| 2937 | 14252 |
| 2938 | 14921 |
| 2939 | 13981 |
| 2940 | 14999 |
| 2941 | 14263 |
| 2942 | 14253 |
| 2943 | 14932 |
| 2944 | 14899 |
| 2945 | 3952 |
| 2946 | 13982 |
| 2947 | 15395 |
| 2948 | 15403 |
| 2949 | 3964 |
| 2950 | 3926 |
| 2951 | 4781 |
| 2952 | 14252 |
| 2953 | 3952 |
| 2954 | 3574 |
| 2955 | 14921 |
| 2956 | 14252 |
| 2957 | 14252 |
| 2958 | 4130 |
| 2959 | 15395 |
| 2960 | 14999 |
| 2961 | 4029 |
| 2962 | 4121 |
| 2963 | 15390 |
| 2964 | 13982 |
| 2965 | 3940 |
| 2966 | 4029 |
| 2967 | 14764 |
| 2968 | 14764 |
| 2969 | 4342 |
| 2970 | 14974 |
| 2971 | 14764 |
| 2972 | 14020 |
| 2973 | 15356 |
| 2974 | 14921 |
| 2975 | 15403 |
| 2976 | 4767 |
| 2977 | 14999 |
| 2978 | 4342 |
| 2979 | 15403 |
| 2980 | 4781 |
| 2981 | 14921 |
| 2982 | 14027 |
| 2983 | 15356 |
| 2984 | 15395 |
| 2985 | 3940 |
| 2986 | 14250 |
| 2987 | 15395 |
| 2988 | 14253 |
| 2989 | 14999 |
| 2990 | 14764 |
| 2991 | 3574 |
| 2992 | 14027 |
| 2993 | 3964 |
| 2994 | 14932 |
| 2995 | 14252 |
| 2996 | 14921 |
| 2997 | 14250 |
| 2998 | 15385 |
| 2999 | 4782 |
| 3000 | 14999 |
| 3001 | 4394 |
| 3002 | 14252 |
| 3003 | 3574 |
| 3004 | 4781 |
| 3005 | 15385 |
| 3006 | 14999 |
| 3007 | 14932 |
| 3008 | 14999 |
| 3009 | 4782 |
| 3010 | 15350 |
| 3011 | 15350 |
| 3012 | 4767 |
| 3013 | 15390 |
| 3014 | 3574 |
| 3015 | 15403 |
| 3016 | 14252 |
| 3017 | 4781 |
| 3018 | 14252 |
| 3019 | 3574 |
| 3020 | 14027 |
| 3021 | 4781 |
| 3022 | 13983 |
| 3023 | 15403 |
| 3024 | 4342 |
| 3025 | 14999 |
| 3026 | 15403 |
| 3027 | 4130 |
| 3028 | 14252 |
| 3029 | 4394 |
| 3030 | 4782 |
| 3031 | 4130 |
| 3032 | 14999 |
| 3033 | 4121 |
| 3034 | 14027 |
| 3035 | 14252 |
| 3036 | 4342 |
| 3037 | 14932 |
| 3038 | 14974 |
| 3039 | 15390 |
| 3040 | 14999 |
| 3041 | 5184 |
| 3042 | 14921 |
| 3043 | 15403 |
| 3044 | 4029 |
| 3045 | 15356 |
| 3046 | 14974 |
| 3047 | 14764 |
| 3048 | 4781 |
| 3049 | 14921 |
| 3050 | 4029 |
| 3051 | 13981 |
| 3052 | 14932 |
| 3053 | 14027 |
| 3054 | 4781 |
| 3055 | 4130 |
| 3056 | 14921 |
| 3057 | 3574 |
| 3058 | 3926 |
| 3059 | 14253 |
| 3060 | 13981 |
| 3061 | 4121 |
| 3062 | 4781 |
| 3063 | 14263 |
| 3064 | 15385 |
| 3065 | 13981 |
| 3066 | 15403 |
| 3067 | 15356 |
| 3068 | 14252 |
| 3069 | 14921 |
| 3070 | 8767 |
| 3071 | 14027 |
| 3072 | 15385 |
| 3073 | 15403 |
| 3074 | 14764 |
| 3075 | 14253 |
| 3076 | 4342 |
| 3077 | 14999 |
| 3078 | 4394 |
| 3079 | 15350 |
| 3080 | 14027 |
| 3081 | 15403 |
| 3082 | 4130 |
| 3083 | 3964 |
| 3084 | 4394 |
| 3085 | 14861 |
| 3086 | 4781 |
| 3087 | 3952 |
| 3088 | 13981 |
| 3089 | 3574 |
| 3090 | 4395 |
| 3091 | 14921 |
| 3092 | 14027 |
| 3093 | 3574 |
| 3094 | 4121 |
| 3095 | 14999 |
| 3096 | 15395 |
| 3097 | 15395 |
| 3098 | 14027 |
| 3099 | 4029 |
| 3100 | 15403 |
| 3101 | 14027 |
| 3102 | 4767 |
| 3103 | 15403 |
| 3104 | 4342 |
| 3105 | 15356 |
| 3106 | 14764 |
| 3107 | 3940 |
| 3108 | 14027 |
| 3109 | 15395 |
| 3110 | 3964 |
| 3111 | 14932 |
| 3112 | 4130 |
| 3113 | 14899 |
| 3114 | 4029 |
| 3115 | 3574 |
| 3116 | 14921 |
| 3117 | 15385 |
| 3118 | 15403 |
| 3119 | 14250 |
| 3120 | 14252 |
| 3121 | 3964 |
| 3122 | 14263 |
| 3123 | 14899 |
| 3124 | 13981 |
| 3125 | 15403 |
| 3126 | 14999 |
| 3127 | 4394 |
| 3128 | 14250 |
| 3129 | 14252 |
| 3130 | 3574 |
| 3131 | 4781 |
| 3132 | 15395 |
| 3133 | 14937 |
| 3134 | 14252 |
| 3135 | 4130 |
| 3136 | 14921 |
| 3137 | 3940 |
| 3138 | 4121 |
| 3139 | 14027 |
| 3140 | 14999 |
| 3141 | 4394 |
| 3142 | 14252 |
| 3143 | 3940 |
| 3144 | 15403 |
| 3145 | 14899 |
| 3146 | 14263 |
| 3147 | 14252 |
| 3148 | 14921 |
| 3149 | 14764 |
| 3150 | 14252 |
| 3151 | 4342 |
| 3152 | 3952 |
| 3153 | 15403 |
| 3154 | 14252 |
| 3155 | 13982 |
| 3156 | 3952 |
| 3157 | 3964 |
| 3158 | 3574 |
| 3159 | 14252 |
| 3160 | 3940 |
| 3161 | 15403 |
| 3162 | 4342 |
| 3163 | 3574 |
| 3164 | 15350 |
| 3165 | 4767 |
| 3166 | 14921 |
| 3167 | 4029 |
| 3168 | 4342 |
| 3169 | 14764 |
| 3170 | 14937 |
| 3171 | 14252 |
| 3172 | 4342 |
| 3173 | 14899 |
| 3174 | 15385 |
| 3175 | 14764 |
| 3176 | 4029 |
| 3177 | 15395 |
| 3178 | 4130 |
| 3179 | 14999 |
| 3180 | 14252 |
| 3181 | 14921 |
| 3182 | 14921 |
| 3183 | 4767 |
| 3184 | 3940 |
| 3185 | 4781 |
| 3186 | 4029 |
| 3187 | 4767 |
| 3188 | 15350 |
| 3189 | 14252 |
| 3190 | 4342 |
| 3191 | 14764 |
| 3192 | 14764 |
| 3193 | 8767 |
| 3194 | 3952 |
| 3195 | 3940 |
| 3196 | 15350 |
| 3197 | 14764 |
| 3198 | 14921 |
| 3199 | 3940 |
| 3200 | 14921 |
| 3201 | 14252 |
| 3202 | 14974 |
| 3203 | 14999 |
| 3204 | 14252 |
| 3205 | 14921 |
| 3206 | 15403 |
| 3207 | 14764 |
| 3208 | 4121 |
| 3209 | 14999 |
| 3210 | 14999 |
| 3211 | 3964 |
| 3212 | 15356 |
| 3213 | 4121 |
| 3214 | 4029 |
| 3215 | 14899 |
| 3216 | 14252 |
| 3217 | 4767 |
| 3218 | 14999 |
| 3219 | 13981 |
| 3220 | 14253 |
| 3221 | 3574 |
| 3222 | 15403 |
| 3223 | 3574 |
| 3224 | 4029 |
| 3225 | 3926 |
| 3226 | 3574 |
| 3227 | 4342 |
| 3228 | 4029 |
| 3229 | 14999 |
| 3230 | 14253 |
| 3231 | 4342 |
| 3232 | 14921 |
| 3233 | 14899 |
| 3234 | 14999 |
| 3235 | 3964 |
| 3236 | 14250 |
| 3237 | 14921 |
| 3238 | 3940 |
| 3239 | 4029 |
| 3240 | 13981 |
| 3241 | 4781 |
| 3242 | 4781 |
| 3243 | 14252 |
| 3244 | 14974 |
| 3245 | 4781 |
| 3246 | 14921 |
| 3247 | 4781 |
| 3248 | 14764 |
| 3249 | 3940 |
| 3250 | 14027 |
| 3251 | 14899 |
| 3252 | 14764 |
| 3253 | 4121 |
| 3254 | 15403 |
| 3255 | 14252 |
| 3256 | 4029 |
| 3257 | 5184 |
| 3258 | 14020 |
| 3259 | 14974 |
| 3260 | 14020 |
| 3261 | 14974 |
| 3262 | 4121 |
| 3263 | 14999 |
| 3264 | 14921 |
| 3265 | 15403 |
| 3266 | 14899 |
| 3267 | 4342 |
| 3268 | 14764 |
| 3269 | 14253 |
| 3270 | 14921 |
| 3271 | 3964 |
| 3272 | 15390 |
| 3273 | 14861 |
| 3274 | 14921 |
| 3275 | 14764 |
| 3276 | 14253 |
| 3277 | 3574 |
| 3278 | 3926 |
| 3279 | 3926 |
| 3280 | 14253 |
| 3281 | 15385 |
| 3282 | 4342 |
| 3283 | 14921 |
| 3284 | 14921 |
| 3285 | 14263 |
| 3286 | 4029 |
| 3287 | 3952 |
| 3288 | 14999 |
| 3289 | 4342 |
| 3290 | 15385 |
| 3291 | 4781 |
| 3292 | 14252 |
| 3293 | 4130 |
| 3294 | 4767 |
| 3295 | 14861 |
| 3296 | 14921 |
| 3297 | 3952 |
| 3298 | 14252 |
| 3299 | 14999 |
| 3300 | 4781 |
| 3301 | 3964 |
| 3302 | 4342 |
| 3303 | 13981 |
| 3304 | 14899 |
| 3305 | 4782 |
| 3306 | 15356 |
| 3307 | 15403 |
| 3308 | 14764 |
| 3309 | 4342 |
| 3310 | 14263 |
| 3311 | 14999 |
| 3312 | 14921 |
| 3313 | 14937 |
| 3314 | 14921 |
| 3315 | 8767 |
| 3316 | 14764 |
| 3317 | 3574 |
| 3318 | 4394 |
| 3319 | 13981 |
| 3320 | 14764 |
| 3321 | 4342 |
| 3322 | 14899 |
| 3323 | 14899 |
| 3324 | 14921 |
| 3325 | 14253 |
| 3326 | 14899 |
| 3327 | 13981 |
| 3328 | 15385 |
| 3329 | 4782 |
| 3330 | 4767 |
| 3331 | 14253 |
| 3332 | 13981 |
| 3333 | 15385 |
| 3334 | 14253 |
| 3335 | 4781 |
| 3336 | 14764 |
| 3337 | 14899 |
| 3338 | 15350 |
| 3339 | 14027 |
| 3340 | 4781 |
| 3341 | 4781 |
| 3342 | 4342 |
| 3343 | 14250 |
| 3344 | 3952 |
| 3345 | 4342 |
| 3346 | 14250 |
| 3347 | 14974 |
| 3348 | 4394 |
| 3349 | 3940 |
| 3350 | 14764 |
| 3351 | 3574 |
| 3352 | 14253 |
| 3353 | 14974 |
| 3354 | 14764 |
| 3355 | 15395 |
| 3356 | 5184 |
| 3357 | 4781 |
| 3358 | 3940 |
| 3359 | 4781 |
| 3360 | 4395 |
| 3361 | 14999 |
| 3362 | 4781 |
| 3363 | 14999 |
| 3364 | 14921 |
| 3365 | 3964 |
| 3366 | 4395 |
| 3367 | 14252 |
| 3368 | 3964 |
| 3369 | 4395 |
| 3370 | 14999 |
| 3371 | 15403 |
| 3372 | 14252 |
| 3373 | 14253 |
| 3374 | 14764 |
| 3375 | 4394 |
| 3376 | 14252 |
| 3377 | 14020 |
| 3378 | 4342 |
| 3379 | 4130 |
| 3380 | 3952 |
| 3381 | 3574 |
| 3382 | 14999 |
| 3383 | 4130 |
| 3384 | 4781 |
| 3385 | 14899 |
| 3386 | 15350 |
| 3387 | 14932 |
| 3388 | 14921 |
| 3389 | 14999 |
| 3390 | 14252 |
| 3391 | 4781 |
| 3392 | 4342 |
| 3393 | 14252 |
| 3394 | 14999 |
| 3395 | 4781 |
| 3396 | 4394 |
| 3397 | 4342 |
| 3398 | 14764 |
| 3399 | 3926 |
| 3400 | 15403 |
| 3401 | 14252 |
| 3402 | 3952 |
| 3403 | 14932 |
| 3404 | 4029 |
| 3405 | 3964 |
| 3406 | 4767 |
| 3407 | 14252 |
| 3408 | 3964 |
| 3409 | 14974 |
| 3410 | 15350 |
| 3411 | 3574 |
| 3412 | 14899 |
| 3413 | 4342 |
| 3414 | 4029 |
| 3415 | 3952 |
| 3416 | 14921 |
| 3417 | 13981 |
| 3418 | 4029 |
| 3419 | 4029 |
| 3420 | 4782 |
| 3421 | 15385 |
| 3422 | 4029 |
| 3423 | 15350 |
| 3424 | 14027 |
| 3425 | 14263 |
| 3426 | 13981 |
| 3427 | 4342 |
| 3428 | 13981 |
| 3429 | 14921 |
| 3430 | 14899 |
| 3431 | 13981 |
| 3432 | 14932 |
| 3433 | 14932 |
| 3434 | 14921 |
| 3435 | 4029 |
| 3436 | 4130 |
| 3437 | 14999 |
| 3438 | 14937 |
| 3439 | 4342 |
| 3440 | 15350 |
| 3441 | 4130 |
| 3442 | 4395 |
| 3443 | 13983 |
| 3444 | 14027 |
| 3445 | 14999 |
| 3446 | 4342 |
| 3447 | 4029 |
| 3448 | 14252 |
| 3449 | 4781 |
| 3450 | 13982 |
| 3451 | 14263 |
| 3452 | 15403 |
| 3453 | 3952 |
| 3454 | 14764 |
| 3455 | 14252 |
| 3456 | 14764 |
| 3457 | 3964 |
| 3458 | 14921 |
| 3459 | 15403 |
| 3460 | 14252 |
| 3461 | 15350 |
| 3462 | 4029 |
| 3463 | 14252 |
| 3464 | 4781 |
| 3465 | 4029 |
| 3466 | 13982 |
| 3467 | 13982 |
| 3468 | 14974 |
| 3469 | 4781 |
| 3470 | 14999 |
| 3471 | 4130 |
| 3472 | 15403 |
| 3473 | 14027 |
| 3474 | 14999 |
| 3475 | 4781 |
| 3476 | 14252 |
| 3477 | 4782 |
| 3478 | 15385 |
| 3479 | 15395 |
| 3480 | 14764 |
| 3481 | 4121 |
| 3482 | 14921 |
| 3483 | 3574 |
| 3484 | 13981 |
| 3485 | 15356 |
| 3486 | 14764 |
| 3487 | 15390 |
| 3488 | 14999 |
| 3489 | 15395 |
| 3490 | 4342 |
| 3491 | 14921 |
| 3492 | 14764 |
| 3493 | 4130 |
| 3494 | 14253 |
| 3495 | 14252 |
| 3496 | 15385 |
| 3497 | 14999 |
| 3498 | 4781 |
| 3499 | 15385 |
| 3500 | 14252 |
| 3501 | 13982 |
| 3502 | 4029 |
| 3503 | 3574 |
| 3504 | 14764 |
| 3505 | 14252 |
| 3506 | 14250 |
| 3507 | 4121 |
| 3508 | 4781 |
| 3509 | 3964 |
| 3510 | 14253 |
| 3511 | 4121 |
| 3512 | 15403 |
| 3513 | 3940 |
| 3514 | 14252 |
| 3515 | 14252 |
| 3516 | 14999 |
| 3517 | 4394 |
| 3518 | 13983 |
| 3519 | 14252 |
| 3520 | 14764 |
| 3521 | 8767 |
| 3522 | 14252 |
| 3523 | 3574 |
| 3524 | 13981 |
| 3525 | 14253 |
| 3526 | 14999 |
| 3527 | 14999 |
| 3528 | 14764 |
| 3529 | 13981 |
| 3530 | 15395 |
| 3531 | 4782 |
| 3532 | 14027 |
| 3533 | 4342 |
| 3534 | 4781 |
| 3535 | 14974 |
| 3536 | 4342 |
| 3537 | 4781 |
| 3538 | 3940 |
| 3539 | 14999 |
| 3540 | 5184 |
| 3541 | 14999 |
| 3542 | 4781 |
| 3543 | 14027 |
| 3544 | 14252 |
| 3545 | 4394 |
| 3546 | 4029 |
| 3547 | 14974 |
| 3548 | 15403 |
| 3549 | 14252 |
| 3550 | 14252 |
| 3551 | 4767 |
| 3552 | 4767 |
| 3553 | 8767 |
| 3554 | 14999 |
| 3555 | 4781 |
| 3556 | 4121 |
| 3557 | 4767 |
| 3558 | 14999 |
| 3559 | 8767 |
| 3560 | 15390 |
| 3561 | 3926 |
| 3562 | 14263 |
| 3563 | 3574 |
| 3564 | 14027 |
| 3565 | 14999 |
| 3566 | 14999 |
| 3567 | 4121 |
| 3568 | 4121 |
| 3569 | 4781 |
| 3570 | 15350 |
| 3571 | 4767 |
| 3572 | 4395 |
| 3573 | 3940 |
| 3574 | 14263 |
| 3575 | 14899 |
| 3576 | 14764 |
| 3577 | 14764 |
| 3578 | 4781 |
| 3579 | 4782 |
| 3580 | 3964 |
| 3581 | 14027 |
| 3582 | 3964 |
| 3583 | 14932 |
| 3584 | 14921 |
| 3585 | 14020 |
| 3586 | 14921 |
| 3587 | 14250 |
| 3588 | 14899 |
| 3589 | 4781 |
| 3590 | 14921 |
| 3591 | 14937 |
| 3592 | 3940 |
| 3593 | 15385 |
| 3594 | 14253 |
| 3595 | 3926 |
| 3596 | 14027 |
| 3597 | 3574 |
| 3598 | 15403 |
| 3599 | 14253 |
| 3600 | 14253 |
| 3601 | 8767 |
| 3602 | 14899 |
| 3603 | 4342 |
| 3604 | 4029 |
| 3605 | 14764 |
| 3606 | 15403 |
| 3607 | 13981 |
| 3608 | 14252 |
| 3609 | 4781 |
| 3610 | 4781 |
| 3611 | 15403 |
| 3612 | 3964 |
| 3613 | 14861 |
| 3614 | 14937 |
| 3615 | 14027 |
| 3616 | 4781 |
| 3617 | 14764 |
| 3618 | 15385 |
| 3619 | 15385 |
| 3620 | 14252 |
| 3621 | 14253 |
| 3622 | 14027 |
| 3623 | 3952 |
| 3624 | 4767 |
| 3625 | 14999 |
| 3626 | 5184 |
| 3627 | 4767 |
| 3628 | 14974 |
| 3629 | 14027 |
| 3630 | 14764 |
| 3631 | 14764 |
| 3632 | 14974 |
| 3633 | 5184 |
| 3634 | 3964 |
| 3635 | 15356 |
| 3636 | 4767 |
| 3637 | 14263 |
| 3638 | 15395 |
| 3639 | 4781 |
| 3640 | 14027 |
| 3641 | 14764 |
| 3642 | 4029 |
| 3643 | 3940 |
| 3644 | 4342 |
| 3645 | 4767 |
| 3646 | 3574 |
| 3647 | 4121 |
| 3648 | 4781 |
| 3649 | 4342 |
| 3650 | 14999 |
| 3651 | 14020 |
| 3652 | 14937 |
| 3653 | 4029 |
| 3654 | 4781 |
| 3655 | 3964 |
| 3656 | 13981 |
| 3657 | 14999 |
| 3658 | 14764 |
| 3659 | 14899 |
| 3660 | 4029 |
| 3661 | 14252 |
| 3662 | 4342 |
| 3663 | 14027 |
| 3664 | 15356 |
| 3665 | 4781 |
| 3666 | 14861 |
| 3667 | 14253 |
| 3668 | 15390 |
| 3669 | 4029 |
| 3670 | 13981 |
| 3671 | 14253 |
| 3672 | 14937 |
| 3673 | 13981 |
| 3674 | 14999 |
| 3675 | 15385 |
| 3676 | 14252 |
| 3677 | 3940 |
| 3678 | 4394 |
| 3679 | 4342 |
| 3680 | 4342 |
| 3681 | 14263 |
| 3682 | 14974 |
| 3683 | 13981 |
| 3684 | 13983 |
| 3685 | 15350 |
| 3686 | 4342 |
| 3687 | 14899 |
| 3688 | 4130 |
| 3689 | 4342 |
| 3690 | 14937 |
| 3691 | 14999 |
| 3692 | 15350 |
| 3693 | 3940 |
| 3694 | 4767 |
| 3695 | 14921 |
| 3696 | 3940 |
| 3697 | 4394 |
| 3698 | 13983 |
| 3699 | 4029 |
| 3700 | 4130 |
| 3701 | 14999 |
| 3702 | 3926 |
| 3703 | 4767 |
| 3704 | 14027 |
| 3705 | 13981 |
| 3706 | 4029 |
| 3707 | 3940 |
| 3708 | 14252 |
| 3709 | 14764 |
| 3710 | 3574 |
| 3711 | 3940 |
| 3712 | 4342 |
| 3713 | 4767 |
| 3714 | 14764 |
| 3715 | 4029 |
| 3716 | 14764 |
| 3717 | 14764 |
| 3718 | 3574 |
| 3719 | 14252 |
| 3720 | 15390 |
| 3721 | 4781 |
| 3722 | 15403 |
| 3723 | 13981 |
| 3724 | 4781 |
| 3725 | 5184 |
| 3726 | 15385 |
| 3727 | 15403 |
| 3728 | 4029 |
| 3729 | 4767 |
| 3730 | 14764 |
| 3731 | 14937 |
| 3732 | 14932 |
| 3733 | 4781 |
| 3734 | 14252 |
| 3735 | 14899 |
| 3736 | 4130 |
| 3737 | 4781 |
| 3738 | 14899 |
| 3739 | 4767 |
| 3740 | 4342 |
| 3741 | 14027 |
| 3742 | 4029 |
| 3743 | 14764 |
| 3744 | 14250 |
| 3745 | 14899 |
| 3746 | 14250 |
| 3747 | 14999 |
| 3748 | 4767 |
| 3749 | 3964 |
| 3750 | 4121 |
| 3751 | 15390 |
| 3752 | 14764 |
| 3753 | 3926 |
| 3754 | 14027 |
| 3755 | 8767 |
| 3756 | 4130 |
| 3757 | 15395 |
| 3758 | 14252 |
| 3759 | 4781 |
| 3760 | 14921 |
| 3761 | 15385 |
| 3762 | 3964 |
| 3763 | 4781 |
| 3764 | 14764 |
| 3765 | 13981 |
| 3766 | 4767 |
| 3767 | 14921 |
| 3768 | 14899 |
| 3769 | 4781 |
| 3770 | 15356 |
| 3771 | 14937 |
| 3772 | 3574 |
| 3773 | 15390 |
| 3774 | 14999 |
| 3775 | 14252 |
| 3776 | 4394 |
| 3777 | 15403 |
| 3778 | 13981 |
| 3779 | 14974 |
| 3780 | 15356 |
| 3781 | 13981 |
| 3782 | 14999 |
| 3783 | 14252 |
| 3784 | 4029 |
| 3785 | 15403 |
| 3786 | 13981 |
| 3787 | 3940 |
| 3788 | 14252 |
| 3789 | 14764 |
| 3790 | 14932 |
| 3791 | 14252 |
| 3792 | 15403 |
| 3793 | 13981 |
| 3794 | 4342 |
| 3795 | 4130 |
| 3796 | 14252 |
| 3797 | 5184 |
| 3798 | 13981 |
| 3799 | 4121 |
| 3800 | 3952 |
| 3801 | 3574 |
| 3802 | 15350 |
| 3803 | 3574 |
| 3804 | 14999 |
| 3805 | 14253 |
| 3806 | 14027 |
| 3807 | 3952 |
| 3808 | 15385 |
| 3809 | 4342 |
| 3810 | 4342 |
| 3811 | 4029 |
| 3812 | 14899 |
| 3813 | 14921 |
| 3814 | 14932 |
| 3815 | 4767 |
| 3816 | 4781 |
| 3817 | 15350 |
| 3818 | 3964 |
| 3819 | 15403 |
| 3820 | 4029 |
| 3821 | 14999 |
| 3822 | 4781 |
| 3823 | 14899 |
| 3824 | 14027 |
| 3825 | 14937 |
| 3826 | 14764 |
| 3827 | 4767 |
| 3828 | 14253 |
| 3829 | 4781 |
| 3830 | 14252 |
| 3831 | 4781 |
| 3832 | 14253 |
| 3833 | 14027 |
| 3834 | 4121 |
| 3835 | 3574 |
| 3836 | 15395 |
| 3837 | 14252 |
| 3838 | 14921 |
| 3839 | 14250 |
| 3840 | 14027 |
| 3841 | 14250 |
| 3842 | 4781 |
| 3843 | 15350 |
| 3844 | 4121 |
| 3845 | 3964 |
| 3846 | 14861 |
| 3847 | 15356 |
| 3848 | 3574 |
| 3849 | 14252 |
| 3850 | 3964 |
| 3851 | 14253 |
| 3852 | 3940 |
| 3853 | 14252 |
| 3854 | 13981 |
| 3855 | 14764 |
| 3856 | 14974 |
| 3857 | 14253 |
| 3858 | 4121 |
| 3859 | 15403 |
| 3860 | 4130 |
| 3861 | 15385 |
| 3862 | 14764 |
| 3863 | 3952 |
| 3864 | 4767 |
| 3865 | 14764 |
| 3866 | 14253 |
| 3867 | 14932 |
| 3868 | 3574 |
| 3869 | 14861 |
| 3870 | 4029 |
| 3871 | 3964 |
| 3872 | 14252 |
| 3873 | 4130 |
| 3874 | 4029 |
| 3875 | 4342 |
| 3876 | 14932 |
| 3877 | 14764 |
| 3878 | 4781 |
| 3879 | 13981 |
| 3880 | 4130 |
| 3881 | 3926 |
| 3882 | 15350 |
| 3883 | 4394 |
| 3884 | 4130 |
| 3885 | 13981 |
| 3886 | 15390 |
| 3887 | 4029 |
| 3888 | 14250 |
| 3889 | 4029 |
| 3890 | 3952 |
| 3891 | 4394 |
| 3892 | 4782 |
| 3893 | 4029 |
| 3894 | 14932 |
| 3895 | 14899 |
| 3896 | 14932 |
| 3897 | 4767 |
| 3898 | 14921 |
| 3899 | 14999 |
| 3900 | 4781 |
| 3901 | 4029 |
| 3902 | 14937 |
| 3903 | 14250 |
| 3904 | 3964 |
| 3905 | 14764 |
| 3906 | 4029 |
| 3907 | 3926 |
| 3908 | 15350 |
| 3909 | 14252 |
| 3910 | 15385 |
| 3911 | 15403 |
| 3912 | 13981 |
| 3913 | 14252 |
| 3914 | 14999 |
| 3915 | 4342 |
| 3916 | 14921 |
| 3917 | 14999 |
| 3918 | 4121 |
| 3919 | 14974 |
| 3920 | 14974 |
| 3921 | 14250 |
| 3922 | 14252 |
| 3923 | 14861 |
| 3924 | 15403 |
| 3925 | 14253 |
| 3926 | 14764 |
| 3927 | 14253 |
| 3928 | 15350 |
| 3929 | 4029 |
| 3930 | 13981 |
| 3931 | 15356 |
| 3932 | 4130 |
| 3933 | 3940 |
| 3934 | 4781 |
| 3935 | 13981 |
| 3936 | 14252 |
| 3937 | 13981 |
| 3938 | 14999 |
| 3939 | 4781 |
| 3940 | 14999 |
| 3941 | 14999 |
| 3942 | 14252 |
| 3943 | 4130 |
| 3944 | 15356 |
| 3945 | 4767 |
| 3946 | 14974 |
| 3947 | 14999 |
| 3948 | 14937 |
| 3949 | 4782 |
| 3950 | 4781 |
| 3951 | 15385 |
| 3952 | 14899 |
| 3953 | 13981 |
| 3954 | 4781 |
| 3955 | 14999 |
| 3956 | 4029 |
| 3957 | 4121 |
| 3958 | 4342 |
| 3959 | 14899 |
| 3960 | 14253 |
| 3961 | 4781 |
| 3962 | 14974 |
| 3963 | 14999 |
| 3964 | 4781 |
| 3965 | 14252 |
| 3966 | 14999 |
| 3967 | 4767 |
| 3968 | 14937 |
| 3969 | 15390 |
| 3970 | 14921 |
| 3971 | 3940 |
| 3972 | 14932 |
| 3973 | 14974 |
| 3974 | 14999 |
| 3975 | 4121 |
| 3976 | 14932 |
| 3977 | 4394 |
| 3978 | 3964 |
| 3979 | 14252 |
| 3980 | 14027 |
| 3981 | 4029 |
| 3982 | 4781 |
| 3983 | 4395 |
| 3984 | 15350 |
| 3985 | 14764 |
| 3986 | 14932 |
| 3987 | 14999 |
| 3988 | 3940 |
| 3989 | 14999 |
| 3990 | 14252 |
| 3991 | 14764 |
| 3992 | 8767 |
| 3993 | 13981 |
| 3994 | 14999 |
| 3995 | 14252 |
| 3996 | 14899 |
| 3997 | 14921 |
| 3998 | 4394 |
| 3999 | 14999 |
| 4000 | 14253 |
| 4001 | 3964 |
| 4002 | 14250 |
| 4003 | 15390 |
| 4004 | 4781 |
| 4005 | 4029 |
| 4006 | 14999 |
| 4007 | 4782 |
| 4008 | 14020 |
| 4009 | 14999 |
| 4010 | 4130 |
| 4011 | 4029 |
| 4012 | 3964 |
| 4013 | 3940 |
| 4014 | 15350 |
| 4015 | 14764 |
| 4016 | 4342 |
| 4017 | 4395 |
| 4018 | 13981 |
| 4019 | 14764 |
| 4020 | 15350 |
| 4021 | 3940 |
| 4022 | 14764 |
| 4023 | 3926 |
| 4024 | 14250 |
| 4025 | 15390 |
| 4026 | 15403 |
| 4027 | 13981 |
| 4028 | 4782 |
| 4029 | 4029 |
| 4030 | 14921 |
| 4031 | 14253 |
| 4032 | 14899 |
| 4033 | 13981 |
| 4034 | 14921 |
| 4035 | 4394 |
| 4036 | 14020 |
| 4037 | 14253 |
| 4038 | 14999 |
| 4039 | 4395 |
| 4040 | 3964 |
| 4041 | 8767 |
| 4042 | 14250 |
| 4043 | 15403 |
| 4044 | 14861 |
| 4045 | 4395 |
| 4046 | 14932 |
| 4047 | 3574 |
| 4048 | 14974 |
| 4049 | 15395 |
| 4050 | 3574 |
| 4051 | 4029 |
| 4052 | 14027 |
| 4053 | 4342 |
| 4054 | 4781 |
| 4055 | 14764 |
| 4056 | 14921 |
| 4057 | 15403 |
| 4058 | 4781 |
| 4059 | 4782 |
| 4060 | 14250 |
| 4061 | 4342 |
| 4062 | 3574 |
| 4063 | 4767 |
| 4064 | 15403 |
| 4065 | 3574 |
| 4066 | 15350 |
| 4067 | 3926 |
| 4068 | 14999 |
| 4069 | 15403 |
| 4070 | 14999 |
| 4071 | 14999 |
| 4072 | 15350 |
| 4073 | 4394 |
| 4074 | 15403 |
| 4075 | 4029 |
| 4076 | 4029 |
| 4077 | 14974 |
| 4078 | 14252 |
| 4079 | 14020 |
| 4080 | 14937 |
| 4081 | 15395 |
| 4082 | 14974 |
| 4083 | 14921 |
| 4084 | 4130 |
| 4085 | 15403 |
| 4086 | 4029 |
| 4087 | 4342 |
| 4088 | 3940 |
| 4089 | 14921 |
| 4090 | 14250 |
| 4091 | 14974 |
| 4092 | 4029 |
| 4093 | 14252 |
| 4094 | 14764 |
| 4095 | 4767 |
| 4096 | 15403 |
| 4097 | 4781 |
| 4098 | 4342 |
| 4099 | 3964 |
| 4100 | 15390 |
| 4101 | 14999 |
| 4102 | 4394 |
| 4103 | 14999 |
| 4104 | 13983 |
| 4105 | 4394 |
| 4106 | 14764 |
| 4107 | 14899 |
| 4108 | 14999 |
| 4109 | 14999 |
| 4110 | 4130 |
| 4111 | 14252 |
| 4112 | 13981 |
| 4113 | 4121 |
| 4114 | 15385 |
| 4115 | 15395 |
| 4116 | 14250 |
| 4117 | 3940 |
| 4118 | 14252 |
| 4119 | 4029 |
| 4120 | 14921 |
| 4121 | 14932 |
| 4122 | 15350 |
| 4123 | 4130 |
| 4124 | 14764 |
| 4125 | 14999 |
| 4126 | 3574 |
| 4127 | 15350 |
| 4128 | 4767 |
| 4129 | 14027 |
| 4130 | 14932 |
| 4131 | 13981 |
| 4132 | 14253 |
| 4133 | 14937 |
| 4134 | 14899 |
| 4135 | 14899 |
| 4136 | 13981 |
| 4137 | 14999 |
| 4138 | 4342 |
| 4139 | 14253 |
| 4140 | 4781 |
| 4141 | 14764 |
| 4142 | 14999 |
| 4143 | 15350 |
| 4144 | 14027 |
| 4145 | 8767 |
| 4146 | 14027 |
| 4147 | 3964 |
| 4148 | 3574 |
| 4149 | 14252 |
| 4150 | 3964 |
| 4151 | 4342 |
| 4152 | 4029 |
| 4153 | 15350 |
| 4154 | 3574 |
| 4155 | 14974 |
| 4156 | 4781 |
| 4157 | 14764 |
| 4158 | 4342 |
| 4159 | 14253 |
| 4160 | 4782 |
| 4161 | 14764 |
| 4162 | 13981 |
| 4163 | 4342 |
| 4164 | 15350 |
| 4165 | 15403 |
| 4166 | 14999 |
| 4167 | 14764 |
| 4168 | 3940 |
| 4169 | 14253 |
| 4170 | 13981 |
| 4171 | 13983 |
| 4172 | 4029 |
| 4173 | 4029 |
| 4174 | 14921 |
| 4175 | 4029 |
| 4176 | 4767 |
| 4177 | 14253 |
| 4178 | 14764 |
| 4179 | 14252 |
| 4180 | 13982 |
| 4181 | 4767 |
| 4182 | 4342 |
| 4183 | 14999 |
| 4184 | 4781 |
| 4185 | 14027 |
| 4186 | 15385 |
| 4187 | 4395 |
| 4188 | 4029 |
| 4189 | 15356 |
| 4190 | 14921 |
| 4191 | 13983 |
| 4192 | 14252 |
| 4193 | 3940 |
| 4194 | 4121 |
| 4195 | 14899 |
| 4196 | 4121 |
| 4197 | 4342 |
| 4198 | 4121 |
| 4199 | 15350 |
| 4200 | 14250 |
| 4201 | 14764 |
| 4202 | 4029 |
| 4203 | 14253 |
| 4204 | 14999 |
| 4205 | 3952 |
| 4206 | 4029 |
| 4207 | 14921 |
| 4208 | 14764 |
| 4209 | 4029 |
| 4210 | 4029 |
| 4211 | 14250 |
| 4212 | 15403 |
| 4213 | 13983 |
| 4214 | 14999 |
| 4215 | 4782 |
| 4216 | 14252 |
| 4217 | 14764 |
| 4218 | 15385 |
| 4219 | 14937 |
| 4220 | 14974 |
| 4221 | 14999 |
| 4222 | 14764 |
| 4223 | 3952 |
| 4224 | 3964 |
| 4225 | 4781 |
| 4226 | 14921 |
| 4227 | 3940 |
| 4228 | 14764 |
| 4229 | 14027 |
| 4230 | 4781 |
| 4231 | 14937 |
| 4232 | 14764 |
| 4233 | 14027 |
| 4234 | 3926 |
| 4235 | 14921 |
| 4236 | 4781 |
| 4237 | 14921 |
| 4238 | 4781 |
| 4239 | 14999 |
| 4240 | 3952 |
| 4241 | 13981 |
| 4242 | 14932 |
| 4243 | 4394 |
| 4244 | 14027 |
| 4245 | 4781 |
| 4246 | 15395 |
| 4247 | 14921 |
| 4248 | 14252 |
| 4249 | 14764 |
| 4250 | 3574 |
| 4251 | 14027 |
| 4252 | 14764 |
| 4253 | 14253 |
| 4254 | 14899 |
| 4255 | 14263 |
| 4256 | 14932 |
| 4257 | 3952 |
| 4258 | 14899 |
| 4259 | 14263 |
| 4260 | 14921 |
| 4261 | 4781 |
| 4262 | 4121 |
| 4263 | 13981 |
| 4264 | 14999 |
| 4265 | 14999 |
| 4266 | 4781 |
| 4267 | 3926 |
| 4268 | 15403 |
| 4269 | 14253 |
| 4270 | 13981 |
| 4271 | 3940 |
| 4272 | 14252 |
| 4273 | 14999 |
| 4274 | 3574 |
| 4275 | 14252 |
| 4276 | 15385 |
| 4277 | 4029 |
| 4278 | 4029 |
| 4279 | 15395 |
| 4280 | 15350 |
| 4281 | 14764 |
| 4282 | 13981 |
| 4283 | 14253 |
| 4284 | 4342 |
| 4285 | 14921 |
| 4286 | 4029 |
| 4287 | 4121 |
| 4288 | 3574 |
| 4289 | 4782 |
| 4290 | 14764 |
| 4291 | 14027 |
| 4292 | 4394 |
| 4293 | 14899 |
| 4294 | 4394 |
| 4295 | 15385 |
| 4296 | 3952 |
| 4297 | 4767 |
| 4298 | 4342 |
| 4299 | 14921 |
| 4300 | 15385 |
| 4301 | 4342 |
| 4302 | 14921 |
| 4303 | 3574 |
| 4304 | 4394 |
| 4305 | 15403 |
| 4306 | 14020 |
| 4307 | 14250 |
| 4308 | 4342 |
| 4309 | 4029 |
| 4310 | 14921 |
| 4311 | 4029 |
| 4312 | 15403 |
| 4313 | 14253 |
| 4314 | 14921 |
| 4315 | 4029 |
| 4316 | 8767 |
| 4317 | 4394 |
| 4318 | 14252 |
| 4319 | 14899 |
| 4320 | 14764 |
| 4321 | 14899 |
| 4322 | 14999 |
| 4323 | 4781 |
| 4324 | 14999 |
| 4325 | 14999 |
| 4326 | 4342 |
| 4327 | 4029 |
| 4328 | 15395 |
| 4329 | 4781 |
| 4330 | 13982 |
| 4331 | 4130 |
| 4332 | 4121 |
| 4333 | 14921 |
| 4334 | 14999 |
| 4335 | 14250 |
| 4336 | 14250 |
| 4337 | 3952 |
| 4338 | 3952 |
| 4339 | 4130 |
| 4340 | 13983 |
| 4341 | 3574 |
| 4342 | 14999 |
| 4343 | 15350 |
| 4344 | 14252 |
| 4345 | 5184 |
| 4346 | 14999 |
| 4347 | 14899 |
| 4348 | 4130 |
| 4349 | 13981 |
| 4350 | 14937 |
| 4351 | 3574 |
| 4352 | 15403 |
| 4353 | 14764 |
| 4354 | 14252 |
| 4355 | 14974 |
| 4356 | 4394 |
| 4357 | 4342 |
| 4358 | 4029 |
| 4359 | 14921 |
| 4360 | 4121 |
| 4361 | 15403 |
| 4362 | 14932 |
| 4363 | 4782 |
| 4364 | 15390 |
| 4365 | 4767 |
| 4366 | 4767 |
| 4367 | 14764 |
| 4368 | 14974 |
| 4369 | 14899 |
| 4370 | 14974 |
| 4371 | 4029 |
| 4372 | 3926 |
| 4373 | 14921 |
| 4374 | 4342 |
| 4375 | 3964 |
| 4376 | 15403 |
| 4377 | 3574 |
| 4378 | 15390 |
| 4379 | 3574 |
| 4380 | 4029 |
| 4381 | 4767 |
| 4382 | 15385 |
| 4383 | 4782 |
| 4384 | 14999 |
| 4385 | 14253 |
| 4386 | 4029 |
| 4387 | 3952 |
| 4388 | 4121 |
| 4389 | 4767 |
| 4390 | 15385 |
| 4391 | 15403 |
| 4392 | 14974 |
| 4393 | 14764 |
| 4394 | 4782 |
| 4395 | 13983 |
| 4396 | 14937 |
| 4397 | 4342 |
| 4398 | 14899 |
| 4399 | 4029 |
| 4400 | 14921 |
| 4401 | 3574 |
| 4402 | 14252 |
| 4403 | 13982 |
| 4404 | 3964 |
| 4405 | 15385 |
| 4406 | 14250 |
| 4407 | 14937 |
| 4408 | 4781 |
| 4409 | 14764 |
| 4410 | 4029 |
| 4411 | 14252 |
| 4412 | 4767 |
| 4413 | 15395 |
| 4414 | 14921 |
| 4415 | 14974 |
| 4416 | 14252 |
| 4417 | 13981 |
| 4418 | 14252 |
| 4419 | 4342 |
| 4420 | 14764 |
| 4421 | 14921 |
| 4422 | 14250 |
| 4423 | 14932 |
| 4424 | 15403 |
| 4425 | 14252 |
| 4426 | 14764 |
| 4427 | 3574 |
| 4428 | 4781 |
| 4429 | 3940 |
| 4430 | 15350 |
| 4431 | 4395 |
| 4432 | 4029 |
| 4433 | 15390 |
| 4434 | 4130 |
| 4435 | 4029 |
| 4436 | 13981 |
| 4437 | 3574 |
| 4438 | 4342 |
| 4439 | 15403 |
| 4440 | 14253 |
| 4441 | 14027 |
| 4442 | 14937 |
| 4443 | 15403 |
| 4444 | 13981 |
| 4445 | 4394 |
| 4446 | 4782 |
| 4447 | 14263 |
| 4448 | 3964 |
| 4449 | 4029 |
| 4450 | 13981 |
| 4451 | 3964 |
| 4452 | 3574 |
| 4453 | 14764 |
| 4454 | 14252 |
| 4455 | 4781 |
| 4456 | 4781 |
| 4457 | 14764 |
| 4458 | 4395 |
| 4459 | 14999 |
| 4460 | 14999 |
| 4461 | 14999 |
| 4462 | 15385 |
| 4463 | 4395 |
| 4464 | 14999 |
| 4465 | 4029 |
| 4466 | 14899 |
| 4467 | 15403 |
| 4468 | 14899 |
| 4469 | 4394 |
| 4470 | 4342 |
| 4471 | 14921 |
| 4472 | 14974 |
| 4473 | 4781 |
| 4474 | 14999 |
| 4475 | 14999 |
| 4476 | 14764 |
| 4477 | 4342 |
| 4478 | 14921 |
| 4479 | 14999 |
| 4480 | 4394 |
| 4481 | 13983 |
| 4482 | 15350 |
| 4483 | 3952 |
| 4484 | 4130 |
| 4485 | 4781 |
| 4486 | 15403 |
| 4487 | 14252 |
| 4488 | 4130 |
| 4489 | 14999 |
| 4490 | 4029 |
| 4491 | 14921 |
| 4492 | 14999 |
| 4493 | 14999 |
| 4494 | 14974 |
| 4495 | 15403 |
| 4496 | 13981 |
| 4497 | 15403 |
| 4498 | 14921 |
| 4499 | 14263 |
| 4500 | 14253 |
| 4501 | 14974 |
| 4502 | 15356 |
| 4503 | 4029 |
| 4504 | 14999 |
| 4505 | 14932 |
| 4506 | 15385 |
| 4507 | 15403 |
| 4508 | 14861 |
| 4509 | 14252 |
| 4510 | 14999 |
| 4511 | 4029 |
| 4512 | 4342 |
| 4513 | 13981 |
| 4514 | 14999 |
| 4515 | 15350 |
| 4516 | 15403 |
| 4517 | 14974 |
| 4518 | 14027 |
| 4519 | 4767 |
| 4520 | 14899 |
| 4521 | 14921 |
| 4522 | 4342 |
| 4523 | 15390 |
| 4524 | 14999 |
| 4525 | 14932 |
| 4526 | 14252 |
| 4527 | 14764 |
| 4528 | 4342 |
| 4529 | 15390 |
| 4530 | 14921 |
| 4531 | 4029 |
| 4532 | 13981 |
| 4533 | 4029 |
| 4534 | 14921 |
| 4535 | 14252 |
| 4536 | 4394 |
| 4537 | 4130 |
| 4538 | 14999 |
| 4539 | 14921 |
| 4540 | 15385 |
| 4541 | 14921 |
| 4542 | 14974 |
| 4543 | 3926 |
| 4544 | 3574 |
| 4545 | 4767 |
| 4546 | 15356 |
| 4547 | 15403 |
| 4548 | 14899 |
| 4549 | 14764 |
| 4550 | 14999 |
| 4551 | 14921 |
| 4552 | 14252 |
| 4553 | 15356 |
| 4554 | 14899 |
| 4555 | 14861 |
| 4556 | 15350 |
| 4557 | 14899 |
| 4558 | 15403 |
| 4559 | 3940 |
| 4560 | 4781 |
| 4561 | 4121 |
| 4562 | 3574 |
| 4563 | 15403 |
| 4564 | 14932 |
| 4565 | 4342 |
| 4566 | 15350 |
| 4567 | 3964 |
| 4568 | 14253 |
| 4569 | 3574 |
| 4570 | 14250 |
| 4571 | 14020 |
| 4572 | 13981 |
| 4573 | 4394 |
| 4574 | 15390 |
| 4575 | 3574 |
| 4576 | 8767 |
| 4577 | 4781 |
| 4578 | 14999 |
| 4579 | 14253 |
| 4580 | 4029 |
| 4581 | 15403 |
| 4582 | 14764 |
| 4583 | 14999 |
| 4584 | 14027 |
| 4585 | 14252 |
| 4586 | 4394 |
| 4587 | 14252 |
| 4588 | 14999 |
| 4589 | 4767 |
| 4590 | 14253 |
| 4591 | 14921 |
| 4592 | 14253 |
| 4593 | 14764 |
| 4594 | 14974 |
| 4595 | 14252 |
| 4596 | 4342 |
| 4597 | 14764 |
| 4598 | 14764 |
| 4599 | 14974 |
| 4600 | 4029 |
| 4601 | 14921 |
| 4602 | 14999 |
| 4603 | 13981 |
| 4604 | 14921 |
| 4605 | 15356 |
| 4606 | 4121 |
| 4607 | 15390 |
| 4608 | 14921 |
| 4609 | 14253 |
| 4610 | 4342 |
| 4611 | 15390 |
| 4612 | 3964 |
| 4613 | 14253 |
| 4614 | 14899 |
| 4615 | 4029 |
| 4616 | 4781 |
| 4617 | 15350 |
| 4618 | 13981 |
| 4619 | 3574 |
| 4620 | 15385 |
| 4621 | 3964 |
| 4622 | 14932 |
| 4623 | 14932 |
| 4624 | 14974 |
| 4625 | 3964 |
| 4626 | 4342 |
| 4627 | 14921 |
| 4628 | 3964 |

Although the invention has been described in conjunction with specific embodiments thereof, it is evident that many alternatives, modifications and variations will be apparent to those skilled in the art. Accordingly, it is intended to embrace all such alternatives, modifications and variations that fall within the spirit and broad scope of the appended claims.

All publications, patents and patent applications mentioned in this specification are herein incorporated in their entirety by reference into the specification, to the same extent as if each individual publication, patent or patent application was specifically and individually indicated to be incorporated herein by reference. In addition, citation or identification of any reference in this application shall not be construed as an admission that such reference is available as prior art to the present invention. To the extent that section headings are used, they should not be construed as necessarily limiting.

In addition, any priority document(s) of this application is/are hereby incorporated herein by reference in its/their entirety.

### REFERENCES

1. Psychogios, N. et al. The human serum metabolome. PLoS ONE 6, e16957 (2011).
2. Ridker, P. M., Stampfer, M. J. & Rifai, N. Novel risk factors for systemic atherosclerosis. JAMA 285, 2481 (2001).
3. Baigent, C. et al. Efficacy and safety of cholesterol-lowering treatment: prospective meta-analysis of data from 90,056 participants in 14 randomised trials of statins. Lancet 366, 1267-1278 (2005).
4. National Diabetes Statistics Report|Data & Statistics|Diabetes|CDC. at <www(dot)cdc(dot)gov/diabetes/data/statistics/statistics-report.html>
5. Floegel, A. et al. Identification of serum metabolites associated with risk of type 2 diabetes using a targeted metabolomic approach. Diabetes 62, 639-648 (2013).
6. Shin, S.-Y. et al. An atlas of genetic influences on human blood metabolites. Nat. Genet. 46, 543-550 (2014).
7. Long, T. et al. Whole-genome sequencing identifies common-to-rare variants associated with human blood metabolites. Nat. Genet. 49, 568-578 (2017).
8. Wikoff, W. R. et al. Metabolomics analysis reveals large effects of gut microflora on mammalian blood metabolites. Proc Natl Acad Sci USA 106, 3698-3703 (2009).
9. Fischbach, M. A. Microbiome: focus on causation and mechanism. Cell 174, 785-790 (2018).
10. Liu, R. et al. Gut microbiome and serum metabolome alterations in obesity and after weight-loss intervention. Nat. Med. 23, 859-868 (2017).
11. Fujisaka, S. et al. Diet, genetics, and the gut microbiome drive dynamic changes in plasma metabolites. Cell Rep. 22, 3072-3086 (2018).
12. Wilson, M. Microbial Inhabitants of Humans: Their ecology and role in health and disease. (Cambridge University Press, 2004). doi:10.1017/CB09780511735080
13. Topping, D. L. Short-chain fatty acids produced by intestinal bacteria. Asia Pac. J. Clin. Nutr. 5, 15-19 (1996).
14. Pedersen, H. K. et al. Human gut microbes impact host serum metabolome and insulin sensitivity. Nature 535, 376-381 (2016).
15. Patel, K. P., Luo, F. J.-G., Plummer, N. S., Hostetter, T. H. & Meyer, T. W. The production of p-cresol sulfate and indoxyl sulfate in vegetarians versus omnivores. Clin. J. Am. Soc. Nephrol. 7, 982-988 (2012).
16. Tang, W. H. W. et al. Intestinal microbial metabolism of phosphatidylcholine and cardiovascular risk. N. Engl. J. Med. 368, 1575-1584 (2013).
17. Li, X. S. et al. Gut microbiota-dependent trimethylamine N-oxide in acute coronary syndromes: a prognostic marker for incident cardiovascular events beyond traditional risk factors. Eur. Heart J. 38, 814-824 (2017).
18. Koeth, R. A. et al. Intestinal microbiota metabolism of L-carnitine, a nutrient in red meat, promotes atherosclerosis. Nat. Med. 19, 576-585 (2013).
19. Brown, J. M. & Hazen, S. L. Metaorganismal nutrient metabolism as a basis of cardiovascular disease. Curr. Opin. Lipidol. 25, 48-53 (2014).
20. Zhu, W. et al. Gut microbial metabolite TMAO enhances platelet hyperreactivity and thrombosis risk. Cell 165, 111-124 (2016).
21. Floegel, A. et al. Variation of serum metabolites related to habitual diet: a targeted metabolomic approach in EPIC-Potsdam. Eur. J. Clin. Nutr. 67, 1100-1108 (2013).
22. Thorburn, A. N., Macia, L. & Mackay, C. R. Diet, metabolites, and “western-lifestyle” inflammatory diseases. Immunity 40, 833-842 (2014).
23. Playdon, M. C. et al. Comparing metabolite profiles of habitual diet in serum and urine. Am. J. Clin. Nutr. 104, 776-789 (2016).
24. Xu, T. et al. Effects of smoking and smoking cessation on human serum metabolite profile: results from the KORA cohort study. BMC Med. 11, 60 (2013).
25. Zeevi, D. et al. Personalized nutrition by prediction of glycemic responses. Cell 163, 1079-1094 (2015).
26. Yousri, N. A. et al. Long term conservation of human metabolic phenotypes and link to heritability. Metabolomics 10, 1005-1017 (2014).
27. Ke, G. et al. LightGBM: A Highly Efficient Gradient Boosting Decision Tree. (2017).
28. Cirulli, E. T. et al. Profound Perturbation of the Metabolome in Obesity Is Associated with Health Risk. Cell Metab. 29, 488-500.e2 (2019).
29. Yousri, N. A. et al. Whole-exome sequencing identifies common and rare variant metabolic QTLs in a Middle Eastern population. Nat. Commun. 9, 333 (2018).
30. Rothschild, D. et al. Environment dominates over host genetics in shaping human gut microbiota. Nature 555, 210-215 (2018).
31. Zhernakova, A. et al. Population-based metagenomics analysis reveals markers for gut microbiome composition and diversity. Science 352, 565-569 (2016).
32. Falony, G. et al. Population-level analysis of gut microbiome variation. Science 352, 560-564 (2016).
33. Pasolli, E. et al. Extensive Unexplored Human Microbiome Diversity Revealed by Over 150,000 Genomes from Metagenomes Spanning Age, Geography, and Lifestyle. Cell 176, 649-662.e20 (2019).
34. Lundberg, S. & Lee, S.-I. A Unified Approach to Interpreting Model Predictions. arXiv (2017).
35. Lundberg, S. M., Erion, G. G. & Lee, S.-I. Consistent Individualized Feature Attribution for Tree Ensembles. arXiv (2018).
36. Manor, O. & Borenstein, E. Systematic characterization and analysis of the taxonomic drivers of functional shifts in the human microbiome. Cell Host Microbe 21, 254-267 (2017).
37. Ashihara, H., Monteiro, A. M., Gillies, F. M. & Crozier, A. Biosynthesis of caffeine in leaves of coffee. Plant Physiol. 111, 747-753 (1996).
38. Tsutsumi, Y. et al. Renal disposition of a furan dicarboxylic acid and other uremic toxins in the rat. J. Pharmacol. Exp. Ther. 303, 880-887 (2002).
39. Prentice, K. J. et al. CMPF, a Metabolite Formed Upon Prescription Omega-3-Acid Ethyl Ester Supplementation, Prevents and Reverses Steatosis. EBioMedicine 27, 200-213 (2018).
40. Hung, S.-C., Kuo, K.-L., Wu, C.-C. & Tang, D.-C. Indoxyl sulfate: A novel cardiovascular risk factor in chronic kidney disease. J. Am. Heart Assoc. 6, (2017).
41. Barrios, C. et al. Gut-Microbiota-Metabolite Axis in Early Renal Function Decline. PLoS ONE 10, e0134311 (2015).
42. Poesen, R. et al. Microbiota-Derived Phenylacetylglutamine Associates with Overall Mortality and Cardiovascular Disease in Patients with CKD. J. Am. Soc. Nephrol. 27, 3479-3487 (2016).
43. Evenepoel, P., Meijers, B. K. I., Bammens, B. R. M. & Verbeke, K. Uremic toxins originating from colonic microbial metabolism. Kidney Int. Suppl. S12-9 (2009). doi:10.1038/ki.2009.402
44. Dodd, D. et al. A gut bacterial pathway metabolizes aromatic amino acids into nine circulating metabolites. Nature 551, 648-652 (2017).
45. Atkinson, W., Downer, P., Lever, M., Chambers, S. T. & George, P. M. Effects of orange juice and proline betaine on glycine betaine and homocysteine in healthy male subjects. Eur. J. Nutr. 46, 446-452 (2007).
46. Smyth, J. M. et al. Individual differences in the diurnal cycle of cortisol. Psychoneuroendocrinology 22, 89-105 (1997).
47. Hyttel, J. Pharmacological characterization of selective serotonin reuptake inhibitors (SSRIs). Int. Clin. Psychopharmacol. 9, 19-26 (1994).
48. Korem, T. et al. Bread Affects Clinical Parameters and Induces Gut Microbiome-Associated Personal Glycemic Responses. Cell Metab. 25, 1243-1253.e5 (2017).
49. Olthof, M. R., van Vliet, T., Boelsma, E. & Verhoef, P. Low dose betaine supplementation leads to immediate and long term lowering of plasma homocysteine in healthy men and women. J. Nutr. 133, 4135-4138 (2003).
50. Craig, S. A. S. Betaine in human nutrition. Am. J. Clin. Nutr. 80, 539-549 (2004).
51. Fardet, A. et al. Whole-grain and refined wheat flours show distinct metabolic profiles in rats as assessed by a 1H NMR-based metabonomic approach. J. Nutr. 137, 923-929 (2007).
52. Chalmers, T. C. et al. A method for assessing the quality of a randomized control trial. Control. Clin. Trials 2, 31-49 (1981).
53. Yang, J. et al. Common SNPs explain a large proportion of the heritability for human height. Nat. Genet. 42, 565-569 (2010).
54. Sudlow, C. et al. UK biobank: an open access resource for identifying the causes of a wide range of complex diseases of middle and old age. PLoS Med. 12, e1001779 (2015).
55. Segata, N. et al. Metagenomic microbial community profiling using unique clade-specific marker genes. Nat. Methods 9, 811-814 (2012).
56. Li, J. et al. An integrated catalog of reference genes in the human gut microbiome. Nat. Biotechnol. 32, 834-841 (2014).
57. Zeevi, D. et al. Structural variation in the gut microbiome associates with host health. Nature (2019).
58. Bridgewater B R, E. A. High Resolution Mass Spectrometry Improves Data Quantity and Quality as Compared to Unit Mass Resolution Mass Spectrometry in High-Throughput Profiling Metabolomics. Metabolomics 04, (2014).
59. Zierer, J. et al. The fecal metabolome as a functional readout of the gut microbiome. Nat. Genet. 50, 790-795 (2018).
60. Marco-Sola, S., Sammeth, M., Guigó, R. & Ribeca, P. The GEM mapper: fast, accurate and versatile alignment by filtration. Nat. Methods 9, 1185-1188 (2012).
61. Langmead, B. & Salzberg, S. L. Fast gapped-read alignment with Bowtie 2. Nat. Methods 9, 357-359 (2012).
62. Korem, T. et al. Growth dynamics of gut microbiota in health and disease inferred from single metagenomic samples. Science 349, 1101-1106 (2015).
63. Efron, B. & Tibshirani, R. J. An Introduction to the Bootstrap. (Chapman and Hall/CRC, 1994). doi:10.1007/978-1-4899-4541-9
64. Fisher, R. A. Frequency Distribution of the Values of the Correlation Coefficient in Samples from an Indefinitely Large Population. Biometrika 10, 507 (1915).
65. Wald, A. Sequential tests of statistical hypotheses. Ann. Math. Statist. 16, 117-186 (1945).
66. GitHub—slundberg/shap: A unified approach to explain the output of any machine learning model. at <github(dot)com/slundberg/shap>
67. Shannon, P. et al. Cytoscape: a software environment for integrated models of biomolecular interaction networks. Genome Res. 13, 2498-2504 (2003).
68. Yang, J., Lee, S. H., Goddard, M. E. & Visscher, P. M. GCTA: a tool for genome-wide complex trait analysis. Am. J. Hum. Genet. 88, 76-82 (2011).
69. Schweiger, R. et al. RL-SKAT: An Exact and Efficient Score Test for Heritability and Set Tests. Genetics 207, 1275-1283 (2017).

LENGTHY TABLES
The patent application contains a lengthy table section. A copy of the table is available in electronic form from the USPTO web site (https://seqdata.uspto.gov/?pageRequest=docDetail&DocID=US20220102000A1). An electronic copy of the table will also be available from the USPTO upon request and payment of the fee set forth in 37 CFR 1.19(b)(3).

1. A method of predicting the quantity of a metabolite in the blood of a subject, the method comprising:
accessing a computer readable medium storing a library of trained machine learning procedures, each being associated with a different metabolite;
searching said library for a trained machine learning procedure associated with the metabolite;
feeding said selected procedure with amount of a plurality of microbes of a microbiome of the subject; and
receiving from said selected procedure an output indicative of the quantity of the metabolite in the blood.

2. The method of claim 1, further comprising measuring the amount of microbes of said microbiome of the subject prior to said analyzing.

3. The method according to claim 1, wherein said microbiome is a fecal microbiome.

4. The method according to claim 1, wherein said plurality of microbes comprises more than 20 microbes.

5. The method according to claim 1, wherein said metabolite is set forth in Table 2.

6. The method according to claim 1, wherein said metabolite is other than glucose and other than cholesterol.

7. (canceled)

8. The method according to claim 1, wherein at least some of said trained machine learning procedures in said library comprises a set of decision trees.

9. (canceled)

10. The method according to claim 1, wherein said selected machine learning procedure comprises a set of decision trees, each decision tree comprises a plurality of nodes associated with a respective plurality of decision rules, each decision rule relating to at least one microbe of said microbiome, and wherein a number of decision rules relating to microbes listed in Table 1 is larger than a number of decision rules relating to other microbes of said microbiome.

11. A method of predicting the quantity of a metabolite set forth in Table 1, the method comprising:
accessing a computer readable medium storing a trained machine learning procedure associated with the metabolite;
feeding said trained procedure with an amount of N of the corresponding microbes set forth in Table 1, said N being at most 50; and
receiving from said procedure an output indicative of the quantity of the metabolite in the blood, thereby predicting the quantity of the metabolite in the blood.

12. The method of claim 11, further comprising measuring the amount of microbes of said fecal microbiome of the subject prior to said analyzing.

13. (canceled)

14. A method of predicting the quantity of a metabolite in the blood of a subject that consumes a diet of a plurality of food types, the method comprising:
accessing a computer readable medium storing a library of trained machine learning procedures, each being associated with a different metabolite;
searching said library for a trained machine learning procedure associated with the metabolite;
feeding said selected procedure with a frequency of consumption of at least 5 of said food types over at least one month and/or a daily mean consumption of at least 5 of said food types; and
receiving from said selected procedure an output indicative of the quantity of the metabolite in the blood.

15. The method of claim 14, wherein said metabolite is set forth in Table 4.

16-17. (canceled)

18. The method according to claim 14, wherein at least some of said trained machine learning procedures in said library comprises a set of decision trees.

19. (canceled)

20. The method according to claim 14, wherein said selected machine learning procedure comprises a set of decision trees, each decision tree comprises a plurality of nodes associated with a respective plurality of decision rules, each decision rule relating to at least one food type, and wherein a number of decision rules relating to food types listed in Table 3 is larger than a number of decision rules relating to other food types.

21-23. (canceled)

24. The method according to claim 1, further comprising corroborating the quantity of the metabolite by measuring the amount of said metabolite in a blood sample of the subject.

25. A method of diagnosing a disease of a subject comprising predicting the quantity of at least one metabolite which is indicative of the disease, wherein said predicting is carried out according to claim 1, thereby diagnosing the disease.

26. The method of claim 25, wherein the disease is selected from the group consisting of a metabolic disease, a cardiovascular disease and kidney disease.

27-31. (canceled)

32. A method of providing dietary advice to a subject, the method comprising predicting the quantity of a metabolite in the blood by carrying out the method according to claim 14, wherein when said metabolite is above or below the recommended quantity of said metabolite, recommending consumption of at least one food type that alters the quantity of said metabolite.

33. The method of claim 32, wherein said metabolite is set forth in Table 4.

34. The method of claim 33, wherein said food type is the corresponding food type set forth in Table 4.

35-36. (canceled)
